(19) United States
(12) Patent Application Publication
(10) Pub. No.: US 2023/0357233 A1
(43) Pub. Date: Nov. 9, 2023

(54) HETEROARYL COMPOUNDS, PREPARATION METHODS AND USES THEREOF

(71) Applicants: INVENTISBIO CO., LTD., Shanghai (CN); INVENTISBIO LLC, Florham Park, NJ (US)

(72) Inventors: Xing DAI, Shanghai (CN); Hong YANG, Shanghai (CN); Xianhai HUANG, Florham Park, NJ (US); Haotao NIU, Shanghai (CN); Zixing HAN, Shanghai (CN); Zhenwu WANG, Shanghai (CN); Qiang ZHANG, Shanghai (CN); Yanqin LIU, Shanghai (CN); Yueheng JIANG, Shanghai (CN); Liangshan TAO, Shanghai (CN); Jifang WENG, Shanghai (CN); Zhe SHI, Shanghai (CN); Yaolin WANG, Shanghai (CN)

(21) Appl. No.: 18/042,872

(22) PCT Filed: Aug. 26, 2021
(86) PCT No.: PCT/CN2021/114676

§ 371 Date: Feb. 24, 2023

(30) Foreign Application Priority Data
Aug. 26, 2020 (WO) PCT/CN2020/111302
Feb. 7, 2021 (WO) PCT/CN2021/075781

(51) Int. Cl. C07D 471/04; A61K 45/06; C07D 519/00

(52) U.S. Cl. CPC C07D 471/04; A61K 45/06; C07D 519/00

(57) ABSTRACT

Provided herein are novel compounds, for example, compounds having a Formula (I), Formula (A), Formula (II), Formula (III), Formula (IV), or Formula (V), or a pharmaceutically acceptable salt thereof. Also provided herein are methods of preparing the compounds and methods of using the compounds, for example, in inhibiting KRAS^{G12D} in a cancer cell, and/or in treating various cancer such as pancreatic cancer, colorectal cancer, lung cancer (e.g., non-small cell lung cancer) or endometrial cancer.

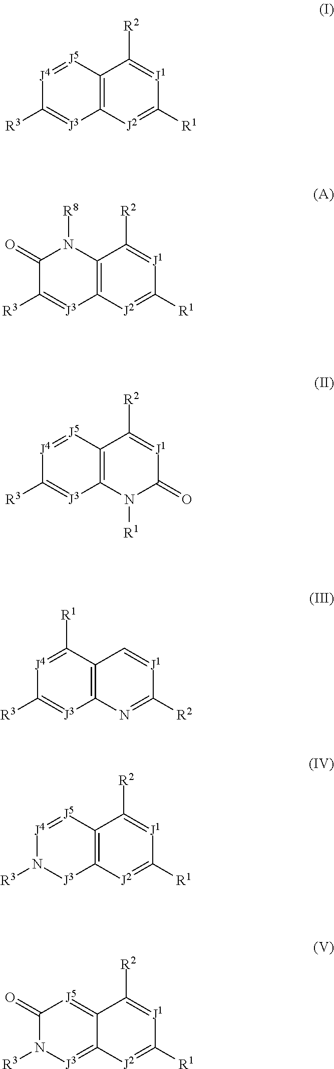

### CROSS REFERENCE TO RELATED APPLICATIONS

This application claims priority to International Application Nos. PCT/CN2020/111302, filed Aug. 26, 2020, and PCT/CN2021/075781, filed Feb. 7, 2021, the content of each of which is incorporated herein by reference in its entirety.

### BACKGROUND

### Field of the Disclosure

In various embodiments, the present disclosure generally relates to novel heteroaryl compounds, compositions comprising the same, methods of preparing and methods of using the same, e.g., for inhibiting RAS and/or for treating a number of diseases or disorders, such as cancers.

### Background

RAS (KRAS, NRAS and HRAS) proteins regulate key cellular pathway transmitting signal received from cellular membrane receptor to downstream molecules such as Raf, MEK, ERK and PI3K, which are crucial for cell proliferation and survival. RAS cycles between the inactive GDP-bound form and active GTP-bound form. RAS is frequently mutated in cancers with KRAS accounted for ˜80% of all RAS mutations. KRAS mutation occurs in approximately 86% of pancreatic cancer, 41% of colorectal cancer, 36% of lung adenocarcinoma and 20% of endometrial carcinoma (F. McCormick, 2017, Clin Cancer Res 21: 1797-1801. Cancer Genome Atlas Network, 2017, Cancer Cell 32: 185-203). The RAS hot-spot mutations occur at codons 12, 13 and 61, with 75% of KRAS mutations occurs at codon 12 (Glycine) (D. K. Simanshu, D. V. Nissley and F. McCormick, 2017, Cell, 170: 17-33). KRAS^{G12D} (change of glycine at codon 12 to aspartic acid) is frequently mutated in pancreatic adenocarcinoma, colon adenocarcinoma and lung adenocarcinoma. However, targeting the KRAS^{G12D} mutation with small molecule is a challenge due to its shallow pocket.

There is a huge unmet medical need for therapeutic intervention of cancer patients with RAS mutations.

### BRIEF SUMMARY

In various embodiments, the present disclosure provides novel compounds, pharmaceutical compositions, methods of preparing and using the same. Typically, the compounds herein are RAS inhibitors, such as mutant KRAS (e.g., G12C, G12D, G12V, or G12A, more particularly G12D) inhibitors. The compounds and compositions herein are useful for treating various diseases or disorders, such as cancer or cancer metastasis.

In some embodiments, the present disclosure provides a compound of Formula I, Formula A, Formula II, Formula III, Formula IV, or Formula V, or a pharmaceutically acceptable salt thereof:

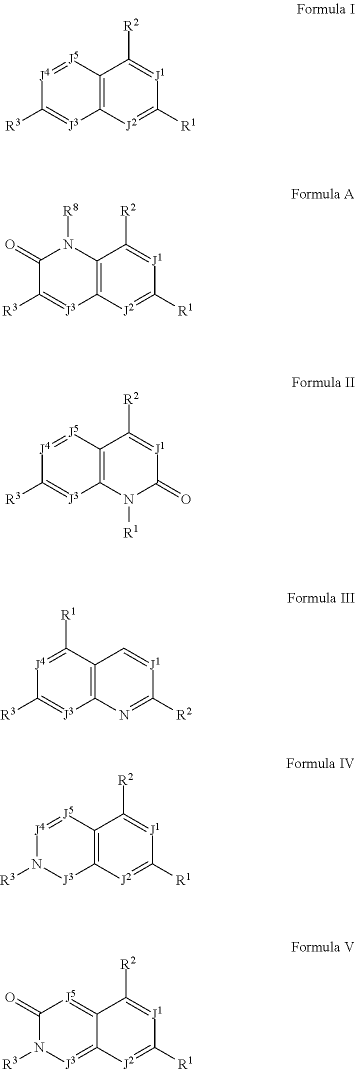

wherein R¹, R², R³, R⁸, J¹, J², J³, J⁴, and J⁵ are defined herein.

Certain embodiments of the present disclosure are directed to a pharmaceutical composition comprising one or more of the compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) and optionally a pharmaceutically acceptable excipient. The pharmaceutical composition described herein can be formulated for various routes of administration, such as oral administration, parenteral administration, or inhalation etc.

Certain embodiments are directed to a method of treating a disease or disorder associated with RAS, e.g., KRAS G12D. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein. Diseases or disorders associated with RAS, e.g., KRAS G12D, suitable to be treated with the method include those described herein.

In some embodiments, a method of treating cancer is provided. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, 1-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein. In various embodiments, the cancer can be pancreatic cancer, endometrial cancer, colorectal cancer or lung cancer (e.g., non-small cell lung cancer). In some embodiments, the cancer is a hematological cancer (e.g., described herein). In some embodiments, the cancer can be appendix cancer, cholangiocarcinoma, bladder urothelial cancer, ovarian cancer, gastric cancer, breast cancer, or bile duct cancer.

In some embodiments, a method of treating cancer metastasis or tumor metastasis is provided. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally.

The compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, and/or immunotherapy.

It is to be understood that both the foregoing summary and the following detailed description are exemplary and explanatory only, and are not restrictive of the invention herein.

### DETAILED DESCRIPTION

International application No. PCT/CN2020/099104, filed Jun. 30, 2020 by Inventisbio Shanghai Ltd., the content of which is herein incorporated by reference in its entirety, describes certain quinazoline compounds as useful agents for inhibiting RAS (e.g., KRAS^{G12D}) and/or for treating a number of diseases or disorders such as cancer. It has now been discovered that a quinazoline core is not required for RAS inhibition. In various embodiments, provided herein are novel heteroaryl compounds, pharmaceutical compositions, methods of preparation and methods of use.

### Compounds

Some embodiments of the present disclosure are directed to novel compounds. The compounds herein typically can be an inhibitor of a KRAS protein, particularly, a KRAS G12D mutant protein, and useful for treating various diseases or disorders, such as those described herein, e.g., cancer.

In some embodiments, the present disclosure provides a compound of Formula I, or a pharmaceutically acceptable salt thereof:

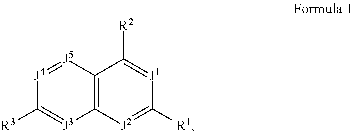

wherein:
J¹ is CR⁹ or N;
J² is CR¹⁰ or N;
J³ is CR¹¹ or N;
J⁴ is CR¹² or N;
J⁵ is CR^{12A} or N;
provided that at least one of J¹ and J² is N, and when both J¹ and J² are N, then at least one of J³, J⁴ and J⁵ is N;
or J⁴ and J⁵ are joined to form an optionally substituted phenyl or optionally substituted 5, or 6-membered heteroaryl, provided that in such cases, the bond between J⁴ and J⁵ can be a single bond, for example, when J⁴ and J⁵ are joined to form a triazole ring;
R¹ is hydrogen, -(Li)ₘ₁-OR²⁰, halogen, -(L¹)ₘ₁-NR³⁰R³¹, —C(O)—NR³⁰R³¹, optionally substituted alkyl, or an optionally substituted heterocyclic or heteroaryl ring;
R² is a ring or ring-chain structure, e.g., those having a basic functional group with a pKa of the conjugated acid of about 5 or higher, or an acylated derivative thereof (i.e., the basic functional group, such as a basic NH, is bonded with an acyl group);
R³ is an optionally substituted aryl or an optionally substituted heteroaryl,
R⁹ and R¹⁰ are each independently hydrogen, halogen, cyano, optionally substituted C₁₋₄ alkyl (e.g., methyl, ethyl, CF₃, etc.), optionally substituted C₂₋₄ alkenyl, optionally substituted C₂₋₄ alkynyl, optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹, R¹² and R^{12A} are each independently hydrogen, F, Cl, Br, I, CN, —OH, —C(O)NH₂, —NH₂, —NH(C₁₋₆ alkyl), —N(C₁₋₆ alkyl)(C₁₋₆ alkyl), —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), optionally substituted C₂₋₄ alkenyl, optionally substituted C₂₋₄ alkynyl, optionally substituted C₃₋₆ cycloalkyl (e.g., cyclopropyl or cyclobutyl), optionally substituted C₁₋₄ alkoxy (e.g., methoxy, ethoxy, —O—CH₂-cyclopropyl), optionally substituted C₃₋₆ cycloalkoxy (e.g., cyclopropoxy, or cyclobutoxy), optionally substituted 4-7 membered heterocyclic, or optionally substituted 4-7 membered heterocycloalkoxy;
or R¹² and R^{12A} are joined to form a 5-7 membered ring structure; and
   wherein:
   m1 is 0 or 1, and when m1 is 1, L¹ is an optionally substituted alkylene, an optionally substituted carbocyclylene, an optionally substituted heterocyclylene;
   R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
   R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring, or one of R³⁰ and R³¹ together with a suitable atom of L¹ and any intervening atoms form an optionally substituted heterocyclic or heteroaryl ring.

The compound of Formula I (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as a mixture of atropisomers in any ratio, including about 1:1. In some embodiments, when applicable, the compound of Formula I (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer.

In some embodiments, J¹ in Formula I is CR⁹. Although various groups are suited for R⁹, in Formula I, R⁹ is typically H. In some embodiments, J¹ in Formula I is N. In some embodiments, J² in Formula I is CR¹⁰, such as CH. In some embodiments, J² in Formula I is N. In some embodiments, J³ in Formula I is CR¹¹, such as CH or C—F. In some embodiments, J³ in Formula I is N. In some embodiments, J⁴ in Formula I is CR¹², such as CH or C—CN. In some embodiments, J⁴ in Formula I is N. In some embodiments, J⁵ in Formula I is CR^{12A} such as CH or C-Me. In some embodiments, J⁵ in Formula I is N. In some embodiments, J⁴ and J⁵ are joined to form an optionally substituted 5, or 6-membered heteroaryl, provided that in such cases, the bond between J⁴ and J⁵ can be a single bond. For example, in some embodiments, J⁴ and J⁵ are joined to form a triazole ring, see e.g., Formula I-24 below.

Combinations of J¹, J², J³, J⁴, and J⁵ are not particularly limited. For example, in some embodiments, the compound of Formula I can have one of the following subformulae:

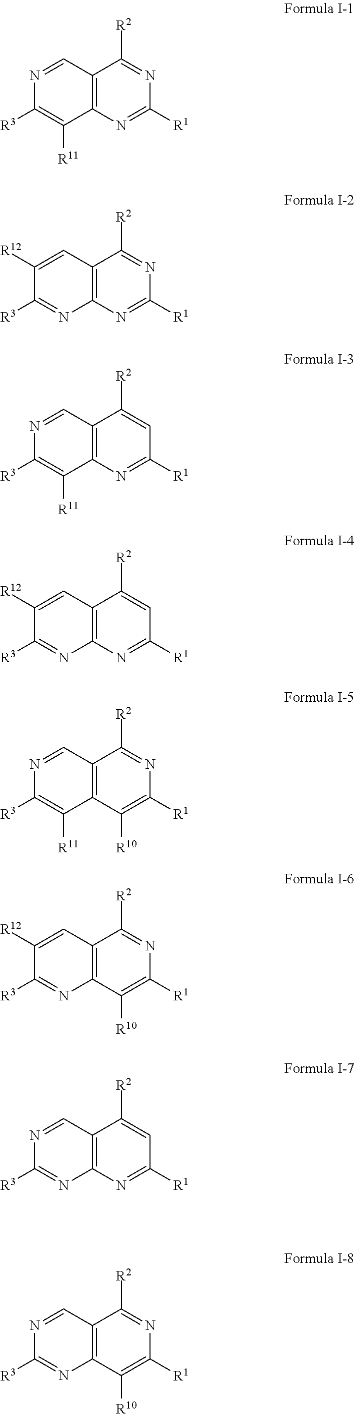

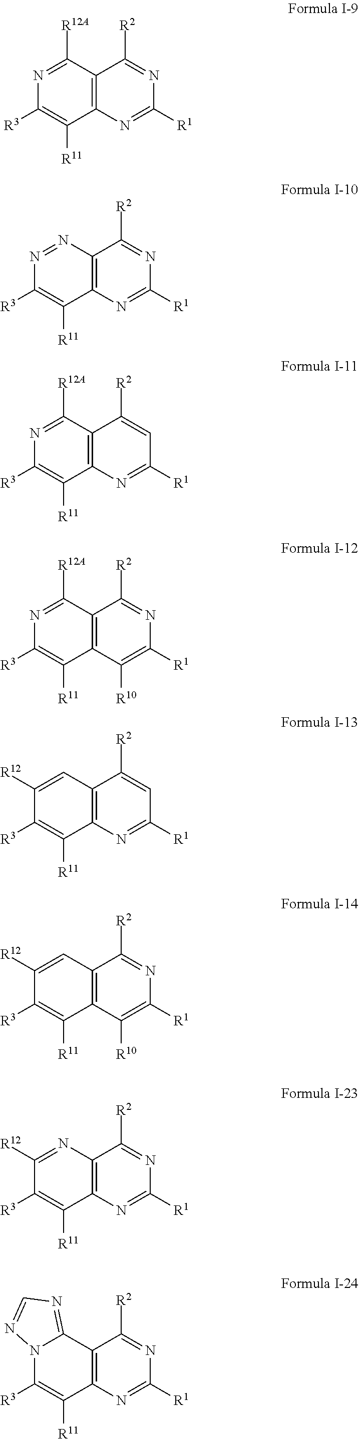

wherein R¹, R², R³, R¹⁰, R¹¹, R¹² and R^{12A} include any of those defined herein in any combinations.

In some embodiments, when present, R¹⁰ in Formula I (e.g., Formula I-5, I-6, I-8, I-12, or I-14) is hydrogen, halogen (e.g., Cl), C₁₋₄ alkyl optionally substituted with 1-3 F, e.g., methyl, ethyl, CF₃, etc., cyclopropyl, cyclobutyl, 5- or 6-membered heteroaryl having 1-4 heteroatoms independently selected from N, O, and S, such as pyrrazolyl, oxazolyl, pyridyl, pyrimidinyl, pyridazinyl, pyrazinyl, etc., wherein the heteroaryl is optionally substituted with 1-3 substituents independently selected from halogen, CN, C₁₋₄ alkyl optionally substituted with 1-3 F, e.g., methyl, ethyl, CF₃, etc., C₃₋₆ cycloalkyl (e.g., cyclopropyl, cyclobutyl) optionally substituted with one or more substituents independently selected from methyl, F, OH, and methoxy, and C₁₋₄ alkoxy optionally substituted with 1-3 F, e.g., methoxy, ethoxy, —OCF₃, etc.

In some embodiments, when present, R¹⁰ in Formula I (e.g., Formula I-5, I-6, I-8, I-12 or I-14) is hydrogen, F, Cl, methyl, ethyl, isopropyl, CF₃, cyclopropyl, or cyclobutyl.

In some embodiments, when present, R¹⁰ in Formula I (e.g., Formula I-5, I-6, I-8, I-12 or I-14) is

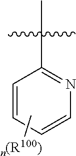

wherein R¹⁰⁰ at each occurrence is independently halogen, CN, C₁₋₄ alkyl optionally substituted with 1-3 F, e.g., methyl, ethyl, CF₃, etc., C₃₋₆ cycloalkyl (e.g., cyclopropyl, cyclobutyl) optionally substituted with one or more substituents independently selected from methyl, F, OH, and methoxy, and C₁₋₄ alkoxy optionally substituted with 1-3 F, e.g., methoxy, ethoxy, —OCF₃, etc.; and n is 0, 1, 2, or 3, preferably, n is 0, 1, or 2.

Suitable R¹⁰ for Formula I (e.g., Formula I-5, I-6, I-8, I-12 or I-14) also include those exemplified herein in the specific examples.

In some embodiments, when present, R¹¹ in Formula I (e.g., Formula I-1, I-3, I-5, I-9, I-10, I-11, I-12, I-13, I-14, I-23 or I-24) is F, Cl, —CN, —OH, methoxy, ethoxy, —O—CH₂-cyclopropyl, —C(O)NHMe, CF₃, methyl, ethyl, isopropyl, or cyclopropyl. For example, in some embodiments, R¹¹ in Formula I (e.g., Formula I-1, I-3, I-5, I-9, I-10, I-11, I-12, I-13, I-14, I-23 or I-24) is F. In some embodiments, R¹¹ in Formula I (e.g., Formula I-1, I-3, I-5, I-9, I-10, I-11, I-12, I-13, I-14, I-23 or I-24) is Cl. In some embodiments, R¹¹ in Formula I (e.g., Formula I-1, I-3, I-5, I-9, I-10, I-11, I-12, I-13, I-14, I-23 or I-24) is methyl. In some embodiments, R¹¹ in Formula I (e.g., Formula I-1, I-3, I-5, I-9, I-10, I-11, I-12, I-13, I-14, I-23 or I-24) is cyclopropyl. In some embodiments, R¹¹ in Formula I (e.g., Formula I-1, I-3, I-5, I-9, I-10, I-11, I-12, I-13, I-14, I-23 or I-24) is hydrogen. Suitable R¹¹ for Formula I (e.g., Formula I-1, I-3, I-5, I-9, I-10, I-11, I-12, I-13, I-14, I-23 or I-24) also include those exemplified herein in the specific examples.

In some embodiments, when present, R¹² in Formula I (e.g., Formula I-2, I-4, I-6, I-13, I-14, or I-23) is F, Cl, —CN, —OH, methoxy, ethoxy, —O—CH₂-cyclopropyl, —C(O)NHMe, CF₃, methyl, ethyl, isopropyl, or cyclopropyl. In some embodiments, R¹² in Formula I (e.g., Formula I-2, I-4, I-6, I-13, I-14, or I-23) is F. In some embodiments, R¹² in Formula I (e.g., Formula I-2, I-4, I-6, I-13, I-14, or I-23) is Cl. Suitable R¹² for Formula I (e.g., Formula I-1, I-3, I-5, I-13, I-14, or I-23) also include those exemplified herein in the specific examples.

In some embodiments, when present, R^{12A} in Formula I (e.g., Formula I-9, I-11, or I-12) is hydrogen. In some embodiments, when present, R^{12A} in Formula I (e.g., Formula I-9, I-11, or I-12) is halogen, such as Cl. In some embodiments, when present, R^{12A} in Formula I (e.g., Formula I-9, I-11, or I-12) is optionally substituted C₁₋₄ alkyl (e.g., methyl, ethyl, CHF₂, CF₃, etc.), when substituted, the C₁₋₄ alkyl is typically substituted with 1-3 substituents independently selected from F, OH, C₁₋₄ alkoxy optionally substituted with 1-3 F, cyclopropyl, cyclobutyl, CONH(C₁₋₄ alkyl), CONH₂, CON(C₁₋₄ alkyl)(C₁₋₄ alkyl), and 4-7 membered heterocyclic having 1 or 2 ring heteroatoms independently O, N, or S. In some embodiments, when present, R^{12A} in Formula I (e.g., Formula I-9, I-11, or I-12) is optionally substituted C₃₋₆ cycloalkyl (e.g., cyclopropyl or cyclobutyl), when substituted, the C₃₋₆ cycloalkyl is typically substituted with 1-3 substituents independently selected from F, OH, methyl, hydroxymethyl, CHF₂, CH₂F, CF₃, and C₁₋₄ alkoxy optionally substituted with 1-3 F. In some embodiments, when present, R^{12A} in Formula I (e.g., Formula I-9, I-11, or I-12) is optionally substituted C₁₋₄ alkoxy (e.g., methoxy, ethoxy, difluoromethoxy, trifluoromethoxy, difluoroethoxy, trifluorethoxy, —O—CH₂—CH₂-cyclopropyl, —O—CH₂-cyclopropyl), when substituted, the C₁₋₄ alkoxy is typically substituted with 1-3 substituents independently selected from F, OH, C₁₋₄ alkyl optionally substituted with 1-3 F, C₁₋₄ alkoxy optionally substituted with 1-3 F, cyclopropyl, cyclobutyl, CONH(C₁₋₄ alkyl), CONH₂, CON(C₁₋₄ alkyl)(C₁₋₄ alkyl), and 4-7 membered heterocyclic having 1 or 2 ring heteroatoms independently O, N, or S. In some embodiments, when present, R^{12A} in Formula I (e.g., Formula I-9, I-11, or I-12) is optionally substituted C₃₋₆ cycloalkoxy (e.g., cyclopropoxy, or cyclobutoxy), when substituted, the C₃₋₆ cycloalkoxy is typically substituted with 1-3 substituents independently selected from F, OH, methyl, hydroxymethyl, CHF₂, CH₂F, CF₃, and C₁₋₄ alkoxy optionally substituted with 1-3 F. In some embodiments, when present, R^{12A} in Formula I (e.g., Formula I-9, I-11, or I-12) is optionally substituted 4-7 membered heterocyclic, such as a monocyclic 4-7 membered heterocyclic ring described herein, when substituted, the 4-7 membered heterocyclic ring is typically substituted with 1-3 substituents independently selected from F, oxo, OH, methyl, hydroxymethyl, CHF₂, CH₂F, CF₃, and C₁₋₄ alkoxy optionally substituted with 1-3 F. In some embodiments, when present, R^{12A} in Formula I (e.g., Formula I-9, I-11, or I-12) is optionally substituted 4-7 membered heterocycloalkoxy, when substituted, the 4-7 membered heterocycloalkoxy is typically substituted with 1-3 substituents independently selected from F, oxo, OH, methyl, hydroxymethyl, CHF₂, CH₂F, CF₃, and C₁₋₄ alkoxy optionally substituted with 1-3 F. As used herein, a heterocycloalkoxy refers to —O—R, wherein R is a heterocyclic ring defined herein. In some embodiments, when present, R^{12A} in Formula I (e.g., Formula I-9, I-11, or I-12) can also be —NH₂, —NH(C₁₋₆ alkyl), or —N(C₁₋₆ alkyl)(C₁₋₆ alkyl).

In some embodiments, when applicable, R¹² and R^{12A} in Formula I can also be joined to form a 5-7 membered ring structure.

In some embodiments, when present, R^{12A} in Formula I (e.g., Formula I-9, I-11, or I-12) is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, R^{12A} in Formula I (e.g., Formula I-9, I-11, or I-12) is H or C₁₋₄ alkyl optionally substituted with F, such as methyl. In some embodiments, R^{12A} in Formula I (e.g., Formula I-9, I-11, or I-12) is Cl or methoxy. In some embodiments, R^{12A} in Formula I (e.g., Formula I-9, I-11, or I-12) is ethyl or difluoromethyl. In some embodiments, R^{12A} in Formula I (e.g., Formula I-9, I-11, or I-12) is OH In some embodiments, R^{12A} in Formula I (e.g., Formula I-9, I-11, or I-12) is halogen, —OH, C₁₋₄ alkyl optionally substituted with 1-3 F, C₁₋₄ alkoxy optionally substituted with 1-3 F, or cyclopropyl substituted C₁₋₄ alkoxy, which is optionally substituted 1-3 F. In some embodiments, R^{12A} in Formula I (e.g., Formula I-9, I-11, or I-12) is Cl, —OH, methoxy, difluoromethoxy, ethoxy, isopropoxy, —O—CH₂-cyclopropyl, —O—CH₂—CH₂-cyclopropyl, —C(O)NHMe, —O—CH₂—C(O)NHMe, —O—CH₂—CF₃, —O—CH₂—CHF₂, methyl, CHF₂, CF₃, ethyl, isopropyl, or cyclopropyl. Suitable R^{12A} for Formula I (e.g., Formula I-9, I-11, or I-12) also include those exemplified herein in the specific examples.

In some embodiments, the compound of Formula I can have one of the following subformulae:

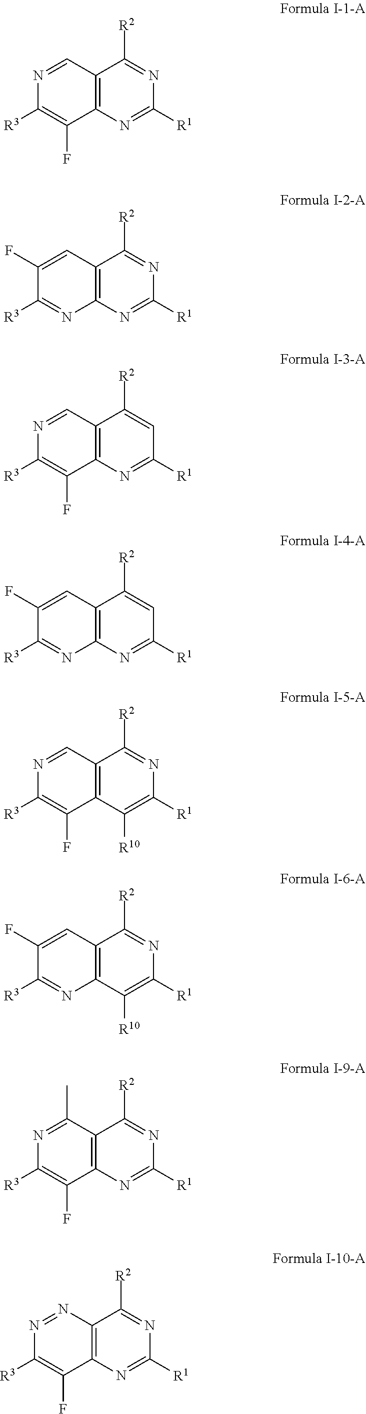

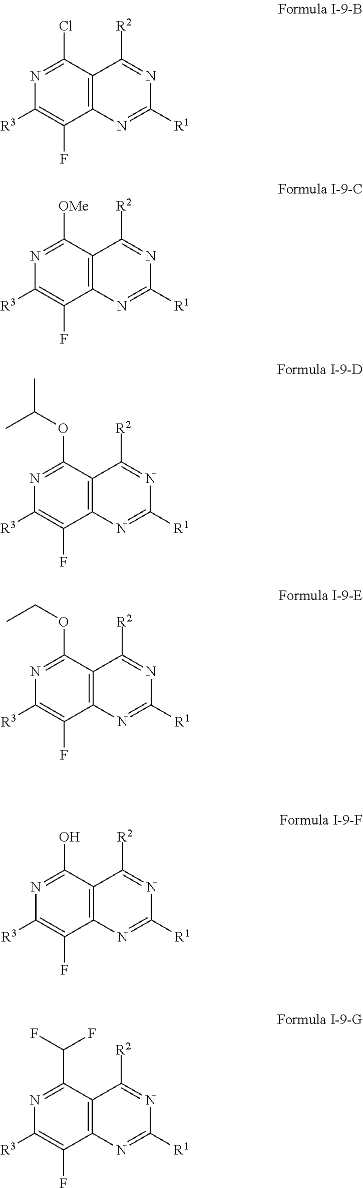

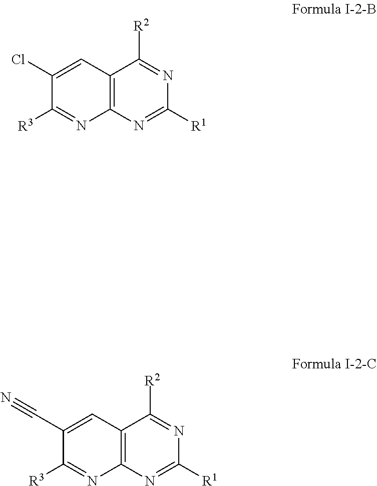

wherein R¹, R², R³, and R¹⁰, include any of those defined herein in any combinations.

Various groups are suitable as R¹ in Formula I. In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be hydrogen. In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be a halogen, such as F or Cl. In some embodiments, suitable R¹ for Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) also include any of those exemplified herein in the specific examples.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be optionally substituted alkyl, such as an optionally substituted C₁₋₄ alkyl. For example, in some embodiments, R¹ in Formula I can be C₁₋₄ alkyl optionally substituted with 1-3 F. In some embodiments, R¹ in Formula I can be methyl, CHF₂, or CF₃.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be —C(O)—NR³⁰R³¹, such as CONH(C₁₋₄ alkyl), wherein the C₁₋₄ alkyl is optionally substituted. For example, in some embodiments, R¹ in Formula I can be

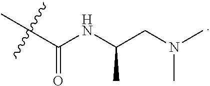 [SMILES: *C(=O)N[C@H](C)CN(C)C]

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be -(L¹)ₘ₁-OR²⁰. In some embodiments, m1 is 0, i.e., R¹ is —OR²⁰. In some embodiments, m1 is 1, and L¹ can be an optionally substituted C₁₋₄ alkylene, an optionally substituted C₃₋₆ carbocyclylene, an optionally substituted 3-7 membered heterocyclylene. For example, in some embodiments, m1 is 1, and L¹ can be a C₁₋₄ alkylene such as —CH₂—, —CH₂—CH₂—, or —CH₂—CH₂—CH₂—.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is —OR²⁰, wherein R²⁰ is a —C₁₋₆ alkylene-R¹⁰¹, wherein R¹⁰¹ is NR³²R³³ or an optionally substituted 4-10 membered heterocyclic ring, wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring; R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represents a monoalkyl or dialkyl amine; or R³² and R³³ are joined to form an optionally substituted heterocyclic or heteroaryl ring; and R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring. In some embodiments, the —C₁₋₆ alkylene-unit in R²⁰ is unsubstituted C₁₋₄ alkylene (straight chain or branched). In some embodiments, the —C₁₋₆ alkylene-unit in R²⁰ is a C₁₋₄ alkylene optionally substituted with 1, 2, or 3 substituents, preferably 1 or 2 substituents, independently selected from F, —OH, methyl, ethyl, and CF₃. In some embodiments, the —C₁₋₆ alkylene-unit in R²⁰ is a C₁₋₄ alkylene, wherein two substituents (e.g., of the same carbon) are joined to form a cyclopropyl, cyclobutyl, or a 5-6 membered heterocyclic ring such as pyrrolidine, piperidine, tetrahydrofuran, tetrahydropyran ring, which ring may be optionally substituted with substituents such as F, —OH, methyl, ethyl, and CF₃. In some embodiments, the —C₁₋₆ alkylene-unit in R²⁰ is selected from —CH₂—, —CH₂—CH₂—, —CH₂—CH₂—CH₂—,

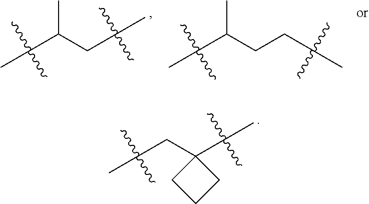

In some embodiments, the —C₁₋₆ alkylene-unit in R²⁰ is

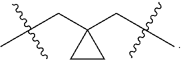 [SMILES: *CC1(C*)CC1]

As used herein, when unspecified, the divalent structures can connect to the remainder of the molecule through either direction. In some embodiments, R²⁰ is —CH₂—R¹⁰¹, —CH₂—CH₂—R¹⁰¹, —CH₂—CH₂—CH₂—R¹⁰¹,

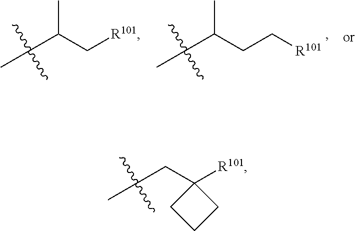

wherein R¹⁰¹ is defined herein. In some embodiments, the —C₁₋₆ alkylene-unit in R²⁰ is

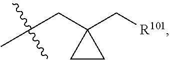

wherein R¹⁰¹ is defined herein.

R¹⁰¹ is typically NR³²R³³ or an optionally substituted 4-10 membered heterocyclic ring having 1-3 ring heteroatoms independently selected from O, S, and N. Typically, the heterocyclic ring is a saturated heterocyclic ring, which is optionally substituted.

In some embodiments, R¹⁰¹ is NR³²R³³, wherein R³² and R³³ are independently hydrogen or an optionally substituted C₁₋₄ alkyl, such as methyl, ethyl, isopropyl, etc. For example, in some embodiments, R¹⁰¹ is NH₂, NH(C₁₋₄ alkyl), or N(C₁₋₄ alkyl)(C₁₋₄ alkyl). As used herein, the two C₁₋₄ alkyl in N(C₁₋₄ alkyl)(C₁₋₄ alkyl) can be the same or different, for example, it includes N(CH₃)₂ and N(CH₃)(C₂H₅), etc. Other similar expressions should be understood similarly. In some embodiments, R¹⁰¹ is NR³²R³³, wherein one of R³² and R³³ is hydrogen or an optionally substituted C₃₋₆ cycloalkyl, and the other of R³² and R³³ is defined herein, for example, in some embodiments, the other of R³² and R³³ is hydrogen, an optionally substituted C₃₋₆ cycloalkyl, or a C₁₋₄ alkyl such as methyl. In some embodiments, R¹⁰¹ is NR³²R³³, wherein one of R³² and R³³ is hydrogen or an optionally substituted 4-8 membered heterocyclic ring such as those having 1 or 2 heteroatoms independently selected from O and N, preferably, the ring has at most one oxygen, and the other of R³² and R³³ is defined herein, for example, in some embodiments, the other of R³² and R³³ is hydrogen or a C₁₋₄ alkyl such as methyl. In some embodiments, R¹⁰¹ is NR³²R³³, wherein one of R³² and R³³ is hydrogen or a C₁₋₄ alkyl and the other of R³² and R³³ can be a C₁₋₃₀ alkyl. For example, in some embodiments, R¹⁰¹ can be NH(C₁₋₃₀ alkyl) or N(C₁₋₄ alkyl)(C₁₋₃₀ alkyl), e.g., N(CH₃)(C₁₋₃₀ alkyl).

In some embodiments, R¹⁰¹ is NR³²R³³, wherein R³² and R³³ together with the N they are both attached to are joined to form an optionally substituted 4-8 membered monocyclic heterocyclic ring having one or two ring heteroatoms, e.g., one ring nitrogen atom, two ring nitrogen atoms, one ring nitrogen atom and one ring sulfur atom, or one ring nitrogen atom and one ring oxygen atom, etc. For example, in some embodiments, R¹⁰¹ is NR³²R³³, wherein R³² and R³³ together with the N they are both attached to are joined to form a ring selected from

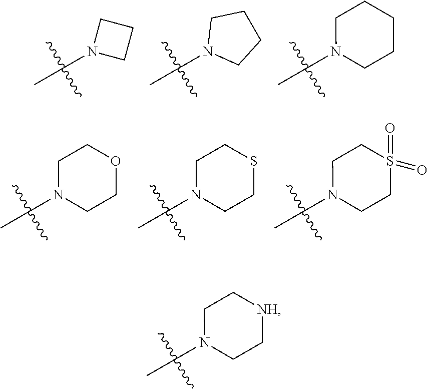

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. The substituents can be attached to any available positions in the ring, including for example an available ring nitrogen atom.

In some embodiments, R¹⁰¹ is NR³²R³³, wherein R³² and R³³ together with the N they are both attached to are joined to form a

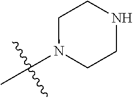 [SMILES: *N1CCNCC1]

ring, which is optionally substituted with one or more (e.g., 1 or 2) substituents independently selected from F, —OH, C₁₋₄ alkoxy optionally substituted with 1-3 fluorine, oxo, acyl, amide, ester, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S. For example, in some embodiments, the piperazine ring can have a substituent attached to one of the ring nitrogens, which can be a C₁₋₄ alkyl, an acyl, such as —C(O)(C₁₋₃₀ alkyl), an ester, such as —C(O)—O—(C₁₋₃₀ alkyl), or an amide, such as —C(O)—NH(C₁₋₃₀ alkyl) or —C(O)—N(C₁₋₄ alkyl)(C₁₋₃₀ alkyl). For example, in some embodiments, R¹⁰¹ can be

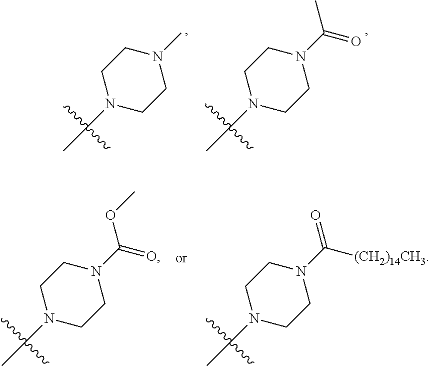

In some embodiments, R¹⁰¹ can be a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted, e.g., with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. The monocyclic or bicyclic ring can be attached to the —C₁₋₆ alkylene-moiety via any available position to form a R²⁰. For the bicyclic ring, the attaching point can be on either of the two rings, including the bridging atoms and bridgehead atoms as applicable.

For example, in some embodiments, R¹⁰¹ can be a monocyclic ring selected from the following:

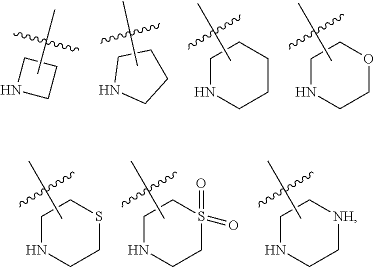

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹⁰¹ can be a bicyclic ring selected from the following:

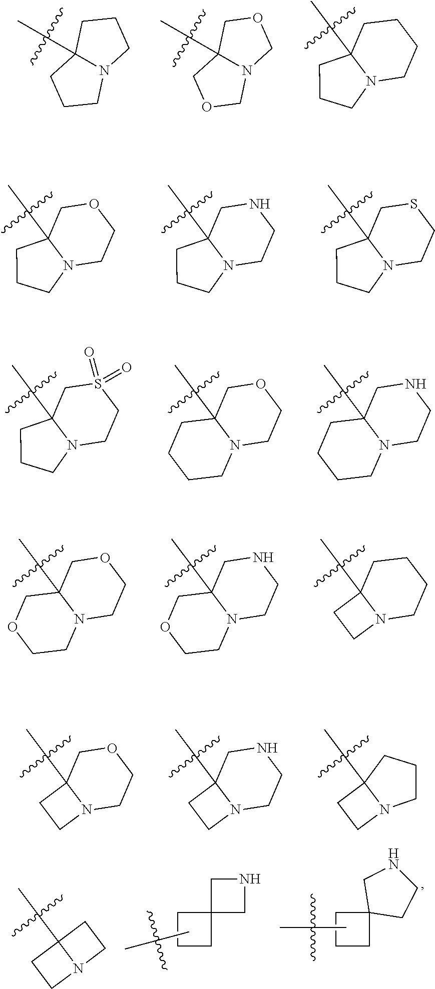

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. To be clear, the attaching point of the two spiro-bicyclic structure above can be a ring atom from either the cyclobutyl ring or the azetidine or pyrrolidine ring. In some embodiments, the attaching point is a ring atom from the cyclobutyl ring, e.g., on the carbon that's not adjacent to the spiro center. In some embodiments, R¹⁰¹ can also be a bridged bicyclic structure, such as those containing 1 or 2 ring heteroatoms independently selected from nitrogen and oxygen, such as those having 1 ring nitrogen, or those having 1 ring nitrogen and 1 ring oxygen, or those having two ring nitrogens, wherein the bridged bicyclic system can be, e.g., a [2,2,1], [2,2,2], [3,1,1], or [3,2,1] bridged bicyclic system. The bridged bicyclic structure can be optionally substituted, e.g., with one or more (e.g., 1, 2 or 3) substituents independently selected from F, methyl, ethyl, isopropyl, cyclopropyl, —N(CH₃)₂, —OH, and —OCH₃.

In some embodiments, R¹⁰¹ can be NH₂, NH(C₁₋₃₀ alkyl), N(CH₃)(C₁₋₃₀ alkyl),

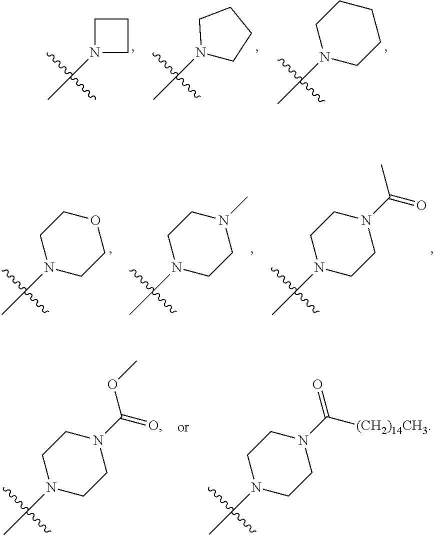

Any of the R¹⁰¹ can be combined with any of the —C₁₋₆ alkylene-moiety described herein to form a R²⁰ suitable for Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), wherein R¹ is —OR²⁰. For example, in some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be selected from:

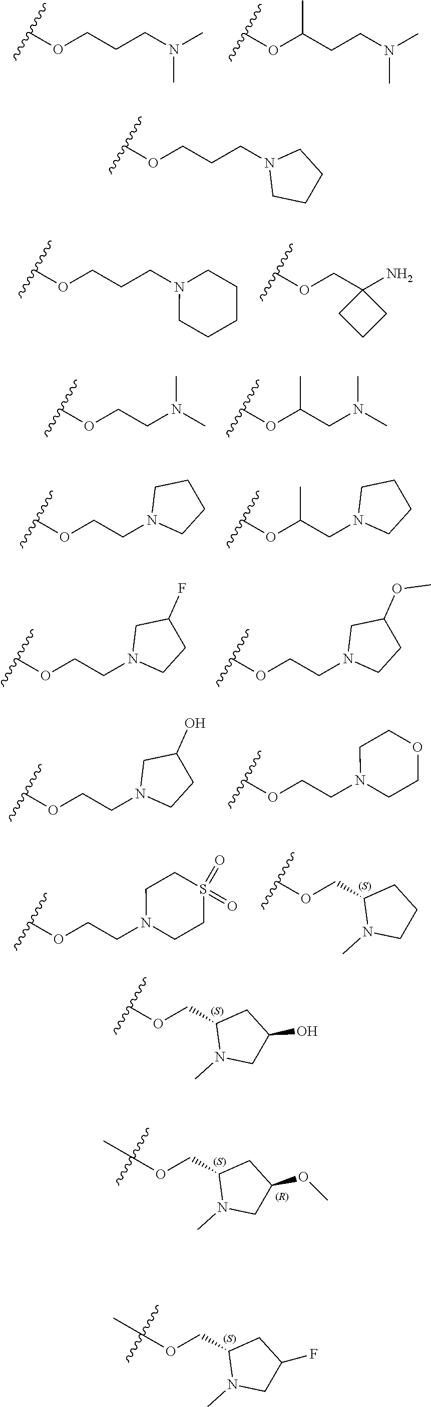

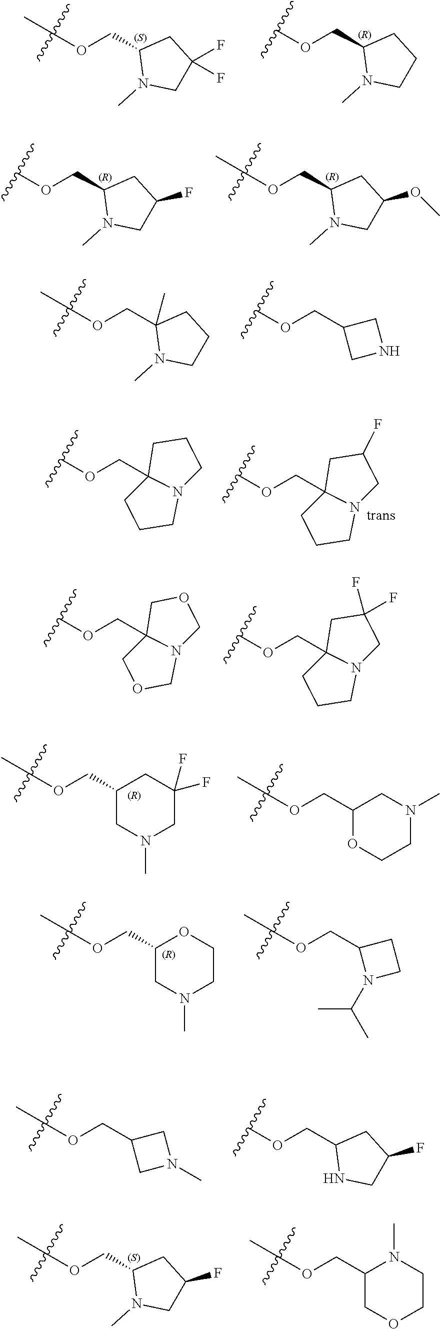

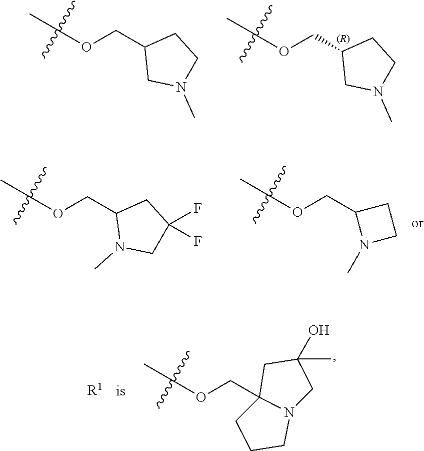

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be methoxy,

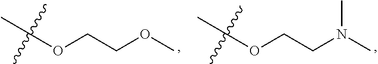

NH₂, NH(CH₃), or N (CH₃)₂.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

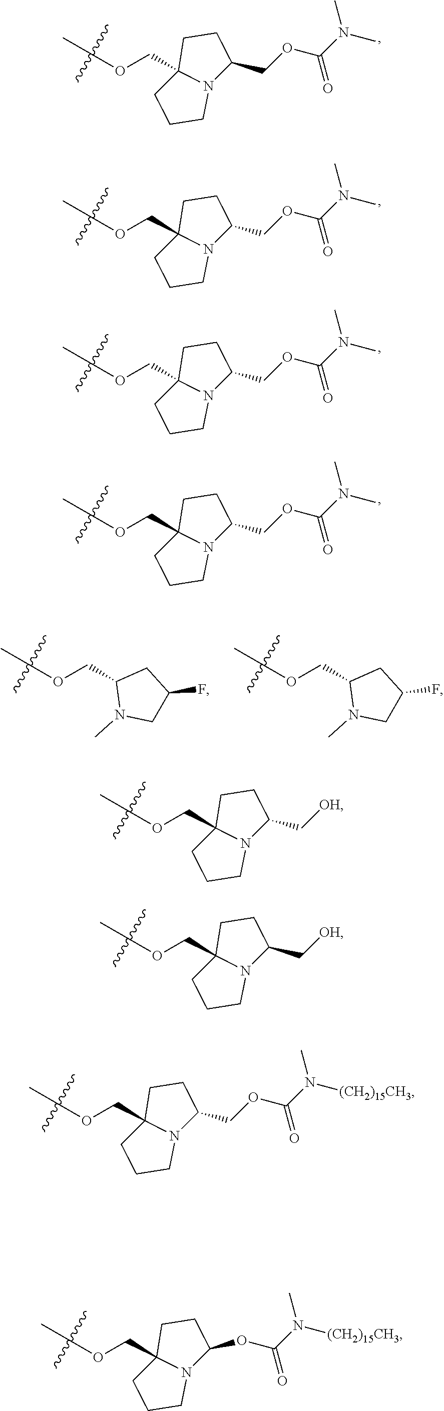

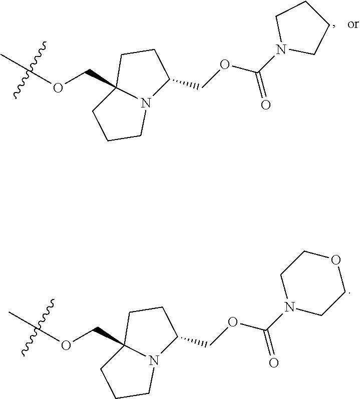 [SMILES: *OC[C@]12CCCN1[C@@H](COC(=O)N1CCCC1)CC2 ; *OC[C@]12CCCN1[C@@H](COC(=O)N1CCOCC1)CC2]

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

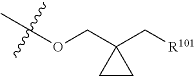

and R¹⁰¹ is NH₂, NH(C₁₋₃₀ alkyl), N(CH₃)(C₁₋₃₀ alkyl),

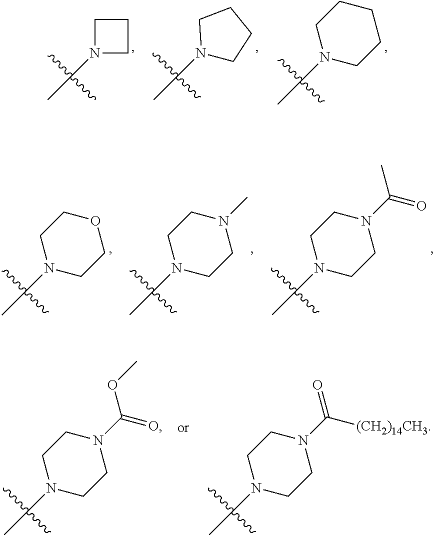

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be —OR²⁰, wherein R²⁰ is an optionally substituted C₃₋₆ carbocyclic ring or 4-10 membered heterocyclic ring. The oxygen can be connected with the carbocyclic or heterocyclic ring via any available attaching point, however, typically not through a heteroatom or a carbon atom adjacent to a heteroatom. In some embodiments, R²⁰ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted, e.g., with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some embodiments, R²⁰ is a 4-8 membered monocyclic saturated ring having one ring heteroatom, a ring nitrogen. For example, in some embodiments, R²⁰ is a monocyclic saturated ring selected from the following:

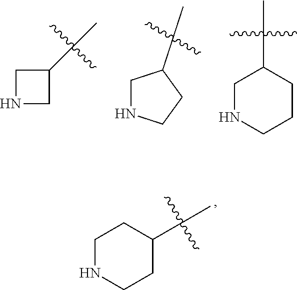 [SMILES: *C1CNC1 ; *C1CCNC1 ; *C1CCCNC1 ; *C1CCNCC1]

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, tetrahydropyranyl, —N(CH₃)₂, —OH, and —OCH₃.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be —OR²⁰, wherein R²⁰ is an optionally substituted aryl or heteroaryl ring.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be selected from the following:

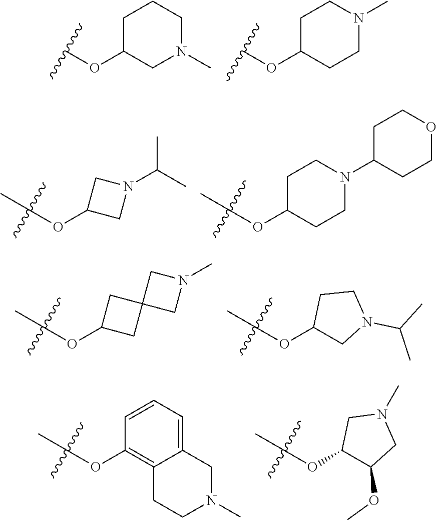

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be -(L¹)ₘ₁-NR³⁰R³¹. In some embodiments, m1 is 0, i.e., R¹ is NR³⁰R³¹. In some embodiments, m1 is 1, and L¹ can be an optionally substituted C₁₋₆ alkylene, an optionally substituted C₃₋₆ carbocyclylene, an optionally substituted 3-7 membered heterocyclylene. For example, in some embodiments, m1 is 1, and L¹ can be a C₁₋₄ alkylene such as —CH₂—, —CH₂—CH₂—, or —CH₂—CH₂—CH₂—.

For example, in some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be NR³OR³¹ or —C₁₋₆ alkylene-NR³OR³¹. In some embodiments, R³⁰ and R³¹ are independently hydrogen, an optionally substituted C₁₋₆ alkyl, or an optionally substituted heterocyclic ring; or R³⁰ and R³¹ together with the N they are both attached to are joined to form an optionally substituted heterocyclic ring having one or two ring heteroatoms, or one of R³⁰ and R³¹ together with a CH₂ unit of the C₁₋₆ alkylene and any intervening atoms form an optionally substituted heterocyclic or heteroaryl ring having one or two ring heteroatoms. In some embodiments, one of R³⁰ and R³¹ is an optionally substituted 4-8 membered monocyclic saturated heterocyclic ring such as those having 1 or 2 heteroatoms independently selected from O and N, preferably, the ring has at most one oxygen. In some embodiments, the 4-8 membered monocyclic saturated heterocyclic ring is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, the 4-8 membered monocyclic saturated heterocyclic ring has one ring heteroatom, which is a ring nitrogen atom (e.g., azetidine, pyrrolidine, piperazine, etc.). Typically, the attaching point is not the ring nitrogen atom or a carbon atom adjacent to the ring nitrogen. In some embodiments, the other of R³⁰ and R³¹ is hydrogen or an optionally substituted C₁₋₆ alkyl, such as C₁₋₄ alkyl, e.g., methyl, ethyl, or isopropyl.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be —C₁₋₆ alkylene-NR³⁰R³¹, wherein R³⁰ and R³¹ together with the N they are both attached to are joined to form a ring selected from

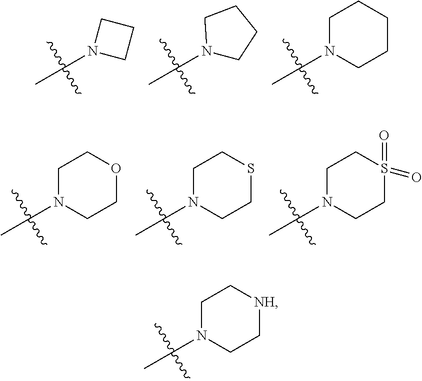

each of which is optionally substituted, for example, optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be —C₁₋₆ alkylene-NR³⁰R³¹, wherein R³⁰ together with a CH₂ unit of the C₁₋₆ alkylene and any intervening atoms form a ring selected from (R³¹ is shown):

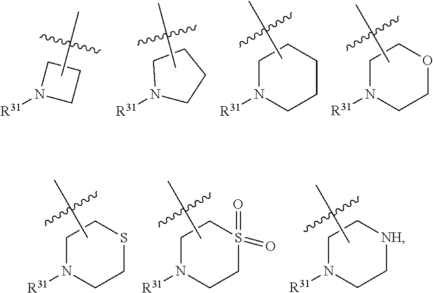

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, R³¹ is —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH, —(CH₂)ₓ—NH(C₁₋₄ alkyl), —(CH₂)ₓ N(C₁₋₄ alkyl)(C₁₋₄ alkyl), —(CH₂)ₚ-cyclopropyl, —(CH₂)ₚ-cyclobutyl, or —(CH₂)ₚ-(4-6 membered heterocyclic ring having 1 or 2 ring heteroatoms independently selected from O, N, and S), wherein x is 1, 2, or 3, and p is 0, 1, 2, or 3.

In some specific embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

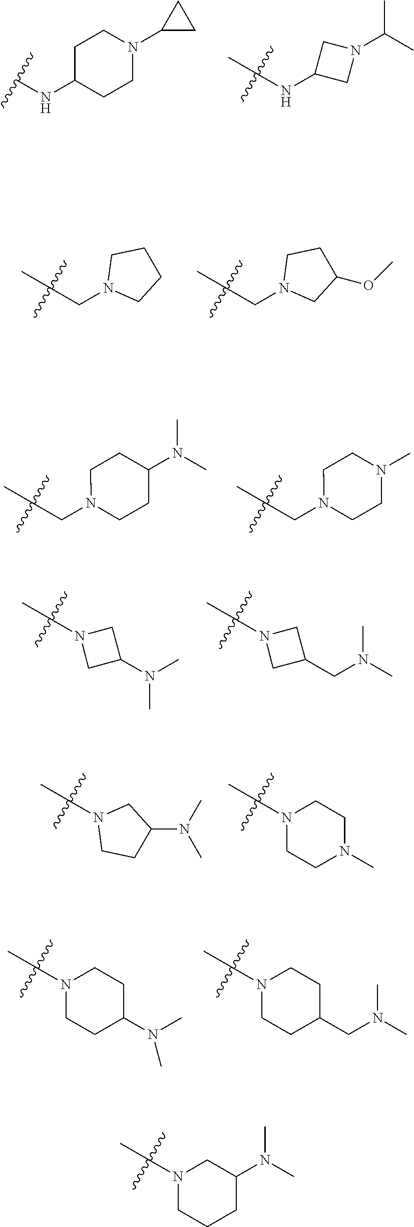

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be an optionally substituted heterocyclic or heteroaryl ring. In some embodiments, R¹ is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. In some embodiments, R¹ is an optionally substituted 4-8 membered monocyclic saturated heterocyclic ring such as those having 1 or 2 heteroatoms independently selected from O and N, preferably, the ring has at most one oxygen. In some embodiments, the 4-8 membered monocyclic saturated heterocyclic ring is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, the 4-8 membered monocyclic saturated heterocyclic ring has one ring heteroatom, which is a ring nitrogen atom (e.g., azetidine, pyrrolidine, piperazine, etc.).

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be an optionally substituted fused, bridged or spiro bicyclic 6-10 membered heterocyclic ring having one to three ring heteroatoms independently selected from N, O, and S. For example, in some embodiments, R¹ is selected from

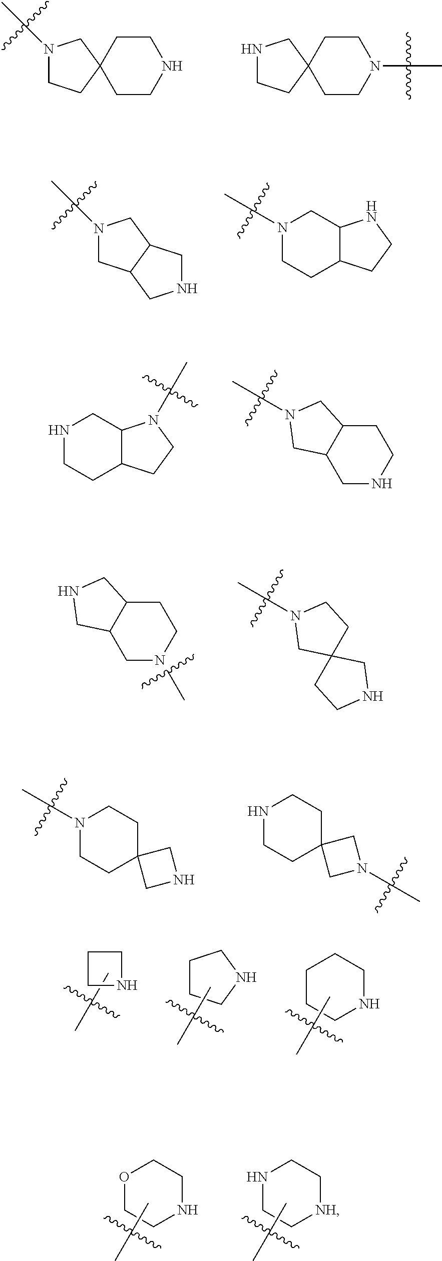

each of which is optionally substituted, for example, optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. For example, in some embodiments, R¹ can be selected from

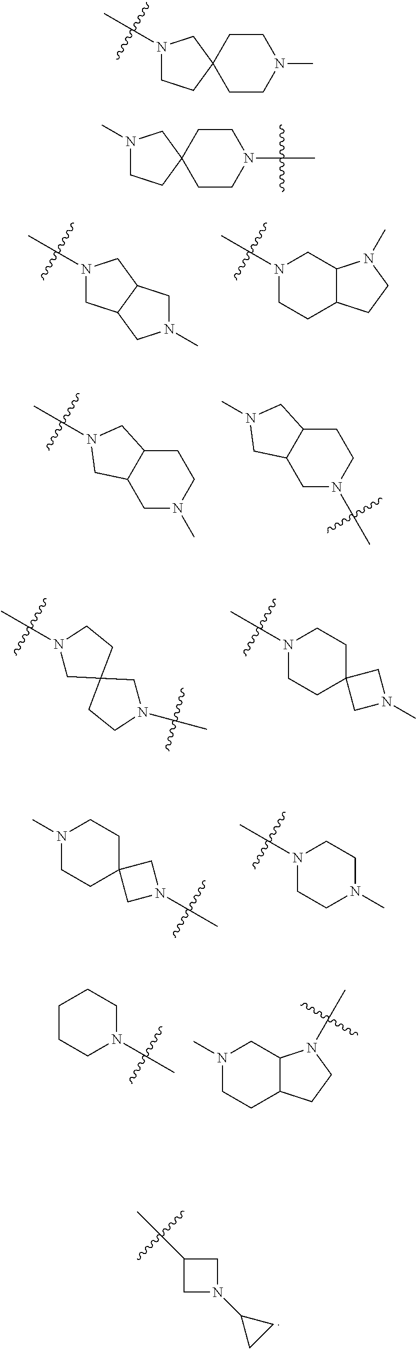

In some embodiments, R¹ can also be a bridged bicyclic structure, such as those containing 1 or 2 ring heteroatoms independently selected from nitrogen and oxygen, such as those having 1 ring nitrogen, or those having 1 ring nitrogen and 1 ring oxygen, or those having two ring nitrogens, wherein the bridged bicyclic system can be, e.g., a [2,2,1], [2,2,2], [3,1,1], or [3,2,1] bridged bicyclic system. The bridged bicyclic structure can be optionally substituted, e.g., with one or more (e.g., 1, 2 or 3) substituents independently selected from F, methyl, ethyl, isopropyl, cyclopropyl, —N(CH₃)₂, —OH, and —OCH₃.

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can have a structure of F-1:

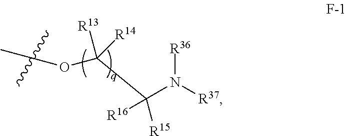

wherein:
R¹³ and R¹⁴ at each occurrence are independently hydrogen or a C₁₋₄ alkyl,
q is an integer of 0-6,
R¹, R¹⁶, R³⁶, and R³⁷, together with the intervening carbon and nitrogen atoms, form an optionally substituted 6-10 membered fused bicyclic ring.

Typically, q is 1-3. In some embodiments, q is 1. In some embodiments, q is 2. R¹³ and R¹⁴ are typically hydrogen or methyl. For example, in some embodiments, R¹³ and R¹⁴ at each occurrence are independently hydrogen or methyl. In some embodiments, R¹³ and R¹⁴ at each occurrence are hydrogen.

In some embodiments, R¹⁵, R¹⁶, R³⁶, and R³⁷, together with the intervening carbon and nitrogen atoms, form an optionally substituted 6-10 membered fused bicyclic ring selected from:

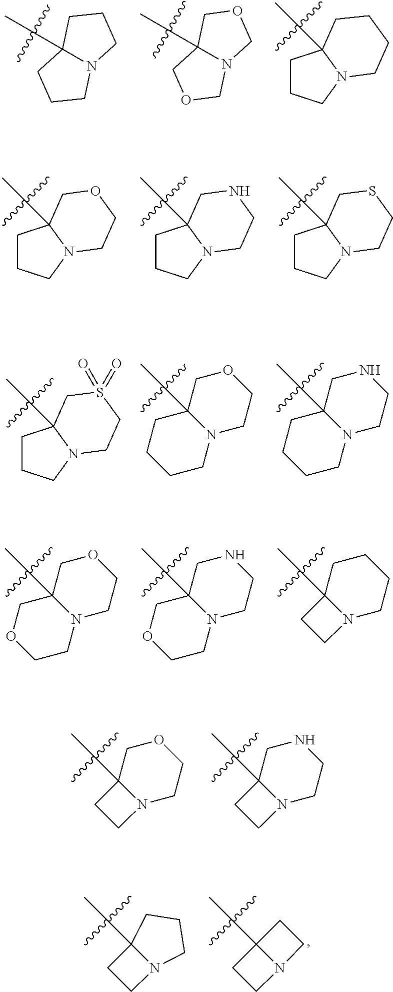

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹⁵, R¹⁶, R³⁶, and R³⁷, together with the intervening carbon and nitrogen atoms, form

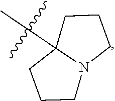 [SMILES: *C12CCCN1CCC2]

which is optionally substituted, on one or both rings. In some embodiments, the

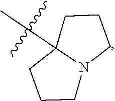 [SMILES: *C12CCCN1CCC2]

is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, only one of the pyrrolidine ring is substituted, e.g., with one fluorine.

In some specific embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is selected from

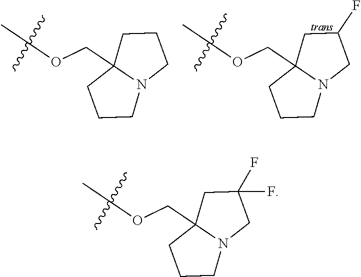 [SMILES: *OCC12CCCN1CCC2 ; *OCC12CCCN1CC(F)C2 ; *OCC12CCCN1CC(F)(F)C2]

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can have a structure of:

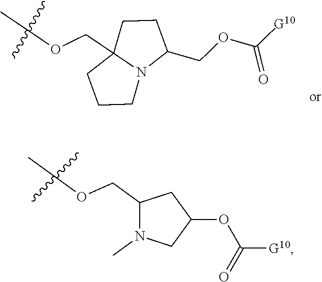

wherein G¹⁰ is amino, monoalkyl amino, dialkyl amino, or 4-10 membered heterocyclic ring, preferably, when G¹⁰ is a heterocyclic ring, the heterocyclic ring has a ring nitrogen bonded to the carbonyl group of the moiety to form a carbamate. The stereochemistry of the moiety is not particularly limited and can be any of the four possible stereoisomers or mixtures thereof in any ratio. For example, in some embodiments, R¹ can be

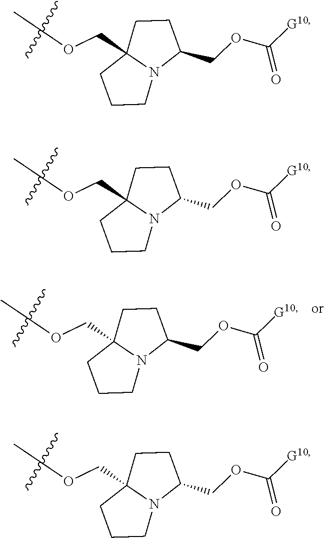

wherein G¹⁰ is defined herein. In some embodiments, R¹ can be

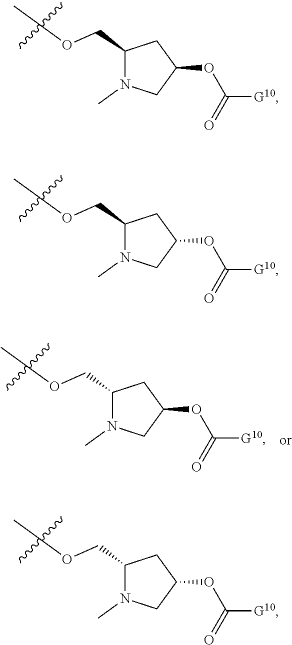

wherein G¹⁰ is defined herein. In some embodiments, G¹⁰ can be NH₂, NH(C₁₋₃₀ alkyl), or N(C₁₋₄ alkyl)(C₁₋₃₀ alkyl). In some embodiments, G¹⁰ can be NH₂, NH(C₁₋₃₀ alkyl), or N(CH₃)(C₁₋₃₀ alkyl). In some embodiments, G¹⁰ can be a 4-7 membered monocyclic heterocyclic ring having one or two ring heteroatoms independently N, O, or S. For example, in some embodiments, G¹⁰ can be

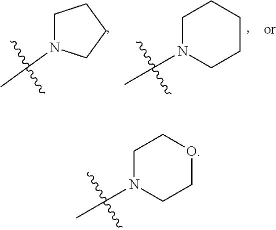 [SMILES: *N1CCCC1 ; *N1CCCCC1 ; *N1CCOCC1]

For example, in some embodiments, R¹ can be

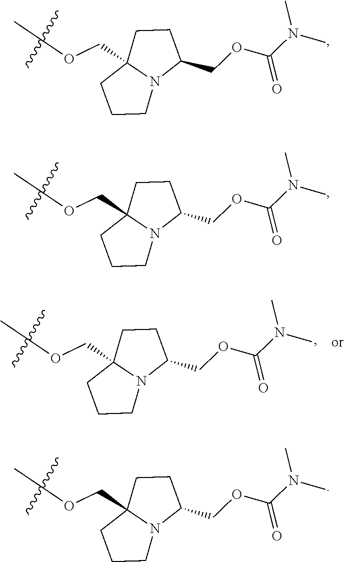 [SMILES: *OC[C@@]12CCCN1[C@H](COC(=O)N(C)C)CC2 ; *OC[C@]12CCCN1[C@@H](COC(=O)N(C)C)CC2 ; *OC[C@@]12CCCN1[C@@H](COC(=O)N(C)C)CC2 ; *OC[C@]12CCCN1[C@@H](COC(=O)N(C)C)CC2]

In some embodiments, R¹ can be

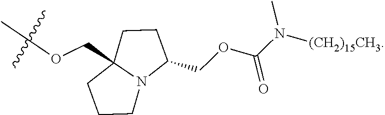

In some embodiments, R¹ can be

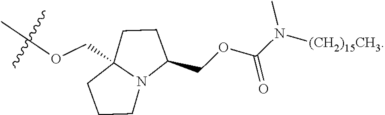

In some embodiments, R¹ can be

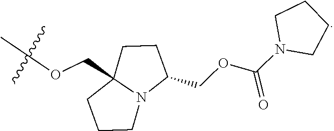 [SMILES: *OC[C@]12CCCN1[C@@H](COC(=O)N1CCCC1)CC2]

In some embodiments, R¹ can be

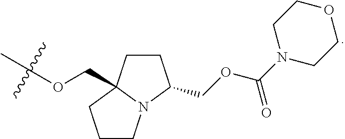 [SMILES: *OC[C@]12CCCN1[C@@H](COC(=O)N1CCOCC1)CC2]

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also have a structure of:

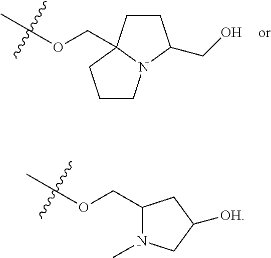

The stereochemistry of the moiety is not particularly limited and can be any of the four possible stereoisomers or mixtures thereof in any ratio. For example, in some embodiments, R¹ can be

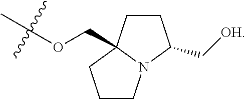 [SMILES: *OC[C@]12CCCN1[C@@H](CO)CC2]

In some embodiments, R¹ can be

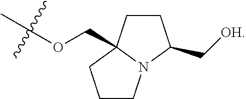 [SMILES: *OC[C@]12CCCN1[C@H](CO)CC2]

In some embodiments, R¹ can be

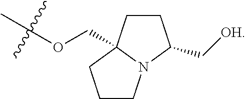 [SMILES: *OC[C@@]12CCCN1[C@@H](CO)CC2]

In some embodiments, R¹ can be

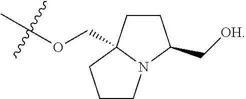 [SMILES: *OC[C@@]12CCCN1[C@H](CO)CC2]

In some embodiments, R¹ can b

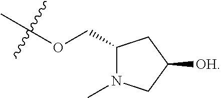 [SMILES: *OC[C@@H]1C[C@@H](O)CN1C]

In some embodiments, R¹ can be

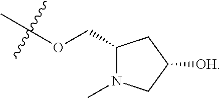 [SMILES: *OC[C@@H]1C[C@H](O)CN1C]

In some embodiments, R¹ can be

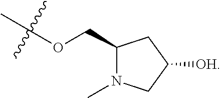 [SMILES: *OC[C@H]1C[C@H](O)CN1C]

In some embodiments, R¹ can be

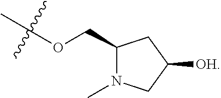 [SMILES: *OC[C@H]1C[C@@H](O)CN1C]

In some specific embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can selected from:

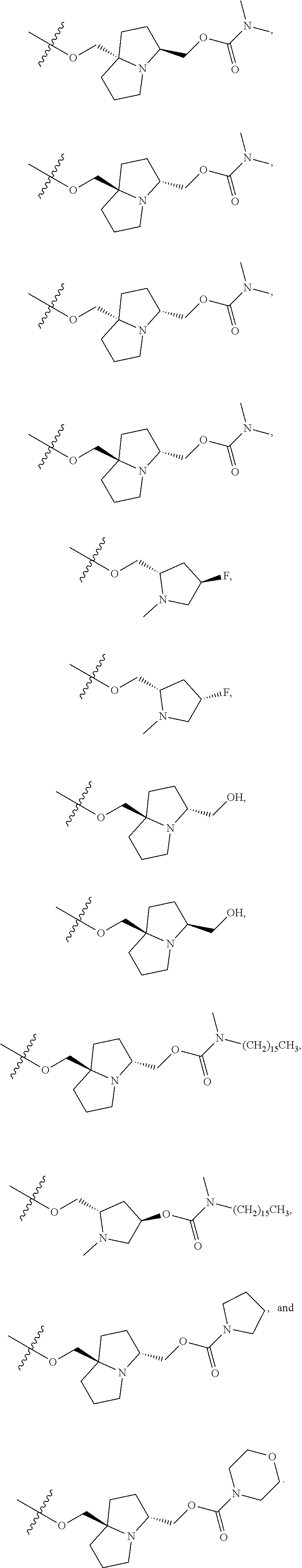

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be (1) C₁₋₆ alkoxy optionally substituted with 1-3 F, such as methoxy, (2) hydroxyl substituted C₁₋₆ alkoxy, such as hydroxyl ethoxy, (3) alkoxy substituted C₁₋₆ alkoxy, such as methoxy ethoxy, or (4) amino or alkyl amino substituted C₁₋₆ alkoxy, such as N,N-dimethylamino ethoxy. For example, in some embodiments, R¹ in Formula I can be methoxy,

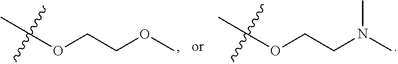

In some embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can also be NH₂, NH(C₁₋₆ alkyl), or N(C₁₋₆ alkyl)(C₁₋₆ alkyl). For example, in some embodiments, R¹ in Formula I can be NH₂, NH(CH₃), or N(CH₃)₂.

In some specific embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

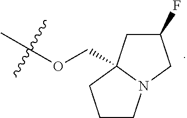 [SMILES: *OC[C@@]12CCCN1C[C@H](F)C2]

In some specific embodiments, R¹ in Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

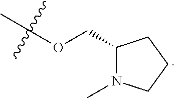 [SMILES: *OC[C@@H]1CCCN1C]

In some specific embodiments, R¹ in Formula I is such that the compounds of Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can have one of the following formulae:

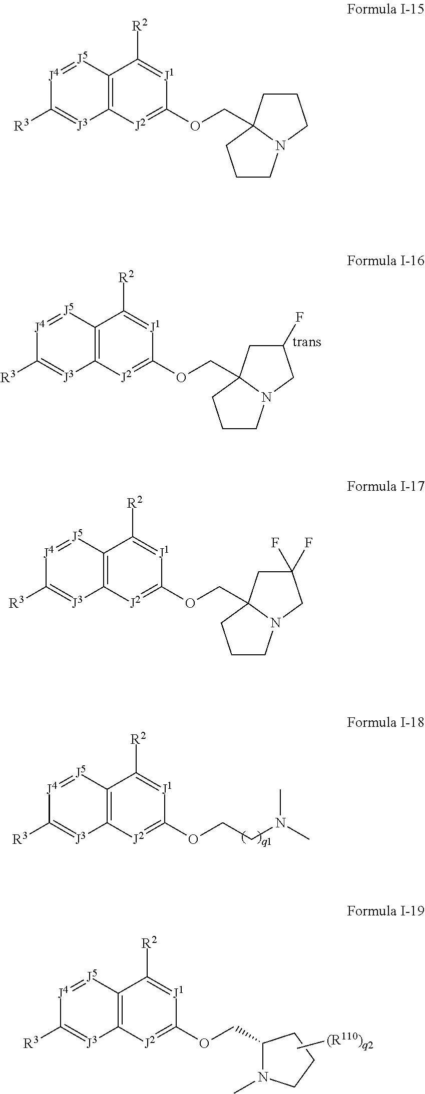

wherein q1 is 1 or 2, q2 is 0, 1, or 2, R¹¹⁰ at each occurrence is independently F or hydroxyl; and wherein J¹, J², J³, J⁴, J⁵, R², and R³ include any of those defined herein, including those specified in the sub-formulae of Formula I (e.g., sub-formulae I-1, I-2, I-3, I-4, I-5, I-6, I-7, I-8, I-9, I-10, I-11, I-12, I-13, I-14, I-23, I-24, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B). In some embodiments, q2 in Formula I-19 is 0. In some embodiments q2 in Formula I-19 is 1, and R¹¹⁰ is F or hydroxyl. The “trans” designation in Formula I-16 indicates that the F substitution is trans to the ether-linked moiety. For the avoidance of doubt, Formula I-16 includes individual stereoisomers (enantiomers etc.) and mixtures of stereoisomers in any ratio (including racemic mixtures). In some embodiments, the compound of Formula I-16 can have a formula according to I-16-E1 or I-16-E2:

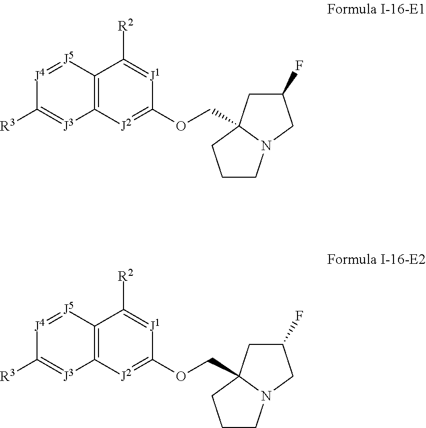

wherein J¹, J², J³, J⁴, J⁵, R², and R³, include any of those defined herein, including those specified in the sub-formulae of Formula I. In some embodiments, compounds of Formula I-16-E1 or I-16-E2 can exist predominantly as the as-drawn enantiomer (with respect to the two chiral centers showing stereochemical drawings), such as with less than 20%, less than 10%, less than 5%, less than 1%, by weight, by HPLC area, or both, or with a non-detectable amount of the other enantiomer. The enantiomers can be typically separated through chiral HPLC, e.g., as exemplified herein.

Various groups are suitable as R² for Formula I, which include any of those exemplified in the specific compounds herein. Typically, R² in Formula I does not contain a Michael acceptor, such as an alpha-beta unsaturated carbonyl structural moiety. In some embodiments, R² can be represented by -(L²)ₘ₂-R¹⁰², wherein m2 is 0-3, typically 0 or 1, and when m2 is not 0, for example, m2 is 1, L² at each occurrence is independently CH₂, O, NH, or NCH₃, R¹⁰² is an optionally substituted 4-10 membered heterocyclic ring or a heteroaryl ring, e.g., those heterocyclic or heteroaryl rings having one or two ring nitrogen atoms. To be clear, when it is said that the heterocyclic or heteroaryl rings have one or two ring nitrogen atoms, the heterocyclic or heteroaryl rings may contain additional ring heteroatoms such as ring oxygen or ring sulfur atom(s). However, in some embodiments, the heterocyclic or heteroaryl rings only have the ring nitrogen atoms as ring heteroatoms. In some embodiments, m2 is 0. In some embodiments, m2 is 1.

In some embodiments, m2 is 0, and R¹⁰² is an optionally substituted 4-10 membered heterocyclic ring having one or two ring nitrogen atoms. For example, in some embodiments, R¹⁰² is selected from the following ring structures:

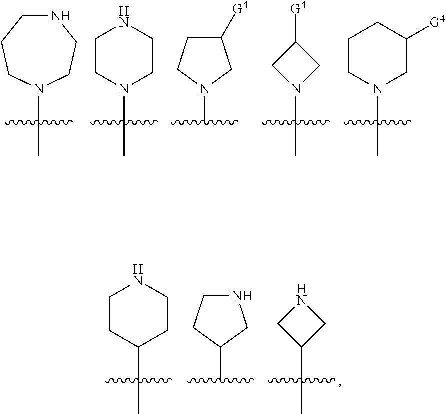

each of which is optionally substituted,
wherein G⁴ is -(L³)ₘ₃-NH₂, -(L³)ₘ₃-NH(C₁₋₄ alkyl), wherein m3 is 0 or 1, and when m3 is 1, L³ is C₁₋₄ alkylene (e.g., methylene, ethylene, propylene, isopropylene, etc.),
or G⁴ and one substituent on the ring are joined together to form a 4-6 membered heterocyclic ring having one or two ring nitrogen atoms. In some embodiments, each of the ring structures drawn above is optionally substituted with 1-3 (typically 1 or 2) substituents independently selected from C₁₋₄ alkyl (e.g., methyl, ethyl, etc.), fluorine substituted C₁₋₄ alkyl (e.g., CHF₂, CH₂F, or CF₃), hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure (preferably a 3-5 membered ring such as cyclopropyl or cyclobutyl ring). The substitution can occur on any available position of the rings, including the ring nitrogen atoms.

In some embodiments, R¹⁰² or R² in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is selected from:

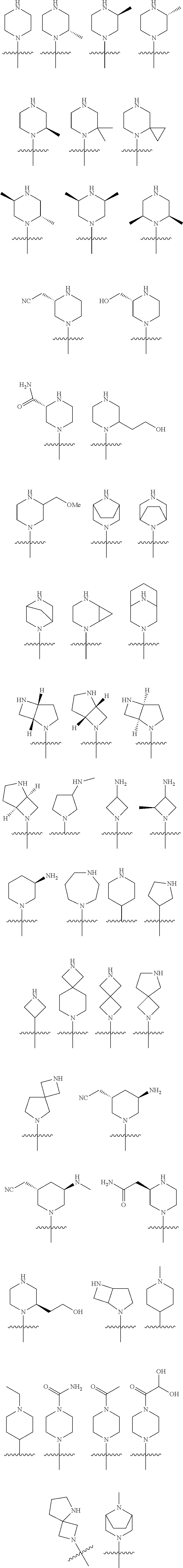

or selected from

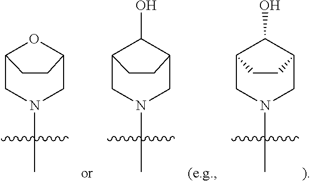 [SMILES: *N1CC2CCC(C1)O2 ; *N1CC2CCC(C1)C2O ; *N1C[C@H]2CC[C@@H](C1)[C@@H]2O]

or selected from

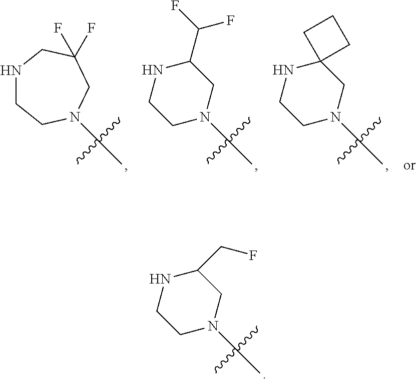 [SMILES: *N1CCNCC(F)(F)C1 ; *N1CCNC(C(F)F)C1 ; *N1CCNC2(CCC2)C1 ; *N1CCNC(CF)C1]

In some embodiments, m2 is 1, L is CH₂ or NH, and R¹⁰² is an optionally substituted 4-10 membered heterocyclic ring having one or two ring nitrogen atoms. For example, in some embodiments, m2 is 1, L² is CH₂ or NH, and R¹⁰² is an optionally substituted 4-8 membered heterocyclic ring, e.g., a monocyclic saturated 4-8 membered ring, which is optionally substituted. For example, in some embodiments, m2 is 1, L² is CH₂ or NH, and R¹⁰² is selected from:

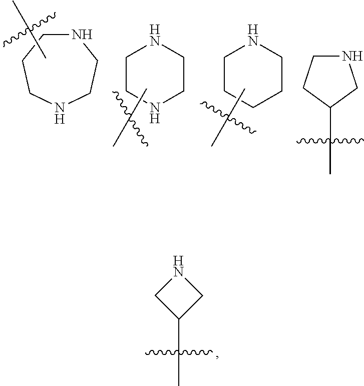

each of which is optionally substituted, for example, optionally substituted with 1-3 (typically 1 or 2) substituents independently selected from C₁₋₄ alkyl (e.g., methyl, ethyl, etc.), fluorine substituted C₁₋₄ alkyl (e.g., CF₃), hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure. The substitution can occur on any available position of the rings, including the ring nitrogen atoms.

In some embodiments, in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), R² is selected from:

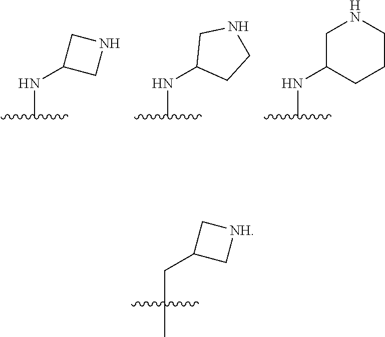 [SMILES: *NC1CNC1 ; *NC1CCNC1 ; *NC1CCCNC1 ; *CC1CNC1]

In some embodiments, in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), R² can also be -(L²)ₘ₂-R¹⁰², wherein m2 is 0 or 1, and when m2 is 1, L² is CH₂, O, NH, or NCH₃, wherein R¹⁰² is an optionally substituted C₃₋₇ carbocyclic (e.g., cyclopropyl, cyclobutyl, cyclopentyl, cyclohexyl, etc.), optionally substituted phenyl, or optionally substituted 5 or 6 membered heteroaryl ring, each of which has at least one nitrogen containing substituent, e.g., NH₂, NH(C₁₋₄ alkyl) or N(C₁₋₄ alkyl)(C₁₋₄ alkyl). In some embodiments, m2 is 1. In some embodiments, m2 is 0, and R² can be a C₃₋₇ carbocyclic, phenyl, or 5 or 6 membered heteroaryl ring, each of which has at least one nitrogen containing substituent, e.g., a basic nitrogen containing substituent, such as NH₂, NH(C₁₋₄ alkyl), or NH(C₁₋₄ alkyl)(C₁₋₄ alkyl). For example, in some embodiments, R² is selected from

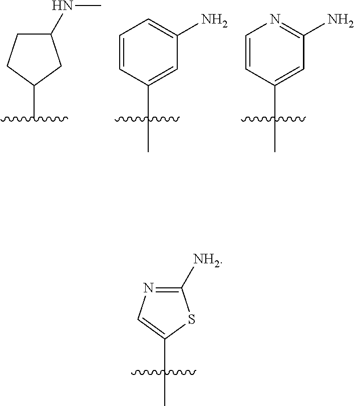

In some embodiments, in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), R² can have a structure of F-2

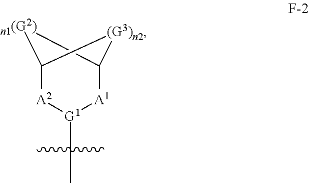

wherein:
G1 is CR¹⁷ or N;
each occurrence of G² and G³ is independently CR¹⁸R¹⁹, O, or NR³⁸, provided that at least one instance of G² and G³ is NR³⁸;
n1 and n2 are each independently an integer of 1, 2, 3, or 4;
A¹ and A² are each independently a bond, CR¹⁸R¹⁹, O, or NR³⁸, provided that at least one of A¹ and A² is not O or NR³⁸,
wherein: each occurrence of R¹⁷, R¹⁸, or R¹⁹ is independently hydrogen, F, —OH, or an optionally substituted C₁₋₆ alkyl, or R¹⁸ and R¹⁹ together with the carbon they are both attached to are joined to form an oxo or imino group or a ring; and
R³⁸ at each occurrence is independently hydrogen, a nitrogen protecting group, or an optionally substituted C₁₋₆ alkyl.

In some embodiments, G¹ in F-2 is N.

In some embodiments, G¹ in F-2 is CR¹⁷. In some embodiments, R¹⁷ can be hydrogen, F, —OH, or C₁₋₆ alkyl (such as methyl, ethyl, etc.) which can be optionally substituted, for example, with F, —OH, methoxy, etc. Typically, when G¹ is CR¹⁷, R¹⁷ is hydrogen.

A¹ and A² in F-2 can independently be a bond, a carbon-based linker, oxygen, or a nitrogen-based linker. Typically, A¹ and A² in F-2 can independently be a bond or CR¹⁸R¹⁹. In some embodiments, one of A¹ and A² is a bond. In some embodiments, both A¹ and A² are a bond, thus, both of the bridging points are directly connected to G¹. In some embodiments, one of A¹ and A² is CR¹⁸R¹⁹, wherein R¹⁸ and R¹⁹ can be independently hydrogen, F, —OH, or C₁₋₆ alkyl (such as methyl, ethyl, etc.) which can be optionally substituted, for example, with F, —OH, methoxy, etc. In some embodiments, one of A¹ and A² is CR¹⁸R¹⁹, wherein R¹⁸ and R¹⁹ together with the carbon they are both attached to are joined to form an oxo or imino group or a ring (e.g., cyclopropyl), for example, A¹ can be C═O, C═NH, etc. In some embodiments, both A¹ and A² are independently selected CR¹⁸R¹⁹, wherein R¹⁸ and R¹⁹ are defined herein. For example, in some embodiments, both A¹ and A² are CH₂. In some embodiments, one of A¹ and A² is CH₂ and the other of A¹ and A² is C═O or C═NH. In some embodiments, both A¹ and A² are C═O.

In some embodiments, each occurrence of G² in F-2 can be independently CR¹⁸R¹⁹. In such embodiments, at least one instance of G³ is NR³⁸. In some embodiments, each occurrence of G² can be the same. In some embodiments, each occurrence of G² can also be different from each other, or some of the G² are the same whereas others are different. In some embodiments, each occurrence of G² can be independently CR¹⁸R¹⁹, wherein R¹⁸ and R¹⁹ can be independently hydrogen, F, —OH, or C₁₋₆ alkyl (such as methyl, ethyl, etc.) which can be optionally substituted, for example, with F, —OH, methoxy, etc. In some embodiments, one or two instances of G² can be CR¹⁸R¹⁹, wherein R¹⁸ and R¹⁹ together with the carbon they are both attached to are joined to form an oxo or imino group or a ring (e.g., cyclopropyl). For example, in some embodiments, one instance of G² can be C═O or C═NH.

In some embodiments, one or two instances of G² can be O or NR³⁸. Typically, at most one of G² is a heteroatom based moiety, such as O or NR³⁸, and the other instances of G² are independently CR¹⁸R¹⁹.

In some embodiments, each occurrence of G³ in F-2 can be independently CR¹⁸R¹⁹. In such embodiments, at least one instance of G² is NR³⁸. In some embodiments, each occurrence of G³ can be the same. In some embodiments, each occurrence of G³ can also be different from each other, or some of the G³ are the same whereas others are different. In some embodiments, each occurrence of G³ can be independently CR¹⁸R¹⁹, wherein R¹⁸ and R¹⁹ can be independently hydrogen, F, —OH, or C₁₋₆ alkyl (such as methyl, ethyl, etc.) which can be optionally substituted, for example, with F, —OH, methoxy, etc. In some embodiments, one or two instances of G³ can be CR¹⁸R¹⁹, wherein R¹⁸ and R¹⁹ together with the carbon they are both attached to are joined to form an oxo or imino group or a ring (e.g., cyclopropyl). For example, in some embodiments, one instance of G³ can be C═O or C═NH.

In some embodiments, one or two instances of G³ can be O or NR³⁸. Typically, at most one of G³ is a heteroatom based moiety, such as O or NR³⁸, and the other instances of G³ are independently CR¹⁸R¹⁹.

Typically, F-2 includes 1, 2, or 3 G² (as defined herein), i.e., n1 is 1, 2 or 3. In some embodiments, F-2 includes 1, 2, or 3 G³ (as defined herein), i.e., n2 is 1, 2 or 3.

As described herein, at least one instance out of all G² and G³ is NR³⁸. In some embodiments, one instance out of all G² and G³, i.e., one G2 or one G3 among all G² and G³, is NR³⁸. For example, in some embodiments, among all G² and G³, one G2 or one G³ is NR 3, wherein R³⁸ is hydrogen or C₁₋₄ alkyl (e.g., methyl). In some embodiments, R³⁸ at each occurrence can be independently hydrogen, a nitrogen protecting group (e.g., described herein), or a C₁₋₆ alkyl (e.g., methyl, ethyl, isopropyl, etc.), which can be optionally substituted, for example, with 1, 2, or 3 substituents independently selected from F, —OH, protected hydroxyl, oxo, NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, cyclopropyl, fluoro-substituted C₁₋₄ alkyl (e.g., CF₃), C₁₋₄ alkoxy, and fluoro-substituted C₁₋₄ alkoxy.

In some embodiments, the compound of Formula I can be characterized as having Formula I-20, I-21, or I-22:

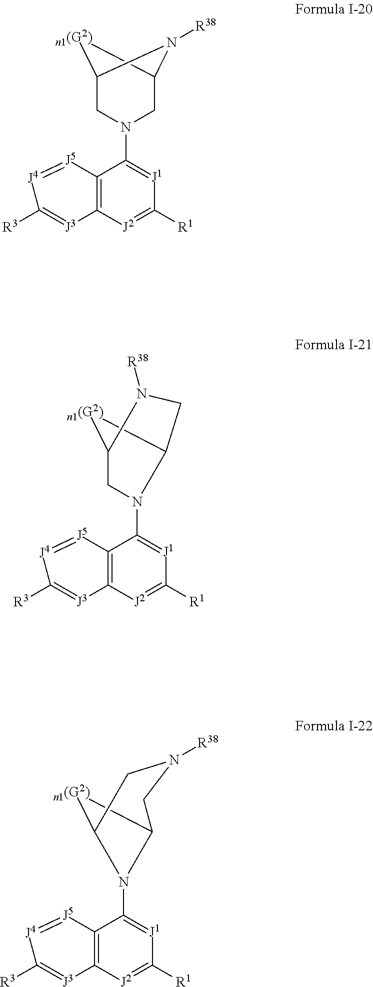

wherein the variables are defined herein J¹, J², J³, J⁴, J⁵, R¹, R³, R³⁸, G², and n1 include any of those defined herein, including those specified in the sub-formulae of Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B). For example, in some embodiments, n1 is 1, 2, or 3, and each G² can be CH₂. In some embodiments, R³⁸ can be hydrogen.

In some specific embodiments, R² in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E11, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is selected from the following:

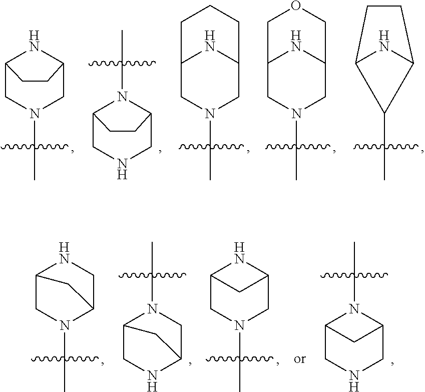

In some embodiments, R² in Formula I can also be

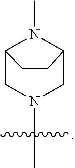 [SMILES: *N1CC2CCC(C1)N2C]

In some specific embodiments, R² in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is selected from the following:

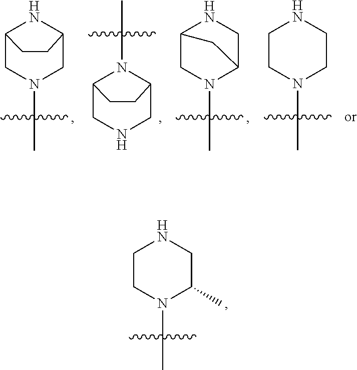

preferably,

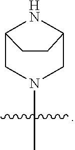 [SMILES: *N1CC2CCC(C1)N2]

In some specific embodiments, R² in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is

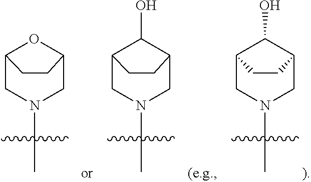 [SMILES: *N1CC2CCC(C1)O2 ; *N1CC2CCC(C1)C2O ; *N1C[C@H]2CC[C@@H](C1)[C@@H]2O]

In some specific embodiments, R² in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) is

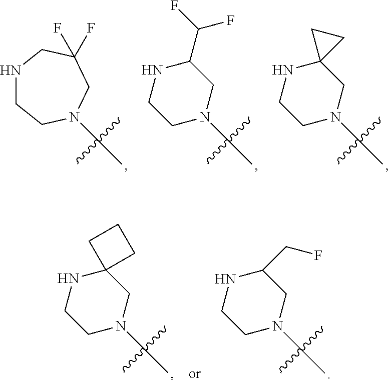 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

Various groups suitable for R³ in Formula I are described herein. In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be a phenyl or 5 or 6 membered heteroaryl, such as pyridyl, which is optionally substituted. In some embodiments, R³ is a phenyl substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, R³ is a pyridyl substituted with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, at most one of the substituents is OH, —NH₂, protected —OH, or a protected —NH₂. For example, in some embodiments, R³ can be

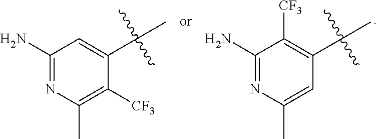 [SMILES: *c1cc(N)nc(C)c1C(F)(F)F ; *c1cc(C)nc(N)c1C(F)(F)F]

In some embodiments, R³ can be

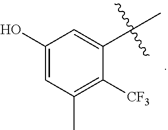 [SMILES: *c1cc(O)cc(C)c1C(F)(F)F]

In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be a naphthyl, which is optionally substituted, for example, with one or more (typically, 1-3) substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, at most one of the substituents is OH, —NH₂, protected —OH, or a protected —NH₂. In some embodiments, R³ is

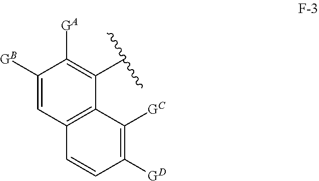

wherein:
1) GB is OH, G^{A} is H, and G^{C} and GD are independently H, F, Cl, CN, C₁₋₄ alkyl optionally substituted with 1-3 fluorine, such as methyl, ethyl, or CF₃, preferably, G^{D} is H, F, or methyl;
2) G^{C} is Cl, methyl, ethyl, ethynyl, or CN, G^{A} is H, GB is H or OH, and GD is H, F, Cl, CN, C₁₋₄ alkyl optionally substituted with 1-3 fluorine, such as methyl, ethyl, or CF₃, preferably, G^{D} is H, F, or methyl; or
3) G^{A} is Cl, GB is H, F, or methyl, G^{C} and GD are independently H, F, Cl, CN, C₁₋₄ alkyl optionally substituted with 1-3 fluorine, such as methyl, ethyl, or CF₃, preferably, G^{C} and GD are independently H, F, or methyl.

In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be an optionally substituted naphthyl, such as a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, at most one of the substituents is OH, —NH₂, protected —OH, or a protected —NH₂. In some embodiments, R³ is

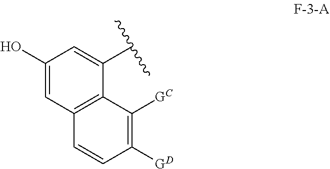

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl or propargyl), preferably, G^{D} is H, F, or methyl. In some embodiments, in F-3-A, G^{C} is Cl, methyl, ethyl, ethynyl, or CN, and G^{D} is H, F, Cl, CN, C₁₋₄ alkyl optionally substituted with 1-3 fluorine, such as methyl, ethyl, or CF₃. In some embodiments, in F-3-A, G^{C} is Cl, methyl, ethyl, ethynyl, or CN, and G^{D} is H or F. In some embodiments, R³ is

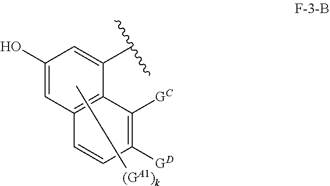

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl or propargyl), preferably, G^{D} is H, F, or methyl, wherein G^{A1} at each occurrence is independently a halo (e.g., F, or Cl), OH, CN, cyclopropyl, optionally substituted C₁₋₄ alkyl, or optionally substituted C₁₋₄ alkoxy, and k is 1, 2, or 3. It should be noted that the G^{A1} in F-3-B can be substituted at any available position of the naphthyl ring, although preferably, one or two G^{A1} is/are ortho to the OH group. In some embodiments, in F-3-B, G^{C} is Cl, methyl, ethyl, ethynyl, or CN, and G^{D} is H, F, Cl, CN, C₁₋₄ alkyl optionally substituted with 1-3 fluorine, such as methyl, ethyl, or CF₃. In some embodiments, in F-3-B, G^{C} is Cl, methyl, ethyl, ethynyl, or CN, and G^{D} is H or F. In some embodiments, k is 1, G^{A1} is ortho to the OH group, and G^{A1} is F, Cl, CN, or C₁₋₄ alkyl optionally substituted with 1-3 fluorine. In some embodiments, k is 2, both G^{A1} are ortho to the OH group, and each G^{A1} is independently F, Cl, CN, or C₁₋₄ alkyl optionally substituted with 1-3 fluorine.

In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be an optionally substituted naphthyl, such as a naphthyl optionally substituted with one or more (typically, 1-4, more typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), optionally substituted C₁₋₄ alkoxy (e.g., methoxy, ethoxy, etc.), optionally substituted C₃₋₅ cycloalkyl, such as cyclopropyl, optionally substituted C₃₋₅ cycloalkoxy, —NH₂, —CN, protected —OH, and a protected —NH₂.

In some embodiments, R³ in Formula I is

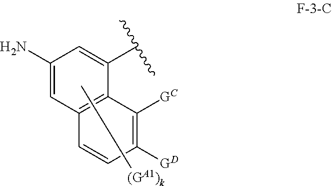

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl or propargyl), preferably, G^{D} is H, F, or methyl, wherein G^{A1} at each occurrence is independently a halo (e.g., F, or Cl), OH, CN, cyclopropyl, optionally substituted C₁₋₄ alkyl, or optionally substituted C₁₋₄ alkoxy, and k is 0, 1, 2, or 3. It should be noted that when present, the G^{A1} in F-3-C can be substituted at any available position of the naphthyl ring, although preferably, one or two G^{A1} is/are ortho to the NH2 group. In some embodiments, in F-3-C, G^{C} is Cl, methyl, ethyl, ethynyl, propargyl, or CN, and G^{D} is H, F, Cl, CN, C₁₋₄ alkyl optionally substituted with 1-3 fluorine, such as methyl, ethyl, or CF₃. In some embodiments, in F-3-C, G^{C} is Cl, methyl, ethyl, ethynyl, or CN, and G^{D} is H or F. In some embodiments, k is 0. In some embodiments, k is 1, G^{A1} is ortho to the NH₂ group, and G^{A1} is F, Cl, CN, or C₁₋₄ alkyl optionally substituted with 1-3 fluorine. In some embodiments, k is 2, both G^{A1} are ortho to the NH₂ group, and each G^{A1} is independently F, Cl, CN, or C₁₋₄ alkyl optionally substituted with 1-3 fluorine.

In some embodiments, R³ in Formula I is

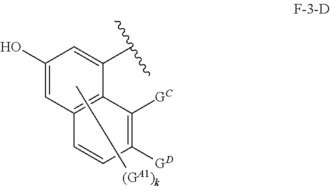

wherein G^{C} and G^{D} are independently H, F, Cl, CN, C₁₋₄ alkyl optionally substituted with 1-3 fluorine, such as methyl, ethyl, or CF₃, C₁₋₄ alkoxy optionally substituted with 1-3 fluorine (e.g., methoxy, ethoxy, difluoromethoxy, etc.), cyclopropyl, or C₂₋₄ alkynyl (e.g., ethynyl or propargyl), preferably, G^{D} is H, F, or methyl, wherein G^{A1} at each occurrence is independently a halo (e.g., F, or Cl), OH, CN, cyclopropyl, optionally substituted C₁₋₄ alkyl, or optionally substituted C₁₋₄ alkoxy, and k is 0, 1, 2, or 3. It should be noted that when present, the G^{A1} in F-3-D can be substituted at any available position of the naphthyl ring, although preferably, one or two G^{A1} is/are ortho to the OH group. In some embodiments, in F-3-D, G^{C} is Cl, methyl, ethyl, methoxy, ethoxy, difluoromethoxy, ethynyl, propargyl, or CN, and G^{D} is H, F, Cl, CN, C₁₋₄ alkyl optionally substituted with 1-3 fluorine, such as methyl, ethyl, or CF₃. In some embodiments, in F-3-D, G^{C} is Cl, methyl, ethyl, ethynyl, or CN, and G^{D} is H or F. In some embodiments, k is 0. In some embodiments, k is 1, G^{A1} is ortho to the OH group, and G^{A1} is F, Cl, CN, or C₁₋₄ alkyl optionally substituted with 1-3 fluorine. In some embodiments, k is 2, both G^{A} are ortho to the OH group, and each G^{A1} is independently F, Cl, CN, or C₁₋₄ alkyl optionally substituted with 1-3 fluorine.

In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be a bicyclic heteroaryl (e.g., benzothiazolyl, indazolyl, or isoquinolinyl), which is optionally substituted, for example, with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, at most one of the substituents is OH, —NH₂, protected —OH, or a protected —NH₂. For example, in some embodiments, R³ is

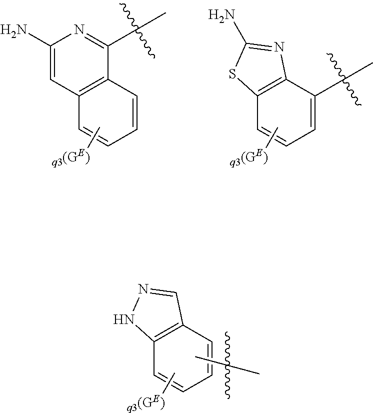

wherein: q3 is 0, 1, or 2, and G^{E} at each occurrence is independently F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, q3 is 0, 1, or 2, and G^{E} at each occurrence is F, Cl, C₁₋₄ alkyl (e.g., methyl, ethyl, propyl, isopropyl, tert-butyl), C₂₋₄ alkenyl, C₂₋₄ alkynyl (e.g., ethynyl or propargyl), cyclopropyl, CH₂CH₂—CN, CF₂H, CF₃, or —CN.

Suitable R³ for Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) also includes any of those exemplified herein in the specific examples. In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be selected from:

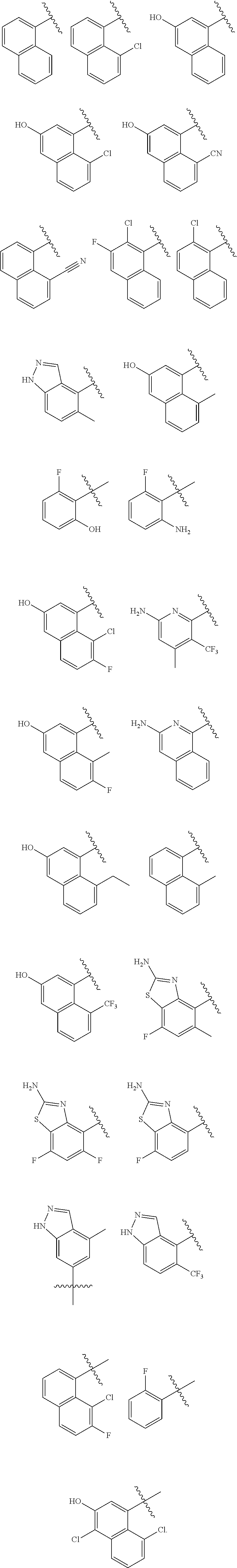

In some embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be selected from:

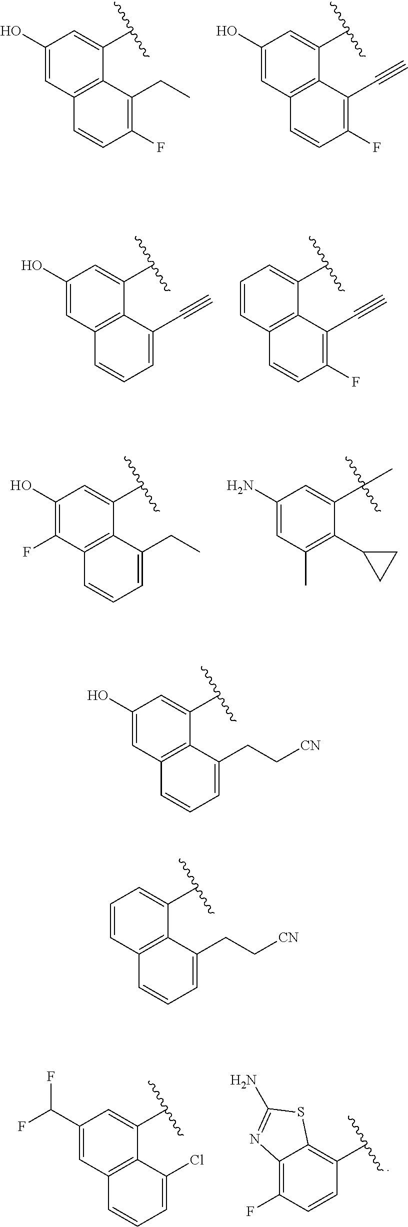

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be selected from:

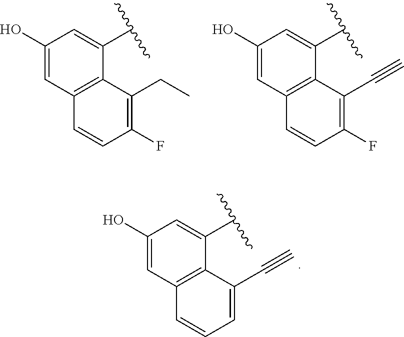 [SMILES: *c1cc(O)cc2ccc(F)c(CC)c12 ; *c1cc(O)cc2ccc(F)c(C#C)c12 ; *c1cc(O)cc2cccc(C#C)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

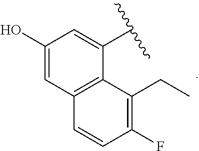 [SMILES: *c1cc(O)cc2ccc(F)c(CC)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

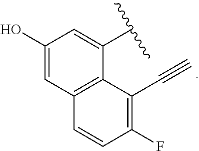 [SMILES: *c1cc(O)cc2ccc(F)c(C#C)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

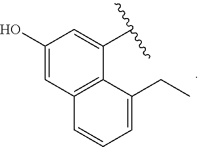 [SMILES: *c1cc(O)cc2cccc(CC)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

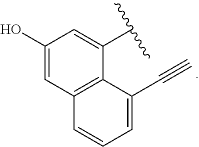 [SMILES: *c1cc(O)cc2cccc(C#C)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

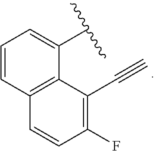 [SMILES: *c1cccc2ccc(F)c(C#C)c12]

In some preferred embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

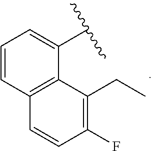 [SMILES: *c1cccc2ccc(F)c(CC)c12]

In some specific embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

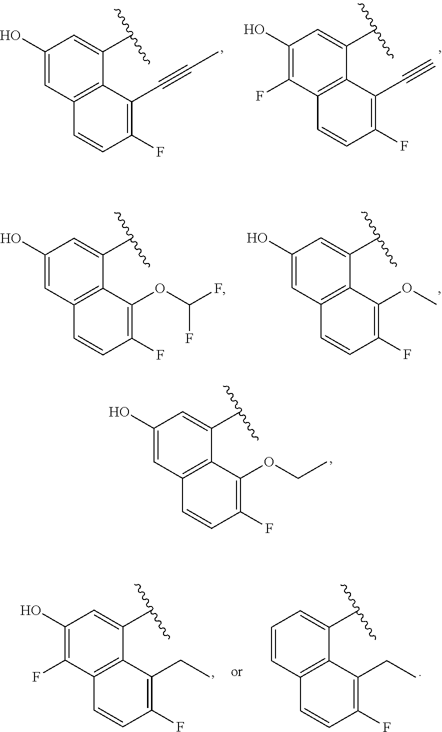

In some specific embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

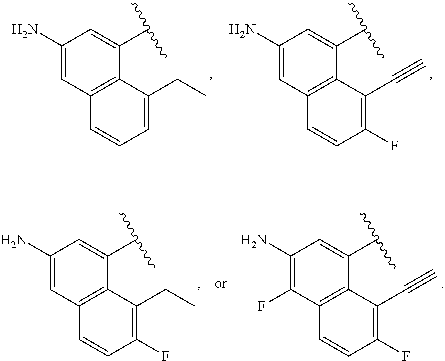 [SMILES: *c1cc(N)cc2cccc(CC)c12 ; *c1cc(N)cc2ccc(F)c(C#C)c12 ; *c1cc(N)cc2ccc(F)c(CC)c12 ; *c1cc(N)c(F)c2ccc(F)c(C#C)c12]

In some specific embodiments, R³ in Formula I (e.g., sub-formulae I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B) can be

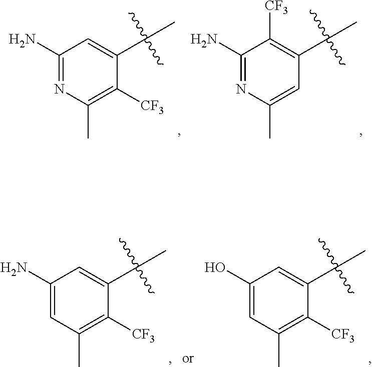 [SMILES: *c1cc(N)nc(C)c1C(F)(F)F ; *c1cc(C)nc(N)c1C(F)(F)F ; *c1cc(N)cc(C)c1C(F)(F)F ; *c1cc(O)cc(C)c1C(F)(F)F]

In some embodiments, the present disclosure provides the following exemplary Embodiments 1-57:
Embodiment 1. A compound according to Formula I-9:

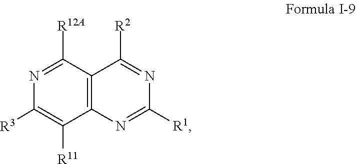

or a pharmaceutically acceptable salt thereof,
wherein the variables R¹, R², R³, R¹¹, and R^{12A} include any of those described herein in connection with Formula I-9 in any combination.
Embodiment 2. The compound of Embodiment 1, or a pharmaceutically acceptable salt thereof, wherein R¹¹ is F.
Embodiment 3. The compound of Embodiment 1 or 2, or a pharmaceutically acceptable salt thereof, wherein R^{12A} is H.
Embodiment 4. The compound of Embodiment 1 or 2, or a pharmaceutically acceptable salt thereof, wherein R^{12A} is halogen, —OH, C₁₋₄ alkyl optionally substituted with 1-3 F, C₁₋₄ alkoxy optionally substituted with 1-3 F, or cyclopropyl substituted C₁₋₄ alkoxy, which is optionally substituted 1-3 F.
Embodiment 5. The compound of Embodiment 1 or 2, or a pharmaceutically acceptable salt thereof, wherein R^{12A} is Cl, —OH, methoxy, difluoromethoxy, ethoxy, isopropoxy, —O—CH₂-cyclopropyl, —O—CH₂—CH₂-cyclopropyl, —C(O)NHMe, —O—CH₂—C(O)NHMe, —O—CH₂—CF₃, —O—CH₂—CHF₂, methyl, CHF₂, CF₃, ethyl, isopropyl, or cyclopropyl.
Embodiment 6. The compound of Embodiment 1 or 2, or a pharmaceutically acceptable salt thereof, wherein Formula I-9 is characterized as having one of the following subformulae:

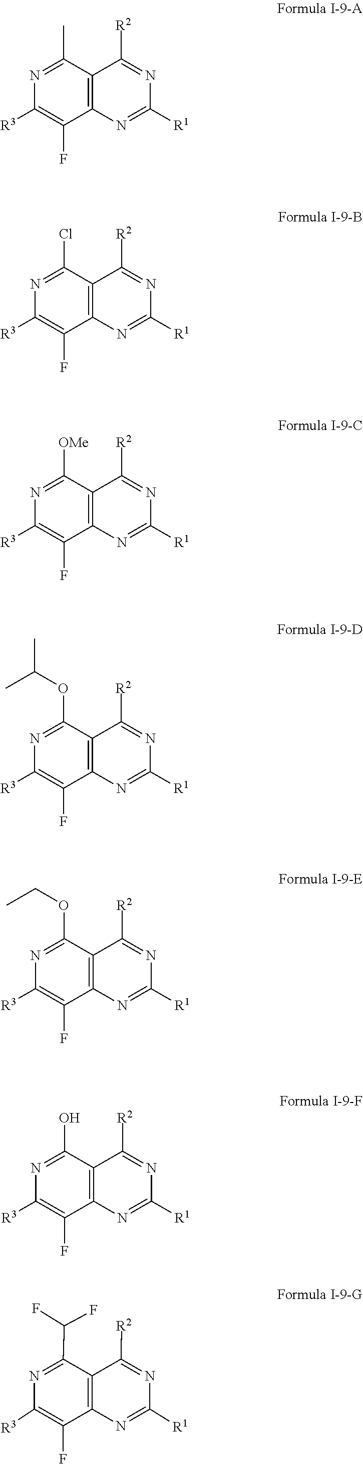

Embodiment 7. The compound of any one of Embodiments 1-6, or a pharmaceutically acceptable salt thereof, wherein R¹ is H.
Embodiment 8. The compound of any one of Embodiments 1-6, or a pharmaceutically acceptable salt thereof, wherein R¹ is C₁₋₄ alkyl optionally substituted with 1-3 F.
Embodiment 9. The compound of any one of Embodiments 1-6, or a pharmaceutically acceptable salt thereof, wherein R¹ is methyl or CHF₂.
Embodiment 10. The compound of any one of Embodiments 1-6, or a pharmaceutically acceptable salt thereof, wherein R¹ is selected from:

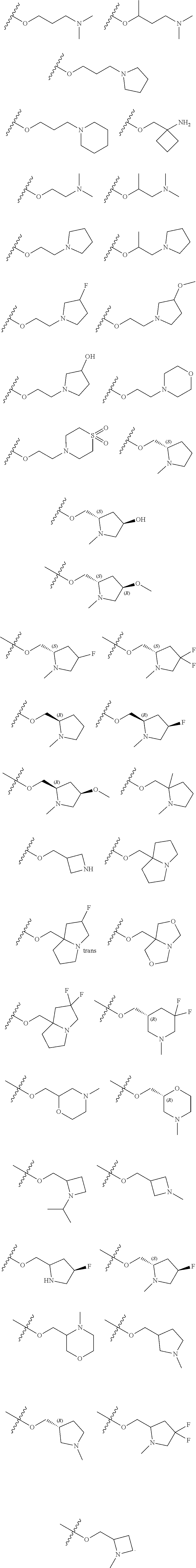

Embodiment 11. The compound of any one of Embodiments 1-6, or a pharmaceutically acceptable salt thereof, wherein R¹ is selected from:

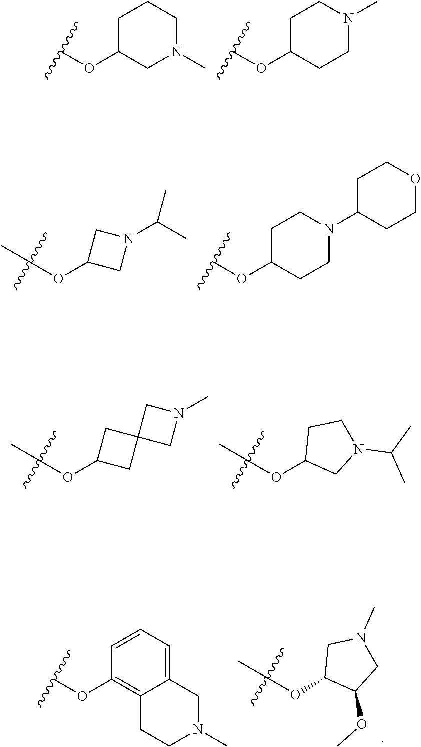

Embodiment 12. The compound of any one of Embodiments 1-6, or a pharmaceutically acceptable salt thereof, wherein R¹ is selected from:

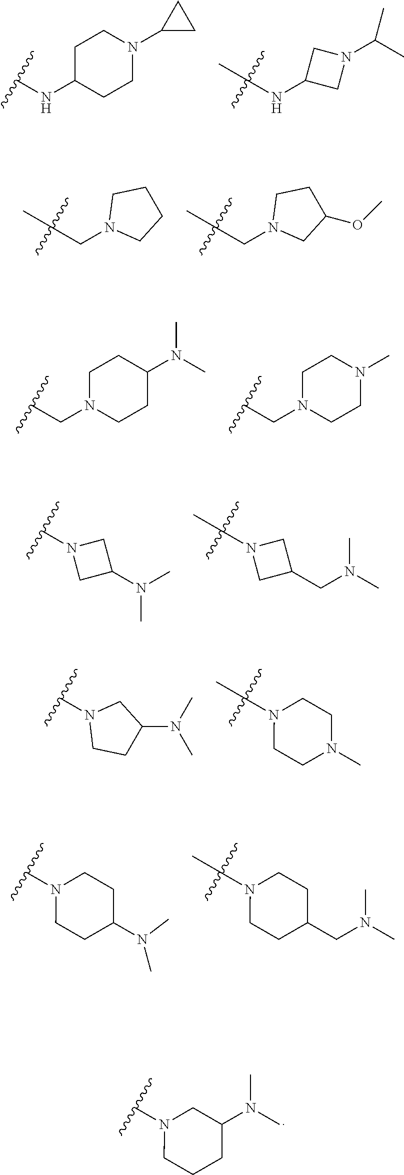

Embodiment 13. The compound of any one of Embodiments 1-6, or a pharmaceutically acceptable salt thereof, wherein R¹ is selected from:

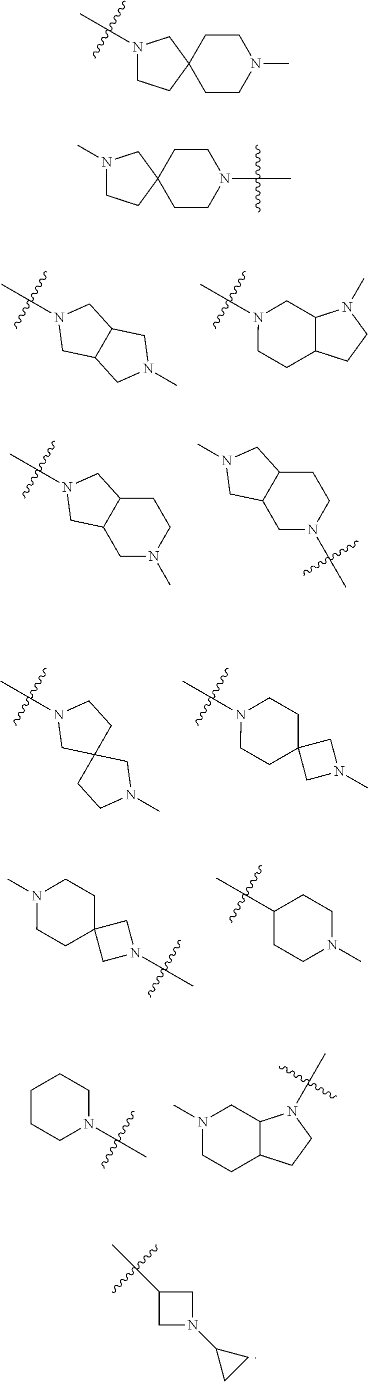

Embodiment 14. The compound of any one of Embodiments 1-6, or a pharmaceutically acceptable salt thereof, wherein R¹ is selected from:

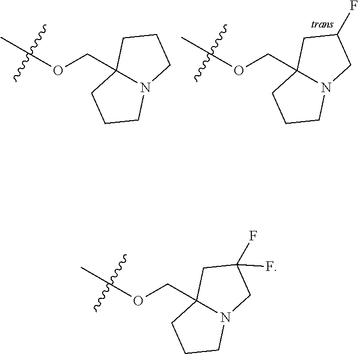

Embodiment 15. The compound of any one of Embodiments 1-6, or a pharmaceutically acceptable salt thereof, wherein R¹ is

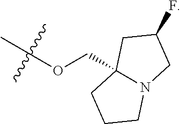 [SMILES: *OC[C@@]12CCCN1C[C@H](F)C2]

Embodiment 16. The compound of any one of Embodiments 1-6, or a pharmaceutically acceptable salt thereof, wherein R¹ is

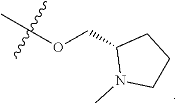 [SMILES: *OC[C@@H]1CCCN1C]

Embodiment 17. The compound of any one of Embodiments 1-6, or a pharmaceutically acceptable salt thereof, wherein R¹ is selected from:

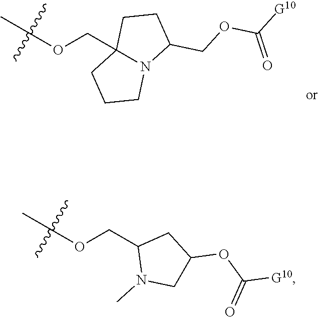

wherein G¹⁰ is amino, monoalkyl amino, dialkyl amino, or 4-10 membered heterocyclic ring, preferably, when G¹⁰ is a heterocyclic ring, the heterocyclic ring has a ring nitrogen bonded to the carbonyl group of the moiety to form a carbamate.
Embodiment 18. The compound of Embodiment 17, or a pharmaceutically acceptable salt thereof, wherein G¹⁰ is NH₂, NH(C₁₋₃₀ alkyl), N(CH₃)(C₁₋₃₀ alkyl),

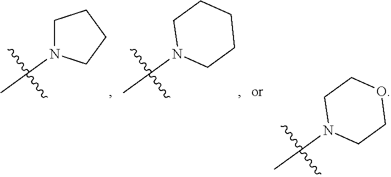 [SMILES: *N1CCCC1 ; *N1CCCCC1 ; *N1CCOCC1]

Embodiment 19. The compound of any one of Embodiments 1-6, or a pharmaceutically acceptable salt thereof, wherein R¹ is selected from:

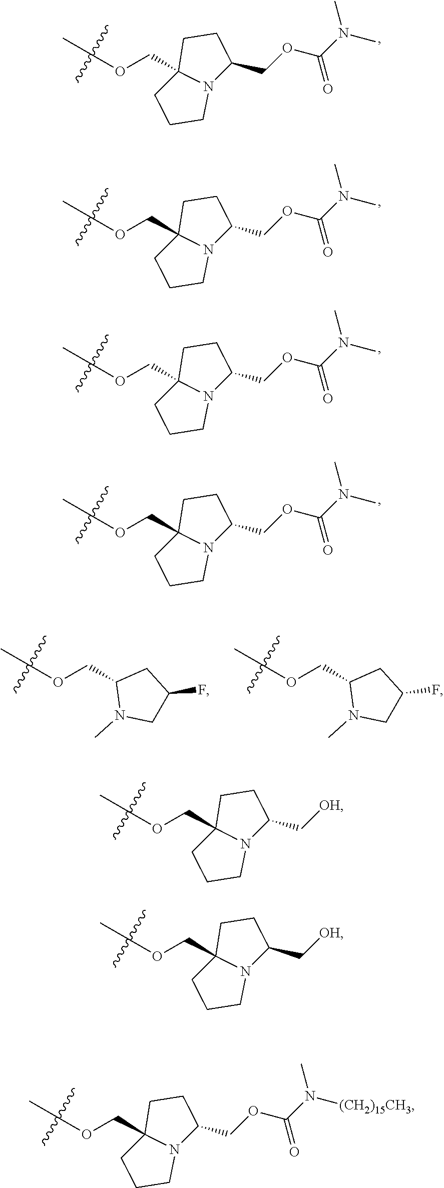

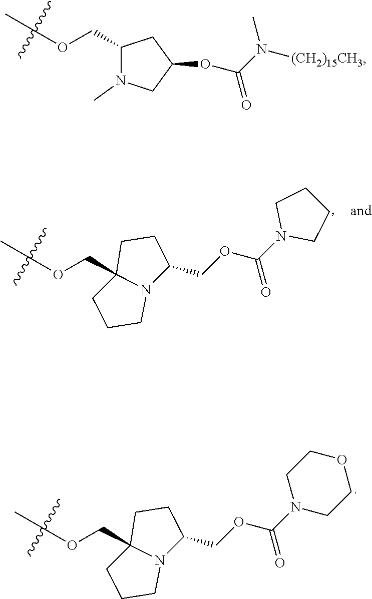

Embodiment 20. The compound of any one of Embodiments 1-6, or a pharmaceutically acceptable salt thereof, wherein R²⁰ is C₁₋₆ alkoxy optionally substituted with 1-3 F, such as methoxy, hydroxyl substituted C₁₋₆ alkoxy, such as hydroxyl ethoxy, alkoxy substituted C₁₋₆ alkoxy, such as methoxy ethoxy, or amino or alkyl amino substituted C₁₋₆ alkoxy, such as dimethylamino ethoxy.
Embodiment 21. The compound of any one of Embodiments 1-6, or a pharmaceutically acceptable salt thereof, wherein R¹ is NH₂, NH(C₁₋₆ alkyl), or N(C₁₋₆ alkyl)(C₁₋₆ alkyl).
Embodiment 22. The compound of any one of Embodiments 1-6, or a pharmaceutically acceptable salt thereof, wherein R¹ is methoxy,

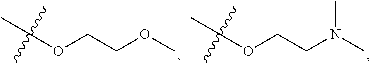

NH₂, NH(CH₃), or N (CH₃)₂.
Embodiment 22. The compound of any one of Embodiments 1-6, or a pharmaceutically acceptable salt thereof, wherein R¹ is —OR²⁰, wherein R²⁰ is a —C₁₋₆ alkylene-R¹⁰¹, wherein R¹⁰¹ is NR³²R³³ or an optionally substituted 4-10 membered heterocyclic ring, wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring; R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represent a monoalkyl or dialkyl amine; or R³² and R³³ are joined to form an optionally substituted heterocyclic or heteroaryl ring; and R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.
Embodiment 23. The compound of Embodiment 22, or a pharmaceutically acceptable salt thereof, wherein R²⁰ is —CH₂—R¹⁰¹, —CH₂—CH₂—R¹⁰¹, —CH₂—CH₂—CH₂—R¹⁰¹,

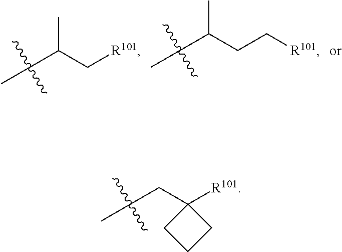

Embodiment 24. The compound of Embodiment 22, or a pharmaceutically acceptable salt thereof, wherein R²⁰ is

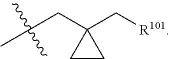

Embodiment 25. The compound of any one of Embodiments 22-24, or a pharmaceutically acceptable salt thereof, wherein R¹⁰¹ is NH₂, NH(C₁₋₃₀ alkyl), N(CH₃)(C₁₋₃₀ alkyl),

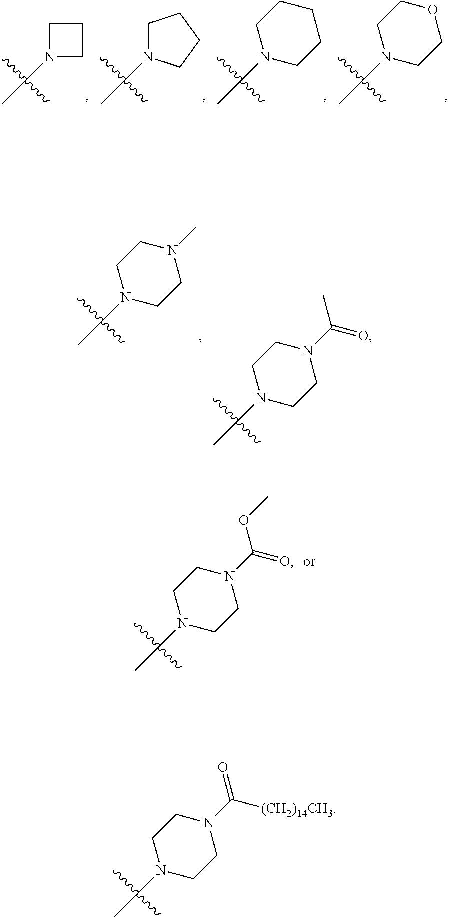

Embodiment 26. The compound of any one of Embodiments 1-6, or a pharmaceutically acceptable salt thereof, wherein R¹ is

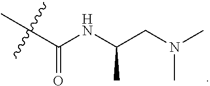 [SMILES: *C(=O)N[C@H](C)CN(C)C]

Embodiment 27. The compound of any one of Embodiments 1-26, or a pharmaceutically acceptable salt thereof, wherein R² is selected from:

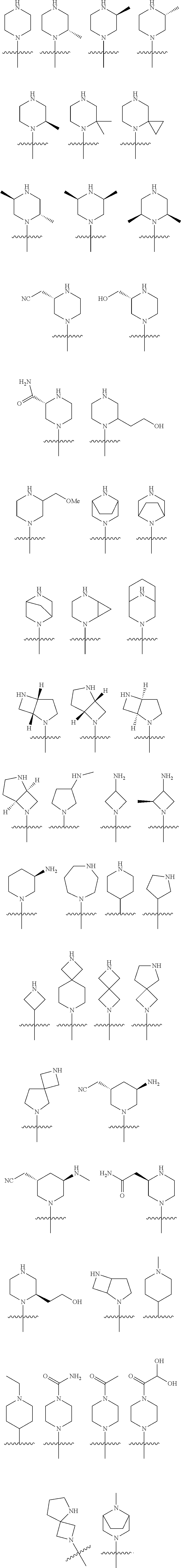

Embodiment 28. The compound of any one of Embodiments 1-26, or a pharmaceutically acceptable salt thereof, wherein R² is selected from:

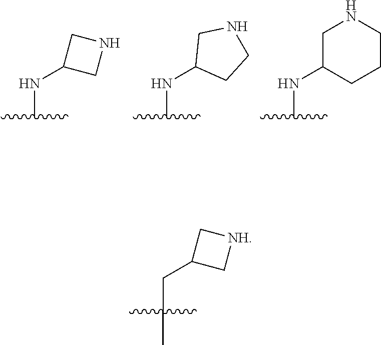 [SMILES: *NC1CNC1 ; *NC1CCNC1 ; *NC1CCCNC1 ; *CC1CNC1]

Embodiment 29. The compound of any one of Embodiments 1-26, or a pharmaceutically acceptable salt thereof, wherein R² is selected from:

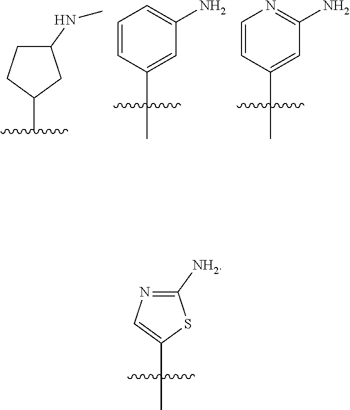

Embodiment 30. The compound of any one of Embodiments 1-26, or a pharmaceutically acceptable salt thereof, wherein R² is:

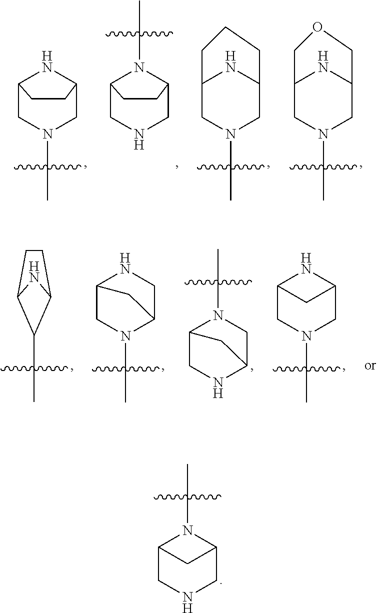

Embodiment 31. The compound of any one of Embodiments 1-26, or a pharmaceutically acceptable salt thereof, wherein R² is

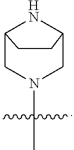 [SMILES: *N1CC2CCC(C1)N2]

Embodiment 32. The compound of any one of Embodiments 1-26, or a pharmaceutically acceptable salt thereof, wherein R² is

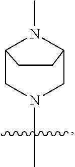 [SMILES: *N1CC2CCC(C1)N2C]

Embodiment 33. The compound of any one of Embodiments 1-26, or a pharmaceutically acceptable salt thereof, wherein R² is

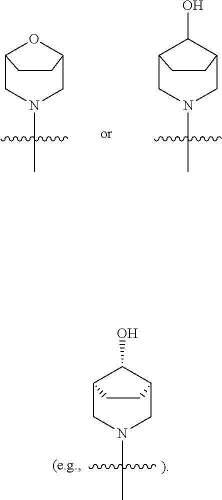 [SMILES: *N1CC2CCC(C1)O2 ; *N1CC2CCC(C1)C2O ; *N1C[C@H]2CC[C@@H](C1)[C@@H]2O]

Embodiment 34. The compound of any one of Embodiments 1-26, or a pharmaceutically acceptable salt thereof, wherein R² is

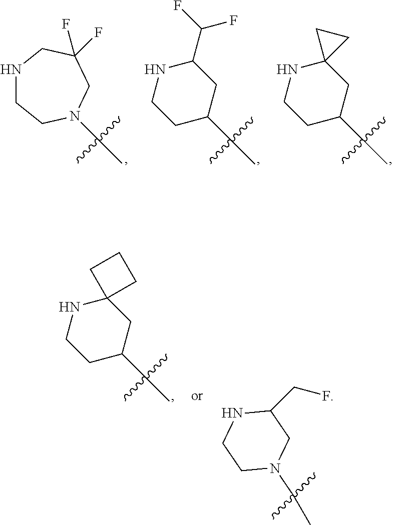 [SMILES: *N1CCNCC(F)(F)C1 ; *C1CCNC(C(F)F)C1 ; *C1CCNC2(CC2)C1 ; *C1CCNC2(CCC2)C1 ; *N1CCNC(CF)C1]

Embodiment 35. The compound of any one of Embodiments 1-34, or a pharmaceutically acceptable salt thereof, wherein R³ is selected from:

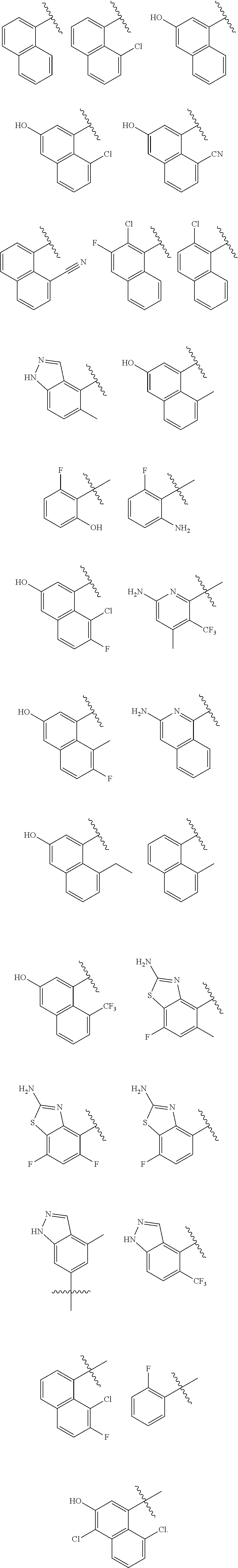

Embodiment 36. The compound of any one of Embodiments 1-34, or a pharmaceutically acceptable salt thereof, wherein R³ is selected from:

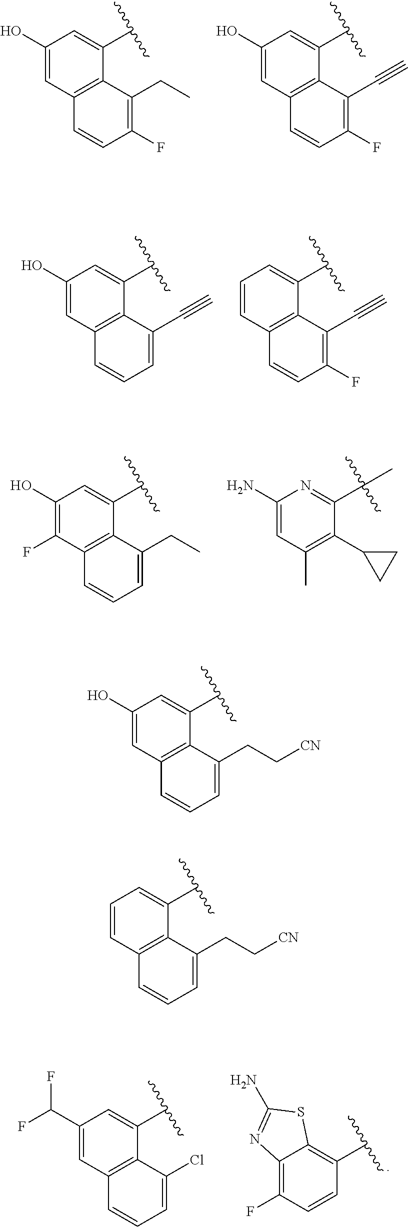

Embodiment 37. The compound of any one of Embodiments 1-34, or a pharmaceutically acceptable salt thereof, wherein R³ is

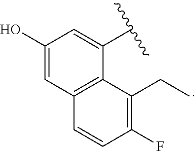 [SMILES: *c1cc(O)cc2ccc(F)c(CC)c12]

Embodiment 38. The compound of any one of Embodiments 1-34, or a pharmaceutically acceptable salt thereof, wherein R³ is

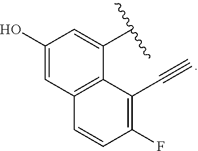 [SMILES: *c1cc(O)cc2ccc(F)c(C#C)c12]

Embodiment 39. The compound of any one of Embodiments 1-34, or a pharmaceutically acceptable salt thereof, wherein R³ is

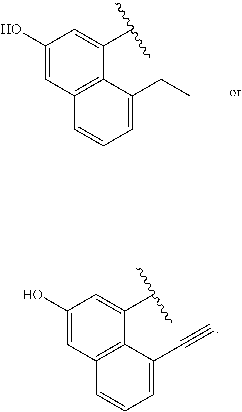 [SMILES: *c1cc(O)cc2cccc(CC)c12 ; *c1cc(O)cc2cccc(C#C)c12]

Embodiment 40. The compound of any one of Embodiments 1-34, or a pharmaceutically acceptable salt thereof, wherein R³ is

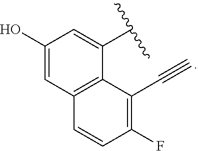 [SMILES: *c1cc(O)cc2ccc(F)c(C#C)c12]

Embodiment 41. The compound of any one of Embodiments 1-34, or a pharmaceutically acceptable salt thereof, wherein R³ is

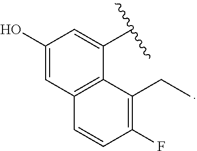 [SMILES: *c1cc(O)cc2ccc(F)c(CC)c12]

Embodiment 42. The compound of any one of Embodiments 1-34, or a pharmaceutically acceptable salt thereof, wherein R³ is

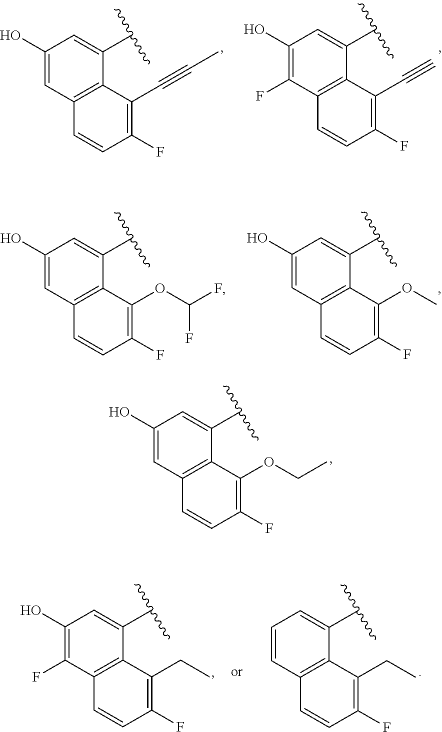

Embodiment 43. The compound of any one of Embodiments 1-34, or a pharmaceutically acceptable salt thereof, wherein R³ is

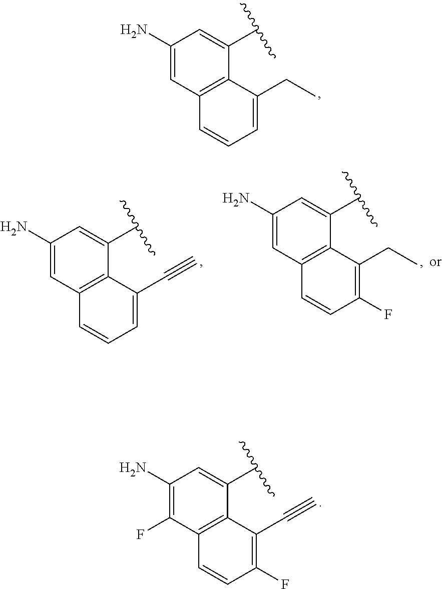 [SMILES: *c1cc(N)cc2cccc(CC)c12 ; *c1cc(N)cc2cccc(C#C)c12 ; *c1cc(N)cc2ccc(F)c(CC)c12 ; *c1cc(N)c(F)c2ccc(F)c(C#C)c12]

Embodiment 44. The compound of any one of Embodiments 1-34, or a pharmaceutically acceptable salt thereof, wherein R³ is

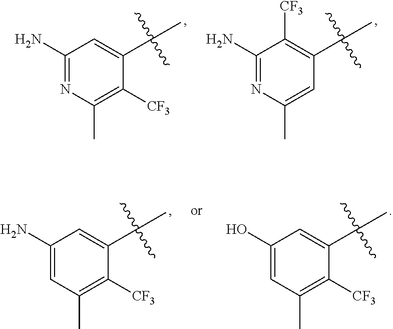 [SMILES: *c1cc(N)nc(C)c1C(F)(F)F ; *c1cc(C)nc(N)c1C(F)(F)F ; *c1cc(N)cc(C)c1C(F)(F)F ; *c1cc(O)cc(C)c1C(F)(F)F]

Embodiment 45. A compound according to Formula I-11:

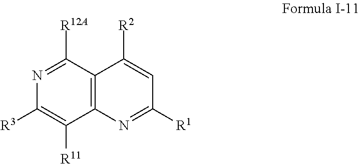

or a pharmaceutically acceptable salt thereof,
wherein the variables R¹, R², R³, R¹¹, and R^{12A} include any of those described herein in connection with Formula I-11 in any combination.
Embodiment 46. The compound of Embodiment 45, or a pharmaceutically acceptable salt thereof, wherein R¹¹ is F.
Embodiment 47. The compound of Embodiment 44 or 45, or a pharmaceutically acceptable salt thereof, wherein R^{12A} is any of those defined in Embodiments 3-5.
Embodiment 48. The compound of any of Embodiments 44-47, or a pharmaceutically acceptable salt thereof, wherein R¹ is any of those defined in Embodiments 7-26.
Embodiment 49. The compound of any of Embodiments 44-48, or a pharmaceutically acceptable salt thereof, wherein R² is any of those defined in Embodiments 27-34.
Embodiment 50. The compound of any of Embodiments 44-49, or a pharmaceutically acceptable salt thereof, wherein R³ is any of those defined in Embodiments 35-44.
Embodiment 51. A compound according to one of the following formulae:

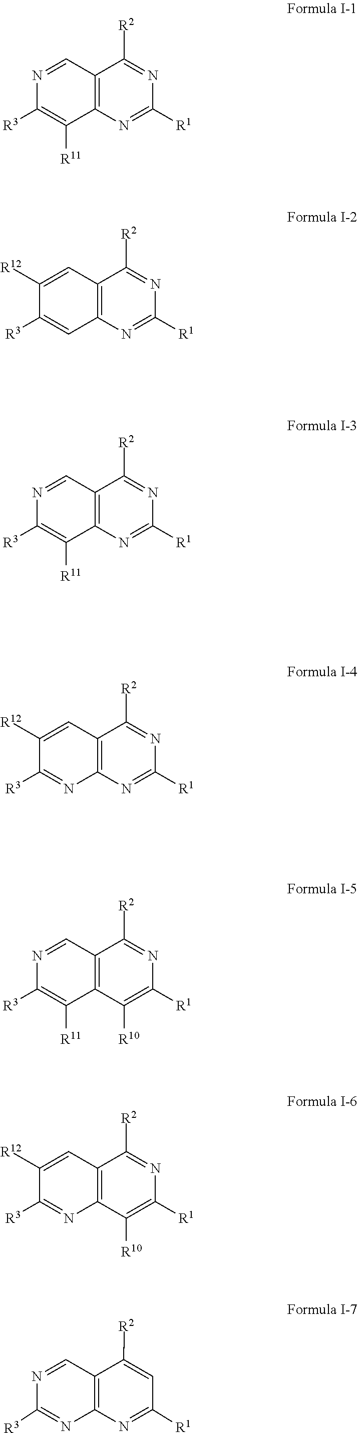

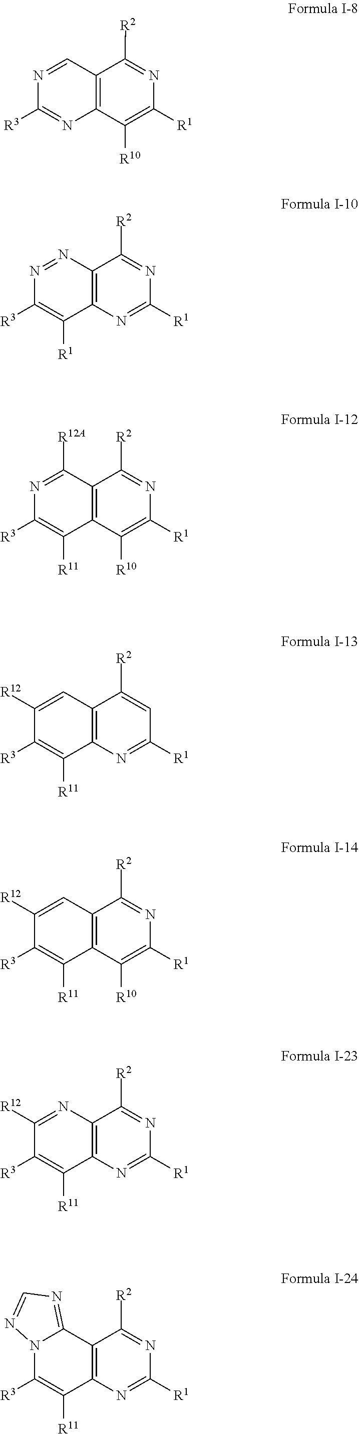

or a pharmaceutically acceptable salt thereof,
wherein the variables R¹, R², R³, R¹¹, R¹² and R^{12A} include any of those described herein in connection with the respective formula in any combination.
Embodiment 52. The compound of Embodiment 51, or a pharmaceutically acceptable salt thereof, wherein when present, R¹¹ is F.
Embodiment 53. The compound of Embodiment 51 or 52, or a pharmaceutically acceptable salt thereof, wherein when present, R^{12A} is any of those defined in Embodiments 3-5.
Embodiment 54. The compound of Embodiment 51 or 52, or a pharmaceutically acceptable salt thereof, wherein when present, R¹² is F, Cl, —CN, —OH, methoxy, ethoxy, —O—CH₂-cyclopropyl, —C(O)NHMe, CF₃, methyl, ethyl, isopropyl, or cyclopropyl.
Embodiment 55. The compound of any of Embodiments 51-54, or a pharmaceutically acceptable salt thereof, wherein R¹ is any of those defined in Embodiments 7-26.
Embodiment 56. The compound of any of Embodiments 51-55, or a pharmaceutically acceptable salt thereof, wherein R² is any of those defined in Embodiments 27-34.
Embodiment 57. The compound of any of Embodiments 51-56, or a pharmaceutically acceptable salt thereof, wherein R³ is any of those defined in Embodiments 35-44.

In some embodiments, the present disclosure provides a compound of Formula A, or a pharmaceutically acceptable salt thereof:

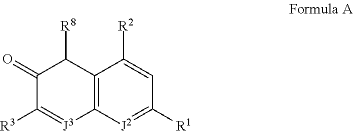

wherein:
R⁸ is hydrogen, optionally substituted C₁₋₆ alkyl (e.g., methyl), or optionally substituted C₃₋₁₀ cycloalkyl, and
R¹, R², R³, J¹, J², and J³ include any of those described herein in connection with Formula I (e.g., its sub-formulae) in any combination. For the avoidance of doubt, when a variable of Formula A is said to have or include the definition of any of those described herein in connection with Formula I, it should be understood that the variable can have or include the definition of the variable having the same identifier, e.g., R² in Formula A can have or include the definition of R² described herein in connection with Formula A. Other similar expressions herein should be understood similarly. Suitable J¹, J², and J³ definitions for Formula A include any of those described herein in connection with Formula I (or its sub-formulae) in any combination. For example, in some embodiments, when present, R¹¹ in Formula A is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, J¹ is CH or N. In some embodiments, J² is CH or N.

The compound of Formula A (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula A (including any of the applicable sub-formulae as described herein) can exist as a mixture of atropisomers in any ratio, including about 1:1. In some embodiments, when applicable, the compound of Formula A (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer.

In some embodiments, the compound of Formula A can be characterized as having Formula A-1:

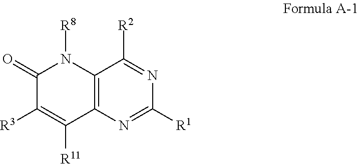

R¹, R², R³, R¹¹, and R⁸ include any of those described herein in connection with Formula I (e.g., its sub-formulae) in any combination. For example, in some embodiments, R⁸ is hydrogen. In some embodiments, R⁸ is an optionally substituted C₁₋₆ alkyl (e.g., methyl), suitable substituents include any of those described herein for C₁₋₆ alkyl.

In some embodiments, R¹ in Formula A (e.g., Formula A-1) is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. For example, in some embodiments, R¹ in Formula A (e.g., Formula A-1) is

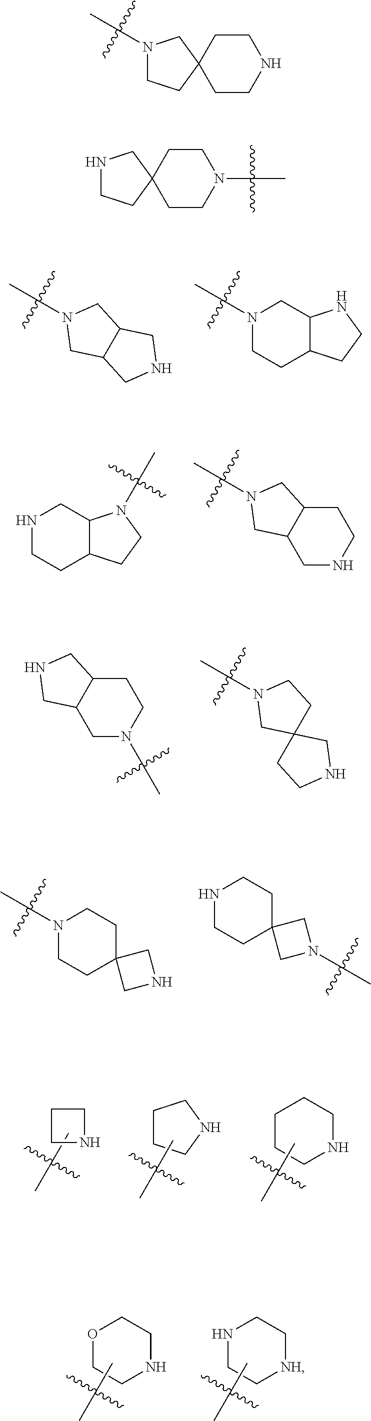

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some embodiments, R¹ in Formula A (e.g., Formula A-1) is —OR²⁰, wherein R²⁰ is a —C₁₋₆ alkylene-R¹⁰¹, wherein R¹⁰¹ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring; R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represent a monoalkyl or dialkyl amine; or R³² and R³³ are joined to form an optionally substituted heterocyclic or heteroaryl ring; and
   R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.
   Suitable “—C₁₋₆ alkylene-” and R¹⁰¹ include any of those described herein having the same respective identifiers in connection with R²⁰ defined in Formula I (including subformulae). For example, in some embodiments, the “—C₁₋₆ alkylene-” is —CH₂—, —CH₂—CH₂—, —CH₂—CH₂—CH₂—,

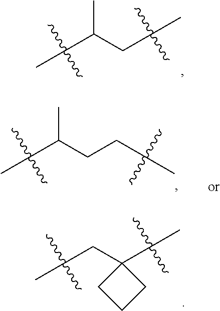

In some embodiments, R¹⁰¹ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. In some embodiments, R¹⁰¹ is a monocyclic ring selected from the following:

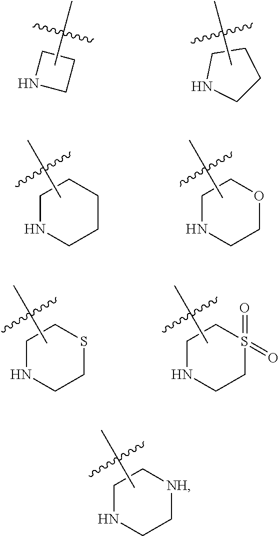

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, R¹⁰¹ is a bicyclic ring selected from the following:

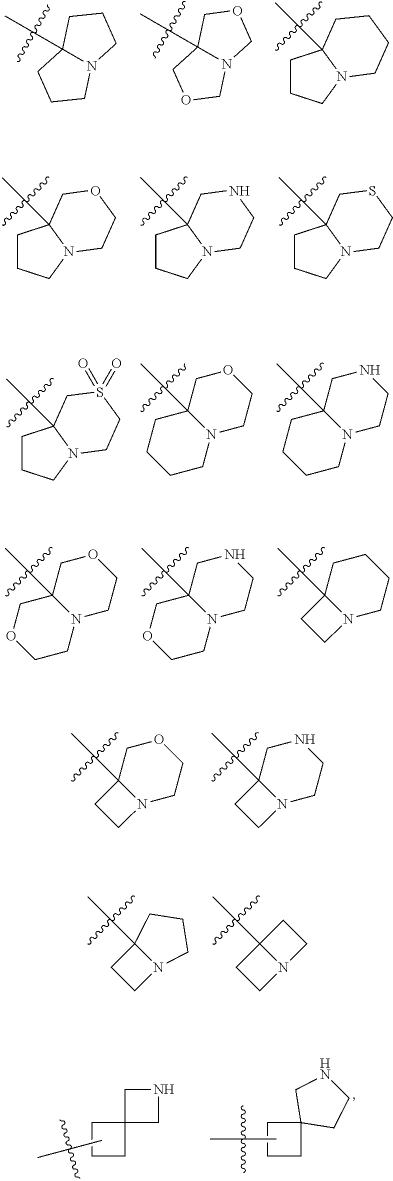

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹ in Formula A (e.g., Formula A-1) is selected from:

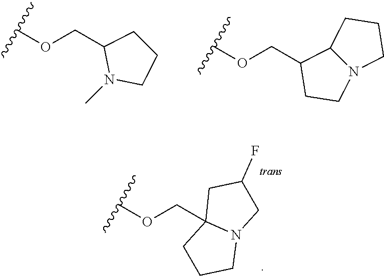

Typically, R² in Formula A does not contain a Michael acceptor, such as an alpha-beta unsaturated carbonyl structural moiety. In some embodiments, R² in Formula A can be represented by -(L²)ₘ₂-R¹⁰², wherein m2 is 0-3, typically 0 or 1, and when m2 is not 0, for example, m2 is 1, L² at each occurrence is independently CH₂, O, NH, or NCH₃, R¹⁰² is an optionally substituted 4-10 membered heterocyclic ring or a heteroaryl ring, e.g., those heterocyclic or heteroaryl rings having one or two ring nitrogen atoms. In some embodiments, m2 is 0. In some embodiments, m2 is 1. In some embodiments, R² in Formula A (e.g., Formula A-1) is -(L²)ₘ₂-R¹⁰², wherein
m2 is 0 or 1, and when m2 is 1, L² is CH₂, O, NH, or NCH₃,
R¹⁰² is an optionally substituted 4-10 membered heterocyclic or heteroaryl ring having one or two ring nitrogen atoms.

Suitable R¹⁰² includes any of those described herein in connection with Formula I (e.g., any of its sub-formulae). In some embodiments, R¹⁰² is an optionally substituted 4-10 membered heterocyclic ring having one or two ring nitrogen atoms. In some embodiments, R¹⁰² or R² in Formula A (e.g., Formula A-1) is selected from:

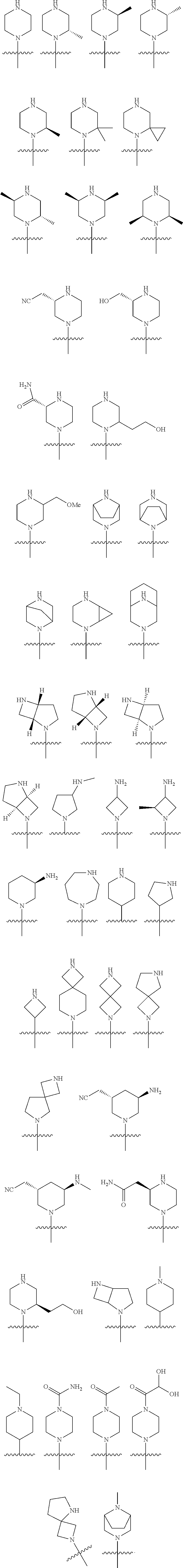

or selected from

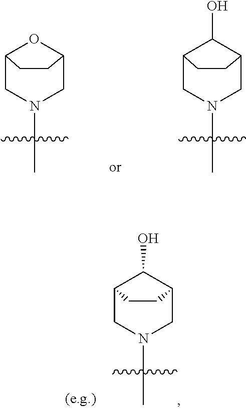 [SMILES: *N1CC2CCC(C1)O2 ; *N1CC2CCC(C1)C2O ; *N1C[C@H]2CC[C@@H](C1)[C@@H]2O]

or selected from

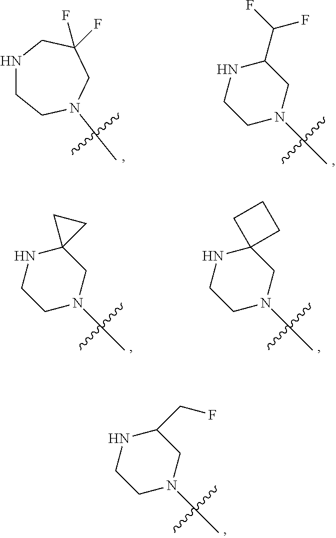 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

Suitable R for Formula A (e.g., Formula A-1) include any of those described herein in connection with Formula I and those exemplified herein in the specific examples. For example, in some embodiments, R³ in Formula A (e.g., Formula A-1) is a phenyl, pyridyl, naphthyl, or bicyclic heteroaryl (e.g., benzothiazolyl, indazolyl, or isoquinolinyl) each of which is optionally substituted, for example, with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, R³ in Formula A (e.g., Formula A-1) is selected from

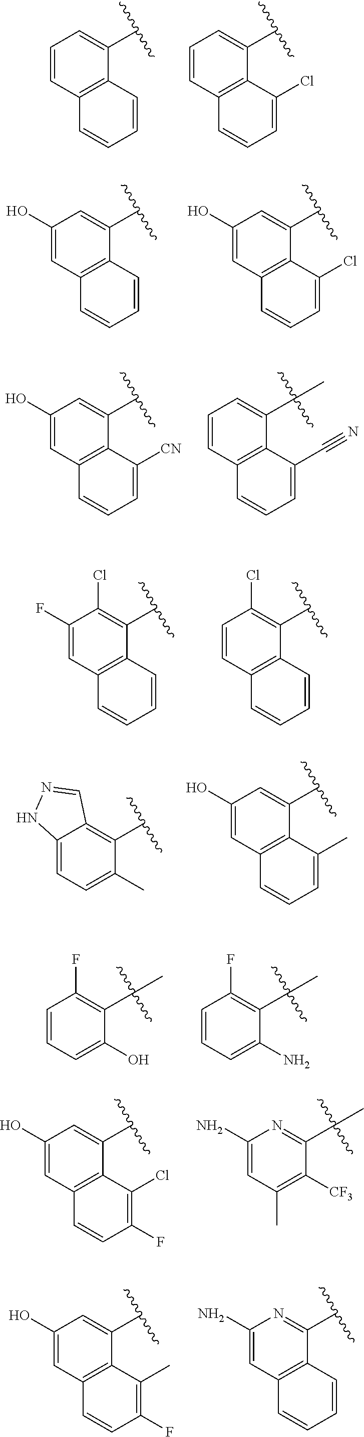

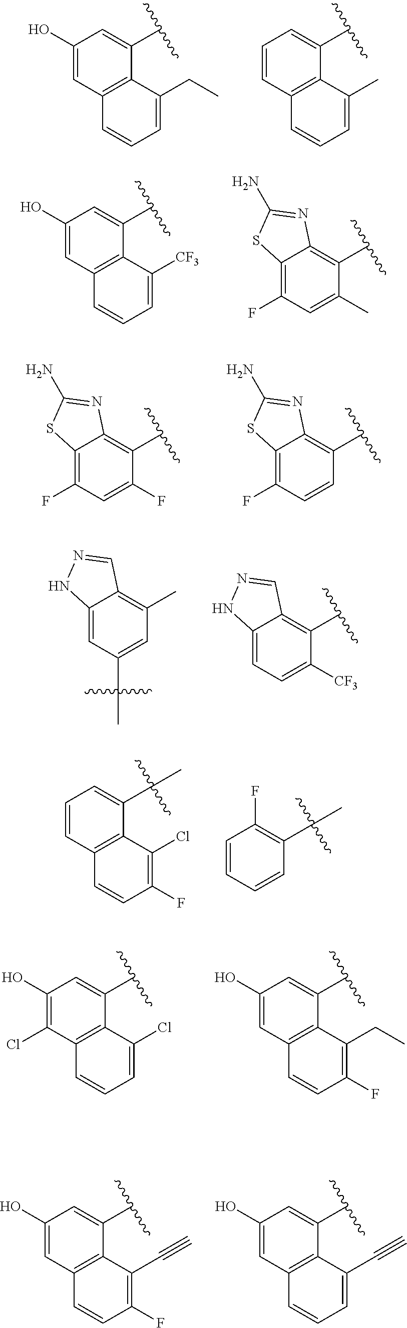

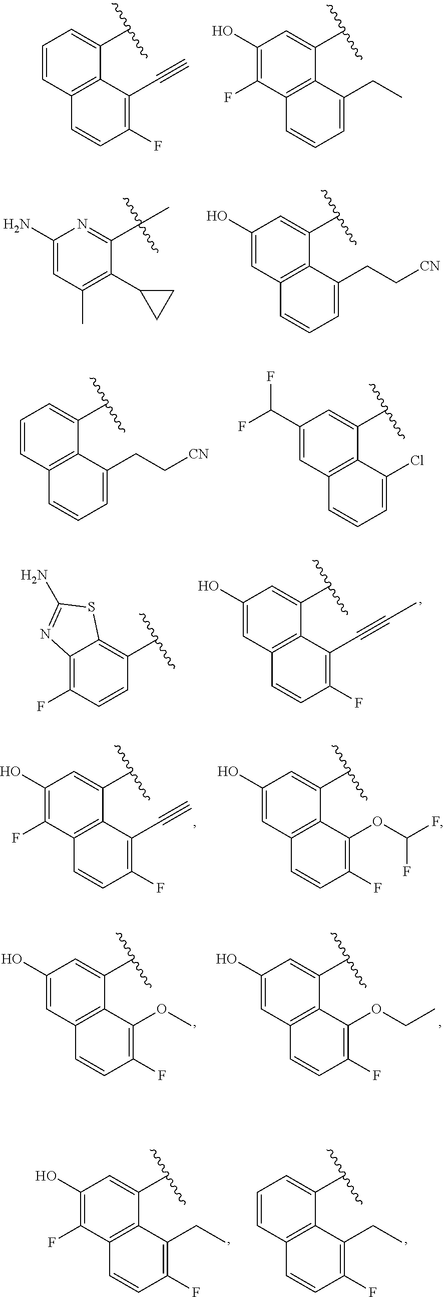

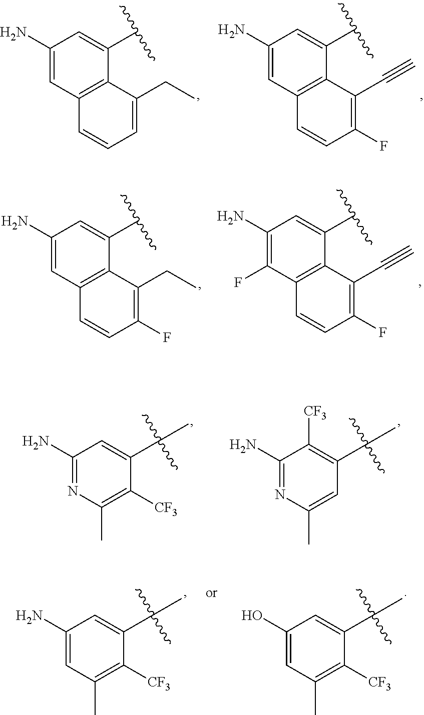

In some embodiments, the present disclosure provides a compound of Formula II, or a pharmaceutically acceptable salt thereof:

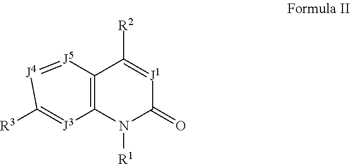

wherein:
J¹ is CR⁹ or N;
J³ is CR¹¹ or N;
J⁴ is CR¹² or N;
J⁵ is CR^{12A} or N;
or J⁴ and J⁵ are joined to form an optionally substituted phenyl or optionally substituted 5, or 6-membered heteroaryl, provided that in such cases, the bond between J⁴ and J⁵ can be a single bond, for example, when J⁴ and J⁵ are joined to form a triazole ring;
R¹ is hydrogen, an optionally substituted C₁₋₆ alkyl, an optionally substituted carbocyclic ring, an optionally substituted aryl, -(L¹)ₘ₁-OR²⁰, (L¹)ₘ₁-NR³⁰R³¹, —C(O)—NR³R³, or an optionally substituted heterocyclic or heteroaryl ring;
R² is a ring or ring-chain structure, e.g., those having a basic functional group with a pKa of the conjugated acid of about 6 or higher, or an acylated derivative thereof (i.e., the basic functional group, such as a basic NH, is bonded with an acyl group);
R³ is an optionally substituted aryl or an optionally substituted heteroaryl,
R⁹ is hydrogen, halogen, cyano, optionally substituted C₁₋₄ alkyl (e.g., methyl, ethyl, CF₃, etc.), optionally substituted C₂₋₄ alkenyl, optionally substituted C₂₋₄ alkynyl, optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹, R¹² and R^{12A} at each occurrence is independently hydrogen, F, Cl, Br, I, CN, —OH, —C(O)NH₂, —NH₂, —NH(C₁₋₆ alkyl), —N(C₁₋₆ alkyl)(C₁₋₆ alkyl), —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), optionally substituted C₂₋₄ alkenyl, optionally substituted C₂₋₄ alkynyl, optionally substituted C₃₋₆ cycloalkyl (e.g., cyclopropyl, cyclobutyl), optionally substituted C₁₋₄ alkoxy (e.g., methoxy, ethoxy, —O—CH₂-cyclopropyl), optionally substituted C₃₋₆ cycloalkoxy (e.g., cyclopropoxy, or cyclobutoxy), optionally substituted 4-7 membered heterocyclic, or optionally substituted 4-7 membered heterocycloalkoxy; or R¹² and R^{12A} are joined to form a 5-7 membered ring structure; and
   wherein:
   m1 is 0 or 1, and when m1 is 1, L¹ is an optionally substituted alkylene, an optionally substituted carbocyclylene, an optionally substituted heterocyclylene;
   R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
   R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring; or one of R³⁰ and R³¹ together with a suitable atom of L¹ and any intervening atoms form an optionally substituted heterocyclic or heteroaryl ring.

The compound of Formula II (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula II (including any of the applicable sub-formulae as described herein) can exist as a mixture of atropisomers in any ratio, including about 1:1. In some embodiments, when applicable, the compound of Formula II (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer.

Suitable R² and R³ groups for Formula II include any of those described herein in connection with Formula I (e.g., its sub-formulae) in any combination. For the avoidance of doubt, when a variable of Formula II is said to have or include the definition of any of those described herein in connection with Formula I, it should be understood that the variable can have or include the definition of the variable having the same identifier, e.g., R² in Formula II can have or include the definition of R² described herein in connection with Formula I. Other similar expressions should be understood similarly. Suitable J¹, J³, J⁴, and J⁵ definitions for Formula II also include any of those described herein in connection with Formula I (or its sub-formulae) in any combination. For example, in some embodiments, when present, R¹¹ in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, when present, R¹² in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, when present, R^{12A} in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl, such as hydrogen, chloro, or methyl. In some embodiments, when present, R^{12A} in Formula II is hydrogen, methyl, Cl, or methoxy. In some embodiments, J⁴ and J⁵ are joined to form an optionally substituted 5, or 6-membered heteroaryl, provided that in such cases, the bond between J⁴ and J⁵ can be a single bond. For example, in some embodiments, J⁴ and J⁵ are joined to form a triazole ring.

In some embodiments, the compound of Formula II can have one of the following subformulae:

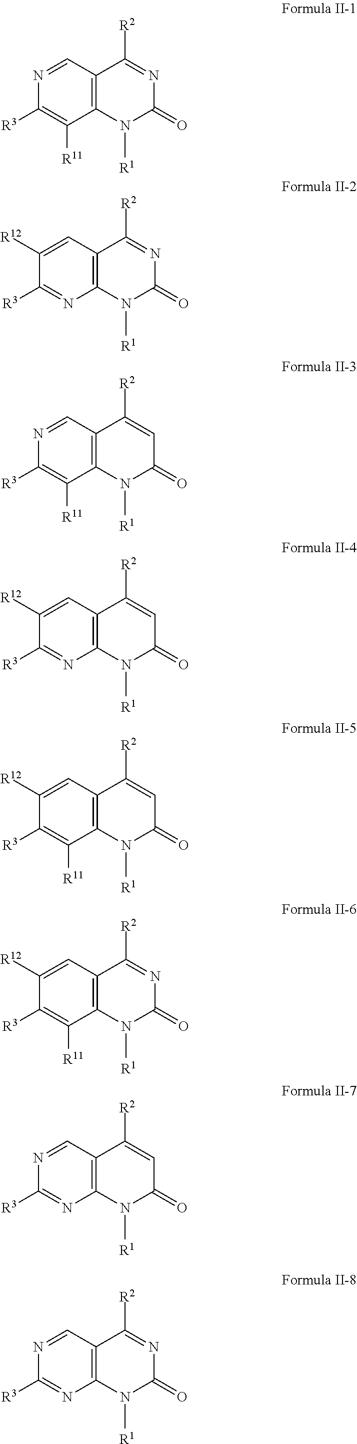

wherein R¹, R², R³, R¹¹, and R¹² include any of those defined herein in any combinations.

In some embodiments, R¹ in Formula II (e.g., Formula II-1, II-2, II-3, II-4, II-5, II-6, II-7, or II-8) is a substituted alkyl having the formula: —C₁₋₆ alkylene-R¹⁰¹, wherein R¹⁰¹ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring;
R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represent a monoalkyl or dialkyl amine; or R³² and R³³ are joined to form an optionally substituted heterocyclic or heteroaryl ring; and R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.
Suitable “—C₁₋₆ alkylene-” and R¹⁰¹ include any of those described herein having the same respective identifiers in connection with R²⁰ defined in Formula I (e.g., any of its sub-formulae). For example, in some embodiments, the “—C₁₋₆ alkylene-” is —CH₂—, —CH₂—CH₂—, —CH₂—CH₂—CH₂—,

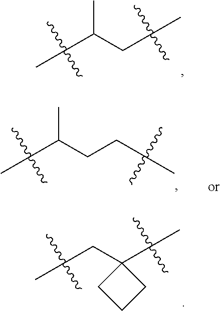

In some embodiments, R¹⁰¹ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. In some embodiments, R¹⁰¹ is a monocyclic ring selected from the following:

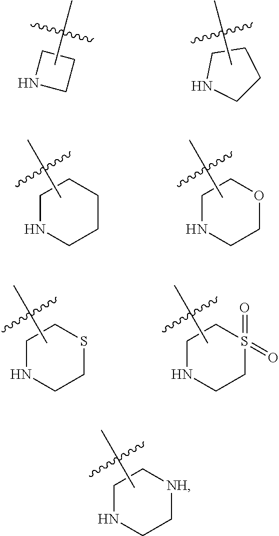

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, R¹⁰¹ is a bicyclic ring selected from the following:

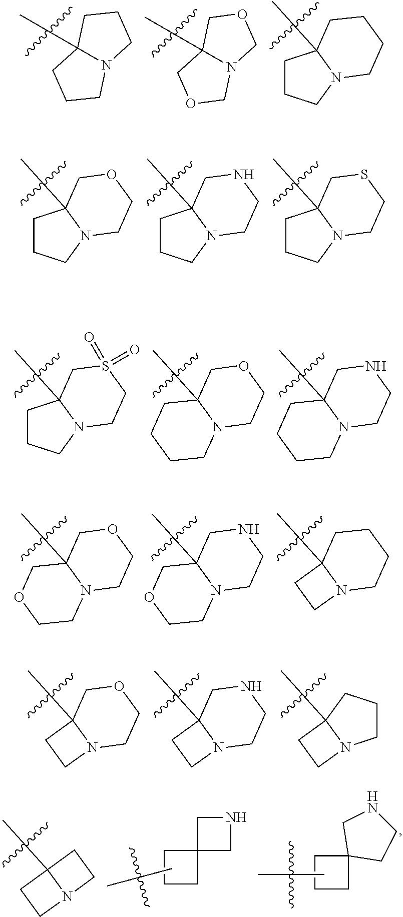

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹ in Formula II (e.g., Formula II-1, II-2, II-3, II-4, II-5, II-6, II-7, or II-8) is —C₁₋₆ alkylene-NR³⁰R³¹, wherein R³⁰ and R³¹ are independently hydrogen, an optionally substituted C₁₋₆ alkyl, or an optionally substituted heterocyclic ring; or R³⁰ and R³¹ together with the N they are both attached to are joined to form an optionally substituted heterocyclic ring having one or two ring heteroatoms, or one of R³⁰ and R³¹ together with a CH₂ unit of the C₁₋₆ alkylene and any intervening atoms form an optionally substituted heterocyclic or heteroaryl ring having one or two ring heteroatoms.

In some embodiments, R¹ in Formula II (e.g., Formula II-1, II-2, II-3, II-4, II-5, II-6, II-7, or II-8) is —C₁₋₆ alkylene-NR³⁰R³,
wherein R³⁰ together with a CH₂ unit of the C₁₋₆ alkylene and any intervening atoms form a ring selected from (R³¹ is shown):

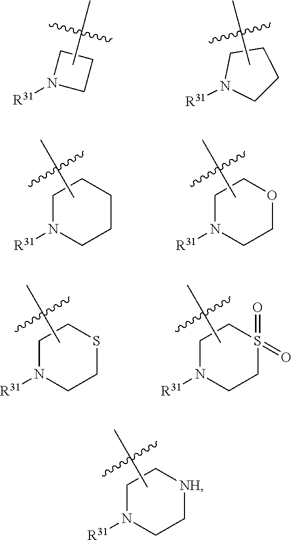

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, R³¹ is —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₚ-cyclopropyl, —(CH₂)ₚ-cyclobutyl, or —(CH₂)ₚ-(4-6 membered heterocyclic ring having 1 or 2 ring heteroatoms independently selected from O, N, and S), wherein x is 1, 2, or 3, preferably, 2 or 3, and p is 0, 1, 2, or 3.

In some embodiments, R¹ in Formula II (e.g., Formula II-1, II-2, II-3, II-4, II-5, II-6, II-7, or II-8) is —C₁₋₆ alkylene-NR³⁰R³¹,
wherein R³⁰ and R³¹ together with the N they are both attached to are joined to form a ring selected from

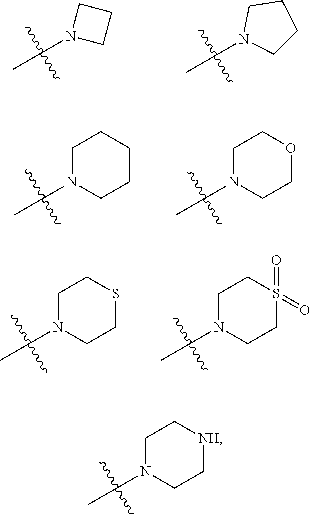

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some particular embodiments, R¹ in Formula II (e.g., Formula II-1, II-2, II-3, II-4, II-5, II-6, II-7, or II-8) is selected from

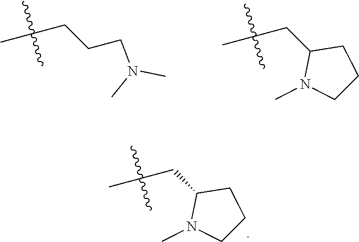

Suitable R² for Formula II (e.g., Formula II-1, II-2, II-3, II-4, II-5, II-6, II-7, or II-8) include any of those described herein in connection with Formula I and those exemplified herein in the specific examples. Typically, R² in Formula II does not contain a Michael acceptor, such as an alpha-beta unsaturated carbonyl structural moiety. For example, in some embodiments, R² for Formula II (e.g., Formula II-1, II-2, II-3, II-4, II-5, II-6, II-7, or II-8) is selected from

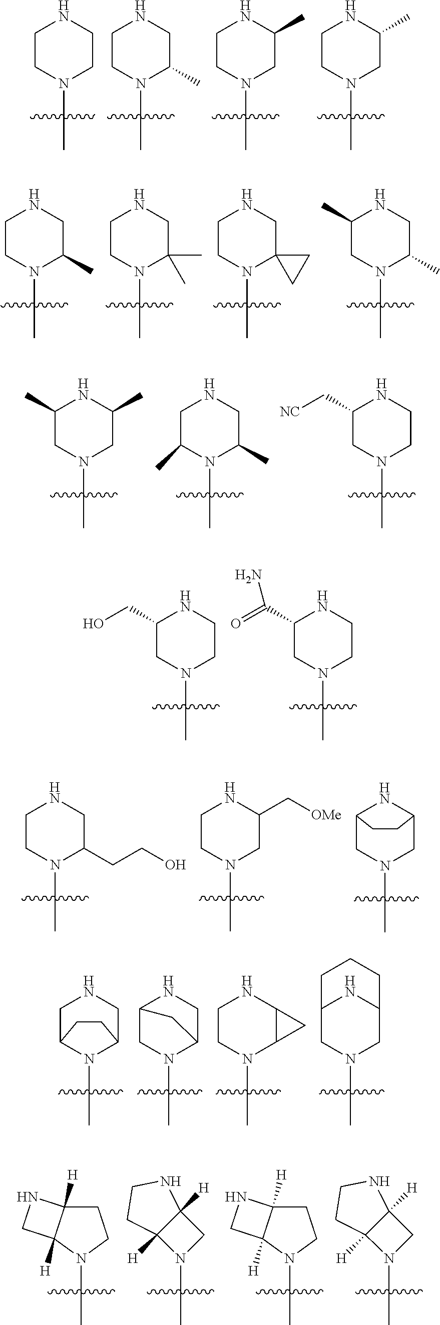

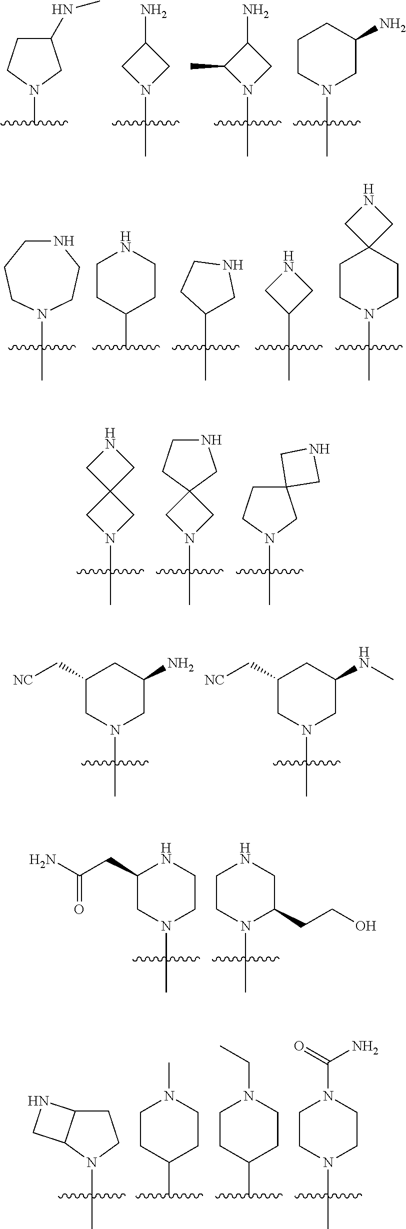

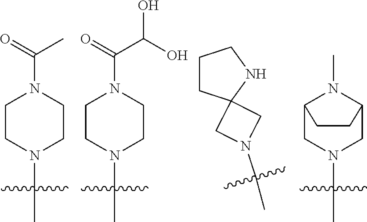

or selected from

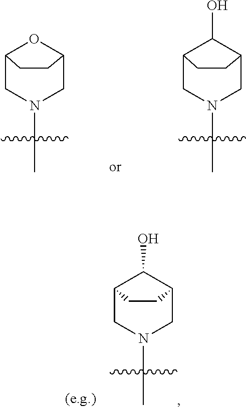 [SMILES: *N1CC2CCC(C1)O2 ; *N1CC2CCC(C1)C2O ; *N1C[C@H]2CC[C@@H](C1)[C@@H]2O]

or selected from

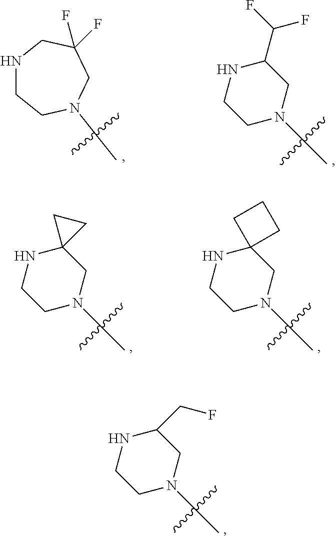 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

Suitable R³ for Formula II (e.g., Formula II-1, II-2, II-3, II-4, II-5, II-6, II-7, or II-8) include any of those described herein in connection with Formula I and those exemplified herein in the specific examples. For example, in some embodiments, R³ in Formula II (e.g., Formula II-1, II-2, II-3, II-4, II-5, II-6, II-7, or II-8) is a phenyl, pyridyl, naphthyl, or bicyclic heteroaryl (e.g., benzothiazolyl, indazolyl, or isoquinolinyl) each of which is optionally substituted with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, R³ in Formula II (e.g., Formula II-1, II-2, II-3, II-4, II-5, II-6, II-7, or II-8) can be a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, R³ in Formula II (e.g., Formula II-1, II-2, II-3, II-4, II-5, II-6, II-7, or II-8) is selected from

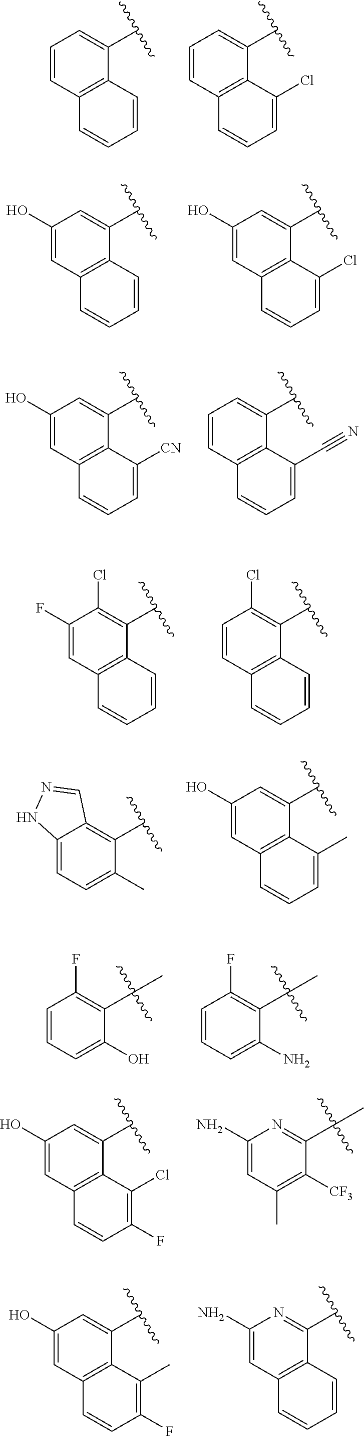

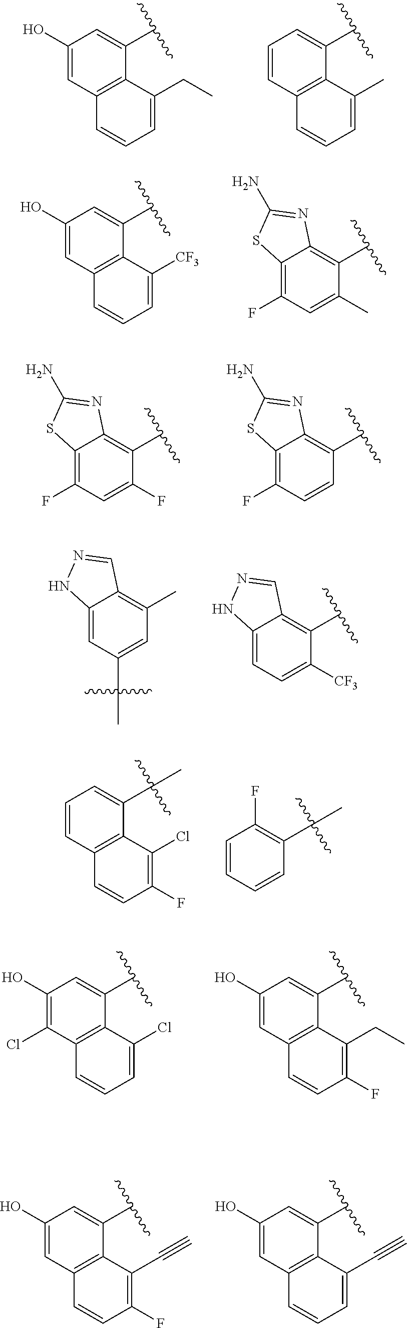

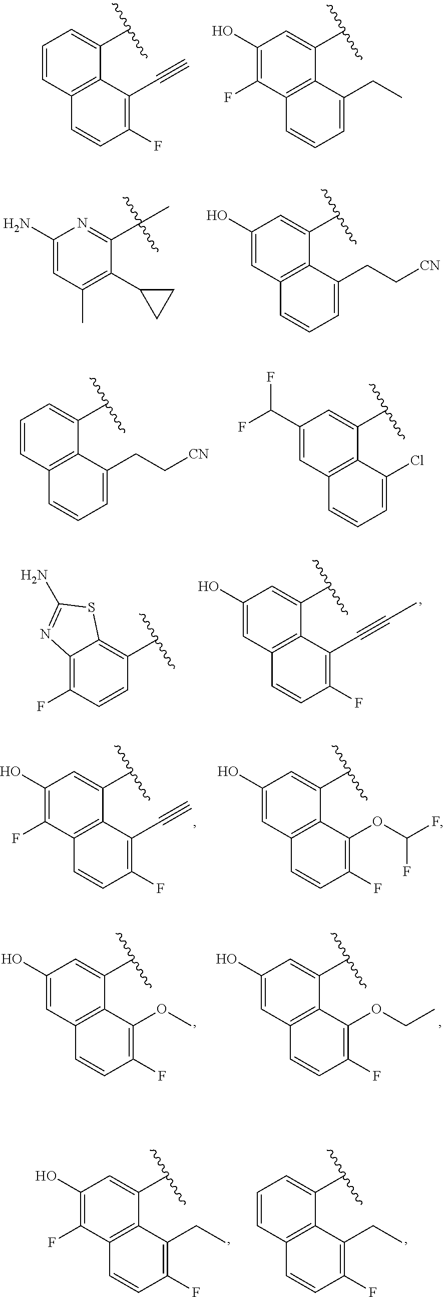

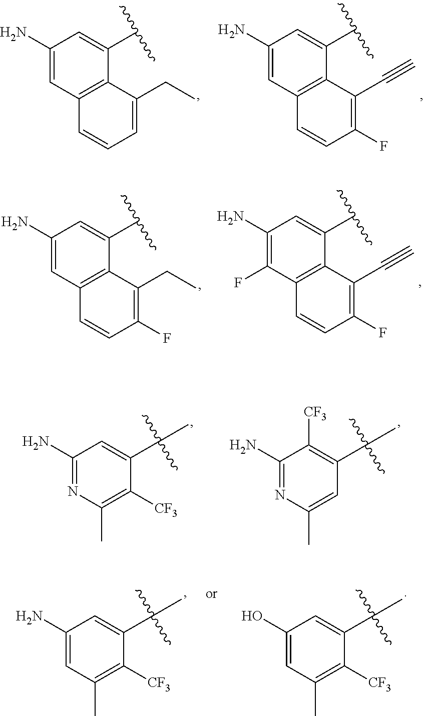

In some embodiments, the present disclosure also provides a compound of Formula III, or a pharmaceutically acceptable salt thereof:

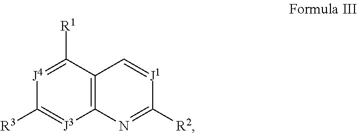

wherein:
J¹ is CR⁹ or N;
J³ is CR¹¹ or N;
J⁴ is CR¹² or N;
R¹ is hydrogen, -(L¹)ₘ₁-OR²⁰, halogen, -(L¹)ₘ₁-NR³⁰NR³¹, —C(O)—NR³⁰R³¹, optionally substituted alkyl, or an optionally substituted heterocyclic or heteroaryl ring;
R² is a ring or ring-chain structure, e.g., those having a basic functional group with a pKa of the conjugated acid of about 6 or higher, or an acylated derivative thereof (i.e., the basic functional group, such as a basic NH, is bonded with an acyl group);
R³ is an optionally substituted aryl or an optionally substituted heteroaryl,
R⁹ is hydrogen, halogen, cyano, optionally substituted C₁₋₄ alkyl (e.g., methyl, ethyl, CF₃, etc.), optionally substituted C₂₋₄ alkenyl, optionally substituted C₂₋₄ alkynyl, optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹ and R¹² at each occurrence is independently F, Cl, Br, I, CN, —OH, —C(O)NH₂, —NH₂, —NH(C₁₋₆ alkyl), —N(C₁₋₆ alkyl)(C₁₋₆ alkyl), —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), optionally substituted C₂₋₄ alkenyl, optionally substituted C₂₋₄ alkynyl, optionally substituted C₃₋₆ cycloalkyl (e.g., cyclopropyl or cyclobutyl), optionally substituted C₁₋₄ alkoxy (e.g., methoxy, ethoxy, —O—CH₂-cyclopropyl), optionally substituted C₃₋₆ cycloalkoxy (e.g., cyclopropoxy, or cyclobutoxy), optionally substituted 4-7 membered heterocyclic, or optionally substituted 4-7 membered heterocycloalkoxy;
and
   wherein:
   m1 is 0 or 1, and when m1 is 1, L¹ is an optionally substituted alkylene, an optionally substituted carbocyclylene, an optionally substituted heterocyclylene;
   R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
   R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring; or one of R³⁰ and R³¹ together with a suitable atom of L¹ and any intervening atoms form an optionally substituted heterocyclic or heteroaryl ring.

The compound of Formula III (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula III (including any of the applicable sub-formulae as described herein) can exist as a mixture of atropisomers in any ratio, including about 1:1. In some embodiments, when applicable, the compound of Formula III (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer.

Suitable R¹, R², and R³ groups for Formula III include any of those described herein having the same respective identifiers in connection with Formula I (e.g., its subformulae) in any combination. Suitable J¹ and J³ definitions for Formula III also include any of those described herein in connection with Formula I (or its sub-formulae) in any combination. For example, in some embodiments, when present, R¹¹ in Formula III is F, Cl, —CN, —OH, methoxy, ethoxy, —O—CH₂-cyclopropyl, —C(O)NHMe, CF₃, methyl, ethyl, isopropyl, or cyclopropyl. In some embodiments, when present, R¹¹ in Formula III is hydrogen. In some embodiments, when present, R¹¹ in Formula III is Br. In some embodiments, when present, R⁹ in Formula III is hydrogen. In some embodiments, when present, R¹² in Formula III is hydrogen, F, Cl, —CN, —OH, methoxy, ethoxy, —O—CH₂-cyclopropyl, —C(O)NHMe, CF₃, methyl, ethyl, isopropyl, or cyclopropyl.

In some embodiments, the compound of Formula III can have one of the following subformulae:

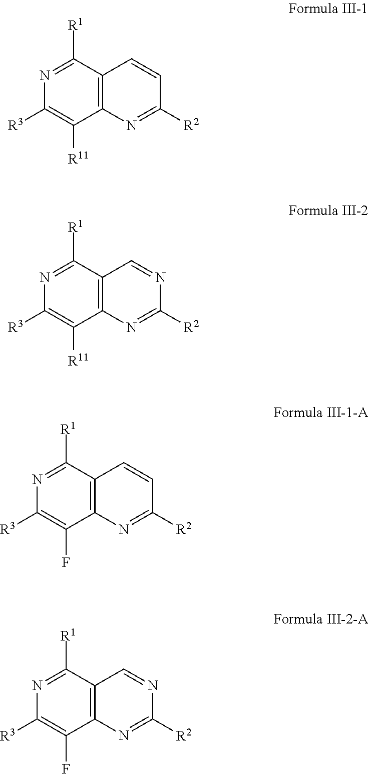

wherein R¹, R², R³, and R¹¹ include any of those defined herein in any combinations.

For example, in some embodiments, R¹ in Formula III (e.g., sub-formulae III-1, III-2, III-1-A, or III-2-A) is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted.

In some embodiments, R¹ in Formula III (e.g., sub-formulae III-1, III-2, III-1-A, or III-2-A) is selected from

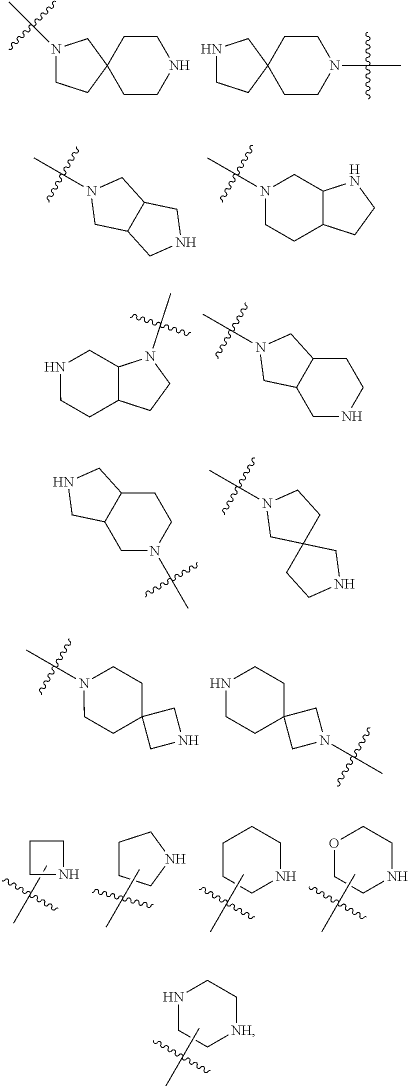

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some embodiments, R¹ in Formula III (e.g., sub-formulae III-1, III-2, III-1-A, or III-2-A) is —OR²⁰, wherein R²⁰ is a —C₁₋₆ alkylene-R¹⁰¹, wherein R¹⁰¹ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring;
R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represent a monoalkyl or dialkyl amine; or R³² and R³³ are joined to form an optionally substituted heterocyclic or heteroaryl ring; and
   R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.
   Suitable “—C₁₋₆ alkylene-” and R¹⁰¹ include any of those described herein having the same respective identifiers in connection with R²⁰ defined in Formula I (including subformulae). For example, in some embodiments, the “—C₁₋₆ alkylene-” is —CH₂—, —CH₂—CH₂—, —CH₂—CH₂—CH₂—,

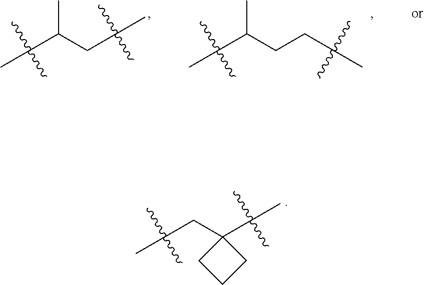

In some embodiments, R¹⁰¹ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. In some embodiments, R¹⁰¹ is a monocyclic ring selected from the following:

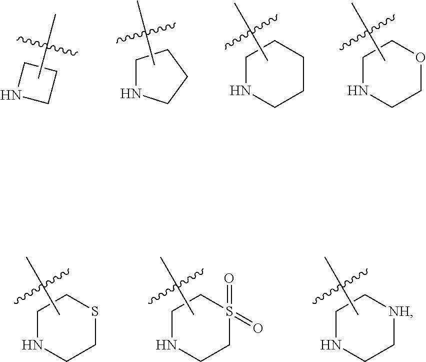

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, R¹⁰¹ is a bicyclic ring selected from the following:

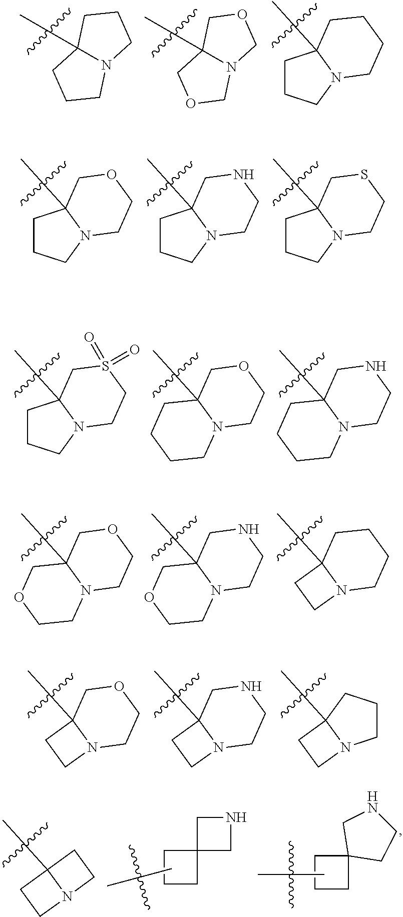

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹ in Formula III (e.g., sub-formulae III-1, III-2, III-1-A, or III-2-A) is selected from:

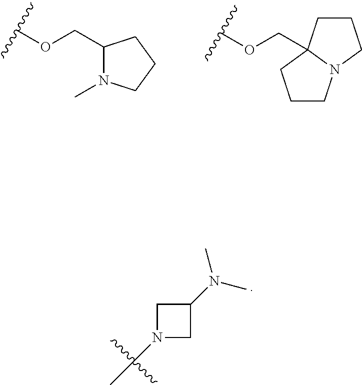

In some embodiments, R¹ in Formula III (e.g., sub-formulae III-1, III-2, III-1-A, or III-2-A) is

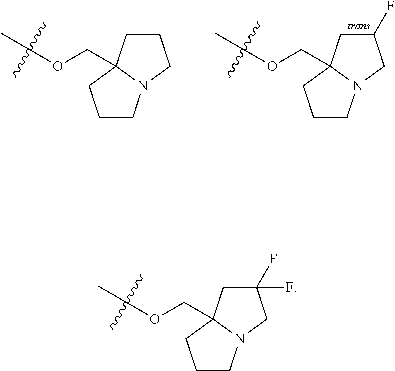 [SMILES: *OCC12CCCN1CCC2 ; *OCC12CCCN1CC(F)C2 ; *OCC12CCCN1CC(F)(F)C2]

Suitable R¹ for Formula III (e.g., Formula III-1, III-2, III-1-A, or III-2-A) include any of those described herein in connection with Formula I and those exemplified herein in the specific examples.

Suitable R² for Formula III (e.g., Formula III-1, III-2, III-1-A, or III-2-A) include any of those described herein in connection with Formula I and those exemplified herein in the specific examples. Typically, R² in Formula III does not contain a Michael acceptor, such as an alpha-beta unsaturated carbonyl structural moiety. In some embodiments, R² in Formula III (e.g., sub-formulae III-1, III-2, III-1-A, or III-2-A) is -(L²)ₘ₂-R¹⁰², wherein
m2 is 0 or 1, and when m2 is 1, L² is CH₂, O, NH, or NCH₃,
R¹⁰² is an optionally substituted 4-10 membered heterocyclic or heteroaryl ring having one or two ring nitrogen atoms.

Suitable R¹⁰² includes any of those described herein in connection with Formula I (e.g., any of its sub-formulae). In some embodiments, R¹⁰² is an optionally substituted 4-10 membered heterocyclic ring having one or two ring nitrogen atoms. In some embodiments, R¹⁰² or R² in Formula III (e.g., sub-formulae III-1. III-2, III-1-A, or III-2-A) is selected from:

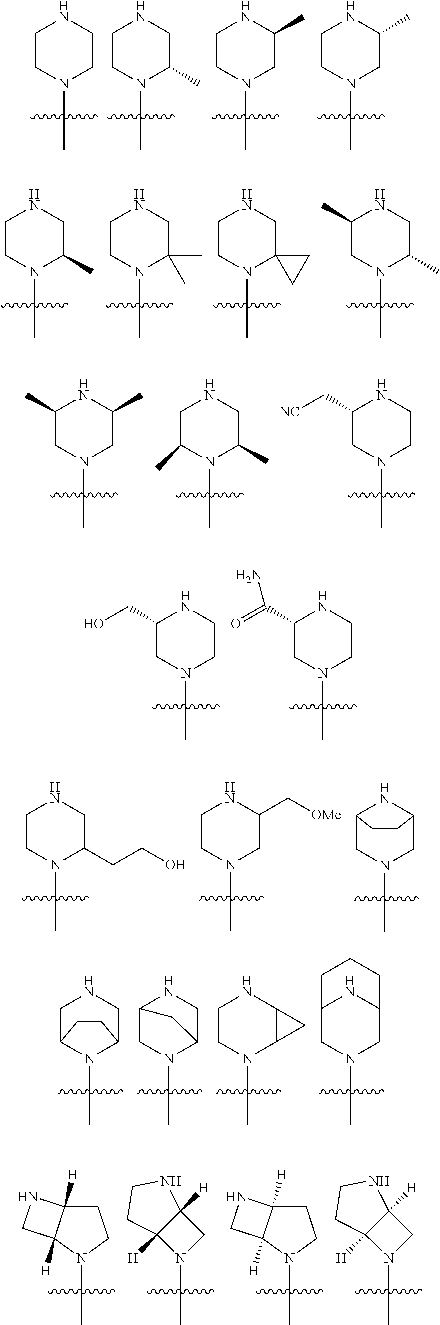

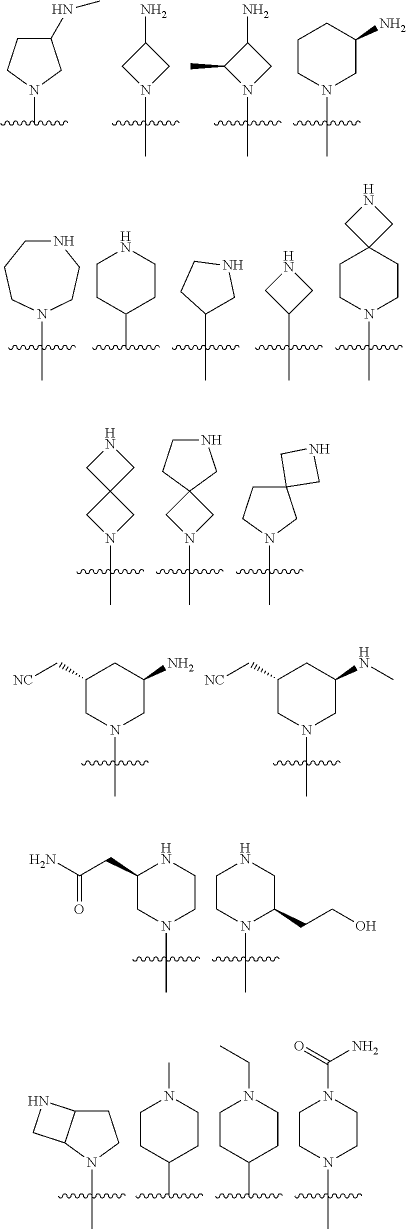

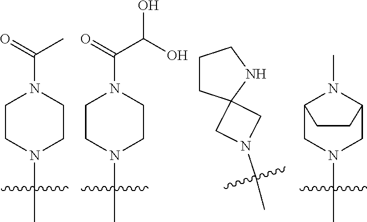

or selected from

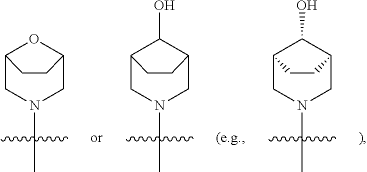 [SMILES: *N1CC2CCC(C1)O2 ; *N1CC2CCC(C1)C2O ; *N1C[C@H]2CC[C@@H](C1)[C@@H]2O]

or selected from

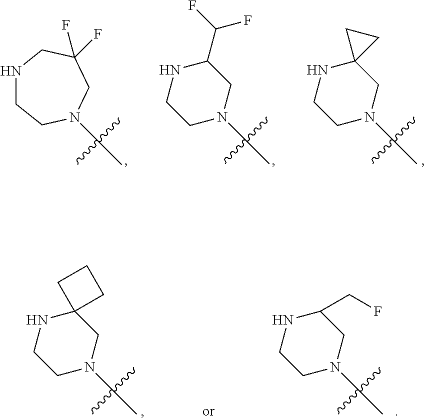 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

Suitable R³ for Formula III (e.g., sub-formulae III-1, III-2, III-1-A, or III-2-A) include any of those described herein in connection with Formula I and those exemplified herein in the specific examples. For example, in some embodiments, R³ in Formula III (e.g., sub-formulae III-1, III-2, III-1-A, or III-2-A) is a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, R³ in Formula III (e.g., sub-formulae III-1, III-2, III-1-A, or III-2-A) can be a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, R³ in Formula III (e.g., sub-formulae III-1. III-2, III-1-A, or III-2-A) is selected from

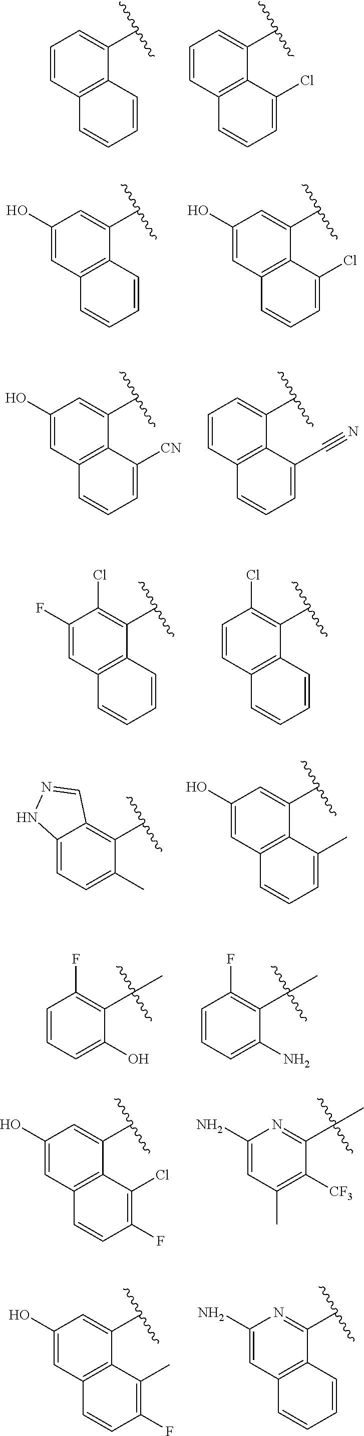

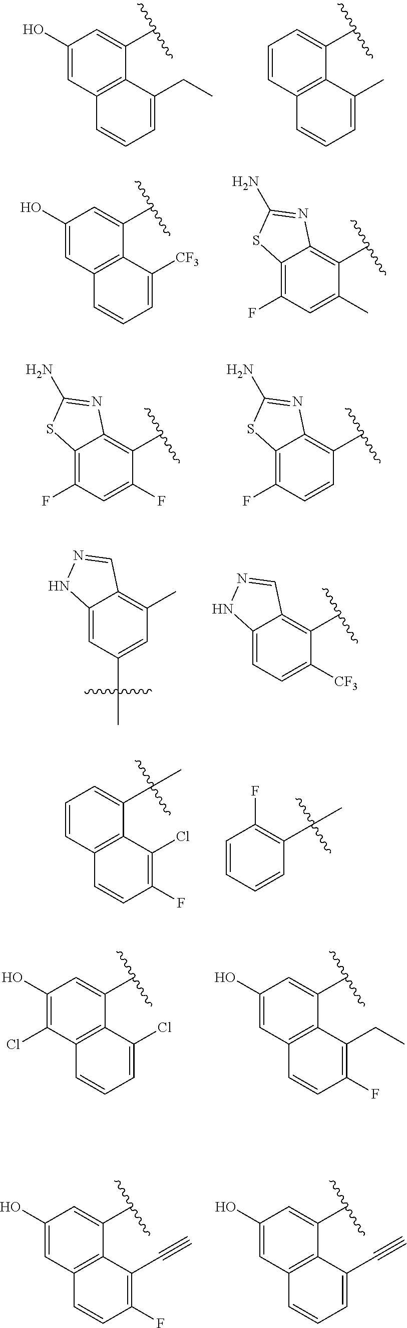

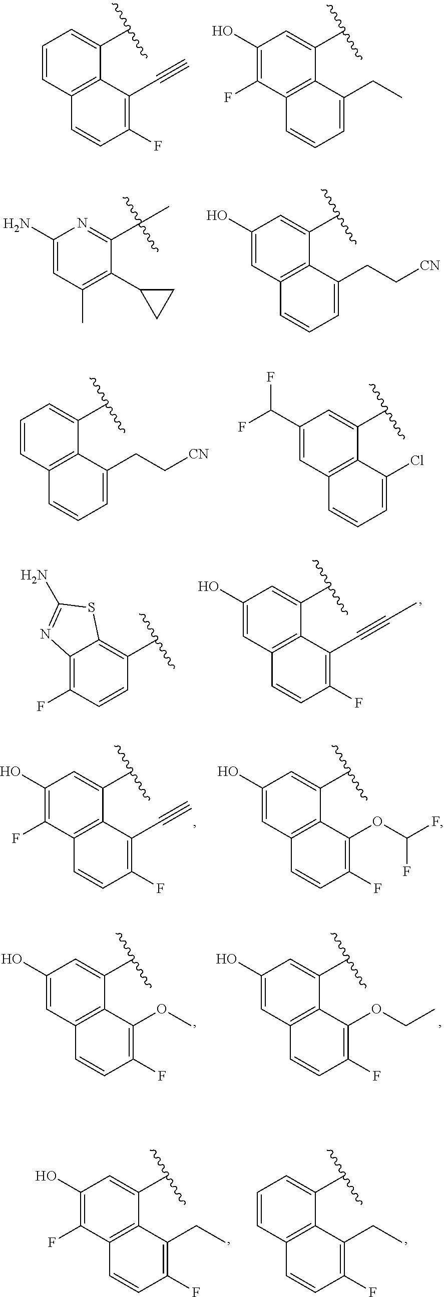

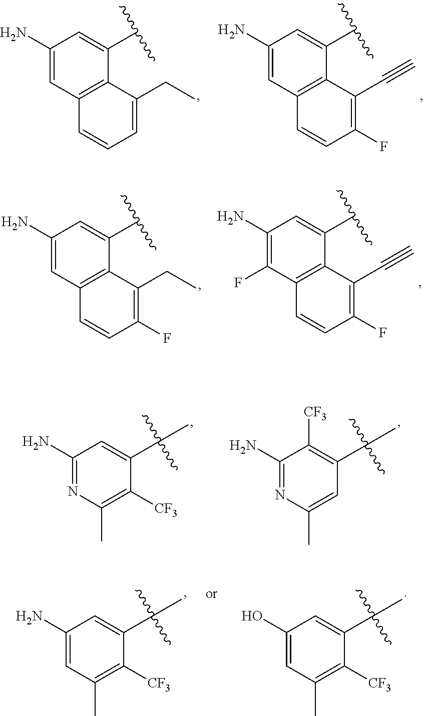

In some embodiments, the present disclosure also provides a compound of Formula IV or V, or a pharmaceutically acceptable salt thereof:

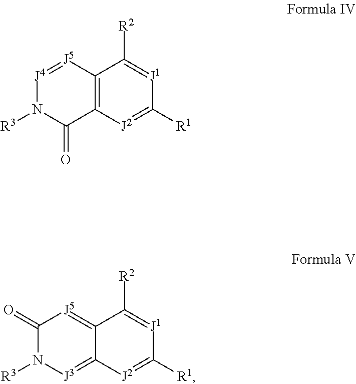

wherein:
J¹ is CR⁹ or N;
J² is CR¹⁰ or N;
J³ is CR¹¹ or N;
J⁴ is CR¹² or N;
J⁵ is CR^{12A} or N;
or J⁴ and J⁵ are joined to form an optionally substituted phenyl or optionally substituted 5, or 6-membered heteroaryl, provided that in such cases, the bond between J⁴ and J⁵ can be a single bond, for example, when J⁴ and J⁵ are joined to form a triazole ring;
R¹ is hydrogen, -(L¹)ₘ₁-OR²⁰, halogen, -(L¹)ₘ₁-NR³⁰R³¹, —C(O)—NR³⁰R³¹, optionally substituted alkyl, or an optionally substituted heterocyclic or heteroaryl ring;
R² is a ring or ring-chain structure, e.g., those having a basic functional group with a pKa of the conjugated acid of about 6 or higher, or an acylated derivative thereof (i.e., the basic functional group, such as a basic NH, is bonded with an acyl group);
R³ is an optionally substituted aryl or an optionally substituted heteroaryl,
R⁹ and R¹⁰ at each occurrence is independently hydrogen, halogen, cyano, optionally substituted C₁₋₄ alkyl (e.g., methyl, ethyl, CF₃, etc.), optionally substituted C₂₋₄ alkenyl, optionally substituted C₂₋₄ alkynyl, optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹, R¹² and R^{12A} at each occurrence is independently F, Cl, Br, I, CN, —OH, —C(O)NH₂, —NH₂, —NH(C₁₋₆ alkyl), —N(C₁₋₆ alkyl)(C₁₋₆ alkyl), —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), optionally substituted C₂₋₄ alkenyl, optionally substituted C₂₋₄ alkynyl, optionally substituted C₃₋₆ cycloalkyl (e.g., cyclopropyl or cyclobutyl), optionally substituted C₁₋₄ alkoxy (e.g., methoxy, ethoxy, —O—CH₂-cyclopropyl), optionally substituted C₃₋₆ cycloalkoxy (e.g., cyclopropoxy, or cyclobutoxy), optionally substituted 4-7 membered heterocyclic, or optionally substituted 4-7 membered heterocycloalkoxy;
or R¹² and R^{12A} are joined to form a 5-7 membered ring structure; and
   wherein:
   m1 is 0 or 1, and when m1 is 1, L¹ is an optionally substituted alkylene, an optionally substituted carbocyclylene, an optionally substituted heterocyclylene;
   R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
   R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring; or one of R³⁰ and R³¹ together with a suitable atom of L¹ and any intervening atoms form an optionally substituted heterocyclic or heteroaryl ring.

The compound of Formula IV or V (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula IV or V (including any of the applicable sub-formulae as described herein) can exist as a mixture of atropisomers in any ratio, including about 1:1. In some embodiments, when applicable, the compound of Formula IV or V (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer.

Suitable R¹, R², and R³ groups for Formula IV or V include any of those described herein having the same respective identifiers in connection with Formula I (e.g., its subformulae) in any combination. Suitable J¹, J², J³, J⁴, and J⁵ definitions for Formula IV or V also include any of those described herein in connection with Formula I (or its sub-formulae) in any combination. For example, in some embodiments, J¹ and J² are N. In some embodiments, when present, R⁹ in Formula IV or V is hydrogen. In some embodiments, J³ in Formula V is CR¹¹, wherein R¹¹ is F, Cl, —CN, —OH, methoxy, ethoxy, —O—CH₂-cyclopropyl, —C(O)NHMe, CF₃, methyl, ethyl, isopropyl, or cyclopropyl.

In some embodiments, the compound of Formula IV or V can have one of the following subformulae:

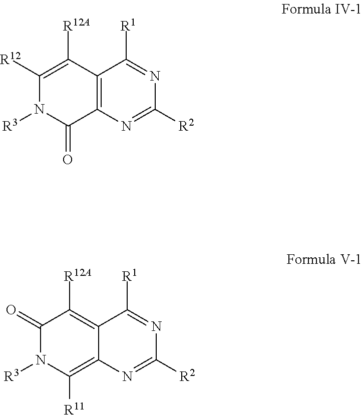

wherein R¹, R², R³, R¹¹, R¹², and R^{12A} include any of those defined herein in any combinations.

For example, in some embodiments, when present, R¹¹ in Formula V (e.g., sub-formulae V-1) is hydrogen, F, Cl, or methyl. In some embodiments, when present, R¹² in Formula IV (e.g., sub-formulae IV-1) is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, when present, R^{12A} in Formula IV or V (e.g., sub-formulae IV-1 or V-1) is hydrogen, F, Cl, —CN, —OH, methoxy, ethoxy, —O—CH₂-cyclopropyl, —C(O)NHMe, CF₃, methyl, ethyl, isopropyl, or cyclopropyl. In some embodiments, R^{12A} in Formula IV or V (e.g., sub-formulae IV-1 or V-1) can be H or C₁₋₄ alkyl optionally substituted with F, such as methyl. In some embodiments, R^{12A} in Formula IV or V (e.g., sub-formulae IV-1 or V-1) can be Cl or methoxy. In some embodiments, R^{12A} in Formula IV or V (e.g., sub-formulae IV-1 or V-1) can be ethyl or difluoromethyl. In some embodiments, R^{12A} in Formula IV or V (e.g., sub-formulae IV-1 or V-1) is OH. Suitable R^{12A} for Formula IV or V (e.g., sub-formulae IV-1 or V-1) also include those exemplified herein in the specific examples.

In some embodiments, R¹ in Formula IV or V (e.g., sub-formulae IV-1 or V-1) is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted.

In some embodiments, R¹ in Formula IV or V (e.g., sub-formulae IV-1 or V-1) is selected from

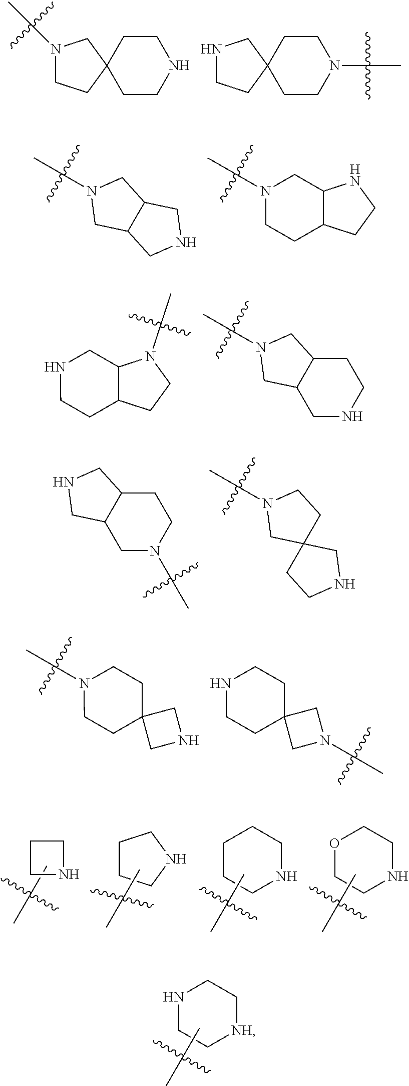

each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ N(C₁₋₄ alkyl)(C₁₋₄ alkyl), —(CH₂)ₓ cyclopropyl, —(CH₂)ₓ-cyclobutyl, and —(CH₂)-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some embodiments, R¹ in Formula IV or V (e.g., sub-formulae IV-1 or V-1) is —OR²⁰ wherein R²⁰ is a —C₁₋₆ alkylene-R¹⁰¹, wherein R¹⁰¹ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring;
R³² and R³³ are independently hydrogen, a nitrogen protecting group, an optionally substituted C₁₋₆ alkyl, an optionally substituted carbocyclic ring, or an optionally substituted heterocyclic ring; or NR³²R³³ represent a monoalkyl or dialkyl amine; or R³² and R³³ are joined to form an optionally substituted heterocyclic or heteroaryl ring; and
   R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.
   Suitable “—C₁₋₆ alkylene-” and R¹⁰¹ include any of those described herein having the same respective identifiers in connection with R²⁰ defined in Formula I (including subformulae). For example, in some embodiments, the “—C₁₋₆ alkylene-” is —CH₂—, —CH₂—CH₂—, —CH₂—CH₂—CH₂—,

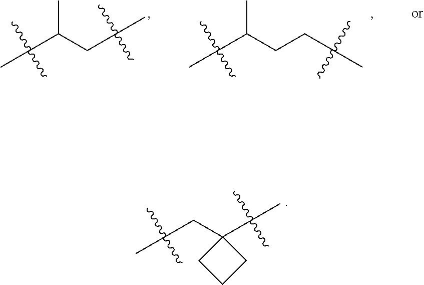

In some embodiments, R¹⁰¹ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted. In some embodiments, R¹⁰¹ is a monocyclic ring selected from the following:

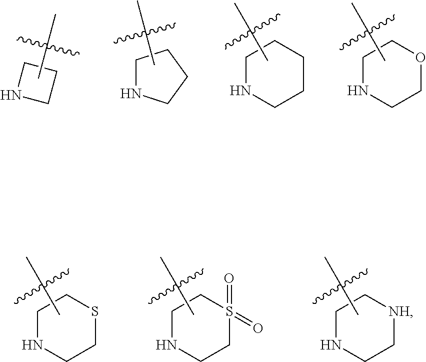

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, R¹⁰¹ is a bicyclic ring selected from the following.

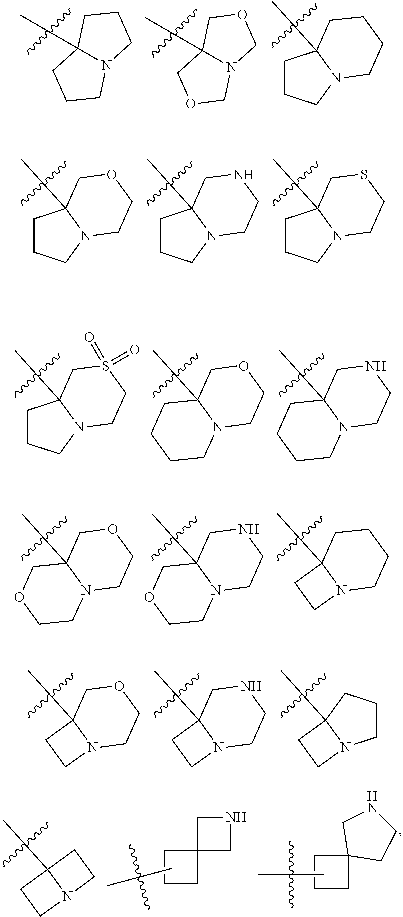

each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹ in Formula IV or V (e.g., sub-formulae IV-1 or V-1) is selected from:

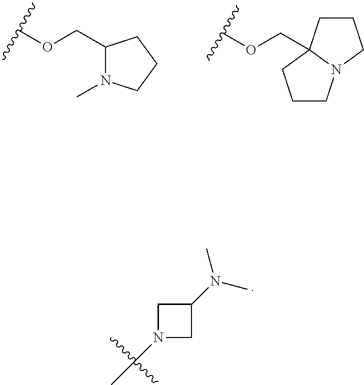

In some embodiments, R¹ in Formula IV or V (e.g., sub-formulae IV-1 or V-1) is

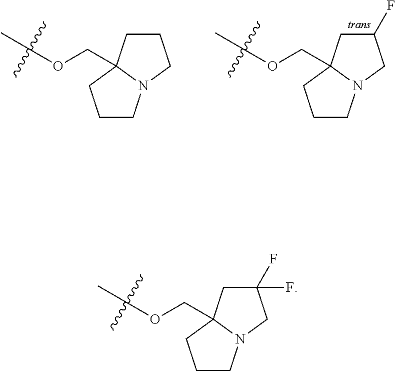 [SMILES: *OCC12CCCN1CCC2 ; *OCC12CCCN1CC(F)C2 ; *OCC12CCCN1CC(F)(F)C2]

Suitable R¹ for Formula IV or V (e.g., sub-formulae IV-1 or V-1) include any of those described herein in connection with Formula I and those exemplified herein in the specific examples.

Suitable R² for Formula IV or V (e.g., sub-formulae IV-1 or V-1) include any of those described herein in connection with Formula I and those exemplified herein in the specific examples. Typically, R² in Formula IV or V (e.g., sub-formulae IV-1 or V-1) does not contain a Michael acceptor, such as an alpha-beta unsaturated carbonyl structural moiety. In some embodiments, R² in Formula IV or V (e.g., sub-formulae IV-1 or V-1) is -(L²)ₘ₂-R¹⁰², wherein

m2 is 0 or 1, and when m2 is 1, L² is CH₂, O, NH, or NCH₃,

R¹⁰² is an optionally substituted 4-10 membered heterocyclic or heteroaryl ring having one or two ring nitrogen atoms.

Suitable R¹⁰² includes any of those described herein in connection with Formula I (e.g., any of its sub-formulae). In some embodiments, R¹⁰² is an optionally substituted 4-10 membered heterocyclic ring having one or two ring nitrogen atoms. In some embodiments, R¹⁰² or R² in Formula IV or V (e.g., sub-formulae IV-1 or V-1) is selected from:

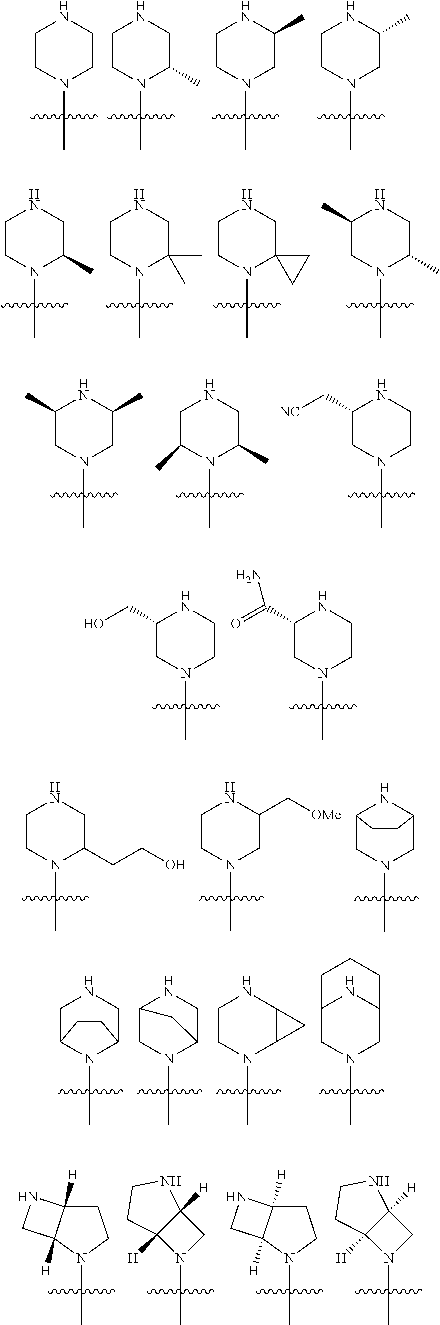

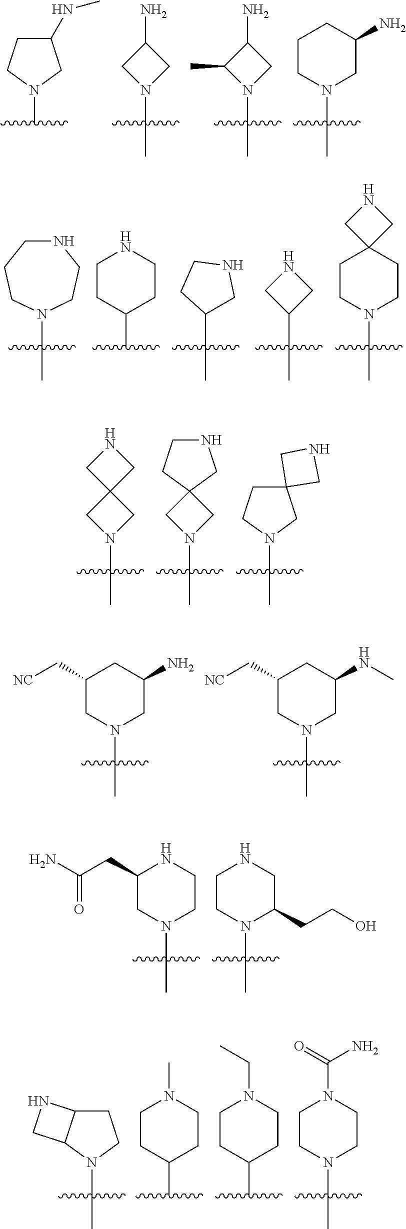

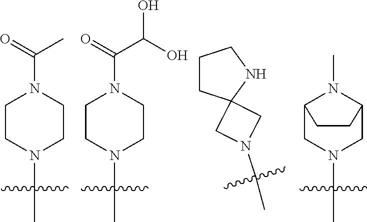

or selected from

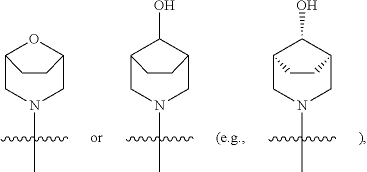 [SMILES: *N1CC2CCC(C1)O2 ; *N1CC2CCC(C1)C2O ; *N1C[C@H]2CC[C@@H](C1)[C@@H]2O]

or selected from

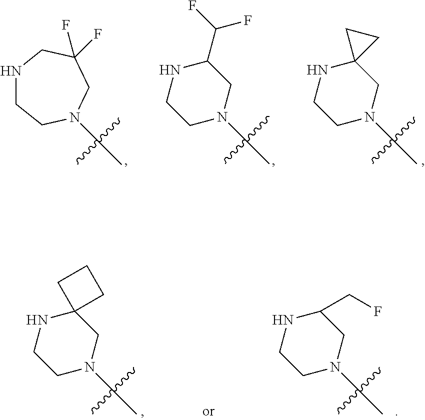 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

Suitable R³ for Formula IV or V (e.g., sub-formulae IV-1 or V-1) include any of those described herein in connection with Formula I and those exemplified herein in the specific examples. For example, in some embodiments, R³ in Formula IV or V (e.g., sub-formulae IV-1 or V-1) is a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, R³ in Formula IV or V (e.g., sub-formulae IV-1 or V-1) can be a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂. In some embodiments, R³ in Formula IV or V (e.g., sub-formulae IV-1 or V-1) is selected from

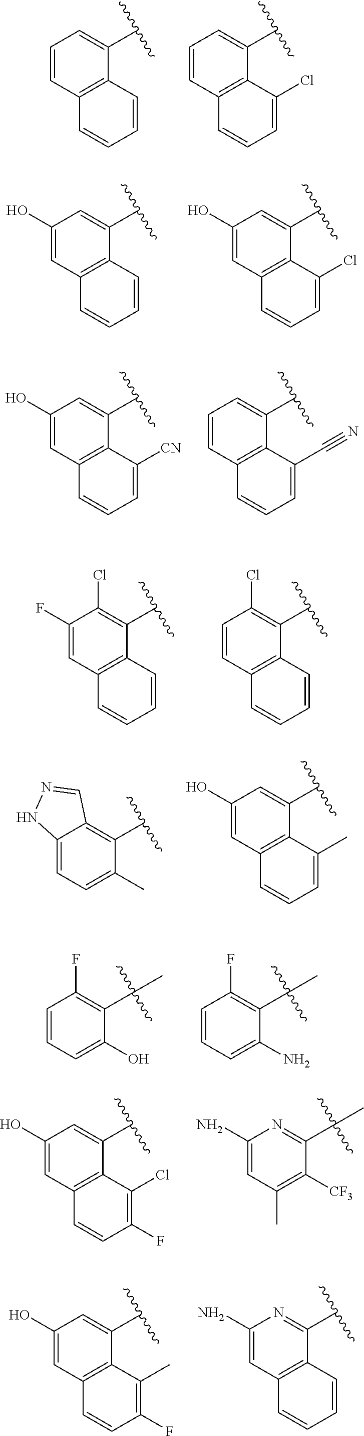

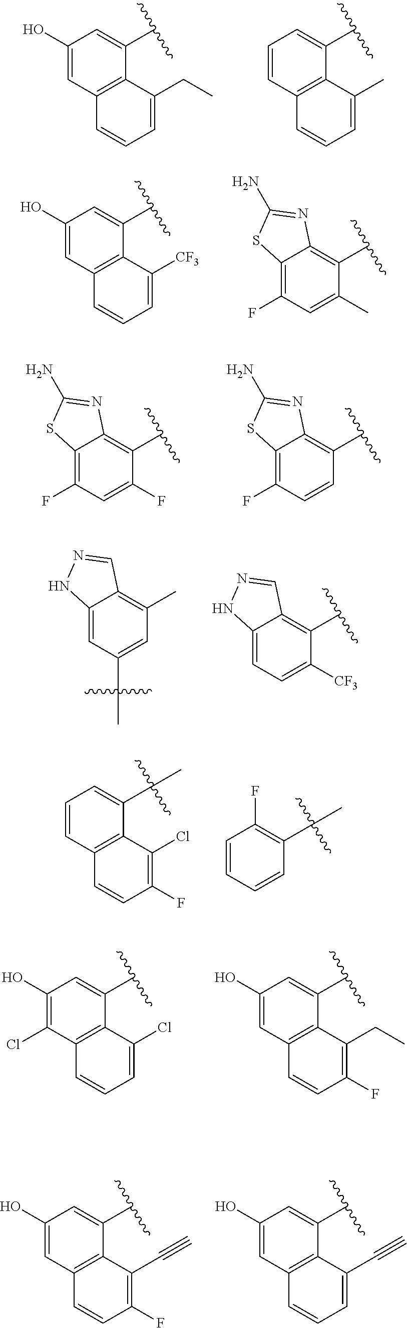

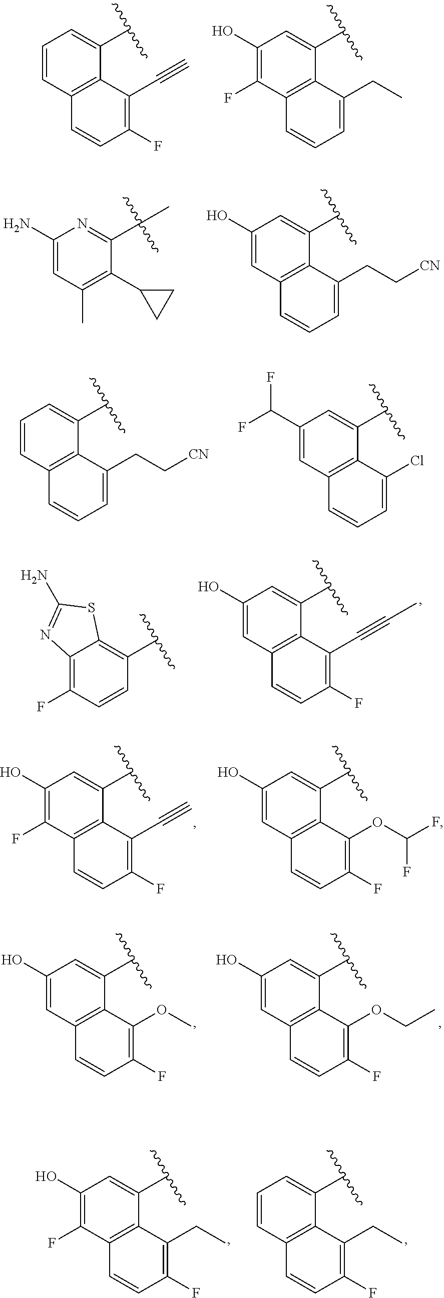

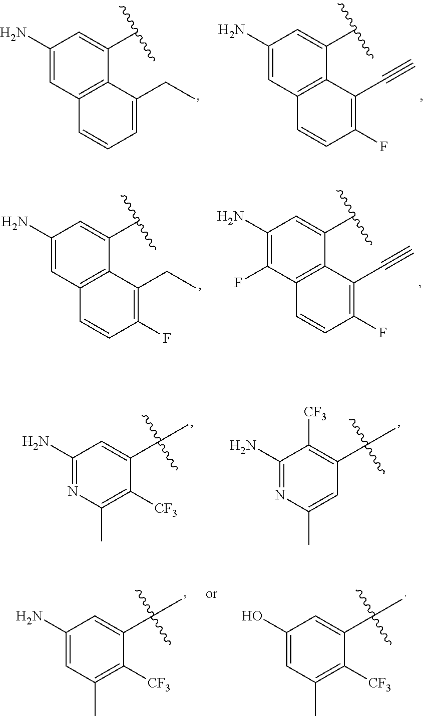

In some embodiments, the present disclosure also provides a compound selected from the following Compound Nos. I-247, or a pharmaceutically acceptable salt thereof:

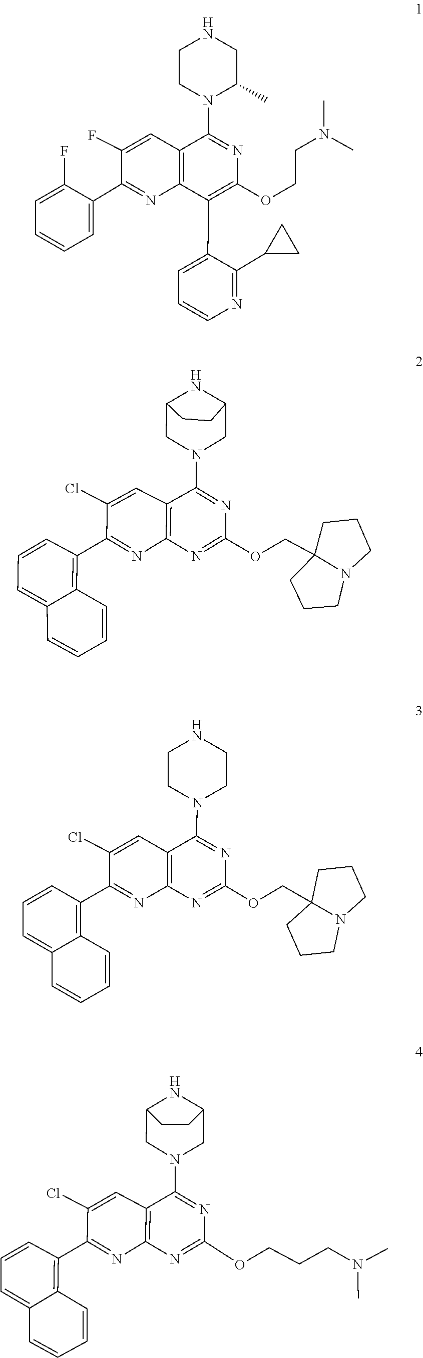 [SMILES: C[C@H]1CNCCN1c1nc(OCCN(C)C)c(-c2cccnc2C2CC2)c2nc(-c3ccccc3F)c(F)cc12 ; Clc1cc2c(N3CC4CCC(C3)N4)nc(OCC34CCCN3CCC4)nc2nc1-c1cccc2ccccc12 ; Clc1cc2c(N3CCNCC3)nc(OCC34CCCN3CCC4)nc2nc1-c1cccc2ccccc12 ; CN(C)CCCOc1nc(N2CC3CCC(C2)N3)c2cc(Cl)c(-c3cccc4ccccc34)nc2n1]

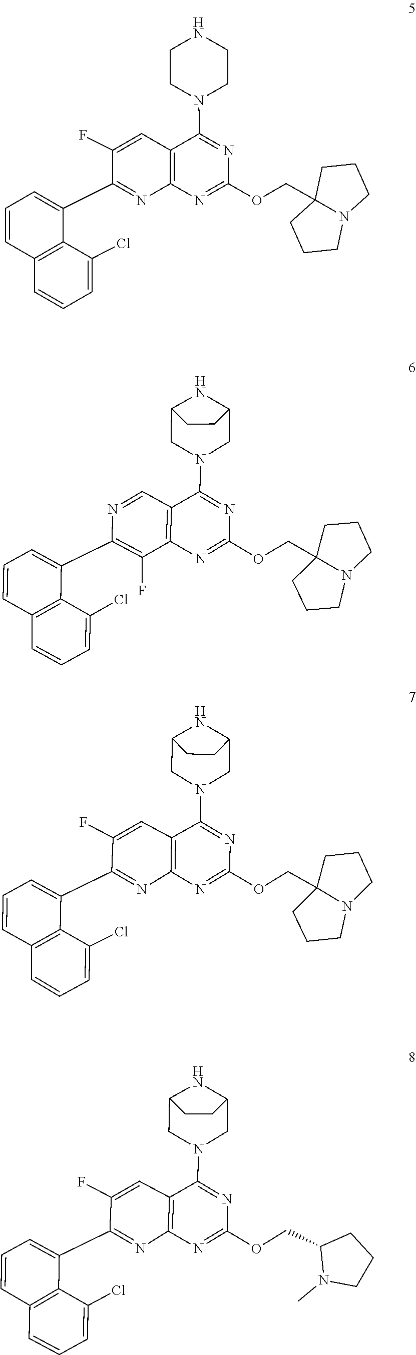 [SMILES: Fc1cc2c(N3CCNCC3)nc(OCC34CCCN3CCC4)nc2nc1-c1cccc2cccc(Cl)c12 ; Fc1c(-c2cccc3cccc(Cl)c23)ncc2c(N3CC4CCC(C3)N4)nc(OCC34CCCN3CCC4)nc12 ; Fc1cc2c(N3CC4CCC(C3)N4)nc(OCC34CCCN3CCC4)nc2nc1-c1cccc2cccc(Cl)c12 ; CN1CCC[C@H]1COc1nc(N2CC3CCC(C2)N3)c2cc(F)c(-c3cccc4cccc(Cl)c34)nc2n1]

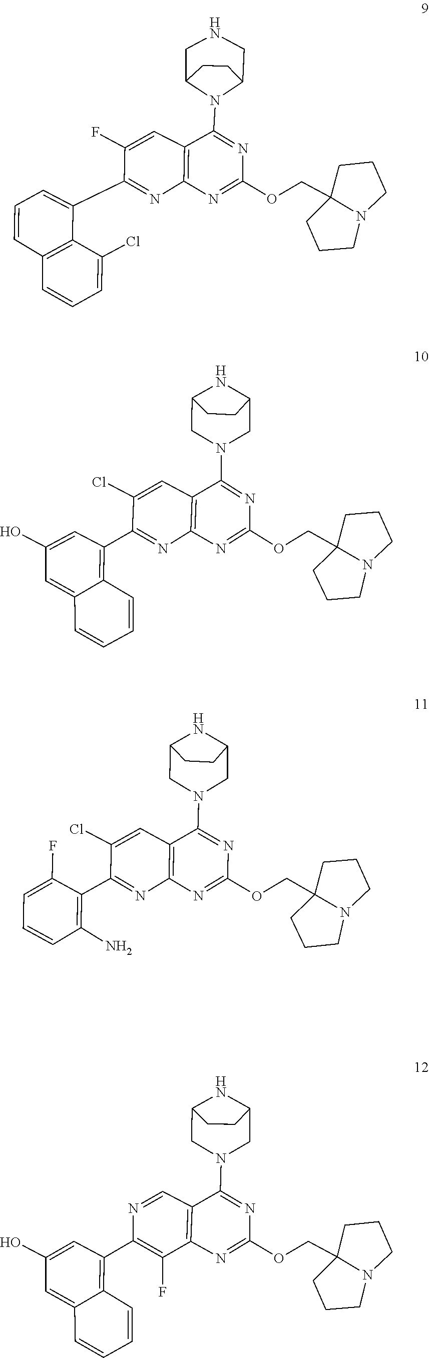 [SMILES: Fc1cc2c(N3C4CCC3CNC4)nc(OCC34CCCN3CCC4)nc2nc1-c1cccc2cccc(Cl)c12 ; Oc1cc(-c2nc3nc(OCC45CCCN4CCC5)nc(N4CC5CCC(C4)N5)c3cc2Cl)c2ccccc2c1 ; Nc1cccc(F)c1-c1nc2nc(OCC34CCCN3CCC4)nc(N3CC4CCC(C3)N4)c2cc1Cl ; Oc1cc(-c2ncc3c(N4CC5CCC(C4)N5)nc(OCC45CCCN4CCC5)nc3c2F)c2ccccc2c1]

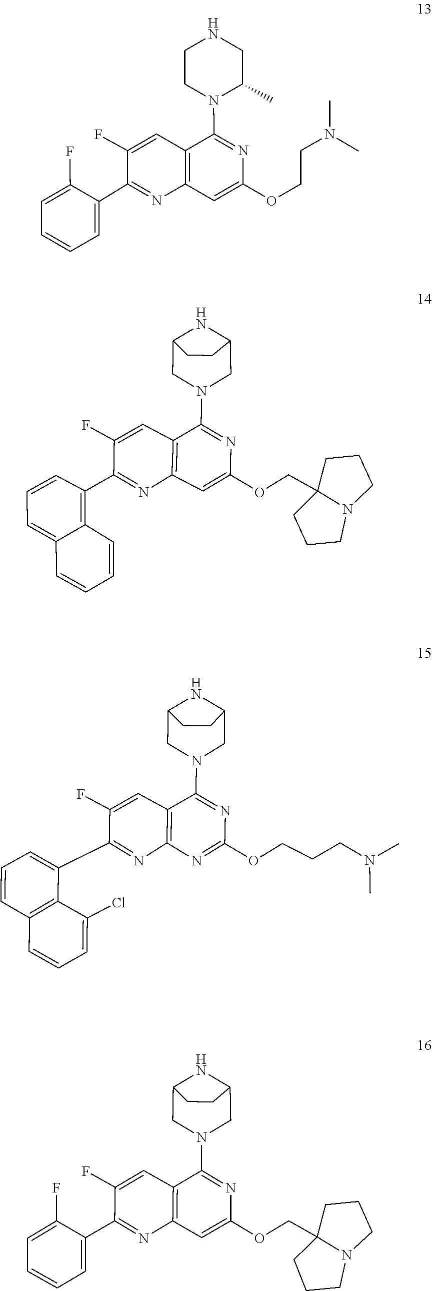 [SMILES: C[C@H]1CNCCN1c1nc(OCCN(C)C)cc2nc(-c3ccccc3F)c(F)cc12 ; Fc1cc2c(N3CC4CCC(C3)N4)nc(OCC34CCCN3CCC4)cc2nc1-c1cccc2ccccc12 ; CN(C)CCCOc1nc(N2CC3CCC(C2)N3)c2cc(F)c(-c3cccc4cccc(Cl)c34)nc2n1 ; Fc1ccccc1-c1nc2cc(OCC34CCCN3CCC4)nc(N3CC4CCC(C3)N4)c2cc1F]

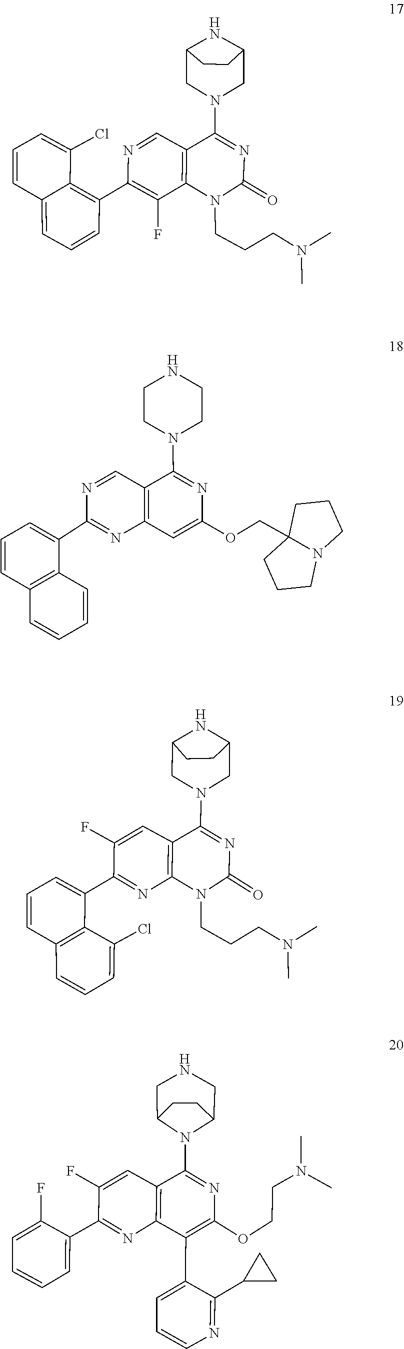 [SMILES: CN(C)CCCn1c(=O)nc(N2CC3CCC(C2)N3)c2cnc(-c3cccc4cccc(Cl)c34)c(F)c21 ; c1ccc2c(-c3ncc4c(N5CCNCC5)nc(OCC56CCCN5CCC6)cc4n3)cccc2c1 ; CN(C)CCCn1c(=O)nc(N2CC3CCC(C2)N3)c2cc(F)c(-c3cccc4cccc(Cl)c34)nc21 ; CN(C)CCOc1nc(N2C3CCC2CNC3)c2cc(F)c(-c3ccccc3F)nc2c1-c1cccnc1C1CC1]

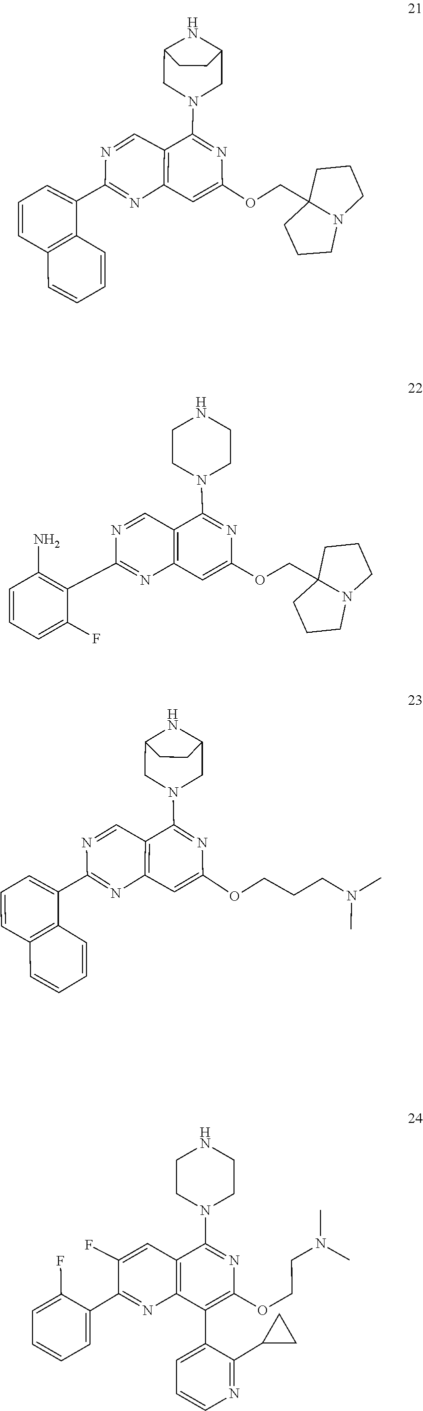 [SMILES: c1ccc2c(-c3ncc4c(N5CC6CCC(C5)N6)nc(OCC56CCCN5CCC6)cc4n3)cccc2c1 ; Nc1cccc(F)c1-c1ncc2c(N3CCNCC3)nc(OCC34CCCN3CCC4)cc2n1 ; CN(C)CCCOc1cc2nc(-c3cccc4ccccc34)ncc2c(N2CC3CCC(C2)N3)n1 ; CN(C)CCOc1nc(N2CCNCC2)c2cc(F)c(-c3ccccc3F)nc2c1-c1cccnc1C1CC1]

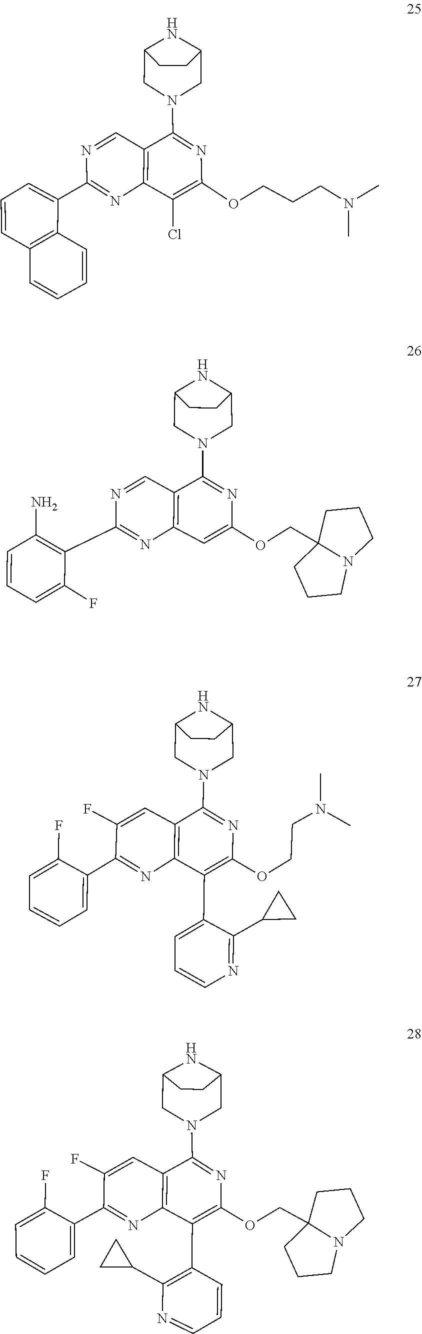 [SMILES: CN(C)CCCOc1nc(N2CC3CCC(C2)N3)c2cnc(-c3cccc4ccccc34)nc2c1Cl ; Nc1cccc(F)c1-c1ncc2c(N3CC4CCC(C3)N4)nc(OCC34CCCN3CCC4)cc2n1 ; CN(C)CCOc1nc(N2CC3CCC(C2)N3)c2cc(F)c(-c3ccccc3F)nc2c1-c1cccnc1C1CC1 ; Fc1ccccc1-c1nc2c(-c3cccnc3C3CC3)c(OCC34CCCN3CCC4)nc(N3CC4CCC(C3)N4)c2cc1F]

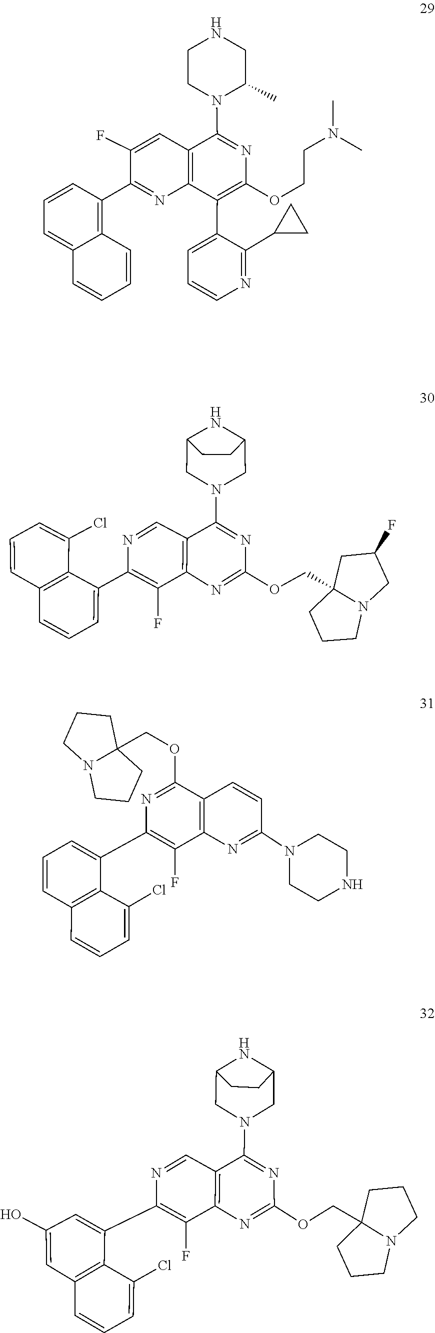 [SMILES: C[C@H]1CNCCN1c1nc(OCCN(C)C)c(-c2cccnc2C2CC2)c2nc(-c3cccc4ccccc34)c(F)cc12 ; Fc1c(-c2cccc3cccc(Cl)c23)ncc2c(N3CC4CCC(C3)N4)nc(OC[C@@]34CCCN3C[C@H](F)C4)nc12 ; Fc1c(-c2cccc3cccc(Cl)c23)nc(OCC23CCCN2CCC3)c2ccc(N3CCNCC3)nc12 ; Oc1cc(-c2ncc3c(N4CC5CCC(C4)N5)nc(OCC45CCCN4CCC5)nc3c2F)c2c(Cl)cccc2c1]

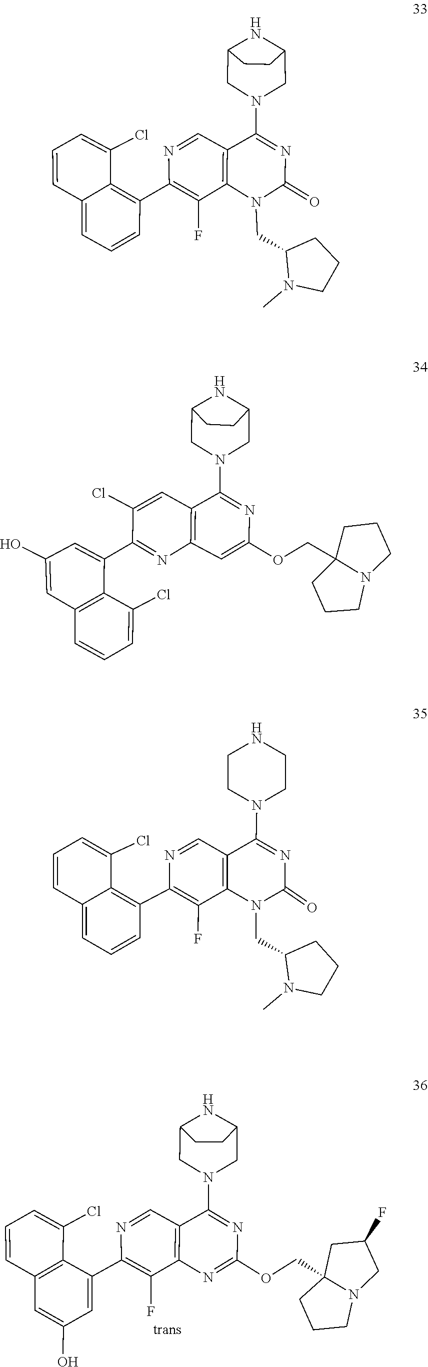 [SMILES: CN1CCC[C@H]1Cn1c(=O)nc(N2CC3CCC(C2)N3)c2cnc(-c3cccc4cccc(Cl)c34)c(F)c21 ; Oc1cc(-c2nc3cc(OCC45CCCN4CCC5)nc(N4CC5CCC(C4)N5)c3cc2Cl)c2c(Cl)cccc2c1 ; CN1CCC[C@H]1Cn1c(=O)nc(N2CCNCC2)c2cnc(-c3cccc4cccc(Cl)c34)c(F)c21 ; Oc1cc(-c2ncc3c(N4CC5CCC(C4)N5)nc(OC[C@@]45CCCN4C[C@H](F)C5)nc3c2F)c2c(Cl)cccc2c1]

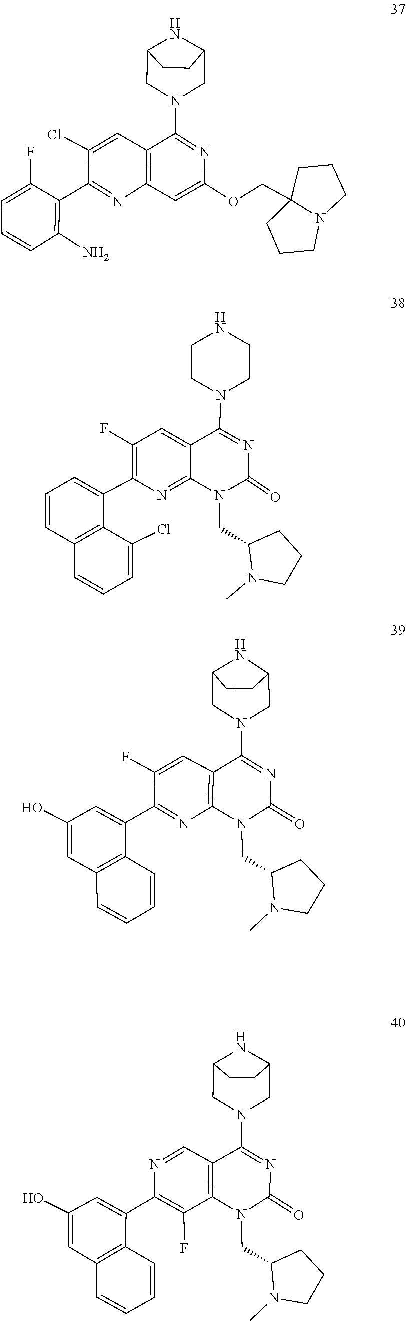 [SMILES: Nc1cccc(F)c1-c1nc2cc(OCC34CCCN3CCC4)nc(N3CC4CCC(C3)N4)c2cc1Cl ; CN1CCC[C@H]1Cn1c(=O)nc(N2CCNCC2)c2cc(F)c(-c3cccc4cccc(Cl)c34)nc21 ; CN1CCC[C@H]1Cn1c(=O)nc(N2CC3CCC(C2)N3)c2cc(F)c(-c3cc(O)cc4ccccc34)nc21 ; CN1CCC[C@H]1Cn1c(=O)nc(N2CC3CCC(C2)N3)c2cnc(-c3cc(O)cc4ccccc34)c(F)c21]

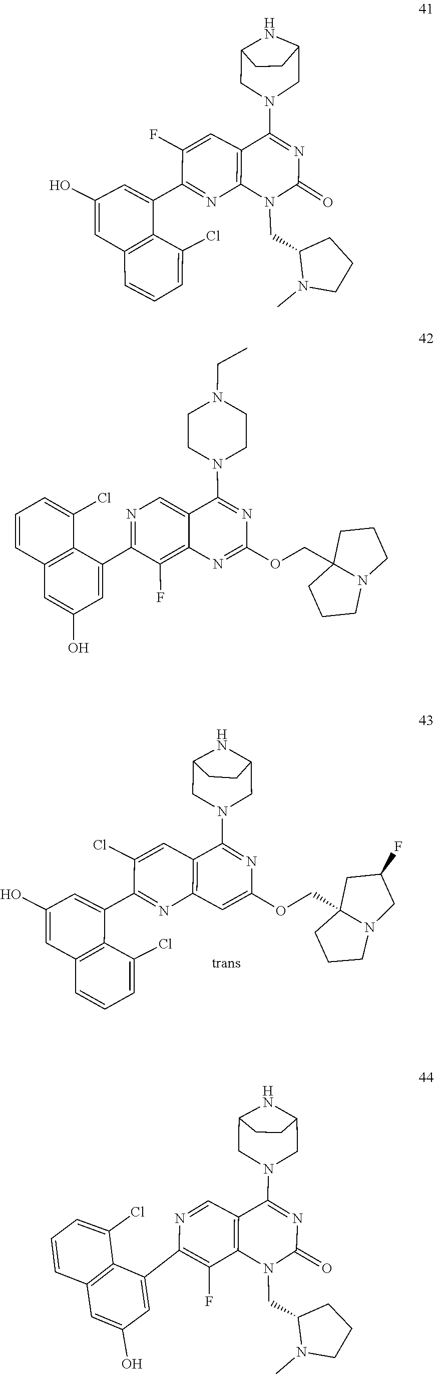 [SMILES: CN1CCC[C@H]1Cn1c(=O)nc(N2CC3CCC(C2)N3)c2cc(F)c(-c3cc(O)cc4cccc(Cl)c34)nc21 ; CCN1CCN(c2nc(OCC34CCCN3CCC4)nc3c(F)c(-c4cc(O)cc5cccc(Cl)c45)ncc23)CC1 ; Oc1cc(-c2nc3cc(OC[C@@]45CCCN4C[C@H](F)C5)nc(N4CC5CCC(C4)N5)c3cc2Cl)c2c(Cl)cccc2c1 ; CN1CCC[C@H]1Cn1c(=O)nc(N2CC3CCC(C2)N3)c2cnc(-c3cc(O)cc4cccc(Cl)c34)c(F)c21]

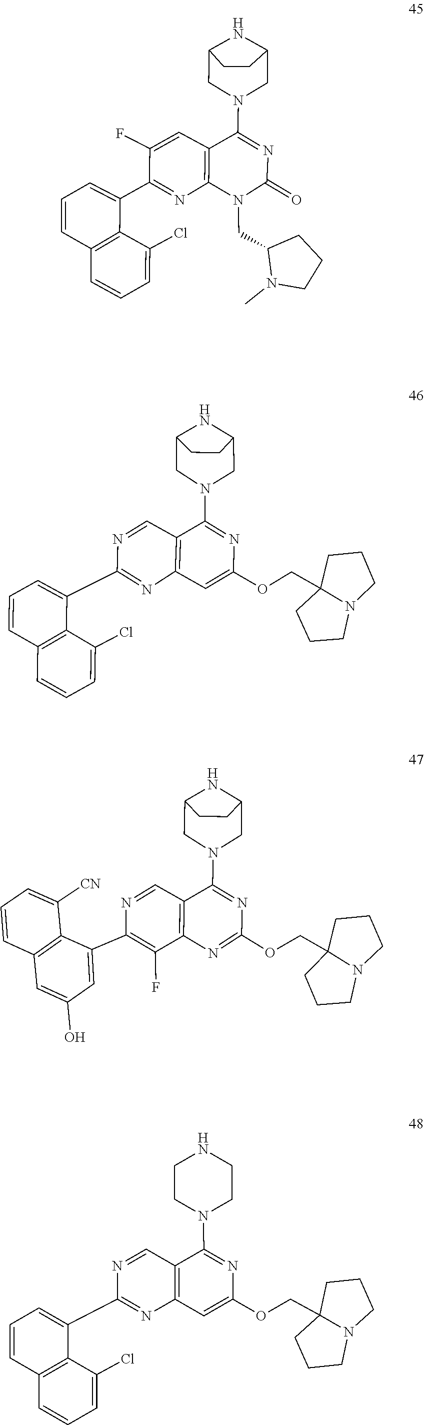 [SMILES: CN1CCC[C@H]1Cn1c(=O)nc(N2CC3CCC(C2)N3)c2cc(F)c(-c3cccc4cccc(Cl)c34)nc21 ; Clc1cccc2cccc(-c3ncc4c(N5CC6CCC(C5)N6)nc(OCC56CCCN5CCC6)cc4n3)c12 ; N#Cc1cccc2cc(O)cc(-c3ncc4c(N5CC6CCC(C5)N6)nc(OCC56CCCN5CCC6)nc4c3F)c12 ; Clc1cccc2cccc(-c3ncc4c(N5CCNCC5)nc(OCC56CCCN5CCC6)cc4n3)c12]

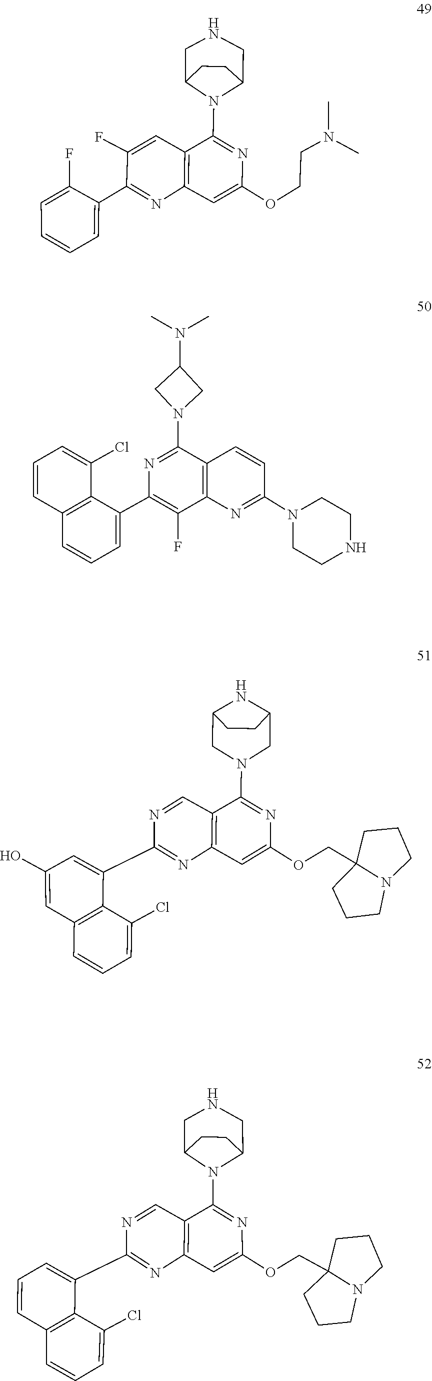 [SMILES: CN(C)CCOc1cc2nc(-c3ccccc3F)c(F)cc2c(N2C3CCC2CNC3)n1 ; CN(C)C1CN(c2nc(-c3cccc4cccc(Cl)c34)c(F)c3nc(N4CCNCC4)ccc23)C1 ; Oc1cc(-c2ncc3c(N4CC5CCC(C4)N5)nc(OCC45CCCN4CCC5)cc3n2)c2c(Cl)cccc2c1 ; Clc1cccc2cccc(-c3ncc4c(N5C6CCC5CNC6)nc(OCC56CCCN5CCC6)cc4n3)c12]

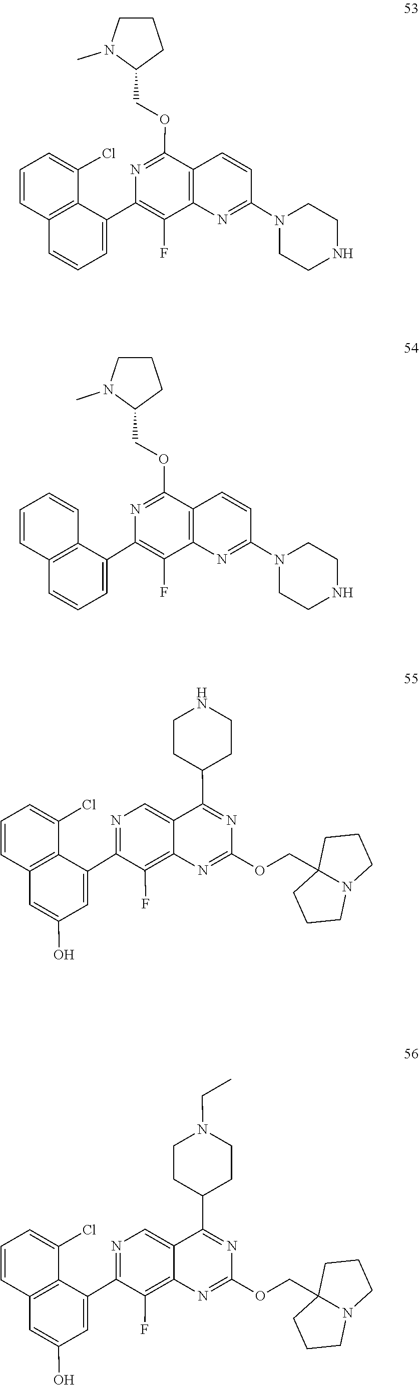 [SMILES: CN1CCC[C@@H]1COc1nc(-c2cccc3cccc(Cl)c23)c(F)c2nc(N3CCNCC3)ccc12 ; CN1CCC[C@@H]1COc1nc(-c2cccc3ccccc23)c(F)c2nc(N3CCNCC3)ccc12 ; Oc1cc(-c2ncc3c(C4CCNCC4)nc(OCC45CCCN4CCC5)nc3c2F)c2c(Cl)cccc2c1 ; CCN1CCC(c2nc(OCC34CCCN3CCC4)nc3c(F)c(-c4cc(O)cc5cccc(Cl)c45)ncc23)CC1]

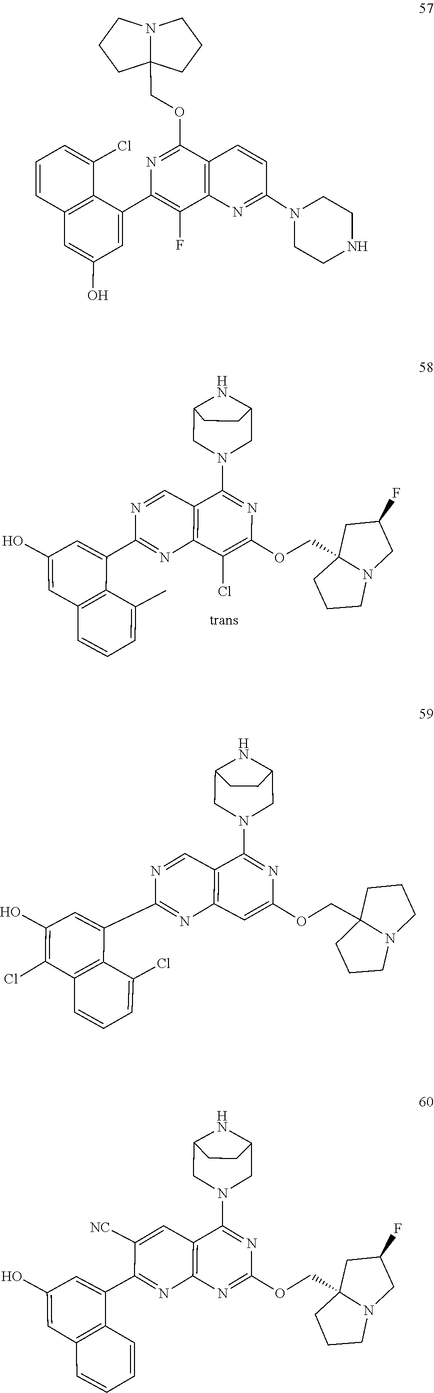 [SMILES: Oc1cc(-c2nc(OCC34CCCN3CCC4)c3ccc(N4CCNCC4)nc3c2F)c2c(Cl)cccc2c1 ; Cc1cccc2cc(O)cc(-c3ncc4c(N5CC6CCC(C5)N6)nc(OC[C@@]56CCCN5C[C@H](F)C6)c(Cl)c4n3)c12 ; Oc1cc(-c2ncc3c(N4CC5CCC(C4)N5)nc(OCC45CCCN4CCC5)cc3n2)c2c(Cl)cccc2c1Cl ; N#Cc1cc2c(N3CC4CCC(C3)N4)nc(OC[C@@]34CCCN3C[C@H](F)C4)nc2nc1-c1cc(O)cc2ccccc12]

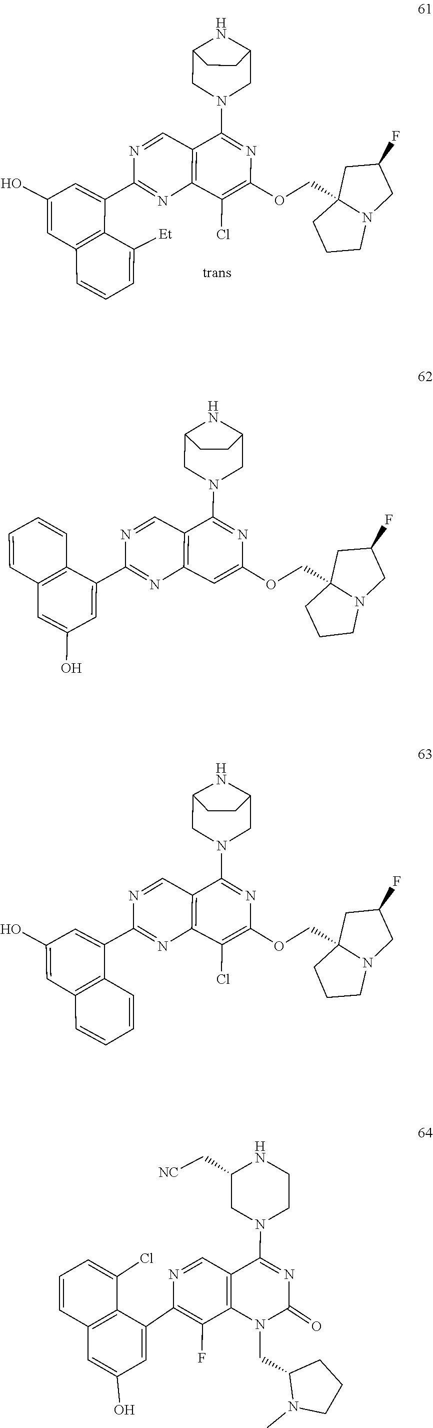 [SMILES: CCc1cccc2cc(O)cc(-c3ncc4c(N5CC6CCC(C5)N6)nc(OC[C@@]56CCCN5C[C@H](F)C6)c(Cl)c4n3)c12 ; Oc1cc(-c2ncc3c(N4CC5CCC(C4)N5)nc(OC[C@@]45CCCN4C[C@H](F)C5)cc3n2)c2ccccc2c1 ; Oc1cc(-c2ncc3c(N4CC5CCC(C4)N5)nc(OC[C@@]45CCCN4C[C@H](F)C5)c(Cl)c3n2)c2ccccc2c1 ; CN1CCC[C@H]1Cn1c(=O)nc(N2CCN[C@@H](CC#N)C2)c2cnc(-c3cc(O)cc4cccc(Cl)c34)c(F)c21]

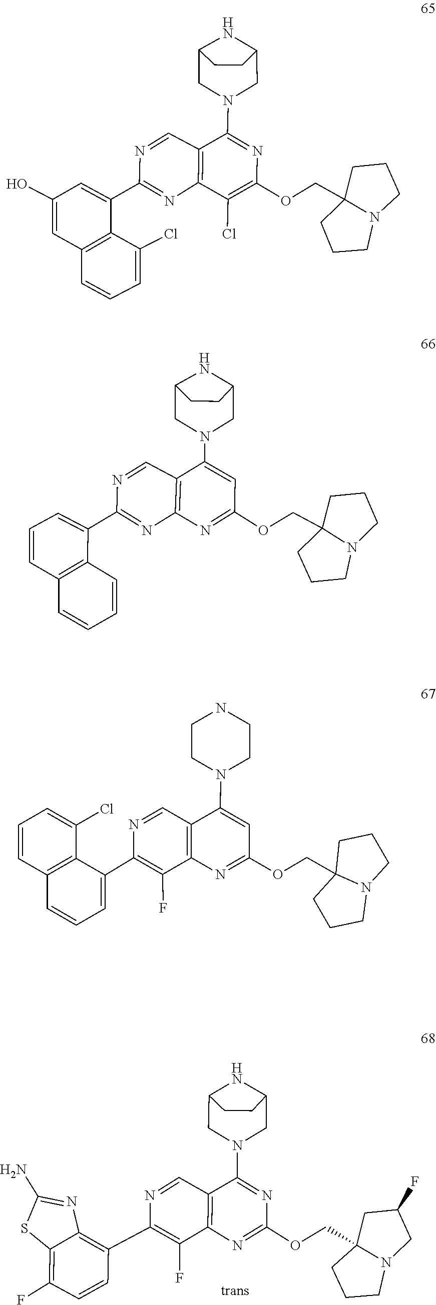

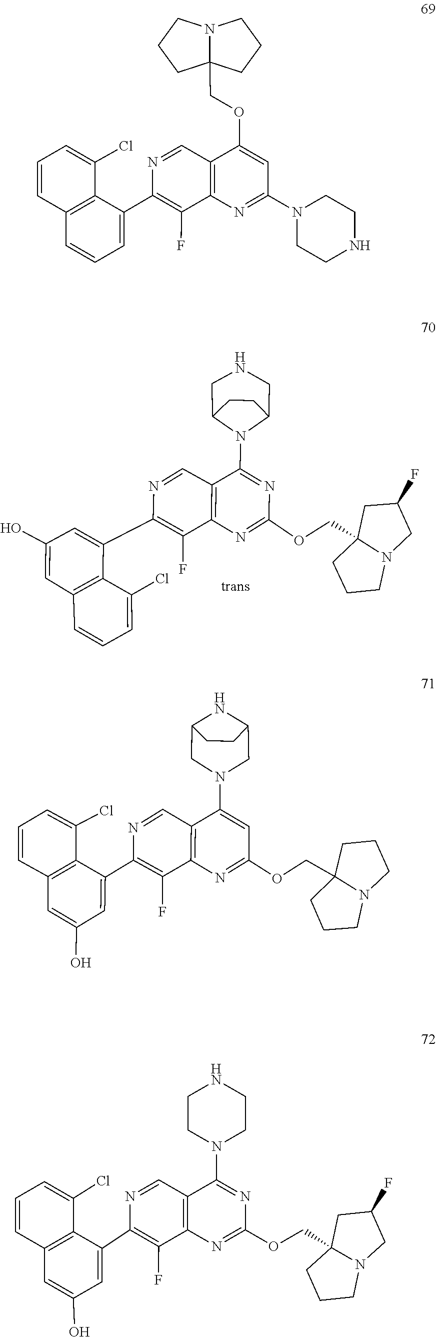 [SMILES: Fc1c(-c2cccc3cccc(Cl)c23)ncc2c(OCC34CCCN3CCC4)cc(N3CCNCC3)nc12 ; Oc1cc(-c2ncc3c(N4C5CCC4CNC5)nc(OC[C@@]45CCCN4C[C@H](F)C5)nc3c2F)c2c(Cl)cccc2c1 ; Oc1cc(-c2ncc3c(N4CC5CCC(C4)N5)cc(OCC45CCCN4CCC5)nc3c2F)c2c(Cl)cccc2c1 ; Oc1cc(-c2ncc3c(N4CCNCC4)nc(OC[C@@]45CCCN4C[C@H](F)C5)nc3c2F)c2c(Cl)cccc2c1]

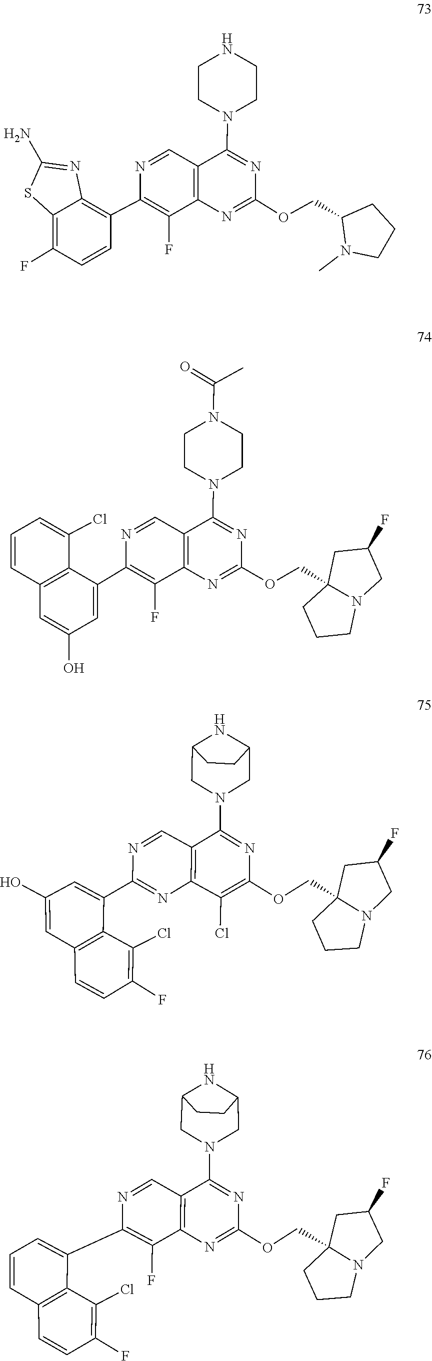 [SMILES: CN1CCC[C@H]1COc1nc(N2CCNCC2)c2cnc(-c3ccc(F)c4sc(N)nc34)c(F)c2n1 ; CC(=O)N1CCN(c2nc(OC[C@@]34CCCN3C[C@H](F)C4)nc3c(F)c(-c4cc(O)cc5cccc(Cl)c45)ncc23)CC1 ; Oc1cc(-c2ncc3c(N4CC5CCC(C4)N5)nc(OC[C@@]45CCCN4C[C@H](F)C5)c(Cl)c3n2)c2c(Cl)c(F)ccc2c1 ; Fc1ccc2cccc(-c3ncc4c(N5CC6CCC(C5)N6)nc(OC[C@@]56CCCN5C[C@H](F)C6)nc4c3F)c2c1Cl]

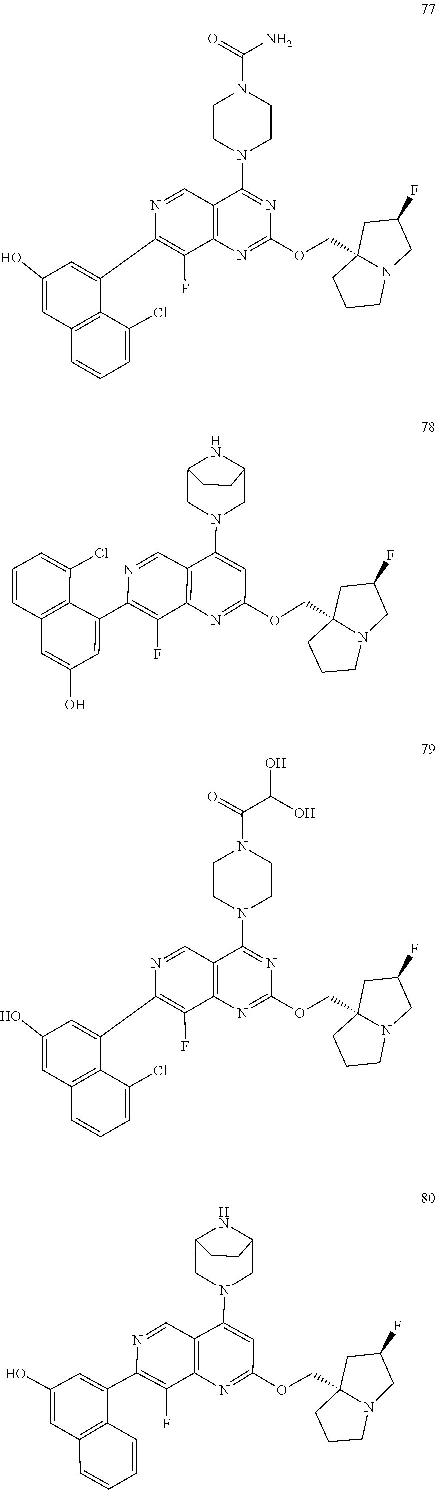 [SMILES: NC(=O)N1CCN(c2nc(OC[C@@]34CCCN3C[C@H](F)C4)nc3c(F)c(-c4cc(O)cc5cccc(Cl)c45)ncc23)CC1 ; Oc1cc(-c2ncc3c(N4CC5CCC(C4)N5)cc(OC[C@@]45CCCN4C[C@H](F)C5)nc3c2F)c2c(Cl)cccc2c1 ; O=C(C(O)O)N1CCN(c2nc(OC[C@@]34CCCN3C[C@H](F)C4)nc3c(F)c(-c4cc(O)cc5cccc(Cl)c45)ncc23)CC1 ; Oc1cc(-c2ncc3c(N4CC5CCC(C4)N5)cc(OC[C@@]45CCCN4C[C@H](F)C5)nc3c2F)c2ccccc2c1]

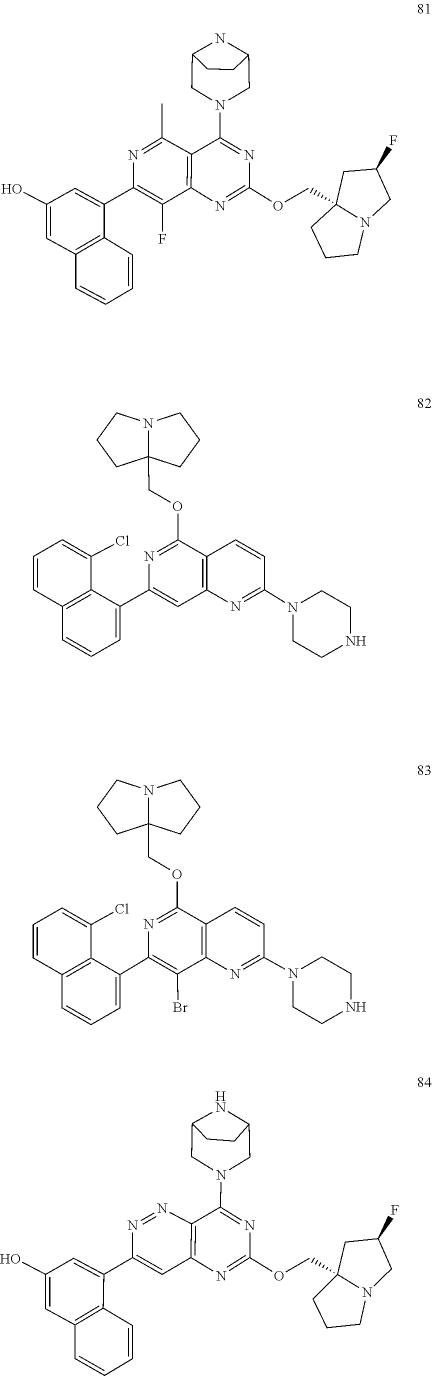

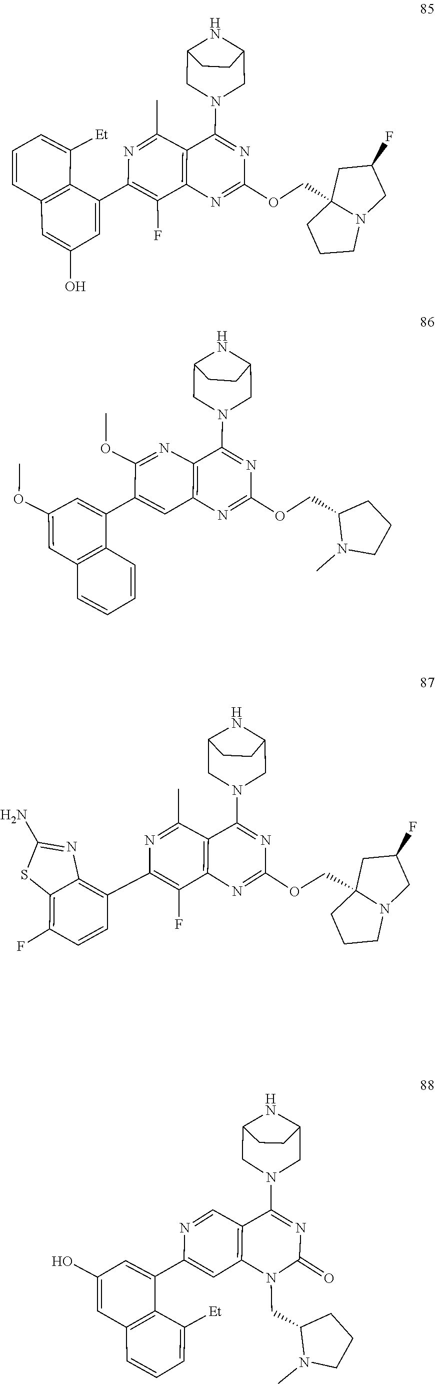 [SMILES: CCc1cccc2cc(O)cc(-c3nc(C)c4c(N5CC6CCC(C5)N6)nc(OC[C@@]56CCCN5C[C@H](F)C6)nc4c3F)c12 ; COc1cc(-c2cc3nc(OC[C@@H]4CCCN4C)nc(N4CC5CCC(C4)N5)c3nc2OC)c2ccccc2c1 ; Cc1nc(-c2ccc(F)c3sc(N)nc23)c(F)c2nc(OC[C@@]34CCCN3C[C@H](F)C4)nc(N3CC4CCC(C3)N4)c12 ; CCc1cccc2cc(O)cc(-c3cc4c(cn3)c(N3CC5CCC(C3)N5)nc(=O)n4C[C@@H]3CCCN3C)c12]

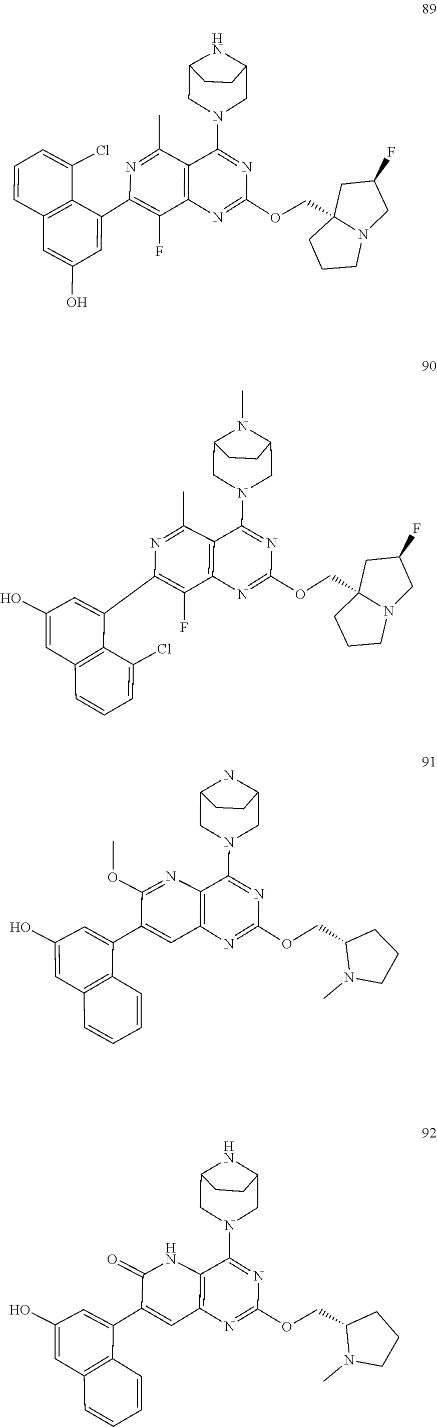

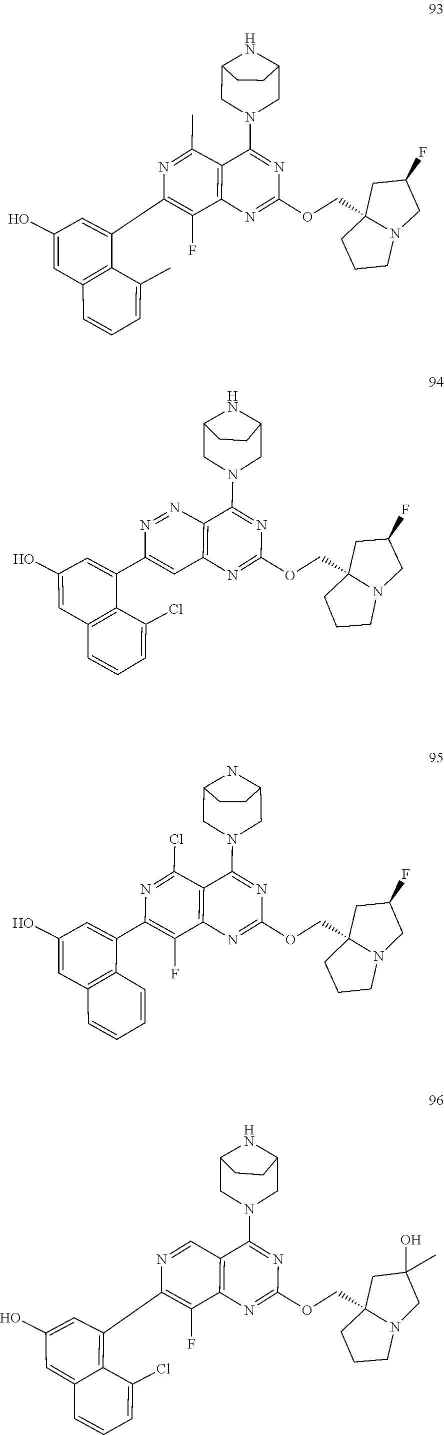

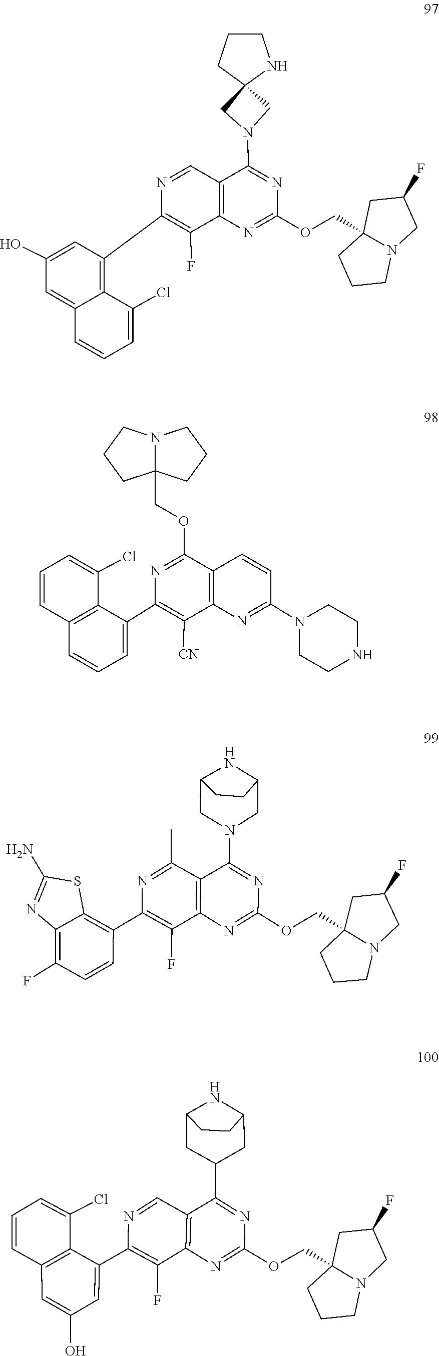

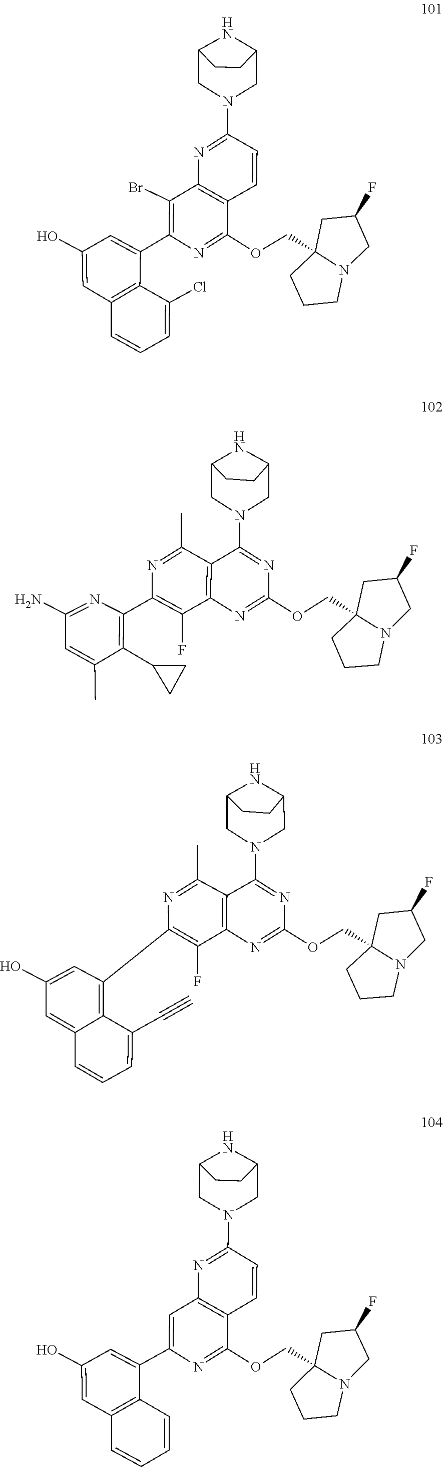 [SMILES: Oc1cc(-c2nc(OC[C@@]34CCCN3C[C@H](F)C4)c3ccc(N4CC5CCC(C4)N5)nc3c2Br)c2c(Cl)cccc2c1 ; Cc1cc(N)nc(-c2nc(C)c3c(N4CC5CCC(C4)N5)nc(OC[C@@]45CCCN4C[C@H](F)C5)nc3c2F)c1C1CC1 ; C#Cc1cccc2cc(O)cc(-c3nc(C)c4c(N5CC6CCC(C5)N6)nc(OC[C@@]56CCCN5C[C@H](F)C6)nc4c3F)c12 ; Oc1cc(-c2cc3nc(N4CC5CCC(C4)N5)ccc3c(OC[C@@]34CCCN3C[C@H](F)C4)n2)c2ccccc2c1]

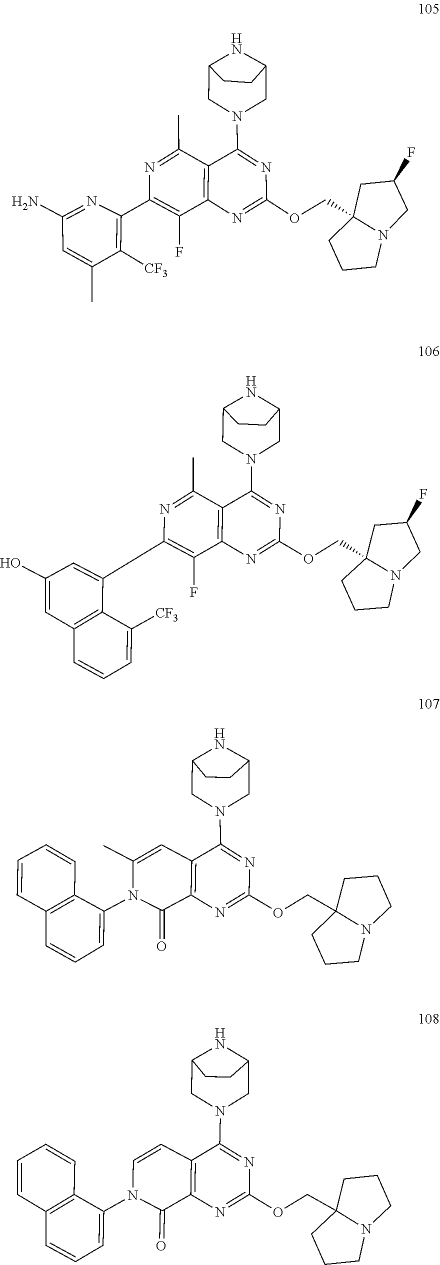 [SMILES: Cc1cc(N)nc(-c2nc(C)c3c(N4CC5CCC(C4)N5)nc(OC[C@@]45CCCN4C[C@H](F)C5)nc3c2F)c1C(F)(F)F ; Cc1nc(-c2cc(O)cc3cccc(C(F)(F)F)c23)c(F)c2nc(OC[C@@]34CCCN3C[C@H](F)C4)nc(N3CC4CCC(C3)N4)c12 ; Cc1cc2c(N3CC4CCC(C3)N4)nc(OCC34CCCN3CCC4)nc2c(=O)n1-c1cccc2ccccc12 ; O=c1c2nc(OCC34CCCN3CCC4)nc(N3CC4CCC(C3)N4)c2ccn1-c1cccc2ccccc12]

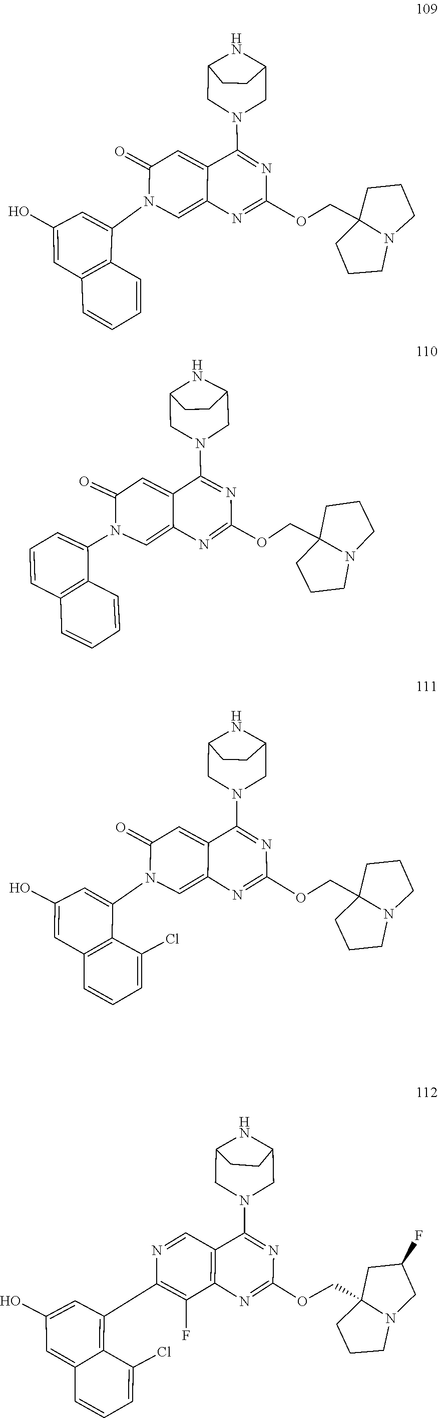 [SMILES: O=c1cc2c(N3CC4CCC(C3)N4)nc(OCC34CCCN3CCC4)nc2cn1-c1cc(O)cc2ccccc12 ; O=c1cc2c(N3CC4CCC(C3)N4)nc(OCC34CCCN3CCC4)nc2cn1-c1cccc2ccccc12 ; O=c1cc2c(N3CC4CCC(C3)N4)nc(OCC34CCCN3CCC4)nc2cn1-c1cc(O)cc2cccc(Cl)c12 ; Oc1cc(-c2ncc3c(N4CC5CCC(C4)N5)nc(OC[C@@]45CCCN4C[C@H](F)C5)nc3c2F)c2c(Cl)cccc2c1]

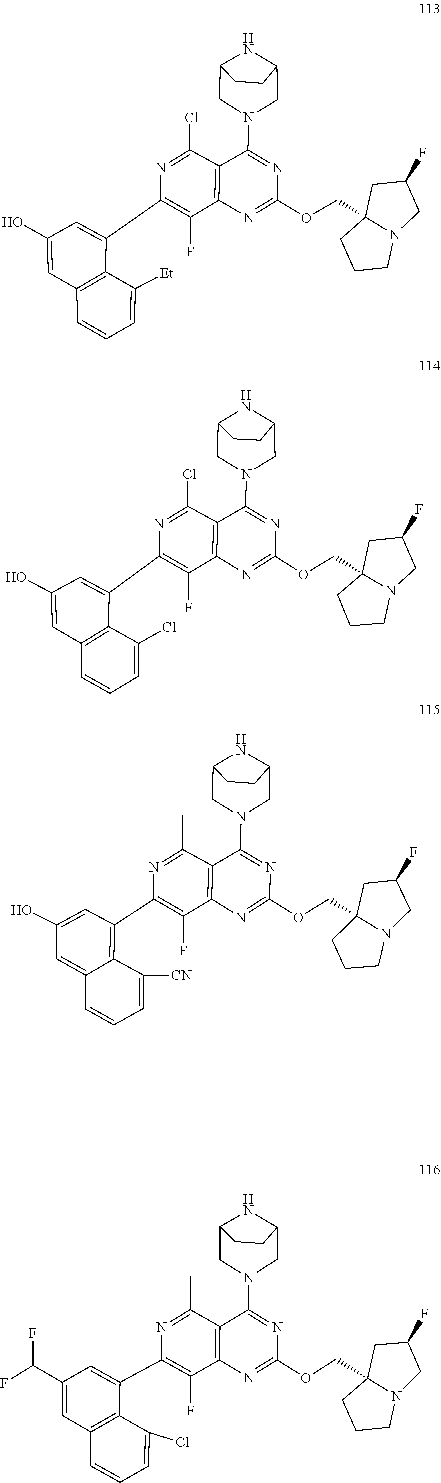 [SMILES: CCc1cccc2cc(O)cc(-c3nc(Cl)c4c(N5CC6CCC(C5)N6)nc(OC[C@@]56CCCN5C[C@H](F)C6)nc4c3F)c12 ; Oc1cc(-c2nc(Cl)c3c(N4CC5CCC(C4)N5)nc(OC[C@@]45CCCN4C[C@H](F)C5)nc3c2F)c2c(Cl)cccc2c1 ; Cc1nc(-c2cc(O)cc3cccc(C#N)c23)c(F)c2nc(OC[C@@]34CCCN3C[C@H](F)C4)nc(N3CC4CCC(C3)N4)c12 ; Cc1nc(-c2cc(C(F)F)cc3cccc(Cl)c23)c(F)c2nc(OC[C@@]34CCCN3C[C@H](F)C4)nc(N3CC4CCC(C3)N4)c12]

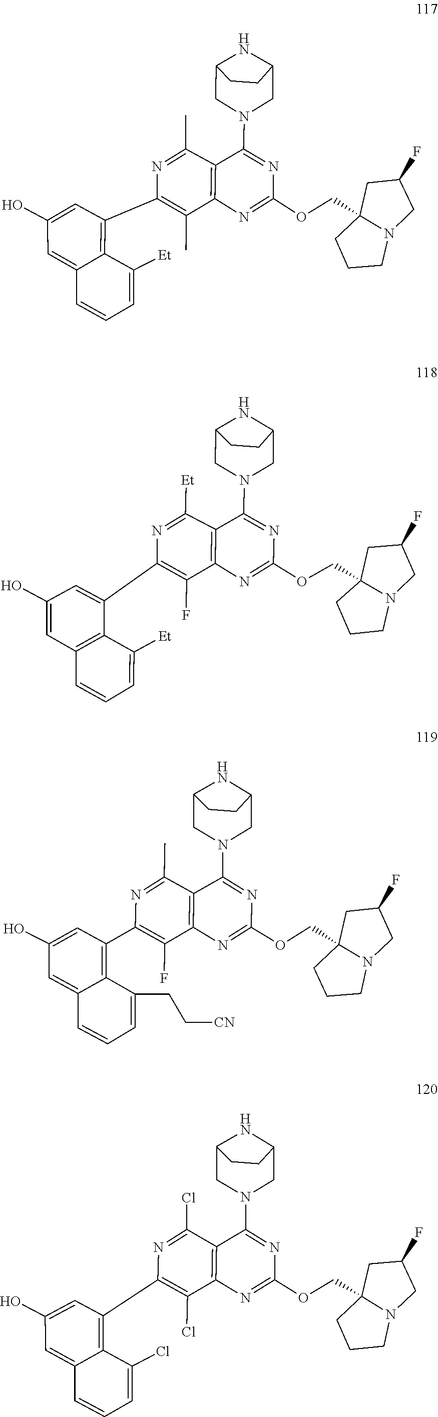 [SMILES: CCc1cccc2cc(O)cc(-c3nc(C)c4c(N5CC6CCC(C5)N6)nc(OC[C@@]56CCCN5C[C@H](F)C6)nc4c3C)c12 ; CCc1cccc2cc(O)cc(-c3nc(CC)c4c(N5CC6CCC(C5)N6)nc(OC[C@@]56CCCN5C[C@H](F)C6)nc4c3F)c12 ; Cc1nc(-c2cc(O)cc3cccc(CCC#N)c23)c(F)c2nc(OC[C@@]34CCCN3C[C@H](F)C4)nc(N3CC4CCC(C3)N4)c12 ; Oc1cc(-c2nc(Cl)c3c(N4CC5CCC(C4)N5)nc(OC[C@@]45CCCN4C[C@H](F)C5)nc3c2Cl)c2c(Cl)cccc2c1]

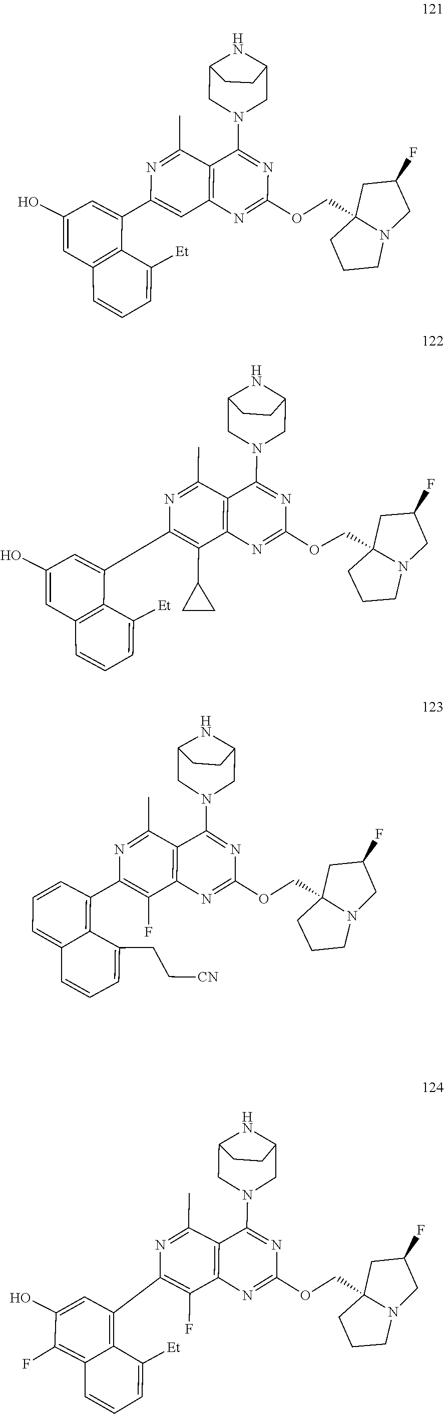 [SMILES: CCc1cccc2cc(O)cc(-c3cc4nc(OC[C@@]56CCCN5C[C@H](F)C6)nc(N5CC6CCC(C5)N6)c4c(C)n3)c12 ; CCc1cccc2cc(O)cc(-c3nc(C)c4c(N5CC6CCC(C5)N6)nc(OC[C@@]56CCCN5C[C@H](F)C6)nc4c3C3CC3)c12 ; Cc1nc(-c2cccc3cccc(CCC#N)c23)c(F)c2nc(OC[C@@]34CCCN3C[C@H](F)C4)nc(N3CC4CCC(C3)N4)c12 ; CCc1cccc2c(F)c(O)cc(-c3nc(C)c4c(N5CC6CCC(C5)N6)nc(OC[C@@]56CCCN5C[C@H](F)C6)nc4c3F)c12]

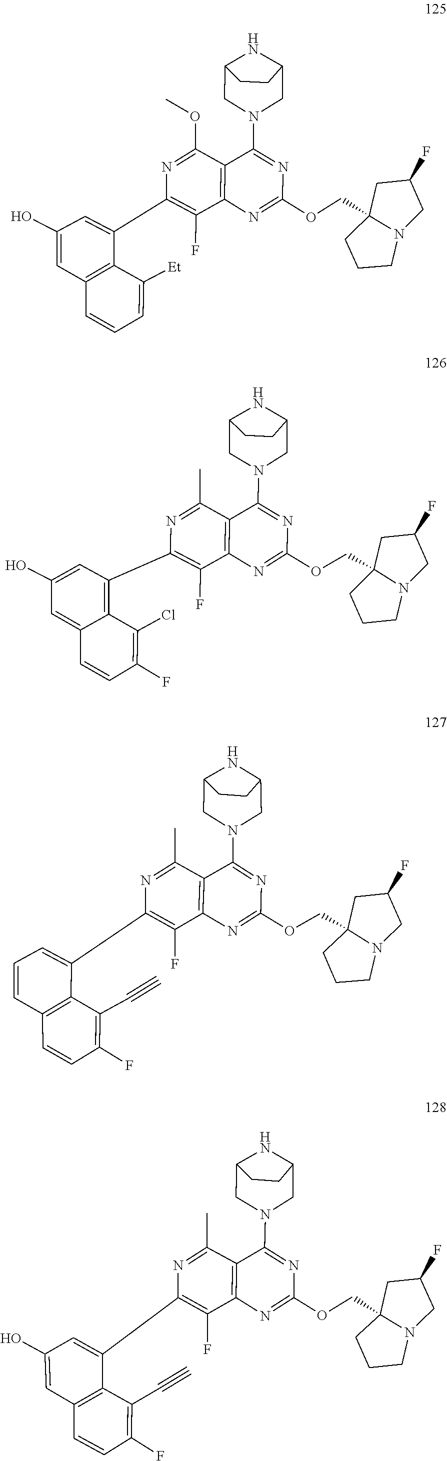 [SMILES: CCc1cccc2cc(O)cc(-c3nc(OC)c4c(N5CC6CCC(C5)N6)nc(OC[C@@]56CCCN5C[C@H](F)C6)nc4c3F)c12 ; Cc1nc(-c2cc(O)cc3ccc(F)c(Cl)c23)c(F)c2nc(OC[C@@]34CCCN3C[C@H](F)C4)nc(N3CC4CCC(C3)N4)c12 ; C#Cc1c(F)ccc2cccc(-c3nc(C)c4c(N5CC6CCC(C5)N6)nc(OC[C@@]56CCCN5C[C@H](F)C6)nc4c3F)c12 ; C#Cc1c(F)ccc2cc(O)cc(-c3nc(C)c4c(N5CC6CCC(C5)N6)nc(OC[C@@]56CCCN5C[C@H](F)C6)nc4c3F)c12]

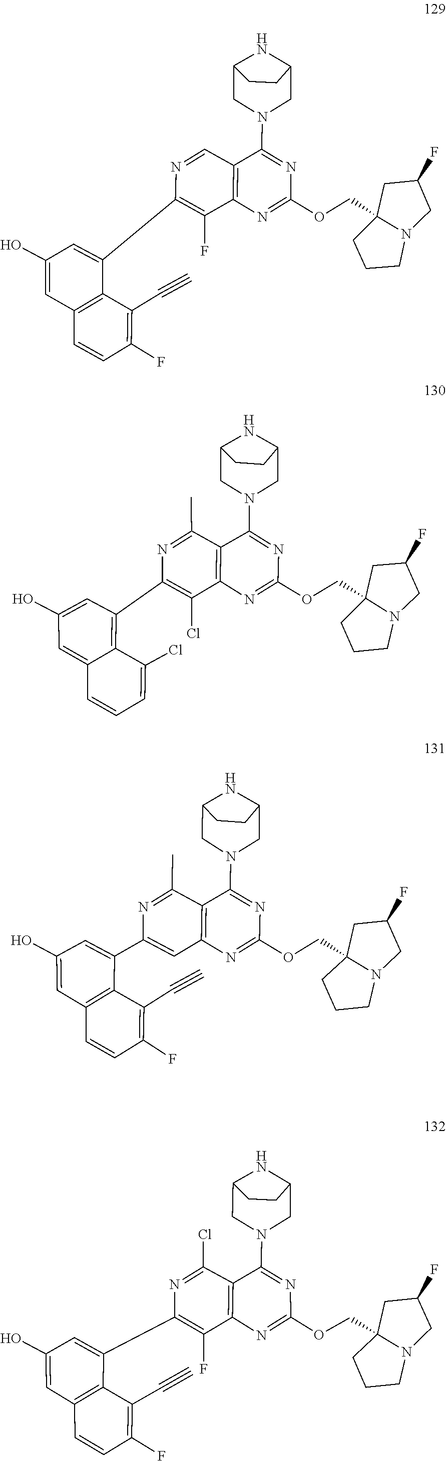 [SMILES: C#Cc1c(F)ccc2cc(O)cc(-c3ncc4c(N5CC6CCC(C5)N6)nc(OC[C@@]56CCCN5C[C@H](F)C6)nc4c3F)c12 ; Cc1nc(-c2cc(O)cc3cccc(Cl)c23)c(Cl)c2nc(OC[C@@]34CCCN3C[C@H](F)C4)nc(N3CC4CCC(C3)N4)c12 ; C#Cc1c(F)ccc2cc(O)cc(-c3cc4nc(OC[C@@]56CCCN5C[C@H](F)C6)nc(N5CC6CCC(C5)N6)c4c(C)n3)c12 ; C#Cc1c(F)ccc2cc(O)cc(-c3nc(Cl)c4c(N5CC6CCC(C5)N6)nc(OC[C@@]56CCCN5C[C@H](F)C6)nc4c3F)c12]

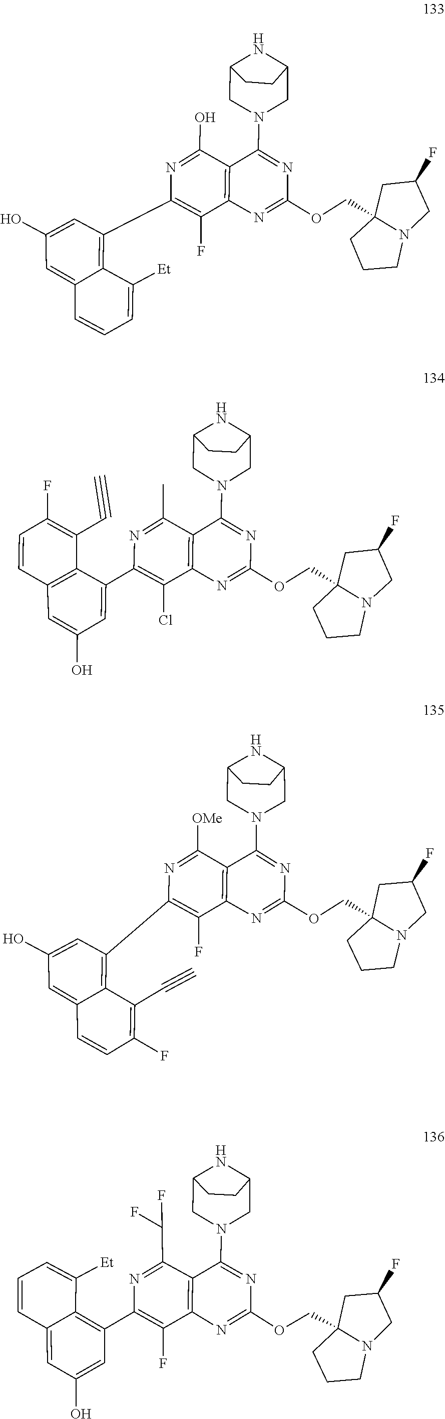 [SMILES: CCc1cccc2cc(O)cc(-c3nc(O)c4c(N5CC6CCC(C5)N6)nc(OC[C@@]56CCCN5C[C@H](F)C6)nc4c3F)c12 ; C#Cc1c(F)ccc2cc(O)cc(-c3nc(C)c4c(N5CC6CCC(C5)N6)nc(OC[C@@]56CCCN5C[C@H](F)C6)nc4c3Cl)c12 ; C#Cc1c(F)ccc2cc(O)cc(-c3nc(OC)c4c(N5CC6CCC(C5)N6)nc(OC[C@@]56CCCN5C[C@H](F)C6)nc4c3F)c12 ; CCc1cccc2cc(O)cc(-c3nc(C(F)F)c4c(N5CC6CCC(C5)N6)nc(OC[C@@]56CCCN5C[C@H](F)C6)nc4c3F)c12]

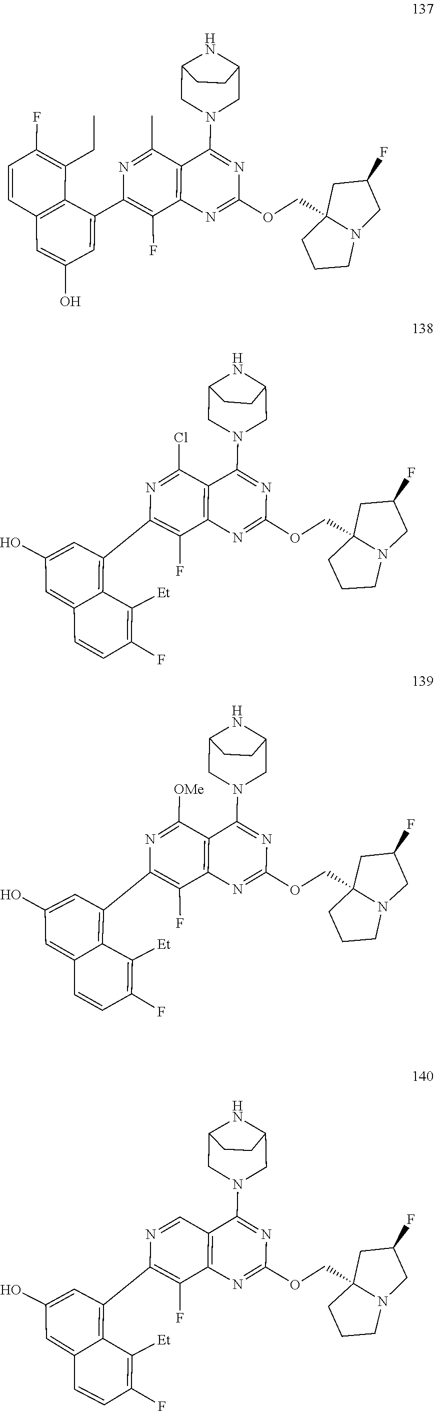 [SMILES: CCc1c(F)ccc2cc(O)cc(-c3nc(C)c4c(N5CC6CCC(C5)N6)nc(OC[C@@]56CCCN5C[C@H](F)C6)nc4c3F)c12 ; CCc1c(F)ccc2cc(O)cc(-c3nc(Cl)c4c(N5CC6CCC(C5)N6)nc(OC[C@@]56CCCN5C[C@H](F)C6)nc4c3F)c12 ; CCc1c(F)ccc2cc(O)cc(-c3nc(OC)c4c(N5CC6CCC(C5)N6)nc(OC[C@@]56CCCN5C[C@H](F)C6)nc4c3F)c12 ; CCc1c(F)ccc2cc(O)cc(-c3ncc4c(N5CC6CCC(C5)N6)nc(OC[C@@]56CCCN5C[C@H](F)C6)nc4c3F)c12]

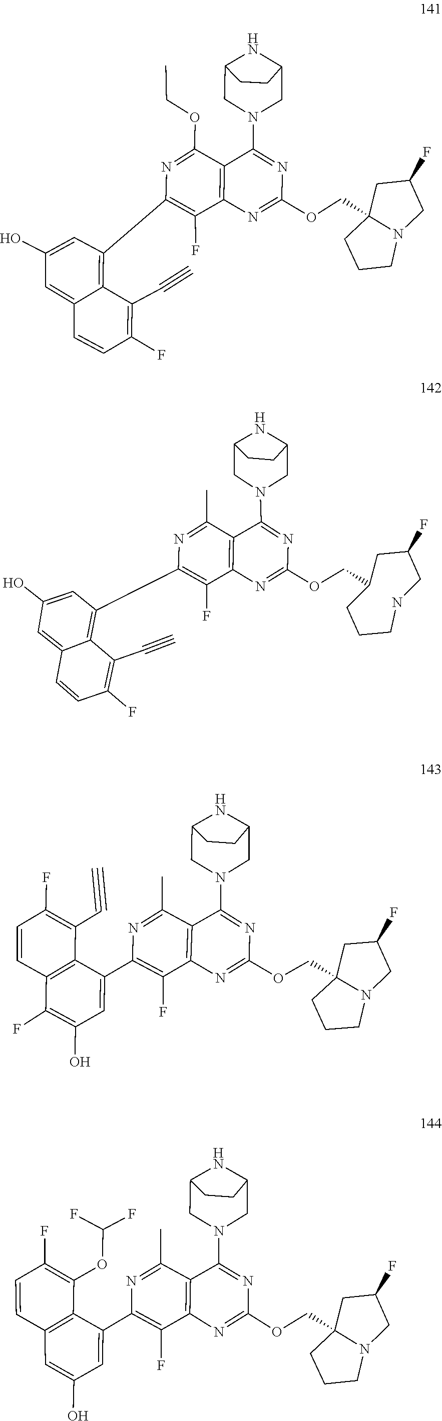

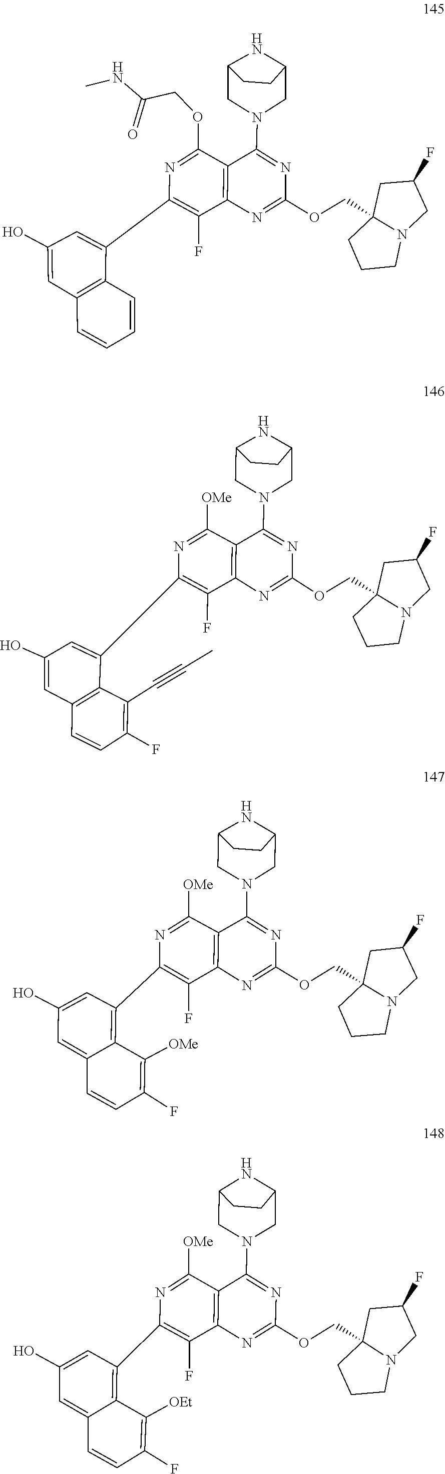 [SMILES: CNC(=O)COc1nc(-c2cc(O)cc3ccccc23)c(F)c2nc(OC[C@@]34CCCN3C[C@H](F)C4)nc(N3CC4CCC(C3)N4)c12 ; CC#Cc1c(F)ccc2cc(O)cc(-c3nc(OC)c4c(N5CC6CCC(C5)N6)nc(OC[C@@]56CCCN5C[C@H](F)C6)nc4c3F)c12 ; COc1c(F)ccc2cc(O)cc(-c3nc(OC)c4c(N5CC6CCC(C5)N6)nc(OC[C@@]56CCCN5C[C@H](F)C6)nc4c3F)c12 ; CCOc1c(F)ccc2cc(O)cc(-c3nc(OC)c4c(N5CC6CCC(C5)N6)nc(OC[C@@]56CCCN5C[C@H](F)C6)nc4c3F)c12]

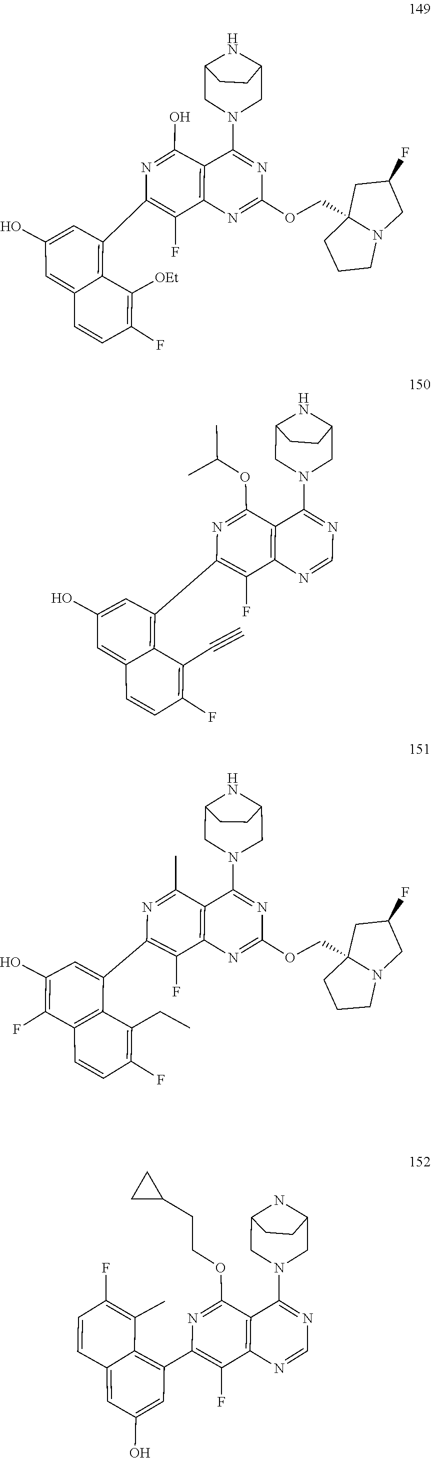

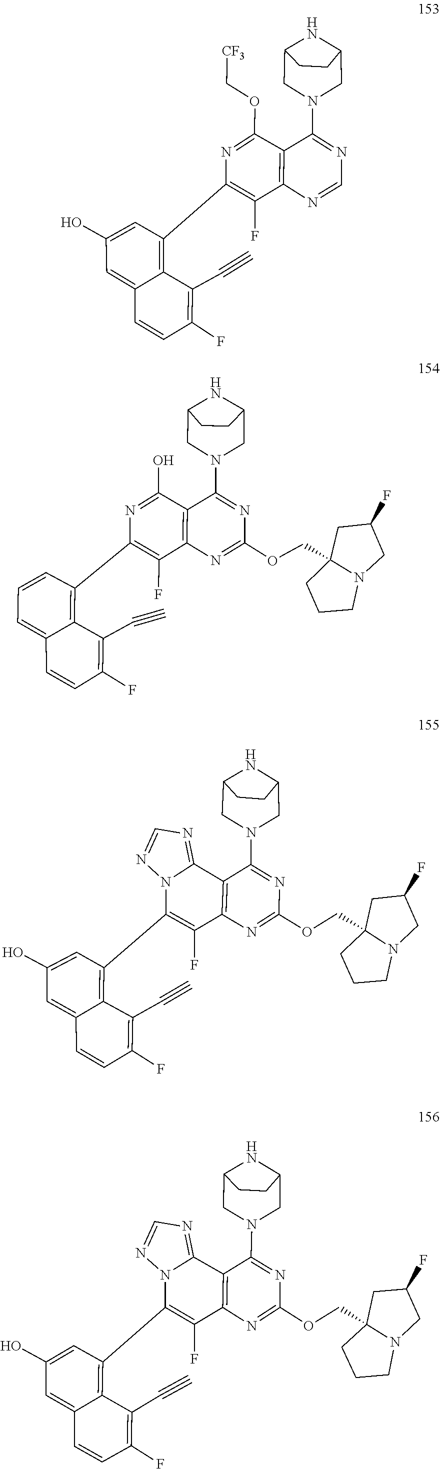 [SMILES: C#Cc1c(F)ccc2cc(O)cc(-c3nc(OCC(F)(F)F)c4c(N5CC6CCC(C5)N6)ncnc4c3F)c12 ; C#Cc1c(F)ccc2cccc(-c3nc(O)c4c(N5CC6CCC(C5)N6)nc(OC[C@@]56CCCN5C[C@H](F)C6)nc4c3F)c12 ; C#Cc1c(F)ccc2cc(O)cc(-c3c(F)c4nc(OC[C@@]56CCCN5C[C@H](F)C6)nc(N5CC6CCC(C5)N6)c4c4ncnn34)c12 ; C#Cc1c(F)ccc2cc(O)cc(-c3c(F)c4nc(OC[C@@]56CCCN5C[C@H](F)C6)nc(N5CC6CCC(C5)N6)c4c4ncnn34)c12]

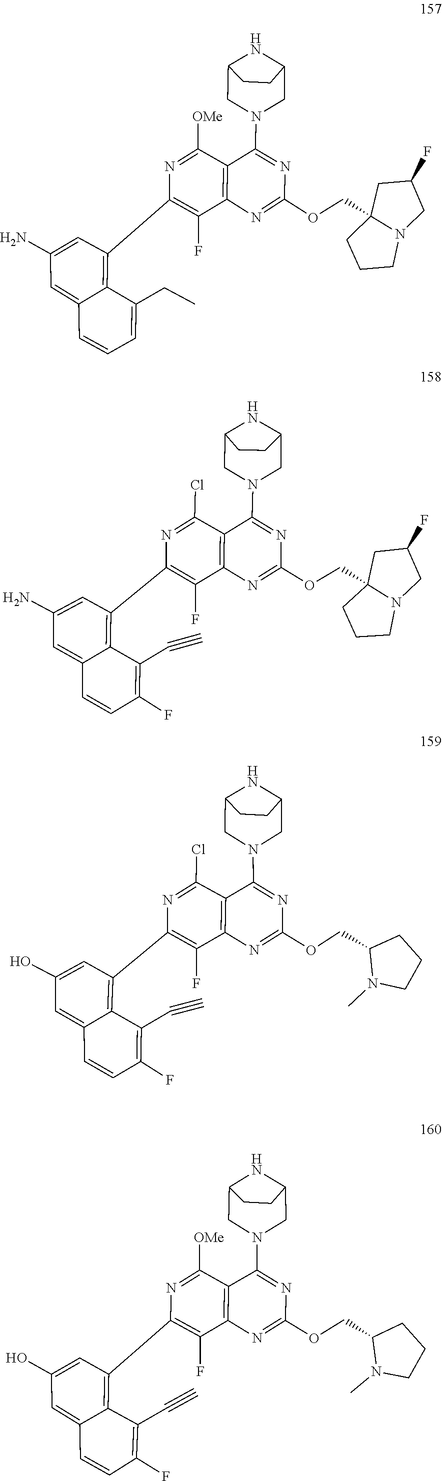 [SMILES: CCc1cccc2cc(N)cc(-c3nc(OC)c4c(N5CC6CCC(C5)N6)nc(OC[C@@]56CCCN5C[C@H](F)C6)nc4c3F)c12 ; C#Cc1c(F)ccc2cc(N)cc(-c3nc(Cl)c4c(N5CC6CCC(C5)N6)nc(OC[C@@]56CCCN5C[C@H](F)C6)nc4c3F)c12 ; C#Cc1c(F)ccc2cc(O)cc(-c3nc(Cl)c4c(N5CC6CCC(C5)N6)nc(OC[C@@H]5CCCN5C)nc4c3F)c12 ; C#Cc1c(F)ccc2cc(O)cc(-c3nc(OC)c4c(N5CC6CCC(C5)N6)nc(OC[C@@H]5CCCN5C)nc4c3F)c12]

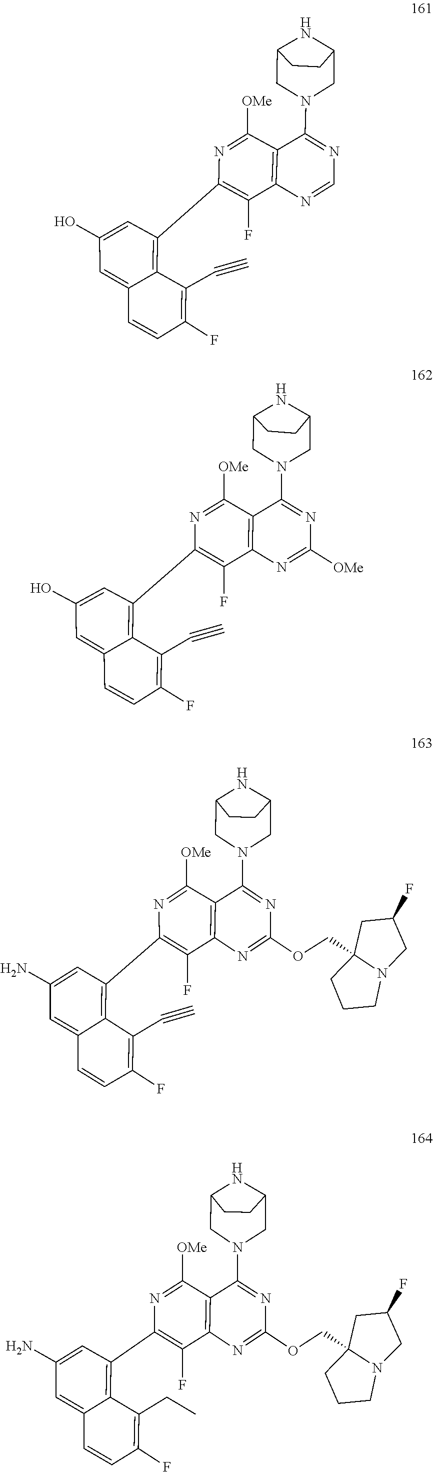 [SMILES: C#Cc1c(F)ccc2cc(O)cc(-c3nc(OC)c4c(N5CC6CCC(C5)N6)ncnc4c3F)c12 ; C#Cc1c(F)ccc2cc(O)cc(-c3nc(OC)c4c(N5CC6CCC(C5)N6)nc(OC)nc4c3F)c12 ; C#Cc1c(F)ccc2cc(N)cc(-c3nc(OC)c4c(N5CC6CCC(C5)N6)nc(OC[C@@]56CCCN5C[C@H](F)C6)nc4c3F)c12 ; CCc1c(F)ccc2cc(N)cc(-c3nc(OC)c4c(N5CC6CCC(C5)N6)nc(OC[C@@]56CCCN5C[C@H](F)C6)nc4c3F)c12]

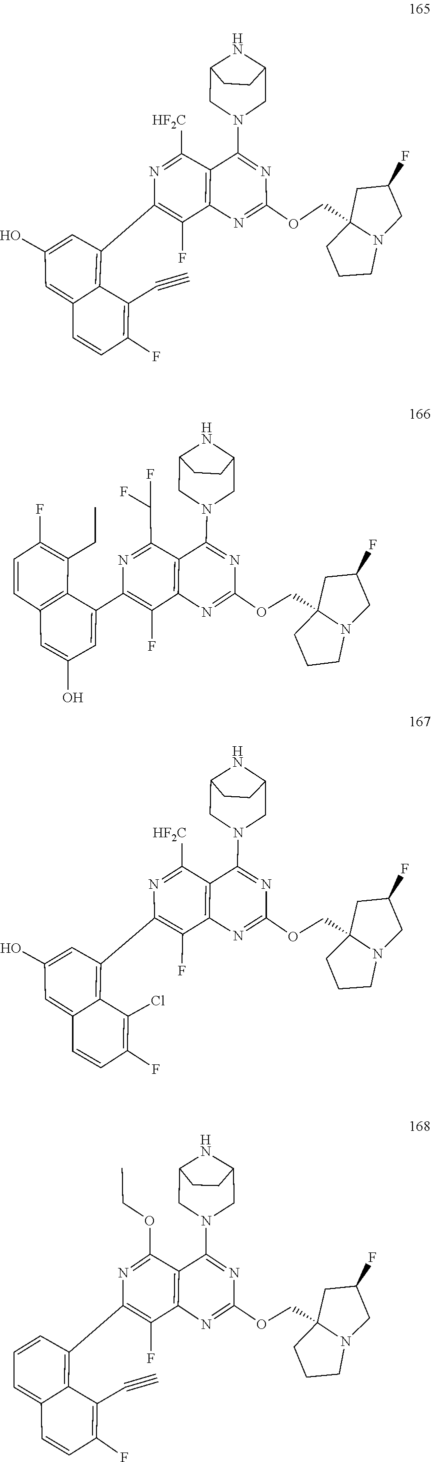

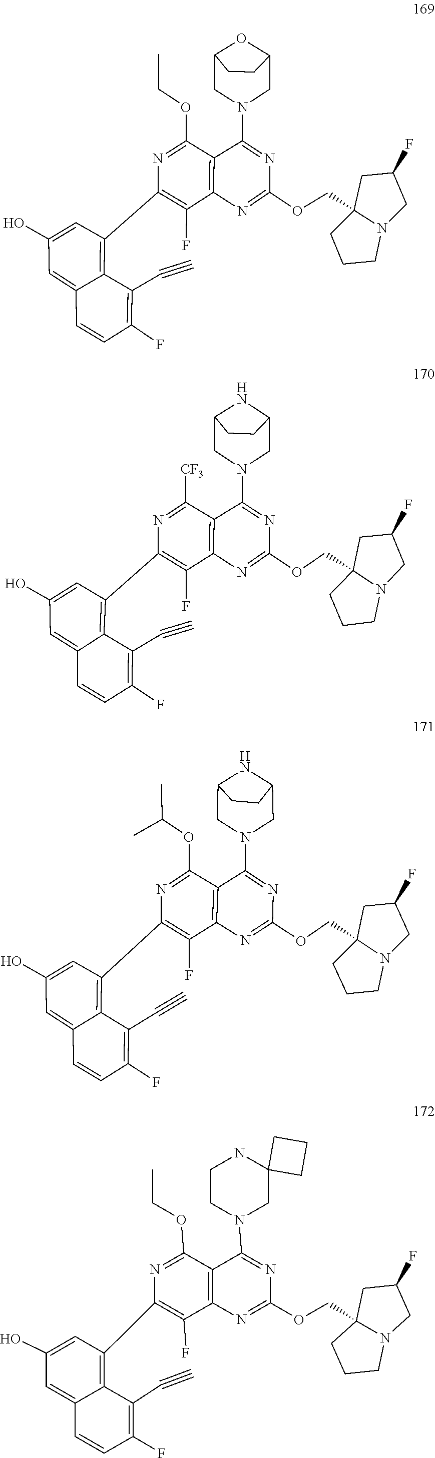

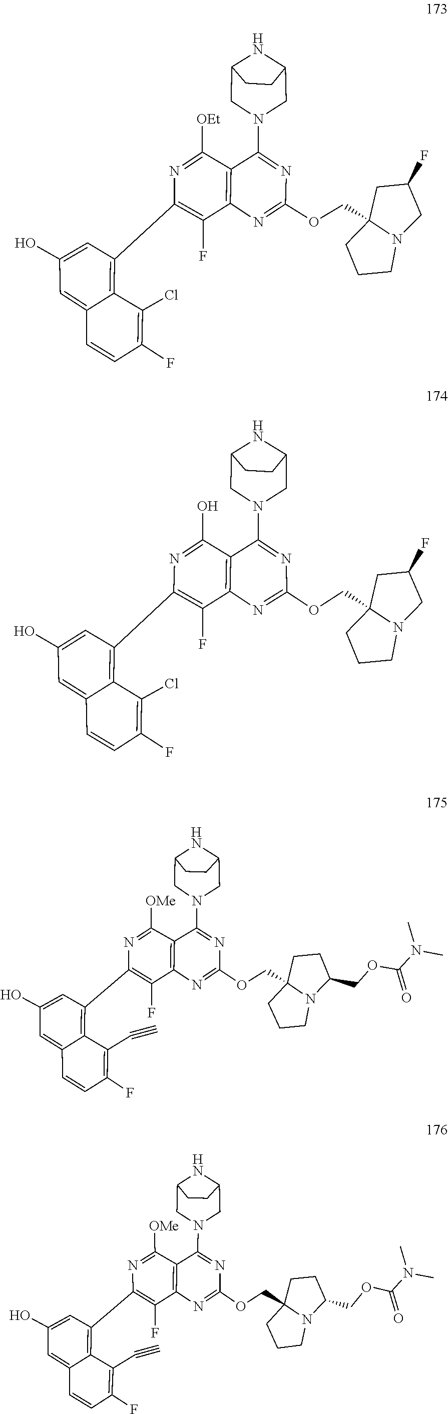 [SMILES: CCOc1nc(-c2cc(O)cc3ccc(F)c(Cl)c23)c(F)c2nc(OC[C@@]34CCCN3C[C@H](F)C4)nc(N3CC4CCC(C3)N4)c12 ; Oc1cc(-c2nc(O)c3c(N4CC5CCC(C4)N5)nc(OC[C@@]45CCCN4C[C@H](F)C5)nc3c2F)c2c(Cl)c(F)ccc2c1 ; C#Cc1c(F)ccc2cc(O)cc(-c3nc(OC)c4c(N5CC6CCC(C5)N6)nc(OC[C@@]56CCCN5[C@H](COC(=O)N(C)C)CC6)nc4c3F)c12 ; C#Cc1c(F)ccc2cc(O)cc(-c3nc(OC)c4c(N5CC6CCC(C5)N6)nc(OC[C@]56CCCN5[C@@H](COC(=O)N(C)C)CC6)nc4c3F)c12]

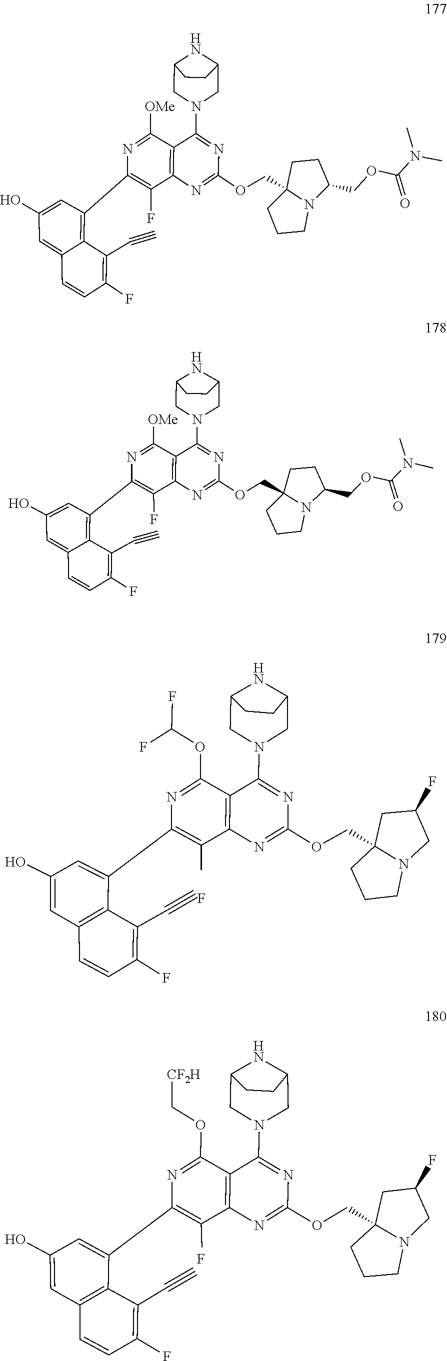

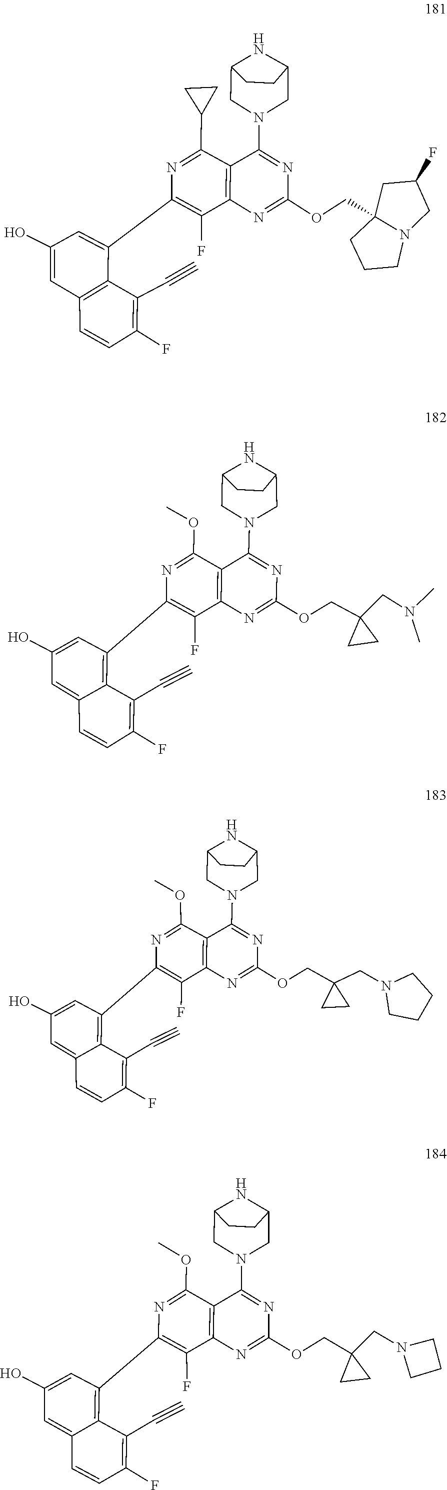

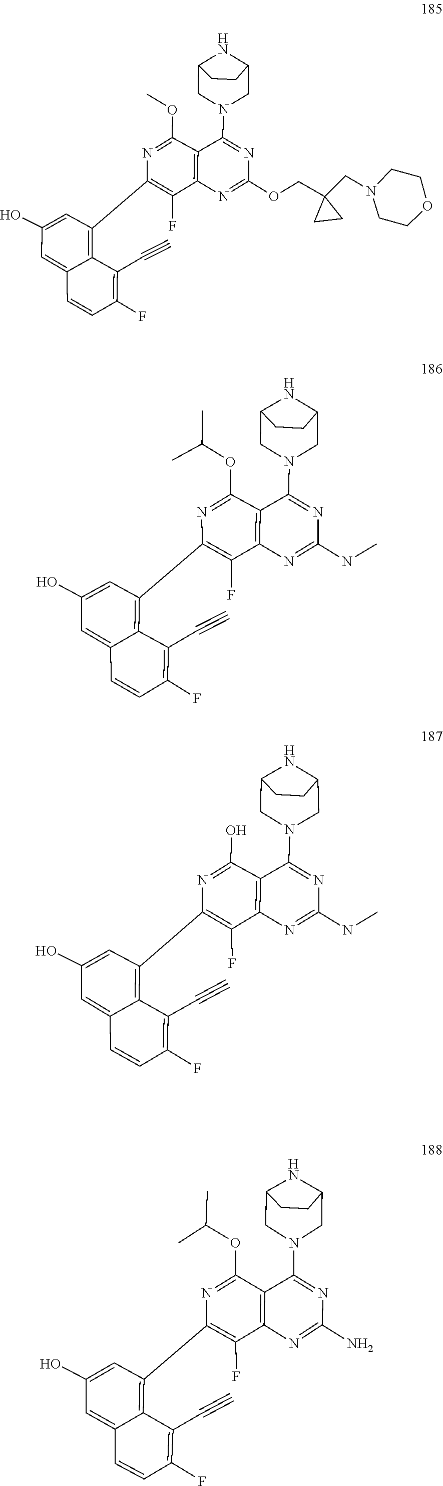

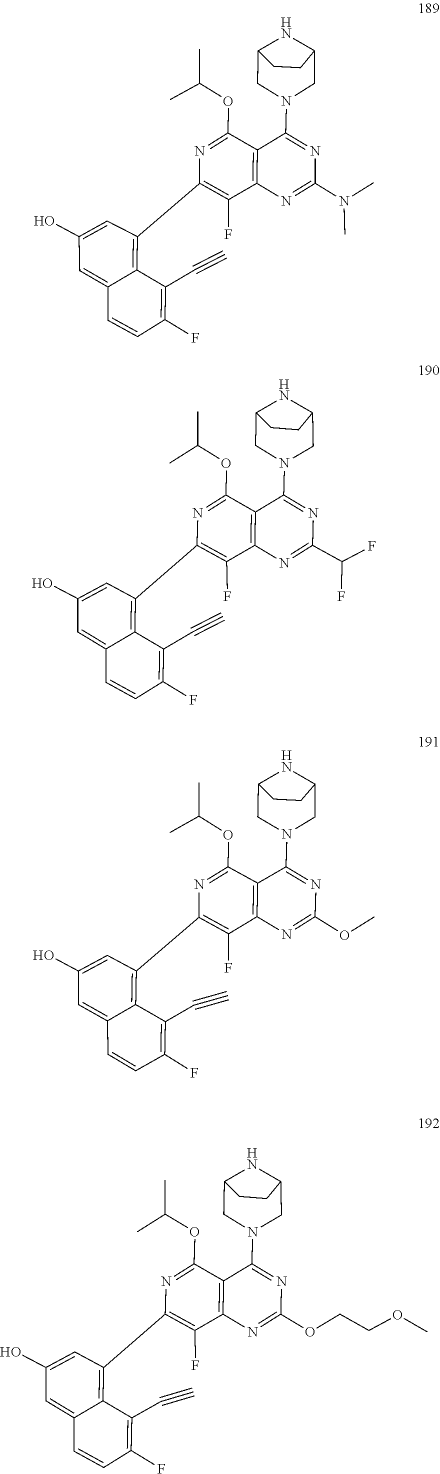 [SMILES: C#Cc1c(F)ccc2cc(O)cc(-c3nc(OC(C)C)c4c(N5CC6CCC(C5)N6)nc(N(C)C)nc4c3F)c12 ; C#Cc1c(F)ccc2cc(O)cc(-c3nc(OC(C)C)c4c(N5CC6CCC(C5)N6)nc(C(F)F)nc4c3F)c12 ; C#Cc1c(F)ccc2cc(O)cc(-c3nc(OC(C)C)c4c(N5CC6CCC(C5)N6)nc(OC)nc4c3F)c12 ; C#Cc1c(F)ccc2cc(O)cc(-c3nc(OC(C)C)c4c(N5CC6CCC(C5)N6)nc(OCCOC)nc4c3F)c12]

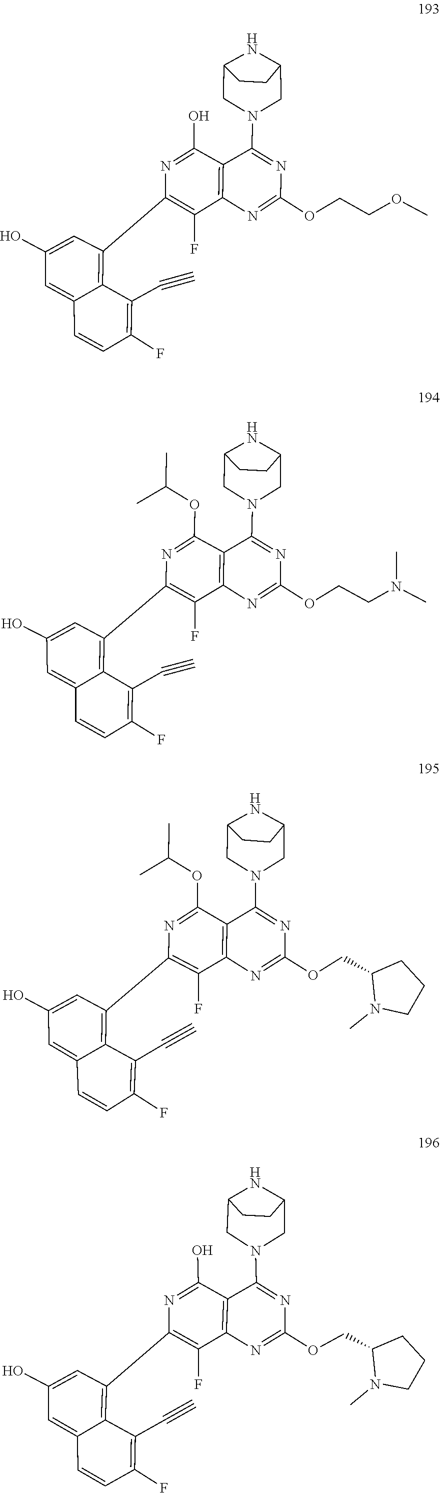 [SMILES: C#Cc1c(F)ccc2cc(O)cc(-c3nc(O)c4c(N5CC6CCC(C5)N6)nc(OCCOC)nc4c3F)c12 ; C#Cc1c(F)ccc2cc(O)cc(-c3nc(OC(C)C)c4c(N5CC6CCC(C5)N6)nc(OCCN(C)C)nc4c3F)c12 ; C#Cc1c(F)ccc2cc(O)cc(-c3nc(OC(C)C)c4c(N5CC6CCC(C5)N6)nc(OC[C@@H]5CCCN5C)nc4c3F)c12 ; C#Cc1c(F)ccc2cc(O)cc(-c3nc(O)c4c(N5CC6CCC(C5)N6)nc(OC[C@@H]5CCCN5C)nc4c3F)c12]

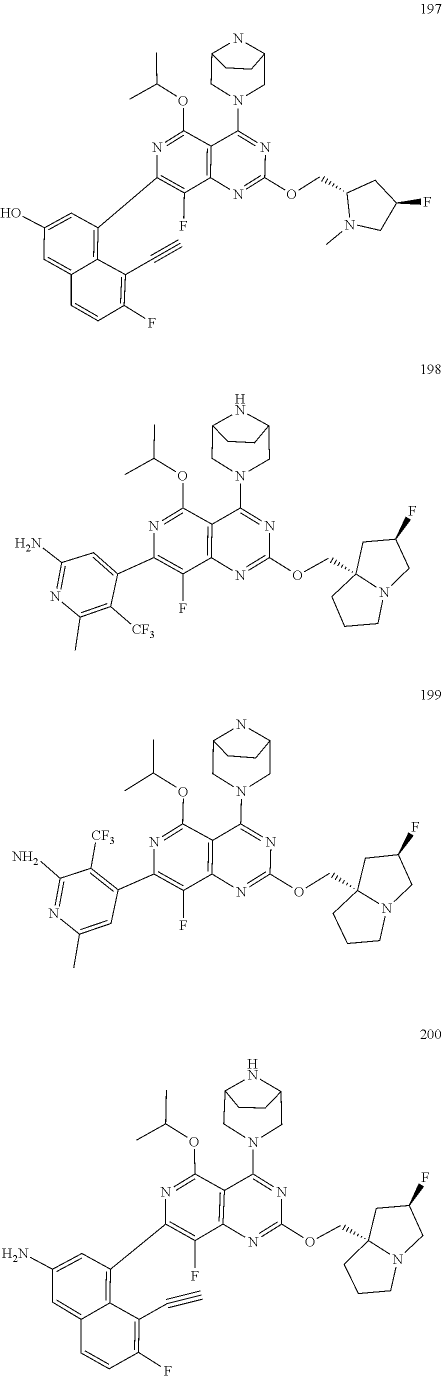

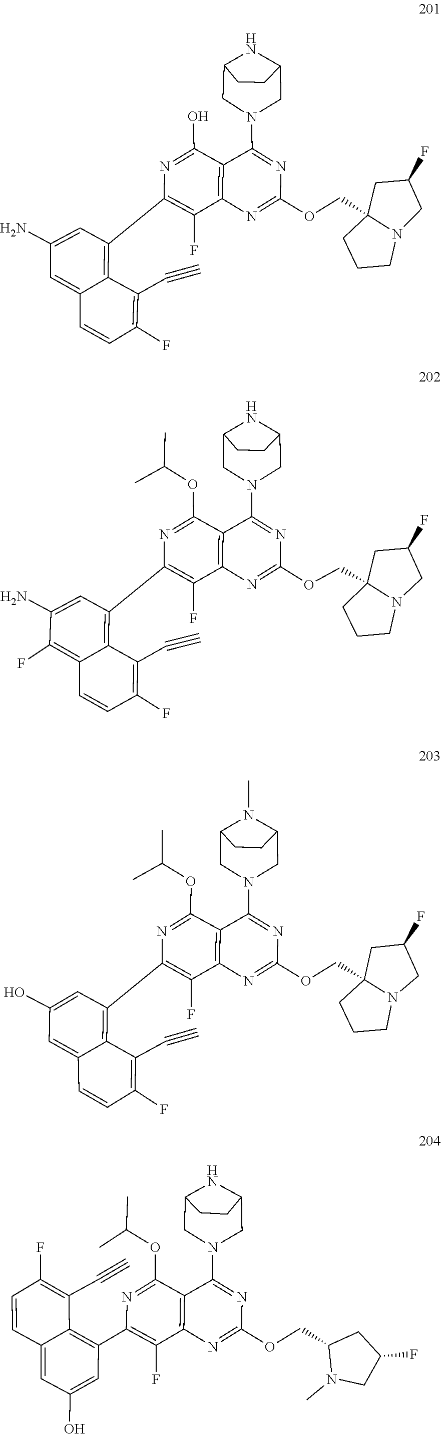 [SMILES: C#Cc1c(F)ccc2cc(N)cc(-c3nc(O)c4c(N5CC6CCC(C5)N6)nc(OC[C@@]56CCCN5C[C@H](F)C6)nc4c3F)c12 ; C#Cc1c(F)ccc2c(F)c(N)cc(-c3nc(OC(C)C)c4c(N5CC6CCC(C5)N6)nc(OC[C@@]56CCCN5C[C@H](F)C6)nc4c3F)c12 ; C#Cc1c(F)ccc2cc(O)cc(-c3nc(OC(C)C)c4c(N5CC6CCC(C5)N6C)nc(OC[C@@]56CCCN5C[C@H](F)C6)nc4c3F)c12 ; C#Cc1c(F)ccc2cc(O)cc(-c3nc(OC(C)C)c4c(N5CC6CCC(C5)N6)nc(OC[C@@H]5C[C@H](F)CN5C)nc4c3F)c12]

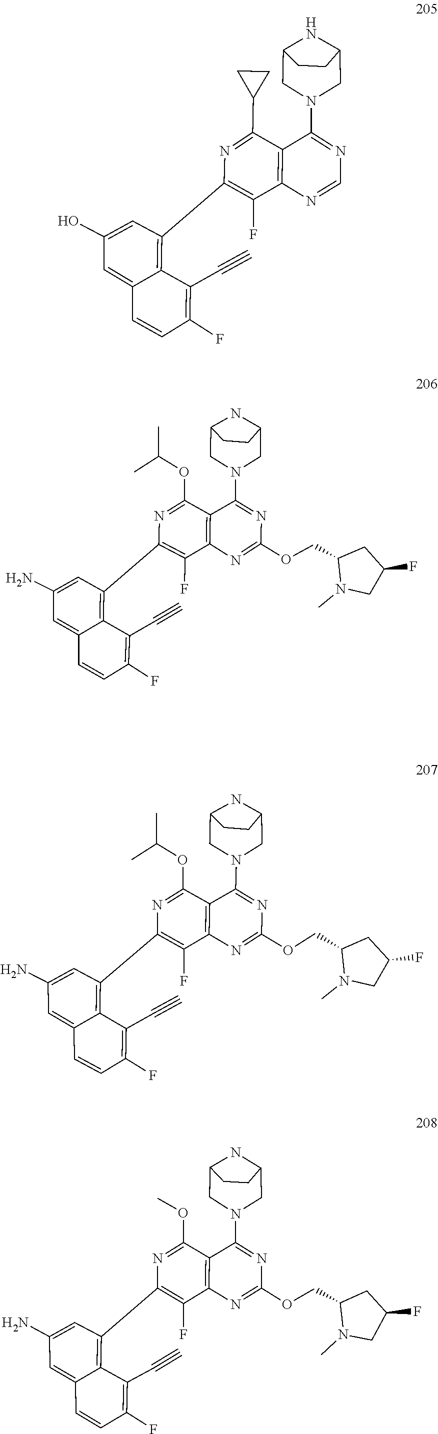

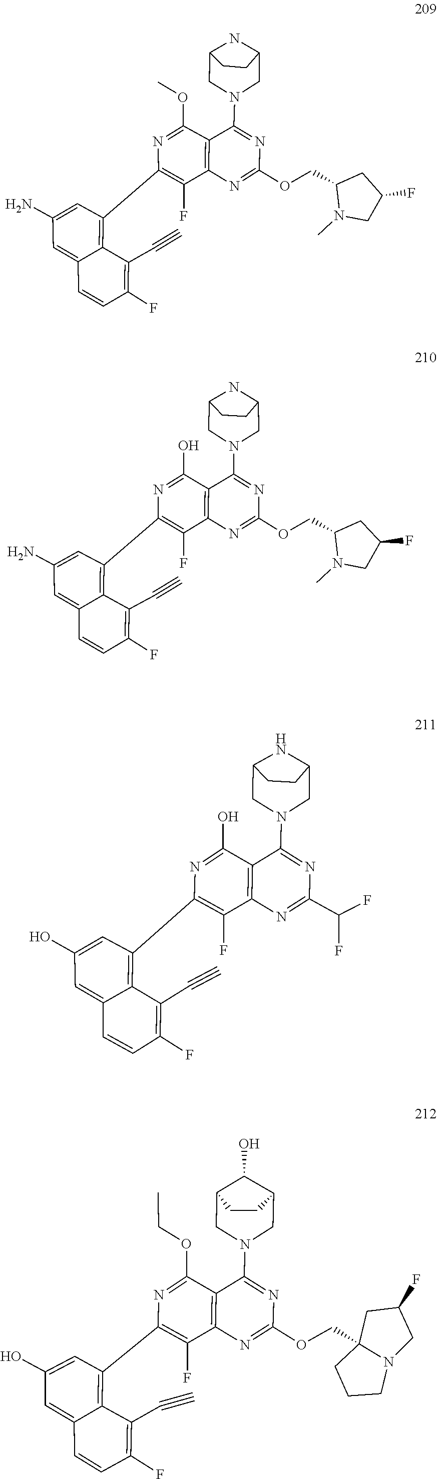

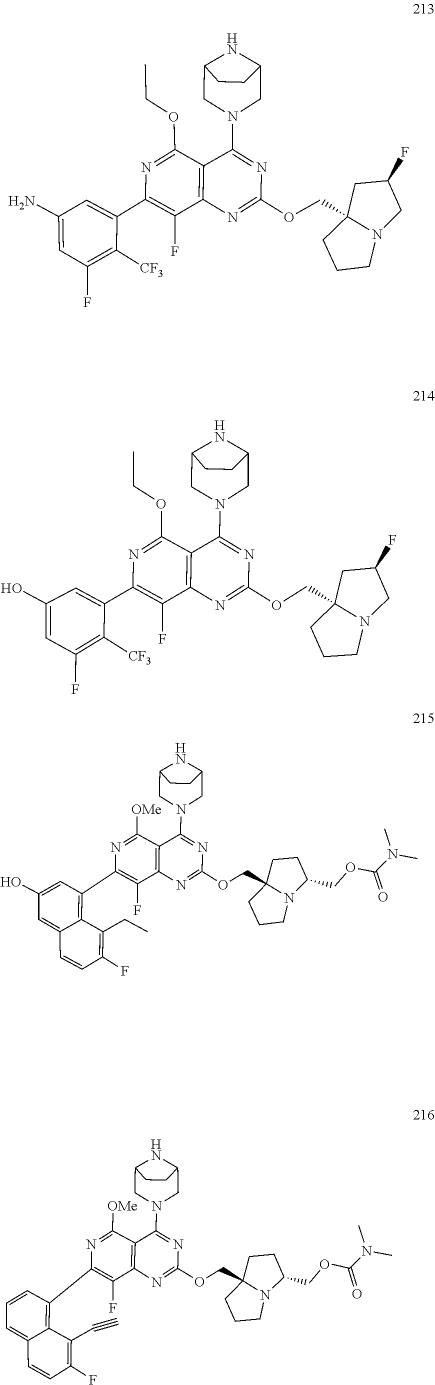 [SMILES: CCOc1nc(-c2cc(N)cc(F)c2C(F)(F)F)c(F)c2nc(OC[C@@]34CCCN3C[C@H](F)C4)nc(N3CC4CCC(C3)N4)c12 ; CCOc1nc(-c2cc(O)cc(F)c2C(F)(F)F)c(F)c2nc(OC[C@@]34CCCN3C[C@H](F)C4)nc(N3CC4CCC(C3)N4)c12 ; CCc1c(F)ccc2cc(O)cc(-c3nc(OC)c4c(N5CC6CCC(C5)N6)nc(OC[C@]56CCCN5[C@@H](COC(=O)N(C)C)CC6)nc4c3F)c12 ; C#Cc1c(F)ccc2cccc(-c3nc(OC)c4c(N5CC6CCC(C5)N6)nc(OC[C@]56CCCN5[C@@H](COC(=O)N(C)C)CC6)nc4c3F)c12]

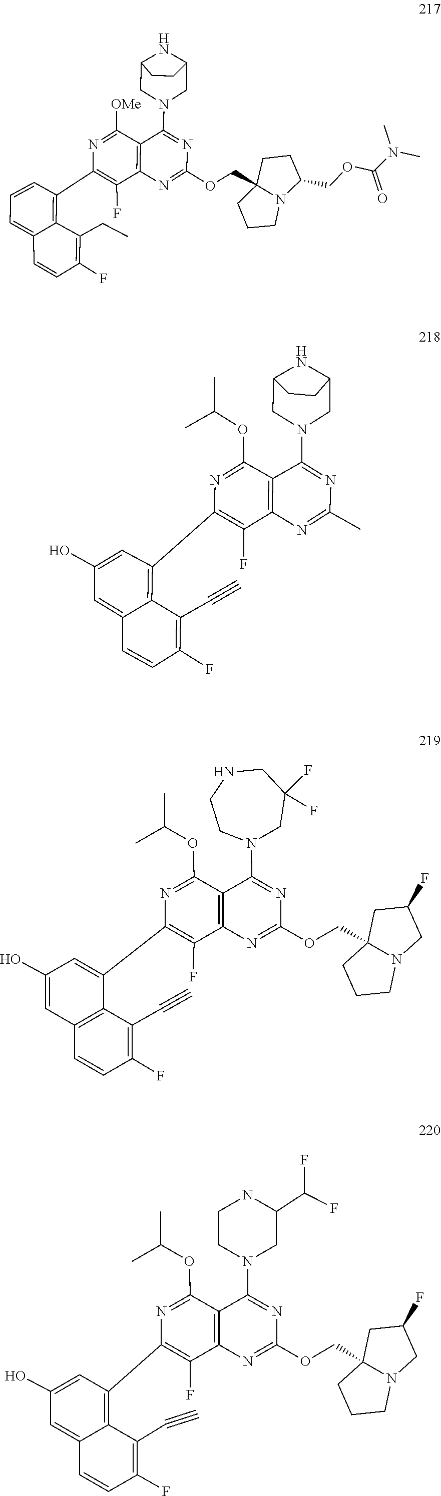

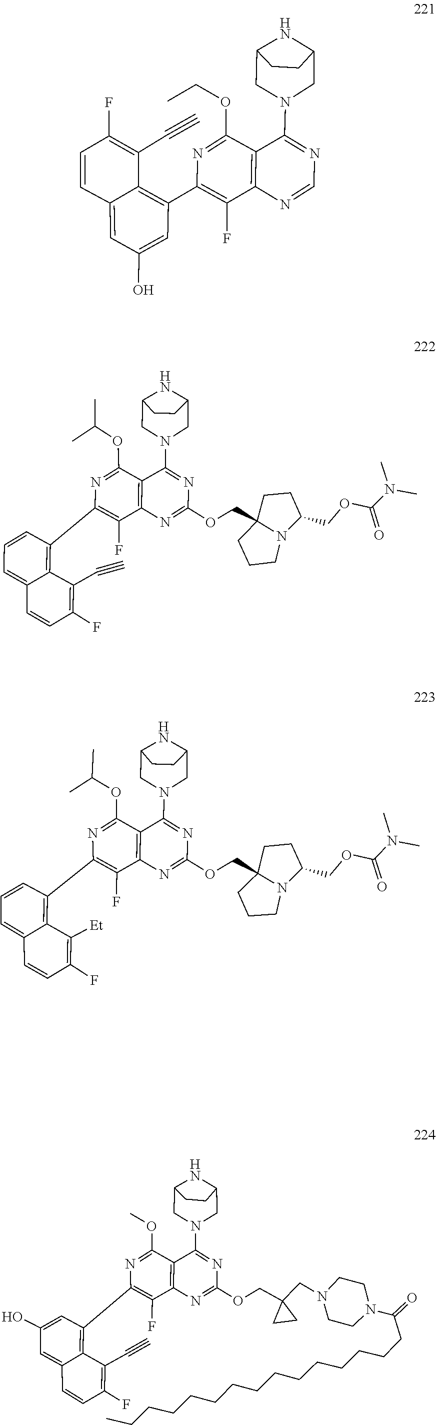 [SMILES: C#Cc1c(F)ccc2cc(O)cc(-c3nc(OCC)c4c(N5CC6CCC(C5)N6)ncnc4c3F)c12 ; C#Cc1c(F)ccc2cccc(-c3nc(OC(C)C)c4c(N5CC6CCC(C5)N6)nc(OC[C@]56CCCN5[C@@H](COC(=O)N(C)C)CC6)nc4c3F)c12 ; CCc1c(F)ccc2cccc(-c3nc(OC(C)C)c4c(N5CC6CCC(C5)N6)nc(OC[C@]56CCCN5[C@@H](COC(=O)N(C)C)CC6)nc4c3F)c12 ; C#Cc1c(F)ccc2cc(O)cc(-c3nc(OC)c4c(N5CC6CCC(C5)N6)nc(OCC5(CN6CCN(C(=O)CCCCCCCCCCCCCCC)CC6)CC5)nc4c3F)c12]

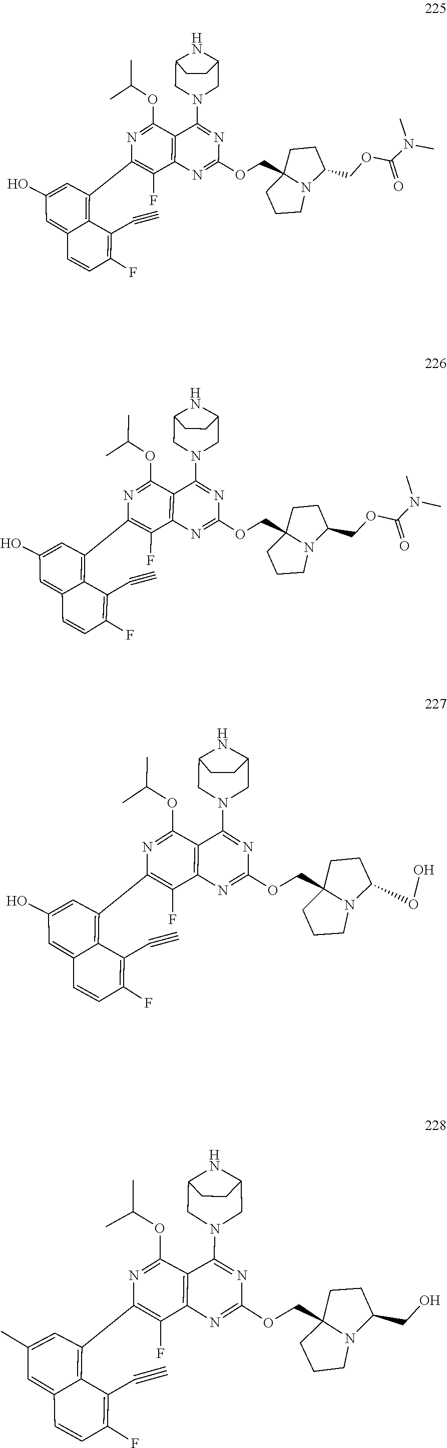

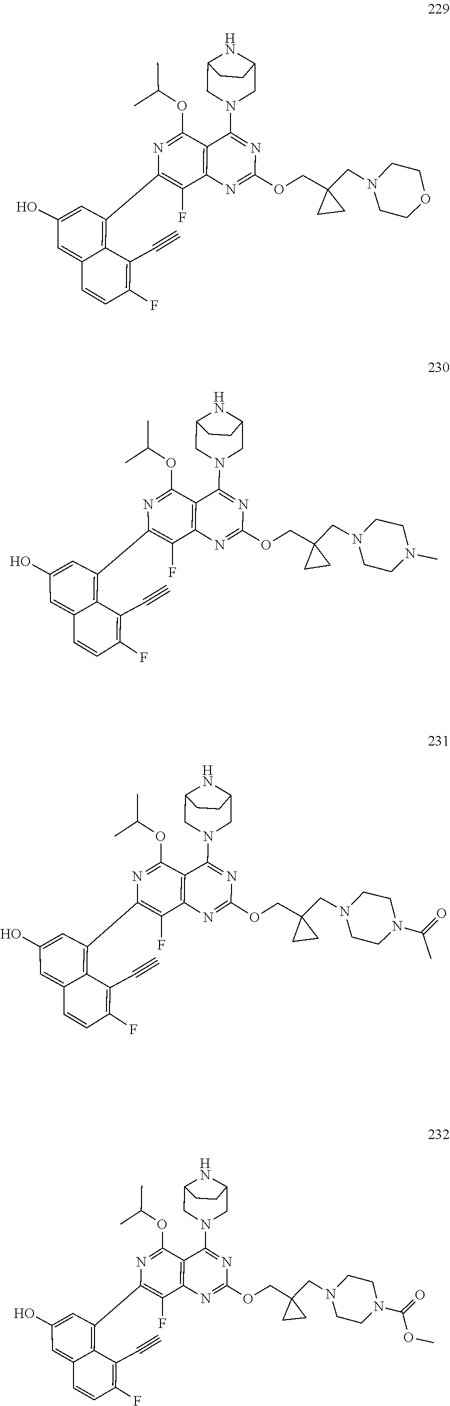 [SMILES: C#Cc1c(F)ccc2cc(O)cc(-c3nc(OC(C)C)c4c(N5CC6CCC(C5)N6)nc(OCC5(CN6CCOCC6)CC5)nc4c3F)c12 ; C#Cc1c(F)ccc2cc(O)cc(-c3nc(OC(C)C)c4c(N5CC6CCC(C5)N6)nc(OCC5(CN6CCN(C)CC6)CC5)nc4c3F)c12 ; C#Cc1c(F)ccc2cc(O)cc(-c3nc(OC(C)C)c4c(N5CC6CCC(C5)N6)nc(OCC5(CN6CCN(C(C)=O)CC6)CC5)nc4c3F)c12 ; C#Cc1c(F)ccc2cc(O)cc(-c3nc(OC(C)C)c4c(N5CC6CCC(C5)N6)nc(OCC5(CN6CCN(C(=O)OC)CC6)CC5)nc4c3F)c12]

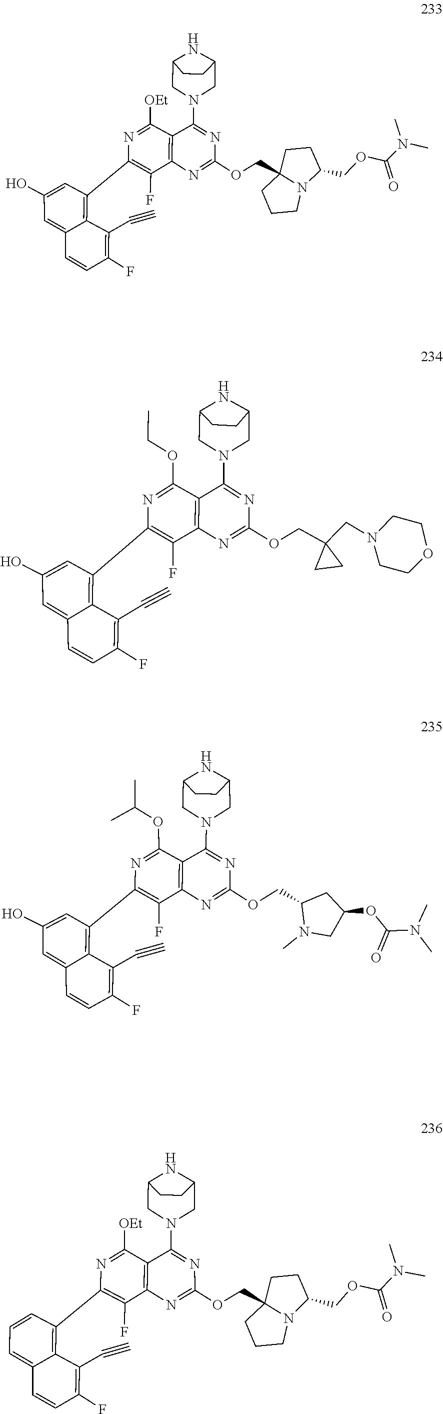 [SMILES: C#Cc1c(F)ccc2cc(O)cc(-c3nc(OCC)c4c(N5CC6CCC(C5)N6)nc(OC[C@]56CCCN5[C@@H](COC(=O)N(C)C)CC6)nc4c3F)c12 ; C#Cc1c(F)ccc2cc(O)cc(-c3nc(OCC)c4c(N5CC6CCC(C5)N6)nc(OCC5(CN6CCOCC6)CC5)nc4c3F)c12 ; C#Cc1c(F)ccc2cc(O)cc(-c3nc(OC(C)C)c4c(N5CC6CCC(C5)N6)nc(OC[C@@H]5C[C@@H](OC(=O)N(C)C)CN5C)nc4c3F)c12 ; C#Cc1c(F)ccc2cccc(-c3nc(OCC)c4c(N5CC6CCC(C5)N6)nc(OC[C@]56CCCN5[C@@H](COC(=O)N(C)C)CC6)nc4c3F)c12]

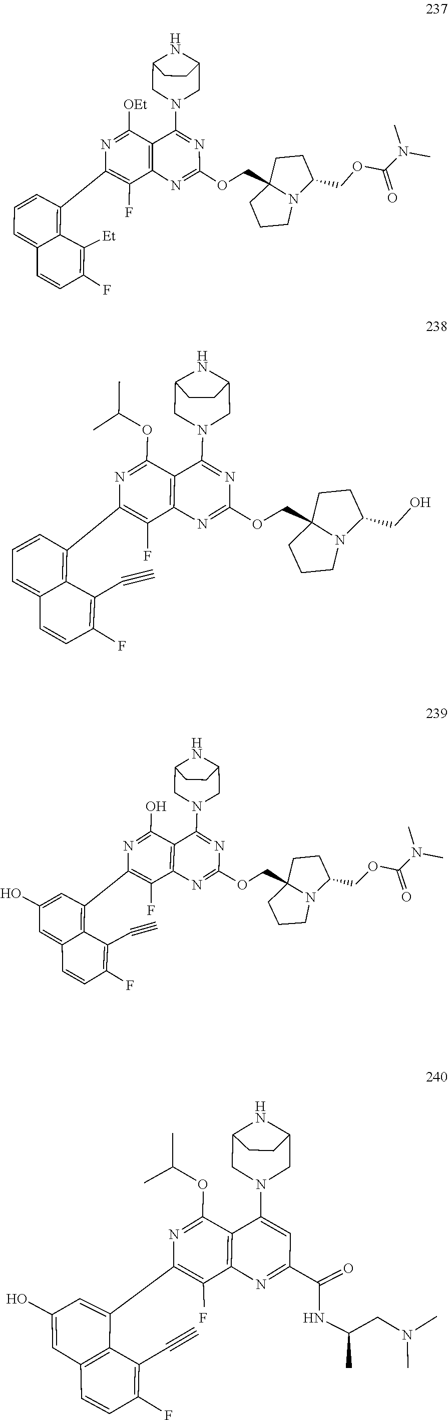

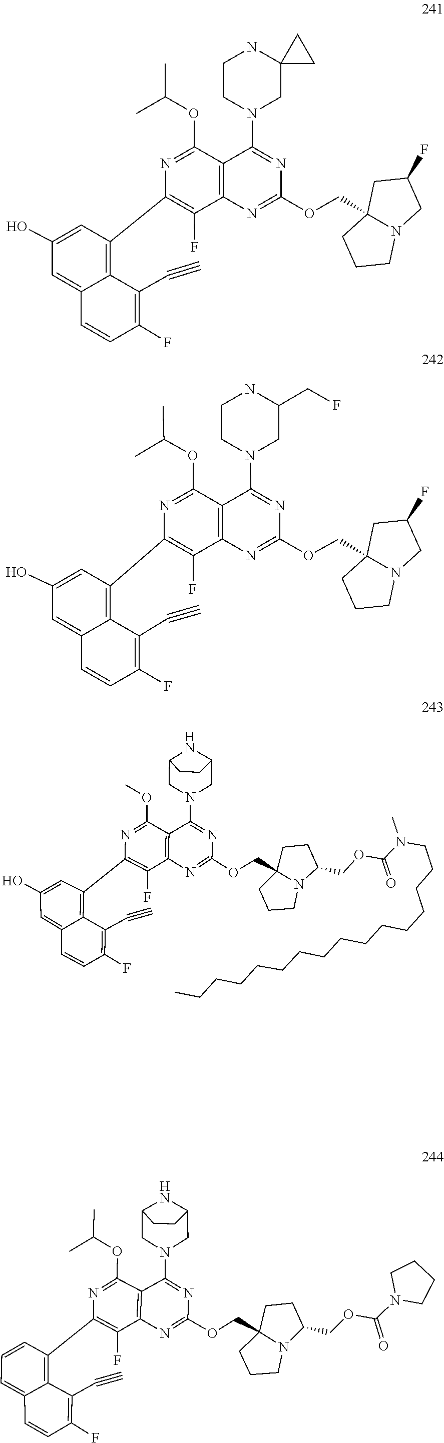

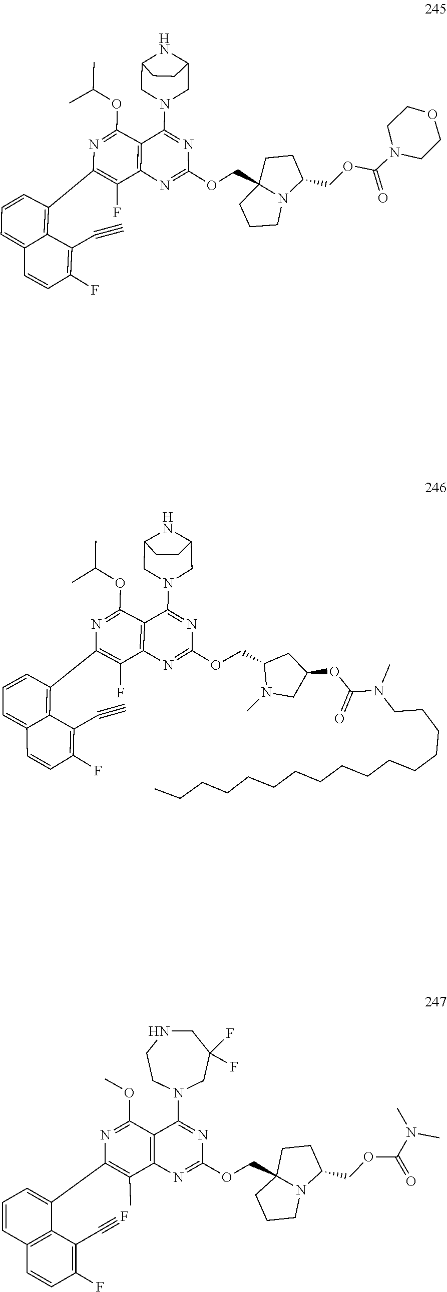

Exemplary synthesis and characterization of the above Compound Nos. I-247 are shown in the Examples section. For the specific compounds Nos. I-247, when it is labeled as “trans”, in the exemplary synthesis of such compound as shown in the Examples section, the compound may be prepared in a racemic form, which can be separated into two enantiomers, including the as-drawn enantiomer, or either or both of the two enantiomers can be prepared through chiral synthesis, in view of the present disclosure.

In some embodiments, to the extent applicable, the genus of compounds in the present disclosure also excludes any of the compounds specifically prepared and disclosed prior to this disclosure.

### Method of Synthesis

The compounds of the present disclosure can be readily synthesized by those skilled in the art in view of the present disclosure. Exemplified syntheses are also shown in the Examples section.

The following synthetic process of Formula I is illustrative, which can be applied similarly by those skilled in the art for the synthesis of compounds of Formula A, II, III, IV, or V, by using a proper synthetic starting material or intermediate. In some embodiments, the present disclosure also provides synthetic methods and synthetic intermediates for preparing the compounds of Formula I, A, II, III, IV, or V, as represented by the schemes herein, including those shown in the Examples section.

As shown in Scheme 1, compounds of Formula I can typically be synthesized through a series of coupling reactions. In some embodiments, a compound S-1 can be coupled with a R² donor S-2. Depending on the nature of R², this coupling can be carried out with or without a transition metal catalyst. In some embodiments, R²-M² can replace Lg¹ to form an O—C or N—C bond, wherein Lg¹ can be a leaving group described herein such as halogen (e.g., Cl), to produce compound S-3, typically, under basic conditions in an aprotic polar solvent. In some embodiments, M² is hydrogen. Compound S-3 can then be converted into Formula I by reacting with S-4. R¹-M¹ in S-4 typically includes a —OH, or —NH functional group, for example, M¹ can be hydrogen, such that it can react with S-3 to replace the leaving group Lg² to form an O—C or N—C bond. The leaving group Lg² can be a halogen (e.g., Cl) or another leaving group described herein such as methylsulfoxide, methylsulfone, etc. Other coupling sequences are also suitable. For example, in some embodiments according to Scheme 1, R¹ can be introduced first prior to introducing R² group. Exemplary reaction conditions for converting a compound of S-1 into a compound of Formula I are shown in the Examples section. The variables R¹, R², R³, J¹, J², J³, J⁴, and J⁵ in the formulae S-1, S-2, S-3, and S-4 of Scheme 1 include any of those defined hereinabove in connection with Formula I (e.g., any of the sub-formulae of Formula I) and protected derivatives thereof, when applicable. When a protecting group is used in the synthesis, for example, when a protected R² group is used in S-3, those skilled in the art would understand that the synthetic sequence also includes a deprotection step, e.g., after the coupling with S-4, to synthesize the compound of Formula I.

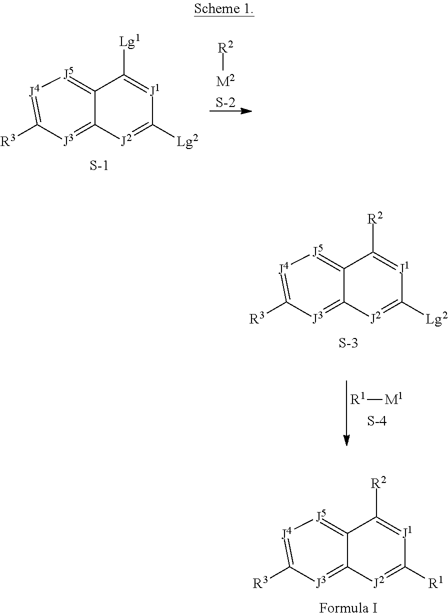

Compounds of Formula I can also be prepared through a slightly different coupling sequence. For example, as shown in Scheme 2, the synthesis can include coupling of a compound of S-5 with S-4 to form a compound of S-6. R¹-M¹ in S-4 typically includes a —OH, or —NH functional group, for example, M¹ can be hydrogen, such that it can react with S-5 to replace the leaving group Lg² to form an O—C or N—C bond. The leaving group Lg² can be a halogen or another leaving group described herein such as methylsulfoxide, methylsulfone, etc. The compound of S-6 can then react with a compound of S-7, R³-M³ to provide the compound of Formula I. Typically, the Lg³ in the compound of S-6 can be activated into a leaving group first before reacting with R³-M³ to yield the compound of Formula I. For example, in some embodiments, Lg³ in S-6 is hydroxyl or a protected hydroxyl group, which can be first converted into a leaving group such as a halide or a sulfonate, such as trifluoromethanesulfonate, which can then undergo a cross coupling reaction with the compound of S-7, R³-M³. Typically, M³ can be hydrogen, a metal (such as Zn²⁺), boronic acid or ester, tributyltin, etc., and the cross coupling is typically a transition metal catalyzed coupling reaction, such as a palladium catalyzed coupling reaction as exemplified herein. Other coupling sequences are also suitable. For example, in some embodiments according to Scheme 2, R³ can be introduced first prior to introducing R¹ group. In some embodiments, Lg² can also be a precursor to a suitable leaving group for coupling with a R¹ donor S-4. For example, in some embodiments, Lg² can be —S-Me, which can be oxidized first into —S(O)-Me or —S(O)₂Me before reacting with S-4 to introduce the R¹ group. Exemplary reaction conditions for converting a compound of S-5 into a compound of Formula I are shown in the Examples section, see e.g., Example 2. The variables R¹, R², R³, J¹, J², J³, J⁴, and J⁵ in the formulae S-4, S-5, S-6, and S-7 of Scheme 2 include any of those defined hereinabove in connection with Formula I (e.g., any of the sub-formulae of Formula I) and protected derivatives thereof, when applicable. When a protecting group is used in the synthesis, for example, when a protected R² group is used in S-6, those skilled in the art would understand that the synthetic sequence also includes a deprotection step, e.g., after the coupling with S-7, to synthesize the compound of Formula I.

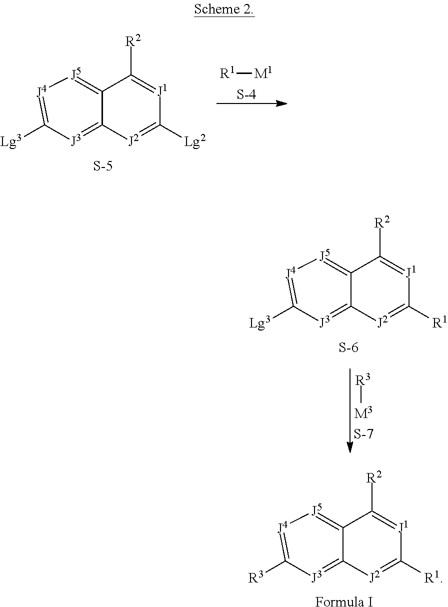

Suitable coup ing partners such as S-1, S-2, S-4, S-5, or S-7 can be prepare by methods known in the art or methods in view of the present disclosure, see e.g., the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more of the compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof, e.g., in a therapeutically effective amount. In any of the embodiments described herein, the pharmaceutical composition can comprise a therapeutically effective amount of a compound selected from compound Nos. I-247, or a pharmaceutically acceptable salt thereof.

The pharmaceutical composition can also be formulated for delivery via any of the known routes of delivery, which include but are not limited to oral, parenteral, inhalation, etc.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof). In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, which can depend on the recipient of the treatment, the disease or disorder being treated and the severity thereof, the composition containing the compound, the time of administration, the route of administration, the duration of treatment, the compound potency (e.g., for inhibiting KRAS G12D), its rate of clearance and whether or not another drug is co-administered.

For veterinary use, a compound of the present disclosure can be administered as a suitably acceptable formulation in accordance with normal veterinary practice. The veterinarian can readily determine the dosing regimen and route of administration that is most appropriate for a particular animal.

In some embodiments, all the necessary components for the treatment of KRAS-related disorder using a compound of the present disclosure either alone or in combination with another agent or intervention traditionally used for the treatment of such disease can be packaged into a kit. Specifically, in some embodiments, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound of the present disclosure, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

### Method of Treatment

Compounds of the present disclosure are useful as therapeutic active substances for the treatment and/or prophylaxis of diseases or disorders that are associated with RAS, e.g., KRAS^{G12D}.

In some embodiments, the present disclosure provides a method of inhibiting RAS-mediated cell signaling comprising contacting a cell (e.g., a cancer cell) with an effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof). Inhibition of RAS-mediated signal transduction can be assessed and demonstrated by a wide variety of ways known in the art. Non-limiting examples include a showing of (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

In some embodiments, the present disclosure provides a method of inhibiting KRAS^{G12D}, HRAS^{G12D}, and/or NRAS^{G12D} in a cell, e.g., a cancer cell, the method comprising contacting the cell with an effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof).

In some embodiments, the present disclosure provides a method of inhibiting KRAS mutant protein in a cell, e.g., a cancer cell, such as inhibiting KRAS^{G12D} in a cell, the method comprising contacting the cell with an effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, 1-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof).

In some embodiments, the present disclosure provides a method of inhibiting proliferation of a cell population (e.g., a cancer cell population), the method comprising contacting the cell population with an effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, T-16-E2, I-17, I-18, I-19, I-20, I-21, I-22, I-1-A, T-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof). In some embodiments, the inhibition of proliferation is measured as a decrease in cell viability of the cell population.

In some embodiments, the present disclosure provides a method of treating cancer in a subject, the method comprising administering to the subject a therapeutically effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein. In some embodiments, the cancer is a pancreatic cancer, lung cancer, colorectal cancer, endometrial cancer, appendix cancer, cholangiocarcinoma, bladder urothelial cancer, ovarian cancer, gastric cancer, breast cancer, bile duct cancer, and/or a hematologic malignancy. In some embodiments, the subject has a mutation of KRAS^{G12D}, HRAS^{G12D} and/or NRAS^{G12D}.

In some embodiments, the present disclosure provides a method of treating cancer metastasis or tumor metastasis in a subject, the method comprising administering to the subject a therapeutically effective amount of one or more compounds of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein.

In some embodiments, the present disclosure provides a method of treating a disease or disorder, e.g., a cancer associated with G12D mutation of KRAS, HRAS and/or NRAS, such as a cancer associated with KRAS^{G12D}, in a subject in need thereof. In some embodiments, the method comprises administering to the subject a therapeutically effective amount of a compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a therapeutically effective amount of a pharmaceutical composition described herein.

In some embodiments, a method treating cancer is provided, the method comprising administering to a subject in need thereof an effective amount of any of the compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, 1-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a pharmaceutical composition comprising the compound of the present disclosure. In some embodiments, the cancer comprises a G12D mutation of KRAS, HRAS and/or NRAS, e.g., a KRAS-G12D mutation. Determining whether a tumor or cancer comprises a G12D mutation of KRAS, HRAS and/or NRAS is known in the art, either by a PCR kit or using DNA sequencing. In various embodiments, the cancer can be pancreatic, colorectal, lung, and/or endometrial cancer. In some embodiments, the cancer is appendix cancer, cholangiocarcinoma, bladder urothelial cancer, ovarian cancer, gastric cancer, breast cancer, and/or bile duct cancer. In some embodiments, the cancer is a hematological malignancy (e.g., acute myeloid leukemia).

In some embodiments the present disclosure provides a method of treating a disease or disorder mediated by a Ras mutant protein (such as K-Ras, H-Ras, and/or N-Ras) in a subject in need thereof, the method comprising: a) determining if the subject has a Ras mutation; and b) if the subject is determined to have the Ras mutation, then administering to the subject a therapeutically effective amount of at least one compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a pharmaceutical composition described herein. In some embodiments, the disease or disorder is cancer, for example, lung cancer (e.g., non-small cell lung cancer), pancreatic cancer, colorectal cancer, endometrial cancer, appendix cancer, cholangiocarcinoma, bladder urothelial cancer, ovarian cancer, gastric cancer, breast cancer, bile duct cancer and/or hematological malignancy such as acute myeloid leukemia. In some embodiments, the disease or disorder is MYH associated polyposis.

In some embodiments the present disclosure provides a method of treating a disease or disorder (e.g., a cancer described herein) in a subject in need thereof, wherein the method comprises determining if the subject has a G12D mutation of KRAS, HRAS and/or NRAS, e.g., KRAS^{G12D} mutation, and if the subject is determined to have the KRAS, HRAS and/or NRAS^{G12D} mutation, e.g., KRAS G12D mutation, then administering to the subject a therapeutically effective dose of at least one compound of the present disclosure (e.g., a compound of Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, or a pharmaceutically acceptable salt thereof) or a pharmaceutical composition comprising the at least one compound of the present disclosure.

G12D mutation of KRAS, HRAS and/or NRAS has also been identified in hematological malignancies (e.g., cancers that affect blood, bone marrow and/or lymph nodes). Accordingly, certain embodiments are directed to a method of treating hematological malignancy in a subject in need thereof, the method typically comprises administration of a compound of the present disclosure (e.g., in the form of a pharmaceutical composition) to the subject. Such malignancies include, but are not limited to leukemias and lymphomas, such as Acute lymphoblastic leukemia (ALL), Acute myeloid leukemia (AML), Chronic lymphocytic leukemia (CLL), small lymphocytic lymphoma (SLL), Chronic myelogenous leukemia (CML), Acute monocytic leukemia (AMoL) and/or other leukemias. In some embodiments, the hematological malignancy can also include lymphomas such as Hodgkins lymphoma or non-Hodgkins lymphoma, plasma cell malignancies such as multiple myeloma, mantle cell lymphoma, and Waldenstrom's macroglubunemia.

Compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, and/or immunotherapy. In some embodiments, compounds of the present disclosure can also be co-administered with an additional pharmaceutically active compound, either concurrently or sequentially in any order, to a subject in need thereof (e.g., a subject having a cancer associated with KRAS^{G12D} mutation as described herein). In some embodiments, the additional pharmaceutically active compound can be a targeted agent (e.g. MEK inhibitor), a a chemotherapeutic agent (e.g. cisplatin or docetaxel), a therapeutic antibody (e.g. anti-PD-1 antibody), etc. Any of the known therapeutic agents can be used in combination with the compounds of the present disclosure. In some embodiments, compounds of the present disclosure can also be used in combination with a radiation therapy, hormone therapy, cell therapy, surgery and/or immunotherapy, which therapies are well known to those skilled in the art.

Many chemotherapeutics are presently known in the art and can be used in combination with the compounds of the present disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), venetoclax, and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; gemcitabine; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4 (5)-imidazoles, 4-hydroxytamoxifen, trioxifene, keoxifene, onapristone, and toremifene (Fareston); and anti-androgens such as flutamide, nilutamide, bicalutamide, leuprolide, and goserelin; chlorambucil; 6-thioguanine; mercaptopurine; methotrexate; pemetrexed; platinum analogs such as cisplatin, carboplatin and oxaliplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

Where desired, the compounds or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, Afatinib, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, Zosuquidar.

The compounds of the present disclosure may also be used in combination with an additional pharmaceutically active compound that disrupts or inhibits RAS-RAF-ERK or PI3K-AKT-TOR signaling pathways. In other such combinations, the additional pharmaceutically active compound is a PD-1 and PD-L1 antagonist. The compounds or pharmaceutical compositions of the disclosure can also be used in combination with an amount of one or more substances selected from EGFR inhibitors, CDK inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, anti-4-1BB (CD137) agonists, anti-GITR agonists, CAR-T cells, and BiTEs.

Exemplary anti-PD-1 or anti-PDL-1 antibodies and methods for their use are described by Goldberg et al., Blood 110(1):186-192 (2007), Thompson et al., Clin. Cancer Res. 13(6):1757-1761 (2007), and Korman et al., International Application No. PCT/JP2006/309606 (publication no. WO 2006/121168 A1), each of which are expressly incorporated by reference herein, include: pembrolizumab (Keytruda®), nivolumab (Opdivo®), Yervoy™ (ipilimumab) or Tremelimumab (to CTLA-4), galiximab (to B7.1), M7824 (a bifunctional anti-PD-L1/TGF-β Trap fusion protein), AMP224 (to B7DC), BMS-936559 (to B7-H1), MPDL3280A (to B7-H1), MEDI-570 (to ICOS), AMG 404, AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4). Immune therapies also include genetically engineered T-cells (e.g., CAR-T cells) and bispecific antibodies (e.g., BiTEs). Non-limiting useful additional agents also include anti-EGFR antibody and small molecule EGFR inhibitors such as cetuximab (Erbitux), panitumumab (Vectibix), zalutumumab, nimotuzumab, matuzumab, gefitinib, erlotinib, lapatinib, osimertinib, etc. Non-limiting useful additional agents also include CDK inhibitors such as CDK4/6 inhibitors, such as palbociclib, abemaciclib, ribociclib, dinaciclib, etc. Non-limiting useful additional agents also include MEK inhibitors such as trametinib and binimetinib. Non-limiting useful additional agents also include SHP2 inhibitors such as TNO155. RMC-4630 and RLY-1971.

The administering herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Dosing regimen including doses can vary and can be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable atoms or groups for the variables herein are independently selected. The definitions of the variables can be combined. Using Formula I as an example, any of the definitions of one of R¹, R², R³, J¹, J², J³, J⁴, and J⁵ in Formula I can be combined with any of the definitions of the others of R¹, R², R³, J¹, J², J³, J⁴, and J⁵ in Formula I. Such combination is contemplated and within the scope of the present disclosure.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds of the present disclosure can comprise one or more asymmetric centers and/or axial chirality, and thus can exist in various isomeric forms, e.g., enantiomers and/or diastereomers. For example, the compounds described herein can be in the form of an individual enantiomer, diastereomer, atropisomer, or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. In embodiments herein, unless otherwise obviously contrary from context, when a stereochemistry is specifically drawn, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s). The presence and/or amounts of stereoisomers can be determined by those skilled in the art in view of the present disclosure, including through the use of chiral HPLC.

Compounds of the present disclosure can have atropisomers. In any of the embodiments described herein, when applicable, the compound of the present disclosure can exist as a mixture of atropisomers in any ratio. In some embodiments, when applicable, the compound can exist as an isolated individual atropisomer substantially free (e.g., with less than 20%, less than 10%, less than 5%, less than 1%, by weight, by HPLC area, or both, or with a non-detectable amount) of the other atropisomer(s). The Examples section shows some exemplary isolated atropisomers of compounds of the present disclosure. As understood by those skilled in the art, when the rotation is restricted around a single bond, e.g., a biaryl single bond, a compound may exist in a mixture of atropisomers with each individual atropisomer isolable.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” or “compound(s) of the present invention” refers to any of the compounds described herein according to Formula I (e.g., Formula I-1, I-2, I-3, I-4, I-5, I-6, I-7, I-8, I-9, I-10, I-11, I-12, I-13, I-14, I-23, I-24, I-15, I-16, I-16-E1, I-16-E2, I-17, I-18, I-19, I-20, I-21, I-22, I-1-A, I-2-A, I-3-A, I-4-A, I-5-A, I-6-A, I-9-A, I-9-B, I-9-C, I-9-D, I-9-E, I-9-F, I-9-G, I-10-A, I-2-B, I-2-C, I-4-B, or I-6-B), Formula A (e.g., Formula A-1), Formula II (e.g., Formula II-1, II-2, II-3, II-4, II-5, II-6, II-7, or II-8), Formula III (e.g., Formula III-1, III-2, III-1-A, or III-2-A), Formula IV (e.g., Formula IV-1), Formula V (e.g., Formula V-1), any of Compound Nos. I-247, isotopically labeled compound(s) thereof (such as a deuterated analog wherein one or more of the hydrogen atoms is substituted with a deuterium atom with an abundance above its natural abundance), possible stereoisomers thereof (including diastereoisomers, enantiomers, and racemic mixtures), geometric isomers thereof, atropisomers thereof, tautomers thereof, conformational isomers thereof, and/or pharmaceutically acceptable salts thereof (e.g., acid addition salt such as HCl salt or base addition salt such as Na salt). For the avoidance of doubt, Compound Nos. I-247 or Compounds 1-247 refers to the compounds described herein labeled as integers 1, 2, 3, . . . , 247, see for example the title compounds of Examples 1-82 and Table 1. For ease of description, synthetic starting materials or intermediates may be labeled with an integer (compound number) followed by a “-” and additional numeric values, such as 66-1, 66-2, etc., see examples for details. The labeling of such synthetic starting materials or intermediates should not be confused with the compounds labeled with an integer only without the “-” and additional numeric value. Hydrates and solvates of the compounds of the present disclosure are considered compositions of the present disclosure, wherein the compound(s) is in association with water or solvent, respectively. In some embodiments, the compound of the present disclosure can be any of those defined in claims 1-77 herein. In some embodiments, the compound of the present disclosure can be any of those defined in exemplary Embodiments 1-44 herein. In some embodiments, the compound of the present disclosure can be any of those defined in exemplary Embodiments 45-57 herein.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In one embodiment, the alkyl group is a C₁₋₄ alkyl group selected from methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, such as one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, such as one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein Rai is an alkyl. As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₄ haloalkyl group.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring.

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). Heterocyclyl or heterocyclic ring that has a ring size different from the 3-10 membered heterocyclyl is specified with a different ring size designation when applicable. Those skilled in the art would understand that such different ring-sized heterocyclyl is also a non-aromatic ring system having ring carbon atoms and 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). Heteroaryl that has a ring size different from the 5-10 membered heteroaryl is specified with a different ring size designation when applicable. Those skilled in the art would understand that such different ring-sized heteroaryl is also a 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur. In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzothiazolyl, benzisothiazolyl, benzothiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As commonly understood by those skilled in the art, alkylene, alkenylene, alkynylene, carbocyclylene, heterocyclylene, arylene, and heteroarylene refer to the corresponding divalent radicals of alkyl, alkenyl, alkynyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂—NR^{bb}P(═O)(OR^{cc})₂—NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 Rd groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rad groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rd groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₄ alkyl), —N(C₁₋₆ alkyl)C(═O)(C₁₋₄ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₄ alkyl, C₁₋₄ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, Salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{aa} groups, and wherein R^{aa}, R^{bb}, R^{cc}, and R^{dd} are as defined above.

In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂ wherein X⁻, R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, alkyl ethers or substituted alkyl ethers such as methyl, allyl, benzyl, substituted benzyls such as 4-methoxybenzyl, methoxymethyl (MOM), benzyloxymethyl (BOM), 2-methoxyethoxymethyl (MEM), etc., silyl ethers such as trymethylsilyl (TMS), triethylsilyl (TES), triisopropylsilyl (TIPS), t-butyldimethylsilyl (TBDMS), etc., acetals or ketals, such as tetrahydropyranyl (THP), esters such as formate, acetate, chloroacetate, dichloroacetate, trichloroacetate, trifluoroacetate, methoxyacetate, etc., carbonates, sulfonates such as methanesulfonate (mesylate), benzylsulfonate, and tosylate (Ts), etc.

The term “leaving group” is given its ordinary meaning in the art of synthetic organic chemistry, for example, it can refer to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

### Example 1 Synthesis of Compound 20

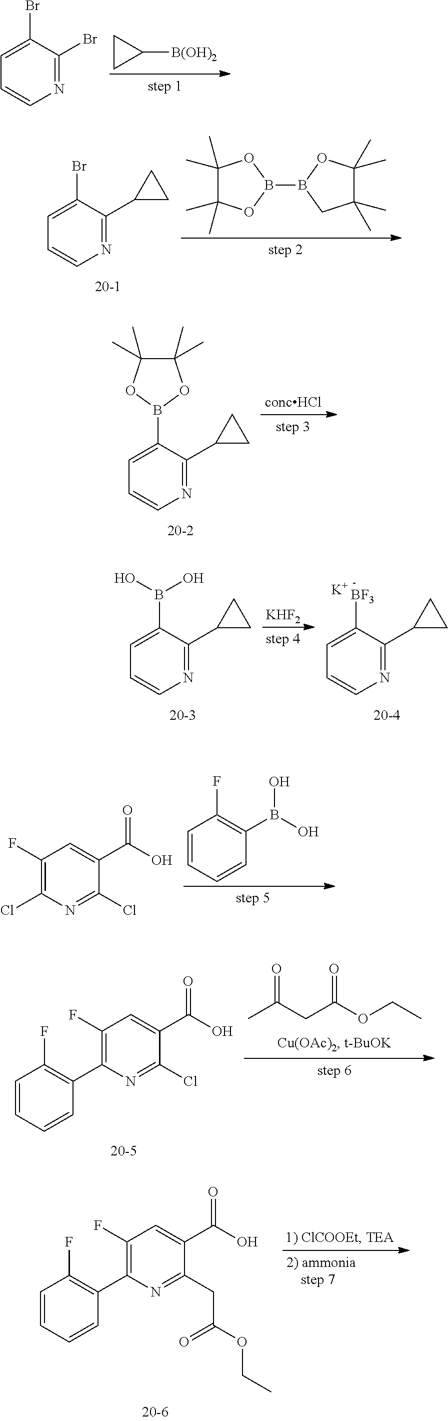

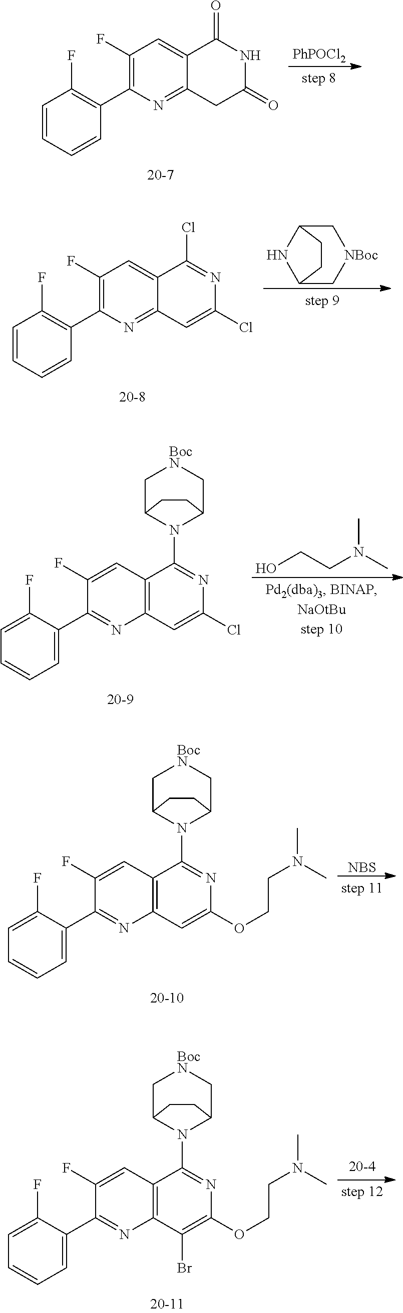

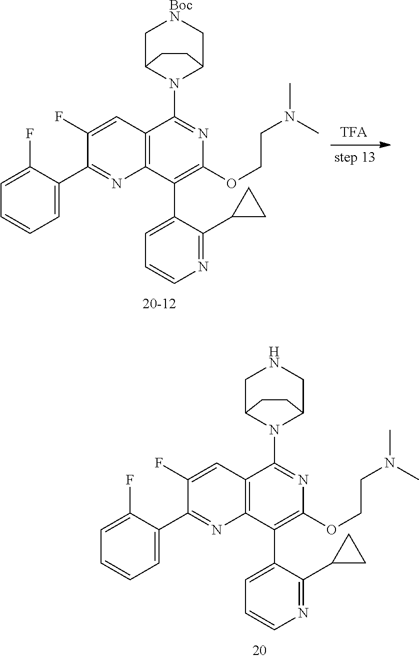 [SMILES: CN(C)CCOc1nc(N2C3CCC2CN(C(=O)OC(C)(C)C)C3)c2cc(F)c(-c3ccccc3F)nc2c1-c1cccnc1C1CC1 ; CN(C)CCOc1nc(N2C3CCC2CNC3)c2cc(F)c(-c3ccccc3F)nc2c1-c1cccnc1C1CC1]

Step 1: A mixture of 4-bromonaphthalen-2-ol (3.0 g, 13.4 mmol), bis(pinacolato)diboron (4.1 g, 16.1 mmol), Pd(dppf)Cl₂ (0.98 g, 1.35 mmol) and KOAc (3.9 g, 40.3 mmol) in 1,4-dioxane (30 mL) was stirred at 95° C. for 2 hours under nitrogen atmosphere. The mixture was cooled and diluted with water. The resulting mixture was extracted with ethyl acetate. The combined organic layers were washed with brine, dried over anhydrous Na₂SO₄ and filtered. The residue was purified by silica gel column chromatography (petroleum ether/ethyl acetate=10/1) to afford 20-1.

Step 2: A mixture of 20-1 (1.48 g, 7.5 mmol), 4,4,4′,4′,5,5,5′,5′-octamethyl-2,2′-bi(1,3,2-dioxaborolane) (2.86 g, 11.25 mmol), potassium acetate (1.47 g, 15 mmol) and [1,1′-bis(diphenylphosphino)ferrocene]dichloropalladium(II) (549 mg, 0.75 mmol) in 1,4-dioxane (50 mL) was stirred at 100° C. for 7 hours under nitrogen atmosphere. The mixture was cooled and diluted with water. The resulting mixture was extracted with ethyl acetate. The combined organic layers were washed with brine, dried over anhydrous Na₂SO₄ and filtered. The residue was purified by silica gel column chromatography (petroleum ether/ethyl acetate=5/1) to afford 20-2.

Step 3: A mixture of 20-2 (2 g, 8.16 mmol) and conc. HCl (12 mL) in methanol (4 mL) was stirred at 65° C. for 2 hours. The mixture was diluted with water and basified with NaOH solid to pH˜ 5 and extracted with ethyl acetate. The aqueous layer was concentrated and the residue was washed with 10% methanol in dichloromethane. The combined organic layers were concentrated and the residue was purified by reverse phase HPLC (acetonitrile with 0.05% of TFA in water: 0% to 5%) to afford 20-3.

Step 4: A mixture of 20-3 (980 mg, 3.54 mmol) and 4.5 M aq. KHF₂ (4.4 mL, 19.8 mmol) in methanol (10 mL) was stirred at room temperature for 0.5 hours. The mixture was concentrated and the residue was washed with hot acetone (80 mL) and filtered. The filtrate was concentrated and the residue was triturated with ethyl ether. Then the suspension was filtered and the filter cake was dried to afford 20-4.

Step 5: To a mixture of 2,6-dichloro-5-fluoronicotinic acid (10 g, 47.6 mmol), (2-fluorophenyl) boronic acid (12.3 g, 71.5 mmol) and sat. aq. NaHCO₃ solution (150 mL) in 300 mL of DME was added Pd(dppf)Cl₂ (2.7 g, 3.69 mmol). The mixture was stirred at 70° C. for 16 hours under nitrogen atmosphere. The mixture was cooled, diluted with water and extracted with ethyl acetate. The aqueous layer was acidified with conc. HCl to pH-1 and extracted with ethyl acetate. The combined organic layers were washed with brine, dried over anhydrous Na₂SO₄ and filtered. The residue was triturated with dichloromethane, filtered and the filter cake was dried to afford 20-5.

Step 6: To a solution of ethyl 3-oxobutanoate (4.97 g, 38.2 mmol) in DME (250 mL) was added t-BuOK (11.4 g, 102.0 mmol). The mixture was stirred at room temperature for 1 hour. Cu(OAc)₂ (1.86 g, 10.2 mmol) and 20-5 (7.7 g, 25.5 mmol) were added and the resulting mixture was stirred at 100° C. for 24 hours under nitrogen atmosphere. The mixture was diluted with water, acidified with 1 M HCl to pH-2 and extracted with ethyl acetate. The combined organic layers were dried over sodium sulfate, filtered and concentrated. The residue was purified by column chromatography on silica (dichloromethane/methanol=10/1) to afford 20-6.

Step 7: To a solution of 20-6 (4.45 g, 13.86 mmol) and triethylamine (7.7 mL, 55.35 mmol) in tetrahydrofuran (40 mL) was added ethyl carbonochloridate (2.63 mL, 27.74 mmol) dropwise at 0° C. under N₂ atmosphere. The mixture was stirred at this temperature for 1 hour before ammonia (28%, 12 mL) was added dropwise and the resulting mixture was stirred at room temperature for 1 hour. The mixture was diluted with water and extracted with ethyl acetate. The combined organic layers were washed with brine, dried over sodium sulfate, filtered and concentrated. The residue was purified by silica gel column chromatography (dichloromethane/methanol=10/1) to afford 20-7.

Step 8: A solution of 20-7 (466 mg, 1.70 mmol) in PhPOCl₂ (5 mL) was stirred at 110° C. for 3 hours under nitrogen atmosphere. The mixture was cooled and diluted with ethyl acetate. The mixture was washed with brine, dried over anhydrous sodium sulfate, filtered and concentrated. The residue was purified by silica gel column chromatography (petroleum ether/ethyl acetate=10/1) to afford 20-8.

Step 9: A mixture of 20-8 (100 mg, 0.32 mmol), tert-butyl (1R,5S)-3,8-diazabicyclo [3.2.1] octane-3-carboxylate (204 mg, 0.96 mmol) and DIPEA (0.33 mL, 1.90 mmol) in DMSO (3 mL) was stirred at 90° C. for 18 hours. The mixture was cooled and diluted with ethyl acetate. The mixture was washed with brine, dried over anhydrous sodium sulfate, filtered and concentrated. The residue was purified by silica gel column chromatography (petroleum ether/ethyl acetate=2/1) to afford 20-9.

Step 10: A mixture of 20-9 (134 mg, 0.27 mmol), 2-(dimethylamino)ethan-1-ol (75 mg, 0.85 mmol), NaOt-Bu (95 mg, 0.99 mmol), Pd₂(dba)₃ (13 mg, 0.014 mmol) and BINAP (17.6 mg, 0.028 mmol) in toluene (4 mL) was stirred at 110° C. for 6 hours under nitrogen atmosphere. The mixture was concentrated and the residue was purified by silica gel column chromatography (dichloromethane/methanol=20/1) to afford 20-10.

Step 11: To a suspension of 20-10 (63 mg, 0.12 mmol) in acetonitrile (3.5 mL) was added a solution of NBS (22 mg, 0.12 mmol) in acetonitrile (0.5 mL) dropwise at −35° C. After being stirred for 2 min, the mixture was quenched with water and extracted with ethyl acetate. The combined organic layers were washed with brine, dried over anhydrous sodium sulfate, filtered and concentrated. The residue was purified by silica gel column chromatography (dichloromethane/methanol=20/1) to afford 20-11.

Step 12: A mixture of 20-11 (52 mg, 0.084 mmol), 20-4 (90 mg, 0.27 mmol), Pd(dtbpf)Cl₂ (11 mg, 0.017 mmol) and K₃PO₄ (62 mg, 0.29 mmol) in DMF (0.5 mL) and one drop of water was stirred at 90° C. for 1 hour under nitrogen atmosphere. The mixture was concentrated and the residue was purified by reverse phase HPLC (acetonitrile with 0.05% of TFA in water: 5% to 70%) to afford 20-12.

Step 13: To a solution of 20-12 (5 mg, 0.0076 mmol) in dichloromethane (3 mL) was added TFA (0.5 mL). The resulting mixture was stirred at room temperature for 1 hour. The mixture was concentrated and the residue was purified by prep-HPLC (acetonitrile with 0.05% of TFA in water: 5% to 70%) to afford 20 as a 3 eq of TFA salt. LCMS (ESI, m/z): [M+H]⁺=557.4; ¹H NMR (400 MHz, methanol-d₄, ppm): δ 8.51 (d, J=4.4 Hz, 1H), 8.38 (d, J=10.0 Hz, 1H), 8.29 (d, J=6.8 Hz, 1H), 7.75-7.72 (m, 1H), 7.51-7.48 (m, 2H), 7.28-7.20 (m, 2H), 4.81-4.74 (m, 2H), 3.78-3.73 (mn, 2H), 3.60-3.50 (mn, 2H), 3.43 (d, J=12 Hz, 2H), 3.30-3.28 (mn, 2H), 2.83 (s, 6H), 2.38-2.29 (mn, 2H), 2.14-2.09 (m, 2H), 2.04-1.99 (m, 1H), 1.12-0.98 (in, 4H).

### Example 2 Synthesis of Compound 43

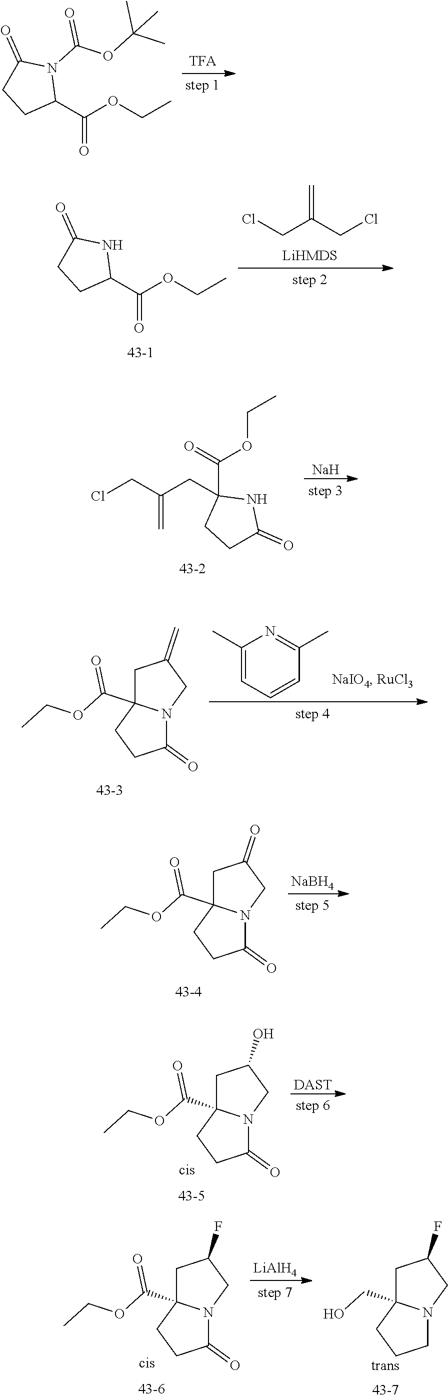

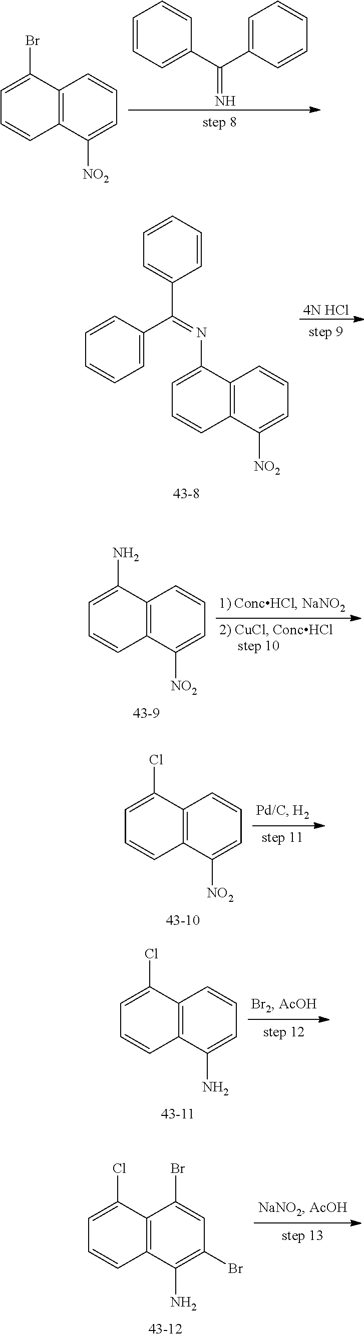

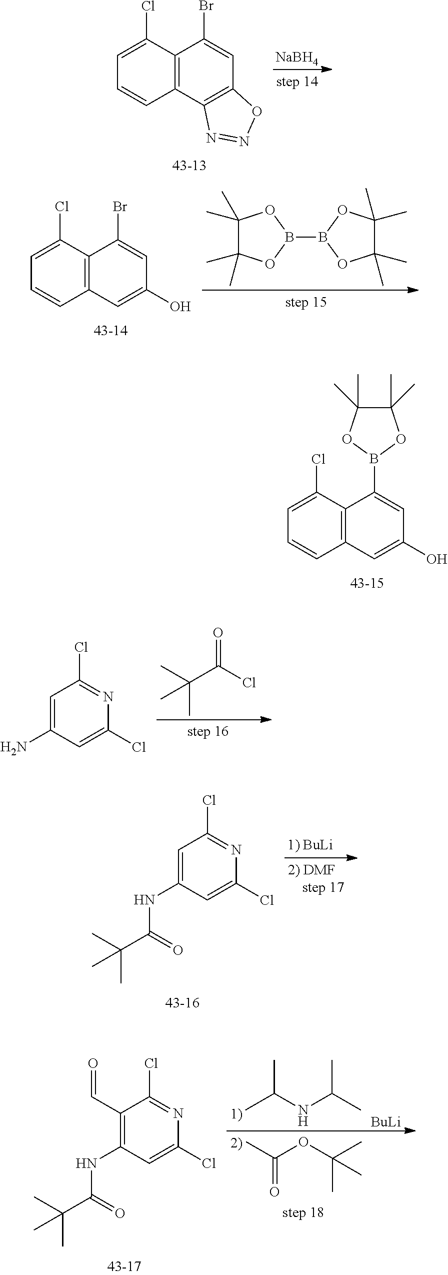

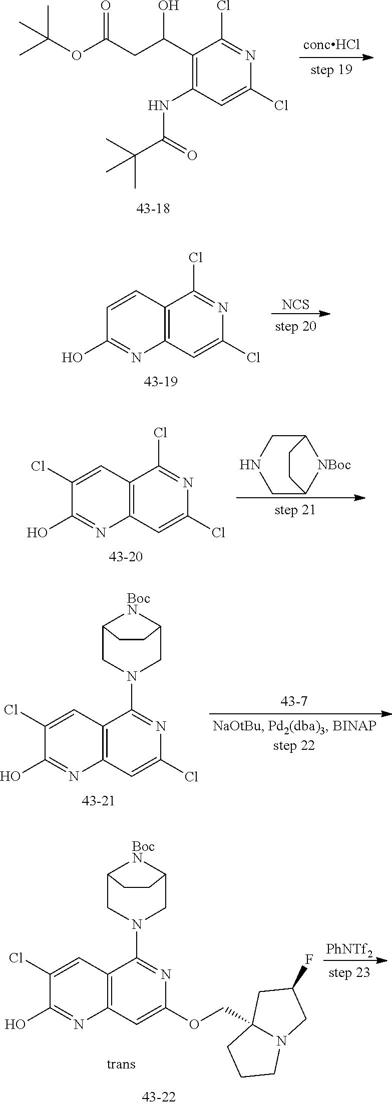

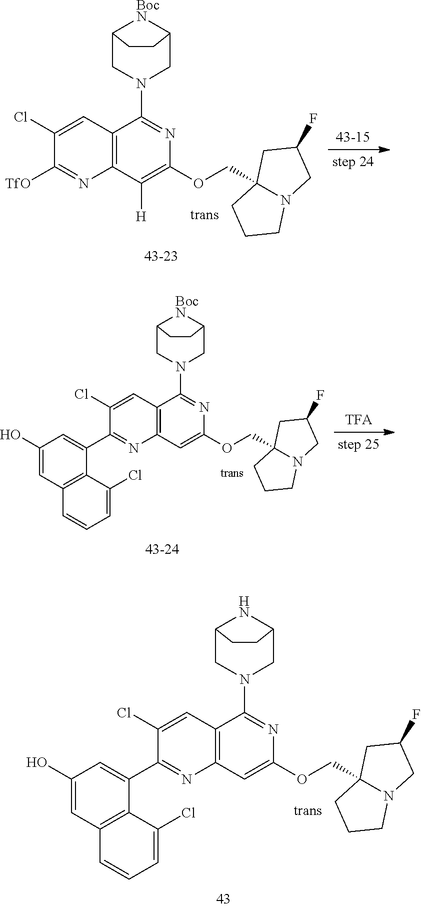 [SMILES: CC(C)(C)OC(=O)N1C2CCC1CN(c1nc(OC[C@@]34CCCN3C[C@H](F)C4)cc3nc(OS(=O)(=O)C(F)(F)F)c(Cl)cc13)C2 ; CC(C)(C)OC(=O)N1C2CCC1CN(c1nc(OC[C@@]34CCCN3C[C@H](F)C4)cc3nc(-c4cc(O)cc5cccc(Cl)c45)c(Cl)cc13)C2 ; Oc1cc(-c2nc3cc(OC[C@@]45CCCN4C[C@H](F)C5)nc(N4CC5CCC(C4)N5)c3cc2Cl)c2c(Cl)cccc2c1]

Step 1: To a solution of 1-(tert-butyl) 2-ethyl 5-oxopyrrolidine-1,2-dicarboxylate (100 g, 388.7 mmol) in dichloromethane (160 mL) was added trifluoroacetic acid (80 mL) slowly at room temperature. The mixture was stirred at room temperature for 16 hours, and then concentrated. The residue was diluted with sat. NaHCO₃ and extracted with ethyl acetate. The combined organic layers were washed with brine, dried over Na₂SO₄, filtered and concentrated to afford 43-1.

Step 2: To a solution of 43-1 (49 g, 311.8 mmol) and 3-chloro-2-(chloromethyl)prop-1-ene (100 g, 800 mmol) in tetrahydrofuran (200 mL) was added LiHMDS (655 mL, 1.0 M in tetrahydrofuran, 655 mmol) at −40° C. under nitrogen atmosphere. The mixture was stirred at room temperature for 2 hours. The reaction was quenched with sat. NH₄Cl. The mixture was extracted with ethyl acetate. The combined organic layers were washed with brine, dried over Na₂SO₄, filtered and concentrated. The residue was purified by column chromatography on silica gel (petroleum ether to petroleum ether/ethyl acetate=1/1) to afford 43-2.

Step 3: To a solution of sodium hydride (2.72 g, 68.1 mmol) in tetrahydrofuran (1 L) was added a solution of 43-2 (13.6 g, 55.35 mmol) in tetrahydrofuran (100 mL) dropwise at 0° C. under nitrogen atmosphere. Then the mixture was heated to reflux and stirred for 9 hours. The mixture was cooled to 0° C., quenched with water, and extracted with ethyl acetate. The combined organic layers were washed with brine, dried over Na₂SO₄, filtered and concentrated. The residue was purified by column chromatography on silica gel (petroleum ether to petroleum ether/ethyl acetate=1/1) to afford 43-3.

Step 4: To a solution of 43-3 (9.0 g, 43.15 mmol) in acetonitrile (245 mL) and dichloromethane (245 mL) was added 2,6-dimethylpyridine (9.25 g, 86.3 mmol), water (370 mL), and periodate sodium (36.9 g, 172.6 mmol) sequentially. A solution of Ruthenium (III) chloride (313 mg, 1.51 mmol) in water (40 mL) was added dropwise to the mixture. The mixture was stirred for 1 hour at room temperature before it was diluted with water and extracted with dichloromethane. The combined organic layers were washed with brine, dried over Na₂SO₄, filtered and concentrated. The residue was purified by column chromatography on silica gel (petroleum ether to petroleum ether/ethyl acetate=1/1) to afford 43-4.

Step 5: To a solution of 43-4 (10.6 g, 50.2 mmol) in methanol (100 mL) was added sodium borohydride (475 mg, 12.55 mmol) in portions at 0° C. under nitrogen atmosphere, and the mixture was stirred at 0° C. for 5 min. The mixture was concentrated and purified by column chromatography on silica gel (petroleum ether to ethyl acetate) to afford 43-5.

Step 6: To a solution of 43-5 (4.8 g, 22.6 mmol) in dichloromethane (50 mL) was added diethylaminosulfur trifluoride (4.1 g, 2.35 mmol) at −78° C. The mixture was stirred for at room temperature for 5 hours before it was quenched with methanol, diluted with water, and extracted with dichloromethane. The combined organic layers were washed with brine, dried over sodium sulfate, filtered and concentrated. The residue was purified by column chromatography on silica gel (petroleum ether to petroleum ether/ethyl acetate=1/1) to afford 43-6.

Step 7: To a solution of lithium aluminium hydride (1.25 g, 33 mmol) in tetrahydrofuran (33 mL) was added a solution of 43-6 (2.36 g, 11 mmol) in tetrahydrofuran (10 mL) at 0° C. under nitrogen atmosphere. The mixture was stirred at reflux for 2 hours, and then cooled to 0° C. Water (1.3 mL), 15% aqueous NaOH solution (1.3 mL) and water (3.9 mL) was added. The mixture was dried over sodium sulfate and filtered. The filtrate was concentrated to afford 43-7.

Step 8: A mixture of 5-bromo-1-nitro-naphthalene (25 g, 100 mmol), benzophenone imine (24 g, 130 mmol), Pd₂(dba)₃ (4.6 g, 5 mmol), XantPhos (2.9 g, 5 mmol) and Cs₂CO₃ (49 g, 150 mmol) in DMF (250 mL) was stirred at 100° C. for 5 hours under nitrogen atmosphere. Then, the mixture was filtered, and the filtrate was poured into water. The mixture was filtered and the filter cake was dried to afford 43-8.

Step 9: To a solution of 43-8 (31.3 g, 89 mmol) in dioxane (200 mL) was added 4N HCl (100 mL). The mixture was stirred at room temperature for 1 hour. Then the suspension was filtered and the filter cake was dried to afford 43-9.

Step 10: To a suspension of 43-9 (78.8 g, 350 mmol) in conc. HCl (350 mL) and water (175 mL) was added a solution of sodium nitrite (25.4 g, 367.5 mmol) in water (51 mL) at 0° C. over 30 min. The reaction mixture was added to a solution of CuCl (41.6 g, 420 mmol) in conc. HCl (131 mL) and water (175 mL) at room temperature over 1 hour. The mixture was diluted with water and filtered. The filter cake was dissolved in dichloromethane, and washed with water, sat. NaHCO₃ solution and brine. The organic layer was dried over anhydrous Na₂SO₄, filtered and concentrated to afford 43-10.

Step 11: A mixture of 43-10 (67.6 g, 327 mmol) and 5% Pd/C (13.5 g) in ethyl acetate (2.37 L) was stirred at room temperature overnight under H₂ atmosphere. The reaction mixture was filtered. The filtrate was concentrated and triturated with n-heptane to afford 43-11.

Step 12: To a solution of bromine (97.9 g, 613.1 mmol) in acetic acid (470 mL) was added a solution of 43-11 (49.5 g, 278.7 mmol) in acetic acid (200 mL) at room temperature. The mixture was stirred at 70° C. for 4 hours. The reaction mixture was cooled to room temperature and filtered. The filter cake was washed with acetic acid (120 mL) and then suspended in 20% NaOH (600 mL). The mixture was stirred at room temperature for 20 min and filtered. The solid was dissolved in dichloromethane, washed with brine, dried over anhydrous Na₂SO₄, filtered and concentrated to afford 43-12.

Step 13: To a solution of 43-12 (45.1 g, 134.3 mmol) in acetic acid (870 mL) and propionic acid (145 mL) was added sodium nitrite (13.0 g, 188.1 mmol) portion-wised at 5° C. The mixture was stirred at 5° C. for 1 hour before it was filtered and the filtrate was poured into water. The resulting mixture was filtered. The filter cake was dissolved in dichloromethane, washed with brine, dried over Na₂SO₄, filtered and concentrated to afford 43-13.

Step 14: To a suspension of 43-13 (30.6 g, 108.1 mmol) in ethanol (310 mL) was added sodium borohydride (8.17 g, 216.15 mmol) portion-wise at 5° C. The mixture was stirred at 5° C. for 1 hour, quenched with water (300 mL) and adjusted to around pH 5 with 1N HCl. The mixture was concentrated to remove the organic solvent. The resulting mixture was extracted with ethyl acetate. The combined organic layers were washed with brine, dried over anhydrous Na₂SO₄, filtered and concentrated. The residue was purified by column chromatography on silica gel (petroleum ether to petroleum ether/ethyl acetate=4/1) to afford 43-14.

Step 15: A mixture of 43-14 (6 g, 23.3 mmol), bis(pinacolato)diboron (11.84 g, 46.6 mmol), potassium acetate (6.85 g, 69.9 mmol), and Pd(dppf)Cl₂ (1.7 g, 2.33 mmol) in 1,4-dioxane (100 mL) was stirred at 95° C. for 7 hours under N₂ atmosphere. Then the mixture was diluted with water and extracted with ethyl acetate. The combined organic layers were washed with brine, dried over Na₂SO₄, filtered and concentrated. The residue was purified by column chromatography on silica gel (petroleum ether to petroleum ether/ethyl acetate=4/1) to afford 43-15.

Step 16: To a solution of 2,6-dichloropyridin-4-amine (27 g, 166 mmol) and triethylamine (50 g, 500 mmol) in dichloromethane (260 mL) was added pivaloyl chloride (24 g, 200 mmol) dropwise at 0° C. under N₂ atmosphere. After being stirred at room temperature for 5 hours, the mixture was washed with water, sat. aqueous sodium bicarbonate solution and brine. The organic layer was dried over sodium sulfate, filtered and concentrated. The residue was triturated with methyl tert-butyl ether to afford 43-16.

Step 17: To a solution of 43-16 (13 g, 53 mmol) in tetrahydrofuran (150 mL) was added n-butyllithium (2.5 M, 53 mL, 132.5 mmol) dropwise at −78° C. under N₂ atmosphere. The mixture was stirred at −78° C. for 3 hours. To above mixture was added N,N-dimethylformamide (11.6 g, 159 mmol) at −78° C. under N₂ atmosphere. The mixture was stirred at −78° C. for 0.5 hours. The reaction was quenched with sat. NH₄Cl solution and extracted with ethyl acetate. The combined organic layers were washed with water and brine, dried over sodium sulfate, filtered and concentrated. The residue was purified by column chromatography on silica gel (petroleum ether to petroleum ether/dichloromethane=3/1) to afford 43-17.

Step 18: To a solution of diisopropylamine (8.67 g, 85.8 mmol) in tetrahydrofuran (150 mL) was added n-butyllithium (34.3 mL, 85.8 mmol) dropwise at −78° C. under N₂ atmosphere. The mixture was stirred at −78° C. for 0.5 hours. To above mixture was added a solution of tert-butyl acetate (9.95 g, 85.8 mmol) in tetrahydrofuran (50 mL) dropwise at −78° C. under N₂ atmosphere. The mixture was stirred at −78° C. for 0.5 hours before a solution of 43-17 (9 g, 33 mmol) in tetrahydrofuran (100 mL) was added dropwise at −78° C. The mixture was stirred at −78° C. for 0.5 hours before it was quenched with sat. NH₄Cl solution and extracted with ethyl acetate. The combined organic layers were washed with water and brine, dried over sodium sulfate, filtered and concentrated. The residue was purified by column chromatography on silica gel (petroleum ether to petroleum ether/dichloromethane=3/1) to afford 43-18.

Step 19: A mixture of 43-18 (12.5 g, 32 mmol) in dioxane (75 mL) and conc. hydrochloride (75 mL) was stirred at 100° C. for 2 hours. The mixture was cooled and poured into water, filtered and the filter cake was washed with water and triturated with acetonitrile to afford 43-19.

Step 20: To a solution of 43-19 (4.9 g, 23 mmol) in N,N-dimethylformamide (60 mL) was added N-chlorosuccinimide (15.3 g, 115 mmol). The mixture was stirred at 100° C. for 3 hours under N₂ atmosphere. Additional N-chlorosuccinimide (15.3 g, 115 mmol) was added and the mixture was stirred at 100° C. for 5 hours. The mixture was cooled, diluted with ethyl acetate, washed with water and brine. The organic layer was dried over sodium sulfate, filtered and concentrated. The residue was triturated with ethyl acetate to afford 43-20.

Step 21: A mixture of 43-20 (1.24 g, 5 mmol), N,N-diisopropylethylamine (1.94 g, 15 mmol), tert-butyl 3,8-diazabicyclo[3.2.1]octane-8-carboxylate (1.59 g, 7.5 mmol) in dimethyl sulfoxide (30 mL) was stirred at 90° C. for 3 hours under nitrogen atmosphere. The mixture was cooled, diluted with ethyl acetate, washed with water and brine. The organic layer was dried over sodium sulfate, filtered and concentrated. The residue was triturated with ethyl acetate to afford 43-21.

Step 22: A mixture of 43-21 (636 mg, 1.5 mmol), 43-7 (477 mg, 3 mmol), 2,2′-bis(diphenylphosphino)-1,1′-binaphthalene (94 mg, 0.15 mmol), sodium tert-butoxide (576 mg, 6 mmol) and tris(dibenzylideneacetone)dipalladium (69 mg, 0.075 mmol) in dioxane (20 mL) was stirred at 110° C. for 3 hours under N₂ atmosphere. The mixture was cooled, diluted with ethyl acetate, washed with water and brine. The organic layer was dried over sodium sulfate, filtered and concentrated. The residue was purified by reverse phase HPLC (acetonitrile with 0.05% of TFA in water: 15% to 95%) to afford 43-22.

Step 23: To a solution of 43-22 (273 mg, 0.5 mmol) in DMF (10 mL) was added cesium carbonate (325 mg, 1 mmol) and N,N-bis(trifluoromethylsulfonyl)aniline (357 mg, 1 mmol) at room temperature. The mixture was stirred at room temperature for 1 hour before it was diluted with ethyl acetate, washed with water and brine. The organic layer was dried over sodium sulfate, filtered and concentrated. The residue was purified by reverse phase HPLC (acetonitrile with 0.05% of TFA in water: 15% to 95%) to afford 43-23.

Step 24: A mixture of 43-23 (102 mg, 0.15 mmol), 43-15 (91 mg, 0.3 mmol), sodium carbonate (64 mg, 0.6 mmol) and tetrakis(triphenylphosphine)palladium (17 mg, 0.015 mmol) in 1,4-dioxane/water (3 mL/0.6 mL) was stirred at 100° C. for 0.3 hour under N₂ atmosphere under microwave condition. The mixture was concentrated and the residue was purified by reverse phase HPLC (acetonitrile with 0.05% of TFA in water: 25% to 95%) to afford 43-24.

Step 25: A solution of 43-24 (20 mg, 0.028 mmol) and trifluoroacetic acid (0.5 mL) in dichloromethane (1.5 mL) was stirred at room temperature for 1 hour. The mixture was concentrated and the residue was purified by prep-HPLC (acetonitrile with 0.05% of TFA in water: 15% to 95%) to afford 43 as a 3 eq of TFA salt. LCMS (ESI, m/z): [M+H]⁺=608.3; ¹H NMR (400 MHz, methanol-d₄, ppm): δ 8.42 (s, 1H), 7.75 (d, J=7.6 Hz, 1H), 7.38-7.29 (m, 3H), 7.02 (d, J=2.0 Hz, 1H), 6.87 (s, 1H), 5.63-5.50 (m, 1H), 4.66-4.53 (m, 2H), 4.22-3.42 (m, 10H), 2.75-2.00 (m, 10H).

### Example 3 Synthesis of Compound 10

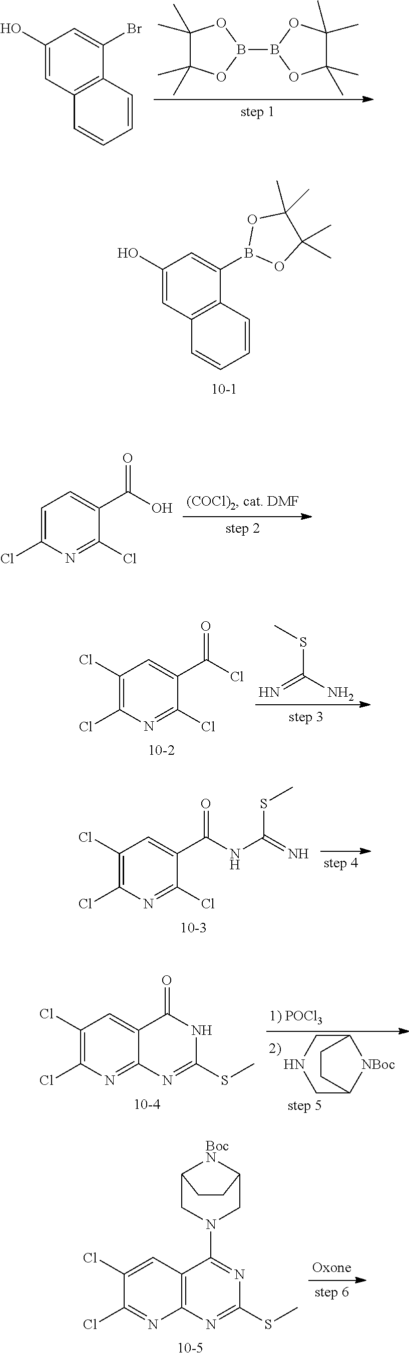

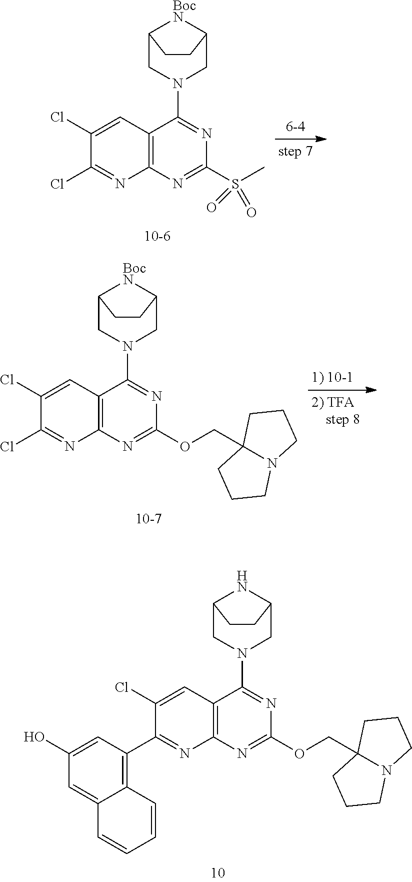 [SMILES: CC(C)(C)OC(=O)N1C2CCC1CN(c1nc(S(C)(=O)=O)nc3nc(Cl)c(Cl)cc13)C2 ; CC(C)(C)OC(=O)N1C2CCC1CN(c1nc(OCC34CCCN3CCC4)nc3nc(Cl)c(Cl)cc13)C2 ; Oc1cc(-c2nc3nc(OCC45CCCN4CCC5)nc(N4CC5CCC(C4)N5)c3cc2Cl)c2ccccc2c1]

Step 1: A mixture of 4-bromonaphthalen-2-ol (3.0 g, 13.4 mmol), bis(pinacolato)diboron (4.1 g, 16.1 mmol), Pd(dppf)Cl₂ (0.98 g, 1.35 mmol) and KOAc (3.9 g, 40.3 mmol) in 1,4-dioxane (30 mL) was stirred at 95° C. for 2 hours under nitrogen atmosphere. The mixture was cooled and diluted with water. The resulting mixture was extracted with ethyl acetate. The combined organic layers were washed with brine, dried over anhydrous Na₂SO₄, filtered and concentrated. The residue was purified by silica gel column chromatography (petroleum ether/ethyl acetate=10/1) to afford 10-1.

Step 2: To a solution of 2,6-dichloronicotinic acid (4.5 g, 20 mmol) in dichloromethane (50 mL) was added a drop of N, N-dimethylformamide and oxalyl chloride (5.0 g, 40 mmol) dropwise at room temperature. The resulting mixture was stirred at 70° C. for 30 min before it was cooled and concentrated to afford 10-2 which was used directly in the next step without purification.

Step 3: To a solution of sodium hydroxide (3.6 g, 90 mmol) in water (30 mL) was added 2-methyl-2-thiopseudourea-sulfate (7 g, 37 mmol) in portions at room temperature followed by addition of a solution of 10-2 (4.9 g, 20 mmol) in tetrahydrofuran (50 mL). The resulting mixture was stirred at room temperature for 30 min before it was diluted with water and extracted with ethyl acetate. The combined organic layers were dried over sodium sulfate, filtered and concentrated. The residue was triturated with hexane to afford 10-3.

Step 4: A solution of 10-3 (4 g, 13.4 mmol) in DMAc (50 mL) was stirred at 100° C. for 24 hours under N₂ atmosphere. The mixture was diluted with water and extracted with ethyl acetate. The combined organic layers were dried over sodium sulfate, filtered and concentrated. The residue was triturated with petroleum ether/ethyl acetate (5/1) and filtered. The filtered cake was dried to afford 10-4.

Step 5: To a solution of 10-4 (400 mg, 1.53 mmol) in acetonitrile (20 mL) were added DIEA (296 mg, 2.3 mmol) and phosphorus oxychloride (285 mg, 1.84 mmol) at room temperature. The resulting mixture was stirred at 80° C. for 1 hour and then cooled to −10° C., DIEA (296 mg, 2.3 mmol) and 8-boc-3,8-diaza-bicyclo[3.2.1]octane (342 mg, 1.53 mmol) were added. The reaction mixture was stirred at room temperature for 1 hour before it was diluted with water and extracted with ethyl acetate. The combined organic layers were dried over sodium sulfate, filtered and concentrated. The residue was purified by column chromatography on silica gel (dichloromethane to dichloromethane/methanol=10/1) to afford 10-5.

Step 6: To a solution of 10-5 (500 mg, 1.13 mmol) in dichloromethane (20 mL) was added oxone (3.3 g, 5.4 mmol). The mixture was stirred at room temperature for 3 d. Then the suspension was filtered and the filtrate was concentrated. The residue was purified by pre-TLC (petroleum ether/ethyl acetate=1/1) to afford 10-6.

Step 7: To a solution of (tetrahydro-1H-pyrrolizin-7a(5H)-yl) methanol (173 mg, 1.23 mmol) in THF (5 mL) was added NaH (60%, 49 mg, 1.23 mmol) at 0° C. under nitrogen. The mixture was stirred at 0° C. for 30 min and then 10-6 (200 mg, 0.40 mmol) was added. The resulting mixture was stirred at room temperature for 1 hour before it was quenched with saturated ammonium chloride (20 mL) and extracted with ethyl acetate. The combined organic layers were dried over sodium sulfate, filtered and concentrated. The residue was purified by column chromatography on silica gel (dichloromethane/methanol=10/1) to afford 10-7.

Step 8: A mixture of 10-7 (10 mg, 0.018 mmol), 4-(4,4,5,5-tetramethyl-1,3,2-dioxaborolan-2-yl)naphthalen-2-ol (10 mg, 0.036 mmol), Na₂CO₃ (8 mg, 0.072 mmol) and Pd(PPh₃)₄ (2 mg, 0.0018 mmol) in 1, 4-dioxane/water (2 mL/0.2 mL) was stirred at 105° C. for 1 hour under microwave condition. The mixture was cooled and trifluoroacetic acid (1 mL) was added. The resulting mixture was stirred at room temperature for 1 hour. The mixture was concentrated and the residue was purified by prep-HPLC (acetonitrile with 0.05% of TFA in water: 5% to 45%) to afford 10 as a 3 eq of TFA salt. LCMS (ESI, m/z): [M+H]⁺=557.3; ¹H NMR (400 MHz, methanol-d₄, ppm): δ 8.62 (s, 1H), 7.43-7.39 (m, 2H), 7.33 (d, J=8.0 Hz, 1H), 7.28 (d, J=2.4 Hz, 1H), 7.22-7.13 (m, 2H), 4.79-4.78 (m, 2H), 4.63 (s, 2H), 4.23 (s, 2H), 3.92 (d, J=14.0 Hz, 2H), 3.66-3.63 (m, 2H), 3.25-3.24 (m, 2H), 2.32-2.06 (m, 12H).

### Example 4 Synthesis of Compound 2

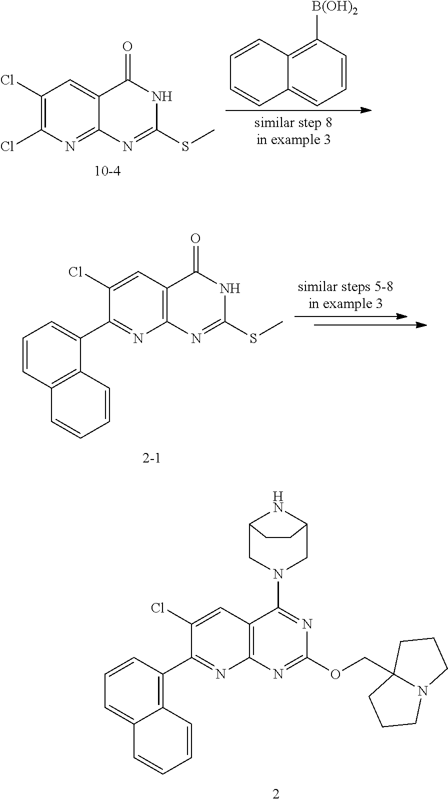

Compound 2-1 was prepared following the coupling procedure for the synthesis of compound 10 in example 3.

Compound 2 was prepared following the procedure for the synthesis of compound 10 in example 3 as a 3 eq of TFA salt. LCMS (ESI, m/z): [M+H]⁺=541.3; ¹H NMR (400 MHz, methanol-d₄, ppm): δ 8.63 (s, 1H), 8.04 (d, J=8.4 Hz, 1H), 7.98 (d, J=8.4 Hz, 1H), 7.65-7.61 (m, 1H), 7.56-7.52 (m, 2H), 7.52-7.44 (m, 2H), 4.79-4.78 (m, 2H), 4.63 (s, 2H), 4.24 (s, 2H), 3.98-3.88 (m, 2H), 3.67-3.61 (m, 2H), 3.25-3.24 (m, 2H), 2.32-2.06 (m, 12H).

### Example 5 Synthesis of Compound 60

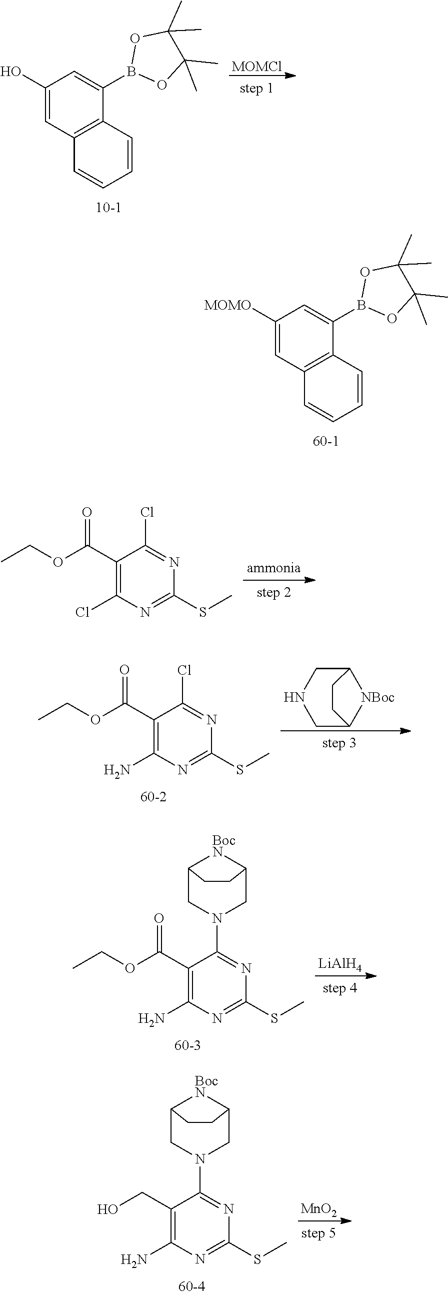

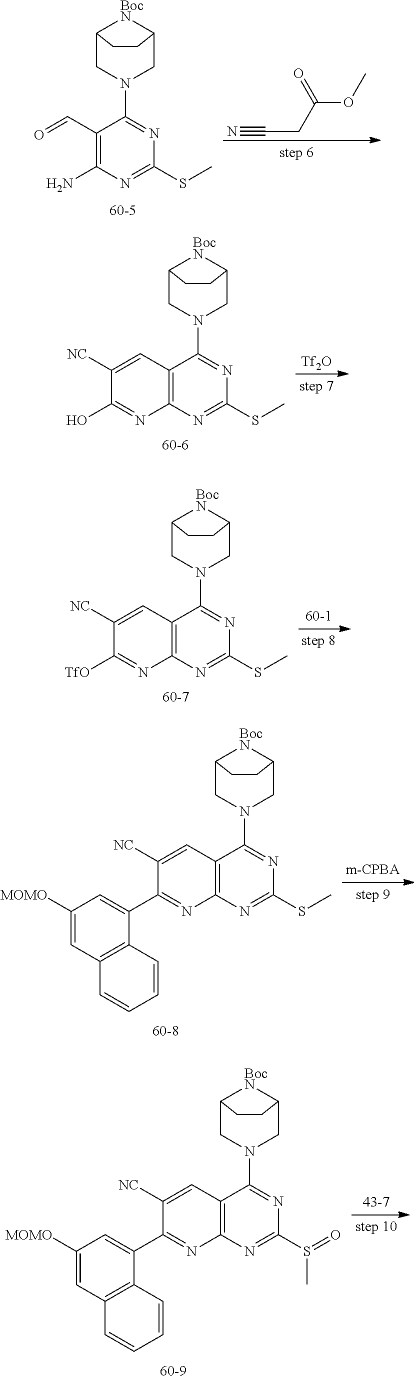

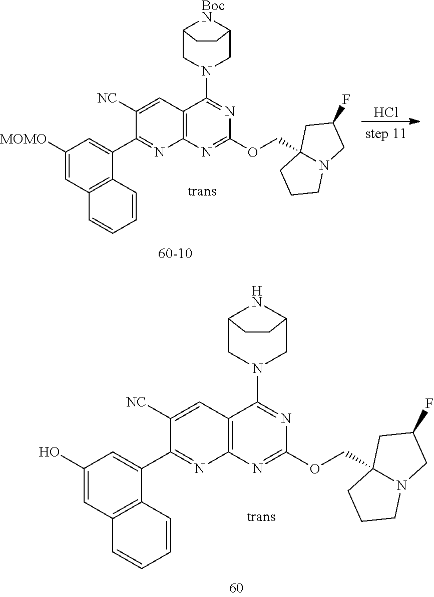

Step 1: A mixture of 10-1 (2.7 g, 10 mmol), N,N-diisopropylethylamine (2.6 g, 20 mmol) and chloro(methoxy)methane (1.21 g, 15 mmol) in dichloromethane (40 mL) was stirred at room temperature overnight. The mixture was diluted with dichloromethane, and washed with water. The organic layer was dried over Na₂SO₄, filtered and concentrated. The residue was purified by column chromatography on silica gel (petroleum ether to petroleum ether/ethyl acetate=9/1) to afford 60-1.

Step 2: To a solution of ethyl 4,6-dichloro-2-(methylthio)pyrimidine-5-carboxylate (5.32 g, 20 mmol) in tetrahydrofuran (50 mL) was added ammonia (28%, 14 mL) at room temperature. The mixture was stirred at room temperature for 4 hours. The mixture was diluted with ethyl acetate and washed with water. The organic layer was dried over sodium sulfate, filtered and concentrated to afford 60-2 which was used directly in the next step without purification.

Step 3: A mixture of 60-2 (3.95 g, 16 mmol), N,N-diisopropylethylamine (3.1 g, 24 mmol), and tert-butyl 3,8-diazabicyclo[3.2.1]octane-8-carboxylate (4.07 g, 19.2 mmol) in dimethyl sulfoxide (20 mL) was stirred at 50° C. for 2 hours under nitrogen atmosphere. The mixture was diluted with ethyl acetate and washed with water. The organic layer was dried over sodium sulfate, filtered and concentrated to afford 60-3 which was used directly in the next step without purification.

Step 4: To a solution of 60-3 (846 mg, 2 mmol) in tetrahydrofuran (20 mL) was added lithium aluminium hydride (228 mg, 6 mmol) in portions at 0° C. under N₂ atmosphere. The mixture was stirred at this temperature for 2 hours. The reaction was quenched with sodium sulfate decahydrate. Then the suspension was filtered and the filtrate was concentrated. The residue was purified by column chromatography on silica gel (petroleum ether to petroleum ether/ethyl acetate=1/2) to afford 60-4.

Step 5: A mixture of 60-4 (534 mg, 1.4 mmol) and manganese dioxide (2.4 g, 28 mmol) in trichloromethane (20 mL) was stirred at 50° C. for 2 hours under nitrogen atmosphere. Then the suspension was filtered and washed with ethyl acetate. The filtrate was concentrated and the residue was purified by column chromatography on silica gel (petroleum ether to petroleum ether/ethyl acetate=1/1) to afford 60-5.

Step 6: To a solution of 60-5 (417 mg, 1.1 mmol) in ethanol (10 mL) was added piperidine (187 mg, 2.2 mmol) and methyl cyanoacetate (163 mg, 1.65 mmol) at room temperature. The mixture was stirred at reflux for 16 hours. The mixture was concentrated and the residue was purified by reverse phase HPLC (acetonitrile with 0.05% of TFA in water: 25% to 95%) to afford 60-6.

Step 7: To a solution of 60-6 (342 mg, 0.8 mmol) and triethylamine (162 mg, 1.6 mmol) in dichloromethane (10 mL) was added trifluoromethanesulfonic anhydride (338 mg, 1.2 mmol) at 0° C. The mixture was stirred at 0° C. for 1 hour. The mixture was diluted with ethyl acetate and washed with water. The organic layer was dried over sodium sulfate, filtered and concentrated. The residue was purified by column chromatography on silica gel (petroleum ether to petroleum ether/ethyl acetate=1/1) to afford 60-7.

Step 8: A mixture of 60-7 (280 mg, 0.5 mmol), 60-1 (188 mg, 0.6 mmol), sodium carbonate (212 mg, 2 mmol) and tetrakis(triphenylphosphine)palladium (58 mg, 0.05 mmol) in 1,4-dioxane/water (5/1, 6 mL) was stirred at 95° C. for 30 min under microwave condition. The mixture was cooled, diluted with ethyl acetate and washed with water. The organic layer was dried over sodium sulfate, filtered and concentrated. The residue was purified by column chromatography on silica gel (petroleum ether to petroleum ether/ethyl acetate=1/2) to afford 60-8.

Step 9: To a solution of 60-8 (150 mg, 0.25 mmol) in dichloromethane (10 mL) was added 3-chloroperbenzoic acid (51 mg, 0.25 mmol) at room temperature. The mixture was stirred at room temperature for 1 hour before it was diluted with ethyl acetate and washed with saturated aqueous sodium bicarbonate solution and brine. The organic layer was dried over sodium sulfate, filtered and concentrated to afford 60-9 which was used directly in the next step without purification.

Step 10: 60-9 that was obtained in the previous step was dissolved in anhydrous tetrahydrofuran (10 mL) and treated with 43-7 (119 mg, 0.75 mmol). To this mixture was added dropwise lithium bis(trimethylsilyl)amide (0.5 mL, 0.5 mmol, 1 M in tetrahydrofuran) at 0° C. under N₂ atmosphere before the reaction was stirred at 0° C. for 30 min. The mixture was diluted with ethyl acetate and washed with water. The organic layer was dried over sodium sulfate, filtered and concentrated. The residue was purified by reverse phase HPLC (acetonitrile with 0.05% of TFA in water: 20% to 95%) to afford 60-10.

Step 11: To a solution of 60-10 (46 mg, 0.065 mmol) in 1,4-dioxane (0.8 mL) was added water (0.4 mL) and concentrated hydrochloric acid (0.4 mL). The mixture was stirred at room temperature for 2 hours. The mixture was purified by prep-HPLC (acetonitrile with 0.05% of TFA in water: 5% to 95%) to afford 60 as a 3 eq of TFA salt. LCMS (ESI, m/z): [M+H]⁺=566.3; ¹H NMR (400 MHz, methanol-d₄, ppm): δ 8.98 (s, 1H), 7.79-7.76 (m, 1H), 7.59-7.56 (m, 1H), 7.47-7.43 (m, 1H), 7.34-7.33 (m, 1H), 7.29-7.23 (m, 2H), 5.63-5.49 (m, 1H), 4.89-4.83 (m, 2H), 4.73-4.65 (m, 2H), 4.24 (s, 2H), 4.02-3.85 (m, 5H), 3.49-3.42 (m, 1H), 2.74-2.03 (m, 10H). ¹⁹F NMR (376 MHz, methanol-d₄, ppm): δ −174.24 (1F).

### Example 6 Synthesis of Compound 6

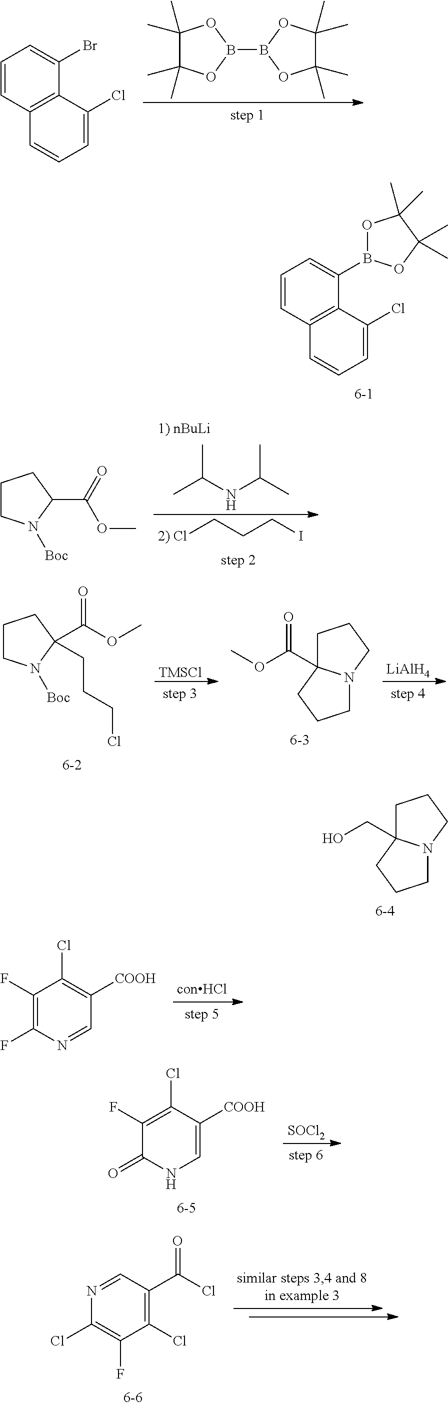

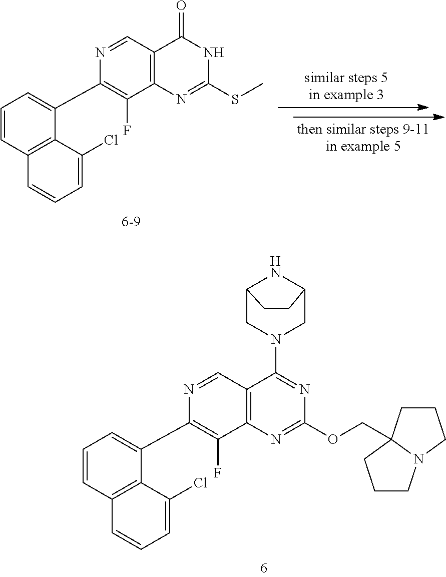 [SMILES: CSc1nc2c(F)c(-c3cccc4cccc(Cl)c34)ncc2c(=O)[nH]1 ; Fc1c(-c2cccc3cccc(Cl)c23)ncc2c(N3CC4CCC(C3)N4)nc(OCC34CCCN3CCC4)nc12]

Step 1: A mixture of 1-bromo-8-chloronaphthalene (5.0 g, 20.7 mmol) and bis(pinacolato)diboron (5.8 g, 22.8 mmol), Pd(dppf)Cl₂ (1.5 g, 2.1 mmol) and KOAc (6.1 g, 62.1 mmol) in DMF (120 mL) was stirred at 80° C. for 3 hours under nitrogen atmosphere. The mixture was cooled and diluted with water. The resulting mixture was extracted with ethyl acetate. The combined organic layers were washed with brine, dried over anhydrous Na₂SO₄ and filtered. The residue was purified by silica gel column chromatography (petroleum ether/ethyl acetate=20/1) to afford 6-1.

Step 2: To a solution of di-isopropylamine (37.1 g, 366.4 mmol) in THF was added n-BuLi (2.5 M in hexane, 136.0 mL, 340.2 mmol) dropwise at −78° C. under argon atmosphere. The mixture was stirred at −78° C. for 20 min, followed by addition of 1-tert-butyl 2-methyl pyrrolidine-1,2-dicarboxylate (60.0 g, 261.7 mmol) in THF. The resulting mixture was stirred at −78° C. for 1 hour before addition of 1-chloro-3-iodopropane (107.0 g, 523.4 mmol) dropwise. The resulting mixture was stirred overnight at room temperature and then quenched with sat. NH₄Cl (aq.). The aqueous layer was extracted with ethyl acetate. The combined organic layers were dried over anhydrous Na₂SO₄, filtered and concentrated. The residue was purified by flash column chromatography on silica gel (petroleum ether/ethyl acetate=10/1) to afford 6-2.

Step 3: To a solution of 6-2 (69.0 g, 225.6 mmol) in methanol (1.4 L) was added TMSCl (122.6 g, 1128.2 mmol) at 0° C. The mixture was stirred overnight at room temperature. The mixture was basified to pH 8 with sat. NaHCO₃ solution. The aqueous layer was extracted with dichloromethane. The combined organic layers were dried over anhydrous Na₂SO₄, filtered and concentrated. The residue was purified by flash column chromatography on silica gel (dichloromethane/methanol=10/1) to afford 6-3.

Step 4: To a solution of 6-3 (20.0 g, 118.2 mmol) in THF (200 mL) was added LiAlH₄ (6.7 g, 177.3 mmol) in portions at 0° C. under nitrogen atmosphere. The resulting mixture was stirred at 0° C. for 30 min. The reaction was quenched by Na₂SO₄.10H₂O (20 g) and then 15% NaOH (5 mL) at 0° C. Then the suspension was filtered and washed with THF. The combined organic layers were dried over anhydrous Na₂SO₄, filtered and concentrated to afford 6-4.

Step 5: To a solution of 4-chloro-5,6-difluoropyridine-3-carboxylic acid (14.1 g, 73.2 mmol) in 1,4-dioxane/water (30 mL/30 mL) was added conc. HCl (60 mL). The resulting mixture was stirred at 100° C. for 2 hours under N₂ atmosphere vigorously. The mixture was cooled and filtered. The filtrate cake was collected and triturated with acetonitrile to afford 6-5.

Step 6: To a suspension of 6-5 (10.05 g, 52.6 mmol) in thionyl chloride (100 mL) was added N,N-dimethylformamide (576 mg, 7.9 mmol). The resulting mixture was stirred at 85° C. for 1.5 hours. The mixture was cooled and concentrated to afford 6-6 which is used directly in the next step without purification.

Compound 6-9 was prepared from 6-6 following the coupling procedure for the synthesis of compound 10 in example 3.

Compound 6 was prepared from 6-9 following the procedure for the synthesis of compound 10-5 in example 3 and compound 60 in example 5 as a 3 eq of TFA salt. LCMS (ESI, m/z): [M+H]⁺=559.3; ¹H NMR (400 MHz, methanol-d₄, ppm): δ 9.11 (s, 1H), 8.13 (d, J=8.0 Hz, 1H), 8.00 (d, J=8.0 Hz, 1H), 7.68 (t, J=8.0 Hz, 1H), 7.59-7.57 (m, 2H), 7.50 (t, J=8.0 Hz, 1H), 4.84-4.80 (m, 2H), 4.66 (s, 2H), 4.28-4.22 (m, 2H), 4.01-3.94 (m, 2H), 3.72-3.66 (m, 2H), 3.27-3.24 (m, 2H), 2.34-2.07 (m, 12H). ¹⁹F NMR (376 MHz, methanol-d₄, ppm): δ −139.26 (1F).

### Example 7 Synthesis of Compound 47

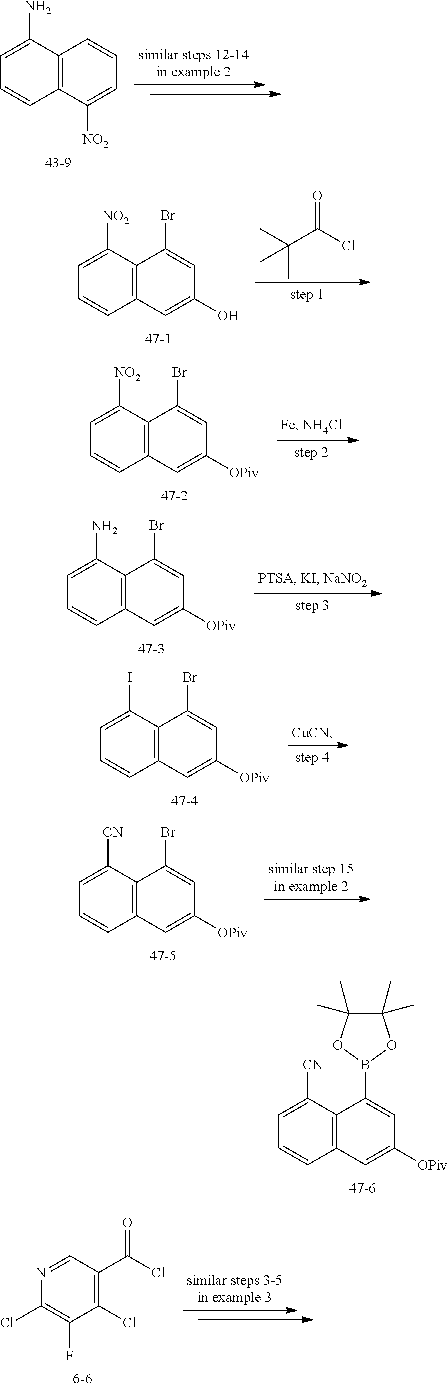

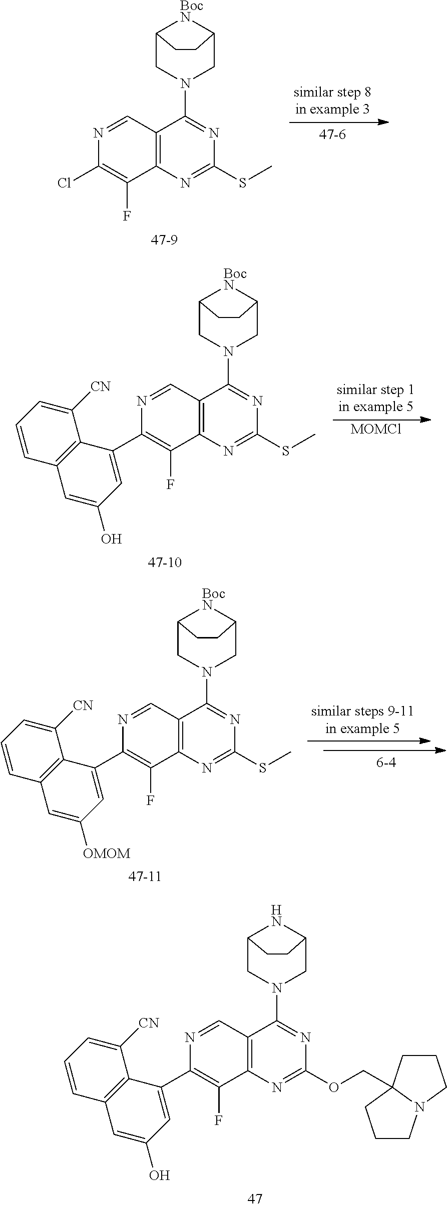

Compound 47-1 was prepared from 43-9 following the procedure for the synthesis of compound 43-14 in example 2.

Step 1: To a solution of 47-1 (10.7 g, 40 mmol) and triethylamine (6.06 g, 60 mmol) in dichloromethane (100 mL) was added pivaloyl chloride (5.76 g, 48 mmol) dropwise at 0° C. The mixture was stirred for 1 hour at room temperature. The result mixture was washed with water and brine. The organic layer was dried over sodium sulfate, filtered and concentrated to afford 47-2 which was used directly in the next step without purification.

Step 2: A mixture of 47-2 (8.1 g, 23 mmol), iron powder (6.5 g, 115 mmol) and ammonium chloride (12.2 g, 230 mmol) in ethanol (40 mL) and water (10 mL) was stirred at 80° C. for 10 min under N₂ atmosphere. The mixture was cooled, diluted with water and extracted with ethyl acetate. The combined organic layers were dried over sodium sulfate, filtered and concentrated. The residue was purified by silica gel column chromatograph (petroleum ether to petroleum ether/ethyl acetate=4/1) to afford 47-3.

Step 3: To a solution of 47-3 (5.06 g, 15.76 mmol) in acetonitrile (126 mL) was added p-toluenesulfonic acid (8.13 g, 47.29 mmol). The mixture was stirred at room temperature for 30 min. To above mixture was added a solution of sodium nitrite (2.17 g, 31.52 mmol) and potassium iodide (5.23 g, 31.52 mmol) in water (19 mL) at 0° C. over 30 min. The resulting mixture was allowed to warm to 30° C. and stirred for 2 hours. The mixture was diluted with dichloromethane, washed with water, saturated aqueous sodium bicarbonate solution and brine successively. The organic layer was dried over sodium sulfate, filtered and concentrated. The residue was purified by silica gel column chromatograph (petroleum ether to petroleum ether/ethyl acetate=10/1) to afford 47-4.

Step 4: A mixture of 47-4 (3.4 g, 7.87 mmol) and copper (I) cyanide (744 mg, 8.26 mmol) in N,N-dimethylformamide (34 mL) was stirred at 80° C. for 0.5 hours under N₂ atmosphere. The organic layer was cooled, diluted with ethyl acetate and filtered. The organic layer was washed with water and brine, dried over sodium sulfate, filtered and concentrated. The residue was triturated with n-hexane to afford 47-5.

Compound 47-6 was prepared from 47-5 following the procedure for the synthesis of compound 43-15 in example 2.

Compound 47-9 was prepared from 6-6 following the procedure for the synthesis of compound 10-5 in example 3.

Compound 47-10 was prepared from 47-6 and 47-9 following the coupling procedure for the synthesis of compound 10 in example 3.

Compound 47-11 was prepared from 47-10 following the procedure for the synthesis of compound 60-1 in example 5.

Compound 47 was prepared from 47-11 following the procedure for the synthesis of compound 60 in example 5 as a 3 eq of TFA salt. LCMS (ESI, m/z): [M+H]⁺=566.3; ¹H NMR (400 MHz, methanol-d₄, ppm): δ 9.13 (s, 1H), 8.12-8.10 (m, 1H), 7.75 (dd, J=7.2, 0.8 Hz, 1H), 7.54 (t, J=8.4 Hz, 1H), 7.43-7.42 (m, 1H), 7.35-7.34 (m, 1H), 4.95-4.85 (m, 2H), 4.68 (s, 2H), 4.28-4.22 (m, 2H), 4.07-3.90 (m, 2H), 3.74-3.63 (m, 2H), 3.31-3.25 (m, 2H), 2.38-2.29 (m, 2H), 2.28-2.01 (m, 10H).

### Example 8 Synthesis of Compound 56

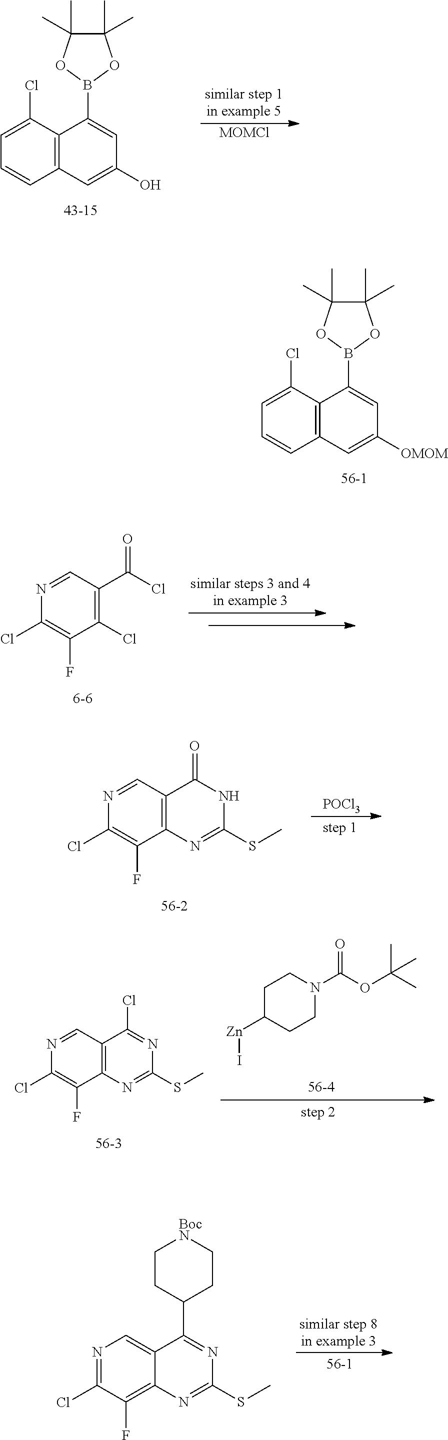

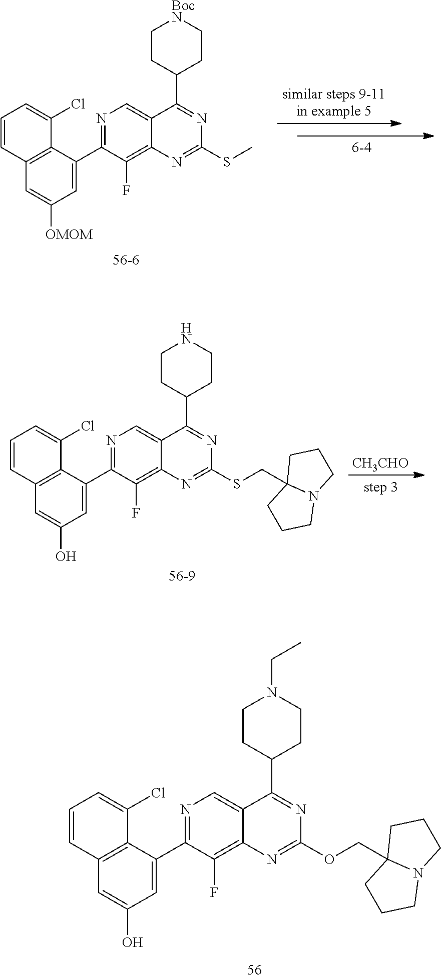

Compound 56-1 was prepare rom 43-15 following the procedure for the synthesis of compound 60-1 in example 5.

Compound 56-2 was prepared from 6-6 following the procedure for the synthesis of compound 10-4 in example 3.

Step 1: To a suspension of 56-2 (2.45 g, 10 mmol) in acetonitrile (100 mL) was added N,N-diisopropylethylamine (1.94 g, 15 mmol) and phosphorus oxychloride (1.84 g, 12 mmol) at room temperature. The mixture was stirred at 80° C. for 1 hour under N₂ atmosphere. The mixture was cooled and partitioned between ethyl acetate and water. The organic layer was washed with saturated aqueous sodium bicarbonate solution, brine, dried over sodium sulfate, filtered and concentrated. The residue was triturated with petroleum ether to afford 56-3.

Step 2: To a mixture of N-Boc-4-iodopiperidine (3.11 g, 10 mmol) and zinc dust (780 mg, 12 mmol) in tetrahydrofuran (20 mL) was added chlorotrimethylsilane (109 mg, 0.1 mmol). The mixture was stirred at 40° C. for 1 hour and then allowed to cool to room temperature to obtain a solution of 56-4. To a mixture of 56-2 (1.4 g, 5.3 mmol), [1,1′-bis(diphenylphosphino)ferrocene]dichloropalladium(II) (194 mg, 0.265 mmol), cuprous iodide (101 mg, 0.53 mmol) in N,N-dimethylacetamide (14 mL) was added the freshly prepared zinc reagent 56-4. The resulting mixture was stirred at 80° C. for 6 hours under N₂ atmosphere. The mixture was cooled, diluted with ethyl acetate, washed with water and brine. The organic layer was dried over sodium sulfate, filtered and concentrated. The residue was purified by reverse phase HPLC (acetonitrile with 0.05% of TFA in water: 15% to 95%) to afford 56-5 which was used directly in the next step.

Compound 56-6 was prepared from 56-5 following the coupling procedure for the synthesis of compound 10 in example 3.

Compound 56-9 was prepared from 56-6 following the procedure for the synthesis of compound 60 in example 5.

Step 3: To a suspension of 56-9 (11 mg, 0.02 mmol) in acetic acid (1.2 mg, 0.02 mmol) and 1,2-dichloroethane (2 mL) was added acetaldehyde (0.04 mL, 0.2 mmol, 5 M in tetrahydrofuran) followed by sodium triacetoxyborohydride (21.2 mg, 0.1 mmol) at room temperature. When the reaction was complete as judged by TLC, it was quenched with saturated aqueous sodium bicarbonate solution and concentrated. The residue was purified by prep-HPLC (acetonitrile with 0.05% of TFA in water: 5% to 95%) to afford 56 as a 3 eq of TFA salt. LCMS (ESI, m/z): [M+H]⁺=576.3; ¹H NMR (400 MHz, methanol-d₄, ppm): δ 9.50 (s, 1H), 7.76 (d, J=8.0 Hz, 1H), 7.39-7.31 (m, 3H), 7.18-7.14 (m, 1H), 4.76 (s, 2H), 4.26-4.16 (m, 1H), 3.78-3.66 (m, 4H), 3.33-3.21 (m, 4H), 2.50-2.09 (m, 14H), 1.40 (t, J=7.2 Hz, 3H).

### Example 9 Synthesis of Compound 73

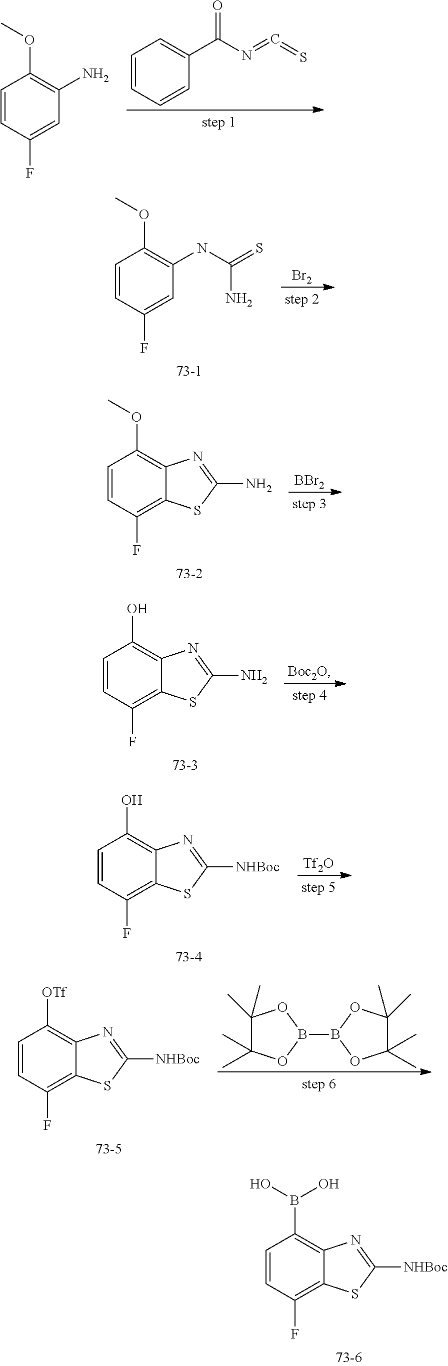

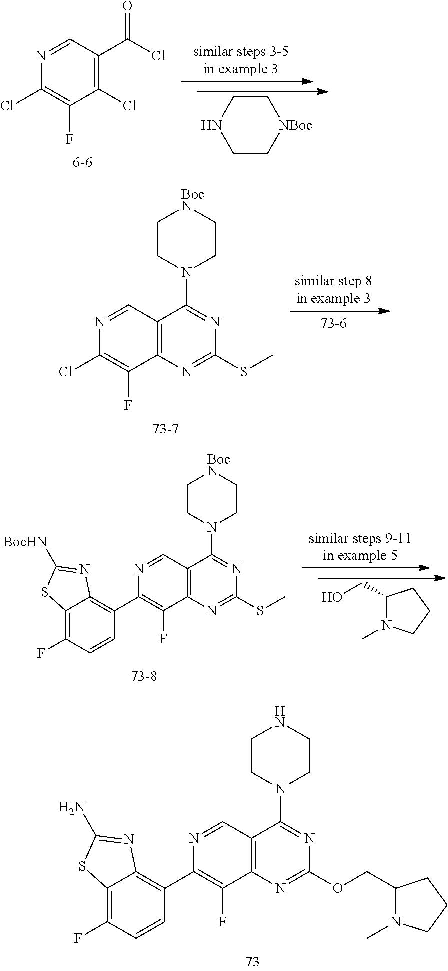

Step 1: To a solution of benzoylisothiocyanate (36.4 g, 223.2 mmol) in anhydrous THF (150 mL) was added a solution of 5-fluoro-2-methoxy-aniline (30.0 g, 212.5 mmol) in anhydrous THE (150 mL) at 0° C. under nitrogen atmosphere. After addition, the mixture was allowed to warm to room temperature and stirred for 3 hours. Then NaOH (1 M, 216.8 mL) solution was added and the resulting mixture was stirred at 80° C. overnight. The mixture was concentrated and filtered. The filter cake was washed with cold hexane to afford 73-1 which was used directly in the next step without purification.

Step 2: To a solution of 73-1 (43.0 g, 214.7 mmol) in CHCl₃ (900 mL) was added Br₂ (35.0 g, 219.1 mmol) dropwise at 0° C. After being stirred at 0° C. for 0.5 hours, the mixture was heated at reflux for 2 hours. Then the reaction mixture was cooled, filtered. The filter cake was washed with cold hexane to afford 73-2 which was used directly in the next step without purification.

Step 3: To a solution of 73-2 (20.0 g, 100.9 mmol) in dichloromethane was added BBr₃ (1 M in dichloromethane, 312.8 mL) dropwise at 0° C. The mixture was warmed to room temperature and stirred overnight. The reaction was quenched with methanol at 0° C. Then the suspension was filtered and the filter cake was washed with cold dichloromethane to afford 73-3 which was used directly in the next step without purification.

Step 4: To a mixture of 73-3 (16.8 g, 91.2 mmol), Et₃N (19.4 g, 191.5 mmol) and DMAP (557.2 mg, 4.6 mmol) in dichloromethane (280 mL) was added Boc₂O (45.8 g, 209.8 mmol) at room temperature. The mixture was stirred at room temperature overnight before it was diluted with water and extracted with ethyl acetate. The organic layer was concentrated and re-dissolved in methanol (180 mL). MeONa (5.4 M in methanol, 25 mL) was added and the mixture was stirred at room temperature overnight. The mixture was diluted with water and extracted with ethyl acetate. The combined organic layers were washed with brine, dried over sodium sulfate, filtered and concentrated to afford 73-4 which was used directly in the next step without purification.

Step 5: To a solution of 73-4 (23.0 g, 80.9 mmol) and pyridine (12.8 g, 161.8 mmol, 13.0 mL) in dichloromethane (60 mL) was added Tf₂O (27.4 g, 97.1 mmol) at 0° C. The mixture was stirred at 0° C. for 1 hour before it was diluted with water and extracted with dichloromethane. The combined organic layers were washed with brine, dried over sodium sulfate, filtered and concentrated. The residue was purified by column chromatography on silica gel (petroleum ether/ethyl acetate=20/1) to afford 73-5.

Step 6: A mixture of 73-5 (18.0 g, 43.2 mmol), 4,4,5,5-tetramethyl-2-(4,4,5,5-tetramethyl-1,3,2-dioxaborolan-2-yl)-1,3,2-dioxaborolane (87.8 g, 345.8 mmol), KOAc (12.7 g, 129.7 mmol) and Pd(PPh₃)₄ (10.0 g, 8.65 mmol) in 1,4-dioxane (240 mL) was stirred at 80° C. overnight. The mixture was diluted with water and extracted with ethyl acetate. The combined organic layers were washed with brine, dried over sodium sulfate, filtered and concentrated. The residue was purified by reverse phase HPLC (acetonitrile with 0.05% of TFA in water: 10% to 95%) to afford 73-6.

Compound 73-7 was prepared from 6-6 following the procedure for the synthesis of compound 10-5 in example 3.

Compound 73-8 was prepared from 73-7 following the coupling procedure for the synthesis of compound 10 in example 3.

Compound 73 was prepared from 73-8 following the procedure for the synthesis of compound 60 in example 5 as a 3 eq of TFA salt. LCMS (ESI, m/z): [M+H]⁺=513.2; ¹H NMR (400 MHz, methanol-d₄, ppm): δ 9.19 (s, 1H), 7.69 (dd, J=8.8, 5.6 Hz, 1H), 7.11 (t, J=8.8 Hz, 1H), 4.94-4.90 (m, 1H), 4.73-4.69 (m, 1H), 4.29-4.23 (m, 4H), 3.92-3.89 (m, 1H), 3.77-3.72 (m, 1H), 3.52-3.44 (m, 4H), 3.25-3.21 (m, 1H), 3.09 (s, 3H), 2.44-2.36 (m, 1H), 2.24-2.15 (m, 1H), 2.13-2.02 (m, 2H). ¹⁹F NMR (376 MHz, methanol-d₄, ppm): δ −113.56 (1F), −138.21 (1F).

### Example 10 Synthesis of Compound 76

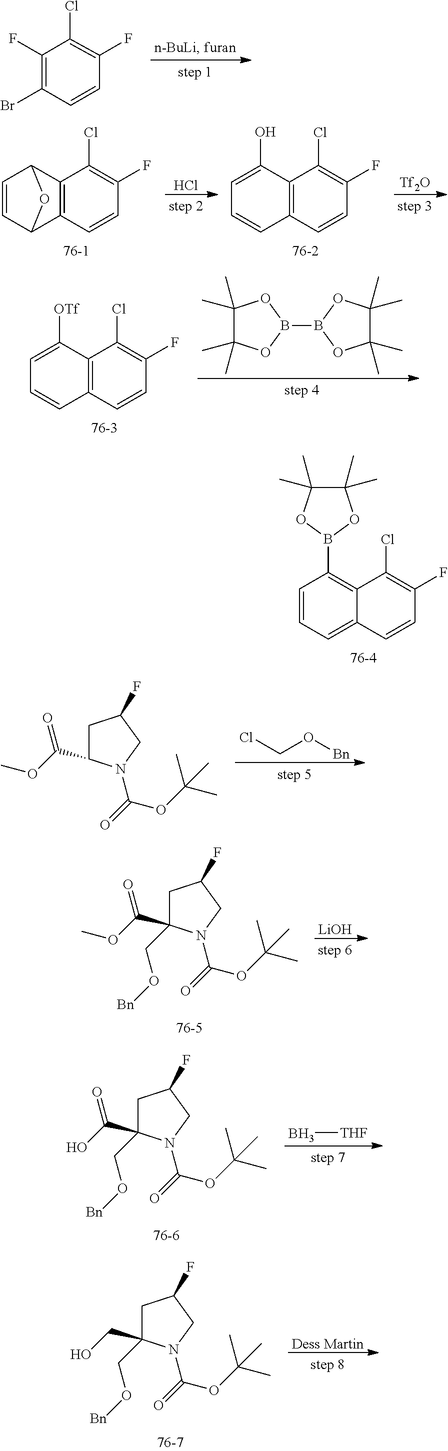

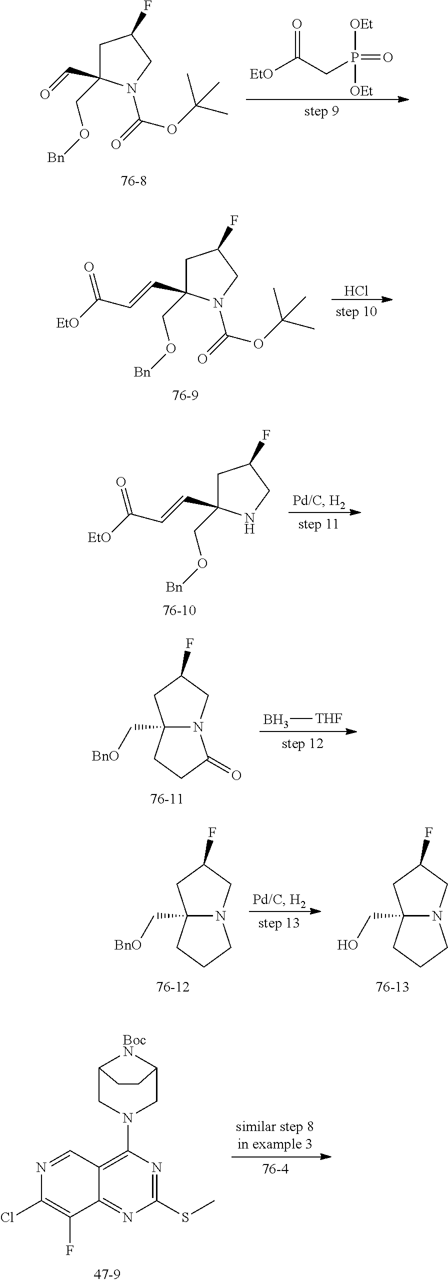

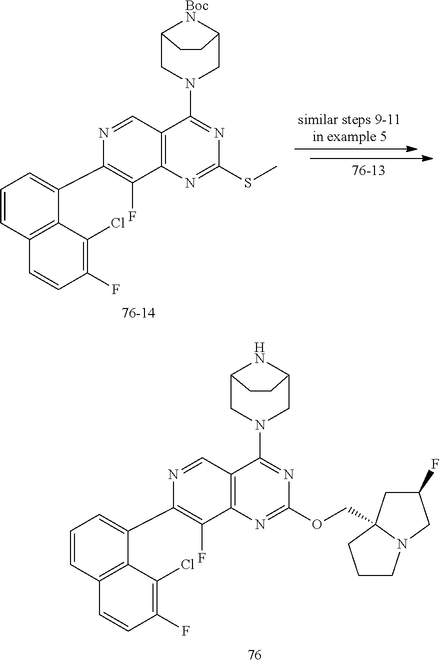 [SMILES: CSc1nc(N2CC3CCC(C2)N3C(=O)OC(C)(C)C)c2cnc(-c3cccc4ccc(F)c(Cl)c34)c(F)c2n1 ; Fc1ccc2cccc(-c3ncc4c(N5CC6CCC(C5)N6)nc(OC[C@@]56CCCN5C[C@H](F)C6)nc4c3F)c2c1Cl]

Step 1: To a mixture of 1-bromo-3-chloro-2,4-difluorobenzene (11.35 g, 50 mmol) and furan (6.8 g, 100 mmol) in toluene (200 mL) was added n-butyllithium (38 mL, 60 mmol, 1.6 M in hexane) dropwise at −15° C. over 0.5 hours under nitrogen atmosphere. The mixture was warmed to room temperature and stirred for 16 hours. The reaction mixture was quenched with water and filtered. The aqueous layer was extracted with ethyl acetate. The combined organic layers were washed with brine, dried over Na₂SO₄, filtered and concentrated. The residue was purified by reverse phase HPLC (acetonitrile with 0.1% of FA in water: 10% to 95%) to afford 76-1.

Step 2: A solution of 76-1 (3.5 g, 17.8 mmol) in conc. HCl (500 mL) and ethanol (40 mL) was stirred at 80° C. for 2 hours. The mixture was concentrated and purified by column chromatography on silica gel (petroleum ether to petroleum ether/ethyl acetate=50/1) to afford 76-2.

Step 3: A mixture of 76-2 (1.2 g, 6.1 mmol), N, N-diisopropylethylamine (3.93 g, 30.5 mmol) and 4 Å molecular sieves (1.2 g) in dichloromethane (25 mL) was stirred for 10 min at room temperature under nitrogen atmosphere. Then trifluoroacetic anhydride (2.1 g, 7.3 mmol) was added at −40° C. and the mixture was stirred at −40° C. for 10 min. The reaction mixture was quenched with water and filtered. The aqueous layer was extracted with dichloromethane. The combined organic layers were washed with brine, dried over sodium sulfate, filtered and concentrated. The residue was purified by column chromatography on silica gel (petroleum ether to petroleum ether/ethyl acetate=50/1) to afford 76-3.

Step 4: A mixture of 76-3 (1.9 g, 5.8 mmol), bis(pinacolato)diboron (2.2 g, 8.7 mmol), potassium acetate (2.26 g, 23 mmol) and [1,1′-bis(diphenylphosphino)ferrocene]dichloropalladium (II) (844 mg, 1.15 mmol) in dimethyl sulfoxide (40 mL) was stirred at 80° C. for 2 hours. Then cooled, filtered, diluted with water and extracted with ethyl acetate. The combined organic layers were washed with brine, dried over sodium sulfate, filtered and concentrated. The residue was purified by reverse phase HPLC (acetonitrile with 0.05% of TFA in water: 10% to 95%) to afford 76-4.

Step 5: To a solution of 1-(tert-butyl) 2-methyl (2S,4R)-4-fluoropyrrolidine-1,2-dicarboxylate (247 g, 1 mol) in tetrahydrofuran (2 L) was added dropwise lithium bis(trimethylsilyl)amide (1.2 L, 1.2 mol, 1.0 M in tetrahydrofuran) at −70° C. under nitrogen atmosphere. The mixture was stirred at −70° C. for 1 hour before a solution of ((chloromethoxy)methyl)benzene (172 g, 1.1 mol) in tetrahydrofuran (300 mL) was added dropwise at −70° C. The mixture was stirred at −30° C. for 5 hours, quenched with sat. aqueous ammonium chloride and extracted with ethyl acetate. The combined organic layers were washed with brine, dried over sodium sulfate, filtered and concentrated to afford 76-5 which was used in the next step directly without purification.

Step 6: To a solution of 76-5 (367 g, 1 mol) in tetrahydrofuran (2 L) and water (600 mL) was added lithium hydroxide monohydrate (114 g, 3 mol) at room temperature. The mixture was stirred at 60° C. overnight. The mixture was concentrated, diluted with water and tert-butyl methyl ether. After being stirred for 30 min, the aqueous phase was separated, adjusted to around pH 3 with 1 N HCl and extracted with ethyl acetate. The combined organic layers were washed with brine, dried over sodium sulfate, filtered and concentrated to afford 76-6 which was used in the next step directly without purification.

Step 7: To a solution of 76-6 (320 g, 906 mmol) in tetrahydrofuran (2.5 L) was added borane tetrahydrofuran complex solution (1.36 L, 1.36 mol, 1.0 M in tetrahydrofuran) dropwise at 0° C. under nitrogen atmosphere. The mixture was stirred at room temperature for 4 hours, quenched with methanol (500 mL) and stirred at reflux for 3 hours. Then the mixture was diluted with water and extracted with ethyl acetate. The combined organic layers were washed with brine, dried over sodium sulfate, filtered and concentrated to afford 76-7 which was used in the next step directly without purification.

Step 8: To a solution of 76-7 (285 g, 840 mmol) in dichloromethane (3500 mL) was added Dess Martin periodinane (445 g, 1050 mmol) at 0° C. The mixture was stirred at room temperature overnight, quenched with sat. aqueous sodium hyposulfite solution and stirred at room temperature for 3 hours. Then, the mixture was filtered and the aqueous layer was extracted with dichloromethane. The combined organic layers were washed with sat. aqueous sodium bicarbonate, brine, dried over sodium sulfate, filtered and concentrated to afford 76-8 which was used in the next step directly without purification.

Step 9: To a solution of ethyl 2-(diethoxyphosphoryl)acetate (211 g, 944 mmol) in tetrahydrofuran (1500 mL) was added dropwise lithium bis(trimethylsilyl)amide (944 mL, 944 mmol, 1.0 M in tetrahydrofuran) at −40° C. under nitrogen atmosphere. The mixture was stirred at −40° C. for 1 hour. Then a solution of 76-8 (265 g, 786 mmol) in tetrahydrofuran (500 mL) was added dropwise to the reaction mixture at −40° C. The resulting mixture was stirred at room temperature for 3 hours, quenched with sat. aqueous ammonium chloride and extracted with ethyl acetate. The combined organic layers were washed with brine, dried over sodium sulfate, filtered and concentrated to afford 76-9 which was used in the next step without purification.

Step 10: To a solution of 76-9 (320 g, 786 mmol) in ethyl acetate (500 mL) was added hydrochloric acid (800 mL, 2.8 mol, 3.5M in ethyl acetate) at room temperature. After being stirred at room temperature for 3 hours, the mixture was concentrated, diluted with water and tert-butyl methyl ether. The mixture was stirred at room temperature for 30 min. The aqueous phase was separated, adjusted to around pH 10 with sat. aqueous sodium carbonate solution and extracted with ethyl acetate. The combined organic layers were washed with brine, dried over sodium sulfate, filtered and concentrated to afford 76-10 which was used in the next step without purification.

Step 11: A mixture of 76-10 (225 g, 733 mmol) and 10% Pd/C (11 g) in ethyl acetate (1.2 L) was stirred at room temperature overnight under hydrogen atmosphere, then heated to reflux and stirred overnight. The mixture was cooled, filtered, and the filtrate was concentrated. The residue was purified by column chromatography on silica gel (petroleum ether to petroleum ether/ethyl acetate=1/4) to afford 76-11.

Step 12: To a solution of 76-11 (130 g, 494 mmol) in tetrahydrofuran (1.5 L) was added borane tetrahydrofuran complex solution (740 mL, 740 mmol, 1.0 M in tetrahydrofuran) dropwise at 0° C. under nitrogen atmosphere. Then the mixture was stirred at room temperature for 4 hours, quenched with methanol and stirred at reflux for 3 hours. The mixture was cooled, diluted with water and extracted with ethyl acetate. The combined organic layers were washed with brine, dried over sodium sulfate, filtered and concentrated to afford 76-12 which was used in the next step without purification.

Step 13: A mixture of 76-12 (2.5 g, 10 mmol) and 10% Pd/C (200 mg) in methanol (30 mL) was stirred at 45° C. overnight under hydrogen atmosphere. Then, the mixture was filtered and the filtrate was concentrated. The residue was purified by column chromatography on silica gel (dichloromethane to dichloromethane/methanol=10/1) to afford 76-13.

Compound 76-14 was prepared from 47-9 and 76-4 following the coupling procedure for the synthesis of compound 10 in example 3.

Compound 76 was prepared from 76-14 following the procedure for the synthesis of compound 60 in example 5 as a 3 eq of TFA salt. LCMS (ESI, m/z): [M+H]⁺=595.3; ¹H NMR (400 MHz, methanol-d₄, ppm): δ 9.13 (s, 1H), 8.16 (dd, J=8.0, 1.2 Hz, 1H), 8.08 (dd, J=9.2, 5.6 Hz, 1H), 7.70-7.62 (m, 2H), 7.53 (t, J=8.8 Hz, 1H), 5.64-5.49 (m, 1H), 4.93-4.89 (m, 2H), 4.73-4.66 (m, 2H), 4.32-4.27 (m, 2H), 4.05-3.83 (m, 5H), 3.49-3.42 (m, 1H), 2.76-2.53 (m, 2H), 2.45-2.30 (m, 3H), 2.22-2.07 (m, 5H). ¹⁹F NMR (376 MHz, methanol-d₄, ppm): δ −111.42 (1F), −139.45 (1F), −174.22 (1F).

### Example 11 Synthesis of Compound 17

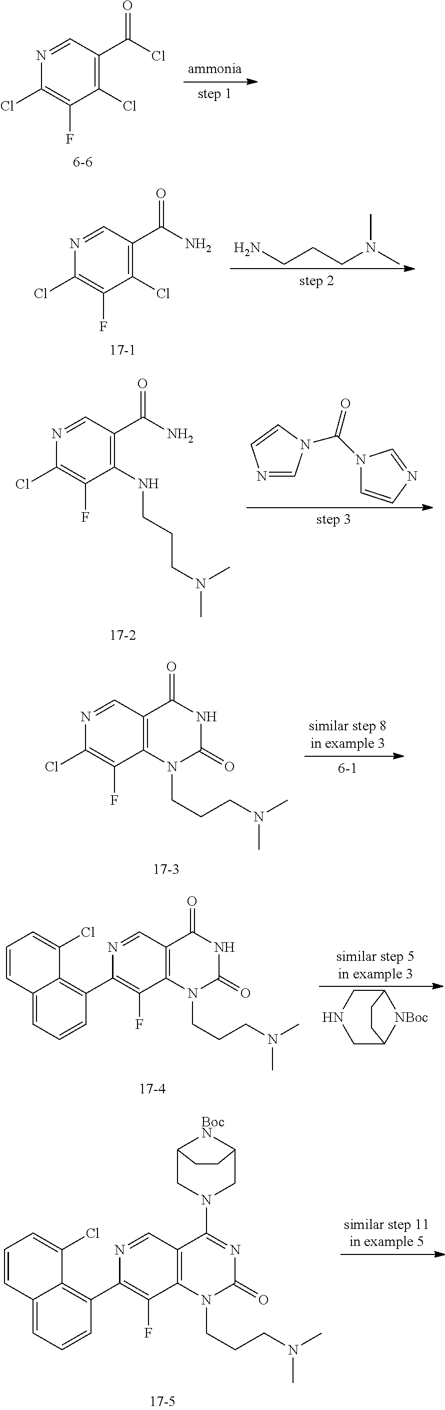

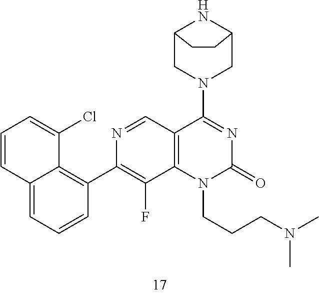 [SMILES: CN(C)CCCn1c(=O)nc(N2CC3CCC(C2)N3)c2cnc(-c3cccc4cccc(Cl)c34)c(F)c21]

Step 1: A solution of 6-6 (2.28 g, 10 mmol) in dioxane (5 mL) was added dropwise to ammonia (28%, 20 mL) at 0° C. Upon completion of the addition, the mixture was allowed to stir for additional 5 min and then filtered. The filter cake was collected and dried to afford 17-1 which was used directly in the next step without purification.

Step 2: A mixture of 17-1 (836 mg, 4.0 mmol) and N,N′-dimethylpropane-1,3-diamine (1.23 g, 12 mmol) in tetrahydrofuran (20 mL) was stirred at room temperature for 6 hours. Then the reaction mixture was diluted with water and extracted with tetrahydrofuran/ethyl acetate (1/1). The organic layer was washed with brine, dried over sodium sulfate, filtered and concentrated to afford 17-2 which was used directly in the next step without purification.

Step 3: A mixture of 17-2 (685 mg, 2.49 mmol) and 1,1′-carbonyldiimidazole (1.2 g, 7.48 mmol) in dimethylacetamide (4 mL) was stirred at 120° C. for 2 hours. Then the mixture was cooled and purified by reverse phase HPLC (acetonitrile with 0.05% of ammonium in water: 5% to 95%) to afford 17-3.

Compound 17-4 was prepared from 17-3 and 6-1 following the coupling procedure for the synthesis of compound 10 in example 3.

Compound 17-5 was prepared from 17-4 following the procedure for the synthesis of compound 10-5 in example 3.

Compound 17 was prepared from 17-5 following the procedure for the synthesis of compound 60 in example 5 as a 3 eq of TFA salt. LCMS (ESI, m/z): [M+H]⁺=521.2; ¹H NMR (400 MHz, methanol-d₄, ppm): δ 8.85 (s, 1H), 8.16-8.13 (m, 1H), 8.03-8.00 (m, 1H), 7.68 (t, J=7.6 Hz, 1H), 7.63-7.59 (m, 2H), 7.52 (d, J=7.6 Hz, 1H), 4.72-4.66 (m, 2H), 4.37-4.14 (m, 4H), 3.90-3.83 (m, 2H), 3.23-3.18 (m, 2H), 2.85 (s, 6H), 2.23-2.08 (m, 6H). ¹⁹F NMR (376 MHz, methanol-d₄, ppm): δ −136.34 (s, 1F).

### Example 12 Synthesis of Compound 35

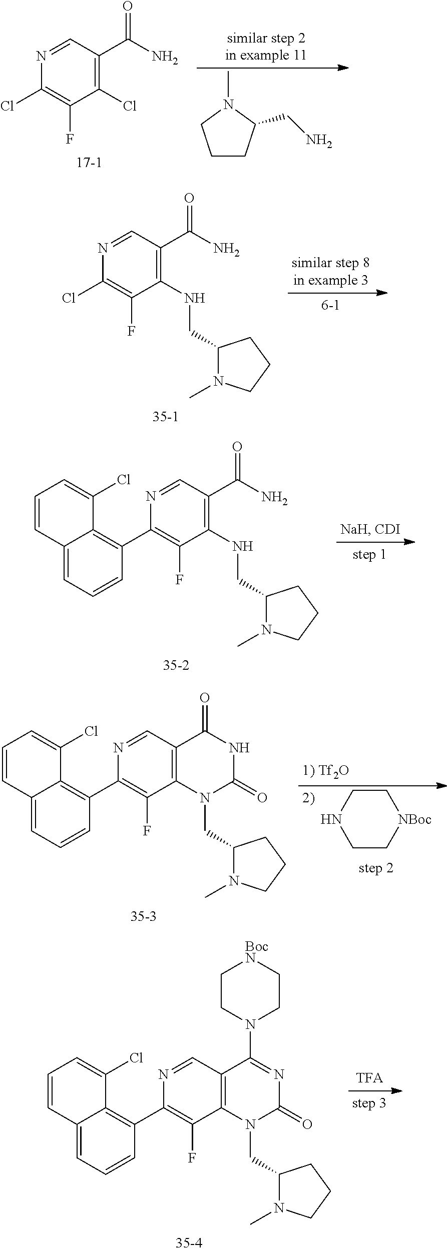

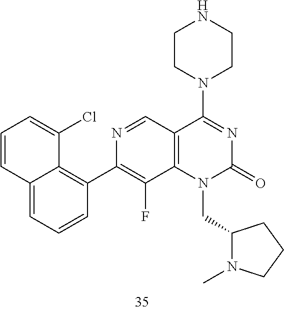 [SMILES: CN1CCC[C@H]1Cn1c(=O)nc(N2CCNCC2)c2cnc(-c3cccc4cccc(Cl)c34)c(F)c21]

Compound 35-1 was prepared from 17-1 following the procedure for the synthesis of compound 17-2 in example 11.

Compound 35-2 was prepared from 35-1 following the coupling procedure for the synthesis of compound 10 in example 3.

Step 1: To a solution of 35-2 (536 mg, 1.3 mmol) in N,N-dimethylformamide (15 mL) was added sodium hydride (208 mg, 5.2 mmol, 60% in mineral oil) at 0° C. in portions under N₂ atmosphere. The mixture was stirred at room temperature for 0.5 hour. To above mixture was added 1,1′-carbonyldiimidazole (421 mg, 2.6 mmol) and the resulting mixture was stirred for 2 hours. After being cooled to 0° C., the mixture was quenched with acetic acid, diluted with ethyl acetate, washed with sat. aqueous sodium bicarbonate and brine. The organic layer was dried over sodium sulfate, filtered and concentrated. The residue was purified by reverse phase HPLC (acetonitrile with 0.05% of trifluoroacetic acid in water: 5% to 95%) to afford 35-3.

Step 2: To a solution of 35-3 (110 mg, 0.25 mmol) and N,N-diisopropylethylamine (130 mg, 1 mmol) in dichloromethane (5 mL) was added trifluoromethanesulfonic anhydride (155 mg, 0.55 mmol) at 0° C. The mixture was stirred at room temperature for 1 hour under N₂ atmosphere. To above mixture was added N,N-diisopropylethylamine (65 mg, 0.5 mmol) followed by 1-Boc-piperazine (93 mg, 0.5 mmol) and stirred for 30 min. The mixture was diluted with dichloromethane and washed with brine. The mixture was dried over sodium sulfate, filtered and concentrated. The residue was purified by prep-HPLC (acetonitrile with 0.05% of TFA in water: 10% to 95%) to give afford 35-4.

Step 3: To a solution of 35-4 (32 mg, 0.05 mmol) in dichloromethane (1 mL) was added trifluoroacetic acid (0.25 mL). The mixture was stirred at 25° C. for 1 hour and then concentrated. The residue was purified by prep-HPLC (acetonitrile with 0.05% of TFA in water: 5% to 95%) to afford 35 as a 3 eq of TFA salt. LCMS (ESI, m/z): [M+H]⁺=507.3; ¹H NMR (400 MHz, methanol-d₄, ppm): δ 8.92 (s, 1H), 8.15 (dd, J=8.0, 0.8 Hz, 1H), 8.03-8.01 (m, 1H), 7.69 (t, J=7.2 Hz, 1H), 7.64-7.60 (m, 2H), 7.52 (t, J=8.0 Hz, 1H), 4.70-4.65 (m, 1H), 4.57-4.49 (m, 1H), 4.26-4.15 (m, 4H), 3.86-3.70 (m, 2H), 3.48-3.46 (m, 4H), 3.25-3.15 (m, 1H), 2.97-2.95 (m, 3H), 2.35-2.25 (m, 1H), 2.15-1.99 (m, 2H), 1.96-1.84 (m, 1H). ¹⁹F NMR (376 MHz, methanol-d₄, ppm): δ −134.97 (1F).

### Example 13 Synthesis of Compound 44

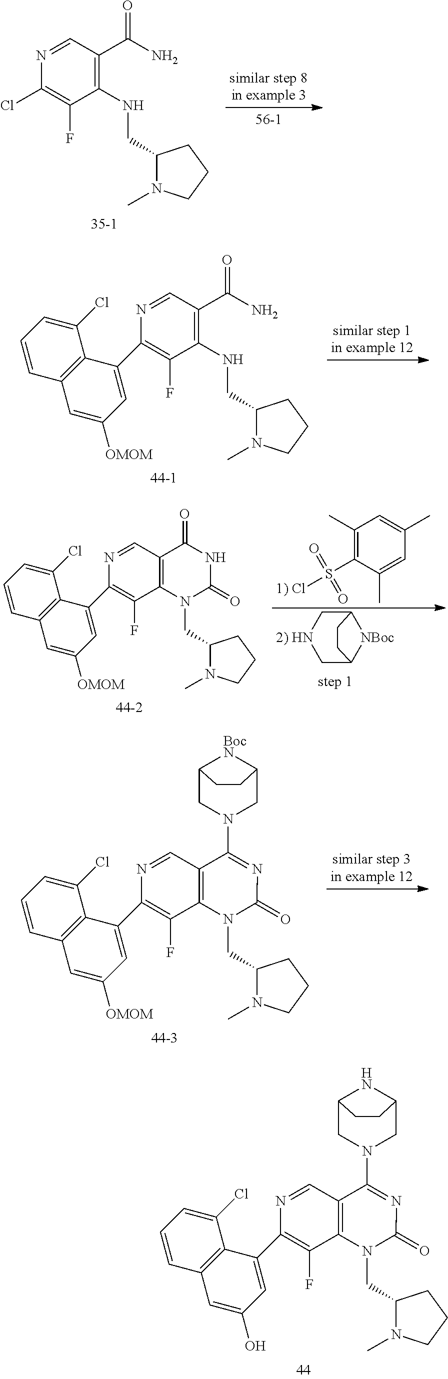

Compound 44-1 was prepared from 35-1 following the coupling procedure for the synthesis of compound 10 in example 3.

Compound 44-2 was prepared from 44-1 following the procedure for the synthesis of compound 35-3 in example 12.

Step 1: To a solution of 44-2 (115 mg, 0.23 mmol) and potassium carbonate (191 mg, 1.39 mmol) in acetonitrile (10 mL) was added 2,4,6-trimethylbenzenesulfonyl chloride (152 mg, 0.69 mmol) at 0° C. The mixture was stirred at room temperature for 16 hours. To above mixture was added a solution of tert-butyl (1R,5S)-3,8-diazabicyclo[3.2.1]octane-8-carboxylate (98 mg, 0.46 mmol) in acetonitrile (3 mL) and the resulting mixture was stirred at room temperature for 10 min. The mixture was diluted with ethyl acetate, washed with water and brine. The organic layer was dried over sodium sulfate, filtered and concentrated. The residue was purified by reverse phase HPLC (acetonitrile with 0.05% of trifluoroacetic acid in water: 5% to 95%) to afford 44-3.

Compound 44 was prepared from 44-3 following the procedure for the synthesis of compound 35 in example 12 as a 2 eq of TFA salt. LCMS (ESI, m/z): [M+H]⁺=549.3; ¹H NMR (400 MHz, methanol-d₄, ppm): δ 8.87 (s, 1H), 7.76 (dd, J=7.2, 2.0 Hz, 1H), 7.40-7.35 (m, 3H), 7.17 (dd, J=6.8, 2.4 Hz, 1H), 4.85-4.80 (m, 1H), 4.72-4.62 (m, 2H), 4.58-4.44 (m, 1H), 4.28-4.17 (m, 2H), 3.99-3.95 (m, 1H), 3.88-3.68 (m, 3H), 3.30-3.16 (m, 1H), 2.96-2.95 (m, 3H), 2.37-2.24 (m, 1H), 2.20-1.97 (m, 6H), 1.96-1.83 (m, 1H). ¹⁹F NMR (376 MHz, methanol-d₄, ppm): δ −134.89 (1F).

### Example 14 Synthesis of Compound 18

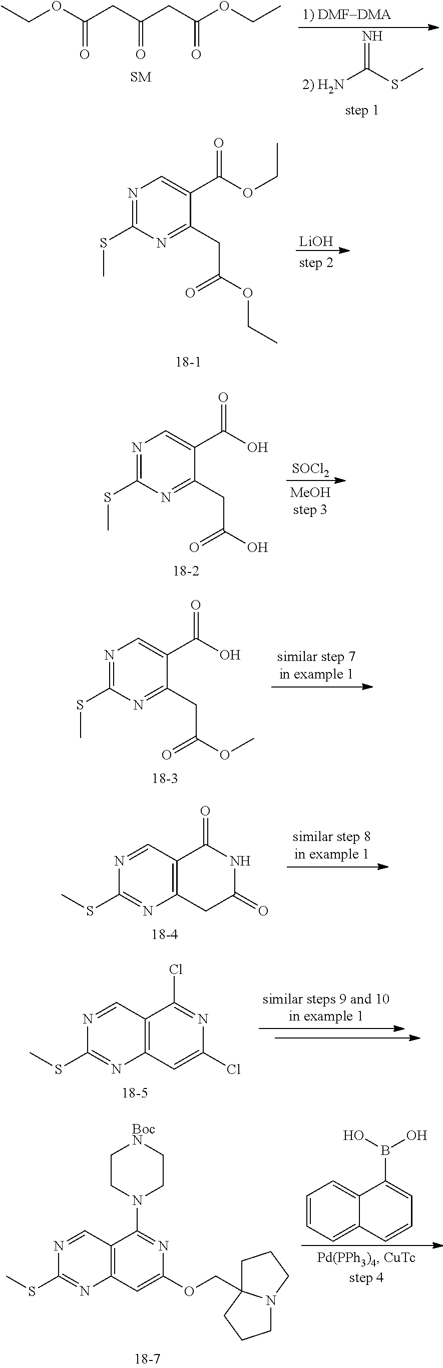

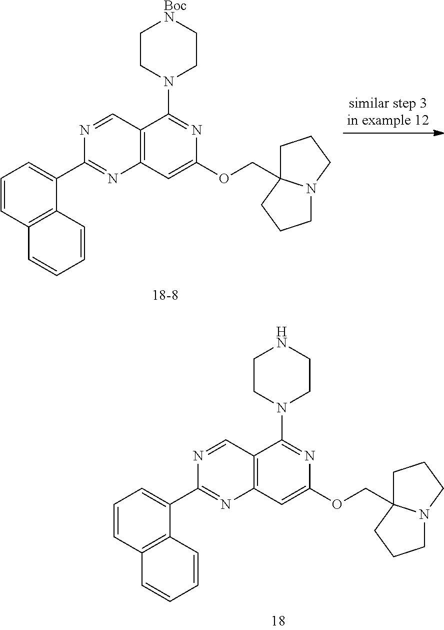 [SMILES: CC(C)(C)OC(=O)N1CCN(c2nc(OCC34CCCN3CCC4)cc3nc(-c4cccc5ccccc45)ncc23)CC1 ; c1ccc2c(-c3ncc4c(N5CCNCC5)nc(OCC56CCCN5CCC6)cc4n3)cccc2c1]

Step 1: To a solution of diethyl 3-oxopentanedioate (40.3 mL, 222 mmol) in ethanol (400 mL) was added 1,1-dimethoxy-N,N-dimethylmethanamine (29.5 mL, 222 mmol) and the mixture was stirred at room temperature for 45 min. Methyl carbamimidothioate sulfate (30 g, 222 mmol) was then added and the mixture was stirred at reflux for 8 hours. The mixture was diluted with ethyl acetate and washed with water. The organic layer was dried over sodium sulfate, filtered and concentrated to afford 18-1 which was used directly in the next step without purification.

Step 2: 1 M aqueous LiOH solution (200 mL) was added to a solution of 18-1 (15 g, 53 mmol) in tetrahydrofuran (200 mL) at 0° C. The resulting mixture was stirred at room temperature for 16 hours. Tetrahydrofuran was removed under reduced pressure and the pH of the residue was adjusted to around 3 with 2 M HCl at 0° C. The mixture was filtered and dried to afford 18-2 which was used directly in the next step without purification.

Step 3: To a solution of 18-2 (5 g, 22 mmol) in methanol (100 mL) was added SOCl₂ (5.2 g, 44 mmol) at 0° C. The reaction was stirred at room temperature for 2 hours. The mixture was concentrated. The residue was diluted with ethyl acetate and washed with water. The organic layer was dried over anhydrous sodium sulfate, filtered and concentrated to afford 18-3 which was used directly in the next step without purification.

Compound 18-4 was prepared from 18-3 following the procedure for the synthesis of compound 20-7 in example 1.

Compound 18-5 was prepared from 18-4 following the procedure for the synthesis of compound 20-8 in example 1.

Compound 18-7 was prepared from 18-5 following the procedure for the synthesis of compound 20-10 in example 1.

Step 4: To a solution of 18-7 (25 mg, 0.05 mmol) in tetrahydrofuran (6 mL) was added naphthalen-1-ylboronic acid (25 mg, 0.15 mmol), copper(I) thiophene-2-carboxylate (28 mg, 0.15 mmol) and tetrakis(triphenylphosphine) palladium (0) (25 mg, 0.022 mmol). The mixture was stirred at 85° C. for 1 hour under nitrogen under microwave condition. The mixture was cooled, diluted with ethyl acetate and washed with water. The organic layer was dried over anhydrous sodium sulfate, filtered and concentrated. The residue was purified by column chromatography on silica gel (dichloromethane to dichloromethane/methanol/ammonia=100/10/0.5) to afford 18-8.

Compound 18 was prepared from 18-8 following the procedure for the synthesis of compound 35 in example 12 as a 3 eq of TFA salt. LCMS (ESI, m/z): [M+H]⁺=481.3; ¹H NMR (400 MHz, methanol-d₄, ppm): δ 9.60 (d, J=0.8 Hz, 1H), 8.53 (d, J=7.2 Hz, 1H), 8.07 (d, J=7.6 Hz, 2H), 7.99-7.97 (m, 1H), 7.63 (t, J=8.0 Hz, 1H), 7.56-7.52 (m, 2H), 6.84 (d, J=0.8 Hz, 1H), 4.63 (s, 2H), 4.03-4.00 (m, 4H), 3.70-3.63 (m, 2H), 3.53-3.51 (m, 4H), 3.35-3.31 (m, 2H), 2.34-2.10 (m, 8H).

### Example 15 Synthesis of Compound 75

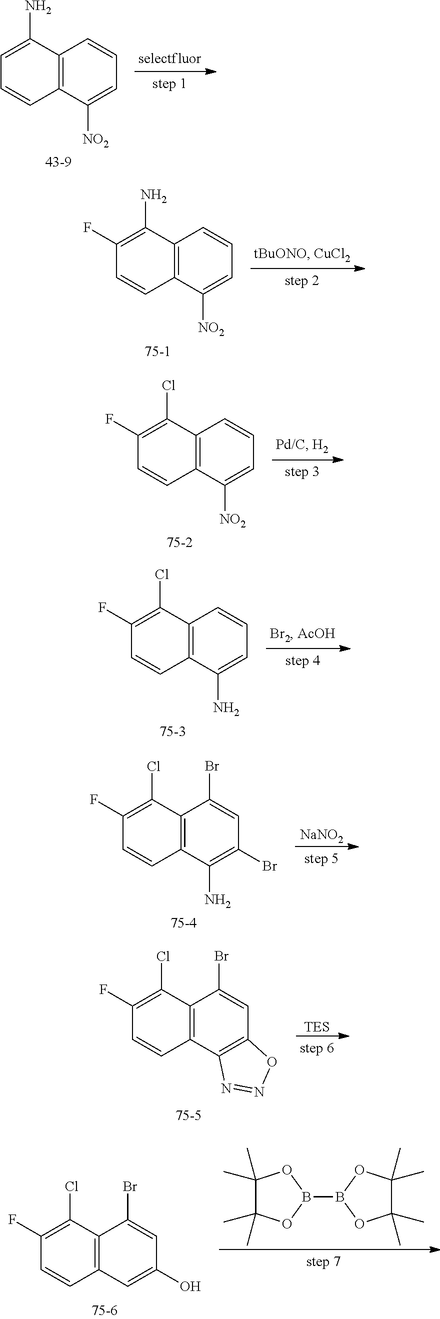

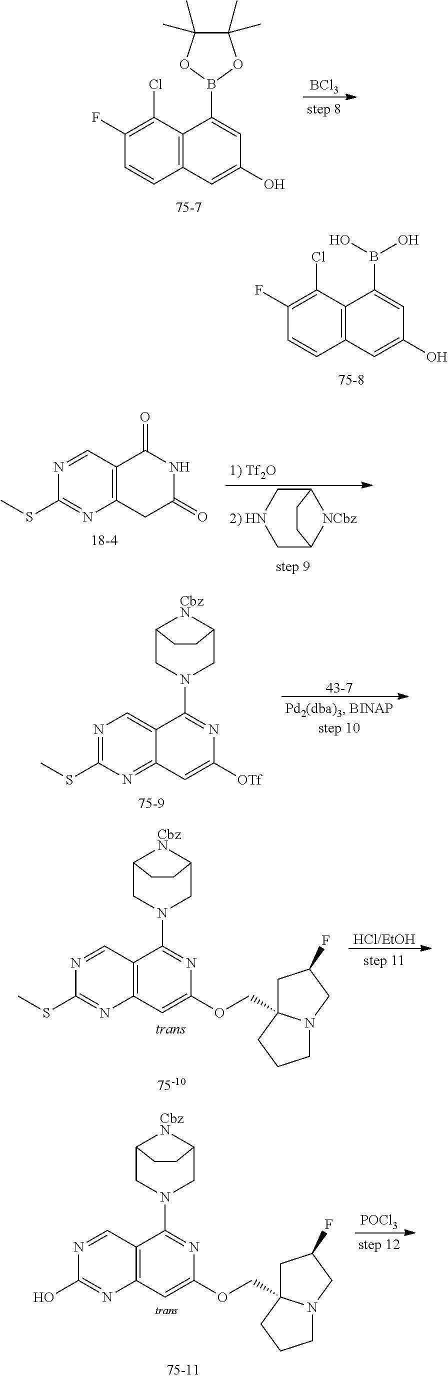

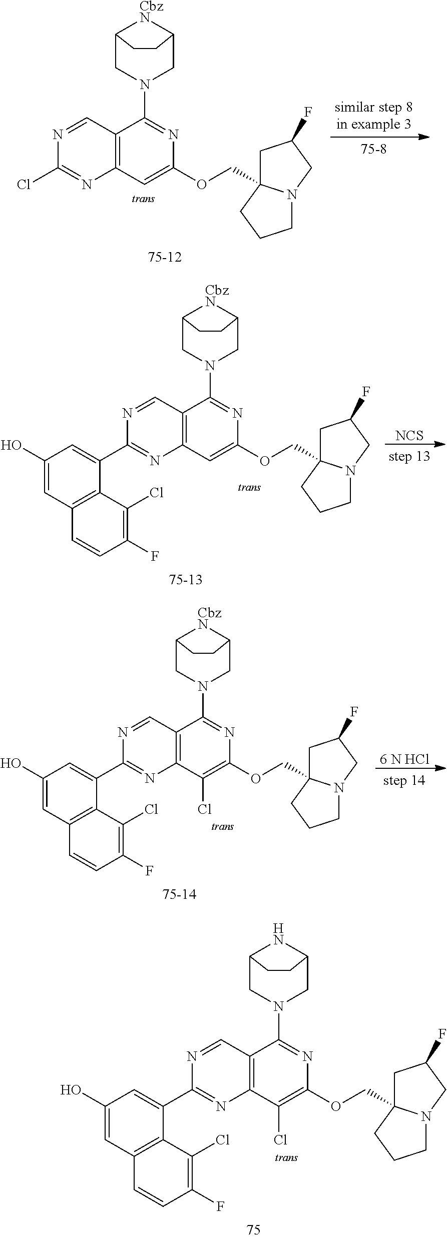

Step 1: A mixture of 43-9 (19 g, 101 mmol), triethylamine (20.4 g, 202 mmol), selectfluor (93 g, 263 mmol) in ethanol/1-Methyl-2-pyrrolidinone (150 mL/150 mL) was stirred at room temperature overnight under N₂ atmosphere. The mixture was diluted with water and extracted with ethyl acetate. The combined organic layers were washed with water and brine, dried over anhydrous Na₂SO₄, filtered and concentrated to afford 75-1.

Step 2: To a mixture of 75-1 (21 g, 105 mmol) and copper chloride (15.5 g, 115.5 mmol) in acetonitrile (200 mL) was added tert-butyl nitrite (16.2 g, 57.5 mmol) under N₂ atmosphere at 0° C. Then the mixture was stirred at room temperature for 2 hours before it was diluted with water and extracted with ethyl acetate. The combined organic layers were washed with water and brine, dried over anhydrous Na₂SO₄, filtered and concentrated. The residue was purified by column chromatography on silica gel (petroleum ether to petroleum ether/ethyl acetate=10/1) to afford 75-2.

Step 3: A mixture of 75-2 (18.6 g, 83 mmol) and 5% Pd/C (2.0 g) in ethyl acetate (200 mL) was stirred at room temperature for 24 hours under hydrogen atmosphere. The mixture was filtered and concentrated to give a residue which was purified by column chromatography on silica gel (petroleum ether to petroleum ether/ethyl acetate=4/1) and prep-HPLC (acetonitrile with 0.05% of TFA in water: 25% to 95%) to afford 75-3.

Step 4: To a mixture of 75-3 (6.6 g, 33.8 mmol) in acetic acid (300 mL) was added bromine (11.9 g, 74.5 mmol) at room temperature. The mixture was stirred at 70° C. for 6 hours. Then the suspension was filtered and the filtrate was concentrated to afford 75-4.

Step 5: To a solution of 75-4 (9.1 g, 25.9 mmol) in acetic acid/propionic acid (100 mL/25 mL) was added sodium nitrite (2.15 g, 31 mmol) at 0° C. The mixture was stirred at 0° C. for 1 h. The mixture was diluted with water and extracted with dichloromethane. The combined organic layers were dried over anhydrous Na₂SO₄, filtered and concentrated to afford 75-5.

Step 6: To a mixture of 75-5 (8.3 g, 27.7 mmol) in isopropyl alcohol (200 mL) was added triethylsilane (6.42 g, 55.3 mmol). The mixture was stirred at 100° C. overnight under N₂ atmosphere. Then concentrated and the residue was purified by column chromatography on silica gel (petroleum ether to petroleum ether/ethyl acetate=4/1) to afford 75-6.

Step 7: To a mixture of 75-6 (2.0 g, 7.3 mmol) in dioxane (30 mL) was added 4,4,4′,4′,5,5,5′,5′-octamethyl-2,2′-bi(1,3,2-dioxaborolane) (2.4 g, 9.5 mmol), potassium acetate (2.15 g, 21.9 mmol) and [1,1′-bis(diphenylphosphino)ferrocene]dichloropalladium(II) (534 mg, 0.73 mmol). The mixture was stirred at 95° C. for 4 hours under N₂ atmosphere. Then the suspension was filtered and the filtrate was diluted with water and extracted with ethyl acetate. The combined organic layers were dried over anhydrous Na₂SO₄, filtered and concentrated. The residue was purified by column chromatography on silica gel (petroleum ether to petroleum ether/ethyl acetate=4/1) to afford 75-7.

Step 8: To a solution of 75-7 (1 g, 3.1 mmol) in dichloromethane (5 mL) was added boron chloride (1.0 M in methylene chloride, 6.2 mL, 6.2 mmol) at room temperature. The mixture was stirred at room temperature for 2 hours. The mixture was diluted with ice water and extracted with dichloromethane. The combined organic layers were dried over anhydrous Na₂SO₄, filtered and concentrated. The residue was purified by prep-HPLC (acetonitrile with 0.05% of TFA in water: 5% to 95%) to afford 75-8.

Step 9: To the solution of 18-4 (10.0 g, 47.8 mmol) and N, N-diisopropylethylamine (24.6 g, 191.2 mmol) in dichloromethane (250 mL) was added trifluoromethanesulfonic anhydride (33.4 g, 119.0 mmol) at −20° C. The mixture was stirred at −20° C. for 50 min. To the above mixture was added a solution of benzyl 3,8-diazabicyclo[3.2.1]octane-8-carboxylate (15.0 g, 71.7 mmol) in dichloromethane (50 mL) at −20° C. The reaction was stirred at 0° C. for 10 min. The mixture was quenched with water and extracted with dichloromethane. The combined organic layers were dried over sodium sulfate, filtered and concentrated. The residue was purified by silica-gel column chromatography (petroleum ether to petroleum ether/ethyl acetate=10/3) to afford 75-9.

Step 10: To a solution of 75-9 (1.66 g, 2.9 mmol), 43-7 (0.928 g, 5.8 mmol) and cesium carbonate (2.84 g, 8.7 mmol) in toluene (30 mL) was added tris(dibenzylideneacetone)dipalladium (266 mg, 0.29 mmol) and 2,2′-bis(diphenylphosphino)-1,1′-binaphthalene (361 mg, 0.58 mmol). The mixture was stirred at 110° C. for 3 hours under N₂ atmosphere. The reaction was cooled, poured into water and extracted with ethyl acetate. The combined organic layers were dried over sodium sulfate, filtered and concentrated. The residue was purified by silica gel column chromatography (dichloromethane/methanol/ammonium hydroxide=10/1/0.05) to afford 75-10.

Step 11: To a solution of 75-10 (0.97 g, 1.7 mmol) in ethanol (15 mL) was added 3 M HCl (15 mL). The reaction was stirred at 50° C. for 6 hours. The mixture was cooled, poured into saturated aqueous sodium bicarbonate solution and extracted with dichloromethane. The combined organic layers were dried over sodium sulfate, filtered and concentrated to afford 75-11 which was used directly in the next step without purification.

Step 12: A solution of 75-11 (340 mg, 0.62 mmol) in phosphorus oxychloride (8 mL) was stirred at 105° C. for 30 min and then concentrated. The residue was diluted with dichloromethane and washed with saturated aqueous sodium bicarbonate. The organic layer was concentrated and purified by reverse phase HPLC (acetonitrile with 0.05% of TFA in water: 5% to 50%) to afford 75-12.

Compound 75-13 was prepared from 75-12 and 75-8 following the coupling procedure for the synthesis of compound 10 in example 3.

Step 13: To a solution of 75-13 (16 mg, 0.02 mmol) in dimethylformamide (10 mL) was added N-chlorosuccinimide (3.3 mg, 0.025 mmol). The reaction was stirred for 16 hours at room temperature. The mixture was extracted with ethyl acetate and washed with water. The organic layer was concentrated and purified by reverse phase HPLC (acetonitrile with 0.05% of TFA in water: 15% to 75%) to afford 75-14.

Step 14: To a solution of 75-14 (9 mg, 0.012 mmol) in ethanol (3 mL) was added 6 N hydrochloric acid (3 mL). The reaction was stirred at 90° C. for 4 hours. The mixture was poured into saturated aqueous sodium bicarbonate and extracted with dichloromethane. The organic layer was concentrated. The residue was purified by prep-HPLC (acetonitrile with 0.05% of TFA in water: 10% to 40%) to afford 75 as 3 eq of TFA salt. LCMS (ESI, m/z): [M+H]⁺=627.2; ¹H NMR (400 MHz, methanol-d₄, ppm): δ 9.43 (s, 1H), 7.82-7.78 (m, 1H), 7.41-7.33 (m, 3H), 5.69-5.57 (m, 1H), 4.40-3.82 (m, 4H), 4.25-4.22 (m, 2H), 3.95-3.82 (m, 5H), 3.52-3.50 (m, 1H), 2.79-2.17 (m, 10H).

### Example 16 Synthesis of Compound 61

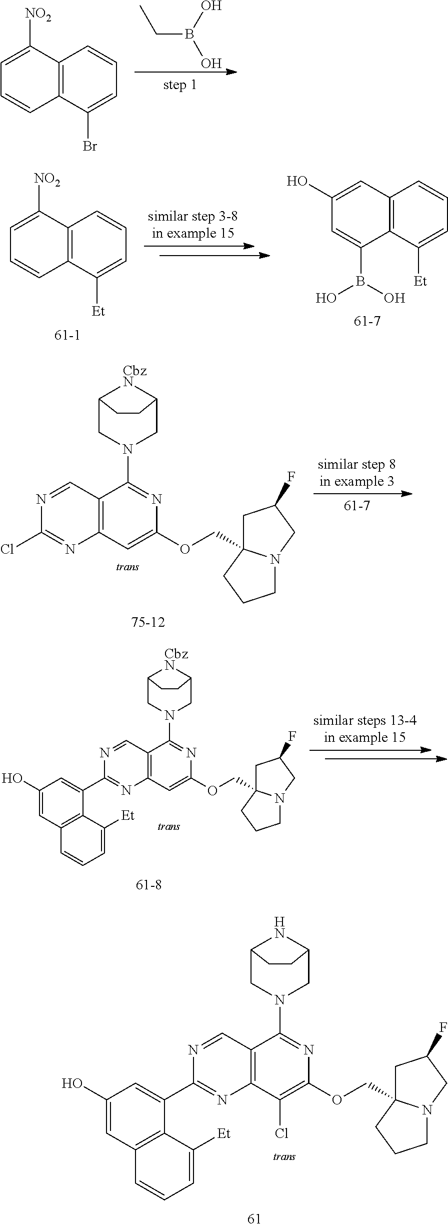

Step 1: To a mixture of potassium phosphate (176 g, 714 mmol) in toluene/water (896 mL/112 mL) was added 5-bromo-1-nitro-naphthalene (70 g, 278 mmol), ethylboronic acid (41.15 g, 556 mmol), and [1,1′-bis(diphenylphosphino)ferrocene]dichloropalladium(II) (10.1 g, 13.9 mmol) under nitrogen atmosphere. The mixture was stirred at 100° C. for 16 hours. The mixture was filtered and the filtrate was washed with water and brine. The organic layer was dried over sodium sulfate, filtered and concentrated. The residue was purified by column chromatography on silica gel (petroleum ether to petroleum ether/ethyl acetate=95/5) to afford 61-7.

Compound 61-7 was prepared from 61-1 following the procedure for the synthesis of compound 75-8 in example 15.

Compound 61-8 was prepared from 75-12 following the coupling procedure for the synthesis of compound 10 in example 3.

Compound 61 was prepared from 61-8 following the procedure for the synthesis of compound 75 in example 15 as a 3 eq of TFA salt. LCMS (ESI, m/z): [M+H]⁺=603.3; ¹H NMR (400 MHz, methanol-d₄, ppm): δ 9.46 (s, 1H), 7.62 (d, J=7.6 Hz, 1H), 7.37 (t, J=8.0 Hz, 1H), 7.28 (d, J=2.4 Hz, 1H), 7.19 (d, J=7.2 Hz, 1H), 7.12 (d, J=2.4 Hz, 1H), 5.71-5.70 (m, 1H), 4.76-4.66 (m, 2H), 4.53-4.50 (m, 2H), 4.26-4.22 (m, 2H), 3.97-3.84 (m, 5H), 3.52-3.50 (m, 1H), 2.78-2.16 (m, 12H), 0.93 (t, J=7.6 Hz, 3H).

### Example 17 Synthesis of Compound 59

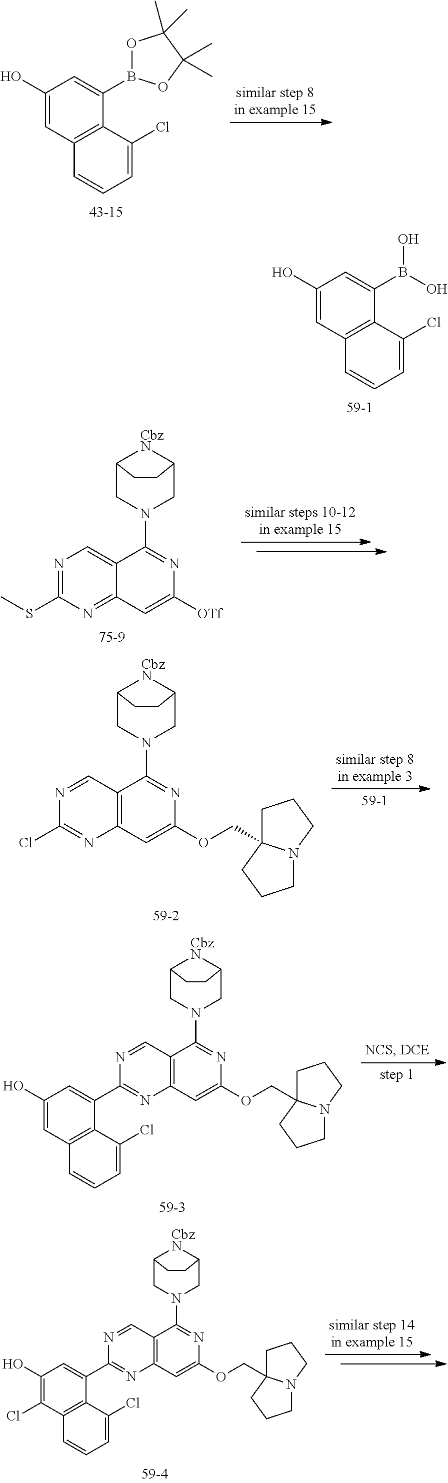

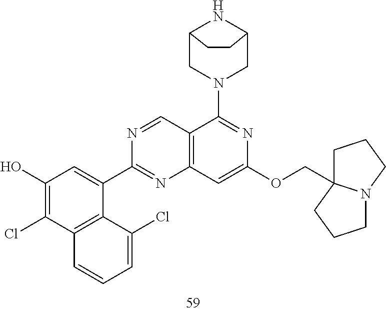 [SMILES: Oc1cc(-c2ncc3c(N4CC5CCC(C4)N5)nc(OCC45CCCN4CCC5)cc3n2)c2c(Cl)cccc2c1Cl]

Compound 59-1 was prepared from 43-15 following the procedure for the synthesis of compound 75-8 in example 15.

Compound 59-2 was prepared from 75-9 following the procedure for the synthesis of compound 75-12 in example 15.

Compound 59-3 was prepared from 59-1 and 59-2 following the coupling procedure for the synthesis of compound 10 in example 3.

Step 1: To a solution of 59-3 (30 mg, 0.04 mmol) in dichloroethane (10 mL) was added N-chlorosuccinimide (5 mg, 0.04 mmol). The reaction was stirred for 2 hours at room temperature. The mixture was extracted with dichloromethane and washed with water. The organic phase was concentrated and the residue was purified by prep-HPLC (acetonitrile with 0.05% of TFA in water: 10% to 50%) to afford 59-4.

Compound 59 was prepared from 59-4 following the procedure for the synthesis of compound 75 in example 15 as a 3 eq of TFA salt. LCMS (ESI, m/z): [M+H]⁺=591.3; ¹H NMR (400 MHz, methanol-d₄, ppm): δ 9.43 (s, 1H), 8.28 (dd, J=8.4, 1.2 Hz, 1H), 7.57-7.53 (m, 1H), 7.46-7.44 (m, 1H), 7.38 (s, 1H), 6.68 (s, 1H), 4.61 (d, J=1.6 Hz, 2H), 4.55 (d, J=14 Hz, 1H), 4.42 (d, J=13.2 Hz, 1H), 4.23 (s, 2H), 3.89-3.80 (m, 2H), 3.68-3.62 (m, 2H), 3.37-3.34 (m, 2H), 2.34-2.09 (m, 12H).

### Example 18 Synthesis of Compound 38

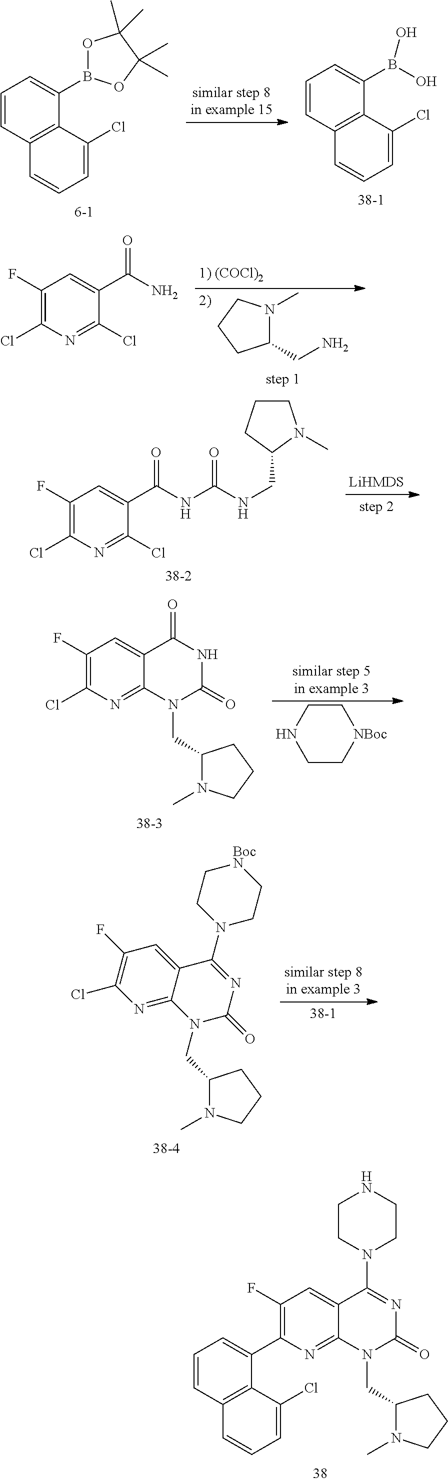

Compound 38-1 was prepared from 6-1 following the procedure for the synthesis of compound 75-8 in example 15.

Step 1: To a solution of 2,6-dichloro-5-fluoronicotinamide (6.24 g, 30.0 mmol) in DCE (40 mL) was added dropwise oxalyl chloride (7.62 g, 60.0 mmol) at room temperature. The mixture was stirred at 80° C. for 1 hour under nitrogen atmosphere. The mixture was concentrated and the residue was re-dissolved in THF (40 mL) and to this solution was added dropwise (S)-(1-methylpyrrolidin-2-yl)methanamine (3.42 g, 30.0 mmol) at −35° C. The resulting mixture was stirred for 1 hour at room temperature before it was quenched with water and extracted with ethyl acetate. The combined organic layers were dried over anhydrous sodium sulfate, filtered and concentrated. The residue was triturated with petroleum ether/ethyl acetate (20/1) to afford 38-2.

Step 2: To a solution of 38-2 (2.1 g, 6.0 mmol) in tetrahydrofuran (20 mL) was added dropwise 1 M LiHMDS solution in THF (12.4 mL, 12.4 mmol) at 0° C. The reaction solution was stirred for 2 hours at room temperature. After being quenched with saturated aqueous NH₄Cl solution, the mixture was extracted with ethyl acetate. The combined organic layers were dried over anhydrous sodium sulfate, filtered and concentrated. The residue was purified by column chromatography on silica gel (dichloromethane to dichloromethane/methanol=10/1) to afford 38-3.

Compound 38-4 was prepared from 38-3 following the procedure for the synthesis of compound 10-5 in example 3.

Compound 38 was prepared from 38-1 and 38-4 following the procedure for the synthesis of compound 10 in example 3 as a 2 eq of TFA salt. LCMS (ESI, m/z): [M+H]⁺=507.3; ¹H NMR (400 MHz, methanol-d₄, ppm): δ 8.26-8.23 (m, 1H), 8.17-8.15 (m, 1H), 8.04-8.02 (m, 1H), 7.71-7.62 (m, 3H), 7.56-7.52 (m, 1H), 4.79-4.61 (m, 2H), 4.20-4.10 (m, 4H), 3.80-3.62 (m, 2H), 3.52-3.41 (m, 4H), 3.15-3.05 (m, 1H), 2.91 (d, J=17.6 Hz, 3H), 2.25-1.85 (m, 4H).

### Example 19 Synthesis of Compound 45

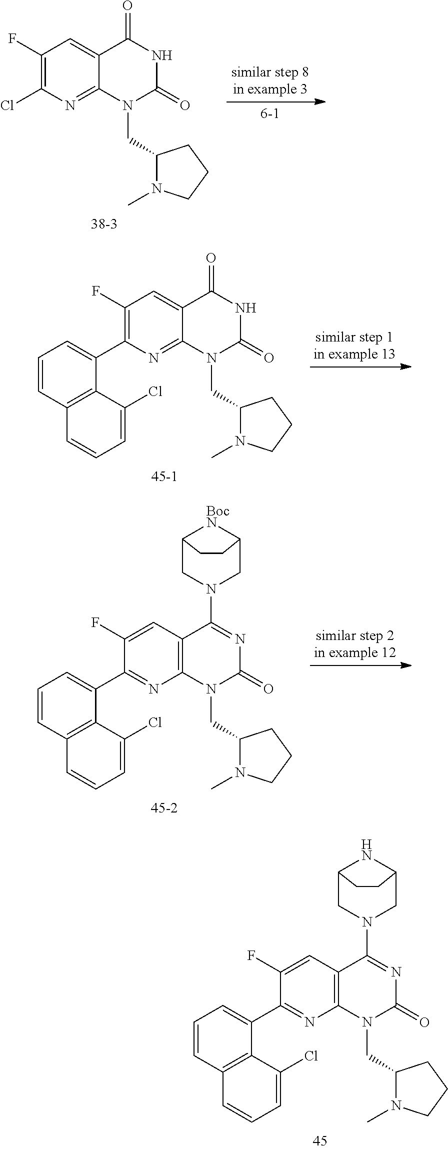 [SMILES: CN1CCC[C@H]1Cn1c(=O)[nH]c(=O)c2cc(F)c(Cl)nc21 ; CN1CCC[C@H]1Cn1c(=O)[nH]c(=O)c2cc(F)c(-c3cccc4cccc(Cl)c34)nc21 ; CN1CCC[C@H]1Cn1c(=O)nc(N2CC3CCC(C2)N3C(=O)OC(C)(C)C)c2cc(F)c(-c3cccc4cccc(Cl)c34)nc21 ; CN1CCC[C@H]1Cn1c(=O)nc(N2CC3CCC(C2)N3)c2cc(F)c(-c3cccc4cccc(Cl)c34)nc21]

Compound 45-1 was prepared from 6-1 and 38-3 following the coupling procedure for the synthesis of compound 10 in example 3.

Compound 45-2 was prepared from 45-1 following the procedure for the synthesis of compound 44-3 in example 13.

Compound 45 was prepared from 45-2 following the procedure for the synthesis of compound 35 in example 12 as a 2 eq of TFA salt. LCMS (ESI, m/z): [M+H]⁺=533.3; ¹H NMR (400 MHz, methanol-d₄, ppm): δ 8.20-8.15 (m, 2H), 8.04-8.02 (m, 1H), 7.71-7.62 (m, 3H), 7.56-7.52 (m, 1H), 4.91-4.80 (m, 1H), 4.80-4.60 (m, 3H), 4.30-4.20 (m, 2H), 3.90-3.62 (m, 4H), 3.15-3.05 (m, 1H), 2.91 (d, J=17.6 Hz, 3H), 2.25-1.85 (m, 8H).

### Example 20 Synthesis of Compound 50

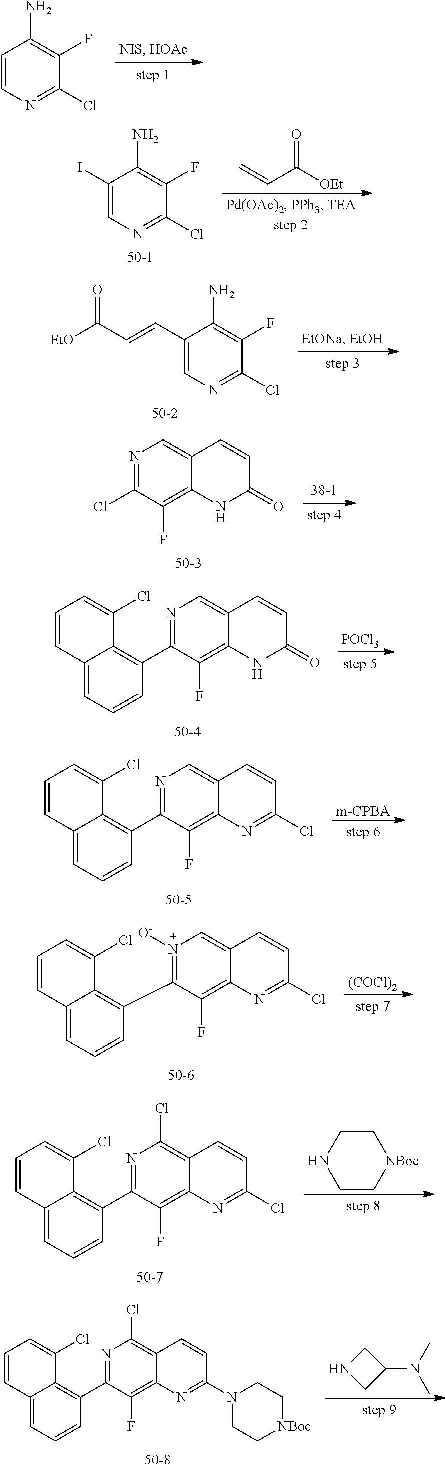

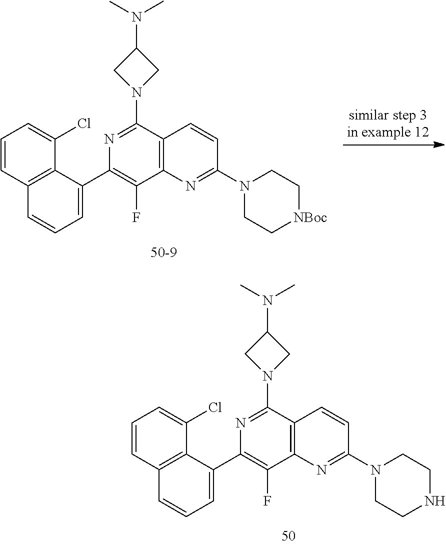 [SMILES: CN(C)C1CN(c2nc(-c3cccc4cccc(Cl)c34)c(F)c3nc(N4CCN(C(=O)OC(C)(C)C)CC4)ccc23)C1 ; CN(C)C1CN(c2nc(-c3cccc4cccc(Cl)c34)c(F)c3nc(N4CCNCC4)ccc23)C1]

Step 1: To a solution of 2-chloro-3-fluoropyridin-4-amine (5 g, 34.12 mmol) in acetic acid (65 mL) was added NIS (11.5 g, 51.18 mmol). The resulting mixture was stirred at 120° C. for 2.5 hours. The resulting mixture was cooled to room temperature and concentrated under vacuum. The residue was diluted with ethyl acetate, washed with saturated aqueous sodium bicarbonate and brine. The combined organic layers were dried over anhydrous sodium sulfate, filtered and concentrated to afford 50-1.

Step 2: A mixture of 50-1 (1.0 g, 3.68 mmol), ethyl acrylate (464 mg, 5.51 mmol), Pd(OAc)(66 mg, 0.29 mmol), PPh₃ (116 mg, 0.44 mmol) and TEA (740 mg, 7.33 mmol) in 1,4-dioxane (30 mL) was stirred at 90° C. for 3 hours under nitrogen atmosphere. The resulting mixture was cooled, diluted with water and extracted with ethyl acetate. The combined organic layers were dried over sodium sulfate, filtered and concentrated. The residue was purified by column on silica gel (petroleum ether/ethyl acetate=10/7) to afford 50-2.

Step 3: To a stirred solution of 50-2 (400 mg, 1.63 mmol) in ethanol (7 mL) was added sodium ethoxide (154 mg, 2.26 mmol). The resulting mixture was stirred at 80° C. for 2 hours under N₂ atmosphere. The mixture was cooled, diluted with water and extracted with ethyl acetate. The combined organic layers were dried over sodium sulfate, filtered and concentrated to afford 50-3 which was used directly in the next step without purification.

Step 4: A mixture of 50-3 (1.2 g, 6.04 mmol), 38-1 (2.12 g, 10.3 mmol), Pd(PPh₃)₄ (700 mg, 0.607 mmol) and sodium carbonate (1.93 g, 18.2 mmol) in 1,4-dioxane (40 mL) and water (5 mL) was stirred at 100° C. for 5 hours under nitrogen atmosphere. The resulting mixture was cooled, diluted with water and extracted with ethyl acetate. The combined organic layers were dried over sodium sulfate, filtered. The residue was purified by silica gel column chromatography (petroleum ether/ethyl acetate=10/7) to afford 50-4.

Step 5: A solution of 50-4 (800 mg, 2.47 mmol) in phosphorus oxychloride (15 mL) was stirred at 90° C. for 2 hours. The resulting mixture was cooled and concentrated to afford 50-5 which was used directly in the next step without purification.

Step 6: To a solution of 50-5 (430 mg, 1.26 mmol) in dichloromethane (8 mL) was added m-CPBA (324 mg, 1.88 mmol). The mixture was stirred at 40° C. for 16 hours. The mixture was cooled, diluted with dichloromethane and washed with saturated aqueous sodium thiosulfate solution, saturated aqueous sodium bicarbonate solution and brine. The organic layer was dried over sodium sulfate, filtered and concentrated. The residue was purified by column chromatography on silica gel (ethyl acetate) to afford 50-6.

Step 7: To a solution of 50-6 (240 mg, 0.67 mmol) in dichloromethane (5 mL) was added oxalyl chloride (1.26 g, 3.35 mmol). The resulting mixture was stirred at 40° C. for 2 hours. The mixture was cooled, diluted with dichloromethane and washed with water. The organic layer was dried over sodium sulfate, filtered and concentrated to afford 50-7 which was used directly in the next step without purification.

Step 8: To a solution of 50-7 (280 mg, 0.74 mmol) in DMF (5 mL) was added DIEA (384 mg, 2.98 mmol) and tert-butyl piperazine-1-carboxylate (275 mg, 1.48 mmol). The resulting mixture was stirred at room temperature for 4 hours. The mixture was diluted with water and extracted with ethyl acetate. The combined organic layers were dried over sodium sulfate, filtered and concentrated. The residue was purified by silica gel column chromatography (ethyl acetate) to afford 50-8.

Step 9: To a solution of N,N-dimethylazetidin-3-amine (100 mg, 1.0 mmol) in 8 mL of DMSO was added 50-8 (120 mg, 0.23 mmol) and potassium carbonate (157 mg, 1.14 mmol). The resulting mixture was stirred at 120° C. for 8 hours. After being cooled to room temperature, the mixture was diluted with water and extracted with ethyl acetate. The combined organic layers were washed with brine, dried over anhydrous sodium sulfate, filtered and concentrated to afford 50-9 which was used directly in the next step without purification.

Compound 50 was prepared from 50-9 following the procedure for the synthesis of compound 35 in example 12 as a 3 eq of TFA salt. LCMS (ESI, m/z): [M+H]⁺=491.3; ¹H NMR (400 MHz, DMSO-d₆, ppm): δ 10.75 (brs, 1H), 9.15-9.05 (m, 2H), 8.16-8.11 (m, 2H), 8.04 (d, J=8.0 Hz, 1H), 7.69-7.64 (m, 1H), 7.60-7.55 (m, 2H), 7.53-7.48 (m, 1H), 7.23 (d, J=9.6 Hz, 1H), 4.54-4.49 (m, 1H), 4.44-4.40 (m, 1H), 4.36-4.27 (m, 2H), 4.25-4.15 (m, 1H), 4.05-3.90 (m, 4H), 3.30-3.15 (m, 4H), 2.78 (s, 6H). ¹⁹F NMR (376 MHz, DMSO-d₆, ppm): δ −152.25 (1F).

### Example 21 Synthesis of Compound 53

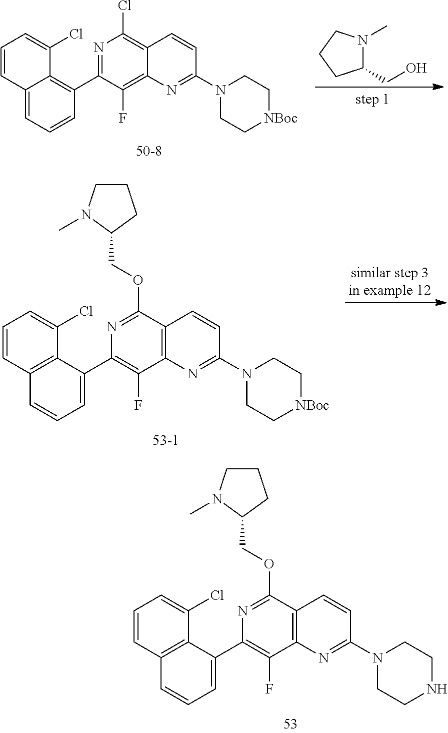

Step 1: To a solution of (R)-(1-methylpyrrolidin-2-yl) methanol (167 mg, 1.45 mmol) in PGP-2 DMF (4 mL) was added NaH (60% in mineral oil, 57 mg, 1.42 mmol) at 0° C. The resulting mixture was stirred at this temperature for 1 hour and then added dropwise to a solution of 50-8 (90 mg, 0.17 mmol) in DMF (2 mL). The reaction mixture was stirred at 60° C. for 2 hours. After being cooled to room temperature, the mixture was diluted with water and extracted with ethyl acetate. The combined organic layers were washed with brine, dried over anhydrous sodium sulfate, filtered and concentrated. The residue was purified by prep-TLC (petroleum ether/ethyl acetate=1/1) to afford 53-1.

Compound 53 was prepared from 53-1 following the procedure for the synthesis of compound 35 in example 12 as a 3 eq of TFA salt. LCMS (ESI, m/z): [M+H]⁺=506.3; ¹H NMR (400 MHz, DMSO-d₆, ppm): δ 9.88 (brs, 1H), 8.98 (brs, 2H), 8.36 (d, J=9.6 Hz, 1H), 8.15 (d, J=8.0 Hz, 1H), 8.06 (d, J=7.2 Hz, 1H), 7.68 (t, J=7.2 Hz, 1H), 7.63-7.59 (m, 2H), 7.53 (t, J=8.0 Hz, 1H), 7.38 (d, J=9.2 Hz, 1H), 4.61-4.48 (m, 2H), 4.10-3.90 (m, 4H), 3.88-3.75 (m, 1H), 3.65-3.55 (m, 1H), 3.24-3.11 (m, 5H), 2.94 (s, 3H), 2.21-2.16 (m, 1H), 2.04-1.87 (m, 3H). ¹⁹F NMR (376 MHz, DMSO-d₆, ppm): δ −148.71 (1F).

### Example 22 Synthesis of Compound 67

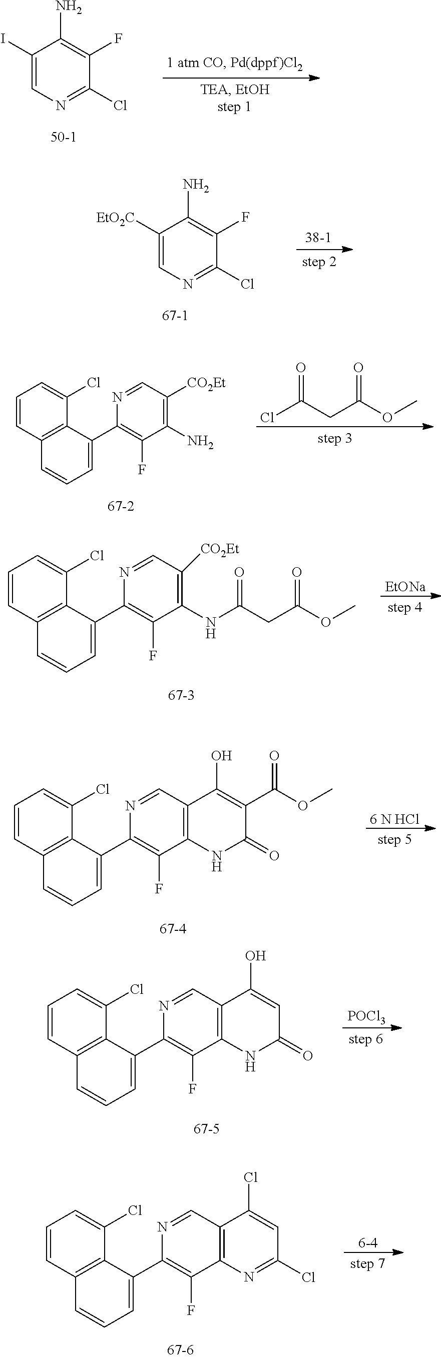

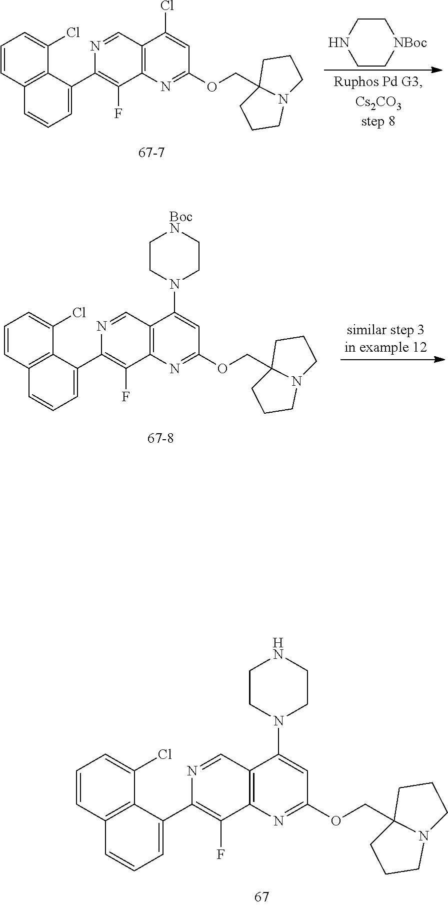 [SMILES: Fc1c(-c2cccc3cccc(Cl)c23)ncc2c(Cl)cc(OCC34CCCN3CCC4)nc12 ; CC(C)(C)OC(=O)N1CCNCC1 ; CC(C)(C)OC(=O)N1CCN(c2cc(OCC34CCCN3CCC4)nc3c(F)c(-c4cccc5cccc(Cl)c45)ncc23)CC1 ; Fc1c(-c2cccc3cccc(Cl)c23)ncc2c(N3CCNCC3)cc(OCC34CCCN3CCC4)nc12]

Step 1: A mixture of 50-1 (15 g, 55.2 mmol), TEA (23 mL, 165.3 mmol) and Pd(dppf)Cl₂ (4 g, 5.52 mmol) in EtOH (150 mL) was stirred at 65° C. for 4 hours under 1 atm of CO atmosphere. After being cooled to room temperature, the mixture was filtered and the filtrate was concentrated. The residue was purified by silica gel column chromatography (petroleum ether/ethyl acetate=10/1) to afford 67-1.

Step 2: A mixture of 67-1 (7.2 g, 33.0 mmol), 38-1 (8.7 g, 42.23 mmol), Pd(PPh₃)₄ (4.0 g, 3.47 mmol) and sodium carbonate (10.9 g, 102.8 mmol) in 1,4-dioxane (200 mL) and water (20 mL) was stirred at 100° C. for 16 hours under nitrogen atmosphere. After being cooled to room temperature, the mixture was diluted with water and extracted with ethyl acetate. The combined organic layers were concentrated and the residue was purified by reverse phase HPLC (acetonitrile with 0.05% of ammonia in water: 50 to 950) to afford 67-2.

Step 3: To a solution of 67-2 (1.2 g, 3.49 mmol) in THF (20 mL) were added DIEA (1.80 g, 3.95 mmol), DMAP (255 mg, 2.09 mmol) and methyl 3-chloro-3-oxopropanoate (1.42 g, 10.44 mmol). The resulting mixture was stirred at reflux for 16 hours. After being cooled to room temperature, water (60 mL) was added and the mixture was extracted with dichloromethane. The combined organic layers were washed with brine, dried over anhydrous sodium sulfate, filtered and concentrated. The residue was purified by prep-TLC (petroleum ether/ethyl acetate=7/3) to afford 67-3.

Step 4: To a solution of 67-3 (900 mg, 1.97 mmol) in ethanol (15 mL) was added EtONa (551 mg, 8.1 mmol). The resulting mixture was stirred at room temperature for 1 hour. The mixture was diluted with water and extracted with ethyl acetate. The combined organic layers were washed with brine, dried over anhydrous sodium sulfate, filtered and concentration to afford 67-4 which was used directly in the next step without purification.

Step 5: A solution of 67-4 (850 mg, 2.06 mmol) in 6 N HCl (12 mL) was stirred at 100° C. for 2 hours. After being cooled to room temperature, the suspension was filtered and the filtered cake was washed with water, and dried to afford 67-5 which was used directly in the next step without purification.

Step 6: A solution of 67-5 (100 mg, 0.29 mmol) in phosphorus oxychloride (3 mL) was stirred at 105° C. for 6 hours. The mixture was concentrated to afford 67-6 which was used directly in the next step without purification.

Step 7: To a solution of 6-4 (300 mg, 2.12 mmol) in THF (7 mL) was added sodium hydride (60% in mineral oil, 85 mg, 2.21 mmol) at 0° C. The resulting mixture was stirred at 0° C. for 50 min. Then a solution of 67-6 (110 mg, 0.29 mmol) in THF (5 mL) was added dropwise at this temperature. The mixture was stirred at 50° C. for 3 hours. After being cooled to room temperature, the mixture was diluted with water and extracted with ethyl acetate. The combined organic layers were washed with brine, dried over anhydrous sodium sulfate, filtered and concentrated. The residue was purified by prep-TLC (petroleum ether/ethyl acetate=2/1) to afford 67-7.

Step 8: A mixture of 67-7 (50 mg, 0.10 mmol), tert-butyl piperazine-1-carboxylate (39 mg, 0.21 mmol), Ruphos Pd G3 (10 mg, 0.013 mmol) and Cs₂CO₃ (102 mg, 0.31 mmol) in 1, 4-dioxane (3 mL) was stirred at 80° C. for 4 hours under N₂ atmosphere. After being cooled to room temperature, the mixture was diluted with water and extracted with ethyl acetate. The combined organic layers were washed with brine, dried over anhydrous sodium sulfate, filtered and concentrated. The residue was purified by prep-TLC (petroleum ether/ethyl acetate=1/1) to afford 67-8.

Compound 67 was prepared from 67-8 following the procedure for the synthesis of compound 35 in example 12 as a 3 eq of TFA salt. LCMS (ESI, m/z): [M+H]⁺=532.3; ¹H NMR (400 MHz, methanol-d₄, ppm): δ 9.10 (s, 1H), 8.14 (d, J=7.6 Hz, 1H), 8.01 (d, J=7.6 Hz, 1H), 7.71-7.66 (m, 1H), 7.61-7.56 (m, 2H), 7.50 (t, J=8.0 Hz, 1H), 6.82 (s, 1H), 4.73 (s, 2H), 3.71-3.57 (m, 10H), 3.30-3.29 (m, 2H), 2.36-2.29 (m, 2H), 2.26-2.07 (m, 6H). ¹⁹F NMR (376 MHz, methanol-d₄, ppm): δ −138.56 (1F).

### Example 23 Synthesis of Compound 69

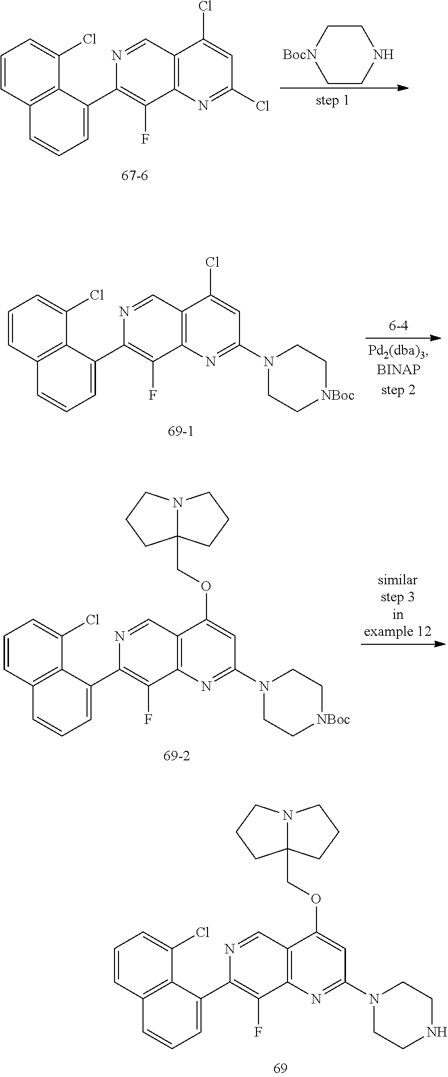 [SMILES: Fc1c(-c2cccc3cccc(Cl)c23)ncc2c(Cl)cc(Cl)nc12 ; CC(C)(C)OC(=O)N1CCNCC1 ; CC(C)(C)OC(=O)N1CCN(c2cc(Cl)c3cnc(-c4cccc5cccc(Cl)c45)c(F)c3n2)CC1 ; CC(C)(C)OC(=O)N1CCN(c2cc(OCC34CCCN3CCC4)c3cnc(-c4cccc5cccc(Cl)c45)c(F)c3n2)CC1 ; Fc1c(-c2cccc3cccc(Cl)c23)ncc2c(OCC34CCCN3CCC4)cc(N3CCNCC3)nc12]

Step 1: To a solution of 67-6 (110 mg, 0.29 mmol) in tetrahydrofuran (6 mL) were added DIEA (318 mg, 2.47 mmol) and tert-butyl piperazine-1-carboxylate (230 mg, 1.24 mmol). The resulting mixture was stirred at 65° C. for 16 hours. Water was added and the mixture was extracted with dichloromethane. The combined organic layers were washed with brine, dried over anhydrous sodium sulfate, filtered and concentrated. The residue was purified by prep-TLC (petroleum ether/ethyl acetate=7/3) to afford 69-1.

Step 2: A mixture of 69-1 (100 mg, 0.19 mmol), 6-4 (54 mg, 0.38 mmol), Pd₂(dba)₃ (17 mg, 0.019 mmol), BINAP (12 mg, 0.019 mmol) and sodium tert-butoxide (73 mg, 0.76 mmol) in toluene (4 mL) was stirred at 95° C. for 3 hours. The mixture was cooled, diluted with water and extracted with ethyl acetate. The combined organic layers were washed with brine, dried over anhydrous sodium sulfate, filtered and concentrated. The residue was purified by prep-HPLC (acetonitrile with 0.05% of TFA in water: 5% to 95%) to afford 69-2.

Compound 69 was prepared from 69-2 following the procedure for the synthesis of compound 35 in example 12 as a 3 eq of TFA salt. LCMS (ESI, m/z): [M+H]⁺=532.3; ¹H NMR (400 MHz, methanol-d₄, ppm): δ 9.02 (s, 1H), 8.22 (d, J=8.0 Hz, 1H), 8.05 (dd, J=8.4, 0.8 Hz, 1H), 7.73 (t, J=7.6 Hz, 1H), 7.69-7.64 (m, 2H), 7.55 (t, J=7.8 Hz, 1H), 6.94 (s, 1H), 4.65 (s, 2H), 4.29-4.25 (m, 4H), 3.80-3.73 (m, 2H), 3.40-3.33 (m, 6H), 2.46-2.39 (m, 2H), 2.32-2.15 (m, 6H). ¹⁹F NMR (376 MHz, methanol-d₄, ppm): δ −137.57 (1F).

### Example 24 Synthesis of Compound 71

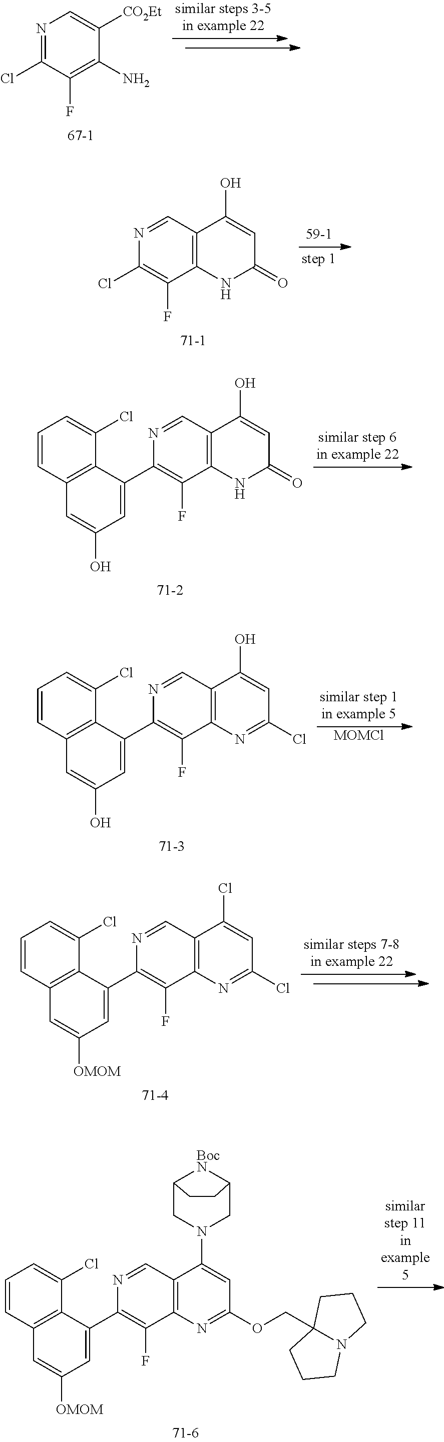

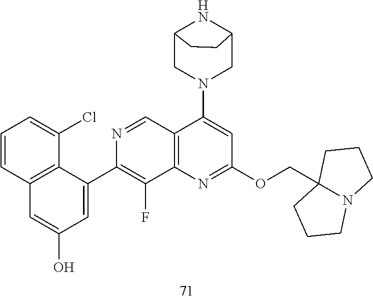 [SMILES: Oc1cc(-c2ncc3c(N4CC5CCC(C4)N5)cc(OCC45CCCN4CCC5)nc3c2F)c2c(Cl)cccc2c1]

Compound 71-1 was prepared from 67-1 following the procedure for the synthesis of compound 67-5 in example 22.

Step 1: A mixture of 71-1 (895 mg, 4.18 mmol), 59-1 (1.16 g, 5.22 mmol), Pd(PPh₃)₄ (483 mg, 0.42 mmol) and Na₂CO₃ (1.33 g, 13.0 mmol) in 1,4-dioxane (34 mL) and water (3.4 mL) was stirred at 130° C. for 2 hours under microwave condition. The mixture was concentrated and the residue was purified by reverse phase HPLC (acetonitrile with 0.05% of TFA in water: 5% to 95%) to afford 71-2.

Compound 71-3 was prepared from 71-2 following the procedure for the synthesis of compound 67-6 in example 22.

Compound 71-4 was prepared from 71-3 following the procedure for the synthesis of compound 60-1 in example 5.

Compound 71-6 was prepared from 71-4 following the procedure for the synthesis of compound 67-8 in example 22.

Compound 71 was prepared from 71-6 following the procedure for the synthesis of compound 60 in example 5 as a 3 eq of TFA salt. LCMS (ESI, m/z): [M+H]⁺=574.3; ¹H NMR (400 MHz, methanol-d₄, ppm): δ 9.13 (s, 1H), 7.77-7.74 (m, 1H), 7.36-7.30 (m, 3H), 7.15 (d, J=2.4 Hz, 1H), 6.80 (s, 1H), 4.73 (s, 2H), 4.29-4.25 (m, 2H), 3.80-3.60 (m, 4H), 3.49-3.39 (m, 3H), 2.49-2.46 (m, 2H), 2.36-2.07 (m, 11H). ¹⁹F NMR (376 MHz, methanol-d₄, ppm): δ −138.37 (1F).

### Example 25 Synthesis of Compound 66

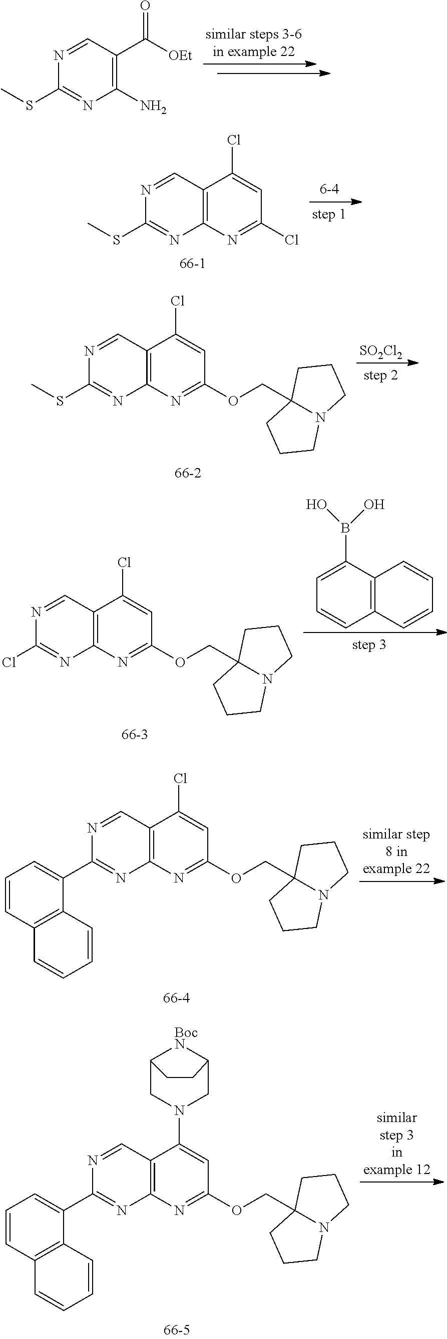

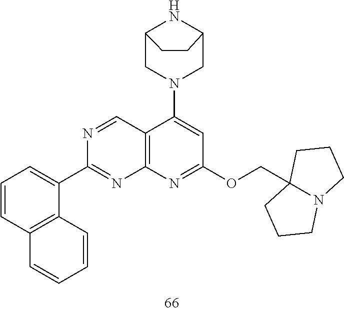 [SMILES: c1ccc2c(-c3ncc4c(N5CC6CCC(C5)N6)cc(OCC56CCCN5CCC6)nc4n3)cccc2c1]

Compound 66-1 was prepared from ethyl 4-amino-2-(methylthio)pyrimidine-5-carboxylate following the procedure for the synthesis of compound 67-6 in example 22.

Step 1: A mixture of 66-1 (620 mg, 2.53 mmol), 6-4 (1.25 g, 8.86 mmol) and Cs₂CO₃ (1.65 g, 5.06 mmol) in THF (30 mL) was stirred at 60° C. for 16 hours. The mixture was cooled, poured into ice-water and extracted with ethyl acetate/tetrahydrofuran (1/1). The combined organic layers were washed with brine, dried over anhydrous sodium sulfate, filtered and concentrated. The residue was purified by reverse phase HPLC (acetonitrile with 0.05% of TFA in water: 5% to 95%) to afford 66-2.

Step 2: To a solution of 66-2 (105 mg, 0.30 mmol) in THF (3 mL) was added dropwise a solution of SO₂Cl₂ (405 mg, 3.0 mmol) in dichloromethane (3 mL). The resulting mixture was stirred at room temperature for 3 hours. The mixture was concentrated and the residue was purified by reverse phase HPLC (acetonitrile with 0.05% of TFA in water: 5% to 95%) to afford 66-3.

Step 3: A mixture of 66-3 (35 mg, 0.10 mmol), 1-naphthylboronic acid (24 mg, 0.14 mmol), Pd(dppf)Cl₂ (12 mg, 0.016 mmol) and Na₂CO₃ (34 mg, 0.045 mmol) in 1,4-dioxane (1.5 mL) and water (0.30 mL) was stirred at 50° C. for 1 hour under microwave condition. The mixture was concentrated and the residue was purified by reverse phase HPLC (acetonitrile with 0.05% of TFA in water: 5% to 95%) to afford 66-4.

Compound 66-5 was prepared from 66-4 following the procedure for the synthesis of compound 67-8 in example 22.

Compound 66 was prepared from 66-5 following the procedure for the synthesis of compound 35 in example 12 as a 3 eq of TFA salt. LCMS (ESI, m/z): [M+H]⁺=507.3; ¹H NMR (400 MHz, methanol-d₄, ppm): δ 9.65 (s, 1H), 8.54 (d, J=7.6 Hz, 1H), 8.12-8.05 (m, 2H), 8.00-7.98 (m, 1H), 7.65 (t, J=8.0 Hz, 1H), 7.59-7.51 (m, 2H), 6.76 (s, 1H), 4.75 (s, 2H), 4.29 (brs, 2H), 3.87-3.84 (m, 2H), 3.72-3.66 (m, 2H), 3.53-3.49 (m, 2H), 2.49-2.46 (m, 2H), 2.35-2.10 (m, 12H).

### Example 26 Synthesis of Compound 81

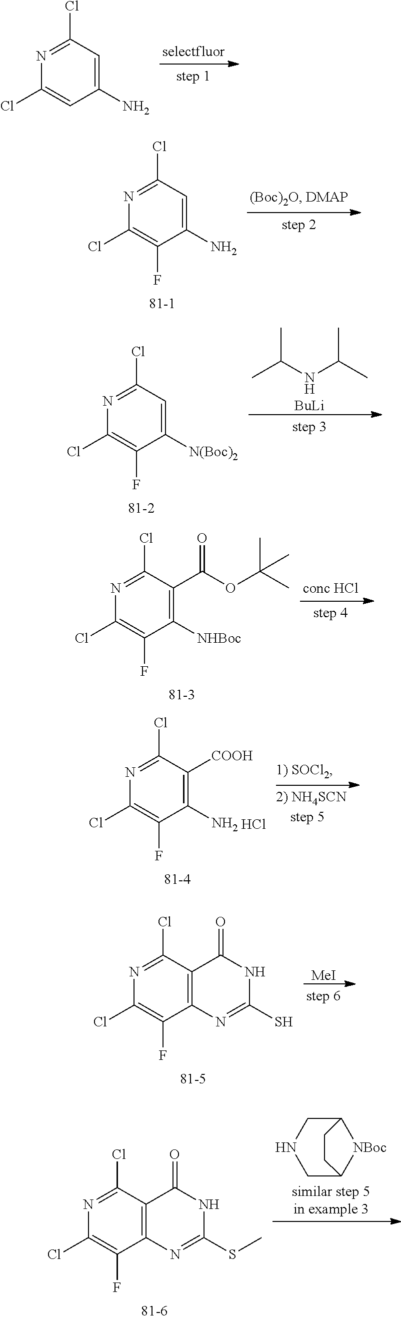

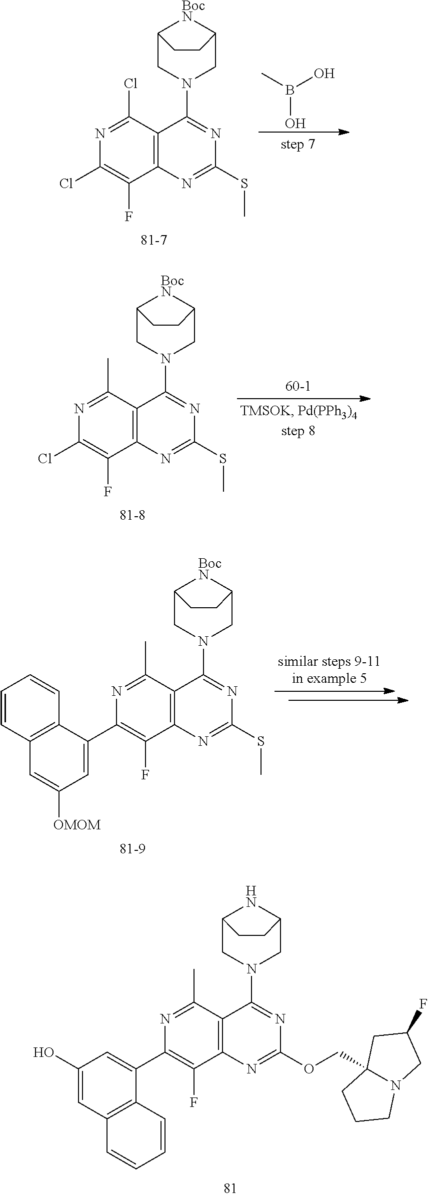

Step 1: To a solution of 2,6-dichloropyridin-4-amine (26 g, 160 mmol) in methanol (250 mL) and water (50 mL) was added selectfluor (68 g, 180 mmol) at room temperature. The mixture was stirred at 45° C. for 16 hours before it was cooled, concentrated, diluted with ethyl acetate and washed with water. The organic layer was dried over sodium sulfate, filtered and concentrated. The residue was purified by column chromatography on silica gel (petroleum ether to petroleum ether/ethyl acetate=1/1) to afford 81-1.

Step 2: To a solution of 81-1 (27.5 g, 153 mmol) in tetrahydrofuran (150 mL) was added 4-dimethylaminopyridine (862 mg, 7.7 mmol) and di-tert-butyl dicarbonate (83.5 g, 383 mmol) at room temperature. The mixture was stirred at 60° C. for 4 hours before it was cooled and concentrated. The residue was triturated with methanol to afford 81-2.

Step 3: To a solution of diisopropylamine (12.16 g, 120.4 mmol) in tetrahydrofuran (100 mL) was added n-butyllithium (75.25 mL, 120.4 mmol) dropwise at −78° C. under N₂ atmosphere. The mixture was stirred at −78° C. for 1 hour before a solution of 81-2 (16.3 g, 43 mmol) in tetrahydrofuran (50 mL) was added at −78° C. under N₂ atmosphere. The resulting mixture was stirred at −78° C. for 1 hour, quenched with acetic acid, diluted with ethyl acetate and washed with water. The organic layer was dried over sodium sulfate, filtered and concentrated. The residue was purified by column chromatography on silica gel (petroleum ether to petroleum ether/ethyl acetate=5/1) to afford 81-3.

Step 4: A mixture of 81-3 (3.8 g, 10 mmol) in dioxane (15 mL) and conc. HCl (5 mL) was stirred at room temperature for 16 hours. The mixture was concentrated to afford 81-4 which was used directly in the next step without purification.

Step 5: 81-4 (2 g, 7.7 mmol) was dissolved in thionyl chloride (50 mL). The mixture was stirred at 50° C. for 3 hours before it was cooled and concentrated. The residue was dissolved in acetone (10 mL). The solution was added into a solution of ammonium thiocyanate (1.76 g, 23 mmol) in acetone (40 mL) dropwise at room temperature. The resulting mixture was stirred at room temperature for 1 hour and diluted with water. The mixture was filtered and the filter cake was washed with water and dried to afford 81-5 which was used directly in the next step without purification.

Step 6: To a solution of 81-5 (crude, from previous step) in methanol (154 mL) was added a solution of sodium hydroxide aqueous (0.1 M, 154 mL, 15.4 mmol) and iodomethane (2.19 g, 15.4 mmol) at room temperature. The mixture was stirred at room temperature for 2 hours before it was poured into water (500 mL) and acidified to pH-6 with conc. HCl. The mixture was filtered, washed with water and dried to afford crude product, which was triturated with acetonitrile to afford 81-6.

Compound 81-7 was prepared from 81-6 following the procedure for the synthesis of compound 10-5 in example 3.

Step 7: A mixture of 81-7 (1.23 g, 2.6 mmol), methylboronic acid (624 mg, 10.4 mmol), [1,1′-bis(diphenylphosphino)ferrocene]dichloropalladium(II) (190 mg, 0.26 mmol) and tripotassium phosphate (1.65 g, 7.8 mmol) in toluene/water (10/1, 11 mL) was stirred at 105° C. for 24 hours under N₂ atmosphere. The mixture was cooled, diluted with ethyl acetate and washed with water. The organic layer was dried over sodium sulfate, filtered and concentrated. The residue was purified by column chromatography on silica gel (petroleum ether to petroleum ether/ethyl acetate=4/1) to afford 81-8.

Step 8: A mixture of 81-8 (182 mg, 0.4 mmol), 60-1 (151 mg, 0.48 mmol), potassium trimethylsilanolate (102 mg, 0.8 mmol) and tetrakis(triphenylphosphine)palladium (46 mg, 0.04 mmol) in 1,4-dioxane (5 mL) was stirred at 95° C. for 0.5 hours under N₂ atmosphere under microwave condition. The mixture was cooled, diluted with ethyl acetate and washed with water. The organic layer was dried over sodium sulfate, filtered and concentrated. The residue was purified by column chromatography on silica gel (petroleum ether to petroleum ether/ethyl acetate=3/1) to 81-9.

Compound 81 was prepared from 81-9 following the procedure for the synthesis of compound 60 in example 5 as a 3 eq of TFA salt. LCMS (ESI, m/z): [M+H]⁺=573.3; ¹H NMR (400 MHz, methanol-d₄, ppm): δ 7.75 (d, J=8.4 Hz, 1H), 7.58-7.55 (m, 1H), 7.44-7.40 (m, 1H), 7.30-7.21 (m, 3H), 5.65-5.49 (m, 1H), 4.86-4.35 (m, 4H), 4.23-4.13 (m, 2H), 4.08-3.62 (m, 5H), 3.50-3.41 (m, 1H), 2.81-2.53 (m, 5H), 2.45-2.31 (m, 3H), 2.29-1.61 (m, 5H). ¹⁹F NMR (376 MHz, methanol-d₄, ppm): δ −114.26 (1F), −174.11 (1F).

### Example 27 Synthesis of Compound 84

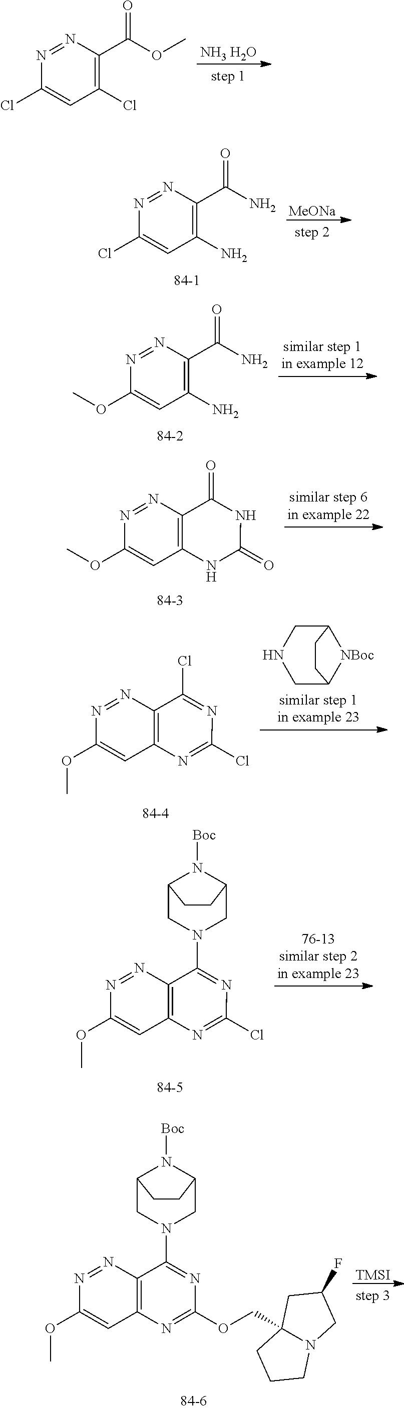

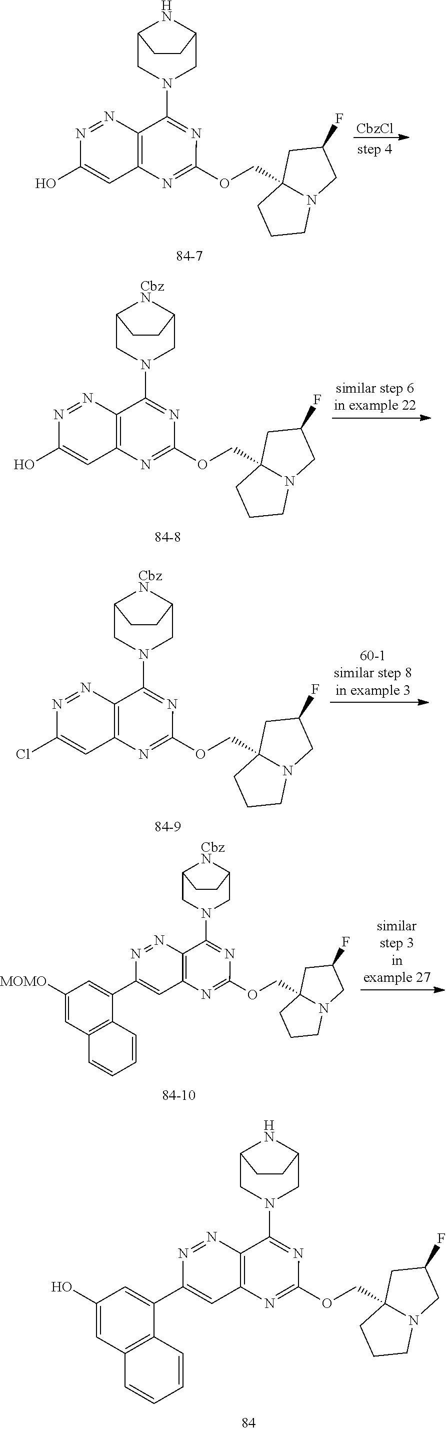

Step 1: To a solution of methyl 4,6-dichloropyridazine-3-carboxylate (16.6 g, 80 mmol) in dioxane (100 mL) was added ammonium (28%, 90 mL) at room temperature. The mixture was stirred at 50° C. for 12 hours in sealed tube. The mixture was cooled, concentrated, diluted with water and stirred for 1 hour. Then, the mixture was filtered and the filter cake was dried to afford 84-1 which was used directly in the next step without purification.

Step 2: A mixture of 84-1 (11 g, 64 mmol) and sodium methoxide (17.3 g, 320 mmol) in methanol (300 mL) was stirred at 95° C. for 10 hours in sealed tube. The mixture was cooled, concentrated, diluted with water and stirred for 1 hour. Then, the mixture was filtered and the filter cake was triturated with acetonitrile to afford 84-2.

Compound 84-3 was prepared from 84-2 following the procedure for the synthesis of compound 35-3 in example 12.

Compound 84-4 was prepared from 84-3 following the procedure for the synthesis of compound 67-6 in example 22.

Compound 84-5 was prepared from 84-4 following the procedure for the synthesis of compound 69-1 in example 23.

Compound 84-6 was prepared from 84-5 and 76-13 following the procedure for the synthesis of compound 69-2 in example 23.

Step 3: To a solution of 84-6 (3.7 g, 7 mmol) in acetonitrile (200 mL) was added iodotrimethylsilane (14 g, 70 mmol) at 0° C. under nitrogen atmosphere. The mixture was stirred at room temperature for 1 hour. Then, the mixture was filtered and the filter cake was washed with acetonitrile and dried to afford 84-7 which was used directly in the next step without purification.

Step 4: 84-7 obtained in previous step was dissolved in dioxane (100 mL) and sat. NaHCO₃ solution (100 mL). To above mixture was added benzyl chloroformate (958 mg, 5.6 mmol) at 0° C. The mixture was stirred at room temperature for 1 hour. The mixture was diluted with ethyl acetate and washed with water. The organic layer was dried over sodium sulfate, filtered and concentrated. The residue was triturated with ethyl acetate to afford 84-8.

Compound 84-9 was prepared from 84-8 following the procedure for the synthesis of compound 67-6 in example 22.

Compound 84-10 was prepared from 84-9 following the coupling procedure for the synthesis of compound 10 in example 3.

Compound 84 was prepared from 84-9 following the procedure for the synthesis of compound 84-7 in example 27 as a 3 eq of TFA salt. LCMS (ESI, m/z): [M+H]⁺=542.3; ¹H NMR (400 MHz, methanol-d₄, ppm): δ 7.89 (s, 1H), 7.78 (d, J=8.4 Hz, 1H), 7.74 (d, J=8.4 Hz, 1H), 7.48-7.43 (m, 1H), 7.33-7.25 (m, 3H), 6.84-6.12 (m, 1H), 5.66-5.03 (m, 2H), 4.75-4.63 (m, 2H), 4.39-4.31 (m, 2H), 4.02-3.58 (m, 5H), 3.52-3.43 (m, 1H), 2.76-2.54 (m, 2H), 2.46-2.30 (m, 3H), 2.27-2.08 (m, 5H). ¹⁹F NMR (376 MHz, methanol-d₄, ppm): δ −174.28 (1F).

### Example 28 Synthesis of Compounds 86, 91 and 92

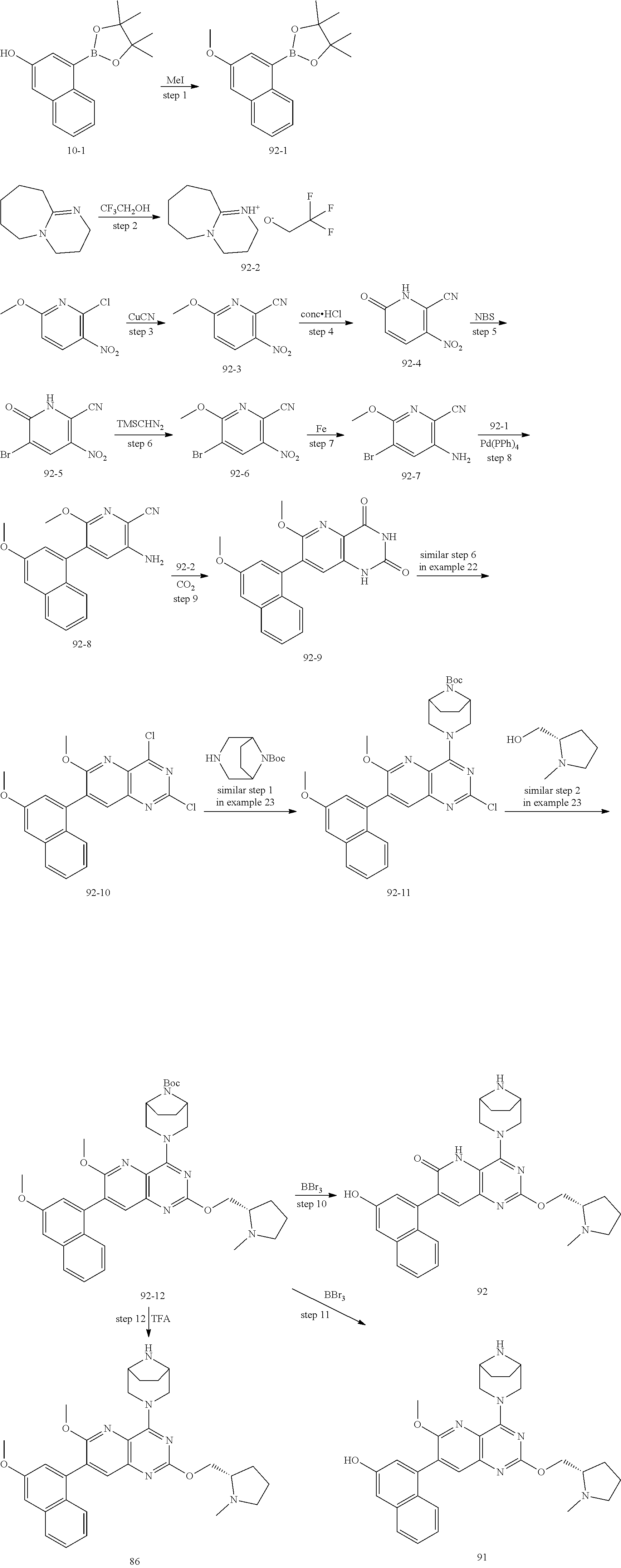

Step 1: To a mixture of 10-1 (9 g, 33.32 mmol) and potassium carbonate (11.5 g, 83.3 mmol) in N, N-dimethylformamide (50 mL) was added iodomethane (7.1 g, 50.0 mmol) at room temperature. The mixture was stirred at room temperature for 4 hours. The mixture was cooled, diluted with ethyl acetate and washed with water. The organic layer was dried over sodium sulfate, filtered and concentrated. The residue was purified by column chromatography on silica gel (petroleum ether to petroleum ether/ethyl acetate=5/1) to afford 92-1.

Step 2: A mixture of 2,3,4,6,7,8,9,10-octahydropyrimido[1,2-a]azepine (33.4 g, 220 mmol) and 2,2,2-trifluoroethanol (23.1 g, 231 mmol) in ethyl acetate (267 mL) was stirred at 25° C. for 72 hours under N₂ atmosphere. The solvent was removed under vacuum to afford 92-2 which was used directly.

Step 3: A mixture of 2-chloro-6-methoxy-3-nitropyridine (56.7 g, 300 mmol) and copper(I) cyanide (40.5 g, 450 mmol) in N, N-dimethylformamide (567 mL) was stirred at 110° C. for 16 hours under N₂ atmosphere. The mixture was diluted with ethyl acetate and washed with water. The organic layer was dried over sodium sulfate, filtered and concentrated. The residue was triturated with petroleum ether to afford 92-3.

Step 4: To a suspension of 92-3 (33.5 g, 186 mmol) in 1,4-dioxane (78 mL) was added concentrated hydrochloric acid (155 mL). The mixture was stirred at 95° C. for 22 hours under N₂ atmosphere. After being cooled to room temperature, the mixture was diluted with ethyl acetate and washed with water. The organic layer was dried over sodium sulfate, filtered and concentrated to afford 92-4 which was used directly in the next step without purification.

Step 5: To a solution of 92-4 (21.9 g, 131.9 mmol) in acetonitrile (132 mL) was added N-bromosuccinimide (28.2 g, 158.3 mmol). The mixture was stirred at 25° C. for 1 hour under N₂ atmosphere. The mixture was diluted with ethyl acetate and washed with water. The organic layer was dried over sodium sulfate, filtered and concentrated. The residue was triturated with petroleum ether/ethyl acetate (9/1) to afford 92-5.

Step 6: To a solution of 92-5 (12.2 g, 50 mmol) in acetonitrile (100 mL) and methanol (17 mL) was added (trimethylsilyl)diazomethane (62.5 mL, 125 mmol, 2M in hexane) at 0° C. over 0.5 hours under N₂ atmosphere. The mixture was quenched with acetic acid and diluted with ethyl acetate and washed with water. The organic layer was dried over sodium sulfate, filtered and concentrated. The residue was purified by column chromatography on silica gel (petroleum ether to petroleum ether/ethyl acetate=1/1) to afford 92-6.

Step 7: To a solution of 92-6 (3.4 g, 13.2 mmol) in acetic acid (68 mL) was added iron powder (3.7 g, 65.9 mmol). The mixture was stirred at 25° C. for 1 hour, then diluted with water and extracted with ethyl acetate. The combined organic layers were washed with water. The organic layer was dried over sodium sulfate, filtered and concentrated to afford 92-7 which was used directly in the next step without purification.

Step 8: A mixture of 92-7 (684 mg, 3 mmol), 92-1 (1.03 g, 3.6 mmol), sodium carbonate (1.27 g, 12 mmol) and tetrakis(triphenylphosphine)palladium (347 mg, 0.3 mmol) in 1,4-dioxane/water (4/1, 45 mL) was stirred at 70° C. for 8 hours under N₂ atmosphere. After being cooled to room temperature, the mixture was diluted with ethyl acetate and washed with water. The organic layer was dried over sodium sulfate, filtered and concentrated. The residue was purified by column chromatography on silica gel (petroleum ether to petroleum ether/ethyl acetate=1/1) to afford 92-8.

Step 9: A mixture of 92-8 (1.64 g, 5.38 mmol) and 92-2 (6.78 g, 26.91 mmol) was stirred at 75° C. for 2 hours under CO₂ atmosphere. After being cooled to room temperature, the mixture was diluted with water. To above mixture was added concentrated hydrochloric acid (2.5 mL, 29.6 mmol). The precipitate was filtered and collected to afford 92-9 which was used directly in the next step without purification.

Compound 92-10 was prepared from 92-9 following the procedure for the synthesis of compound 67-6 in example 22.

Compound 92-11 was prepared from 92-10 following the procedure for the synthesis of compound 69-1 in example 23.

Compound 92-12 was prepared from 92-11 following the procedure for the synthesis of compound 69-2 in example 23.

Step 10: To s solution of 92-12 (128 mg, 0.2 mmol) in dichloromethane (2 mL) was added boron tribromide (10 mL, 10 mmol, 1.0M in dichloromethane) at 0° C. The mixture was stirred at 25° C. for 20 hours under N₂ atmosphere. The mixture was filtered and washed with dichloromethane. The filtrate cake was dissolved in acetonitrile/water (1/1) and then purified by prep-HPLC (acetonitrile with 0.05% of TFA in water: 5% to 95%) to afford 92. LCMS (ESI, m/z): [M+H]⁺=513.3; ¹H NMR (400 MHz, methanol-d₄, ppm): δ 7.84 (s, 1H), 7.71 (d, J=8.8 Hz, 1H), 7.40-7.37 (m, 2H), 7.22-7.17 (m, 2H), 7.06 (d, J=2.0 Hz, 1H), 6.31-5.52 (m, 2H), 4.81-4.75 (m, 1H), 4.68-4.63 (m, 1H), 4.28-4.23 (m, 2H), 3.96-3.85 (m, 1H), 3.78-3.60 (m, 3H), 3.24-3.22 (m, 1H), 3.07 (s, 3H), 2.43-2.36 (m, 1H), 2.19-2.00 (m, 7H).

Step 11: To s solution of 92-12 (128 mg, 0.2 mmol) in dichloromethane (1.6 mL) was added boron tribromide (1.6 mL, 1.6 mmol, 1.0M in dichloromethane) at 0° C. The mixture was stirred at 25° C. for 2 hours under N₂ atmosphere. The mixture was filtered and washed with dichloromethane. The filtrate cake was dissolved in acetonitrile/water (1/1) and then purified by prep-HPLC (acetonitrile with 0.05% of TFA in water: 5% to 95%) to afford 91. LCMS (ESI, m/z): [M+H]⁺=527.3; ¹H NMR (400 MHz, methanol-d₄, ppm): δ 7.86 (s, 1H), 7.71 (d, J=8.4 Hz, 1H), 7.38 (t, J=7.6 Hz, 1H), 7.29 (d, J=8.4 Hz, 1H), 7.21-7.16 (m, 2H), 7.02 (d, J=2.0 Hz, 1H), 5.98-5.59 (m, 2H), 4.82-4.81 (m, 1H), 4.65-4.60 (m, 1H), 4.34-4.25 (m, 2H), 3.94-3.84 (m, 4H), 3.78-3.63 (m, 3H), 3.25-3.22 (m, 1H), 3.08 (s, 3H), 2.42-2.36 (m, 1H), 2.24-2.00 (m, 7H).

Step 12: To s solution of 92-12 (100 mg, 0.16 mmol) in dichloromethane (2 mL) was added trifluoroacetic acid (0.5 mL). The mixture was stirred at 25° C. for 1 hour under N₂ atmosphere. The mixture was concentrated and the residue was purified by prep-HPLC (acetonitrile with 0.05% of TFA in water: 5% to 95%) to afford 86. LCMS (ESI, m/z): [M+H]⁺=541.3; ¹H NMR (400 MHz, methanol-d₄, ppm): δ 7.87-7.83 (m, 2H), 7.44 (t, J=7.6 Hz, 1H), 7.35-7.31 (m, 2H), 7.24 (t, J=7.6 Hz, 1H), 7.08 (d, J=2.0 Hz, 1H), 6.01-5.58 (m, 2H), 4.85-4.81 (m, 1H), 4.67-4.62 (m, 1H), 4.33-4.27 (m, 2H), 3.94-3.88 (m, 7H), 3.78-3.66 (m, 3H), 3.24-3.22 (m, 1H), 3.07 (s, 3H), 2.42-2.36 (m, 1H), 2.23-2.00 (m, 7H).

### Example 29 Synthesis of Compound 96

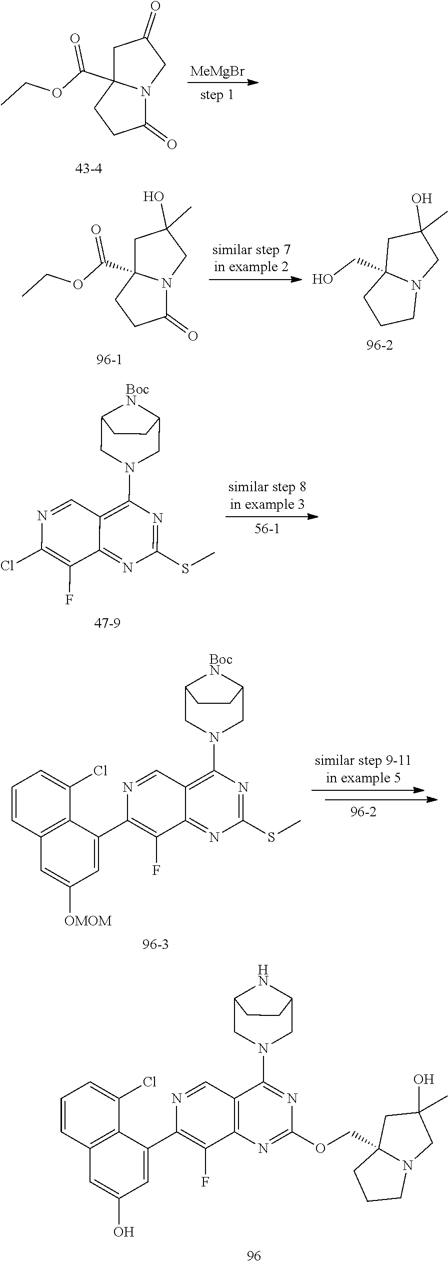

Step 1: To a solution of 43-4 (8 g, 37.9 mmol) in tetrahydrofuran (200 mL) was added methylmagnesium bromide (17.7 mL, 3.0 M in THF, 53 mmol) at −60° C. under N₂ atmosphere. The mixture was stirred at −60° C. for 2 hours. The mixture was quenched with saturated ammonium chloride solution and extracted with dichloromethane. The combined organic layers were concentrated. The residue was purified by column chromatography on silica gel (petroleum ether to ethyl acetate) to afford 96-1.

Compound 96-2 was prepared from 96-1 following the procedure for the synthesis of compound 43 in example 2.

Compound 96-3 was prepared from 47-9 and 56-1 following the coupling procedure for the synthesis of compound 10 in example 3.

Compound 96 was prepared from 96-3 following the procedure for the synthesis of compound 60 in example 5 as a 3 eq of TFA salt. LCMS (ESI, m/z): [M+H]⁺=605.3; ¹H NMR (400 MHz, methanol-d₄, ppm): δ 9.09 (s, 1H), 7.75 (dd, J=8.0, 1.2 Hz, 1H), 7.39-7.31 (m, 3H), 7.14 (dd, J=2.4, 1.2 Hz, 1H), 5.10-5.06 (m, 1H), 5.02-4.90 (m, 1H), 4.80-4.66 (m, 2H), 4.28-4.21 (m, 2H), 4.01-3.91 (m, 2H), 3.63-3.50 (m, 2H), 3.40-3.33 (m, 1H), 3.19-3.15 (m, 1H), 2.52-2.01 (m, 10H), 1.43 (s, 3H). ¹⁹F NMR (376 MHz, methanol-d₄, ppm): δ −139.35 (1F).

### Example 30 Synthesis of Compound 106

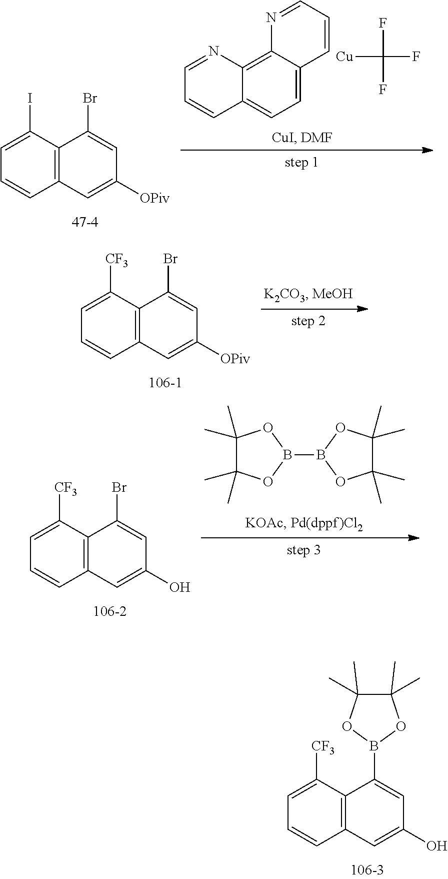

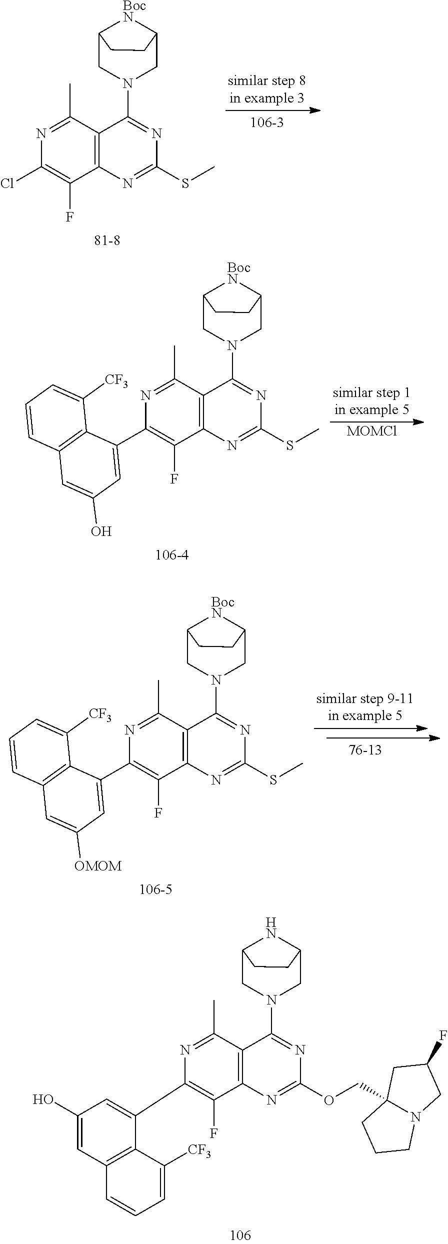

Step 1: A mixture of 47-4 (1.8 g, 4.15 mmol), CuI (473 mg, 2.5 mmol) and (1,10-phenanthroline)(trifluoromethyl)copper(I) (1.95 g, 6.23 mmol) in DMF (10 mL) was stirred at room temperature for 1 hour under N₂. The mixture was filtered and rinsed with ethyl acetate. The filtrate was washed with water and brine, dried over sodium sulfate, filtered and concentrated. The residue was purified by column chromatography on silica gel (petroleum ether to petroleum ether/ethyl acetate=10/1) to afford 106-1.

Step 2: A mixture of 106-1 (1.11 g, 2.96 mmol) and potassium carbonate (571 mg, 4.14 mmol) in methanol (15 mL) was stirred at room temperature for 1 hour. Then the mixture was diluted with dichloromethane and adjusted to around pH 6 with 2N hydrochloric acid. The organic layer was washed with water and brine, dried over sodium sulfate, filtered and concentrated. The residue was purified by column chromatography on silica gel (petroleum ether to petroleum ether/ethyl acetate=4/1) to afford 106-2.

Step 3: A mixture of 106-2 (830 mg, 2.85 mmol), bis(pinacolato)diboron (1.45 g, 5.7 mmol), potassium acetate (838 mg, 8.55 mmol) and [1,1′-bis(diphenylphosphino)ferrocene]dichloropalladium(II) (212 mg, 0.29 mmol) in dioxane (20 mL) was stirred at 95° C. for 2 hours under N₂ atmosphere. Then cooled, filtered, diluted with water and extracted with ethyl acetate. The combined organic layers were washed with brine, dried over sodium sulfate, filtered and concentrated. The residue was purified by column chromatography on silica gel (petroleum ether to petroleum ether/ethyl acetate=4/1) to afford 106-3.

Compound 106-4 was prepared from 81-8 and 106-3 following the coupling procedure for the synthesis of compound 10 in example 3.

Compound 106-5 was prepared from 106-4 following the procedure for the synthesis of compound 60-1 in example 5.

Compound 106 was prepared from 106-5 following the procedure for the synthesis of compound 60 in example 5 as a 4 eq of TFA salt. LCMS (ESI, m/z): [M+H]⁺=641.3; ¹H NMR (400 MHz, methanol-d₄, ppm): δ 8.04 (d, J=8.0 Hz, 1H), 7.77 (d, J=7.2 Hz, 1H), 7.53 (t, J=8.0 Hz, 1H), 7.41 (d, J=2.8 Hz, 1H), 7.27-7.19 (m, 1H), 5.64-5.48 (m, 1H), 4.81-3.79 (m, 11H), 3.52-3.41 (m, 1H), 2.83-1.57 (m, 13H). ¹⁹F NMR (376 MHz, methanol-d₄, ppm): δ −56.96 (3F), −144.55 (1F), −174.10 (1F).

### Example 31 Synthesis of Compound 103

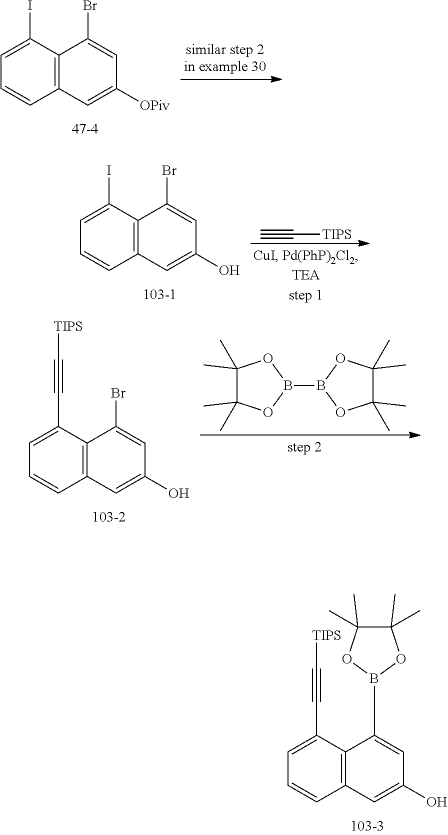

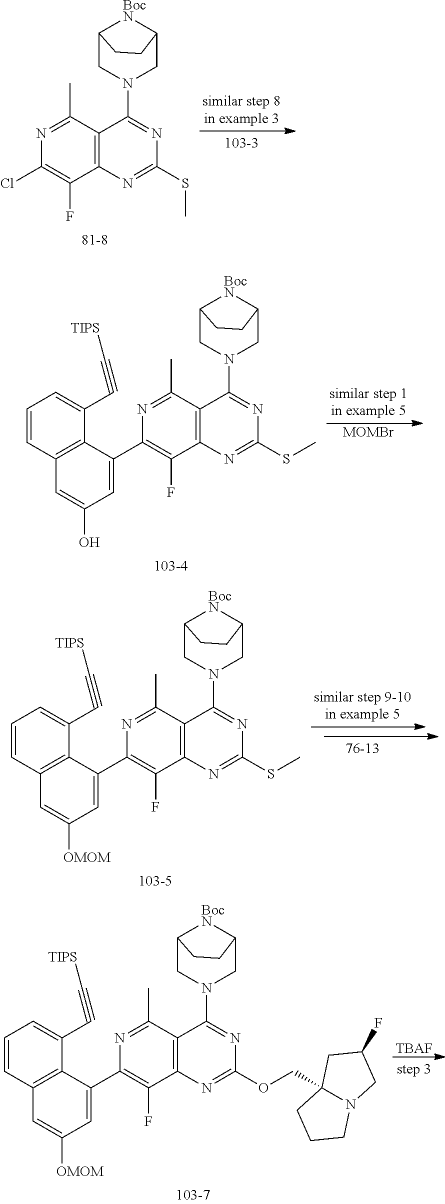

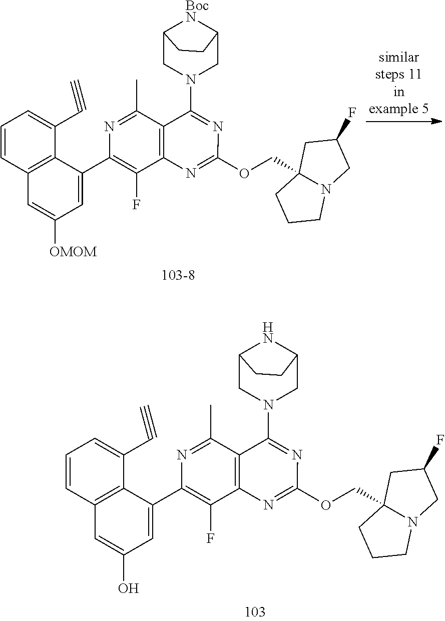

Compound 103-1 was prepared from 47-4 following the procedure for the synthesis of compound 106-2 in example 30.

Step 1: A mixture of 103-1 (4.0 g, crude, from previous step), ethynyltriisopropylsilane (3.13 g, 17.19 mmol) and cuprous iodide (218 mg, 1.15 mmol) in triethylamine (46 mL) was stirred at 25° C. for 10 hours under N₂ atmosphere. The mixture was diluted with ethyl acetate, washed with water and brine, dried over sodium sulfate, filtered and concentrated. The residue was purified by column chromatography on silica gel (petroleum ether to petroleum ether/ethyl acetate=1/1) to afford 103-2.

Step 2: A mixture of 103-2 (3.6 g, 8.9 mmol), bis(pinacolato)diboron (4.54 g, 17.9 mmol), potassium acetate (2.65 g, 26.8 mmol) and [1,1′-bis(diphenylphosphino)ferrocene]dichloropalladium(II) (654 mg, 0.9 mmol) in 1,4-dioxane (36 mL) was stirred at 95° C. for 2 hours under N₂ atmosphere. The mixture was cooled, diluted with ethyl acetate, washed with water and brine, dried over sodium sulfate, filtered and concentrated. The residue was purified by flash column chromatograph (petroleum ether to petroleum ether/ethyl acetate=1/1) to afford 103-3.

Compound 103-4 was prepared from 81-8 and 103-3 following the coupling procedure for the synthesis of compound 10 in example 3.

Compound 103-5 was prepared from 103-4 following the procedure for the synthesis of compound 60-1 in example 5.

Compound 103-7 was prepared from 103-5 following the procedure for the synthesis of compound 60-10 in example 5.

Step 3: To a solution of 103-7 (60 mg, 0.067 mmol) in tetrahydrofuran (0.5 mL) was added tetrabutylammonium fluoride (0.334 mL, 0.334 mmol) at 25° C. The mixture was stirred for 30 minutes and diluted with ethyl acetate (80 mL), washed with water (80 mL), brine (80 mL), dried over sodium sulfate, filtered and concentrated. The residue was purified by prep-HPLC (acetonitrile with 0.05% of TFA in water: 5% to 95%) to afford 103-8.

Compound 103 was prepared from 103-8 following the procedure for the synthesis of compound 60 in example 5 as a 3 eq of TFA salt. LCMS (ESI, m/z): [M+H]⁺=597.3; ¹H NMR (400 MHz, methanol-d₄, ppm): δ 7.81 (d, J=8.4 Hz, 1H), 7.50 (d, J=6.8 Hz, 1H), 7.38 (t, J=7.6 Hz, 1H), 7.32 (d, J=2.4 Hz, 1H), 7.16-7.13 (m, 1H), 5.62-5.49 (m, 1H), 4.70-4.69 (m, 2H), 4.64-4.35 (m, 2H), 4.24-4.11 (m, 2H), 4.06-3.80 (m, 5H), 3.48-3.41 (m, 1H), 2.95-2.92 (m, 1H), 2.78-2.53 (m, 5H), 2.45-2.29 (m, 3H), 2.24-1.75 (m, 5H).

### Example 32 Synthesis of Compound 98

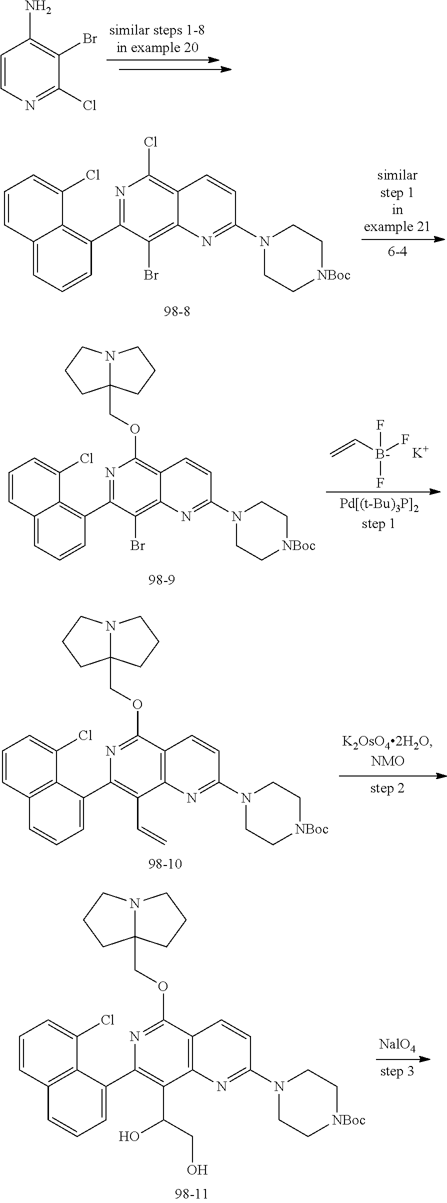

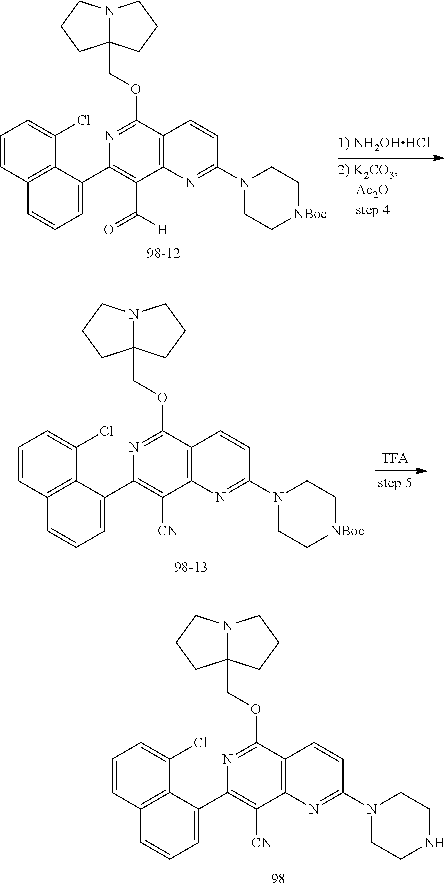 [SMILES: CC(C)(C)OC(=O)N1CCN(c2ccc3c(OCC45CCCN4CCC5)nc(-c4cccc5cccc(Cl)c45)c(C=O)c3n2)CC1 ; CC(C)(C)OC(=O)N1CCN(c2ccc3c(OCC45CCCN4CCC5)nc(-c4cccc5cccc(Cl)c45)c(C#N)c3n2)CC1 ; N#Cc1c(-c2cccc3cccc(Cl)c23)nc(OCC23CCCN2CCC3)c2ccc(N3CCNCC3)nc12]

Compound 98-8 was prepared from 3-bromo-2-chloropyridin-4-amine following the procedure for the synthesis of compound 50-8 in example 20.

Compound 98-9 was prepared from 98-8 following the procedure for the synthesis of compound 53-1 in example 21.

Step 1: A mixture of 53-1 (65 mg, 0.094 mmol), Pd((t-Bu)₃P)₂ (5 mg, 0.0094 mmol), potassium trifluoro(vinyl)borate (19 mg, 0.14 mmol) and sodium carbonate (30 mg, 0.28 mmol) in 1, 4-dioxane (2 mL) and water (0.2 mL) was stirred at 100° C. for 2 hours under N₂ atmosphere. The mixture was concentrated. The residue was purified by reverse phase HPLC (acetonitrile with 0.05% of TFA in water: 5% to 95%) to afford 98-10.

Step 2: To a solution of 98-10 (55 mg, 0.086 mmol) in THF (1 mL), t-BuOH (0.5 mL) and water (0.5 mL) were added potassium osmate (VI) dihydrate (3 mg, 0.0086 mmol) and NMO (25 mg, 0.21 mmol). The resulting mixture was stirred at room temperature for 2 hours and concentrated to afford 98-11 which was used directly in the next step without purification.

Step 3: 98-11 was dissolved in acetone (1 mL) and water (1 mL) and to this mixture was added sodium periodate (64 mg, 0.13 mmol). The resulting mixture was stirred at room temperature for 2 hours. The mixture was concentrated. The residue was purified by reverse phase HPLC (acetonitrile with 0.05% of TFA in water: 5% to 95%) to afford 98-12.

Step 4: A mixture of 98-12 (21 mg, 0.030 mmol) and hydroxylamine hydrochloride (2.7 mg, 0.040 mmol) in DMSO (1 mL) was stirred at 90° C. for 1 hour. The mixture was cooled. To this mixture were added potassium carbonate (18 mg, 0.14 mmol) and acetic anhydride (14 mg, 0.14 mmol). The resulting mixture was stirred at 50° C. for 48 hours. The mixture was cooled, filtered and concentrated. The residue was purified by reverse phase HPLC (acetonitrile with 0.05% of TFA in water: 5% to 95%) to afford 98-13.

Step 5: To a solution of 98-13 (10 mg, 0.016 mmol) in DCM (1.5 mL) was added TFA (0.5 mL) and the mixture was stirred at room temperature for 1 hour. The mixture was concentrated. The residue was purified by prep-HPLC (acetonitrile with 0.05% of TFA in water: 5% to 95%) to afford 98 as a 3 eq of TFA salt. LCMS (ESI, m/z): [M+H]⁺=539.3; ¹H NMR (400 MHz, Methanol-d₄, ppm): δ 8.43 (d, J=9.4 Hz, 1H), 8.15-8.11 (m, 1H), 8.04-7.98 (m, 1H), 7.68-7.64 (m, 1H), 7.63-7.58 (m, 2H), 7.54-7.47 (m, 1H), 7.40 (d, J=9.4 Hz, 1H), 4.65 (s, 2H), 4.28-4.14 (m, 4H), 3.67-3.54 (m, 2H), 3.43-3.36 (m, 4H), 3.26-3.20 (m, 2H), 2.34-2.15 (m, 4H), 2.15-2.00 (m, 4H).

### Example 33 Synthesis of Compound 102

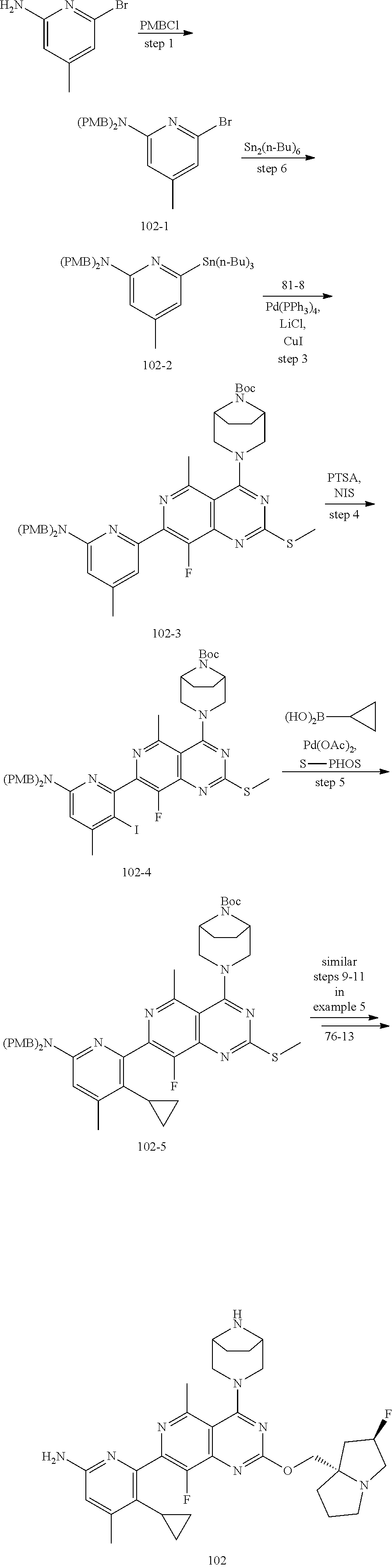

Step 1: To a solution of 6-bromo-4-methylpyridin-2-amine (10 g, 53 mmol) in DMF (150 mL) was added in portions 60% wt. NaH in mineral oil (8.13 g, 203 mmol) at 0° C. The resulting mixture was stirred at room temperature for 1 hour. Then 4-methoxybenzylchloride (18.3 g, 117 mmol) was added to the above reaction and the mixture was stirred at this temperature for 2 hours. After being quenched with saturated NH₄Cl solution, the mixture was extracted with ethyl acetate. The combined organic layers were washed with brine, dried over anhydrous Na₂SO₄, filtered and concentrated. The residue was purified by silica gel column chromatography (petroleum ether to petroleum ether/ethyl acetate=1/10) to afford 102-1.

Step 2: A mixture of 102-1 (1 g, 2.3 mmol), hexabutylditin (4.1 g, 7.1 mmol), Pd₂(dba)₃ (215 mg, 0.23 mmol), tricyclohexyl phosphine (131 mg, 0.46 mmol) and lithium chloride (492 mg, 11.7 mmol) in 1,4-dioxane (20 mL) was stirred at 110° C. for 5 hours under nitrogen atmosphere. The reaction mixture was concentrated and the residue was purified by flash chromatography on silica gel (petroleum ether/ethyl acetate=10/1) to afford 102-2.

Step 3: A mixture of 102-2 (562 mg, 0.88 mmol), 81-8 (200 mg, 0.44 mmol), Pd₂(dba)₃ (254 mg, 0.22 mmol), LiCl (74 mg, 1.7 mmol) and CuI (84 mg, 0.44 mmol) in DMAc (4 mL) was stirred at 120° C. for 3 hours under N₂ atmosphere. After being cooled to room temperature, the mixture was diluted with ethyl acetate, washed with water and brine. The combined organic layers were dried over sodium sulfate, filtered and concentrated. The residue was purified by column chromatography on silica gel (petroleum ether/ethyl acetate=5/1) to afford 102-3.

Step 4: A mixture of 102-3 (250 mg, 0.33 mmol), NIS (367 mg, 1.6 mmol) and PTSA (6 mg, 0.030 mmol) in DMF (5 mL) was stirred at room temperature for 5 hours. After being diluted with water, the mixture was extracted with ethyl acetate. The combined organic layers were washed with brine, dried over sodium sulfate, filtered and concentrated. The residue was purified by column chromatography on silica gel (petroleum ether/ethyl acetate=4/1) to afford 102-4.

Step 5: A mixture of 102-4 (150 mg, 0.17 mmol), cyclopropylboronic acid (72 mg, 0.84 mmol), Pd(OAc)₂ (4 mg, 0.017 mmol), S—PHOS (14 mg, 0.033 mmol) and K₃PO₄ (107 mg, 0.5 mmol) in toluene (3 mL) and water (0.3 mL) was stirred at 105° C. for 3 hours under N₂ atmosphere. After being cooled to room temperature, the mixture was diluted with ethyl acetate, washed with water and brine. The organic layer was dried over sodium sulfate, filtered and concentrated. The residue was purified by column chromatography on silica gel (petroleum ether/ethyl acetate=5/1) to afford 102-5.

Compound 102 was prepared from 102-5 following the procedure for the synthesis of compound 60 in example 5 as a 4 eq of TFA salt. LCMS (ESI, m/z): [M+H]⁺=577.3; ¹H NMR (400 MHz, methanol-d₄, ppm): δ 6.94 (s, 1H), 5.68-5.48 (m, 1H), 4.76-4.63 (m, 2H), 4.51 (brs, 1H), 4.15 (s, 2H), 4.06-3.77 (m, 5H), 3.55-3.38 (m, 1H), 2.82-2.66 (m, 4H), 2.65-2.55 (m, 4H), 2.55-2.26 (m, 4H), 2.25-1.67 (m, 6H), 0.80 (brs, 2H), 0.08 (brs, 2H). ¹⁹F NMR (376 MHz, methanol-d₄, ppm): δ −139.25 (1F), −173.13 (1F).

### Example 34 Synthesis of Compound 107

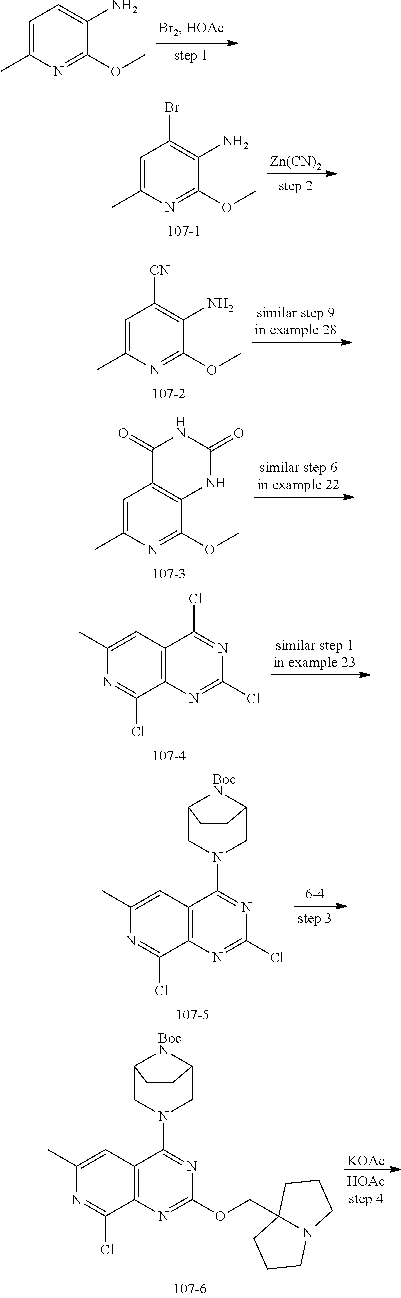

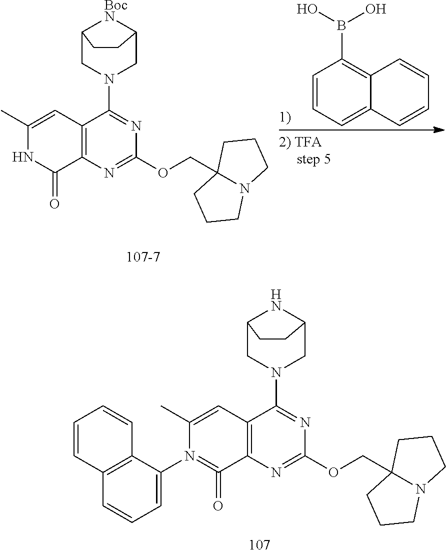

Step 1: To a solution of 2-methoxy-6-methyl-pyridin-3-amine (12.3 g, 89.02 mmol) in DCM (130 mL) and methanol (26 mL) were added HOAc (10.69 g, 178.05 mmol) and bromine (17.07 g, 106.83 mmol) at 0° C. The mixture stirred at 25° C. for 2 hours. The mixture was poured into H₂O and extracted with dichloromethane. The organic layer was dried over sodium sulfate, filtered and concentrated. The residue was purified by silica gel column chromatography (dichloromethane/methanol=10/1) to afford 107-1.

Step 2: To a solution of 107-1 (5.3 g, 24.42 mmol) in DMA (25 mL) were added zinc powder (15.87 g, 244.17 mmol), Zn(CN)₂ (14.34 g, 122.08 mmol), dppf (5.41 g, 9.77 mmol) and Pd₂(dba)₃ (4.47 g, 4.88 mmol). The mixture stirred at 130° C. for 4 hours under N₂. The mixture was poured into H₂O and extracted with ethyl acetate. The organic layer was dried over sodium sulfate, filtered and concentrated. The residue was purified by silica gel column chromatography (dichloromethane/methanol=20/1) to afford 107-2.

Compound 107-3 was prepared from 107-2 following the procedure for the synthesis of compound 92-9 in example 28.

Compound 107-4 was prepared from 107-3 following the procedure for the synthesis of compound 67-7 in example 22.

Compound 107-5 was prepared from 107-4 following the procedure for the synthesis of compound 69-1 in example 23.

Step 3: To a solution of 107-5 (1.7 g, 4.02 mmol) in DMF (10 mL) and THE (10 mL) were added 1,4-diazabicyclo[2.2.2]octane (450.96 mg, 4.02 mmol), Cs₂CO₃ (2.62 g, 8.04 mmol) and 6-4 (850.95 mg, 6.03 mmol). The mixture stirred at 25° C. for 12 hours. The mixture was poured into H₂O and extracted with ethyl acetate. The organic layer was dried over sodium sulfate, filtered and concentrated. The residue was purified by reversed phase HPLC (acetonitrile with 0.05% of TFA in water: 5% to 95%) to afford 107-6.

Step 4: To a solution of 107-6 (1.1 g, 2.08 mmol) in HOAc (50 mL) was added KOAc (612.13 mg, 6.24 mmol). The mixture was stirred at 80° C. for 20 hours. The mixture was concentrated and the residue was diluted with water. The water layer was adjusted to around pH=8 with saturated aqueous NaHCO₃ and then extracted with ethyl acetate. The organic layer was dried over sodium sulfate, filtered and concentrated to afford 107-7 which was used directly in the next step without purification.

Step 5: To a solution of 107-7 (50 mg, 0.10 mmol) in MeCN (3 mL) were added naphthalen-1-yl-boronic acid (35 mg, 0.20 mmol), pyridine (32 mg, 0.40 mmol) and Cu(OAc)₂ (40 mg, 0.20 mmol). The mixture was stirred at 60° C. for 24 hours under O₂. The mixture was cooled and TFA (1 mL) added. The resulting mixture was stirred for 1 hour and then concentrated. The residue was purified by prep-HPLC (acetonitrile with 0.05% of TFA in water: 5% to 95%) to afford 107 as a 2 eq of TFA salt. LCMS (ESI, m/z): [M+H]⁺=537.3; ¹H NMR (400 MHz, methanol-d₄, ppm): δ 7.96 (d, J=8 Hz, 1H), 7.89 (d, J=8.4 Hz, 1H), 7.82 (d, J=8.4 Hz, 1H), 7.55-7.51 (m, 2H), 7.47-7.45 (m, 1H), 7.35 (s, 1H), 7.26 (d, J=7.2 Hz, 1H), 4.71 (d, J=13.2 Hz, 2H), 4.53 (s, 2H), 4.23 (s, 2H), 3.82 (d, J=14 Hz, 2H), 3.65-3.62 (m, 2H), 3.15-3.10 (m, 2H), 2.30 (s, 3H), 2.23-2.02 (m, 12H).

### Example 35 Synthesis of Compound 108

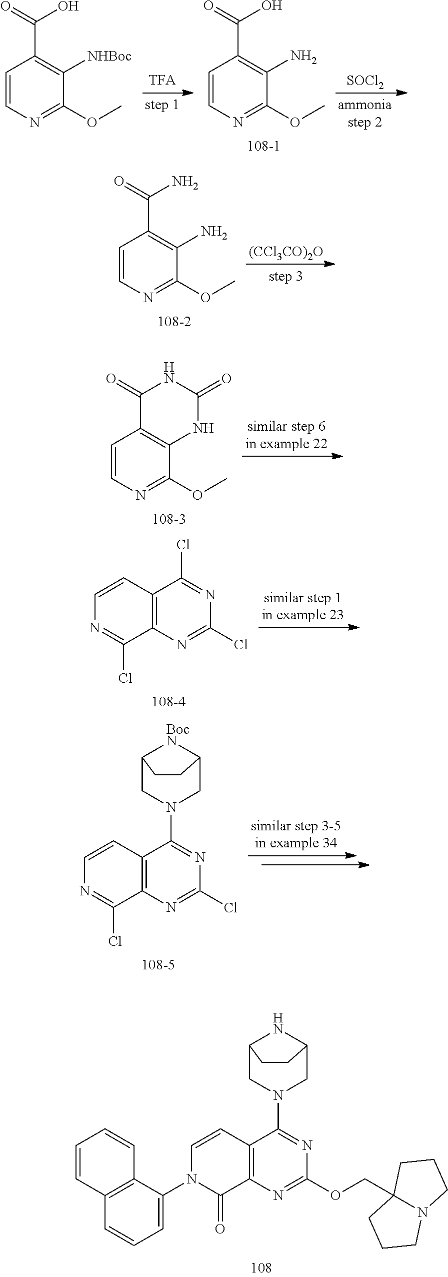

Step 1: To a solution of 3-(tert-butoxycarbonylamino)-2-methoxy-pyridine-4-carboxylic acid (5.0 g, 18.64 umol) in chloroform (20.0 mL) and methanol (20.0 mL) was added trifluoroacetic acid (40.0 mL) at 0° C. under nitrogen atmosphere. Then the solution was allowed to warm to 25° C. and stirred for 48 hours. The solution was concentrated to afford 108-1 which used in the next step without further purification.

Step 2: A solution of 108-1 (3.2 g, 19.04 mmol) in thionyl chloride (30.0 mL) was stirred at 80° C. for 2 hours. Then concentrated and the residue was dissolved in anhydrous dichloromethane (30.0 mL). The mixture was added to a mixture of ammonia (25%, 30.0 mL) and dichloromethane (30.0 mL) at 0° C. After addition, the mixture was warmed to room temperature and stirred for 2 hours. The mixture was concentrated. The residue was diluted with water extracted with ethyl acetate. The combined organic layers were washed with brine, dried over anhydrous sodium sulfate, filtered and the filtrate was concentrated to afford 108-2 which was used directly in the next step without purification.

Step 3: To a solution of 108-2 (1.0 g, 5.94 mmol) in anhydrous tetrahydrofuran (40.0 mL) was added triphosgene (1.76 g, 5.94 mmol) at room temperature under nitrogen atmosphere. Then the mixture was allowed to warm to 70° C. and stirred for 4 hours. The mixture was cooled and concentrated. Ethyl acetate was added and the resulting precipitate was collected by filtration to afford 108-3 which was used directly in the next step without purification.

Compound 108-4 was prepared from 108-3 following the procedure for the synthesis of compound 67-7 in example 22.

Compound 108-5 was prepared from 108-4 following the procedure for the synthesis of compound 69-1 in example 23.

Compound 108 was prepared from 108-5 following the procedure for the synthesis of compound 107 in example 34. LCMS (ESI, m/z): [M+H]⁺=523.3; ¹H NMR (400 MHz, methanol-d₄, ppm): δ 7.97 (d, J=8.4 Hz, 1H), 7.85 (d, J=8 Hz, 2H), 7.78 (d, J=6 Hz, 1H), 7.57-7.51 (m, 3H), 7.46-7.44 (m, 1H), 7.30 (d, J=7.2 Hz, 1H), 4.64 (s, 2H), 3.83 (d, J=14.4 Hz, 2H), 3.68-3.65 (m, 2H), 3.47-3.46 (m, 4H), 3.12-3.10 (m, 2H), 2.27-2.02 (m, 12H).

### Example 36 Synthesis of Compound 109

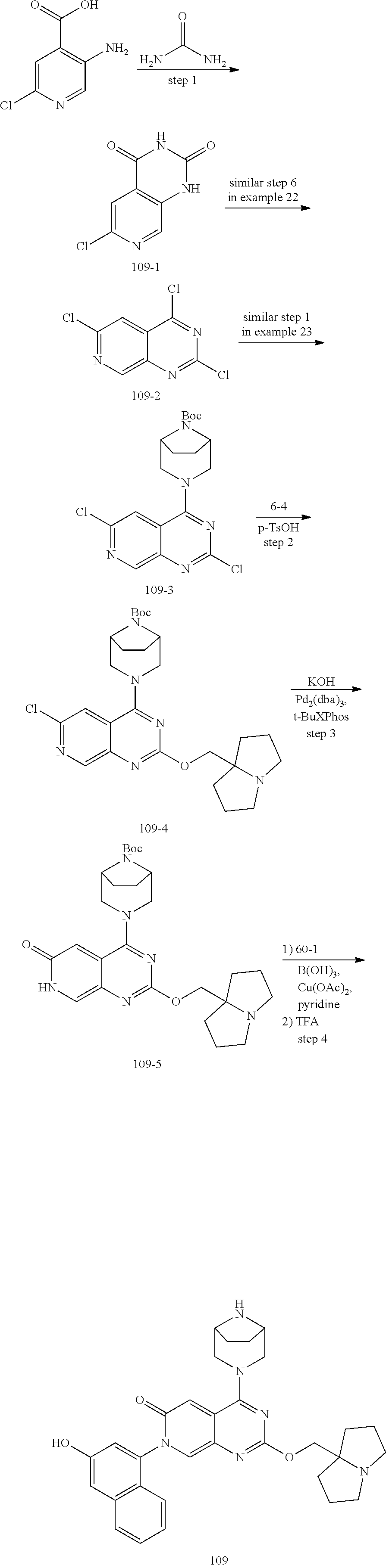

Step 1: A mixture of 5-amino-2-chloroisonicotinic acid (5.0 g, 29.0 mmol) and urea (17.5 g, 290 mmol) was stirred at 170° C. for 2 hours. After being cooled to room temperature, water (150 mL) was added and the mixture was refluxed under vigorous stirring for 20 min. The mixture was cooled and the precipitate formed was collected, washed with water and dried to afford 109-1 which was used directly in the next step without purification.

Compound 109-2 was prepared from 109-1 following the procedure for the synthesis of compound 67-7 in example 22.

Compound 109-3 was prepared from 109-2 following the procedure for the synthesis of compound 69-1 in example 23.

Step 2: A mixture of 109-3 (3.1 g, 7.5 mmol), p-toluenesulfonic acid (1.7 g, 10 mmol) and 6-4 (4.2 g, 30 mmol) was refluxed for 2 hours under nitrogen atmosphere. Then cooled and concentrated. The residue was suspended in saturated aqueous NaHCO₃ solution and the mixture was stirred at room temperature for 0.5 hour. The precipitate was collected by filtration to afford 109-4 which was used directly in the next step without purification.

Step 3: A mixture of 109-4 (2.7 g, 5.25 mmol), Pd₂(dba)₃ (480 mg, 0.53 mmol), t-BuXPhos (0.23 g, 0.53 mmol) and KOH (0.29 g, 5.25 mmol) in 1, 4-dioxane (80 mL) was degassed under nitrogen gas three times and stirred vigorously at 105° C. for 4 hours. After being cooled to room temperature, the mixture was diluted with ethyl acetate, washed with water and brine. The organic layer was dried over sodium sulfate, filtered and concentrated. The residue was purified by column chromatography on silica gel (dichloromethane/methanol=20/1) to afford 109-5.

Step 4: To a solution of 109-5 (50 mg, 0.10 mmol) in MeCN (3 mL) were added 60-1 (60 mg, 0.20 mmol), boric acid (12 mg, 0.20 mmol), pyridine (32 mg, 0.40 mmol) and Cu(OAc)₂ (40 mg, 0.20 mmol). The reaction was stirred for 24 hours at 60° C. under oxygen atmosphere. After being cooled to room temperature, TFA (1 mL) was added and the mixture was stirred at room temperature for 1 hour. The mixture was concentrated. The residue was purified by prep-HPLC (acetonitrile with 0.05% of TFA in water: 5% to 95%) to afford 109 as a 2 eq of TFA salt. LCMS (ESI, m/z): [M+H]⁺=539.4; ¹H NMR (400 MHz, methanol-d₄, ppm): δ 8.77 (s, 1H), 7.85 (d, J=8.4 Hz, 1H), 7.71 (d, J=8 Hz, 1H), 7.42-7.41 (m, 1H), 7.29 (s, 1H), 7.24-7.22 (m, 1H), 7.04 (d, J=2 Hz, 1H), 6.77 (d, J=2 Hz, 1H), 4.61 (s, 2H), 4.46 (d, J=13.6 Hz, 2H), 4.12 (s, 2H), 3.67-3.64 (m, 4H), 3.32-3.31 (m, 2H), 2.33-1.96 (m, 12H).

### Example 37 Synthesis of Compound 119

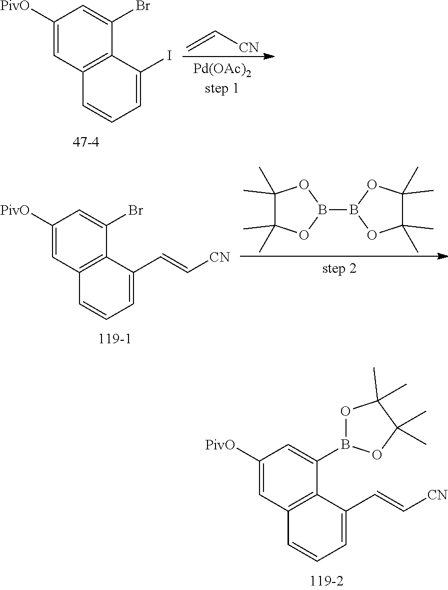

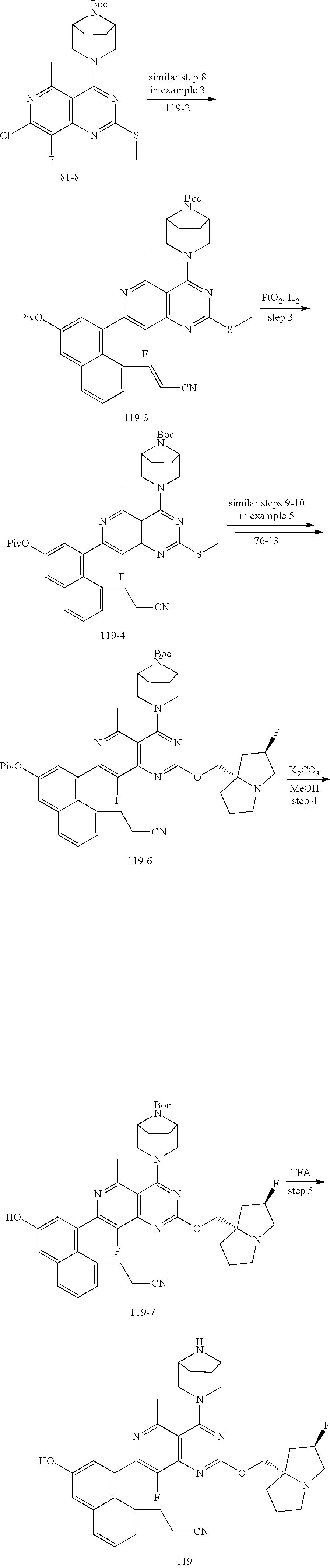

Step 1: To a solution of 47-4 (3.2 g, 7.4 mmol) in dimethylformamide (80 mL) were added acrylonitrile (784 mg, 14.8 mmol), potassium carbonate (410 mg, 3 mmol), triphenylphosphine (388 mg, 1.48 mmol) and palladium acetate (166 mg, 0.74 mmol). The reaction was stirred at 120° C. for 2 hours under nitrogen. The mixture was extracted with ethyl acetate and washed with brine. The organic layer was concentrated and the residue was purified by column chromatography on silica gel (petroleum ether to petroleum ether/ethyl acetate=10/1) to afford 119-1.

Step 2: To a solution of 119-1 (1.0 g, 2.8 mmol) in dioxane (100 mL) were added bis(pinacolato)diboron (1.4 g, 5.6 mmol), potassium acetate (824 mg, 8.4 mmol) and [1,1′-bis(diphenylphosphino)ferrocene]dichloropalladium(II) (205 mg, 0.28 mmol). The reaction was stirred at 85° C. for 3 hours under nitrogen. The mixture was extracted with ethyl acetate and washed with water. The organic layer was concentrated and the residue was purified by column chromatography on silica gel (petroleum ether to petroleum ether/ethyl acetate=10/1) to afford 119-2.

Compound 119-3 was prepared from 81-8 and 119-2 following the coupling procedure for the synthesis of compound 10 in example 3.

Step 3: To a solution of 119-3 (30 mg, 0.04 mmol) in ethanol (10 mL) was added platinum(IV) oxide (30 mg). The reaction was stirred at room temperature for 48 hours under hydrogen. The mixture was filtered and concentrated. The residue was purified by column chromatography on silica gel (petroleum ether to petroleum ether/ethyl acetate=1/1) to afford 119-4.

Compound 119-6 was prepared from 119-4 following the procedure for the synthesis of compound 60-10 in example 5.

Step 4: To a solution of 119-6 (5 mg, 0.006 mmol) in methanol (1.5 mL) was added potassium carbonate (3 mg, 0.018 mmol). The reaction was stirred for 1 hour at room temperature. The mixture was extracted with dichloromethane and washed with water. The organic layer was concentrated to afford 119-7 which was used directly in the next step without purification.

Step 5: A solution of 119-6 (3 mg, 0.004 mmol) and trifluoroacetic acid (1 mL) in dichloromethane (3 mL) was stirred at room temperature for 1 hour. The mixture was extracted with dichloromethane and washed with saturated aqueous sodium bicarbonate. The organic layer was concentrated and the residue was purified by prep-HPLC (acetonitrile with 0.05% of TFA in water: 5% to 30%) to afford 119 as a 3 eq of TFA salt. LCMS (ESI, m/z): [M+H]⁺=626.3; ¹H NMR (400 MHz, methanol-d₄, ppm): δ 7.71 (d, J=8.0 Hz, 1H), 7.39-7.35 (m, 1H), 7.32 (d, J=2.8 Hz, 1H), 7.17 (d, J=7.2 Hz, 1H), 7.08 (d, J=2.0 Hz, 1H), 5.62-5.50 (m, 1H), 4.97-4.90 (m, 4H), 4.72-4.69 (m, 3H), 4.30-3.50 (m, 6H) 3.48-3.43 (m, 1H), 2.85-2.63 (m, 5H), 2.59-2.50 (m, 2H), 2.48-2.31 (m, 4H), 2.15-2.00 (m, 4H).

### Example 38 Synthesis of Compound 116

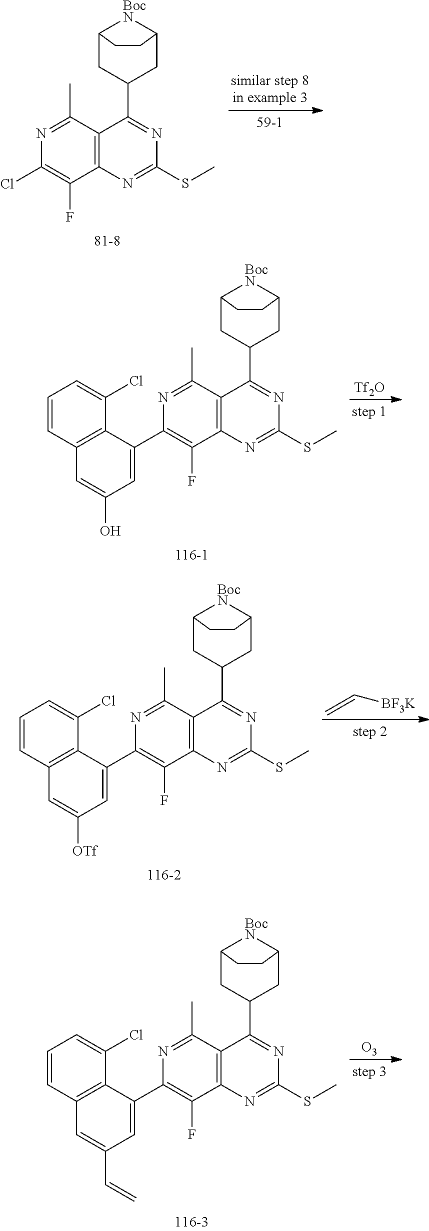

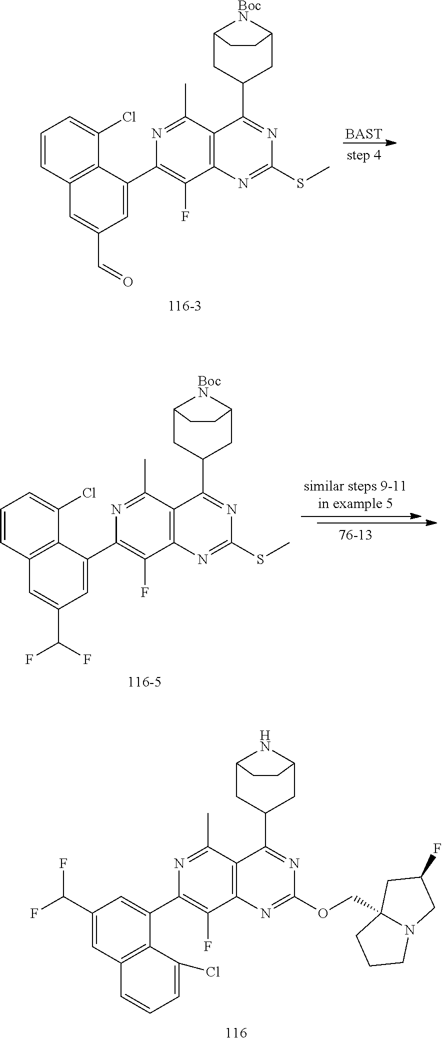

Compound 116-1 was prepared from 81-8 and 59-1 following the coupling procedure for the synthesis of compound 10 in example 3.

Step 1: To a mixture of 116-1 (595 mg, 1.0 mmol) and N, N-diisopropylethylamine (323 mg, 2.5 mmol) in dichloromethane (20 mL) was added trifluoromethanesulfonic anhydride (367 mg, 1.3 mmol) dropwise at 0° C. The mixture was stirred at 0° C. for 30 min. The reaction reaction was quenched with water. The mixture was extracted with dichloromethane. The combined organic layers were washed with brine, dried over sodium sulfate, filtered and concentrated. The residue was purified by column chromatography on silica gel (petroleum ether to petroleum ether/ethyl acetate=5/1) to afford 116-2.

Step 2: To a mixture of 116-2 (600 mg, 0.83 mmol) in isopropyl alcohol (12 mL) was added [1,1′-bis(diphenylphosphino)ferrocene]-dichloropalladium (II) (90 mg, 0.12 mmol), potassium vinyltrifluoroborate (166 mg, 1.24 mmol) and triethylamine (250 mg, 2.47 mmol) at room temperature under nitrogen atmosphere. The mixture was stirred at 80° C. for 3 hours. The mixture was diluted with water and extracted with ethyl acetate. The combined organic layers were washed with brine, dried over sodium sulfate, filtered and concentrated. The residue was purified by column chromatography on silica gel (petroleum ether to petroleum ether/ethyl acetate=5/1) to afford 116-3.

Step 3: A solution of 116-3 (430 mg, 0.71 mmol) in chloroform/methanol (6 mL/6 mL) was bubbled with ozone at −78° C. for 5 min. The reaction mixture was bubbled with nitrogen at the same temperature for 5 min, followed by addition of dimethylsulfide (0.5 mL). The mixture was stirred at room temperature for 16 hours and concentrated. The residue was purified by column chromatography on silica gel (petroleum ether to petroleum ether/ethyl acetate=2/1) to afford 116-4.

Step 4: To a mixture of 116-4 (260 mg, 0.43 mmol) in dichloromethane (8 mL) was added bis(2-methoxyethyl) aminosulfur trifluoride (947 mg, 4.3 mmol) at 0° C. The mixture was stirred at room temperature for 16 hours. The reaction was quenched with saturated aqueous sodium bicarbonate and extracted with dichloromethane. The combined organic layers were washed with brine, dried over sodium sulfate, filtered and concentrated. The residue was purified by column chromatography on silica gel (petroleum ether to petroleum ether/ethyl acetate=4/1) to afford 116-5.

Compound 116 was prepared from 116-5 following the procedure for the synthesis of compound 60 in example 5 as a 3 eq of TFA salt. LCMS (ESI, m/z): [M+H]⁺=641.3; ¹H NMR (400 MHz, methanol-d₄, ppm): δ 8.32 (d, J=1.2 Hz, 1H), 8.11 (d, J=7.6 Hz, 1H), 7.77-7.67 (m, 2H), 7.65-7.55 (m, 1H), 7.01 (t, J=56.0 Hz, 1H), 5.68-5.46 (m, 1H), 4.76-4.33 (m, 4H), 4.24-4.09 (m, 2H), 4.08-3.77 (m, 5H), 3.49-3.27 (m, 1H), 2.89-2.50 (m, 5H), 2.48-2.29 (m, 3H), 2.24-1.68 (m, 5H). ¹⁹F NMR (376 MHz, methanol-d₄, ppm): δ −112.6 (2F), −145.2 (1F), −174.1 (1F).

### Example 39 Synthesis of Compound 122

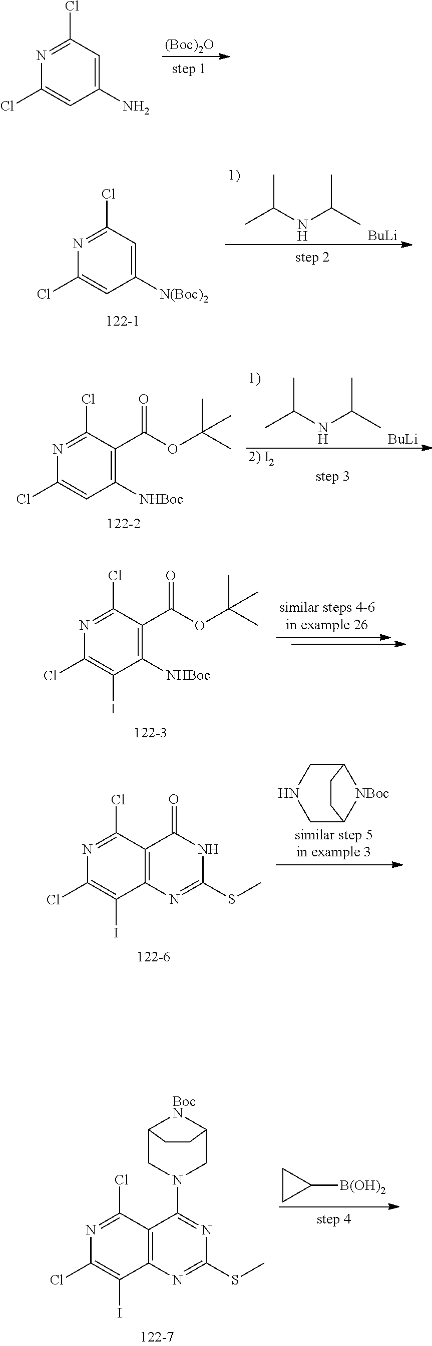

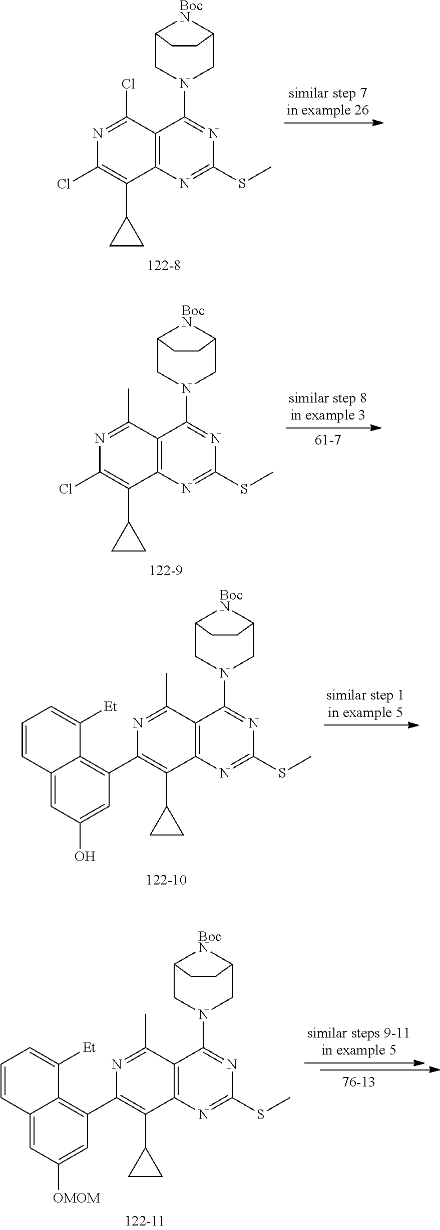

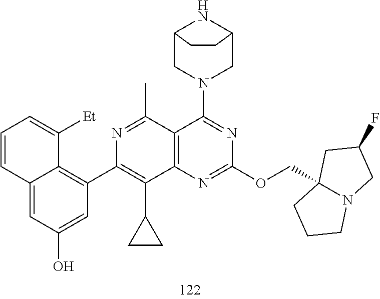 [SMILES: CCc1cccc2cc(O)cc(-c3nc(C)c4c(N5CC6CCC(C5)N6)nc(OC[C@@]56CCCN5C[C@H](F)C6)nc4c3C3CC3)c12]

Step 1: To a mixture of 2,6-dichloropyridin-4-amine (100 g, 0.62 mol) in heptane (150 mL) were added di-tert-butyl dicarbonate (378 g, 1.73 mol) and 4-dimethylaminopyridine (7.6 g, 62 mmol) at room temperature. And then the reaction mixture was stirred overnight at 40° C. The reaction mixture was concentrated and the residue was slurried in petroleum ether. Then, the mixture was filtered and dried to afford 122-1.

Step 2: To a solution of diisopropylamine (38.7 g, 300 mmol) in tetrahydrofuran (250 mL) was added n-butyllithium (120 mL, 300 mmol) dropwised at −78° C. under N₂ atmosphere. The mixture was stirred −78° C. for 1 hour. To above mixture was added a solution of 122-1 (83 g, 230 mmol) in tetrahydrofuran (400 mL) dropwised at −78° C. under N₂ atmosphere. The resulting mixture was stirred at −78° C. for 1 hour. The reaction was quenched with acetic acid and the mixture was diluted with ethyl acetate, washed with water and brine. The organic layer was dried over sodium sulfate, filtered and concentrated. The residue was slurried in heptane. Then, the mixture was filtered and dried to afford 122-2.

Step 3: To a solution of diisopropylamine (5.05 g, 50 mmol) in tetrahydrofuran (20 mL) was added n-butyllithium (20 mL, 50 mmol, 2.5 M in tetrahydrofuran) at −78° C. The mixture was then stirred at −78° C. for 0.5 hour. To above mixture was added a solution of 122-2 (7.3 g, 20 mmol) in tetrahydrofuran (10 mL) dropwise at −78° C. The resulting mixture was stirred at −78° C. for 1 hour. To above mixture was added a solution of iodine (6.1 g, 24 mmol) in tetrahydrofuran (10 mL) dropwise. The mixture was then stirred at −78° C. for 0.5 hour. The reaction was quenched with acetic acid. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=10/1) to afford 122-3.

Compound 122-6 was prepared from 122-3 following the procedure for the synthesis of compound 81-6 in example 26.

Compound 122-7 was prepared from 122-6 following the procedure for the synthesis of compound 10-5 in example 3.

Step 4: A mixture of 122-7 (2 g, 3.4 mmol), cyclopropylboronic acid (610 mg, 4.1 mmol), palladium(II) acetate (77 mg, 0.34 mmol), 2-dicyclohexylphosphino-2′,6′-dimethoxybiphenyl (282 mg, 0.68 mmol) and tripotassium phosphate (2.2 g, 10.3 mmol) in toluene/water (10/1, 22 mL) was stirred at 95° C. for 3 hours under N₂ atmosphere. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=9/1) to afford 122-8.

Compound 122-9 was prepared from 122-8 following the procedure for the synthesis of compound 81-8 in example 26.

Compound 122-10 was prepared from 59-1 and 122-9 following the coupling procedure for the synthesis of compound 10 in example 3.

Compound 122-11 was prepared from 122-10 following the procedure for the synthesis of compound 60-1 in example 5.

Compound 122 was prepared from 122-11 following the procedure for the synthesis of compound 60 in example 5. LCMS (ESI, m/z): [M+H]⁺=623.4; ¹H NMR (400 MHz, methanol-d₄, ppm): δ 7.65 (d, J=8.4 Hz, 1H), 7.39 (t, J=7.6 Hz, 1H), 7.32 (d, J=2.4 Hz, 1H), 7.22 (d, J=7.2 Hz, 1H), 7.06 (d, J=2.4 Hz, 1H), 5.66-5.51 (m, 1H), 4.79-4.64 (m, 2H), 4.62-4.37 (m, 1H), 4.23-4.11 (m, 2H), 4.07-3.81 (m, 5H), 3.52-3.42 (m, 1H), 3.35-3.30 (m, 1H), 2.73 (s, 3H), 2.67-2.52 (m, 2H), 2.49-2.28 (m, 5H), 2.26-1.68 (m, 6H), 1.28-1.19 (m, 1H), 1.18-1.07 (m, 1H), 0.98 (t, J=7.2 Hz, 3H), 0.77-0.59 (m, 2H).

### Example 40 Synthesis of Compound 128

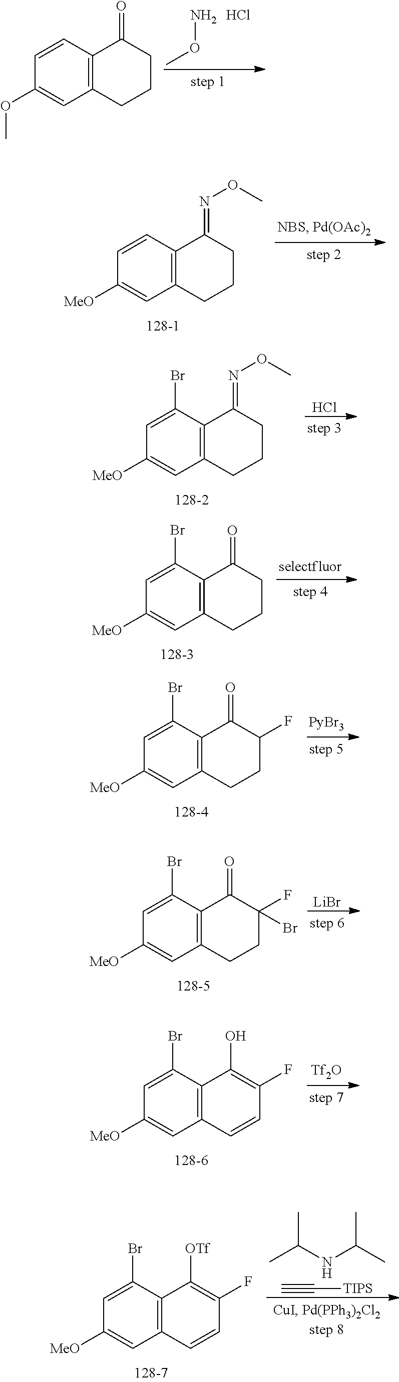

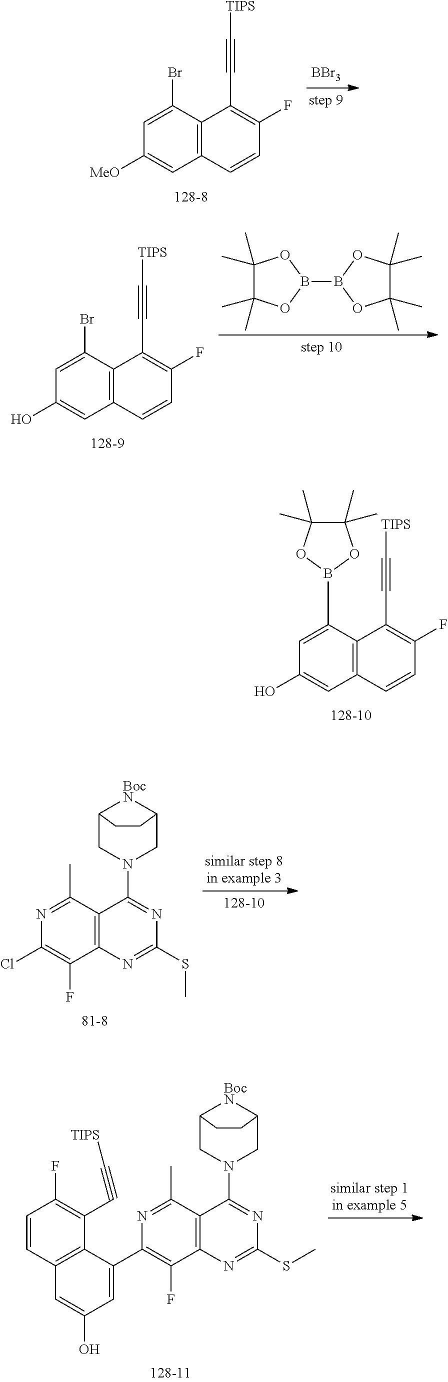

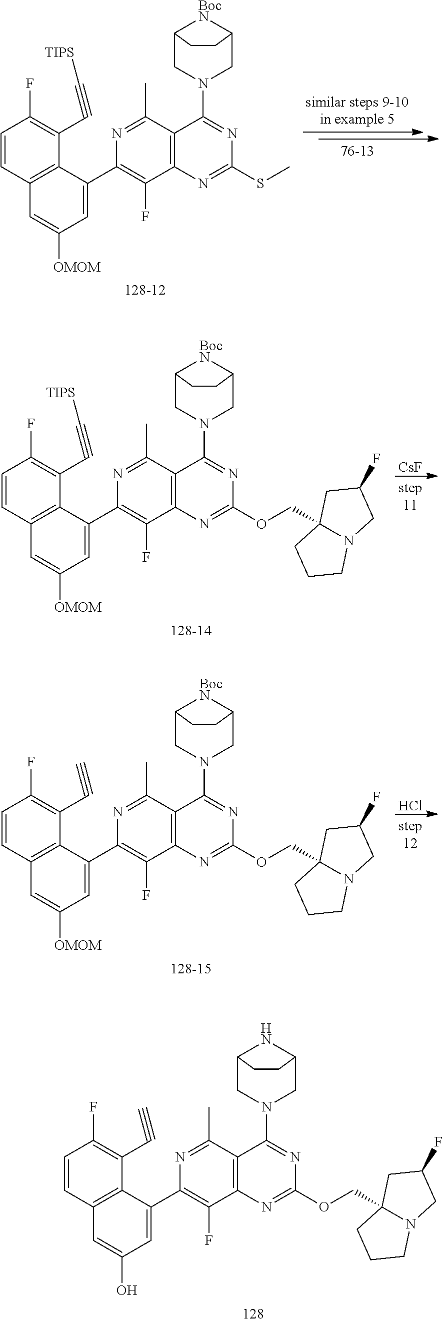

Step 1: A mixture of 6-methoxy-3,4-dihydronaphthalen-1 (2H)-one (50 g, 280 mmol), O-methylhydroxylamine hydrochloride (28 g, 336 mmol) in ethanol (500 mL) and pyridine (33 g, 420 mmol) was stirred at room temperature for 2 hours. The mixture was concentrated to give an oil. The oil was dissolved in dichloromethane, washed with 2N hydrochloric acid, saturated aqueous sodium bicarbonate, brine, dried over sodium sulfate, filtered and concentrated to afford 128-1 which was used directly in the next step without purification.

Step 2: A mixture of 128-1 (25 g, 120 mmol), palladium (II) acetate (1.3 g, 6 mmol), N-bromosuccinimide (21 g, 120 mmol) in acetic acid (400 mL) was stirred at 80° C. for 1 hour. The solution was poured into water and filtered. The filter cake was dried to afford 128-2 which was used directly in the next step without purification.

Step 3: A suspension of 128-2 (18 g, 80 mmol) in concentrated hydrochloric acid (100 mL) and dioxane (150 mL) was stirred at reflux for 1 hour. The mixture was concentrated, and the residue was dissolved in ethyl acetate, washed with 1 N NaOH, water, brine, and concentrated to afford the crude product. The product was purified by column chromatography on silica gel (petroleum ether to petroleum ether/ethyl acetate=4/1) to afford 128-3.

Step 4: To a mixture of 1-chloromethyl-4-fluoro-1,4-diazoniabicyclo[2.2.2]octane bis(tetrafluoroborate) (8.14 g, 23 mmol) and 128-3 (5.1 g, 20 mmol) in methanol (80 mL) was added concentrated sulfuric acid (0.1 mL). The mixture was stirred at 50° C. for 5 hours under N₂ atmosphere. The mixture was concentrated, diluted with ethyl acetate, washed with water and brine. The organic layer was dried over sodium sulfate, filtered and concentrated. The residue was triturated with petroleum ether/ethyl acetate (10/1) to afford 128-4.

Step 5: The mixture of 128-4 (4.63 g, 16.96 mmol) and pyridinium tribromide (5.97 g, 18.66 mmol) in acetonitrile (46 mL) was stirred at 60° C. for 30 min under N₂ atmosphere. The mixture was diluted with ethyl acetate, washed with water and brine. The organic layer was dried over sodium sulfate, filtered and concentrated. The residue was triturated with petroleum ether/ethyl acetate (10/1) to afford 128-5.

Step 6: A mixture of 128-5 (5.4 g, 15.38 mmol), lithium bromide (2.94 g, 33.85 mmol) in N, N-dimethylformamide (15 mL) was stirred at 100° C. for 30 min under N₂ atmosphere. After being cooled to room temperature, the mixture was diluted with ethyl acetate, washed with water and brine. The organic layer was dried over sodium sulfate, filtered and concentrated. The residue was triturated with petroleum ether/ethyl acetate (10/1) to afford 128-6.

Step 7: To a mixture of 128-6 (12.96 g, 48 mmol) and pyridine (11.4 g, 144 mmol) in dichloromethane (150 mL) was added triflic anhydride (16.2 g, 57.6 mmol) dropwise at 0° C. under N₂ atmosphere. The mixture was stirred at room temperature for 1 hour. The reaction mixture was washed with water and brine, dried over sodium sulfate, filtered and concentrated. The residue was purified by column chromatography on silica gel (petroleum ether to petroleum ether/ethyl acetate=8/1) to afford 128-7.

Step 8: To a mixture of 128-7 (18 g, 45 mmol) in N,N-dimethylformamide (300 mL) were added triisopropylsilylacetylene (12.3 g, 67.5 mmol), diisopropylamine (45.5 g, 450 mmol), CuI (855 mg, 4.5 mmol) and bis(triphenylphosphine)palladium(II) chloride (1.58 g, 2.25 mmol) under N₂ atmosphere. The mixture was stirred at 50° C. for 16 hours. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=10/1) to afford 128-8.

Step 9: To a mixture of 128-8 (10.6 g, 24.4 mmol) in dichloromethane (150 mL) was added boron tribromide (14.6 mL, 29.2 mmol, 2 M in dichloromethane) dropwise at −78° C. under N₂ atmosphere. The mixture was stirred at 0° C. for 3 hours. The reaction was quenched with ice-water. The organic layer was washed with saturated aqueous sodium hydrogencarbonate and brine, dried over sodium sulfate, filtered and concentrated. The residue was purified by column chromatography on silica gel (petroleum ether to petroleum ether/ethyl acetate=6/1) to afford 128-9.

Step 10: A mixture of 128-9 (8.89 g, 19 mmol), bis(pinacolato)diboron (9.65 g, 38 mmol), potassium acetate (5.59 g, 57 mmol), tris(dibenzylideneacetone)dipalladium (870 mg, 0.95 mmol) and tricyclohexyl phosphine (532 mg, 1.9 mmol) in dioxane (100 mL) was stirred at 105° C. for 10 hours under N₂ atmosphere. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=8/1) to afford 128-10.

Compound 128-11 was prepared from 81-8 and 128-10 following the coupling procedure for the synthesis of compound 10 in example 3.

Compound 128-12 was prepared from 128-11 following the procedure for the synthesis of compound 60-1 in example 5.

Compound 128-14 was prepared from 128-12 following the procedure for the synthesis of compound 60-10 in example 5.

Step 11: A mixture of 128-14 (100 mg, 0.11 mmol) and caesium fluoride (166 mg, 1.1 mmol) in N,N-dimethylformamide (5.5 mL) was stirred at 50° C. for 30 min under N₂ atmosphere. The mixture was cooled, diluted with ethyl acetate, washed with water and brine. The organic layer was dried over sodium sulfate, filtered and concentrated. The residue was purified by prep-HPLC (acetonitrile with 0.05% of TFA in water: 15% to 95%) to afford 128-15.

Step 12: A mixture of 128-15 (50 mg, 0.069 mol) and 0.7M hydrochloric acid solution in ethyl acetate (2 mL) was stirred at 50° C. for 2 hours under N₂ atmosphere. The mixture was concentrated. The residue was purified by prep-HPLC (acetonitrile with 0.05% of TFA in water: 5% to 95%) to afford 128 as a 3 eq of TFA salt. LCMS (ESI, m/z): [M+H]⁺=615.3; ¹H NMR (400 MHz, methanol-d₄, ppm): δ 7.90-7.86 (m, 1H), 7.37-7.32 (m, 2H), 7.21 (s, 1H), 5.65-5.51 (m, 1H), 4.78-4.66 (m, 2H), 4.65-4.41 (m, 2H), 4.28-4.10 (m, 2H), 4.08-3.83 (m, 5H), 3.52-3.43 (m, 1H), 3.30-3.27 (m, 1H), 2.86-2.52 (m, 5H), 2.50-2.29 (m, 3H), 2.26-1.72 (m, 5H). ¹⁹F NMR (376 MHz, methanol-d₄, ppm): δ −111.55 (1F), −144.45 (1F), −174.15 (1F).

### Example 41 Synthesis of Compound 127

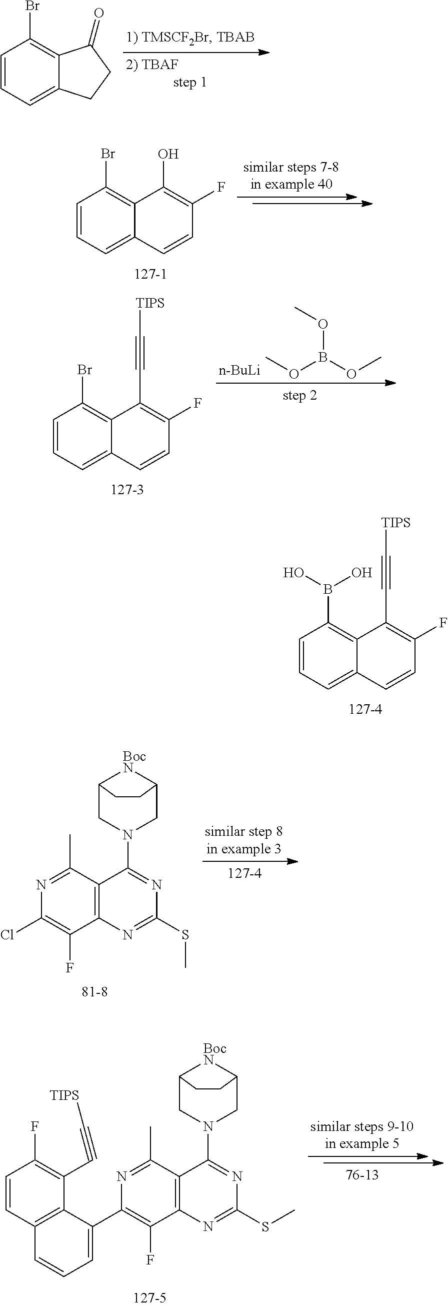

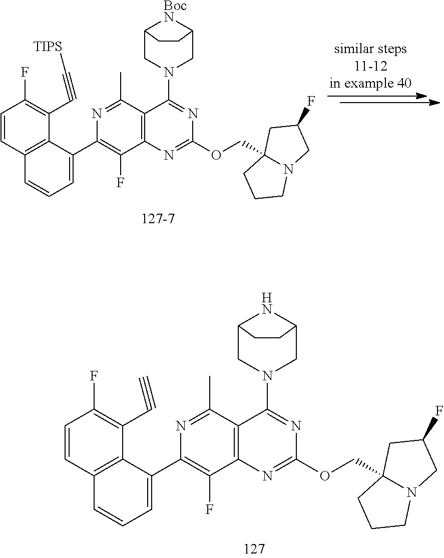

Step 1: To a mixture of 7-bromo-1-indanone (9.45 g, 45 mmol), tetrabutylammonium bromide (1.45 g, 4.5 mmol) in toluene (100 mL) was added (bromodifluoromethyl)trimethylsilane (27.3 g, 135 mmol). The mixture was stirred at 110° C. for 16 hours under N₂ atmosphere. After being cooled to room temperature, tetrabutylammonium fluoride (9 mL, 9 mmol, 1 M in tetrahydrofuran) was added and the mixture was stirred for 1 hour. The reaction mixture was washed with water and brine, dried over sodium sulfate, filtered and concentrated. The residue was purified by column chromatography on silica gel (petroleum ether to petroleum ether/ethyl acetate=10/1) to afford 127-1.

Compound 127-3 was prepared from 127-1 following the procedure for the synthesis of compound 128-8 in example 40.

Step 2: To a solution of 127-1 (870 mg, 2.15 mmol) in tetrahydrofuran (10 mL) was added dropwise n-butyllithium (1.29 mL, 3.22 mmol) at −78° C. over 10 min under N₂ atmosphere. The mixture was stirred for 30 min and trimethyl borate (671 mg, 6.45 mmol) was added thereto. The resulting mixture was allowed to warm to room temperature and stirred for another 2 hours. The reaction was quenched with 2M hydrochloric acid and extracted with ethyl acetate. The combined organic layers were washed with brine, dried over sodium sulfate, filtered and concentrated. The residue was purified by column chromatography on silica gel (dichloromethane to dichloromethane/methanol=10/1) to afford 127-4.

Compound 127-5 was prepared from 81-8 and 127-4 following the coupling procedure for the synthesis of compound 10 in example 3.

Compound 127-7 was prepared from 127-5 following the procedure for the synthesis of compound 60-10 in example 5.

Compound 127 was prepared from 127-7 following the procedure for the synthesis of compound 128 in example 40 as a 3 eq of TFA salt. LCMS (ESI, m/z): [M+H]⁺=623.4; ¹H NMR (400 MHz, methanol-d₄, ppm): δ 8.15-8.11 (m, 2H), 7.69-7.62 (m, 2H), 7.46 (t, J=8.8 Hz, 1H), 5.65-5.51 (m, 1H), 4.72-4.70 (m, 2H), 4.68-4.33 (m, 2H), 4.27-4.13 (m, 2H), 4.09-3.82 (m, 5H), 3.50-3.43 (m, 1H), 3.37-3.36 (m, 1H), 2.71-2.55 (m, 5H), 2.47-2.31 (m, 3H), 2.24-1.75 (m, 5H). ¹⁹F NMR (376 MHz, methanol-d₄, ppm): δ −106.67 (1F), −144.68 (1F), −174.15 (1F).

### Example 42 Synthesis of Compound 123

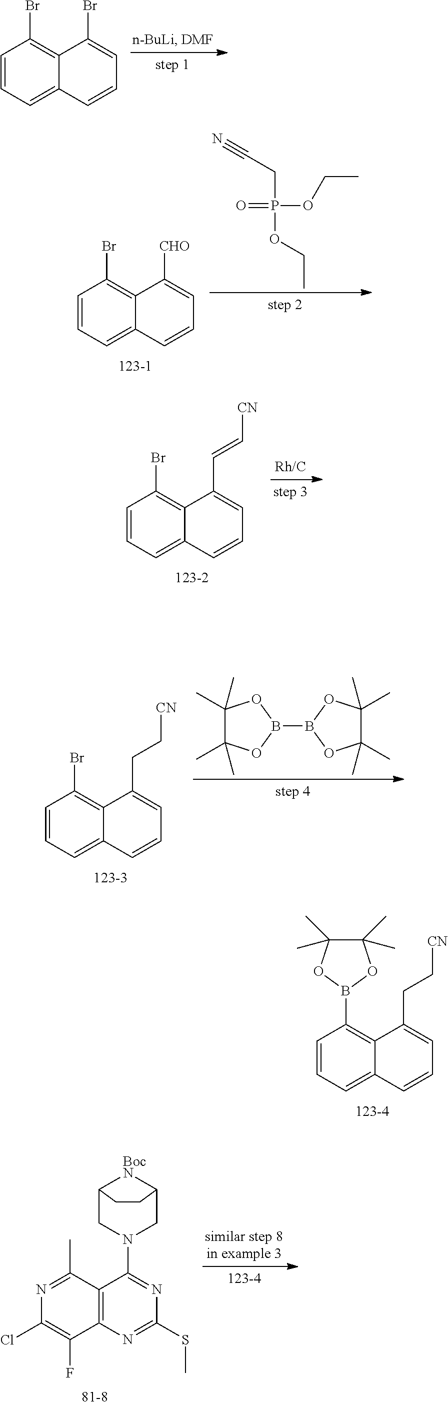

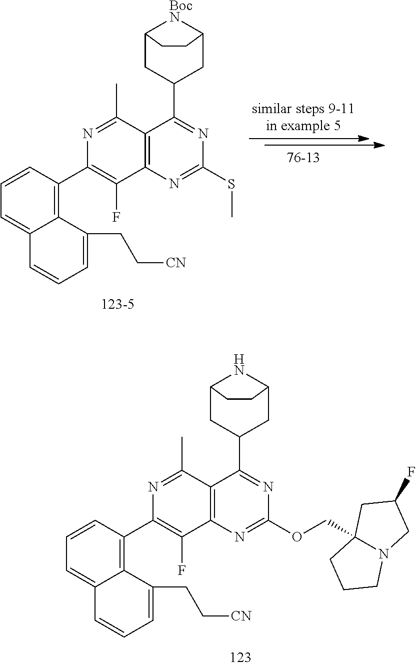 [SMILES: CSc1nc(C2CC3CCC(C2)N3C(=O)OC(C)(C)C)c2c(C)nc(-c3cccc4cccc(CCC#N)c34)c(F)c2n1 ; Cc1nc(-c2cccc3cccc(CCC#N)c23)c(F)c2nc(OC[C@@]34CCCN3C[C@H](F)C4)nc(C3CC4CCC(C3)N4)c12]

Step 1: BuLi (15 mL, 37.53 mmol) was added dropwise to the solution of 1,8-dibromonaphthalene (7.2 g, 25 mmol) in THF (100 mL) at −70° C. under N₂ atmosphere. The resulting mixture was stirred at −70° C. for 30 min. Then DMF (3.5 mL) was added at −70° C. under N₂ atmosphere. The mixture was allowed to warm to room temperature. After 1 hour, the reaction was quenched with sat. aq. NH₄Cl solution, diluted with water and extracted with ethyl acetate. The combined organic layers were washed with brine, dried over sodium sulfate, filtered and concentrated. The residue was purified by silica gel column chromatography (petroleum ether/ethyl acetate=10/1) to afford 123-1.

Step 2: Diethyl(cyanomethyl)phosphonate (4.29 g, 24.3 mmol) was added dropwise to a suspension of NaH (1.94 g, 48.5 mmol) in THF (57 mL) at 0° C. under N₂ atmosphere. The resulting mixture was stirred at 0° C. for 1 hour. A solution of 123-1 (1.9 g, 8.09 mmol) in THE (23 mL) was added at 0° C. under N₂ atmosphere. The mixture was allowed to warm to room temperature. After 1 hour, the reaction was quenched with sat. aq. NH₄Cl solution, diluted with water and extracted with ethyl acetate. The combined organic layers were washed with brine, dried over sodium sulfate, filtered and concentrated. The residue was purified by silica gel column chromatography (petroleum ether/ethyl acetate=10/1) to afford 123-2.

Step 3: Rh/C (374 mg, 5%) was added to a solution of 123-2 (1.87 g, 7.25 mmol) in EtOH (73 mL) at room temperature. The resulting mixture was stirred at room temperature for 18 hours under H₂ atmosphere. The catalyst was filtered off, and the filtrate was concentrated. The residue was purified by silica gel column chromatography (petroleum ether/ethyl acetate=10/1) to afford 123-3.

Step 4: A mixture of 123-3 (819 mg, 3.15 mmol), 4,4,4′,4′,5,5,5′,5′-octamethyl-2,2′-bi(1,3,2-dioxaborolane) (1.6 g, 6.3 mmol), KOAc (926 mg, 9.45 mmol) and Pd(dppf)Cl₂ (231 mg, 0.315 mmol) in dioxane (15 mL) was stirred at 95° C. for 18 hours under N₂ atmosphere. The mixture was diluted with water and extracted with ethyl acetate. The combined organic layers were washed with brine, dried over sodium sulfate, filtered and concentrated. The residue was purified by silica gel column chromatography (petroleum ether/ethyl acetate=10/1) to afford 123-4.

Compound 123-5 was prepared from 81-8 and 123-4 following the coupling procedure for the synthesis of compound 10 in example 3.

Compound 123 was prepared from 123-5 following the procedure for the synthesis of compound 60 in example 5. LCMS (ESI, m/z): [M+H]⁺=610.4; ¹H NMR (400 MHz, methanol-d₄, ppm): δ 8.10-8.08 (dd, J=8.0, 1.2 Hz, 1H), 7.96 (d, J=7.2 Hz, 1H), 7.62 (t, J=8.0 Hz, 1H), 7.53-7.48 (m, 2H), 7.42 (d, J=6.4 Hz, 1H), 5.63-5.50 (m, 1H), 5.01-4.98 (m, 1H), 4.71-4.66 (m, 2H), 4.22-3.79 (m, 8H), 3.49-3.42 (m, 1H), 2.75-2.30 (m, 12H), 2.17-2.01 (m, 4H), 1.77-1.70 (m, 1H).

### Example 43 Synthesis of Compound 124

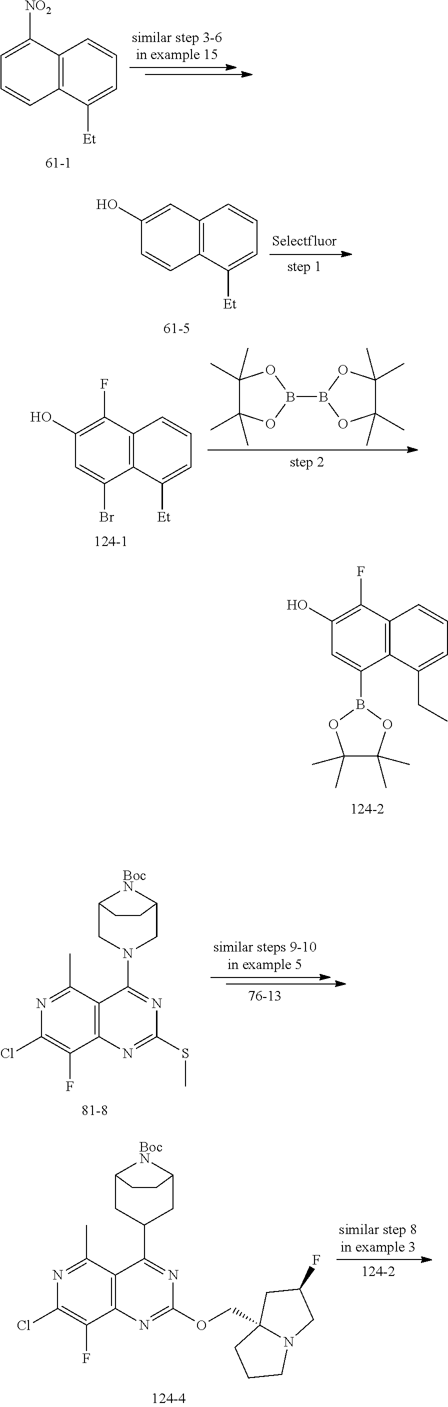

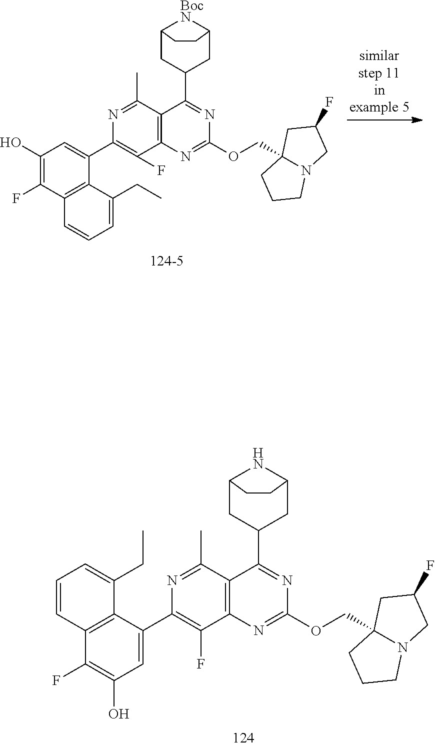 [SMILES: CCc1cccc2c(F)c(O)cc(-c3nc(C)c4c(C5CC6CCC(C5)N6C(=O)OC(C)(C)C)nc(OC[C@@]56CCCN5C[C@H](F)C6)nc4c3F)c12 ; CCc1cccc2c(F)c(O)cc(-c3nc(C)c4c(C5CC6CCC(C5)N6)nc(OC[C@@]56CCCN5C[C@H](F)C6)nc4c3F)c12]

Compound 61-5 was prepared from 61-1 following the procedure for the synthesis of compound 75-6 in example 15.

Step 1: To a solution of 61-5 (20 mg, 0.08 mmol) in acetonitrile (0.5 mL) was added selectfluor (31.04 mg, 0.088 mmol). This reaction mixture was stirred at 85° C. for 20 hours. This reaction mixture was concentrated. The residue was purified by prep-TLC (petroleum ether/ethyl acetate=10/1) to afford 124-1.

Step 2: To a solution of 124-1 (200 mg, 0.74 mmol) and 4,4,5,5-tetramethyl-2-(4,4,5,5-tetramethyl-1,3,2-dioxaborolan-2-yl)-1,3,2-dioxaborolane (754.90 mg, 2.97 mmol) in dioxane (5 mL) was added KOAc (3.72 mmol) and Pd(dppf)Cl₂ (54.33 mg, 0.074 mmol). The reaction mixture was stirred at 100° C. for 12 hours. The mixture was filtered and concentrated. The residue was purified by prep-TLC (petroleum ether/ethyl acetate=10/1) to afford 124-2.

Compound 124-4 was prepared from 124-2 following the procedure for the synthesis of compound 60-10 in example 5.

Compound 124-5 was prepared from 124-2 and 124-4 following the coupling procedure for the synthesis of compound 10 in example 3.

Compound 124 was prepared from 124-5 following the procedure for the synthesis of compound 60 in example 5. LCMS (ESI, m/z): [M+H]⁺=619.3; ¹H NMR (400 MHz, methanol-d₄, ppm): δ 7.96 (d, J=8.4 Hz, 1H), 7.50 (t, J=7.2 Hz, 1H), 7.27 (d, J=6.8 Hz, 1H), 7.16-7.14 (dd, J=8.8, 1.2 Hz, 1H), 5.65-5.52 (m, 1H), 4.76-4.71 (m, 2H), 4.55-4.52 (m, 1H), 4.22-3.85 (m, 7H), 3.51-3.44 (m, 1H), 3.36-3.32 (m, 1H), 2.74-2.45 (m, 5H), 2.36-1.86 (m, 10H), 0.89 (t, J=7.2 Hz, 3H).

### Example 44 Synthesis of Compound 125

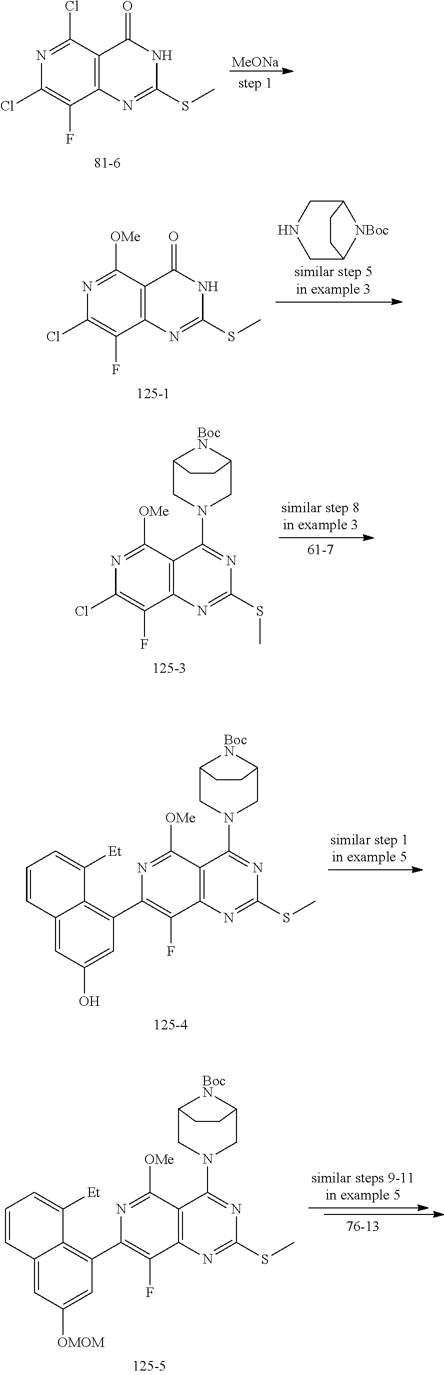

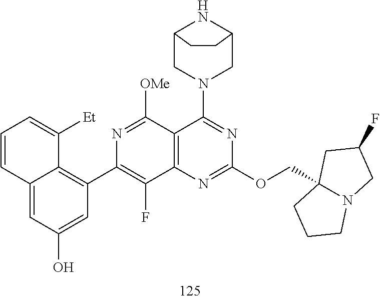 [SMILES: CCc1cccc2cc(O)cc(-c3nc(OC)c4c(N5CC6CCC(C5)N6)nc(OC[C@@]56CCCN5C[C@H](F)C6)nc4c3F)c12]

Step 1: To a mixture of 81-6 (11.16 g, 40 mmol) in methanol (10 mL) and dimethylacetamide (60 mL) was added sodium methoxide (10.8 g, 200 mmol). The mixture was stirred at 50° C. for 16 hours. The mixture was cooled, diluted with water and adjusted to around pH 3 with conc. HCl. Then, the mixture was filtered and washed with water. The filter cake was triturated in methanol and then filtered to afford 125-1.

Compound 125-3 was prepared from 125-1 following the procedure for the synthesis of compound 10-5 in example 3.

Compound 125-4 was prepared from 61-7 and 125-3 following the coupling procedure for the synthesis of compound 10 in example 3.

Compound 125-5 was prepared from 125-4 following the procedure for the synthesis of compound 60-1 in example 5.

Compound 125 was prepared from 125-5 following the procedure for the synthesis of compound 60 in example 5. LCMS (ESI, m/z): [M+H]⁺=617.3; ¹H NMR (400 MHz, methanol-d₄, ppm): δ 7.62 (d, J=8.0 Hz, 1H), 7.36 (t, J=7.2 Hz, 1H), 7.26 (d, J=2.4 Hz, 1H), 7.16 (d, J=7.2 Hz, 1H), 7.02-6.99 (m, 1H), 5.64-5.51 (m, 1H), 4.72-4.40 (m, 4H), 4.24-4.20 (m, 2H), 4.05-3.70 (m, 8H), 3.51-3.42 (m, 1H), 2.77-2.34 (m, 7H), 2.24-2.03 (m, 5H), 0.95-0.90 (m, 3H). ¹⁹F NMR (376 MHz, methanol-d₄, ppm): δ −149.89 (1F), −174.19 (1F).

### Example 45 Synthesis of Compound 139

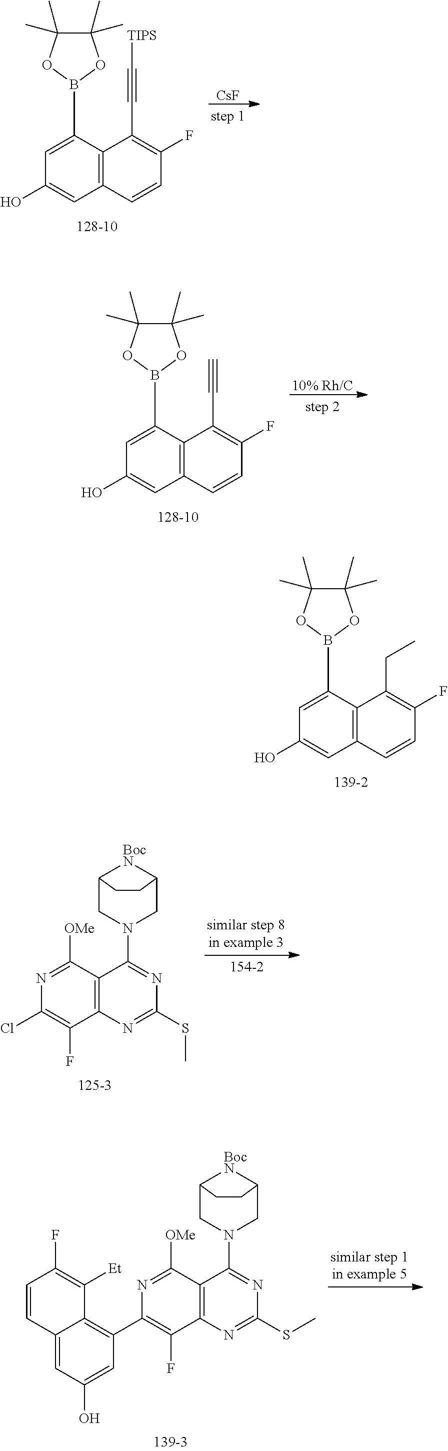

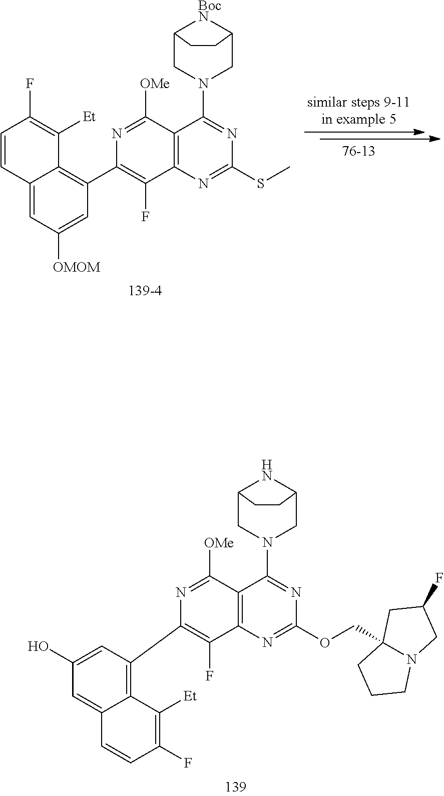

Step 1: A mixture of 128-10 (6.57 g, 14 mmol) and caesium fluoride (4.26 g, 28 mmol) in N, N-dimethylformamide (70 mL) was stirred at 50° C. for 30 min. The reaction mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=4/1) to afford 139-1.

Step 2: A mixture of 139-1 (3.59 g, 11.47 mmol), and 10% Rh/C (718 mg) in ethyl acetate (115 mL) was stirred at 45° C. for 5 hours. The mixture was filtered and concentrated. The residue was purified by column chromatography on silica gel (petroleum ether to petroleum ether/ethyl acetate=4/1) to afford 139-2.

Compound 139-3 was prepared from 139-2 and 139-3 following the coupling procedure for the synthesis of compound 10 in example 3.

Compound 139-4 was prepared from 139-3 following the procedure for the synthesis of compound 60-1 in example 5.

Compound 139 was prepared from 139-4 following the procedure for the synthesis of compound 60 in example 5. LCMS (ESI, m/z): [M+H]⁺=635.3; ¹H NMR (400 MHz, methanol-d₄, ppm): δ 7.69-7.64 (m, 1H), 7.29-7.22 (m, 2H), 7.04 (s, 1H), 5.64-5.51 (m, 1H), 4.72-4.64 (m, 2H), 4.52-4.45 (m, 2H), 4.24-4.19 (m, 2H), 4.06-3.88 (m, 6H), 3.78-3.73 (m, 2H), 3.52-3.41 (m, 1H), 2.79-2.54 (m, 3H), 2.51-2.30 (m, 4H), 2.25-2.01 (m, 5H), 0.85-0.82 (m, 3H). ¹⁹F NMR (376 MHz, methanol-d₄, ppm): δ −121.40 (1F), −149.53 (1F), −174.25 (1F).

### Example 46 Synthesis of Compound 136

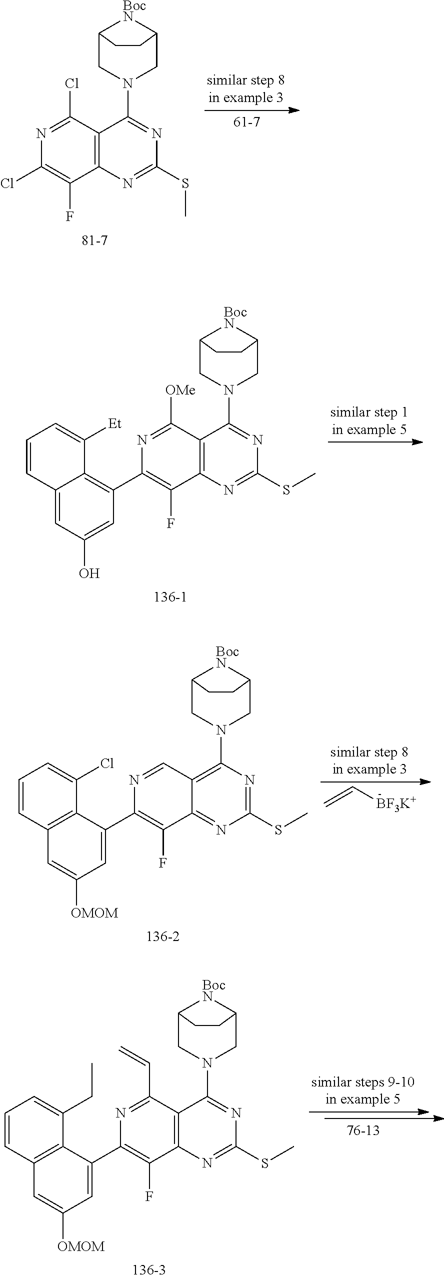

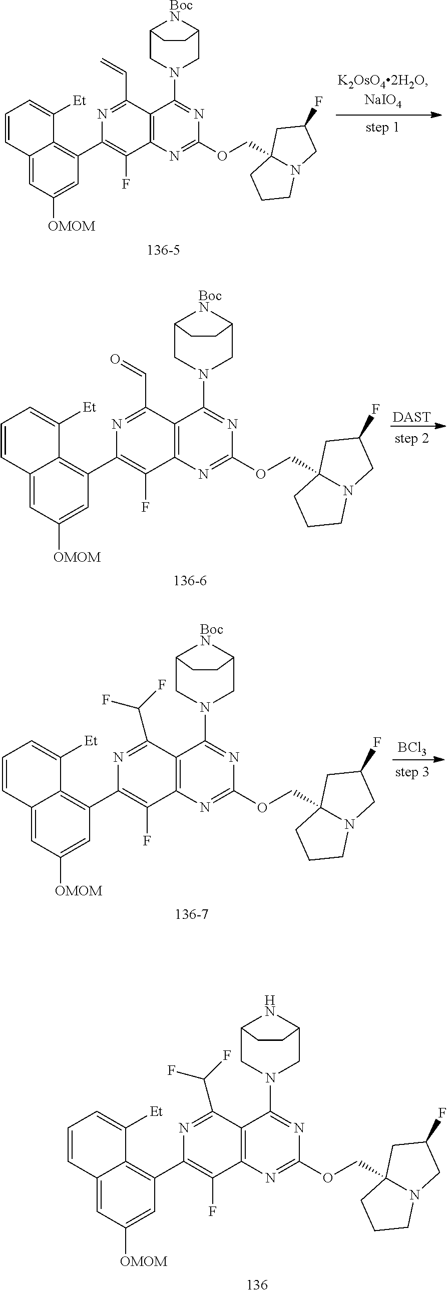

Compound 136-1 was prepared from 81-7 and 136-1 following the coupling procedure for the synthesis of compound 10 in example 3.

Compound 136-2 was prepared from 136-1 following the procedure for the synthesis of compound 60-1 in example 5.

Compound 136-3 was prepared from 136-2 following the coupling procedure for the synthesis of compound 10 in example 3.

Compound 136-5 was prepared from 136-3 following the procedure for the synthesis of compound 60-10 in example 5.

Step 1: To a solution of 136-5 (130 mg, 0.17 mmol) in THF (4 mL) were added a solution of potassium osmate (VI) dihydrate (1.9 mg, 0.0052 mmol) in water (2 mL) and a solution of NaIO₄ (55 mg, 0.26 mmol) in water (2 mL) dropwise at 0° C. The mixture was stirred at room temperature for 2 hours and water was added. The mixture was extracted with ethyl acetate. The combined organic layers were washed with brine, dried over sodium sulfate, filtered and concentrated. The residue was purified by prep-HPLC (0.05% of TFA in water with acetonitrile: 5% to 95%) to afford 136-6.

Step 2: To a solution of 136-6 (69 mg, 0.091 mmol) in dichloromethane (6 mL) was added DAST (265 mg, 1.65 mmol) dropwise and the resulting mixture was stirred at 25° C. for 32 hours. After being quenched with sat. aq. NaHCO₃ solution, the mixture was extracted with dichloromethane. The combined organic layers were washed with brine, dried over sodium sulfate, filtered and concentrated. The residue was purified by prep-HPLC (0.05% of TFA in water with acetonitrile: 5% to 95%) to afford 136-7.

Step 3: To a mixture of 136-7 (32 mg, 0.041 mmol) in dichloromethane (2 mL) was added a solution of 0.1 M BCl₃ in dichloromethane (2 mL, 0.20 mmol) dropwise and the resulting mixture was stirred at 25° C. for 0.5 hour. The mixture was diluted with dichloromethane and washed with sat. aq. NaHCO₃ solution. The organic layer was washed with brine, dried over sodium sulfate, filtered and concentrated. The residue was purified by prep-HPLC (0.05% of FA in water with acetonitrile: 5% to 95%) to afford 136 as a 1.5 eq of FA salt. LCMS (ESI, m/z): [M+H]⁺=637.3. ¹H NMR (400 MHz, methanol-d₄, ppm): δ 8.50 (s, 1.5H), 7.63 (d, J=8.0 Hz, 1H), 7.35 (t, J=7.7 Hz, 1H), 7.30 (d, J=2.6 Hz, 1H), 7.20-6.92 (m, 3H), 5.44-5.30 (m, 1H), 4.45-4.35 (m, 2H), 3.82-3.64 (m, 4H), 3.51-3.38 (m, 4H), 3.17-3.10 (m, 1H), 2.48-2.43 (m, 1H), 2.38-2.29 (m, 2H), 2.26-2.17 (m, 2H), 2.11-2.05 (m, 2H), 2.01-1.92 (m, 1H), 1.89-1.71 (m, 2H), 1.70-1.44 (m, 2H), 1.38-1.24 (m, 1H), 0.86 (t, J=7.4 Hz, 3H).

### Example 47 Synthesis of Compound 142

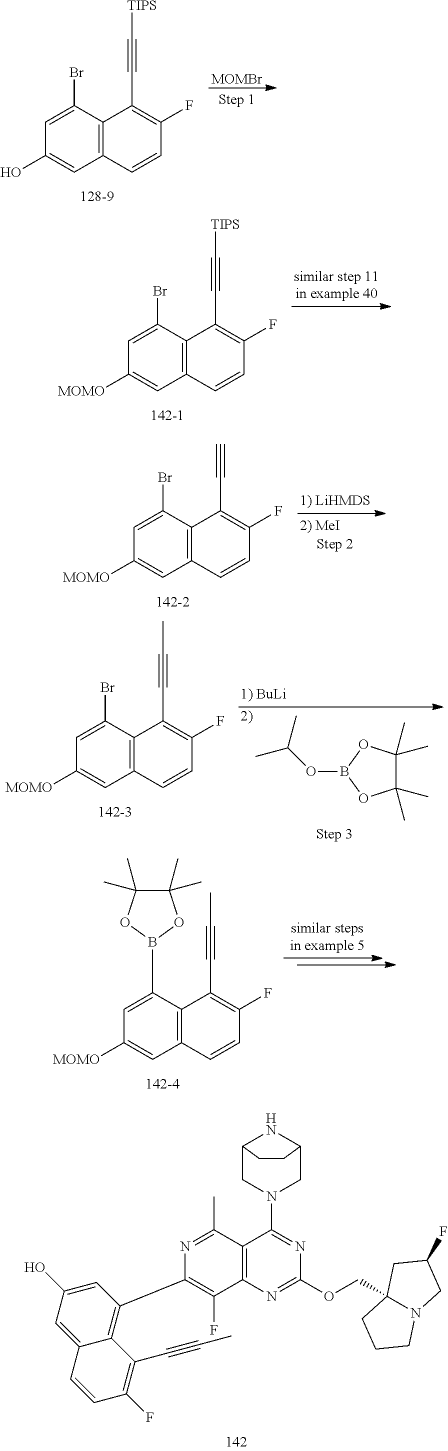

Step 1: To a solution of 128-9 (9.6 g, 23 mmol) in dichloromethane (100 mL) was added N, N-diisopropyl-ethylamine (4.45 g, 34.5 mmol) and bromomethyl methyl ether (3.45 g, 27.6 mmol) at room temperature, then stirred for 0.5 hour. The reaction mixture was washed with water, saturated aqueous sodium bicarbonate and brine. Organic layer was dried over sodium sulfate, filtered and concentrated to give 142-1.

Compound 142-2 was prepared from 142-1 following the procedure for the synthesis of compound 128-15 in example 40.

Step 2: To a solution of 142-2 (5.5 g, 18 mmol) in tetrahydrofuran (50 mL) was added lithium bis(trimethylsilyl)amide (23.4 mL, 23.4 mmol, 1 M in tetrahydrofuran) dropwise at −50° C. under N₂ atmosphere, then stirred for 15 minutes at −30° C. A solution of iodomethane (5.11 g, 36 mmol) in tetrahydrofuran (10 mL) was added to the mixture at −30° C. The reaction mixture was stirred for 15 minutes and quenched with water, diluted with ethyl acetate, washed with water and brine. Organic layer was dried over sodium sulfate, filtered and concentrated. The residue was purified by column chromatography on silica gel (petroleum ether to petroleum ether/ethyl acetate=4/1) to give 142-3.

Step 3: To a solution of 142-3 (5.47 g, 17 mmol) in tetrahydrofuran (50 mL) was added n-butyllithium (1.29 mL, 3.22 mmol, 2.5 M in hexane) at −78° C. over 10 minutes dropwise at −78° C. under N₂ atmosphere, then stirred for 15 minutes. To result mixture was added a solution of 2-isopropoxy-4,4,5,5-tetramethyl-1,3,2-dioxaborolane (4.74 g, 25.5 mmol) in tetrahydrofuran (5 mL) at −78° C., then stirred for 15 minutes. The reaction mixture was quenched with acetic acid, diluted with ethyl acetate, washed with water and brine. Organic layer was dried over sodium sulfate, filtered and concentrated. The residue was purified by column chromatography on silica gel (petroleum ether to petroleum ether/ethyl acetate=4/1) to give 142-4.

Compound 142 was prepared from 142-4 and 81-8 following the procedure for the synthesis of compound 60 in example 5 as a 3 eq of TFA salt. LCMS (ESI, m/z): [M+H]+=629.3; ¹H NMR (400 MHz, methanol-d₄, ppm): δ 7.78 (dd, J=9.2 Hz, 5.6 Hz, 1H), 7.33-7.28 (m, 2H), 7.13 (s, 1H), 5.65-5.52 (m, 1H), 4.87-4.81 (m, 1H), 4.76-4.69 (m, 2H), 4.34-4.20 (m, 2H), 4.18-3.85 (m, 6H), 3.51-3.44 (m, 1H), 2.78-2.55 (m, 5H), 2.48-2.31 (m, 3H), 2.21-1.95 (m, 4H), 1.91-1.73 (m, 1H), 1.31 (s, 3H). ¹⁹F NMR (376 MHz, methanol-d₄, ppm): δ −113.14 (1F), −143.45 (1F), −174.17 (1F).

### Example 48 Synthesis of Compound 144

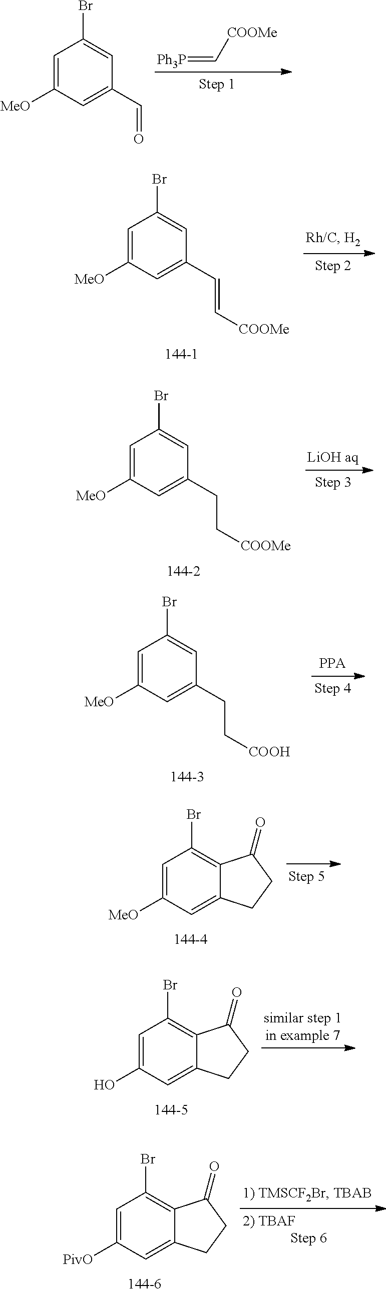

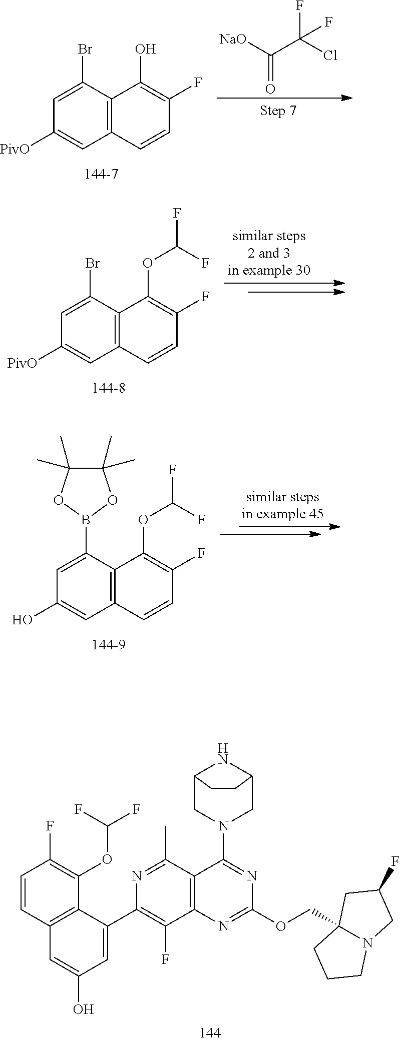

Step 1: A mixture of 3-bromo-5-methoxybenzaldehyde (100 g, 465 mmol) and methyl (triphenylphosphoranylidene)acetate (155 g, 465 mmol) in toluene (600 mL) was stirred for 3 hours at 25° C. The resulting mixture was filtered and washed with petroleum ether. The filtrate was concentrated and redissolved with petroleum ether/ethyl acetate (5/1) and stirred at 25° C. for 1 hour. The suspension was filtered and the filtrate was concentrated, dried under vacuum to give 144-1.

Step 2: A mixture of 144-1 (100 g, 370 mmol) and rhodium 10% on carbon (10 g) in methanol/tetrahydrofuran (1/1) (400 mL) was stirred for 16 hours at 45° C. The mixture was filtered and washed with ethyl acetate. The filtrate was concentrated and dried under vacuum to give 144-2.

Step 3: A mixture of 144-2 (120 g, crude) and lithium hydroxide (17.8 g, 740 mmol) in methanol/tetrahydrofuran/water (1/1/1) (600 mL) was stirred for 2 hours at 50° C. The resulting mixture was concentrated and diluted with water, adjusted to pH˜4 with 2 M hydrochloric acid aqueous. The mixture was filtered and washed with water. The filter cake was dried to afford 144-3.

Step 4: A solution of 144-3 (77 g, 298 mmol) in polyphosphoric acid (1000 g) was stirred for 2 hours at 100° C. The resulting mixture was poured into ice-water and extracted with dichloromethane. The combined organic layers were washed with water and saturated aqueous sodium bicarbonate. The organic layer was concentrated and purified by silica gel chromatography (dichloromethane to dichloromethane/ethyl acetate=20/1) to give 144-4.

Step 5: A solution of 144-4 (39 g, 162 mmol) in toluene (350 mL) was added aluminum chloride (34.4 g, 259 mmol) at 25° C. The resulting mixture was stirred at 105° C. for 4 hours under nitrogen atmosphere. The resulting mixture was cooled to room temperature and poured into ice-water. The suspension was filtered and washed with water. The filter cake was dried to afford 144-5.

Compound 144-6 was prepared from 144-5 following the procedure for the synthesis of compound 47-2 in example 7.

Step 6: A mixture of 144-6 (29 g, 93 mmol) and tetrabutylammonium bromide (3 g, 9.3 mmol) in toluene (300 mL) was added [bromo(difluoro)methyl]-trimethylsilane (75 g, 374 mmol) at 25° C. The resulting mixture was stirred at 110° C. for 3 hours. The resulting mixture was cooled to room temperature and added 1 M tetrabutylammonium fluoride (28 mL, 28 mmol), then stirred for 2 hours. The solution was diluted with water and extracted with dichloromethane. The combined organic layers were concentrated and purified by column chromatography on silica gel (petroleum ether to petroleum ether/ethyl acetate=9/1) to give 144-7.

Step 7: A mixture of 144-7 (3 g, 8.8 mmol) and potassium carbonate (1.82 g, 13.2 mmol) in N,N-dimethylformamide (15 mL) was added sodium 2-chloro-2,2-difluoroacetate (2 g, 13.2 mmol) at 25° C. The resulting mixture was stirred at 70° C. for 4 hours. The resulting mixture was diluted with water and extracted with dichloromethane. The combined organic layers were concentrated and purified by column chromatography on silica gel (petroleum ether to petroleum ether/ethyl acetate=20/1) to give 144-8.

Compound 144-9 was prepared from 144-8 following the procedure for the synthesis of compound 106-3 in example 30.

Compound 144 was prepared from 144-9 following the procedure for the synthesis of compound 139 in example 45 as a 4 eq of TFA salt. LCMS (ESI, m/z): [M+H]⁺=657.2; ¹H NMR (400 MHz, methanol-d₄, ppm): δ 7.76 (dd, J=9.2, 5.2 Hz, 1H), 7.45-7.30 (m, 3H), 6.67-6.28 (m, 1H), 5.66-5.52 (m, 1H), 4.90-4.88 (m, 1H), 4.77-4.64 (m, 2H), 4.29-4.04 (m, 4H), 4.03-3.81 (m, 4H), 3.55-3.40 (m, 1H), 2.92-2.57 (m, 5H), 2.55-2.14 (m, 5H), 2.12-1.91 (m, 3H); ¹⁹F NMR (376 MHz, methanol-d₄, ppm): δ −83.42 (2F), −135.35 (1F), −145.81 (1F), −174.13 (1F).

### Example 49 Synthesis of Compound 145

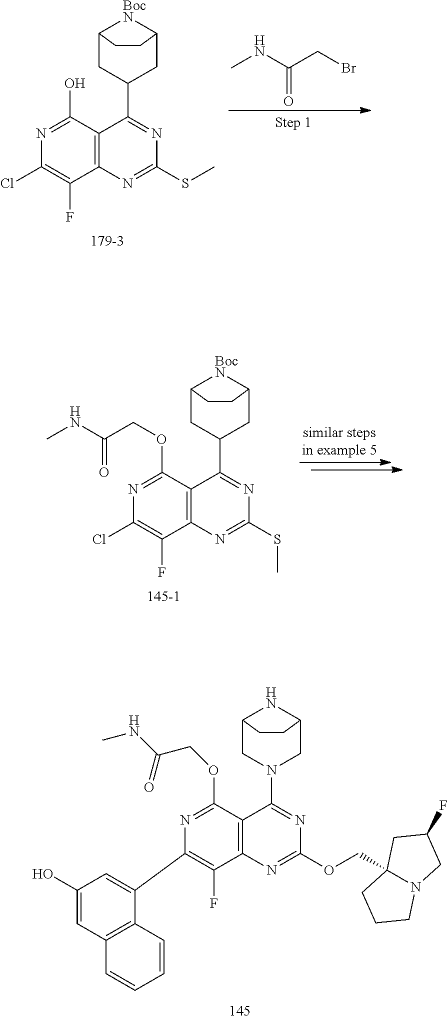

Step 1: A mixture of 179-3 (600 mg, 1.316 mmol) and 2-bromo-N-methylacetamide (260.02 mg, 1.711 mmol) and potassium carbonate (454.71 mg, 3.290 mmol) in N,N-dimethylformamide (12 mL) was stirred at 70° C. for 5 hours under nitrogen. The reaction was diluted with ethyl acetate and water. The organic layer was separated, washed with brine, dried over sodium sulfate, filtered and concentrated. The residue was purified using silica gel column chromatography (eluting with ethyl acetate in petroleum ether 2:1) to afford 145-1.

Compound 145 was prepared from 145-1 and 60-1 following the procedure for the synthesis of compound 139 in example 45 as a 3 eq of TFA salt. LCMS (ESI, m/z): [M+H]⁺-646.4; ¹H NMR (400 MHz, methanol-d₄, ppm): δ 7.74-7.72 (m, 1H), 7.63-7.60 (m, 1H), 7.43-7.39 (m, 1H), 7.25-7.19 (m, 3H), 5.64-5.51 (m, 1H), 4.97 (s, 2H), 4.76-4.67 (m, 4H), 4.26-4.17 (m, 2H), 4.08-3.84 (m, 3H), 3.79-3.75 (m, 2H), 3.51-3.44 (m, 1H), 2.77-2.55 (m, 5H), 2.46-2.31 (m, 3H), 2.23-2.11 (m, 5H). ¹⁹F NMR (376 MHz, methanol-d₄, ppm): δ −148.63 (1F), −174.19 (1F).

### Example 50 Synthesis of Compound 152

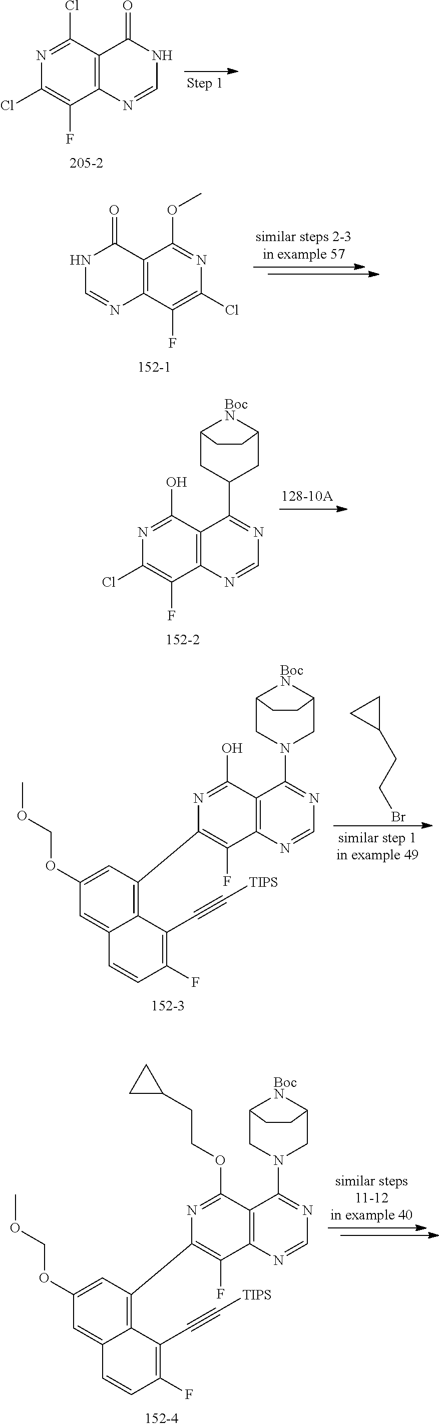

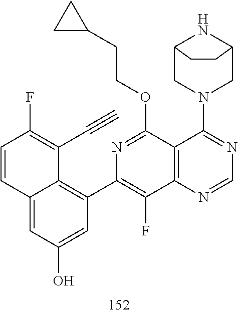 [SMILES: C#Cc1c(F)ccc2cc(O)cc(-c3nc(OCCC4CC4)c4c(N5CC6CCC(C5)N6)ncnc4c3F)c12]

Step 1: To a solution of 205-2 (4.5 g, crude) in N,N-dimethylacetamide (126 mL) and methanol (21 mL) was added sodium methanolate (5.19 g, 96.1 mmol) and then stirred for 1 hour. The reaction mixture was poured into water, adjusted to pH=2-3 with conc. hydrochloride, diluted with ethyl acetate, washed with water and brine. The organic layers were dried over sodium sulfate and concentrated to afford 152-1.

Compound 152-2 was prepared from 152-1 following the procedure for the synthesis of compound 179-3 in example 57.

Compound 152-3 was prepared from 152-2 and 128-10A following the procedure for the synthesis of compound 155-7 in example 53.

Compound 152-4 was prepared from 152-4 and (bromomethyl)cyclopropane following the procedure for the synthesis of compound 145-1 in example 49.

Compound 152 was prepared from 152-4 following the procedure for the synthesis of compound 128 in example 40. LCMS (ESI, m/z): [M+H]⁺=528.3; ¹H NMR (400 MHz, DMSO-d₆, ppm): δ 10.14 (br s, 1H), 8.50 (s, 1H), 7.93 (dd, J=9.2, 6.0 Hz, 1H), 7.47-7.38 (m, 1H), 7.35 (d, J=2.4 Hz, 1H), 7.20 (d, J=2.4 Hz, 1H), 4.38-3.97 (m, 4H), 3.76 (s, 1H), 3.44-3.40 (m, 4H), 1.66-1.38 (m, 6H), 0.74-0.60 (m, 1H), 0.39-0.25 (m, 2H), 0.06-0.08 (m, 2H). ¹⁹F NMR (376 MHz, DMSO-d₆, ppm): δ −110.47 (1F), −149.27 (1F).

### Example 51 Synthesis of Compound 153

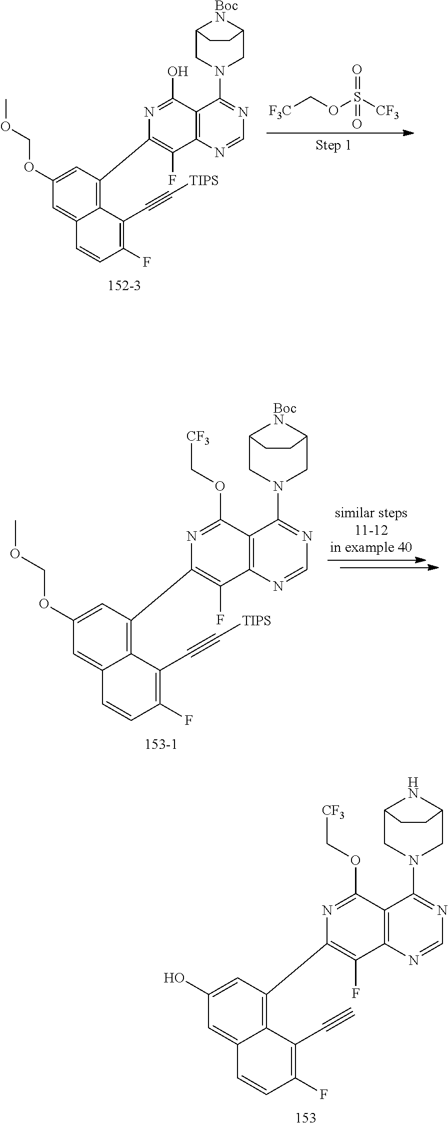

Step 1: To a mixture of 152-3 (400 mg, 0.527 mmol) in N,N-dimethylformamide (30 mL) was added 2,2,2-trifluoroethyl trifluoromethanesulfonate (366 mg, 1.58 mmol) and potassium carbonate (363 mg, 2.63 mmol). The mixture was stirred at 70° C. for 3 hours under Nitrogen atmosphere and diluted with ethyl acetate and water. The organic layer was separated, washed with brine, dried over sodium sulfate, filtered and concentrated to give 153-1.

Compound 153 was prepared from 153-1 following the procedure for the synthesis of compound 128 in example 40. LCMS (ESI, m/z): [M+H]⁺=542.2; ¹H NMR (400 MHz, methanol-d₄, ppm): δ 8.56 (s, 1H), 7.85 (dd, J=9.2, 5.6 Hz, 1H), 7.38-7.28 (m, 2H), 7.25 (d, J=2.4 Hz, 1H), 5.06-4.87 (m, 2H), 4.52-4.18 (m, 2H), 3.66-3.53 (m, 4H), 1.77-1.51 (m, 4H). ¹⁹F NMR (376 MHz, methanol-d₄, ppm): δ −74.35 (3F), −111.55 (1F), −147.64 (1F).

### Example 52 Synthesis of Compound 171

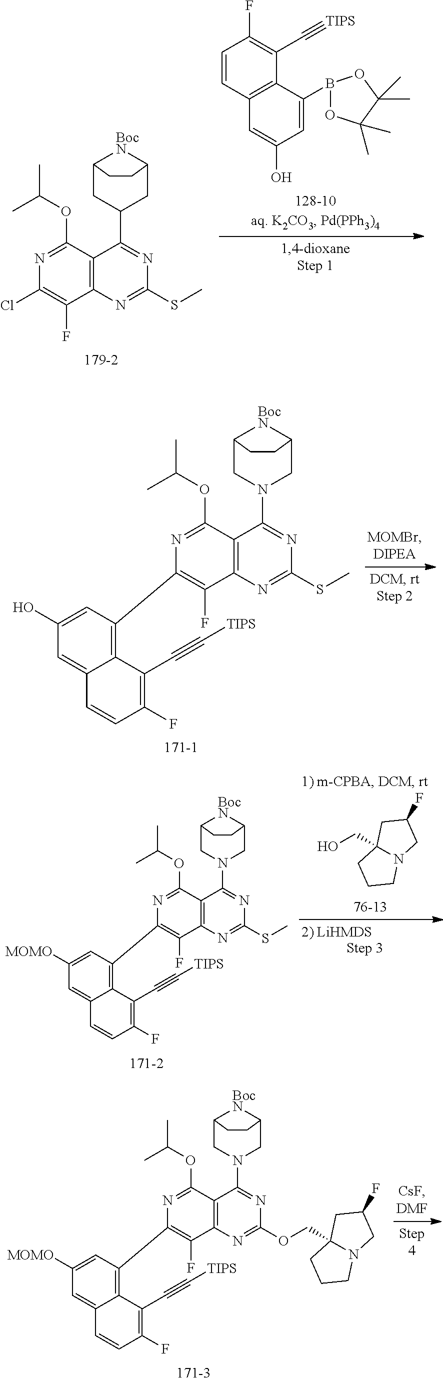

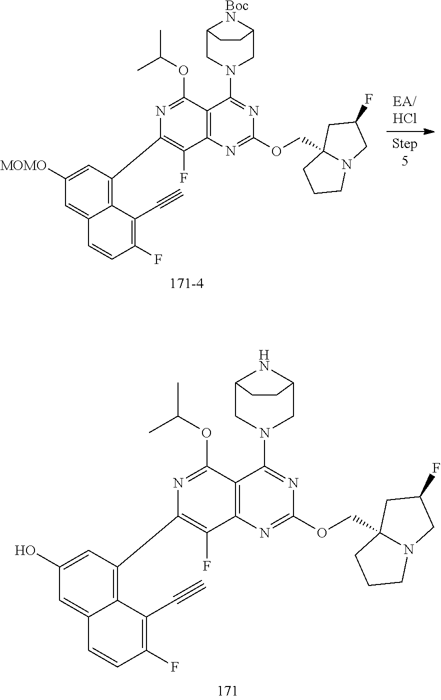

Step 1: A mixture of 179-2 (498 mg, 1.0 mmol), 128-10 (412 mg, 1.5 mmol), potassium carbonate (552 mg, 4.0 mmol) and tetrakis(triphenylphosphine)palladium (116 mg, 0.1 mmol) in 1,4-dioxane/water (4/1, 10 mL) was stirred at 135° C. for 1 hour under nitrogen atmosphere with microwave. After the mixture was cooled to room temperature, it was diluted with ethyl acetate, washed with water and brine. Organic layer was dried over sodium sulfate, filtered and concentrated. The residue was purified by column chromatography on silica gel (petroleum ether to petroleum ether/ethyl acetate=2/1) to give 171-1 as a yellow solid.

Step 2: To a solution of 171-1 (700 mg, 0.87 mmol) in dichloromethane (10 mL) was added N, N-diisopropyl-ethylamine (337 mg, 2.61 mmol) and bromo(methoxy)methane (131 mg, 1.04 mmol) at room temperature, then stirred for 0.5 hours. The mixture was diluted with dichloromethane, washed with saturated sodium bicarbonate aqueous and brine. Organic layer was dried over sodium sulfate, filtered and concentrated. The residue was purified by column chromatography on silica gel (petroleum ether to petroleum ether/ethyl acetate=4/1) to give 171-2 as a yellow solid.

Step 3: To a solution of 171-2 (400 mg, 0.47 mmol) in dichloromethane (8 mL) was added 3-chloroperbenzoic acid (115 mg, 0.57 mmol) at 0° C., then stirred for 0.5 hours. Diluted with dichloromethane, washed with saturated sodium bicarbonate aqueous and brine. Organic layer was dried over sodium sulfate, filtered and concentrated to give a crude intermediate which was dissolved in anhydrous tetrahydrofuran (4 mL) and added into a stirred solution of 76-13 (113 mg, 0.277 mmol) and lithium bis(trimethylsilyl)amide (0.66 mL, 0.66 mmol, 1 M in tetrahydrofuran) in tetrahydrofuran (5 mL) at 0° C. under nitrogen atmosphere, then stirred for 0.5 hours. The mixture was diluted with ethyl acetate, washed with water and brine. Organic layer was dried over sodium sulfate, filtered and concentrated. The residue was purified by prep-HPLC (acetonitrile/0.05% TFA in water: 20%˜95%) to give 171-3 as an off-white solid.

Step 4: To a solution of 171-3 (280 mg, 0.29 mmol) in N, N-dimethylformamide (6 mL) was added cesium fluoride (444 mg, 2.09 mmol) at room temperature, then stirred for 2 hours. The mixture was diluted with ethyl acetate, washed with water and brine. Organic layer was dried over sodium sulfate, filtered and concentrated to give 171-4 as yellow oil.

Step 5: A solution of 171-4 (220 mg, 0.27 mmol) in ethyl acetate (12 mL, 1.0 M HCl) stirred for 1 hour at 50° C. The mixture was diluted with ethyl acetate and saturated sodium bicarbonate aqueous, extracted with ethyl acetate. Organic layer was dried over sodium sulfate, filtered and concentrated. The residue was purified by prep-HPLC (acetonitrile/0.05% TFA in water: 10%˜95%) to give 171 as an off-white solid as a 3.0 eq of TFA salt. LCMS (ESI, m/z): [M+H]⁺=659.3; ¹H NMR (400 MHz, methanol-d₄, ppm): δ 7.89-7.81 (m, 1H), 7.37-7.26 (m, 2H), 7.19-7.17 (m, 1H), 5.66-5.48 (m, 2H), 4.74-4.46 (m, 4H), 4.26-4.14 (m, 2H), 4.10-3.81 (m, 5H), 3.50-3.42 (m, 1H), 3.40-3.35 (m, 1H), 2.76-2.52 (m, 2H), 2.47-2.28 (m, 3H), 2.20-1.95 (m, 5H), 1.45-1.36 (m, 6H). ¹⁹F NMR (376 MHz, methanol-d₄, ppm): δ −111.8 (1F), −151.5 (1F), −174.2 (1F).

### Example 53 Synthesis of Compound 155 and 156

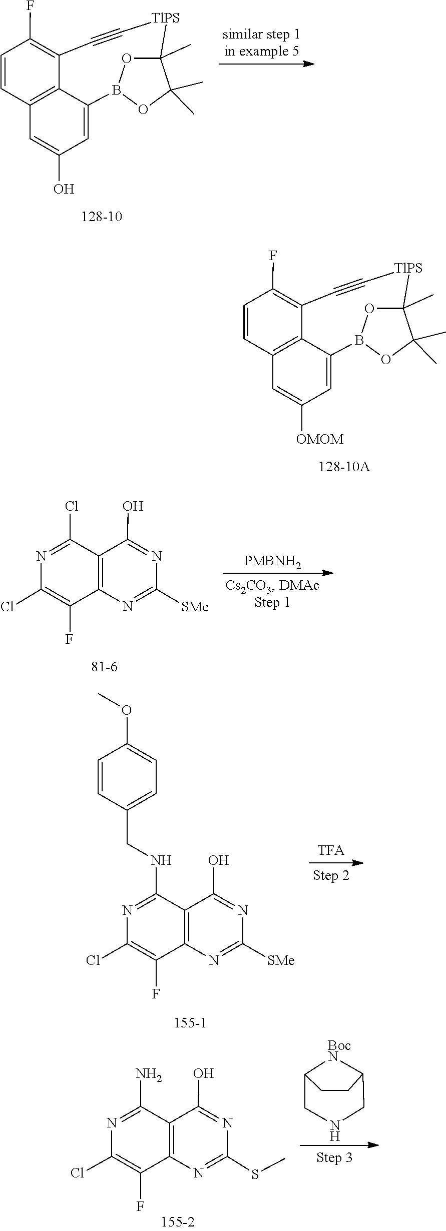

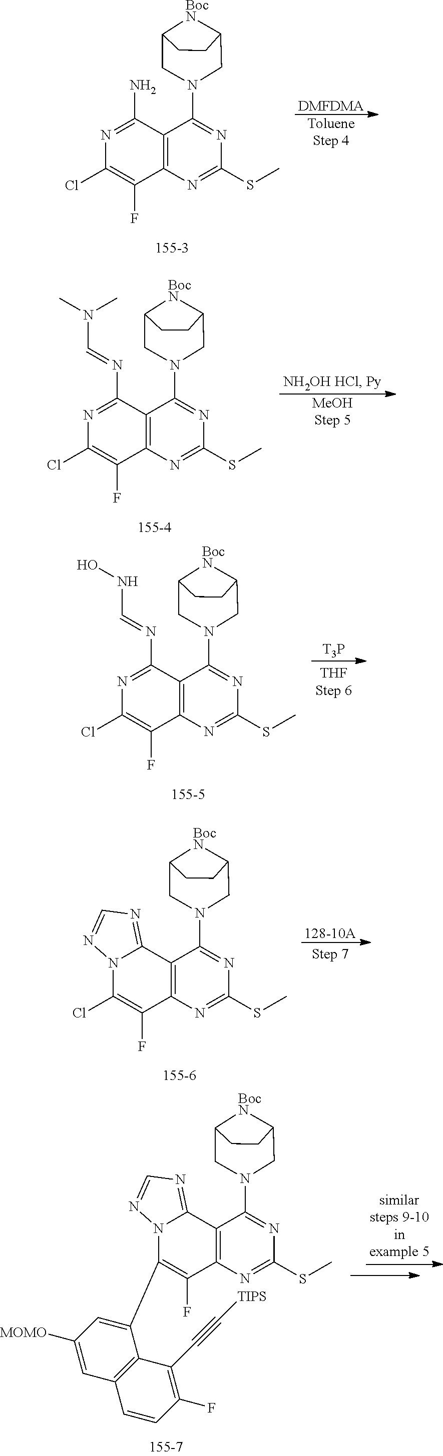

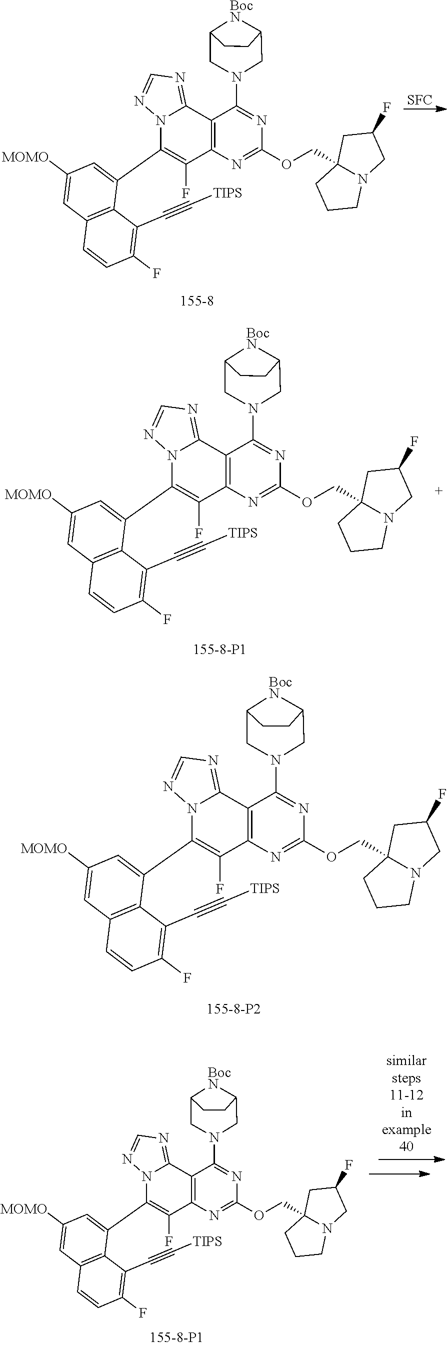

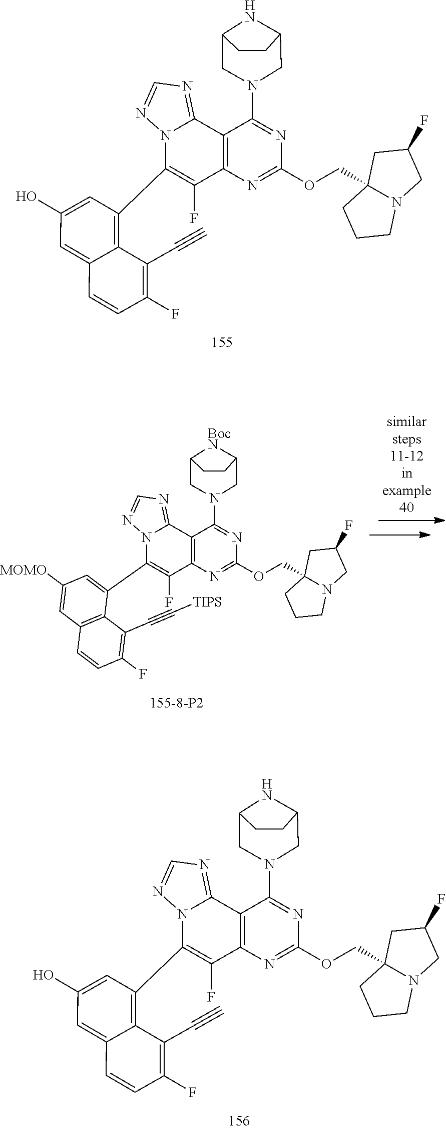

Compound 128-10A was prepared from 128-10 following the procedure for the synthesis of compound 60-1 in example 5.

Step 1: A mixture of 81-6 (2.80 g, 9.996 mmol), (4-methoxyphenyl) methanamine (4.11 g, 29.990 mmol) and cesium carbonate (9.75 g, 0.600 mmol) in N,N-dimethylacetamide (50 mL) was stirred at 100° C. for 16 hours. After cooled to room temperature, the reaction mixture was poured into water, filtered and washed with water. The filter cake was slurried with methanol to give 155-1.

Step 2: A mixture of 155-1 (3.45 g, 9.059 mmol) in trifluoroacetic acid (20 mL) was stirred at 30° C. for 2 hours. The mixture was poured into ice-water, filtered and washed with water. The filter cake was collected and dried to give 155-2, which was used in the next step without purification.

Step 3: To a solution of 155-2 (2.8 g, from step 2) in N,N-dimethylformamide (30 mL) was added benzotriazole-1-yl-oxytripyrrolidinophosphonium hexafluorophosphate (10.78 g, 20.715 mmol), tert-butyl 3,8-diazabicyclo[3.2.1]octane-8-carboxylate (4.40 g, 20.715 mmol), and 1,8-diazabicyclo[5.4.0]undec-7-ene (3.15 g, 20.715 mmol) at 0° C., then stirred for 16 hours at room temperature. The mixture was poured into water, filtered and washed with water. The filter cake was slurried with acetonitrile/water (10 mL/10 mL) to give 155-3.

Step 4: A mixture of 155-3 (2.3 g, 5.055 mmol) and N,N-dimethylformamide dimethyl acetal (1.8 g, 15.166 mmol) in toluene (40 mL) was stirred for 1 hour at 100° C. The mixture was concentrated to give 155-4, which was used in next step without purification.

Step 5: A mixture of 155-4 (5 mmol, from step 4), pyridine (1186.5 mg, 15.000 mmol) and hydroxylamine hydrochloride (694.9 mg, 10.000 mmol) in methanol (50 mL) was stirred for 2 hours at room temperature. The reaction mixture was diluted with water, filtered and washed with water. The filter cake was slurried with acetonitrile/water, filtered. The filter cake was collected and dried to give 155-5.

Step 6: To a solution of 155-5 (2 g, 4.016 mmol) in tetrahydrofuran (40 mL) was added propylphosphonic acid anhydride (1277.9 mg, 4.016 mmol, 50% wt in ethyl acetate) at room temperature under N₂, then stirred for 4 hours. The reaction mixture was diluted with ethyl acetate, washed with saturated aqueous sodium bicarbonate and brine, dried over sodium sulfate, filtered and concentrated. The residue was purified with column chromatography on silica gel (petroleum ether to petroleum ether/ethyl acetate=3/1) to give 155-6.

Step 7: A mixture of 155-6 (383.98 mg, 0.8 mmol), 128-10A (492.05 mg, 0.960 mmol), potassium carbonate (331.7 mg, 2.400 mmol) and tetrakis(triphenylphosphine)palladium (92.4 mg, 0.080 mmol) in dioxane (15 mL) and water (3 mL) was stirred at 125° C. for 0.5 hours under N₂ atmosphere with microwave condition. The reaction mixture was diluted with ethyl acetate, washed with water and brine, dried over sodium sulfate, filtered and concentrated. The residue was purified by column chromatography on silica gel (petroleum ether to petroleum ether/ethyl acetate=4/1) to give 155-7.

Compound 155-8 was prepared from 155-7 following the procedure for the synthesis of compound 60-10 in example 5.

Compound 155-8 was purified by chiral prep-HPLC (column: CHIRALPAK®IA, 30% IPA in hexane) to afford 155-8-P1 and 155-8-P2, respectively.

155-8-P1: Chiral SFC analysis: 99.28% ee. Retention time 2.792 min on REGIS (S,S)WHELK-O1 50*4.6 mm 3 mm column (35° C.); mobile phase: CO₂/methanol (+0.1% DEA), 1.5 mL/min, 1800 psi.

155-8-P2: Chiral SFC analysis: 97.32% ee. Retention time 3.681 min on REGIS (S,S)WHELK-O1 50*4.6 mm 3 mm column (35° C.); mobile phase: CO₂/methanol (+0.1% DEA), 1.5 mL/min, 1800 psi.

Compound 155 was prepared from 155-8-P1 following the procedure for the synthesis of compound 128 in example 40 as a 3 eq of TFA salt.

155: LCMS (ESI, m/z): [M+H]⁺=641.4; ¹H NMR (400 MHz, methanol-d₄, ppm): δ 8.34 (s, 1H), 7.95-7.90 (m, 1H), 7.51-7.47 (m, 1H), 7.43-7.32 (m, 2H), 5.71-5.49 (m, 1H), 4.79-4.63 (m, 4H), 4.35-4.23 (m, 2H), 4.09-3.82 (m, 3H), 3.76-3.57 (m, 2H), 3.54-3.43 (m, 1H), 3.05 (s, 1H), 2.83-2.53 (m, 4H), 2.49-2.31 (m, 3H), 2.29-2.07 (m, 3H). ¹⁹F NMR (376 MHz, methanol-d₄, ppm): δ −110.81 (1F), −149.95 (1F), −174.13 (1F).

Compound 156 was prepared from 155-8-P2 following the procedure for the synthesis of compound 128 in example 40 as a 2 eq of TFA salt.

156: LCMS (ESI, m/z): [M+H]⁺=641.4; ¹H NMR (400 MHz, methanol-d₄, ppm): δ 8.34 (s, 1H), 7.95-7.90 (m, 1H), 7.49-7.48 (m, 1H), 7.41-7.32 (m, 2H), 5.68-5.51 (m, 1H), 4.79-4.63 (m, 4H), 4.32-4.13 (m, 2H), 4.09-3.82 (m, 3H), 3.73-3.41 (m, 3H), 3.03 (s, 1H), 2.83-2.53 (m, 4H), 2.49-2.12 (m, 6H). ¹⁹F NMR (376 MHz, methanol-d₄, ppm): δ −110.79 (1F), −149.95 (1F), −174.16 (1F).

### Example 54 Synthesis of Compound 165

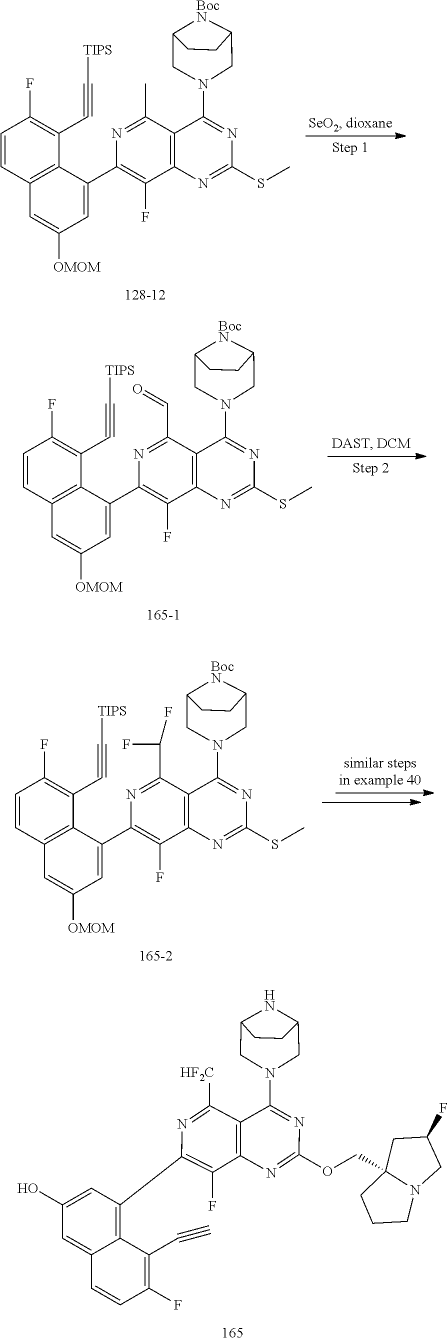

Step 1: To a solution of 128-12 (540 mg, 0.67 mmol) in dioxane (10 mL) was added selenium dioxide (127 mg, 1.14 mmol) at room temperature, then stirred at 110° C. for 2 hours under nitrogen atmosphere. The reaction mixture was diluted with dichloromethane, washed with water and brine. Organic layer was dried over sodium sulfate, filtered and concentrated. The residue was purified by column chromatography on silica gel (petroleum ether to petroleum ether/ethyl acetate=4/1) to give 165-1.

Step 2: To a solution of 165-1 (260 mg, 0.32 mmol) in dichloromethane (10 mL) was added N,N-diethyl-1,1,1-trifluoro-14-sulfanamine (768 mg, 4.77 mmol) at room temperature, then stirred at 30° C. for 16 hours under nitrogen atmosphere. The reaction mixture was diluted with dichloromethane, washed with water and brine. Organic layer was dried over sodium sulfate, filtered and concentrated. The residue was purified by column chromatography on silica gel (petroleum ether to petroleum ether/ethyl acetate=4/1) to give 165-2.

Compound 165 was prepared from 165-2 following the procedure for the synthesis of compound 128 in example 40 as a 4 eq of TFA salt. LCMS (ESI, m/z): [M+H]⁺=651.4; ¹H NMR (400 MHz, methanol-d₄, ppm): δ 7.89 (dd, J=9.2, 6.0 Hz, 1H), 7.40-7.30 (m, 2H), 7.25-6.88 (m, 2H), 5.73-5.50 (m, 1H), 4.85-4.82 (m, 1H), 4.79-4.49 (m, 3H), 4.32-4.15 (m, 2H), 4.10-3.82 (m, 5H), 3.54-3.41 (m, 1H), 3.29-3.24 (m, 1H), 2.82-2.50 (m, 2H), 2.48-2.28 (m, 3H), 2.26-1.73 (m, 5H). ¹⁹F NMR (376 MHz, methanol-d₄, ppm): δ −82.35 (2F), −111.52 (1F), −136.69 (1F), −174.19 (1F).

### Example 55 Synthesis of Compound 170

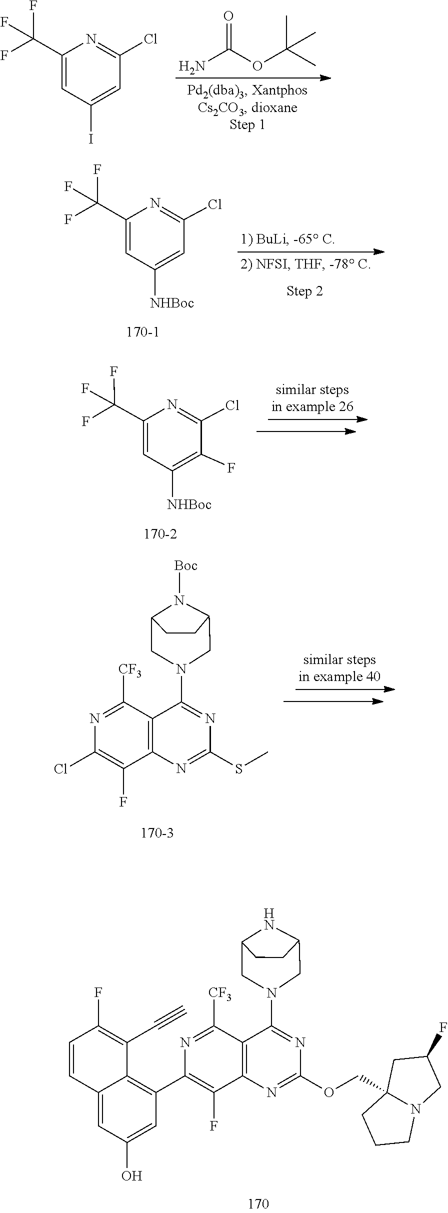

Step 1: To a solution of 2-chloro-4-iodo-6-(trifluoromethyl)pyridine (24.5 g, 80 mmol), tert-butyl carbamate (11.23 g, 96 mmol), cesium carbonate (39.12 g, 120 mmol), tris(dibenzylideneacetone)dipalladium (2.93 g, 3.2 mmol), and 4,5-bis(diphenylphosphino)-9,9-dimethylxanthene (3.7 g, 6.4 mmol) in 1,4-dioxane (250 mL) was stirred at 100° C. for 5 hours under N₂ atmosphere. After cooling to room temperature, the reaction solution was diluted with ethyl acetate, washed water, brine, dried over sodium sulfate, filtered and concentrated. The residue was purified by column chromatography on silica gel (petroleum ether to petroleum ether/ethyl acetate=3/1) to give crude product, which was slurried with petroleum ether to give 170-1.

Step 2: To a solution of 170-1 (14.8 g, 50 mmol) in tetrahydrofuran (100 mL) was added n-butyllithium (56 mL, 140 mmol) dropwised at −65° C. under N₂ atmosphere, then stirred for 4 hours. A solution of N-fluorobenzenesulfonimide (18.9 g, 60 mmol) in tetrahydrofuran (80 mL) was added dropwised to the above mixture at −78° C. under N₂ atmosphere, then stirred for 0.5 hour. The reaction was quenched with acetic acid, diluted with ethyl acetate, washed with water and brine. Organic layer was dried over sodium sulfate, filtered and concentrated. The residue was purified by column chromatography on silica gel (petroleum ether to petroleum ether/ethyl acetate=4/1) to give 170-2.

Compound 170-3 was prepared from 170-2 following the procedure for the synthesis of compound 81-7 in example 26.

Compound 170 was prepared from 170-3 following the procedure for the synthesis of compound 128 in example 40 as a 4 eq of TFA salt. LCMS (ESI, m/z): [M+H]⁺=669.3; ¹H NMR (400 MHz, methanol-d₄, ppm): δ 7.89 (dd, J=9.2, 6.0 Hz, 1H), 7.40-7.32 (m, 2H), 7.28-7.24 (m, 1H), 5.65-5.51 (m, 1H), 4.82-4.63 (m, 4H), 4.28-4.16 (m, 2H), 4.06-3.84 (m, 5H), 3.51-3.43 (m, 1H), 3.36-3.32 (m, 1H), 2.79-2.53 (m, 2H), 2.47-2.31 (m, 3H), 2.28-1.99 (m, 3H), 1.96-1.57 (m, 2H). ¹⁹F NMR (376 MHz, methanol-d₄, ppm): δ −64.23 (3F), −111.38 (1F), −134.79 (1F), −174.16 (1F).

### Example 56 Synthesis of Compound 176

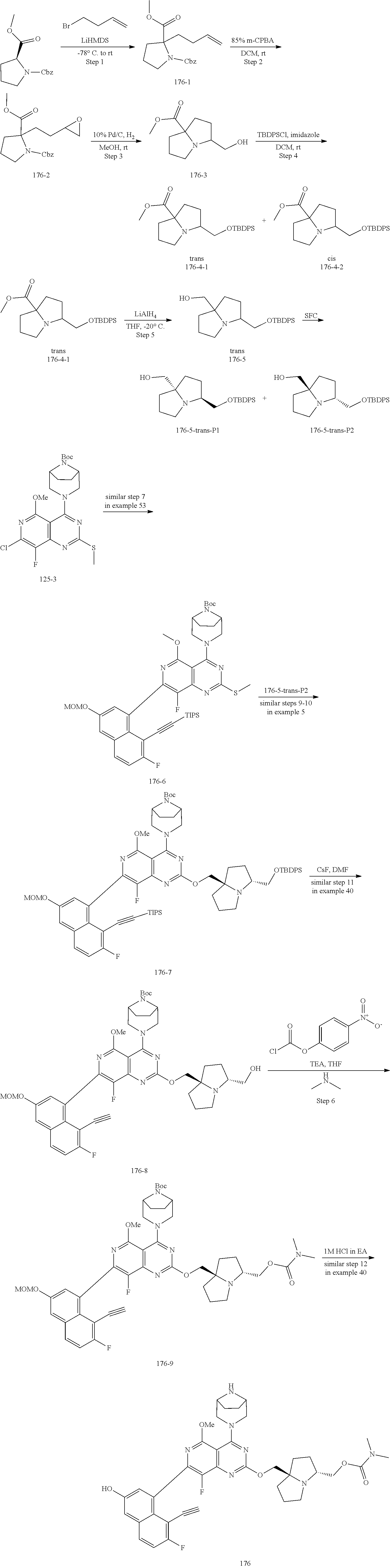

Step 1: To a mixture of benzyl (S)-2-(1-methoxyvinyl)pyrrolidine-1-carboxylate (95 g, 360.82 mmol) in tetrahydrofuran (400 mL) was added lithium bis(trimethylsilyl)amide (433 mL, 433 mmol, 1M in tetrahydrofuran) at −78° C., then stirred for 1 hour. Then 4-bromobut-1-ene (97.4 g, 721.64 mmol) was added at −78° C. The mixture was stirred at room temperature for 10 hours. Then the mixture was quenched with ammonium chloride aqueous and extracted with ethyl acetate. The combined organic layers were washed with brine, dried over sodium sulfate, filtered and concentrated. The residue was purified by column chromatography on silica gel (petroleum ether to petroleum ether/ethyl acetate=5/1) to give 176-1.

Step 2: To a solution of 176-1 (85.25 g, 268.6 mmol) in dichloromethane (1 L) wad added 3-chloroperbenzoic acid (136.33 g, 85%, 671.5 mmol) at 0° C., and the reaction was stirred at room temperature for 4 hours. The mixture was quenched with saturated sodium thiosulfate aqueous and extracted with dichloromethane. The organic layer was washed with saturated aqueous sodium bicarbonate and brine, dried over sodium sulfate, filtered and concentrated. The residue was purified by column chromatography on silica gel (petroleum ether to petroleum ether/ethyl acetate=3/1) to give 176-2.

Step 3: To a solution of 176-2 (64.5 g, 193.47 mmol) in methanol (300 mL) was added Pd/C (6.45 g). Then the mixture was stirred at room temperature for 24 hours. The mixture was filtered and concentrated to afford 176-3.

Step 4: To a mixture of 176-3 (38.55 g, 193.47 mmol) and imidazole (39.5 g, 580.41 mmol) in dichloromethane (700 mL) was added tert-butylchlorodiphenylsilane (79.77 g, 290.21 mmol) at 0° C. under N₂ atmosphere, then stirred for 1 hour at room temperature. Then the mixture was diluted with ice-water and extracted with ethyl acetate. The organic layer was washed with water and brine, dried over sodium sulfate, filtered and concentrated. The residue was purified by column chromatography on silica gel (petroleum ether to petroleum ether/ethyl acetate=2/1) to give 176-4-1 (trans) and 176-4-2 (cis).

Step 5: To a solution of 176-4-1 (trans) (34 g, 77.8 mmol) in tetrahydrofuran (500 mL) was added lithium aluminium hydride (6.1 g, 97%, 155.6 mmol) at −20° C. under nitrogen atmosphere, then stirred for 1 hour. Then the mixture was quenched with water, 15% sodium hydroxide aqueous and water at 0° C. The mixture was diluted with tetrahydrofuran, dried over sodium sulfate, filtered and concentrated to give 176-5 (trans).
176-5 (trans) (32 g, 78.115 mmol) was purified by preparative SFC [DAICELCHIRALPAK®IG with MEOH (+0.1% 7.0 mol/L Ammonia in methanol)/Supercritical CO₂] to give 176-5-trans-P1 and 176-5-trans-P2.
176-5-trans-P1: Chiral SFC analysis: >99.5% ee. Retention time 3.369 min on DAICELCHIRALPAK®IG 100*3 mm 3 μm column (35° C.); mobile phase: methanol (0.1% DEA) in CO₂, 1800 psi, 1.5 mL/min.
176-5-trans-P2: Chiral SFC analysis: 98.9% ee. Retention time 3.708 min on DAICELCHIRALPAK®IG 100*3 mm 3 μm column (35° C.); mobile phase: methanol (0.1% DEA) in CO₂, 1800 psi, 1.5 mL/min.

Compound 176-6 was prepared from 125-3 and 128-10A following the procedure for the synthesis of compound 155-7 in example 53.

Compound 176-7 was prepared from 176-6 and 176-5-trans-P2 following the procedure for the synthesis of compound 60-10 in example 5.

Compound 176-8 was prepared from 176-7 following the procedure for the synthesis of compound 128-15 in example 40.

Step 6: To a solution of 176-8 (100 mg, 0.127 mmol) and triethylamine (77 mg, 0.761 mmol) in tetrahydrofuran (2 mL) was added p-nitrophenylchloroformate (77 mg, 0.382 mmol) at room temperature, and the reaction was stirred at room temperature for 5 hours. Then dimethylamine (1.3 mL, 2M in tetrahydrofuran, 2.6 mmol) was added, and stirred at room temperature for 15 minutes. The mixture was diluted with ethyl acetate, washed with water and brine, dried over sodium sulfate, filtered and concentrated. The residue was purified by prep-TLC (dichloromethane/methanol=10/1) to give 176-9.

Compound 176 was prepared from 176-9 following the procedure for the synthesis of compound 128 in example 40 as a 3 eq of TFA salt. LCMS (ESI, m/z): [M+H]⁺=714.3; ¹H NMR (400 MHz, methanol-d₄, ppm): δ 7.89-7.82 (m, 1H), 7.38-7.27 (m, 2H), 7.24-7.21 (m, 1H), 4.75-4.61 (m, 2H), 4.54-4.19 (m, 7H), 4.06 (s, 3H), 3.79-3.69 (m, 2H), 3.67-3.56 (m, 1H), 3.49-3.33 (m, 2H), 2.98-2.82 (m, 6H), 2.49-2.30 (m, 2H), 2.29-2.01 (m, 10H). ¹⁹F NMR (376 MHz, methanol-d₄, ppm): δ −111.77 (1F), −150.31 (1F).

### Example 57 Synthesis of Compound 179

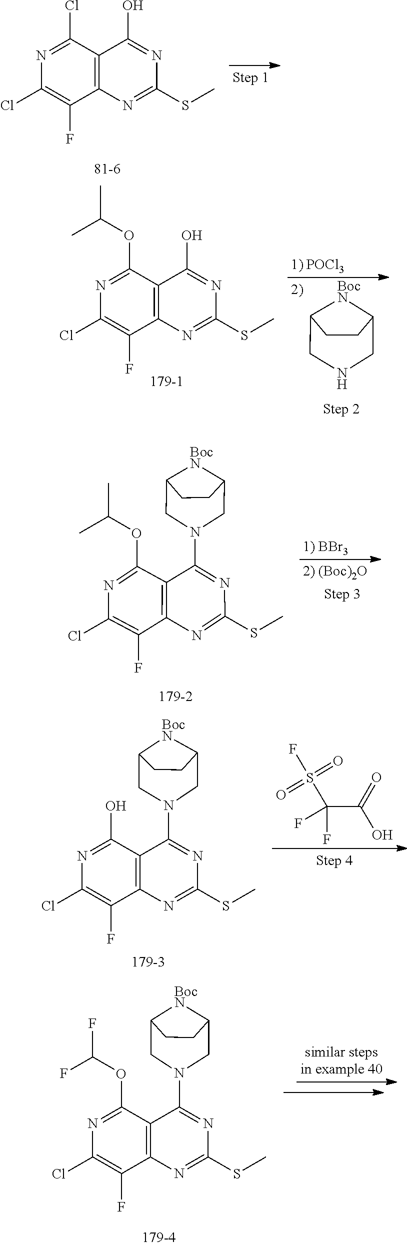

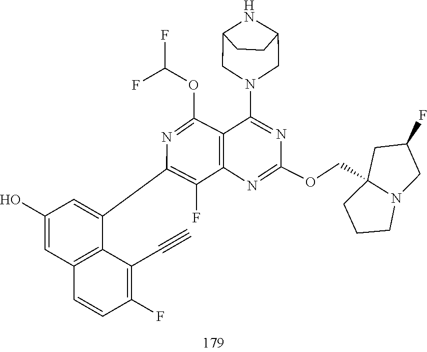 [SMILES: C#Cc1c(F)ccc2cc(O)cc(-c3nc(OC(F)F)c4c(N5CC6CCC(C5)N6)nc(OC[C@@]56CCCN5C[C@H](F)C6)nc4c3F)c12]

Step 1: To a mixture of propan-2-ol (1.8 g, 30 mmol) in dimethylacetamide (10 mL) was added lithium bis(trimethylsilyl)amide (30 mL, 30 mmol, 1 M in tetrahydrofuran) at 0° C., then stirred at 0° C. for 20 minutes. The mixture was added a solution of 81-6 (2.8 g, 10 mmol) in dimethylacetamide (20 mL) at 0° C., then stirred at 40° C. for 16 hours. The reaction mixture was diluted with water, adjusted to pH-4 with 2N hydrochloric acid, filtered and washed with water. The collected solids were dried to afford 179-1.

Step 2: A mixture of 179-1 (1.21 g, 4 mmol), N, N-diisopropyl-ethylamine (1.55 g, 12 mmol), phosphorus oxychloride (1.22 g, 8 mmol) in acetonitrile (10 mL) was stirred at 80° C. for 40 mins under nitrogen atmosphere. After cooled to 0° C., N, N-diisopropyl-ethylamine (1.55 g, 13 mmol) and tert-butyl (1R,5S)-3,8-diazabicyclo[3.2.1]octane-8-carboxylate (1.27 g, 6 mmol) was added, then stirred at room temperature for 1 hour. The reaction mixture was diluted with ethyl acetate, washed with water and brine. Organic layer was dried over sodium sulfate, filtered and concentrated to give a residue which was purified by column chromatography on silica gel (petroleum ether to petroleum ether/ethyl acetate=5/1) to give 179-2.

Step 3: 179-2 (5 g, 10 mmol) in dichloromethane (100 mL) was added boron tribromide (30 mL, 30 mmol, 1 M in dichloromethane) at 0° C., stirred at room temperature for 16 hours. The resulting mixture was filtered and the filter cake was dissolved with tetrahydrofuran (50 mL). To above mixture was added triethylamine (5.1 g, 50 mmol) and di-tert-butyl decarbonate (3.3 g, 15 mmol), then stirred at room temperature for 2 hours. The resulting mixture was filtered and the filtrate was concentrated to give 179-3.

Step 4: To a solution of 179-3 (1.8 g, 3.95 mmol) in acetonitrile (40 mL) was added 60% sodium hydride (427 mg, 10.66 mmol) at room temperature, then 2,2-difluoro-2-(fluorosulfonyl) acetic acid (1.19 g, 6.71 mmol) was added. The solution was stirred at 30° C. for 5 hours under nitrogen atmosphere. The reaction mixture was diluted with water and extracted with ethyl acetate. The combined organic layers were concentrated and purified by column chromatography on silica gel (petroleum ether to petroleum ether/ethyl acetate=4/1) to give 179-4.

Compound 179 was prepared from 179-4 and 128-10A following the procedure for the synthesis of compound 128 in example 40 as a 3 eq of TFA salt. LCMS (ESI, m/z): [M+H]⁺-667.3; ¹H NMR (400 MHz, methanol-d₄, ppm): δ 7.94-7.48 (m, 2H), 7.39-7.28 (m, 2H), 7.25-7.20 (m, 1H), 5.70-5.44 (m, 1H), 4.78-4.64 (m, 2H), 4.62-4.47 (m, 2H), 4.28-4.19 (m, 2H), 4.09-3.80 (m, 5H), 3.54-3.38 (m, 2H), 2.84-2.53 (m, 2H), 2.48-2.28 (m, 3H), 2.26-2.04 (m, 5H). ¹⁹F NMR (376 MHz, methanol-d₄, ppm): δ −90.74 (2F), −111.50 (1F), −145.35 (1F), −174.18 (1F).

### Example 58 Synthesis of Compound 183

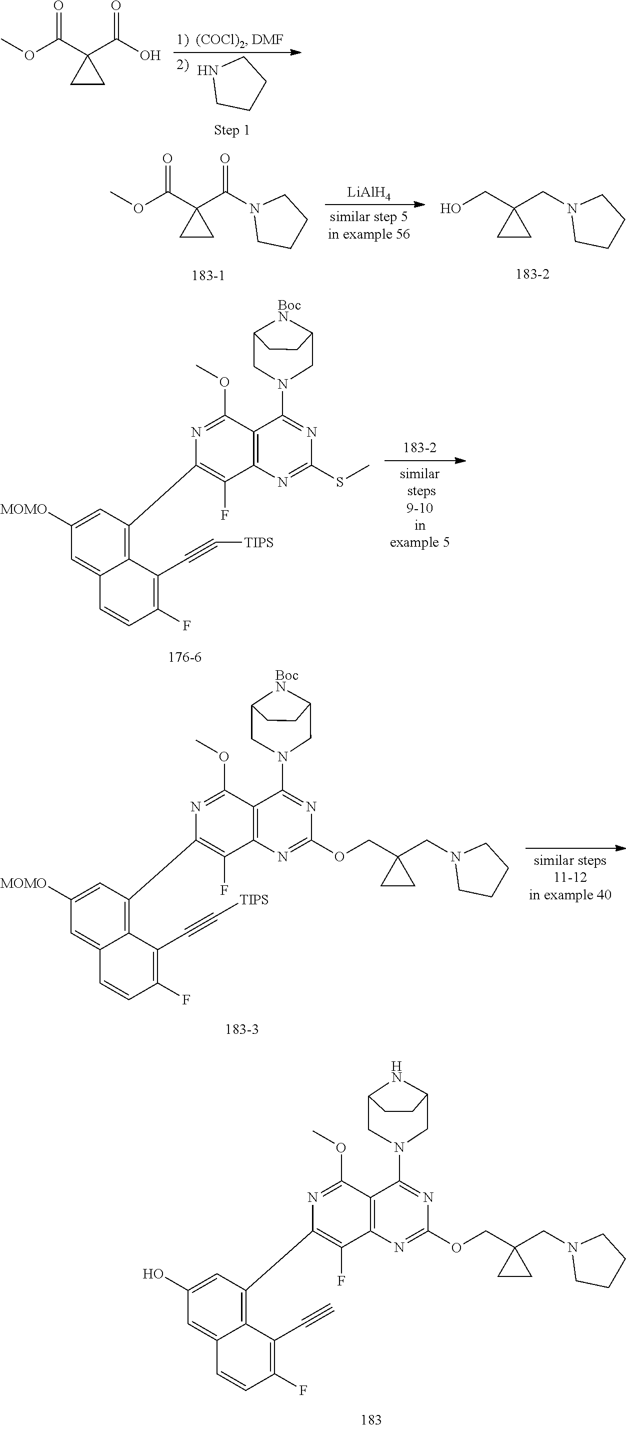

Step 1: To a solution of 1-(methoxycarbonyl)cyclopropane-1-carboxylic acid (2.88 g, 19.99 mmol) in dichloromethane (50 mL) were added oxalyl chloride (6.86 mL, 79.93 mmol) and N, N-dimethylformamide (0.05 mL) at 0° C., and the reaction was stirred at 35° C. for 3 hours. The reaction was concentrated to give a solution. The residue was dissolved in dichloromethane (80 mL) and added triethylamine (8.33 mL, 59.95 mmol), pyrrolidine (1.97 mL, 23.98 mmol) at 0° C., and the reaction was stirred at room temperature for 3 hours, filtered and concentrated. The residue was purified using silica gel column chromatography (eluting with methanol in dichloromethane=1:20) to afford 183-1.

Compound 183-2 was prepared from 183-1 following the procedure for the synthesis of compound 176-5 in example 56.

Compound 183-3 was prepared from 176-6 and 183-2 following the procedure for the synthesis of compound 60-10 in example 5.

Compound 183 was prepared from 183-3 following the procedure for the synthesis of compound 128 in example 40 as a 4 eq of TFA salt. LCMS (ESI, m/z): [M+H]⁺=627.4; ¹H NMR (400 MHz, methanol-d₄, ppm): δ 7.89-7.82 (m, 1H), 7.38-7.28 (m, 2H), 7.27-7.18 (m, 1H), 4.61-4.54 (m, 1H), 4.51-4.32 (m, 3H), 4.27-4.18 (m, 2H), 4.07 (s, 3H), 3.93-3.67 (m, 4H), 3.44-3.38 (m, 2H), 3.29-3.09 (m, 3H), 2.22-1.97 (m, 8H), 0.98-0.84 (m, 4H). ¹⁹F NMR (376 MHz, methanol-d₄, ppm): δ −111.8 (1F), −150.6 (1F).

### Example 59 Synthesis of Compound 190 and 211

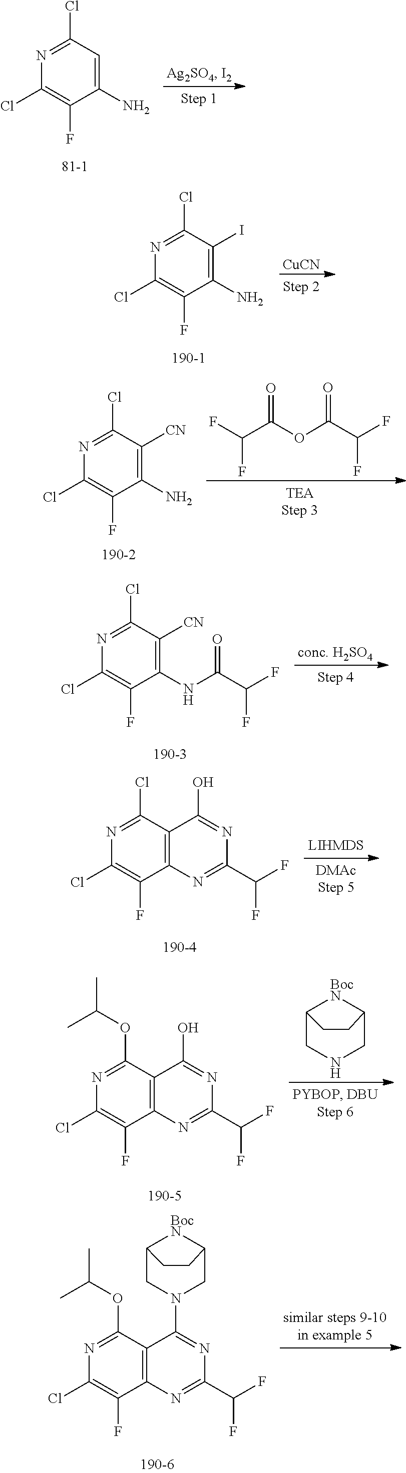

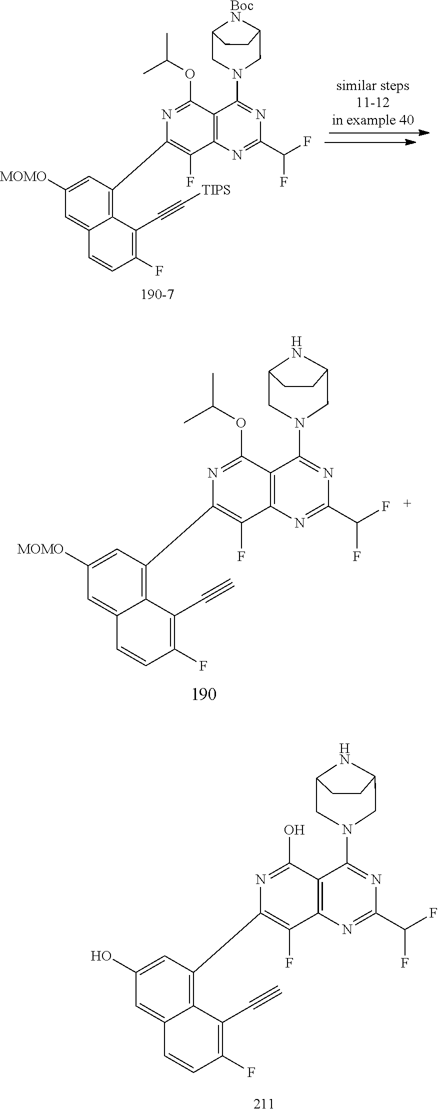

Step 1: To a mixture of 81-1 (5.43 g, 30.0 mmol) in ethanol (100 mL) was added silver sulfate (9.35 g, 30.0 mmol) and iodine (8.4 g, 33.0 mmol). The reaction mixture was stirred at room temperature for 2 hours. The mixture was filtered and washed with ethanol. The filtration was concentrated to give the residue, which was slurried with petroleum ether to afford 190-1.

Step 2: To a solution of 190-1 (4.4 g, 14.4 mmol) in N,N-dimethylformamide (50 mL) was added copper(I) cyanide (1.81 g, 20.2 mmol). The reaction mixture was stirred at 100° C. for 16 hours. The mixture was diluted with ethyl acetate and filtered. The filtration was washed with water and brine. The organic layer was dried over sodium sulfate, filtered and concentrated to give 190-2.

Step 3: To a solution of 190-2 (3.65 g, from step 2) and triethylamine (2.65 g, 26.2 mmol) in dichloromethane (27 mL) was added 2,2-difluoroacetic anhydride (3.42 g, 19.7 mmol) at 0° C. over 2 minutes. The resulting mixture was stirred at room temperature for 1 hour under N₂ atmosphere. The mixture was diluted with dichloromethane, washed with water and brine, dried over sodium sulfate, filtered and concentrated. The residue was purified by column chromatography on silica gel (petroleum ether to petroleum ether/ethyl acetate=4/1) to give 190-3.

Step 4: The mixture of 190-3 (2.4 g, 8.5 mmol) and concentrated sulfuric acid (13.8 mL, 253.9 mmol) was stirred at 60° C. for 1 hour under N₂ atmosphere. After cooling to room temperature, the mixture was added to ice-water and stirred for 30 minutes. The reaction mixture was extracted with ethyl acetate. The combined organic layers were washed with brine, dried over sodium sulfate, filtered and concentrated to afford 190-4.

Step 5: To a mixture of propan-2-ol (541 mg, 9 mmol) in dimethylacetamide (9 mL) was added lithium bis(trimethylsilyl)amide (9 mL, 9 mmol, 1 M in tetrahydrofuran) at 0° C., then stirred at 0° C. for 20 minutes. A solution of 190-4 (852 mg, 3 mmol) in dimethylacetamide (9 mL) was added to the mixture at 0° C. Then the reaction was stirred at 30° C. for 2 hours. The reaction mixture was diluted with water, adjusted to pH-4 with 2N hydrochloric acid and extracted with ethyl acetate. The combined organic layers were washed with brine, dried over sodium sulfate, filtered and concentrated to afford 190-5, which was used for next step directly.

Step 6: To a mixture of 190-5 (crude, from step 5), tert-butyl (1R,5S)-3,8-diazabicyclo[3.2.1]octane-8-carboxylate (1.3 g, 6.0 mmol), benzotriazole-1-yl-oxytripyrrolidinophosphonium hexafluorophosphate (3.2 g, 6.0 mmol) in N,N-dimethylformamide (20 mL) was added 1,8-diazabicyclo[5.4.0]undec-7-ene (912 mg, 6.0 mmol) at 0° C. under N₂ atmosphere. The resulting mixture was stirred at 40° C. for 3 hours and then diluted with ethyl acetate, washed with water and brine, dried over sodium sulfate, filtered and concentrated. The residue was purified by column chromatography on silica gel (dichloromethane to dichloromethane/ethyl acetate=10/1) to give 190-6.

Compound 190-7 was prepared from 190-6 and 128-10A following the procedure for the synthesis of compound 60-10 in example 5.

Compound 190 and 211 was prepared from 190-7 following the procedure for the synthesis of compound 128 in example 40 as a 2 eq of TFA salt, respectively.

190: LCMS (ESI, m/z): [M+H]⁺=552.3; ¹H NMR (400 MHz, methanol-d₄, ppm): δ 7.87-7.83 (m, 1H), 7.34-7.29 (m, 2H), 7.22-7.21 (m, 1H), 6.63 (t, J=54.4 Hz, 1H), 5.59-5.49 (m, 1H), 4.68-4.49 (m, 2H), 4.26-4.14 (m, 2H), 3.95-3.85 (m, 2H), 3.37 (s, 1H), 2.13-1.85 (m, 4H), 1.42 (d, J=6.0 Hz, 6H). ¹⁹F NMR (376 MHz, methanol-d₄, ppm): δ −111.81 (1F), −121.34 (2F), −149.12 (1F).

211: LCMS (ESI, m/z): [M+H]⁺=510.2; ¹H NMR (400 MHz, methanol-d₄, ppm): δ 7.91-7.86 (m, 1H), 7.41-7.30 (m, 3H), 6.60 (t, J=54.4 Hz, 1H), 4.59-4.51 (m, 1H), 4.48-4.44 (m, 1H), 4.25-4.15 (m, 2H), 3.72-3.63 (m, 3H), 2.28-2.18 (m, 2H), 2.11-2.03 (m, 2H). ¹⁹F NMR (376 MHz, methanol-d₄, ppm): δ −110.71 (1F), −121.80 (2F), −160.89 (1F).

### Example 60 Synthesis of Compound 198

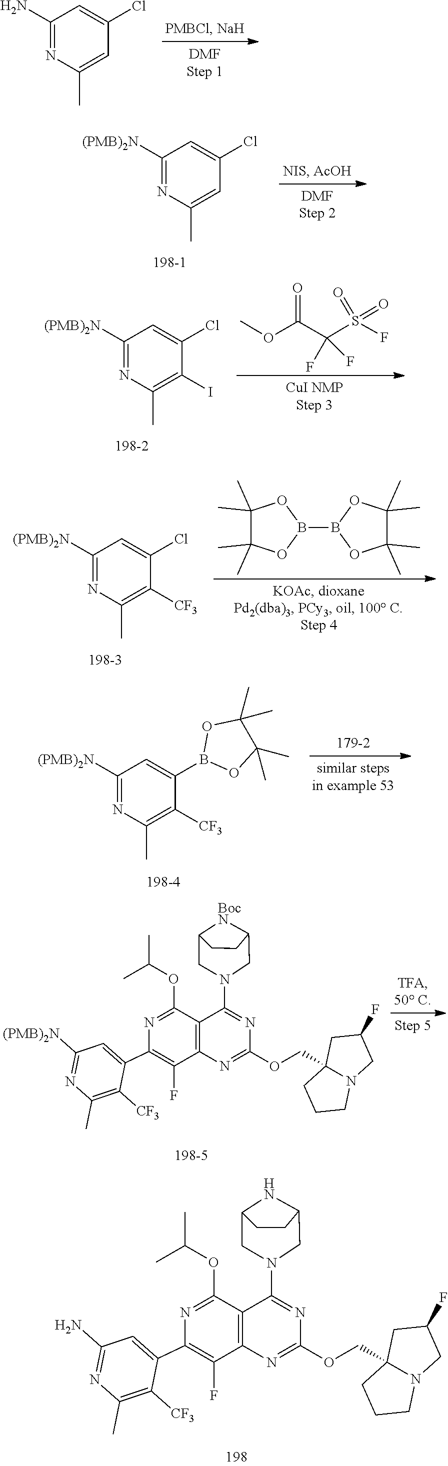

Step 1: A solution of 4-chloro-6-methylpyridin-2-amine (2 g, 14.026 mmol) in N, N-dimethylformamide (20 mL) was added sodium hydrogen (2.1 g, 53.300 mmol, 60% in mineral oil) at 0° C. under nitrogen atmosphere. After stirring for 30 minutes, 1-(chloromethyl)-4-methoxybenzene (4.202 mL, 30.858 mmol) was added into the solution. The resulting mixture was stirred at room temperature for 2 hours. The reaction was quenched with water and extracted with ethyl acetate. The combined organic layers were concentrated and purified by silica gel chromatography (petroleum ether to petroleum ether/ethyl acetate=6/1) to afford 198-1.

Step 2: To a mixture of 198-1 (500 mg, 1.306 mmol) and acetic acid (0.748 mL, 13.059 mmol) in N, N-dimethylformamide (7 mL) was added N-iodosuccinimide (271.1 mg, 1.567 mmol) at room temperature, stirred at room temperature for 2 hours. The resulting mixture was diluted with water and extracted with ethyl acetate. The organic layer was washed with water and concentrated. The residue was purified by silica gel chromatography (petroleum ether to petroleum ether/ethyl acetate=10/1) to afford 198-2.

Step 3: 198-2 (2.55 g, 5.012 mmol), methyl 2,2-difluoro-2-(fluorosulfonyl)acetate (1.914 mL, 15.036 mmol) and cuprous iodide (0.510 mL, 15.036 mmol) in 1-methyl-2-pyrrolidinone (50 mL) was stirred at 110° C. for 5 hours under nitrogen atmosphere. The resulting mixture was diluted with water and extracted with ethyl acetate. The organic layer was washed with water and concentrated. The residue was purified by silica gel chromatography (petroleum ether to petroleum ether/ethyl acetate=10/1) to afford 198-3.

Step 4: A solution of 198-3 (1.9 g, 4.214 mmol), potassium acetate (1.2 g, 12.642 mmol), 4,4,5,5-tetramethyl-2-(tetramethyl-1,3,2-dioxaborolan-2-yl)-1,3,2-dioxaborolane (2.14 g, 8.428 mmol), tricyclohexyl phosphine (0.2 g, 0.843 mmol) and tris(dibenzylideneacetone) dipalladium (0.4 g, 0.421 mmol) in dioxane (40 mL) was heated at 100° C. for 3 hours. The resulting mixture was diluted with water and extracted with ethyl acetate. The organic layer was washed with water and concentrated. The residue was purified by silica gel chromatography (petroleum ether to petroleum ether/ethyl acetate=10/1) to afford 198-4.

Compound 198-5 was prepared from 198-4 and 179-2 following the procedure for the synthesis of compound 155-8 in example 53.

Step 5: A solution of 198-5 (200 mg, 0.202 mmol) in trifluoroacetic acid (10 mL) was stirred for 3 hours at 50° C. under N₂ atmosphere. The mixture was concentrated and purified by prep-HPLC (acetonitrile/0.05% TFA in water: 5%˜95%) to give 198 as a 4 eq of TFA salt. LCMS (ESI, m/z): [M+H]⁺=649.4; ¹H NMR (400 MHz, methanol-d₄, ppm): δ 6.72 (s, 1H), 5.67-5.46 (m, 2H), 4.69-4.63 (m, 2H), 4.59-4.39 (m, 2H), 4.23-4.13 (m, 2H), 4.11-3.81 (m, 5H), 3.51-3.40 (m, 1H), 2.83-2.51 (m, 5H), 2.48-2.29 (m, 3H), 2.26-2.12 (m, 1H), 2.11-1.88 (m, 4H), 1.43-1.39 (m, 6H). ¹⁹F NMR (376 MHz, methanol-d₄, ppm): δ −55.34 (3F), −152.17 (1F), −174.06 (1F).

### Example 61 Synthesis of Compound 199

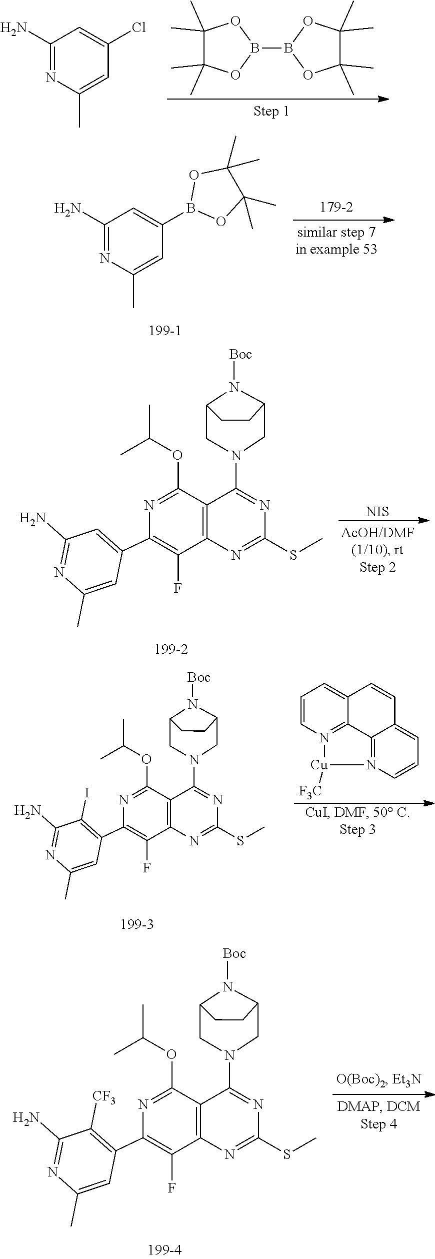

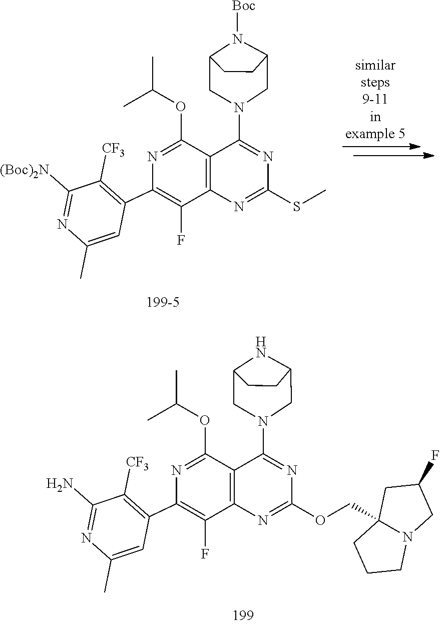

Step 1: A solution of 4-chloro-6-methylpyridin-2-amine (1400 mg, 9.818 mmol), potassium acetate (2890.72 mg, 29.455 mmol), 4,4,5,5-tetramethyl-2-(tetramethyl-1,3,2-dioxaborolan-2-yl)-1,3,2-dioxaborolane (5.088 mL, 19.637 mmol), tricyclohexyl phosphine (550.67 mg, 1.964 mmol) and tris(dibenzylideneacetone) dipalladium (899.09 mg, 0.982 mmol) in dioxane (20 mL) was heated at 120° C. in a microwave for 30 minutes. The solution was filtered and concentrated. Glyme (50 mL) was added and the resulting solid was filtered and rinsed with dichloromethane (30 mL). The combined filtrate was concentrated to afford 199-1 which was used directly.

Compound 199-2 was prepared from 199-1 and 179-2 following the procedure for the synthesis of compound 155-7 in example 53.

Step 2: A mixture of 199-2 (950 mg, 1.67 mmol) and acetic acid (1 mL) in N, N-dimethylformamide (10 mL) was added N-iodosuccinimide (450 mg, 2 mmol) at 0° C. under nitrogen atmosphere, then stirred at room temperature for 5 hours. The resulting mixture was quenched with water and adjusted to pH to 8 with saturated aqueous sodium bicarbonate, extracted with ethyl acetate. The combined organic layers were concentrated and purified by silica gel chromatography (petroleum ether to petroleum ether/ethyl acetate=3/1) to afford S199-3.

Step 3: 199-3 (660 mg, 0.948 mmol), (1,10-phenanthroline)(trifluoromethyl)copper(I) (2968.10 mg, 9.483 mmol) and cuprous iodide (180.6 mg, 0.948 mmol) in N, N-dimethylformamide (10 mL) was stirred at 50° C. for 5 hours under nitrogen atmosphere. The resulting mixture was diluted with ethyl acetate and filtered, the filtrate was diluted with water and the organic layers was separated and washed with water, concentrated and purified by silica gel chromatography (petroleum ether to petroleum ether/ethyl acetate=3/1) to afford 199-4.

Step 4: A mixture of 199-4 (280 mg, 0.439 mmol), di-tert-butyl dicarbonate (0.207 mL, 0.966 mmol), triethylamine (0.214 mL, 1.537 mmol) and 4-dimethylaminopyridine (5.4 mg, 0.044 mmol) in dichloromethane (10 mL) was stirred at room temperature for 4 hours. The resulting mixture was quenched with water and extracted with dichloromethane. The combined organic layers were concentrated and purified by silica gel chromatography (petroleum ether to petroleum ether/ethyl acetate=9/1) to afford 199-5.

Compound 199 was prepared from 199-5 following the procedure for the synthesis of compound 60 in example 5 as a 5 eq of TFA salt. LCMS (ESI, m/z): [M+H]⁺=649.3; ¹H NMR (400 MHz, methanol-d₄, ppm): δ 6.59 (s, 1H), 5.73-5.43 (m, 2H), 4.68-4.64 (m, 2H), 4.59-4.38 (m, 2H), 4.26-4.12 (m, 2H), 4.09-3.75 (m, 5H), 3.55-3.40 (m, 1H), 2.83-2.50 (m, 5H), 2.48-2.25 (m, 3H), 2.25-2.10 (m, 1H), 2.08-1.88 (m, 4H), 1.47-1.36 (m, 6H). ¹⁹F NMR (376 MHz, methanol-d₄, ppm): δ −55.23 (3F), −152.42 (1F), −174.09 (1F).

### Example 62 Synthesis of Compound 202

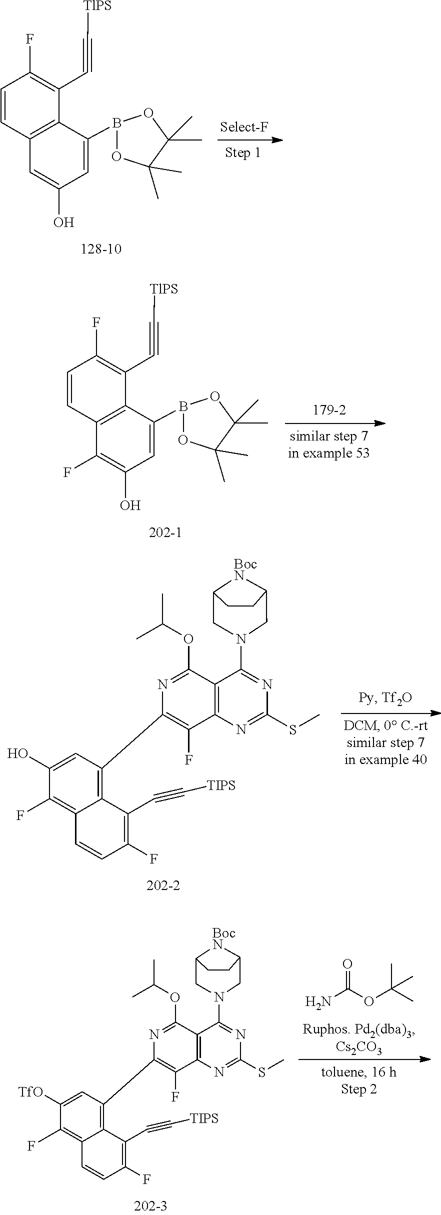

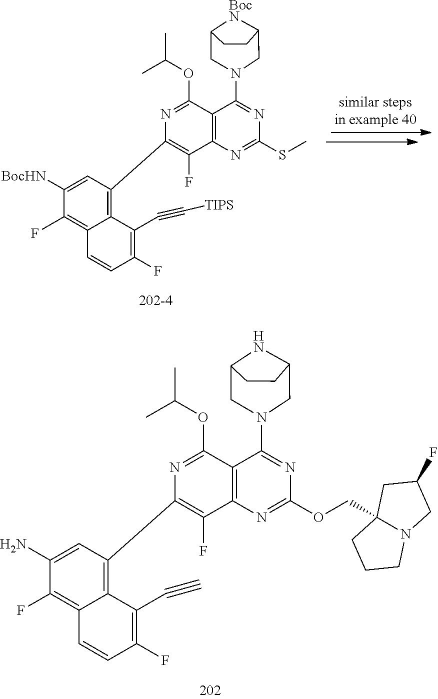

Step 1: To a mixture of 128-10 (2.1 g, 4.3 mmol) in acetonitrile (45 mL) was added selectfluor (1.82 g, 5.2 mmol) at room temperature. The mixture was stirred at room temperature for 3 hours. The mixture was diluted with ethyl acetate, washed with water and brine, dried over sodium sulfate, filtered and concentrated. The residue was purified by column chromatography on silica gel (petroleum ether to petroleum ether/ethyl acetate=0-10%˜20%) to afford 202-1.

Compound 202-2 was prepared from 202-1 and 179-2 following the procedure for the synthesis of compound 155-7 in example 53.

Compound 202-3 was prepared from 202-2 following the procedure for the synthesis of compound 128-7 in example 40.

Step 2: The mixture of 202-3 (300 mg, 0.31 mmol), tert-butyl carbamate (111 mg, 0.94 mmol), cesium carbonate (307 mg, 0.94 mmol), tris(dibenzylideneacetone)dipalladium (28.8 mg, 0.03 mmol), and 2-dicyclohexylphosphino-2′,6′-diisopropoxy-1,1′-biphenyl (29.3 mg, 0.06 mmol) in toluene (15 mL) was stirred at 100° C. for 16 hours under N₂ atmosphere. After cooling to room temperature, the reaction solution was diluted with ethyl acetate, washed water, brine, dried over sodium sulfate, filtered and concentrated. The residue was purified by column chromatography on silica gel (petroleum ether to petroleum ether/ethyl acetate=4/1) to give 202-4.

Compound 202 was prepared from 202-4 following the procedure for the synthesis of compound 128 in example 40 as a 4 eq of TFA salt. LCMS (ESI, m/z): [M+H]⁺=676.4; ¹H NMR (400 MHz, methanol-d₄, ppm): δ 8.06-8.02 (m, 1H), 7.39-7.34 (m, 1H), 7.28-7.26 (m, 1H), 5.65-5.48 (m, 2H), 4.73-4.64 (m, 2H), 4.59-4.44 (m, 2H), 4.25-4.14 (m, 2H), 4.10-3.80 (m, 5H), 3.50-3.38 (m, 2H), 2.78-2.52 (m, 2H), 2.47-2.27 (m, 3H), 2.25-1.92 (m, 5H), 1.43-1.38 (m, 6H). ¹⁹F NMR (376 MHz, methanol-d₄, ppm): δ −112.45 (1F), −147.74 (1F), −151.02 (1F), −174.10 (1F).

### Example 63 Synthesis of Compound 203

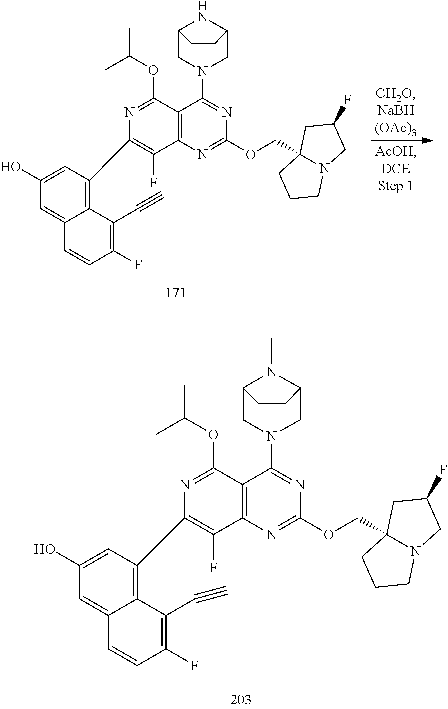 [SMILES: C#Cc1c(F)ccc2cc(O)cc(-c3nc(OC(C)C)c4c(N5CC6CCC(C5)N6)nc(OC[C@@]56CCCN5C[C@H](F)C6)nc4c3F)c12 ; C#Cc1c(F)ccc2cc(O)cc(-c3nc(OC(C)C)c4c(N5CC6CCC(C5)N6C)nc(OC[C@@]56CCCN5C[C@H](F)C6)nc4c3F)c12]

Step 1: To a solution of 171 (90 mg, 0.137 mmol) in 1,2-dichloroethane (5 mL) was added acetic acid (16 mg, 0.273 mmol) and 37% formaldehyde solution (111 mg, 1.3 mmol), then stirred at room temperature for 30 mins. To above mixture was added sodium triacetoxyborohydride (212 mg, 0.683 mmol), then stirred at room temperature for 30 mins. The mixture was diluted with aqueous sodium bicarbonate solution, extracted with dichloromethane. The combined organic layers were washed with brine, dried over anhydrous sodium sulfate, filtered and concentrated. The residue was purified by prep-HPLC (acetonitrile/0.05% TFA in water: 10%˜95%) to give 203 as 3.0 TFA salt. LCMS (ESI, m/z): [M+H]⁺=673.4; ¹H NMR (400 MHz, methanol-d₄, ppm): δ 7.89-7.81 (m, 1H), 7.37-7.26 (m, 2H), 7.24-7.12 (s, 1H), 5.67-5.47 (m, 2H), 4.76-4.49 (m, 4H), 4.14-3.81 (m, 7H), 3.53-3.42 (m, 1H), 3.40-3.34 (m, 1H), 2.89 (s, 3H), 2.78-2.52 (m, 2H), 2.48-2.00 (m, 8H), 1.46-1.36 (m, 6H). ¹⁹F NMR (376 MHz, methanol-d₄, ppm): δ −111.8 (1F) −151.4 (1F), −174.2 (1F).

### Example 64 Synthesis of Compound 205

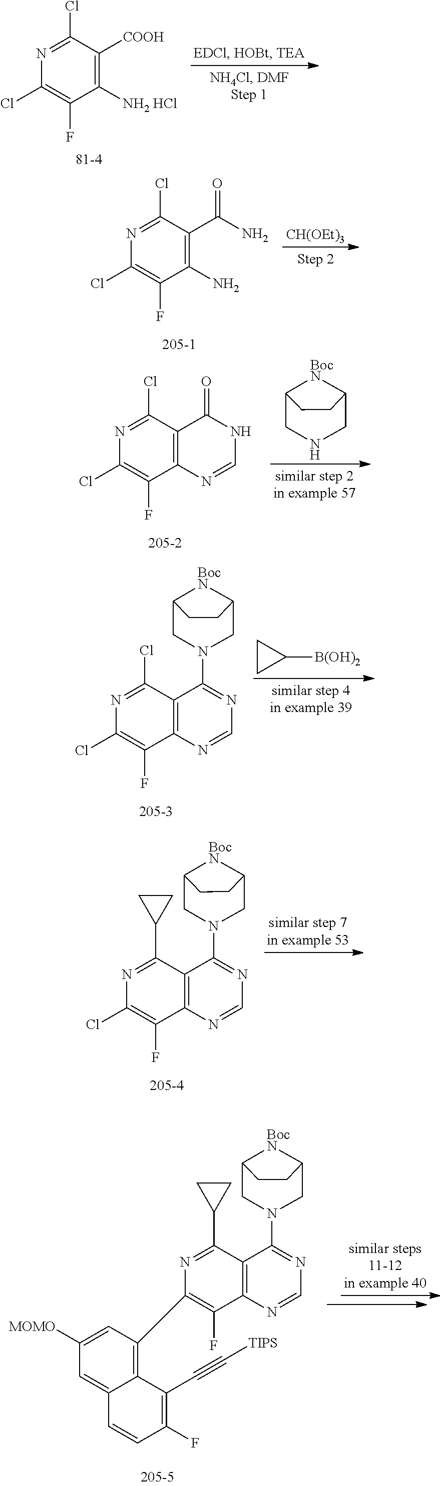

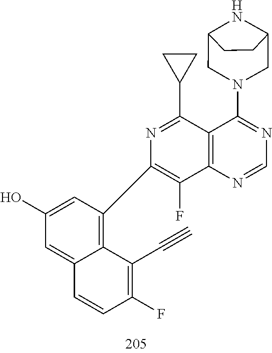 [SMILES: C#Cc1c(F)ccc2cc(O)cc(-c3nc(C4CC4)c4c(N5CC6CCC(C5)N6)ncnc4c3F)c12]

Step 1: To a solution of 81-4 (20 g, 76.77 mmol, 1.0 equiv.) in DMF (300 mL) were added NH₄Cl (12.3 g, 231.38 mmol, 3.0 equiv.), HOBt (15.6 g, 115.45 mmol, 1.5 equiv.), TEA (53.2 mL, 383.85 mmol, 5.0 equiv.) and EDCI (22.1 g, 115.28 mmol, 1.5 equiv.), and the reaction was stirred at 60° C. for 3 hours. The reaction was diluted with ethyl acetate and water. The organic layer was separated, washed with water and brine. Then the organic layer was dried over anhydrous sodium sulfate and concentrated in vacuo to give 205-1.

Step 2: To a solution of 205-1 (11 g, 49.10 mmol, 1.0 equiv.) in triethyl orthoformate (275 mL, 25V) were stirred at 180° C. for 72 hours. The layer was filtered and the solid was washed by DCM to afford 205-2.

Compound 205-3 was prepared from 205-2 following the procedure for the synthesis of compound 179-2 in example 57.

Compound 205-4 was prepared from 205-3 following the procedure for the synthesis of compound 122-8 in example 39.

Compound 205-5 was prepared from 205-4 and 128-10A following the procedure for the synthesis of compound 155-7 in example 53.

Compound 205 was prepared from 205-5 following the procedure for the synthesis of compound 128 in example 40 as 3.0 TFA salt. LCMS (ESI, m/z): [M+H]⁺=484.3; ¹H NMR (400 MHz, methanol-d₄, ppm): δ 8.70 (s, 1H), 7.89-7.82 (m, 1H), 7.36-7.28 (m, 2H), 7.23-7.10 (m, 1H), 4.84-4.79 (m, 1H), 4.51-3.82 (m, 5H), 3.22 (s, 1H), 2.42-2.28 (m, 1H), 2.22-1.61 (m, 4H), 1.42-0.92 (m, 4H). ¹⁹F NMR (376 MHz, methanol-d₄, ppm): δ −111.95 (1F), −145.66 (1F).

### Example 65 Synthesis of Compound 213

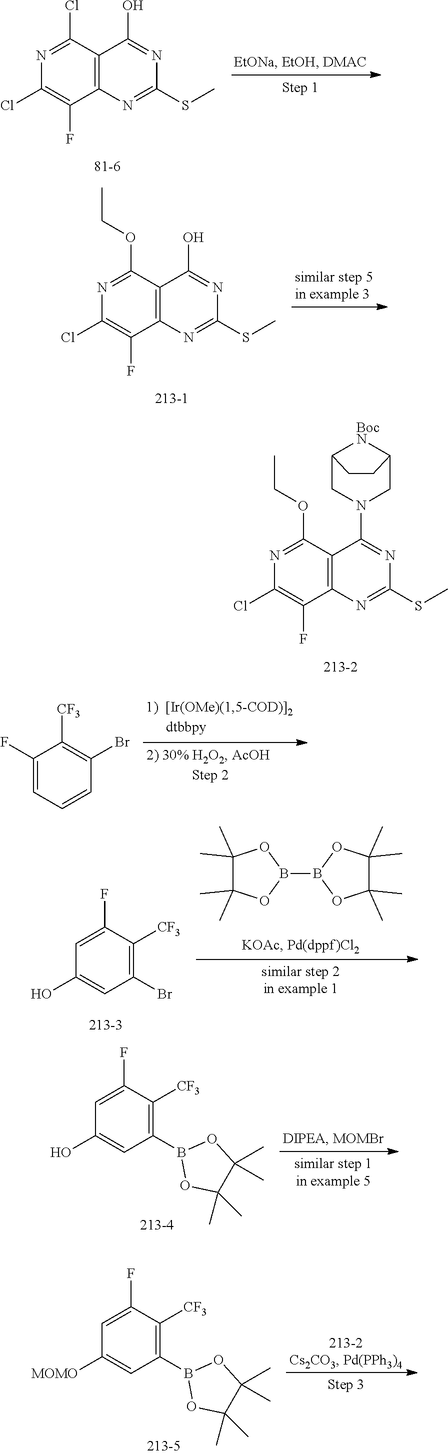

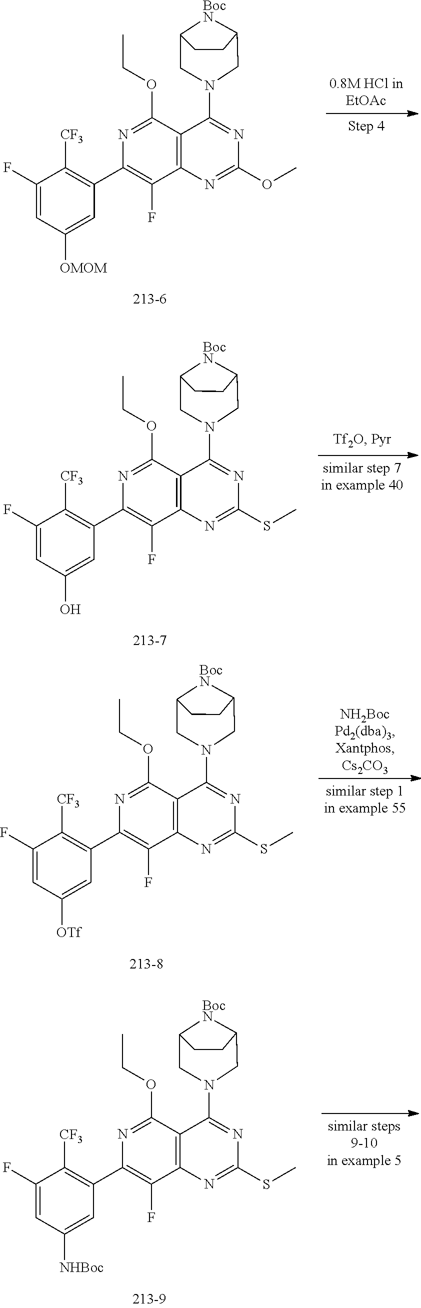

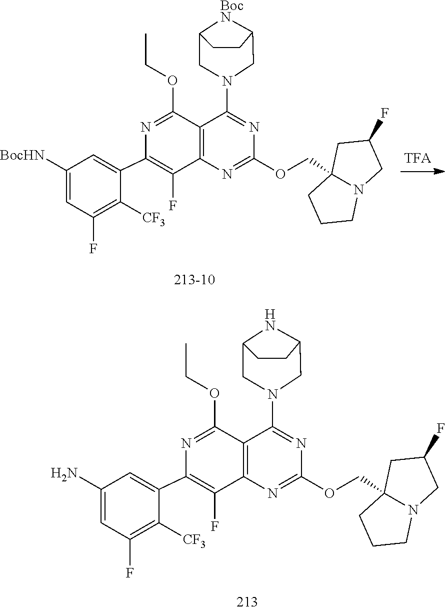 [SMILES: CCOc1nc(-c2cc(NC(=O)OC(C)(C)C)cc(F)c2C(F)(F)F)c(F)c2nc(OC[C@@]34CCCN3C[C@H](F)C4)nc(N3CC4CCC(C3)N4C(=O)OC(C)(C)C)c12 ; CCOc1nc(-c2cc(N)cc(F)c2C(F)(F)F)c(F)c2nc(OC[C@@]34CCCN3C[C@H](F)C4)nc(N3CC4CCC(C3)N4)c12]

Step 1: To a mixture of 81-6 (700 mg, 2.5 mmol) in dimethylacetamide (15 mL) and ethanol (2.5 mL) was added sodium ethoxide (510 mg, 7.5 mmol) at room temperature, then stirred for 2 hours. The mixture was diluted with water, adjusted to pH-3 with 2N hydrochloric acid. The suspension was filtered and the filter cake was slurried to give 213-1.

Compound 213-2 was prepared from 213-1 following the procedure for the synthesis of compound 10-5 in example 3.

Step 2: The mixture of 1-bromo-3-fluoro-2-(trifluoromethyl)benzene (4.86 g, 20 mmol), 4,4,5,5-tetramethyl-1,3,2-dioxaborolane (5.16 g, 40 mmol), 4,4′-di-tert-butyl-2,2′-bipyridine (643 mg, 2.4 mmol) and bis(1,5-cyclooctadiene)dimethoxydiiridium (1.33 g, 2 mmol) in tetrahydrofuran (60 mL) was stirred at 60° C. for 2.5 hours under N₂ atmosphere. After the solvent was removed, the residue was dissolved in tetrahydrofuran/water (2/1, 150 mL). To above mixture was added acetic acid (4.8 g, 80 mmol) and 30% hydrogen peroxide (45.3 g, 400 mmol) at 10° C. The resulting mixture was stirred at room temperature for 1 hour under air atmosphere. The mixture was diluted with ethyl acetate, washed with water, saturated aqueous sodium sulfite and brine, dried over sodium sulfate, filtered and concentrated. The residue was purified by column chromatography on silica gel (petroleum ether to petroleum ether/ethyl acetate=4/1) to give 213-3.

Compound 213-4 was prepared from 213-3 following the procedure for the synthesis of compound 20-2 in example 1.

Compound 213-5 was prepared from 213-4 following the procedure for the synthesis of compound 60-1 in example 5.

Step 3: To a mixture of 213-2 (968 mg, 2 mmol), 213-5 (700 mg, 2 mmol), cesium carbonate (1.9 g, 6 mmol) and tetrakis(triphenylphosphine)palladium (231 mg, 0.2 mmol) in 1,4-dioxane (12 mL) and water (3 mL) was stirred at 130° C. for 80 minutes under N₂ atmosphere with microwave. The reaction was diluted with ethyl acetate, washed with water and brine, dried over sodium sulfate, filtered and concentrated. The residue was purified by column chromatography on silica gel (petroleum ether to petroleum ether/ethyl acetate=4/1) to give 213-6.

Step 4: The mixture of 213-6 (672 mg, 1 mmol) and 0.8M hydrochloric acid in ethyl acetate (12 mL, 10 mmol) in ethyl acetate (87.5 mL) was stirred at room temperature for 16 hours under N₂ atmosphere. The mixture was added to saturated aqueous sodium bicarbonate and separated. The Organic lay was washed with brine, dried over sodium sulfate, filtered and concentrated. The residue was purified by column chromatography on silica gel (petroleum ether to petroleum ether/ethyl acetate=4/1) to give 213-7.

Compound 213-8 was prepared from 213-7 following the procedure for the synthesis of compound 128-7 in example 40.

Compound 213-9 was prepared from 213-8 following the procedure for the synthesis of compound 170-1 in example 55.

Compound 213-10 was prepared from 213-9 following the procedure for the synthesis of compound 60-10 in example 5.

Step 4: A solution of 213-10 (170 mg, 0.20 mmol) in trifluoroacetic acid (1 mL) and dichloromethane (3 mL) was stirred at room temperature for 1 hour under N₂ atmosphere. The reaction mixture was concentrated and the residue was purified by prep-HPLC (acetonitrile/0.05% TFA in water: 5%˜95%) to give 213 as a 3 eq of TFA salt. LCMS (ESI, m/z): [M+H]⁺=638.4; ¹H NMR (400 MHz, methanol-d₄, ppm): δ 6.54 (d, J=14.0 Hz, 1H), 6.44 (d, J=1.6 Hz, 1H), 5.66-5.49 (m, 1H), 4.70-4.43 (m, 6H), 4.21-4.15 (m, 2H), 4.08-3.76 (m, 5H), 3.51-3.42 (m, 1H), 2.78-2.52 (m, 2H), 2.47-2.30 (m, 3H), 2.24-1.96 (m, 5H), 1.44 (t, J=7.2 Hz, 3H). ¹⁹F NMR (376 MHz, methanol-d₄, ppm): δ −55.79 (3F), −114.60 (1F), −151.94 (1F), −174.13 (1F).

### Example 66 Synthesis of Compound 178

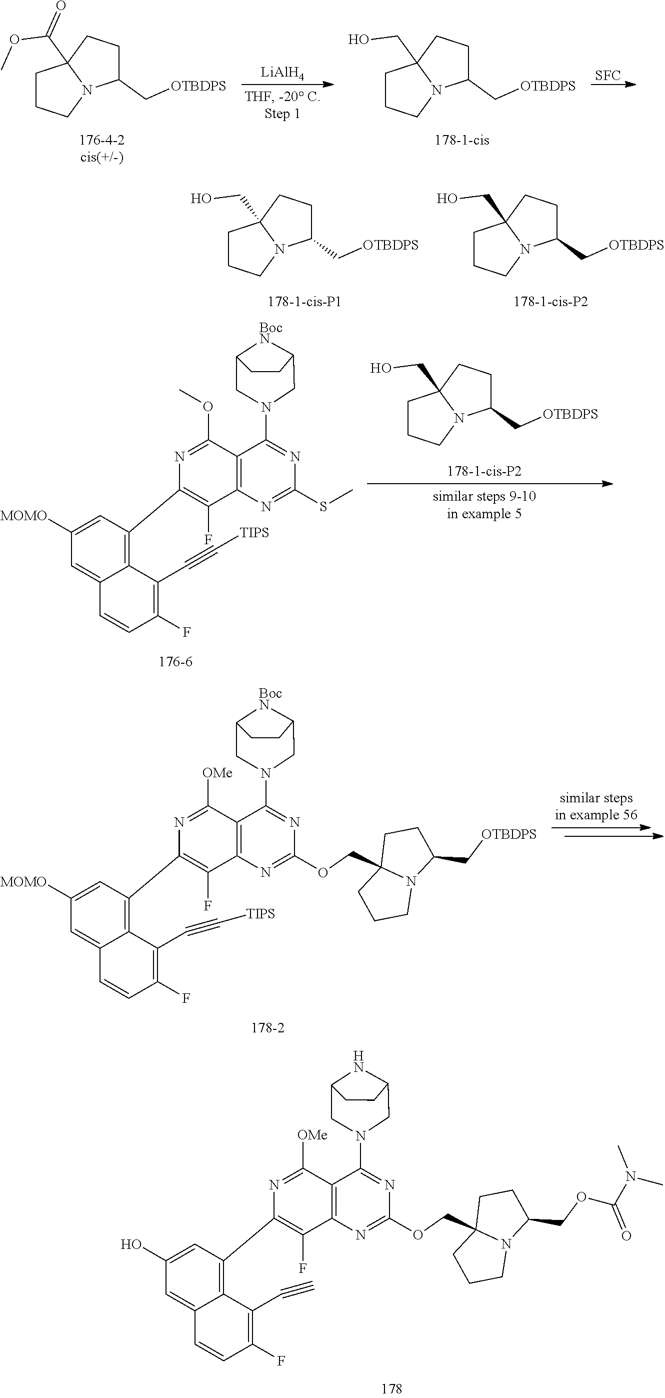

Step 1: To a solution of 176-4-2 (cis, +/−) (4.2 g, 9.596 mmol) in tetrahydrofuran (50 mL) was added lithium aluminium hydride (0.73 g, 19.193 mmol) at −20° C. under nitrogen atmosphere, then stirred for 1 hour. Then the mixture was quenched with water, 15% sodium hydroxide aqueous and water at 0° C. The mixture was diluted with tetrahydrofuran, dried over sodium sulfate, filtered and concentrated to give 178-1-cis (+/−).

178-1-cis (cis, +/−) (3.9 g, 8.911 mmol) was purified by preparative SFC [DAICELCHIRALCEL®OZ with MEOH (+0.1% 7.0 mol/L Ammonia in MEOH)/Supercritical C02] to give 178-1-cis-P1 and 178-1-cis-P2.

178-1-cis-P1: Chiral SFC analysis: >99.5% ee. Retention time 4.792 min on DAICELCHIRALCEL®OZ 100*3 mm 3 μm column (35° C.); mobile phase: methanol (0.1% DEA) in CO₂, 1800 psi, 1.5 mL/min.

178-1-cis-P2: Chiral SFC analysis: 95.04% ee. Retention time 5.030 min on DAICELCHIRALCEL®OZ 100*3 mm 3 μm column (35° C.); mobile phase: methanol (0.1% DEA) in CO₂, 1800 psi, 1.5 mL/min.

Compound 178-2 was prepared from 176-6 and 178-1-cis-P2 following the procedure for the synthesis of compound 60-10 in example 5.

Compound 178 was prepared from 178-2 following the procedure for the synthesis of compound 176 in example 56 as a 3 eq of TFA salt. LCMS (ESI, m/z): [M+H]⁺=714.4; ¹H NMR (400 MHz, methanol-d₄, ppm): δ 7.89-7.82 (m, 1H), 7.38-7.27 (m, 2H), 7.25-7.21 (m, 1H), 4.75-4.61 (m, 2H), 4.54-4.41 (m, 3H), 4.35-4.16 (m, 3H), 4.08-4.04 (m, 3H), 3.91-3.82 (m, 1H), 3.79-3.62 (m, 3H), 3.59-3.47 (m, 1H), 3.39-3.34 (m, 1H), 2.89-2.75 (m, 6H), 2.54-2.44 (m, 1H), 2.33-2.04 (m, 11H). ¹⁹F NMR (376 MHz, methanol-d₄, ppm): δ −111.74 (1F), −150.31 (1F).

### Example 67 Synthesis of Compound 185

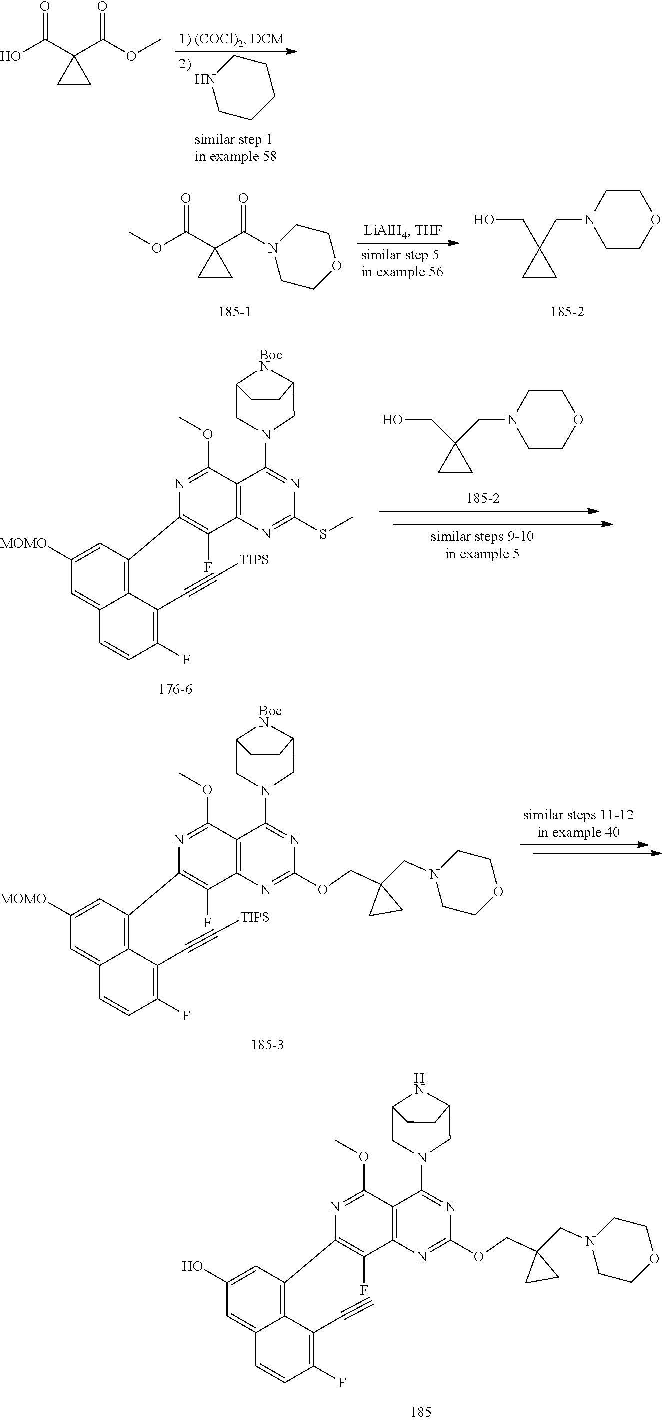

Compound 185-1 was prepared from 1-(methoxycarbonyl)cyclopropane-1-carboxylic acid following the procedure for the synthesis of compound 183-1 in example 58.

Compound 185-2 was prepared from 185-1 following the procedure for the synthesis of compound 176-5 in example 56.

Compound 185-3 was prepared from 176-6 and 185-2 following the procedure for the synthesis of compound 60-10 in example 5.

Compound 185 was prepared from 185-3 following the procedure for the synthesis of compound 128 in example 40 as a 3 eq of TFA salt. LCMS (ESI, m/z): [M+H]⁺=643.4; ¹H NMR (400 MHz, methanol-d₄, ppm): δ 7.89-7.82 (m, 1H), 7.38-7.29 (m, 2H), 7.22 (d, J=2.4 Hz, 1H), 4.56-4.34 (m, 4H), 4.29-4.17 (m, 2H), 4.14-3.96 (m, 5H), 3.93-3.64 (m, 6H), 3.43-3.32 (m, 3H), 3.29-3.08 (m, 2H), 2.22-2.06 (m, 4H), 1.03-0.82 (m, 4H). ¹⁹F NMR (376 MHz, methanol-d₄, ppm): δ −111.80 (1F), −150.10 (1F).

### Example 68 Synthesis of Compound 204

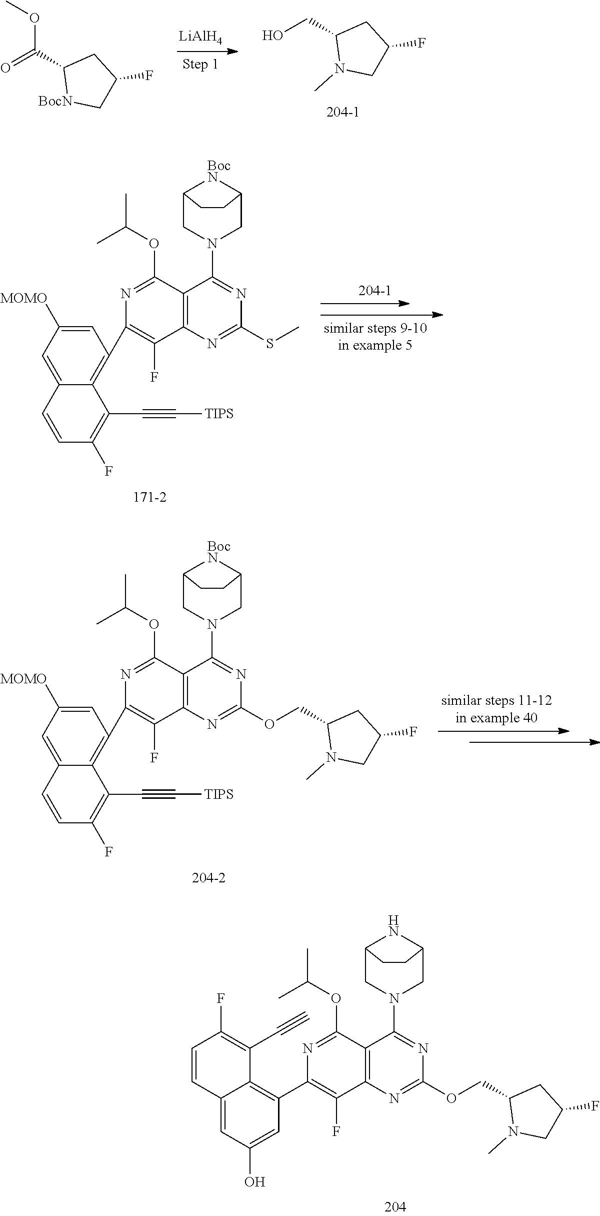

Step 1: To a solution of 1-tert-butyl 2-methyl (2S,4S)-4-fluoropyrrolidine-1,2-dicarboxylate (400 mg, 1.618 mmol) in tetrahydrofuran (4 mL) was added lithium aluminum hydride (184.2 mg, 4.854 mmol) in batches at 0° C. and the reaction was stirred at this temperature for 1 hour. Then the reaction was stirred at 65° C. for 30 mins. The reaction was quenched with sodium sulfate decahydrate. The suspension was filtered and the filtrate was concentrated to give the desired product 204-1.

Compound 204-2 was prepared from 171-2 and 204-1 following the procedure for the synthesis of compound 60-10 in example 5.

Compound 204 was prepared from 204-2 following the procedure for the synthesis of compound 128 in example 40 as a 6 eq of TFA salt. LCMS (ESI, m/z): [M+H]⁺=633.4; ¹H NMR (400 MHz, methanol-d₄, ppm): δ 7.87-7.83 (m, 1H), 7.34-7.30 (m, 2H), 7.19 (dd, J=4.0, 2.4 Hz, 1H), 5.56-5.40 (m, 2H), 5.00-4.96 (m, 1H), 4.69-4.64 (m, 1H), 4.59-4.55 (m, 1H), 4.51-4.47 (m, 1H), 4.22-4.19 (m, 2H), 4.10-4.07 (m, 1H), 4.01-3.94 (m, 1H), 3.90-3.84 (m, 2H), 3.55-3.43 (m, 1H), 3.39-3.35 (m, 1H), 3.18 (s, 3H), 2.89-2.77 (m, 1H), 2.43-2.32 (m, 1H), 2.06-1.96 (m, 4H), 1.43-1.40 (m, 6H). ¹⁹F NMR (376 MHz, methanol-d₄, ppm): δ −111.85 (1F), −151.39 (1F), −173.74 (1F).

### Example 69 Synthesis of Compound 217

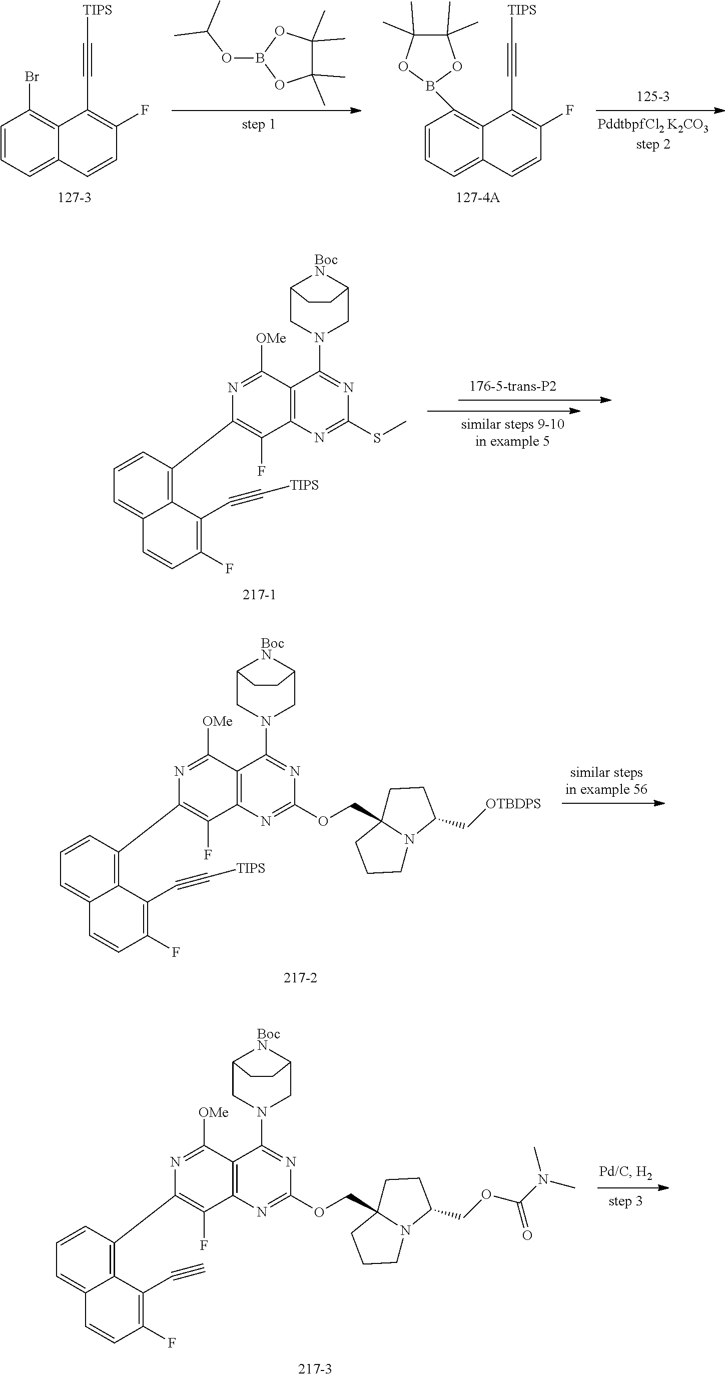

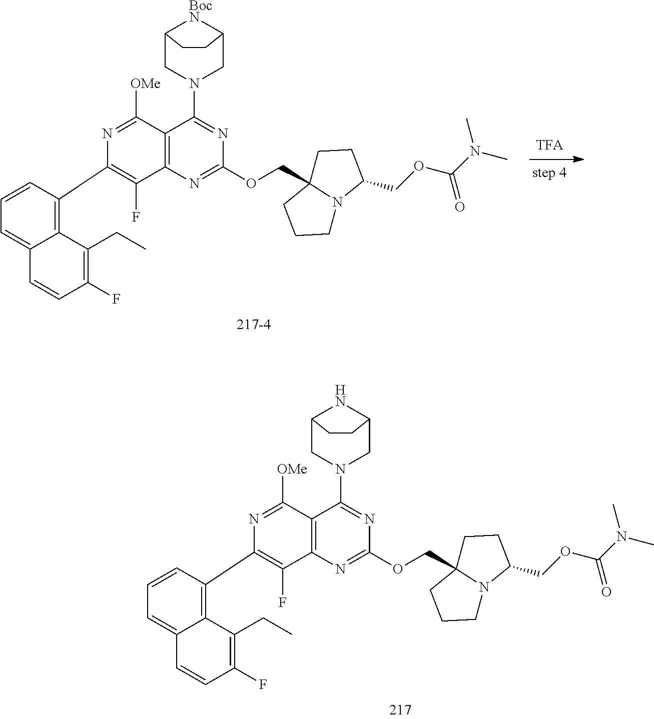 [SMILES: CCc1c(F)ccc2cccc(-c3nc(OC)c4c(N5CC6CCC(C5)N6C(=O)OC(C)(C)C)nc(OC[C@]56CCCN5[C@@H](COC(=O)N(C)C)CC6)nc4c3F)c12 ; CCc1c(F)ccc2cccc(-c3nc(OC)c4c(N5CC6CCC(C5)N6)nc(OC[C@]56CCCN5[C@@H](COC(=O)N(C)C)CC6)nc4c3F)c12]

Step 1: To a solution of 127-3 (6.29 g, 15.5 mmol) in tetrahydrofuran (58 mL) was added dropwise n-butyllithium (6.83 mL, 17.1 mmol, 2.5M in tetrahydrofuran) at −60° C. over 10 minutes under N₂ atmosphere. The mixture was stirred for 15 minutes and a solution of 2-isopropoxy-4,4,5,5-tetramethyl-1,3,2-dioxaborolane (4.35 g, 23.3 mmol) in tetrahydrofuran (5 mL) was added thereto over 10 minutes. The resulting mixture was stirred for another 20 minutes and quenched with water, extracted with ethyl acetate, the organics were washed with brine, dried over sodium sulfate, filtered and concentrated. The residue was purified by column chromatography on silica gel (petroleum ether) to give 127-4A.

Step 2: A mixture of 125-3 (1410 mg, 3.00 mmol), 127-4A (1561 mg, 3.45 mmol), potassium carbonate (829.3 mg, 6.0 mmol) and 1,1′-bis (di-t-butylphosphino)ferrocene palladium dichloride (195.5 mg, 0.3 mmol) in 1,4-dioxane/water (5/1, 180 mL) was stirred at 110° C. for 2 hours under nitrogen atmosphere. The reaction mixture was concentrated and diluted with brine, extracted with ethyl acetate. The combined organic layers were washed with brine, dried over anhydrous sodium sulfate, filtered and concentrated. The residue was purified by column chromatography on silica gel (petroleum ether to petroleum ether/ethyl acetate=5/1) to give 217-1.

Compound 217-2 was prepared from 217-1 and 176-5-trans-P2 following the procedure for the synthesis of compound 60-10 in example 5.

Compound 217-3 was prepared from 217-3 following the procedure for the synthesis of compound 176-9 in example 56.

Step 3: To a solution of 217-3 (260 mg, 0.326 mmol) in ethyl acetate (4 mL) was added 10% Pd/C (56 mg, 20% w). The mixture was stirred at room temperature for 16 hours under hydrogen atmosphere. The reaction mixture was filtered through a celite pad and concentrated to give 217-4.

Step 4: To a solution of 217-4 (240 mg, 0.299 mmol) in dichloromethane (5 mL) was added trifluoroacetic acid (2.0 mL) at 0° C., then stirred at room temperature for 2 hours. The reaction mixture was concentrated and purified by prep-HPLC (acetonitrile/0.05% TFA in water: 5%˜95%) to give 217 as a 2 eq of TFA salt. LCMS (ESI, m/z): [M+H]⁺=702.5; ¹H NMR (400 MHz, methanol-d₄, ppm): δ 8.07-7.99 (s, 1H), 7.95-7.87 (m, 1H), 7.58-7.50 (m, 1H), 7.49-7.43 (m, 1H), 7.42-7.30 (m, 1H), 4.76-5.60 (m, 2H), 4.57-4.41 (m, 3H), 4.40-4.18 (m, 4H), 4.05 (s, 3H), 3.82-3.72 (m, 2H), 3.65-3.57 (m, 1H), 3.47-3.37 (m, 1H), 2.99-2.79 (m, 6H), 2.67-2.58 (m, 1H), 2.48-2.33 (m, 3H), 2.27-2.04 (m, 10H), 0.91-0.81 (m, 3H). ¹⁹F NMR (376 MHz, methanol-d₄, ppm): δ −111.60 (1F), −149.35 (1F).

### Example 70 Synthesis of Compound 218

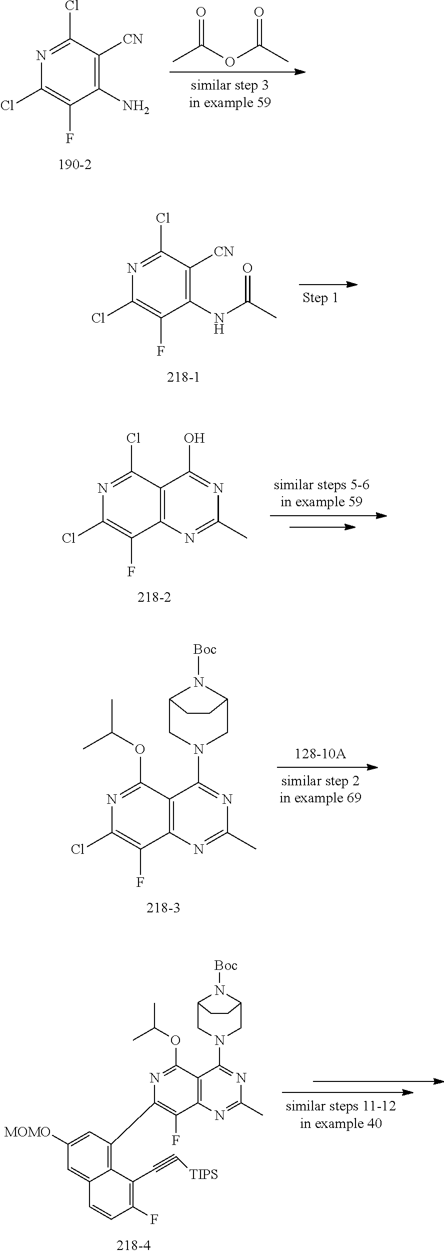

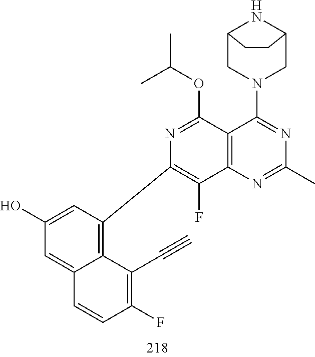 [SMILES: C#Cc1c(F)ccc2cc(O)cc(-c3nc(OC(C)C)c4c(N5CC6CCC(C5)N6)nc(C)nc4c3F)c12]

Compound 218-1 was prepared from 190-2 following the procedure for the synthesis of compound 190-3 in example 59.

Step 1: The mixture of 218-1 (1.30 g, 5.24 mmol) in a solution of 4.0 mol hydrochloric acid in ethyl acetate (40 mL) was stirred at 40° C. for 16 hours under N₂ atmosphere. After cooling to room temperature, the mixture was concentrated. The residue was diluted with ethyl acetate, washed with saturated aqueous sodium bicarbonate, brine, dried over sodium sulfate, filtered and concentrated to afford 218-2.

Compound 218-3 was prepared from 218-2 following the procedure for the synthesis of compound 190-6 in example 59.

Compound 218-4 was prepared from 218-3 and 128-10A following the procedure for the synthesis of compound 217-1 in example 69.

Compound 218 was prepared from 218-4 following the procedure for the synthesis of compound 128 in example 40 as a 3 eq of TFA salt. LCMS (ESI, m/z): [M+H]⁺=516.0; ¹H NMR (400 MHz, methanol-d₄, ppm): δ 7.89-7.85 (m, 1H), 7.35-7.31 (m, 2H), 7.22 (d, J=2.4 Hz, 1H), 5.60-5.51 (m, 1H), 4.71-4.58 (m, 2H), 4.28-4.17 (m, 2H), 3.98-3.86 (m, 2H), 3.44 (s, 1H), 2.68 (s, 3H), 2.15-2.02 (m, 2H), 1.99-1.89 (m, 2H), 1.55-1.35 (m, 6H). ¹⁹F NMR (376 MHz, methanol-d₄, ppm): δ −111.65 (1F), −150.63 (1F).

### Example 71 Synthesis of Compound 219

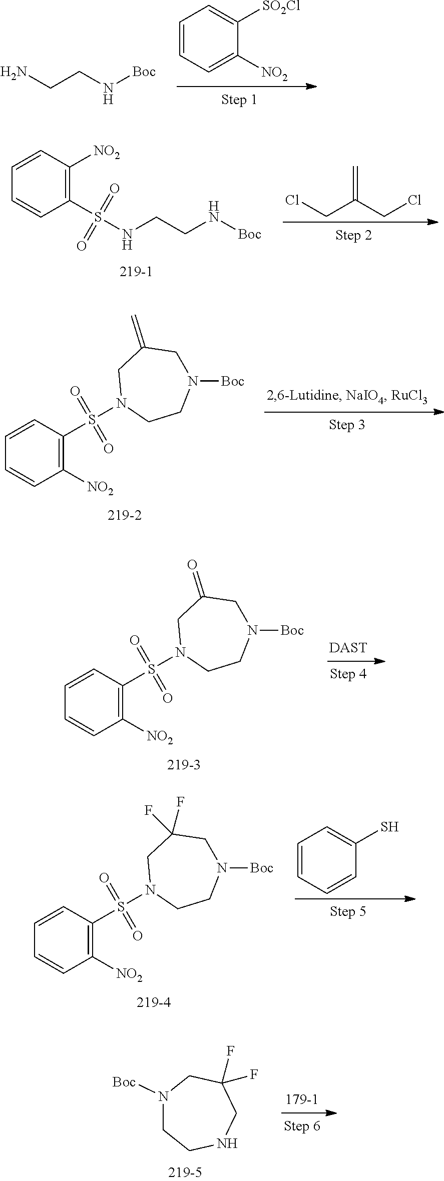

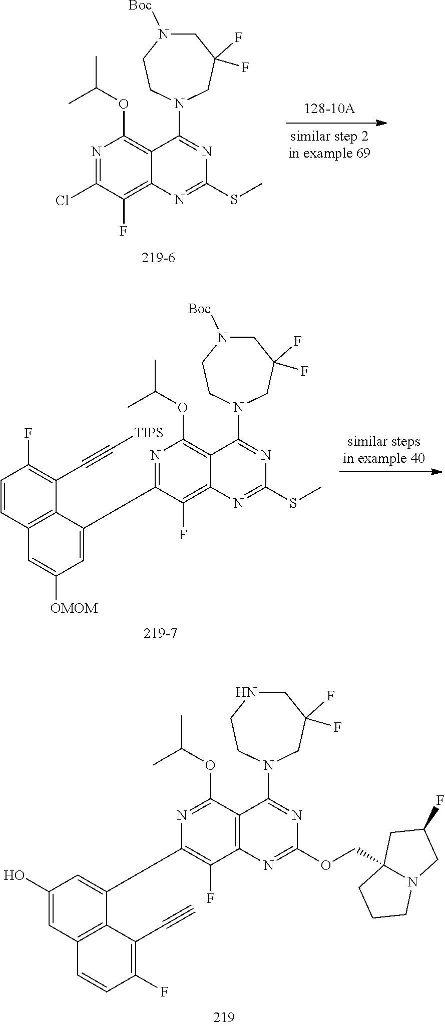

Step 1: To a solution of tert-butyl N-(2-aminoethyl)carbamate (4.35 g, 27.150 mmol) in dichloromethane (90 mL) was added triethyl amine (11.321 mL, 81.450 mmol). The mixture was stirred at 0° C. under N₂ for 10 min. 2-nitrobenzene-1-sulfonyl chloride (6.62 g, 29.865 mmol) was added. And the reaction was stirred at room temperature for 1 hour. The reaction mixture was diluted with water, extracted with dichloromethane. The organic layer was separated, washed with brine, and concentrated. The residue was purified through silica gel column (20 g, eluted by ethyl acetate in petroleum ether from 50% to 60%) to give 219-1.

Step 2: To a solution of 3-chloro-2-(chloromethyl)prop-1-ene (4.27 g, 34.125 mmol) in N,N-dimethylformamide (100 mL) was added sodium hydride (2.10 g, 52.500 mmol) at 0° C. The mixture was stirred at 0° C. under N₂ for 10 min. Then 219-1 (9.10 g, 26.348 mmol) in tetrahydrofuran (100 mL) was added to the 3-chloro-2-(chloromethyl)prop-1-ene solution at 0° C., the reaction was stirred at 60° C. for 1 hour. The reaction mixture was quenched with saturated ammonium chloride solution, diluted with water, extracted with ethyl acetate. The organic layer was separated, washed with brine, and concentrated. The residue was purified through silica gel column (eluted by ethyl acetate in petroleum ether from 25% to 30%) to give 219-2.

Step 3: To a solution of 219-2 (3.68 g, 9.259 mmol) in dichloromethane (50 mL) and acetonitrile (50 mL) was added 2,6-dimethylpyridine (2.0 g, 18.518 mmol), sodium periodate (7.9 g, 37.036 mmol, in 80 mL water), the mixture was stirred at room temperature for 10 min under N₂. Then Rhodium (III) chloride hydrate (5.3 mg, 0.020 mmol in 10 mL water) as added dropwise. The reaction was stirred at room temperature for 2 hours. The reaction mixture was quenched with saturated ammonium chloride solution, diluted with water, extracted with dichloromethane. The organic layer was separated, washed with brine, and concentrated. The residue was purified through silica gel column (eluted by ethyl acetate in petroleum ether from 20% to 25%) to give 219-3.

Step 4: To a solution of 219-3 (1.5 g, 3.755 mmol) in dichloromethane (50 mL) was added diethylaminosulfur trifluoride (3.0 g, 18.777 mmol) dropwise at 0° C. under N₂. And the reaction was stirred at room temperature for 18 hours. The reaction mixture was quenched with saturated aqueous sodium bicarbonate solution, diluted with water, extracted with dichloromethane. The organic layer was separated, washed with brine, and concentrated. The residue was purified through silica gel column (eluted by ethyl acetate in petroleum ether from 25% to 35%) to give 219-4.

Step 5: To a solution of 219-4 (1.47 g, 3.488 mmol) in acetonitrile (40 mL) was added sodium phenylsulfanide (1.4 g, 10.465 mmol), potassium carbonate (1.4 g, 10.465 mmol) at 0° C. under N₂. The reaction was stirred at 40° C. for 1 hour. The reaction was cooled to room temperature, diluted with water, extracted with dichloromethane. The organic layer was separated, washed with brine, and concentrated. The residue was purified through silica gel column (eluted by ethyl acetate in petroleum ether from 25% to 35%) to give 219-5.

Step 6: To a solution of 179-1 (450 mg, 1.481 mmol) in acetonitrile (20 mL) was added 219-5 (455.02 mg, 1.926 mmol), (benzotriazol-1-yloxy)tripyrrolidinophosphonium hexafluorophosphate (1156.4 mg, 2.222 mmol), and triethylamine (749.6 mg, 7.407 mmol), and the reaction was stirred at 60° C. for 1 hour. The reaction was cooled to room temperature, diluted with water, extracted with ethyl acetate. The organic layer was separated, washed with brine, concentrated. The residue was purified through silica gel column (eluted by ethyl acetate in petroleum ether from 10% to 15%) to give 219-6.

Compound 219-7 was prepared from 219-6 and 128-10A following the procedure for the synthesis of compound 217-1 in example 69.

Compound 219 was prepared from 219-7 following the procedure for the synthesis of compound 128 in example 40 as a 3 eq of TFA salt. LCMS (ESI, m/z): [M+H]⁺=683.3; ¹H NMR (400 MHz, methanol-d₄, ppm): δ 7.89 (dd, J=9.2, 5.6 Hz, 1H), 7.37-7.32 (m, 2H), 7.25 (d, J=2.4 Hz, 1H), 5.67-5.54 (m, 2H), 4.73-4.63 (m, 2H), 4.53-4.44 (m, 1H), 4.41-4.33 (m, 1H), 4.22-4.16 (m, 1H), 4.11-4.04 (m, 1H), 3.99-3.79 (m, 6H), 3.76-3.70 (m, 1H), 3.54-3.46 (m, 1H), 3.28 (d, J=2.4 Hz, 1H), 2.74-2.60 (m, 2H), 2.47-2.35 (m, 3H), 2.24-2.19 (m, 1H), 1.48-1.41 (m, 6H). ¹⁹F NMR (376 MHz, methanol-d₄, ppm): δ −98.30 (2F), −111.76 (1F), −151.38 (1F), −174.30 (1F).

### Example 72 Synthesis of Compound 220

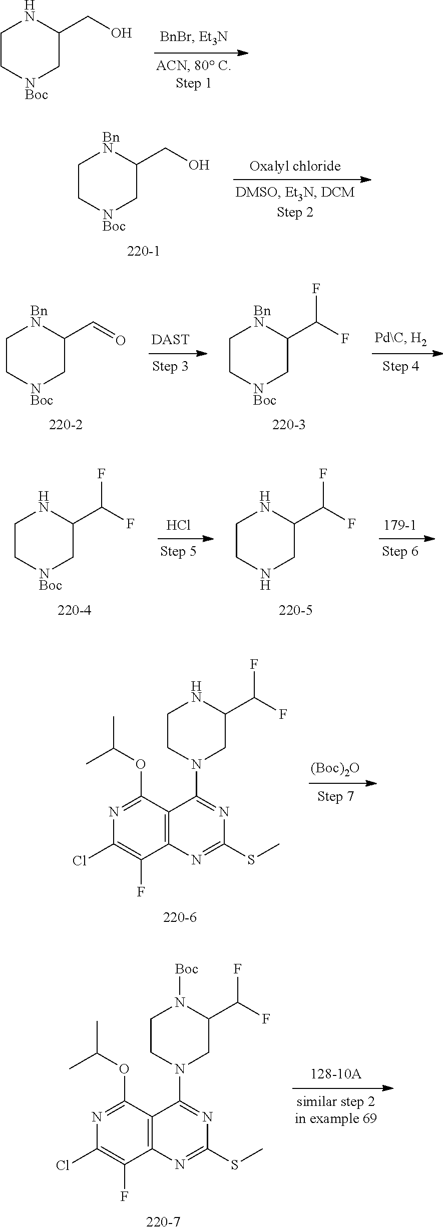

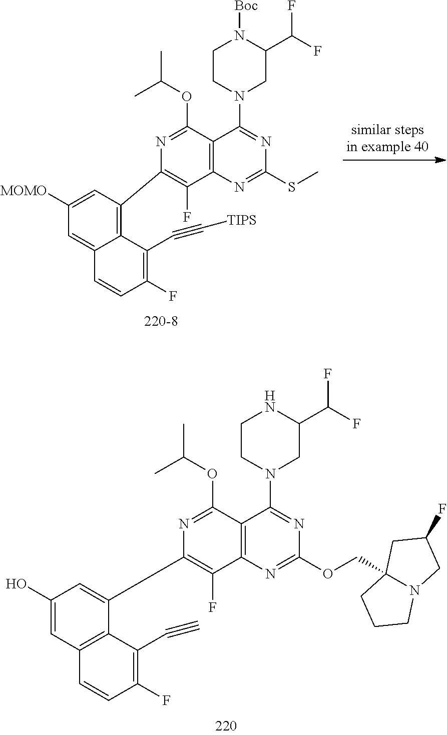

Step 1: A mixture of tert-butyl 3-(hydroxymethyl) piperazine-1-carboxylate (10 g, 46.236 mmol), (bromomethyl)benzene (9.49 g, 55.484 mmol) and triethylamine (12.853 mL, 92.473 mmol) in acetonitrile (100 mL) was stirred at 80° C. for 16 hours. The resulting solution was concentrated and purified by silica gel chromatography (ethyl acetate/petroleum ether=1/5) to afford 220-1.

Step 2: Dimethyl sulfoxide (3.077 mL, 43.324 mmol) was added to a −78° C. solution of oxalyl chloride (2.382 mL, 28.149 mmol) in dichloromethane (70 mL), then stirred 15 minutes. 220-1 (7.5 g, 24.477 mmol) in dichloromethane (10 mL) was slowly added, stirred at −78° C. for 1 hour. Triethylamine (16.331 mL, 117.490 mmol) was added, and the reaction mixture was stirred at room temperature for another 1 hour. The resulting mixture was diluted with dichloromethane, washed with water and brine. The organic layer was concentrated and dried under vacuum to afford 220-2.

Step 3: A solution of 220-2 (6.7 g, 22.011 mmol) in dichloromethane (50 mL) was added diethyl-aminosulfur trifluoride (7.1 g, 44.022 mmol) at 0° C., then stirred for 2 hours at 0° C. The resulting mixture was poured into ice water and extracted with dichloromethane. The organic layer was washed with water and brine, concentrated and purified by silica gel chromatography (ethyl acetate/petroleum ether=1/5) to afford 220-3.

Step 4: A mixture of 220-3 (2.5 g, 7.660 mmol) and 10% Pd\C (0.3 g) in methanol (50 mL) was stirred at room temperature for 16 hours under hydrogen atmosphere. The resulting mixture was filtered and washed with dichloromethane/methanol (10/1), concentrated and dried under vacuum to afford 220-4, which was used next step directly.

Step 5: A solution of 220-4 (1.8 g, crude) and 4 M HCl in ethyl acetate (20 mL) was stirred at room temperature for 1 hour. The resulting mixture was concentrated and dried under vacuum to afford 220-5, which was used next step directly.

Step 6: A mixture of 179-1 (1.78 g, 5.860 mmol), benzotriazol-1-yl-oxytripyrrolidinophosphonium hexafluorophosphate (4.6 g, 8.790 mmol), 1,8-diazabicyclo [5.4.0] undec-7-ene (1.8 g, 11.8 mmol) in N, N-dimethylformamide (20 mL) was stirred at 0° C. for 10 minutes. A solution of 220-5 (1.2 g, 8.790 mmol) and 1,8-diazabicyclo [5.4.0]undec-7-ene (1.8 g, 11.8 mmol) in N, N-dimethylformamide (2 mL) was added into above solution, stirred at 0° C. for another 1 hour. The reaction was quenched with water. A solid was precipitated and filtered to afford 220-6.

Step 7: 220-6 was dissolved with tetrahydrofuran (30 mL) and di-tert-butyl dicarbonate (2.6 g, 11.852 mmol), sodium bicarbonate (1.5 g, 17.778 mmol) was added, stirred room temperature for 16 hours. The resulting mixture was quenched with water, extracted with ethyl acetate. The combined the organic layers were washed with brine, evaporated to dryness and purified by flash column chromatography (ethyl acetate/petroleum ether=1/5) to give 220-7.

Compound 220-8 was prepared from 220-7 and 128-10A following the procedure for the synthesis of compound 217-1 in example 69.

Compound 220 was prepared from 220-8 following the procedure for the synthesis of compound 128 in example 40 as a 2 eq of TFA salt. LCMS (ESI, m/z): [M+H]⁺=683.6; ¹H NMR (400 MHz, methanol-d₄, ppm): δ 7.86 (dd, J=9.2, 5.7 Hz, 1H), 7.40-7.28 (m, 2H), 7.22-7.15 (m, 1H), 6.34 (t, J=53.7 Hz, 1H), 5.67-5.47 (m, 2H), 4.76-4.62 (m, 3H), 4.50-4.36 (m, 1H), 4.07-3.82 (m, 4H), 3.72-3.52 (m, 3H), 3.51-3.33 (m, 3H), 2.78-2.50 (m, 2H), 2.47-2.27 (m, 3H), 2.23-2.10 (m, 1H), 1.47-1.35 (m, 6H). ¹⁹F NMR (376 MHz, methanol-d₄, ppm): −111.81 (1F), −129.26 (2F), −151.08 (1F), −174.26 (1F).

### Example 73 Synthesis of Compound 224

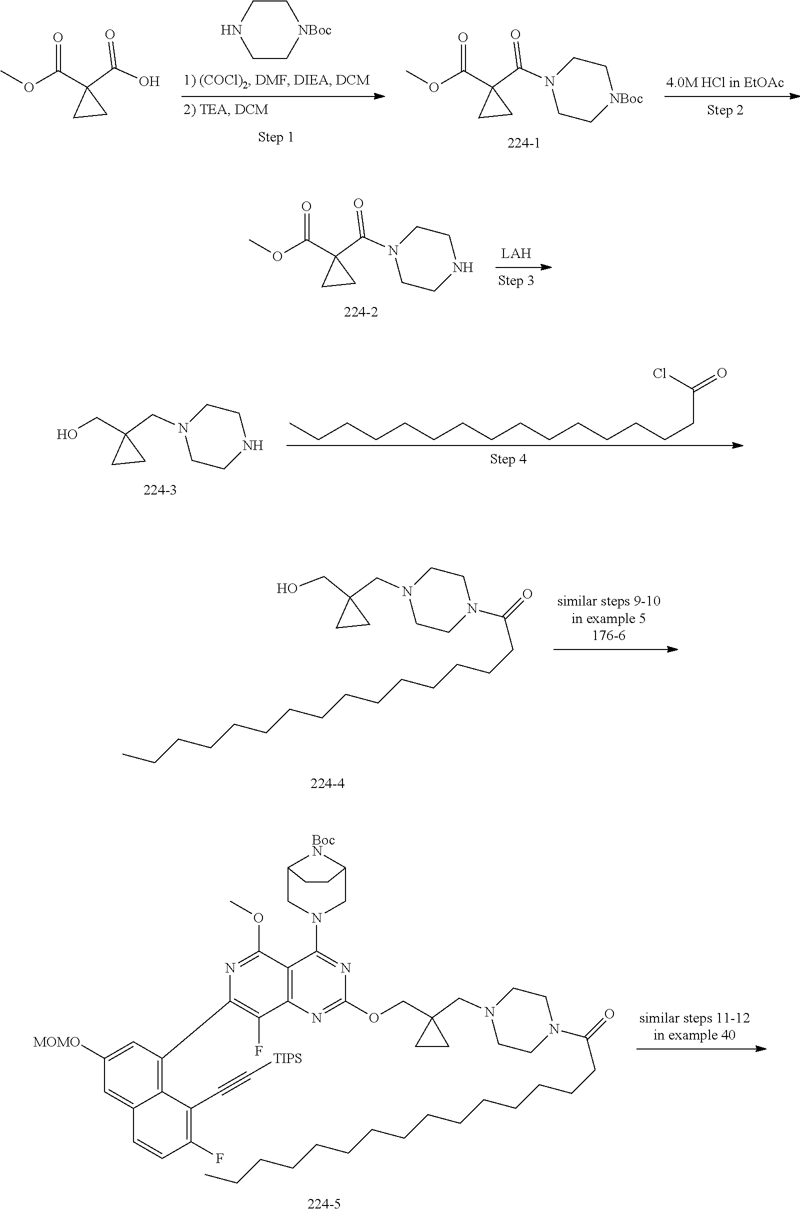

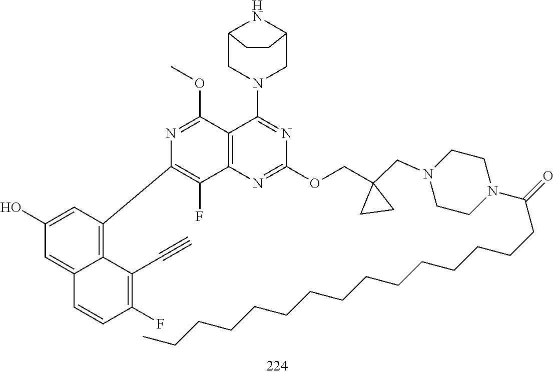 [SMILES: C#Cc1c(F)ccc2cc(O)cc(-c3nc(OC)c4c(N5CC6CCC(C5)N6)nc(OCC5(CN6CCN(C(=O)CCCCCCCCCCCCCCC)CC6)CC5)nc4c3F)c12]

Step 1: To a solution of 1-(methoxycarbonyl) cyclopropane-1-carboxylic acid (15.9 g, 110.3 mmol) in dichloromethane (80 mL) was added oxalic dichloride (12.3 mL, 145.6 mmol) at room temperature, the mixture was stirred for 2 hours. The reaction mixture was concentrated to give a residue, the residue was added dropwise to a solution of tert-butyl piperazine-1-carboxylate (26.3 g, 141.2 mmol) and triethylamine (30.7 mL, 220.6 mmol) in dichloromethane (80 mL). The reaction mixture was stirred for 0.5 hours and quenched with water. The organic layer was collected, dried over sodium sulfate, filtered and concentrated. The residue was purified by column chromatography on silica gel (dichloromethane to dichloromethane/methanol=0-10%) to give 224-1.

Step 2: To a solution of 224-1 (15.7 g, 50.3 mmol) in ethyl acetate (50 mL) wad added 4N hydrochloric acid in ethyl acetate (80 mL) at 0° C., and the reaction was stirred at room temperature for 16 hours. The reaction mixture was concentrated, the residue was suspended in ethyl acetate. The mixture was adjusted to pH to 12 by triethylamine and filtered. The filtrate was concentrated under reduced pressure to give 224-2.

Step 3: To a solution of 224-2 (4.3 g, 20.26 mmol) in tetrahydrofuran (40 mL) wad added lithium aluminum hydride (1.92 g, 50.6 mmol) at 0° C., and the reaction was stirred at room temperature for 2 hours, then stirred at 60° C. for 0.5 hour. The reaction mixture was cooled to −20° C., treated with ethyl acetate (160 mL), followed by the addition of sodium sulfate decahydrate. Then mixture was stirred at room temperature for 0.5 hour. The suspension was filtered and rinsed with ethyl acetate. The organic layers were combined, dried over sodium sulfate, filtered and concentrated to afford 224-3.

Step 4: To a solution of 224-3 (340 mg, 2.00 mmol) and triethylamine (0.69 mL, 4.99 mmol) in THF (10 mL) was added hexadecanoyl chloride (494.0 mg, 1.80 mmol) at 0° C., then stirred at 0° C. for 1 hour under N₂ atmosphere. The mixture was diluted with ethyl acetate and saturated aqueous sodium bicarbonate, extracted with ethyl acetate. The combined organic layers were washed with brine, dried over anhydrous sodium sulfate, filtered and concentrated. The residue was purified by column chromatography on silica gel (petroleum ether to petroleum ether/ethyl acetate=1/1) to give 224-4.

Compound 224-5 was prepared from 176-6 and 224-4 following the procedure for the synthesis of compound 60-10 in example 5.

Compound 224 was prepared from 224-5 following the procedure for the synthesis of compound 128 in example 40 as a 3 eq of TFA salt. LCMS (ESI, m/z): [M+H]⁺=881.4; ¹H NMR (400 MHz, methanol-d₄, ppm): δ 7.89-7.82 (m, 1H), 7.38-7.26 (m, 2H), 7.26-7.15 (m, 1H), 4.54-4.38 (m, 4H), 4.27-4.18 (m, 2H), 4.06 (s, 3H), 4.00-3.46 (m, 6H), 3.42-3.32 (m, 4H), 3.29-3.24 (m, 1H), 3.22-2.62 (m, 2H), 2.44-2.35 (m, 2H), 2.22-2.06 (m, 4H), 1.61-1.50 (m, 2H), 1.32-1.23 (m, 24H), 1.02-0.94 (m, 2H), 0.93-0.80 (m, 5H). ¹⁹F NMR (376 MHz, methanol-d₄, ppm): δ −111.8 (1F), −149.9 (1F).

### Example 74 Synthesis of Compound 212

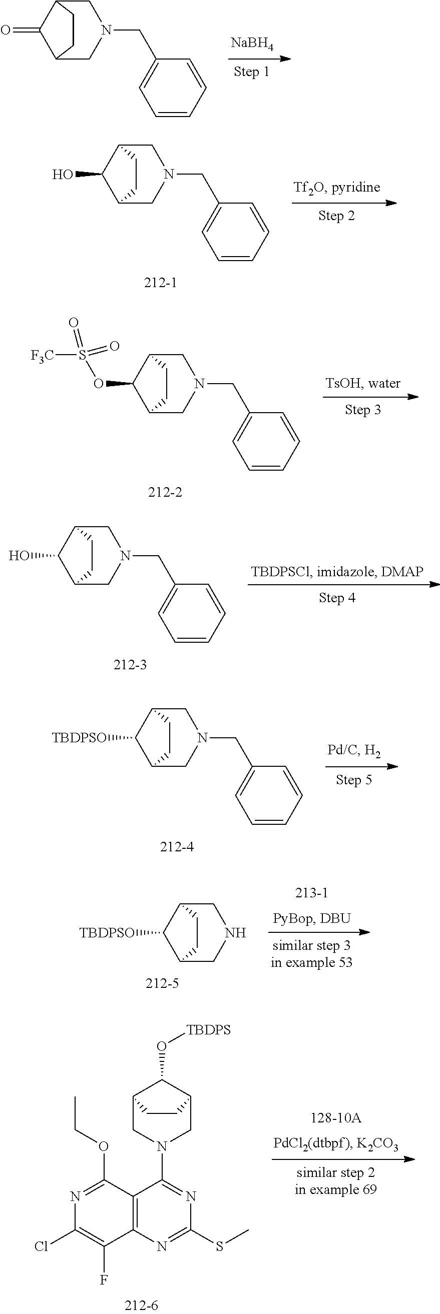

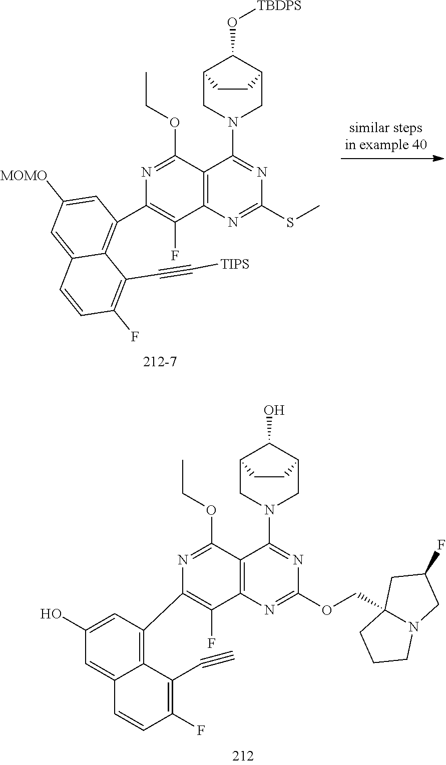

Step 1: To a stirred light brown solution of 3-benzyl-3-azabicyclo[3.2.1]octan-8-one (4.0 g, 18.58 mmol) in methanol (50 mL) was added NaBH₄ (914 mg, 24.15 mmol) in portions slowly at 0-5° C. The reaction was stirred at 0-5° C. for 1 hour. The reaction was quenched by water, extracted by DCM, dried over Na₂SO₄, filtered, concentrated, dried under vacuum to give 212-1.

Step 2: To a stirred solution of 212-1 (3.7 g, 17.03 mmol) in DCM (25 mL)/pyridine (6.73 g, 85.1 mmol) was added Tf₂O (9.62 g, 34.1 mmol) dropwise at 0-5° C. The reaction was stirred at this temperature for 30 mins. The reaction mixture was washed with sat. NaHCO₃, dried over Na₂SO₄, filtered, concentrated, dried under vacuum to give 212-2.

Step 3: To the solution of 212-2 (1.36 g, 3.89 mmol) in toluene (20 mL) was added DMSO (8 mL), water (2 mL) and TsOH·H₂O (1.1 g, 5.79 mmol). The reaction was stirred at 100° C. for 3 days. The reaction mixture was diluted with sat. K₂CO₃, extracted by ethyl acetate, concentrated. The residue was purified through silica gel column (eluted by ethyl acetate in petroleum ether (2% TEA) from 0% to 35% in 20 mins) to give 212-3.

Step 4: To a solution of 212-3 (1.8 g, 8.283 mmol) and 1H-imidazole (1.396 mL, 20.708 mmol) and N,N-dimethylpyridin-4-amine (0.2 g, 1.657 mmol) in N,N-dimethylformamide (15 mL) were added tert-butyl(chloro)diphenylsilane (3.222 mL, 12.425 mmol) at room temperature. And the reaction was stirred at 60° C. for 18 hours. The reaction was diluted with ethyl acetate and washed with water. The organic layer was separated, dried over Na₂SO₄, filtered and concentrated in vacuo. The residue was purified through silica gel column chromatography (eluted with ethyl acetate in petroleum ether from 0 to 50% in 20 mins to afford the title compound 212-4.

Step 5: To a solution of 212-4 (2.50 g, 5.486 mmol) in ethyl acetate (20 mL) were added acetic acid (5 mL) and palladium (0.2 g, 0.188 mmol). The reaction was stirred at room temperature for 2.0 days under H₂. The reaction mixture was adjusted to pH to 7-8 by saturated aqueous sodium bicarbonate solution. It was diluted with water, extracted with ethyl acetate. The organic layer was separated, washed with brine, concentrated. The residue was purified through silica gel column (eluted by ethyl acetate in petroleum ether from 50% to 60%) to give 212-5.

Compound 212-6 was prepared from 213-1 and 212-5 following the procedure for the synthesis of compound 155-3 in example 53.

Compound 212-7 was prepared from 212-6 and 128-10A following the procedure for the synthesis of compound 217-1 in example 69.

Compound 212 was prepared from 212-7 following the procedure for the synthesis of compound 128 in example 40 as a 2 eq of TFA salt. LCMS (ESI, m/z): [M+H]⁺=660.2; ¹H NMR (400 MHz, methanol-d₄, ppm): δ 7.88 (dd, J=9.2, 5.6 Hz, 1H), 7.38-7.32 (m, 2H), 7.26 (d, J=2.4 Hz, 1H), 5.69-5.48 (m, 1H), 4.76-4.67 (m, 2H), 4.62-4.49 (m, 3H), 4.40-4.32 (m, 1H), 4.20 (s, 1H), 4.12-3.82 (m, 4H), 3.68-3.41 (m, 3H), 2.69-2.61 (m, 1H), 2.43-2.20 (m, 6H), 2.19-2.10 (m, 1H), 1.97-1.85 (m, 2H), 1.60-1.48 (m, 1H), 1.43 (t, J=7.20 Hz, 3H), 1.40-1.30 (m, 1H). ¹⁹F NMR (376 MHz, methanol-d₄, ppm): δ −111.70 (1F), −152.10 (1F), −174.06 (1F).

### Example 75 Synthesis of Compound 230

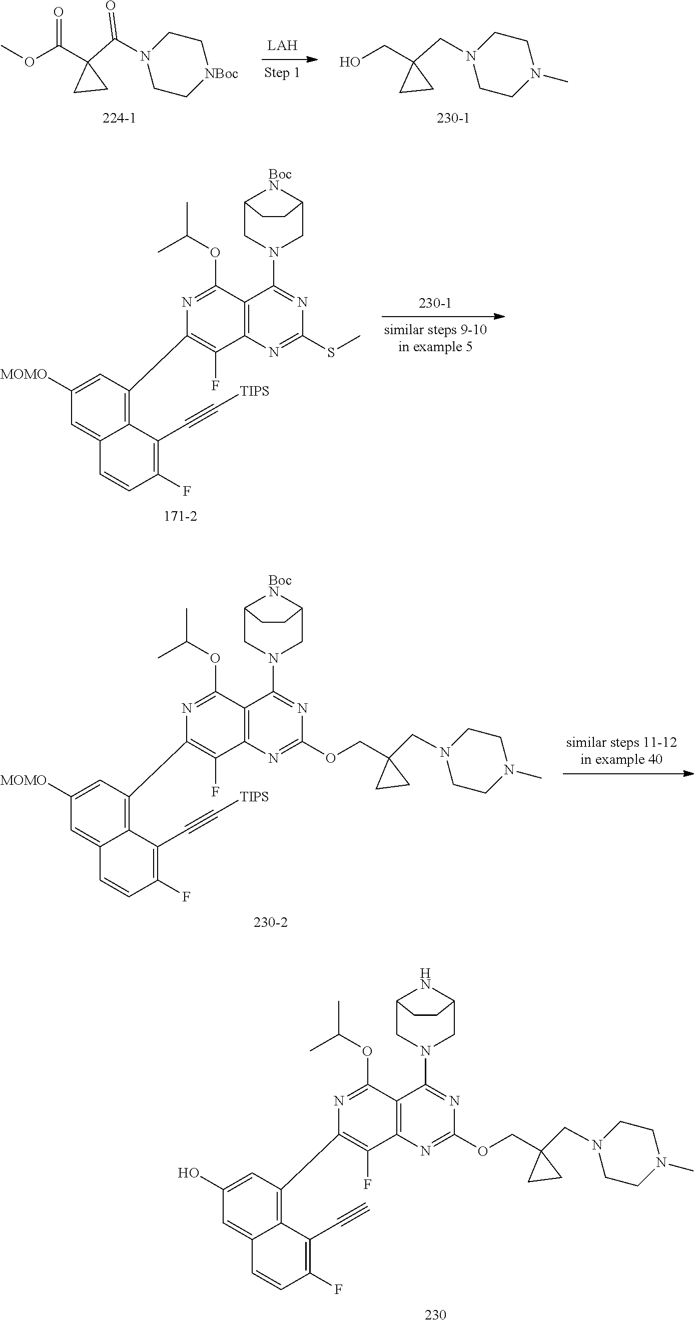

Step 1: To a solution of 224-1 (1.74 g, 5.57 mmol) in tetrahydrofuran (10 mL) was added lithium aluminum hydride (634.2 mg, 16.7 mmol) at 0° C., and the reaction was stirred at room temperature for 2 hours, then stirred at 60° C. for 0.5 hour. The reaction mixture was cooled to −20° C., treated with ethyl acetate, followed by the addition of water and 15% sodium hydroxide aqueous. Then mixture was stirred at room temperature for 0.5 hour, then filtered and rinsed with dichloromethane. The filtrate was extracted with dichloromethane. The organic layers were combined, dried over sodium sulfate, filtered and concentrated to afford the title compound 230-1.

Compound 230-2 was prepared from 171-2 and 230-1 following the procedure for the synthesis of compound 60-10 in example 5.

Compound 230 was prepared from 230-2 following the procedure for the synthesis of compound 128 in example 40 as a 3 eq of TFA salt. LCMS (ESI, m/z): [M+H]⁺=684.5; ¹H NMR (400 MHz, methanol-d₄, ppm): δ 7.87-7.83 (m, 1H), 7.36-7.29 (m, 2H), 7.24-7.13 (m, 1H), 5.64-5.47 (m, 1H), 4.60-4.35 (m, 4H), 4.25-4.14 (m, 2H), 3.88-3.77 (m, 2H), 3.41 (s, 1H), 3.30-3.18 (m, 8H), 2.78 (s, 3H), 2.76-2.70 (m, 1H), 2.65-2.58 (m, 1H), 2.13-1.97 (m, 4H), 1.46-1.38 (m, 6H), 0.85-0.75 (m, 2H), 0.65-0.55 (m, 2H). ¹⁹F NMR (376 MHz, methanol-d₄, ppm): δ −111.98 (1F), −150.95 (1F).

### Example 76 Synthesis of Compound 231

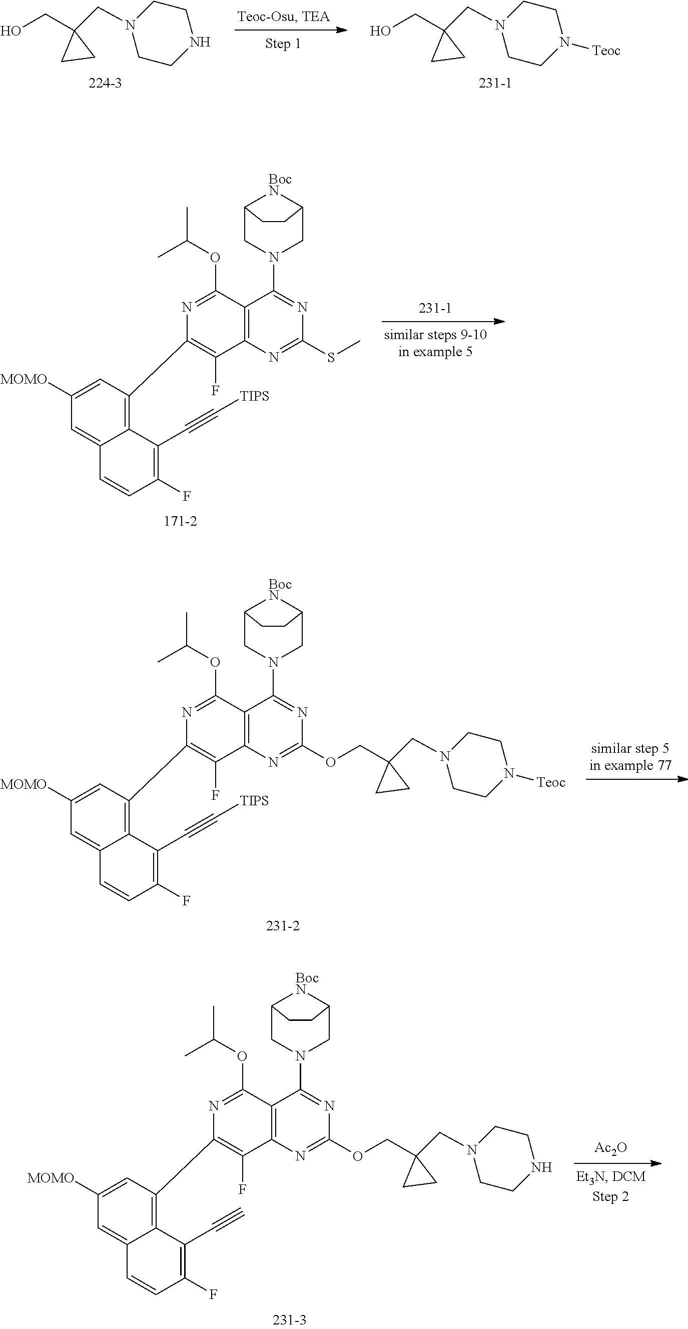

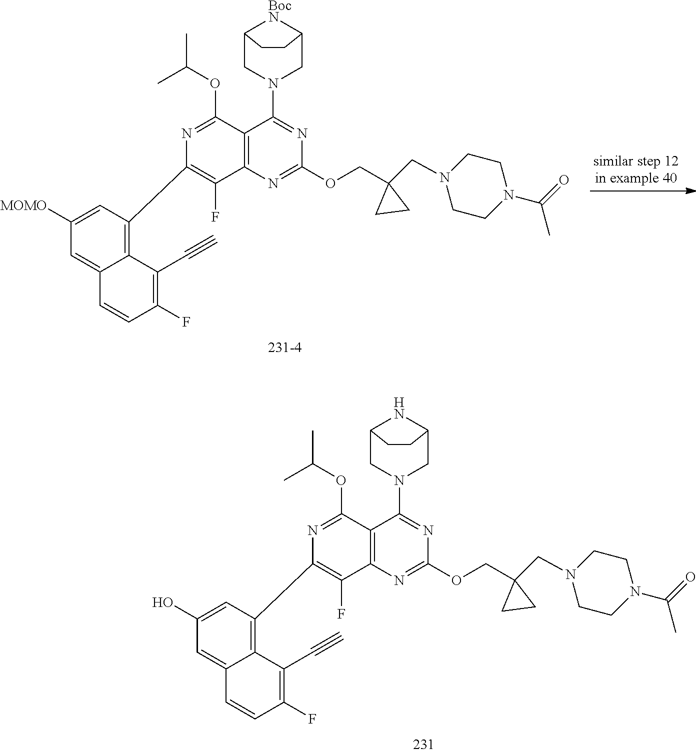

Step 1: To a solution of 224-3 (1.02 g) in tetrahydrofuran (15 mL) was added triethylamine (1.66 mL, 11.98 mmol) and 1-({[2-(trimethylsilyl)ethoxy]carbonyl}oxy)pyrrolidine-2,5-dione (1.86 g, 7.19 mmol) at 0° C. The reaction was stirred at room temperature for 2 hours. The mixture was diluted with ethyl acetate, washed with water. The organic layer was concentrated. The residue was was purified by silica gel chromatography (petroleum ether to petroleum ether/ethyl acetate=0˜50%) to give 231-1.

Compound 231-2 was prepared from 171-2 and 231-1 following the procedure for the synthesis of compound 60-10 in example 5.

Compound 231-3 was prepared from 231-2 following the procedure for the synthesis of compound 246-7 in example 77.

Step 2: A mixture of 231-3 (100 mg, 0.123 mmol) and triethylamine (0.026 mL, 0.184 mmol) in dichloromethane (10 mL) was added acetic anhydride (0.013 mL, 0.135 mmol) at 0° C., stirred at 0° C. for 1 hour. The resulting mixture was washed with water, and concentrated. The residue was purified by silica gel chromatography (petroleum ether/ethyl acetate=5/1) to afford 231-4.

Compound 231 was prepared from 231-4 following the procedure for the synthesis of compound 128 in example 40 as a 4 eq of TFA salt. LCMS (ESI, m/z): [M+H]⁺=712.2; ¹H NMR (400 MHz, methanol-d₄, ppm): δ 7.85 (dd, J=9.2, 5.6 Hz, 1H), 7.37-7.27 (m, 2H), 7.22-7.12 (m, 1H), 5.63-5.44 (m, 1H), 4.78-4.64 (m, 2H), 4.57-4.41 (m, 4H), 4.27-4.14 (m, 2H), 4.05-3.67 (m, 5H), 3.60-3.32 (m, 6H), 2.21-1.92 (m, 7H), 1.41 (dd, J=6.0, 3.6 Hz, 6H), 1.04-0.97 (m, 2H), 0.91-0.85 (m, 2H). ¹⁹F NMR (376 MHz, methanol-d₄, ppm): −111.97 (1F), −151.92 (1F).

### Example 77 Synthesis of Compound 246

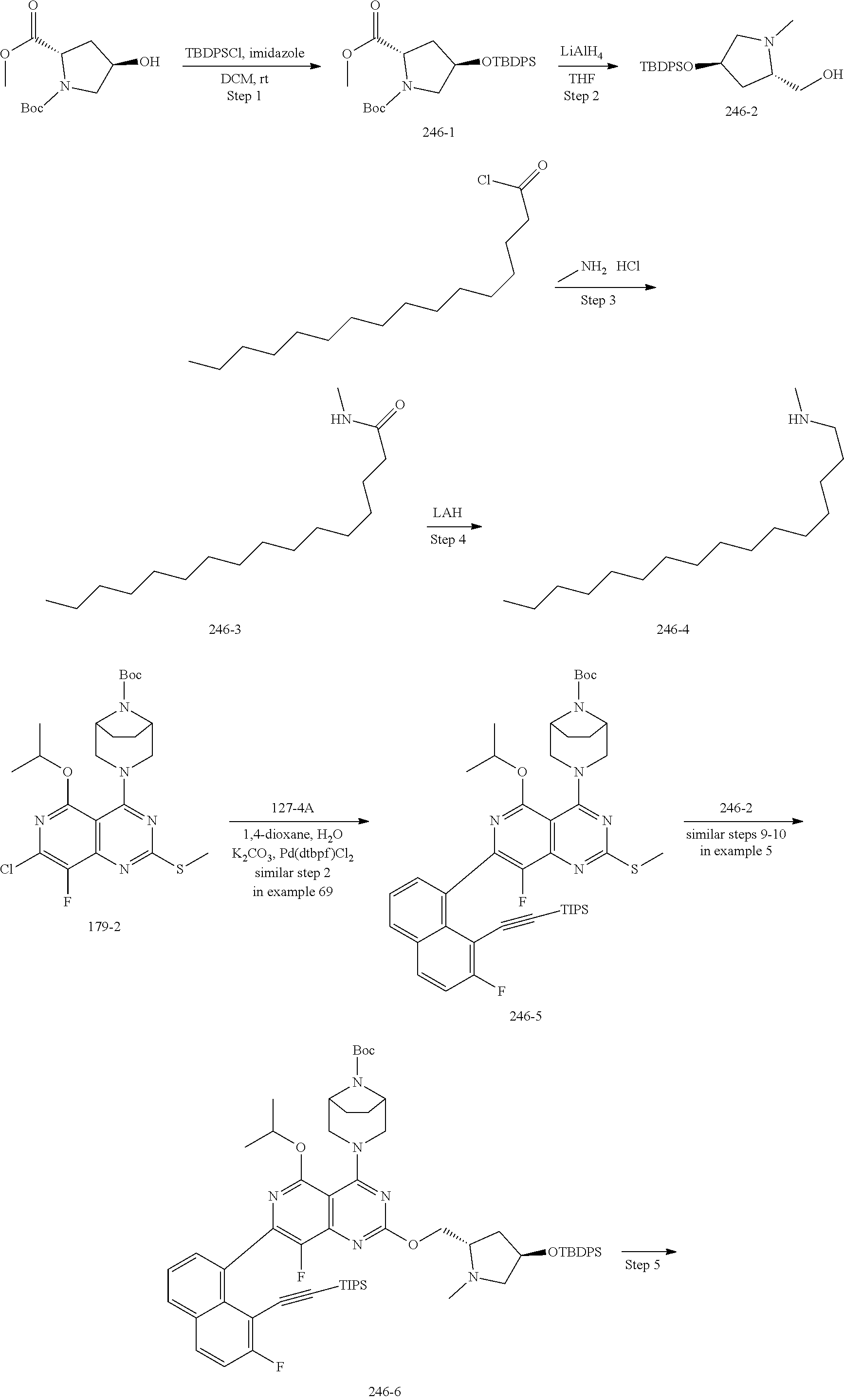

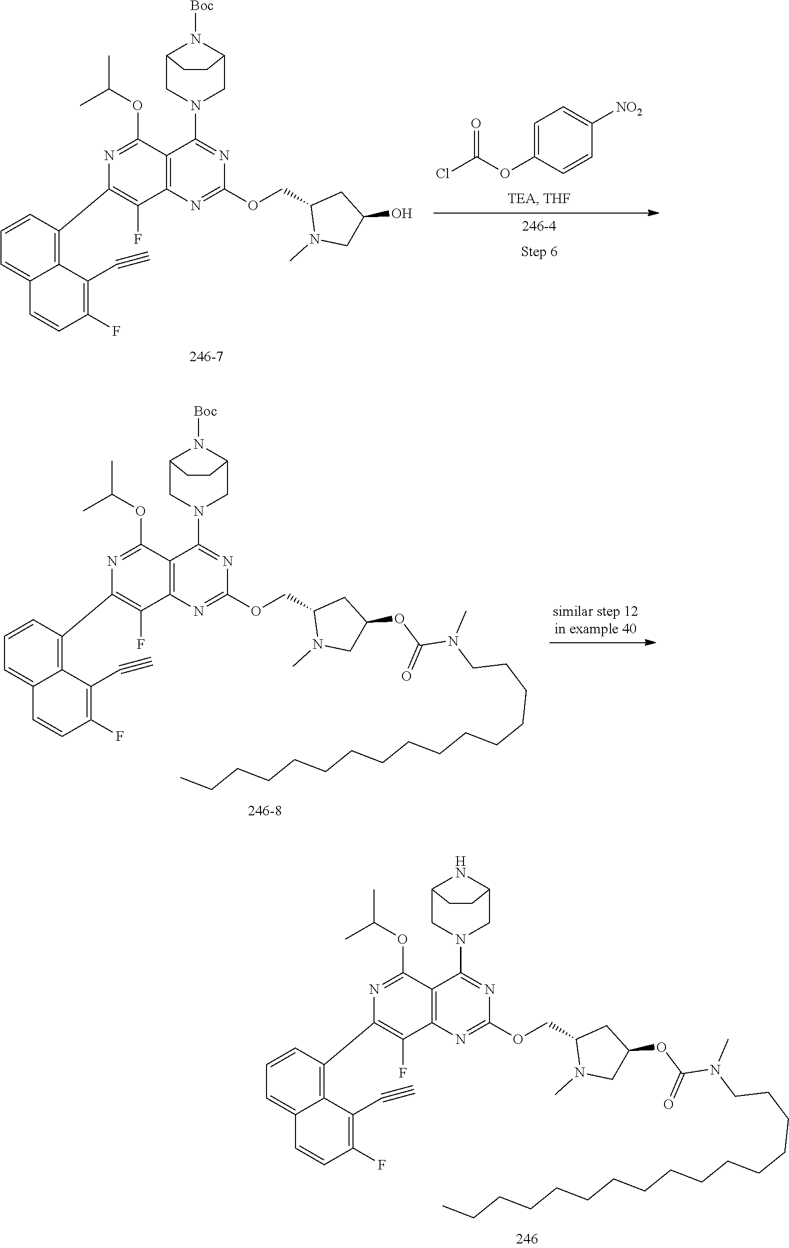 [SMILES: C#Cc1c(F)ccc2cccc(-c3nc(OC(C)C)c4c(N5CC6CCC(C5)N6C(=O)OC(C)(C)C)nc(OC[C@@H]5C[C@@H](O)CN5C)nc4c3F)c12 ; O=C(Cl)Oc1ccc([N+](=O)[O-])cc1 ; C#Cc1c(F)ccc2cccc(-c3nc(OC(C)C)c4c(N5CC6CCC(C5)N6C(=O)OC(C)(C)C)nc(OC[C@@H]5C[C@@H](OC(=O)N(C)CCCCCCCCCCCCCCCC)CN5C)nc4c3F)c12 ; C#Cc1c(F)ccc2cccc(-c3nc(OC(C)C)c4c(N5CC6CCC(C5)N6)nc(OC[C@@H]5C[C@@H](OC(=O)N(C)CCCCCCCCCCCCCCCC)CN5C)nc4c3F)c12]

Step 1: To a solution of 1-(tert-butyl) 2-methyl (2S,4R)-4-hydroxypyrrolidine-1,2-dicarboxylate (10 g, 40.770 mmol) in dichloromethane (20 mL) was added imidazole (5.5 g, 81.539 mmol) and tert-butylchlorodiphenylsilane (16.8 g, 61.155 mmol) at 0° C., the reaction mixture was stirred at room temperature for 1.5 hours. The reaction was concentrated. The residue was diluted with ethyl acetate, washed with water and brine. The organic layers were dried over sodium sulfate, filtered, and concentrated. The residue was purified by flash column chromatography (10%-15% ethyl acetate in petroleum ether) to give the 246-1.

Step 2: To a solution of 246-1 (4.79 mg, 9.903 mmol) in tetrahydrofuran (40 mL) was added lithium aluminum hydride (1.1 g, 29.710 mmol) at 0° C. The reaction mixture was stirred at 70° C. for 3 hours. Then the mixture was cooled to room temperature, and sodium sulfate decahydrate (20 g) was added, stirred for 10 minutes, followed by sodium sulfate (20 g), filtered, and concentrated. The residue was purified by prep-HPLC (50% ACN:50% H₂O) to give the 246-2.

Step 3: To a solution of methanamine hydrochloride (10.1 g, 150.28 mmol) in dichloromethane (80 mL) was added triethylamine (20.5 mL, 147.337 mmol) and hexadecanoyl chloride (8.1 g, 29.47 mmol) at 0° C., the reaction mixture was stirred at room temperature for overnight. The mixture was diluted with dichloromethane, washed with 1N hydrogenchloride and brine. Organic layer was dried over sodium sulfate, filtered and concentrated to give the 246-3.

Step 4: To a solution of lithium aluminum hydride (400 mg, 10.02 mmol) in tetrahydrofuran (20 mL) was added 246-3 (2.0 g, 7.42 mmol) at 0° C., then the mixture was stirred at 80° C. for 3 hours. Then the mixture was cooled to room temperature, and sodium sulfate decahydrate (20 g) was added, stirred for 10 minutes, followed by sodium sulfate (20 g), filtered, and concentrated to give the 246-4.

Compound 246-5 was prepared from 179-2 and 127-4A following the procedure for the synthesis of compound 217-1 in example 69.

Compound 246-6 was prepared from 246-5 and 246-2 following the procedure for the synthesis of compound 60-10 in example 5.

Step 5: To a solution of 246-6 (490 mg, 0.442 mmol) in N, N-dimethylformamide (10 mL) was added caesium fluoride (1.34 g, 8.840 mmol) at room temperature, then stirred at 50° C. for 3 hours under N₂ atmosphere. The mixture was diluted with ethyl acetate, washed with water and brine, dried over sodium sulfate, filtered, concentrated. The residue was purified by column chromatography on silica gel (dichloromethane/methanol=10/1) to give 246-7.

Step 6: To a solution of 246-7 (107 mg, 0.15 mmol) in tetrahydrofuran (2 mL) was added triethylamine (0.16 mL, 1.125 mmol) and 4-nitrophenyl chloroformate (105.8 mg, 0.53 mmol) at room temperature. The mixture was stirred at room temperature overnight under N₂ atmosphere. Then 246-4 (191.6 mg, 0.75 mmol) was added at room temperature. The reaction mixture was stirred for 30 minutes. The mixture was diluted with ethyl acetate and washed with water, separated. The organic layer was dried over sodium sulfate, filtered and concentrated. The residue was purified by prep-HPLC (acetonitrile/0.05% TFA in water=5%-95%) to give 246-8.

Compound 246 was prepared from 246-8 following the procedure for the synthesis of compound 128 in example 40 as a 3 eq of TFA salt. LCMS (ESI, m/z): [M+H]⁺/2=448.9; ¹H NMR (400 MHz, methanol-d₄, ppm): δ 8.12-8.08 (m, 2H), 7.66-7.61 (m, 2H), 7.44 (t, J=9.2 Hz, 1H), 5.57-5.51 (m, 1H), 5.35-5.30 (m, 1H), 4.98-4.91 (m, 1H), 4.76-4.69 (m, 1H), 4.55-4.52 (m, 2H), 4.22-4.06 (m, 4H), 3.88-3.85 (m, 2H), 3.53-3.40 (m, 2H), 3.28-3.25 (m, 2H), 3.15 (s, 3H), 2.94-2.90 (m, 3H), 2.54-2.43 (m, 2H), 2.06-2.0 (m, 4H), 1.60-1.50 (m, 2H), 1.42-1.40 (m, 6H), 1.32-1.24 (m, 26H), 0.89-0.86 (m, 3H). ¹⁹F NMR (376 MHz, methanol-d₄, ppm): δ −107.03 (1F), −151.42 (1F).

### Example 78 Synthesis of Compound 232

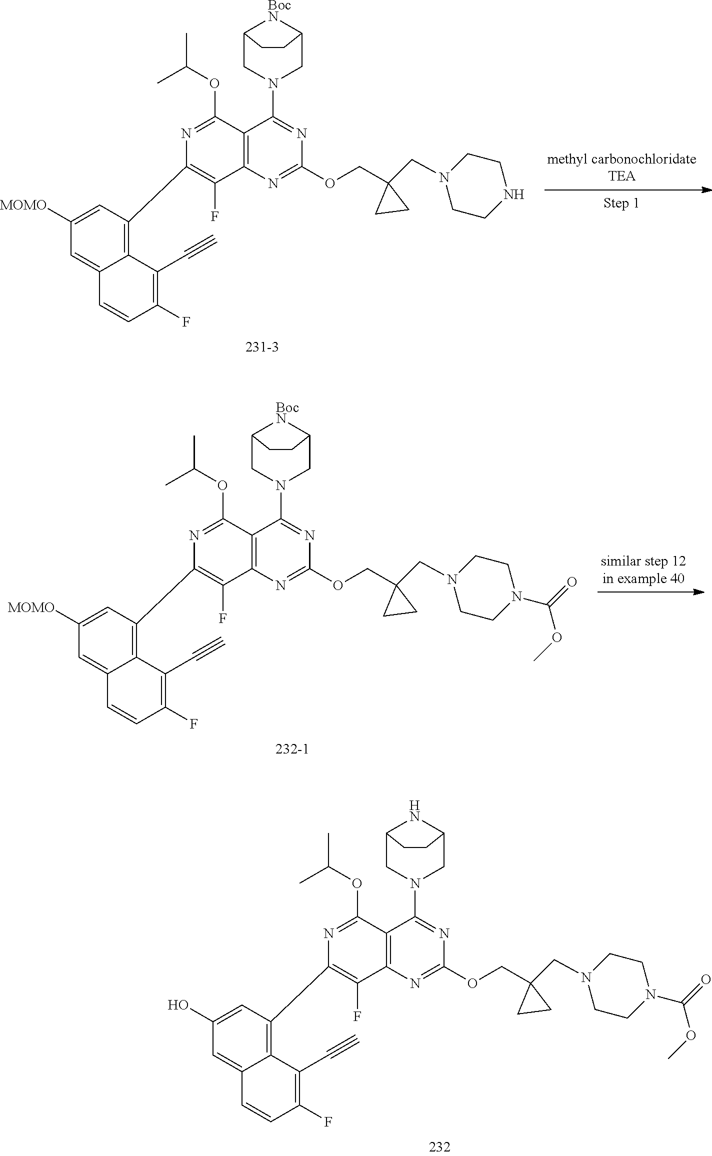

Step 1: To a solution of 231-3 (150 mg, 0.184 mmol) in dichloromethane (10 mL) was added triethylamine (21 mg, 0.369 mmol) and methyl chloroformate (21 mg, 0.221 mmol) at room temperature, then stirred for 0.5 hours under N₂ atmosphere. The mixture was diluted with ethyl acetate, washed with water and brine, dried over sodium sulfate, filtered, concentrated to give 232-1.

Compound 232 was prepared from 232-1 following the procedure for the synthesis of compound 128 in example 40 as a 3 eq of TFA salt. LCMS (ESI, m/z): [M+H]⁺=728.4; ¹H NMR (400 MHz, methanol-d₄, ppm): δ 7.88-7.81 (m, 1H), 7.34-7.27 (m, 2H), 7.21-7.17 (m, 1H), 5.59-5.48 (m, 1H), 4.66-4.28 (m, 4H), 4.25-4.15 (m, 2H), 3.88-3.81 (m, 2H), 3.75-3.68 (s, 3H), 3.45-3.38 (m, 2H), 3.34-3.32 (m, 1H), 3.31-3.28 (m, 8H), 2.16-1.94 (m, 4H), 1.46-1.35 (m, 6H), 1.05-0.92 (m, 2H), 0.92-0.84 (m, 2H). ¹⁹F NMR (376 MHz, methanol-d₄, ppm): δ −111.93 (1F), −150.10 (1F).

### Example 79 Synthesis of Compound 234

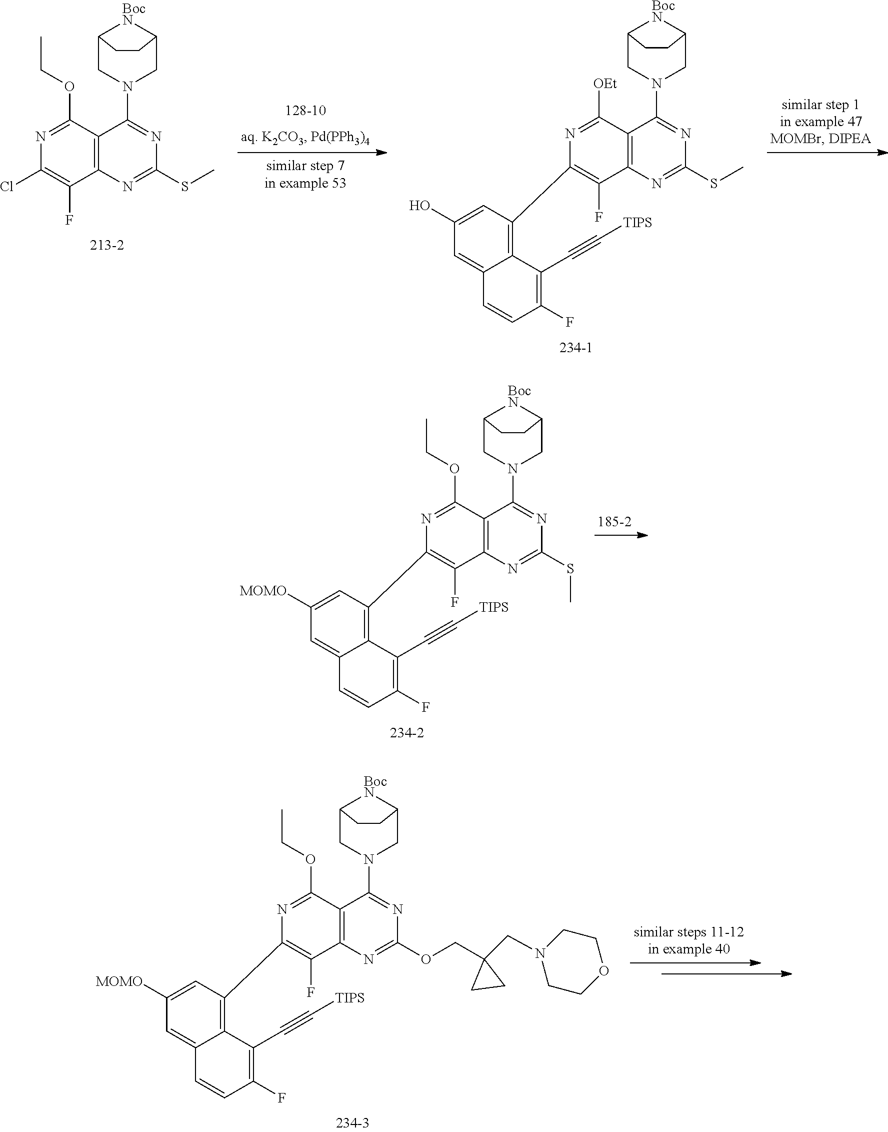

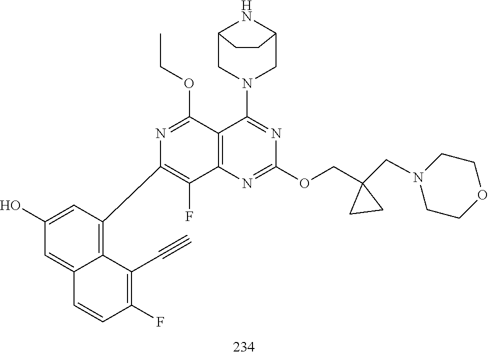 [SMILES: C#Cc1c(F)ccc2cc(O)cc(-c3nc(OCC)c4c(N5CC6CCC(C5)N6)nc(OCC5(CN6CCOCC6)CC5)nc4c3F)c12]

Compound 234-1 was prepared from 213-2 and 128-10 following the procedure for the synthesis of compound 155-7 in example 53.

Compound 234-2 was prepared from 234-1 following the procedure for the synthesis of compound 142-1 in example 47.

Compound 234-3 was prepared from 234-2 and 185-2 following the procedure for the synthesis of compound 60-10 in example 5.

Compound 234 was prepared from 234-3 following the procedure for the synthesis of compound 128 in example 40 as a 4 eq of TFA salt. LCMS (ESI, m/z): [M+H]⁺=657.4; ¹H NMR (400 MHz, methanol-d₄, ppm): δ 7.89-7.81 (m, 1H), 7.35-7.27 (m, 2H), 7.24-7.17 (m, 1H), 4.65-4.35 (m, 6H), 4.26-4.15 (m, 2H), 4.11-3.64 (m, 8H), 3.44-3.33 (m, 3H), 3.28-3.08 (m, 2H), 2.15-2.03 (m, 4H), 1.43 (t, J=7.2 Hz 3H), 1.05-0.93 (m, 2H), 0.92-0.83 (m, 2H). ¹⁹F NMR (376 MHz, methanol-d₄, ppm): δ −111.87 (1F), −150.43 (1F).

### Example 80 Synthesis of Compound 240

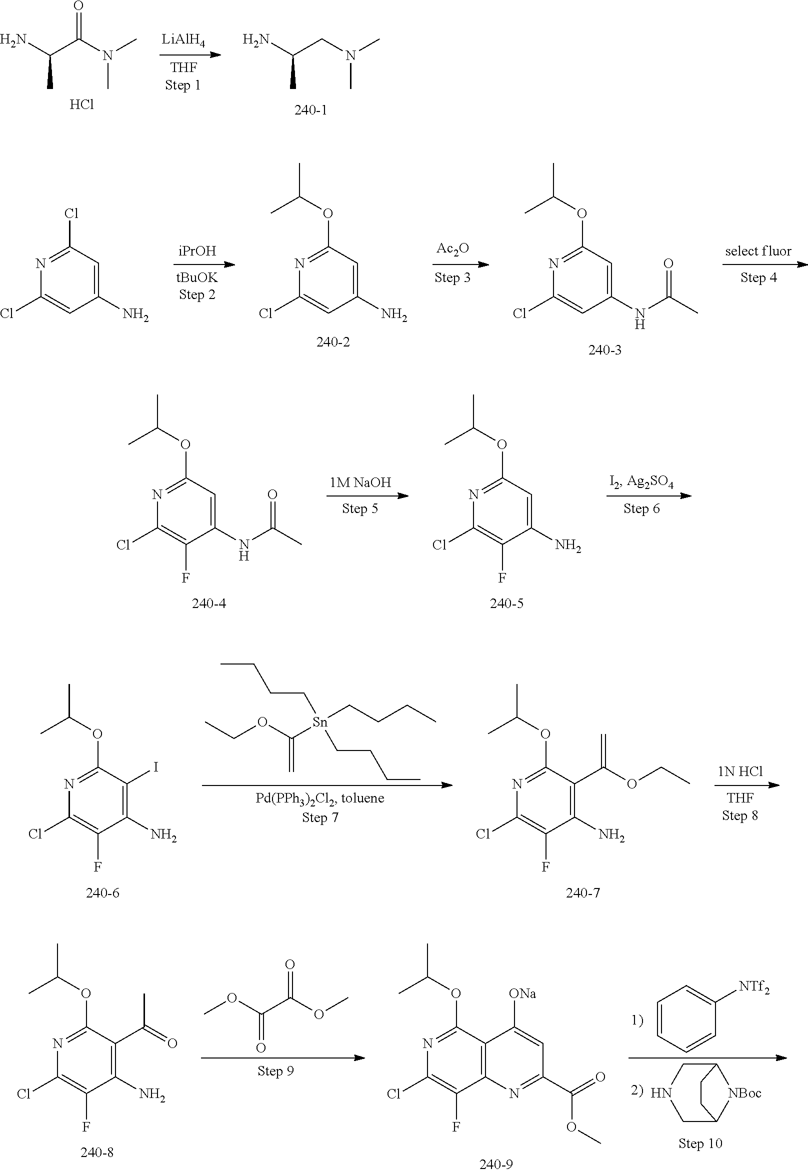

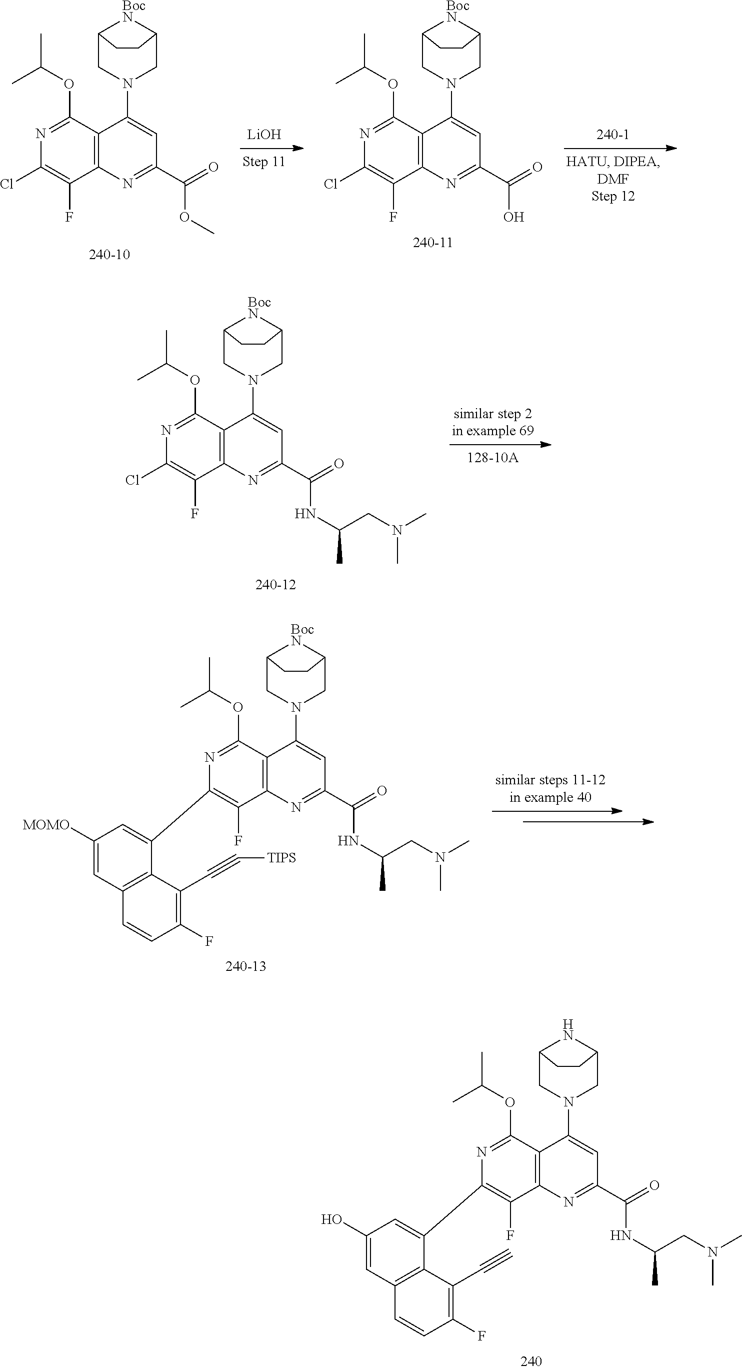

Step 1: To a solution of (R)-2-amino-N,N-dimethylpropanamide HCl (1.2 g, 10.33 mmol) in THF (60 mL) was added LiAlH₄ (784.1 mg, 20.66 mmol) at 0° C. The reaction was stirred at 70° C. for 8 hours. The mixture was cooled to 0° C., quenched with water, 1500 sodium hydroxide solution and water. The mixture was filtered, and the filtrate was added conc HCl (1 mL). The filtrate was concentrated to give 240-1.

Step 2: To propan-2-ol (1409.003 mL, 18.403 mol) was added potassium tert-butanolate (29.6 g, 263.787 mmol) at room temperature under N₂, then stirred at room temperature for 1 hour, 2,6-dichloropyridin-4-amine (100 g, 613.459 mmol) was added to the mixture at room temperature. The reaction was stirred at 100° C. for 36 hours under N₂. Then the mixture was cooled to room temperature, potassium tert-butanolate (29.6 g, 263.787 mmol) was added to the mixture at room temperature, the reaction was stirred at 100° C. for 36 hours under N₂. Then the mixture was cooled to room temperature, filtered. And the filtrate was concentrated, diluted with ethyl acetate. The organic layer was then washed with H₂O and brine, dried over sodium sulfate and concentrated. The residue was triturated with methyl tert-butyl ether and petroleum ether, filtered and the solid was dried to give 240-2.

Step 3: The solution of 240-2 (65 g, 348.264 mmol) in acetic anhydride (294.378 mL, 3134.376 mmol) was stirred at 90° C. for 1.5 hours. The mixture was cooled to room temperature, then filtered. The filter cake was dissolved with ethyl acetate and aq NaHCO₃. The organic layer was dried over sodium sulfate and concentrated to give 240-3.

Step 4: To the solution of 240-3 (20 g, 87.458 mmol) in DMF (200 mL) was added selectfluor (43.4 g, 122.442 mmol) at room temperature under N₂, then stirred at 100° C. for 9 hours. Then the mixture was diluted with ethyl acetate. The organic layer was then washed with H₂O and brine, dried over sodium sulfate and concentrated. The residue was purified by column chromatography on silica gel (petroleum ether to petroleum ether/ethyl acetate=1/4) to give 240-4.

Step 5: To the solution of 240-4 (6 g, 24.324 mmol) in methanol (80 mL) was added NaOH (2.9 g, 72.972 mmol) in H₂O (36.5 mL) at room temperature under N₂, then stirred at room temperature for 16 hours. Then the mixture was diluted with ethyl acetate. The organic layer was then washed with H₂O and brine, dried over sodium sulfate and concentrated to 240-5.

Step 6: To the solution of 240-5 (5.5 g, 26.87 mmol) in EtOH (70 mL) was added silver sulfate (8.38 mg, 26.87 mmol) and diiodine (7.5 mg, 29.56 mmol) at room temperature under N₂, then stirred at room temperature for 2 hours. Then the suspension was filtered, the filtrate was concentrated and diluted with ethyl acetate. The organic layer was then washed with Na₂S₂O₃ and aq NaHCO₃, dried over sodium sulfate and concentrated, the residue was purified by column chromatography on silica gel (petroleum ether to petroleum ether/ethyl acetate=1/4) to give 240-6.

Step 7: To the solution of 240-6 (2.8 g, 8.47 mmol) in Toluene (60 mL) was added tributyl(1-ethoxyvinyl)stannane (3.67 g, 10.16 mmol), Pd(PPh₃)₂Cl₂ (988.7 mg, 1.27 mmol), then stirred at 100° C. for 10 hours under N₂. Then the mixture was cooled to room temperature, diluted with ethyl acetate. The organic layer was then washed with H₂O and brine, dried over sodium sulfate and concentrated to give 240-7, which was used in the next step directly.

Step 8: To the solution of 240-7 (4 g, 8.74 mmol) in THF (50 mL) was added 1N HCl (22 mL) at room temperature under N, then stirred at room temperature for 16 hours. Then the mixture was adjusted with aq NaHCO₃ to pH=8, diluted with ethyl acetate. The organic layer was dried over sodium sulfate and concentrated. The residue was purified by column chromatography on silica gel (petroleum ether to petroleum ether/ethyl acetate=1/4) to give 240-8.

Step 9: To the solution of 240-8 (1.3 g, 5.27 mmol) in methanol (50 mL) was added sodium methanolate (1.4 g, 26.35 mmol) and dimethyl oxalate (2.73 mL, 26.35 mmol) at room temperature under N₂, then stirred at 70° C. for 5 hours under N₂. Then the mixture was cooled to room temperature, filtered and the filter cake was dried to give 240-9.

Step 10: To the solution of 240-9 (640 mg, 2.03 mmol) in DMF (30 mL) was added 1,1,1-trifluoro-N-phenyl-N-((trifluoromethyl)sulfonyl) methanesulfonamide (1017.1 mg, 2.84 mmol), then stirred at room temperature for 2 hours under N₂, then added tert-butyl 3,8-diazabicyclo[3.2.1]octane-8-carboxylate (855.32 mg, 4.029 mmol) at room temperature under N₂, then stirred at room temperature for 2 hours. Then the mixture was diluted with ethyl acetate. The organic layer was then washed with H₂O and brine, dried over sodium sulfate and concentrated. The residue was purified by column chromatography on silica gel (petroleum ether to petroleum ether/ethyl acetate=1/3) to give 240-10.

Step 11: To the solution of 240-10 (400 mg, 0.786 mmol) in THF (50 mL) and H₂O (12.5 mL) was added LiOH (82.4 mg, 1.965 mmol) at 0° C. under N₂, then stirred at room temperature for 2 hours. Then the mixture was adjusted with 1N HCl to pH=4, diluted with ethyl acetate. The organic layer was then washed with H₂O and brine, dried over sodium sulfate and concentrated to give 240-11.

Step 12: To the solution of 240-11 (360 mg, 0.72 mmol) and 240-1 (111.48 mg, 1.09 mmol), in DMF (15 mL) was added DIPEA (375.3 mg, 2.9 mmol) and HATU (553.1 mg, 1.45 mmol) at room temperature under N₂. Then the reaction was stirred at room temperature for 2 hours. The resulting mixture was diluted with ethyl acetate. The organic layer was then washed with H₂O and brine, dried over sodium sulfate and concentrated. The residue was purified by column chromatography on silica gel (DCM to DCM/methanol/NH₄₀H=10/1/0.05) to give 240-12.

Compound 240-13 was prepared from 240-12 and 128-10A following the procedure for the synthesis of compound 217-1 in example 69.

Compound 240 was prepared from 240-13 following the procedure for the synthesis of compound 128 in example 40 as a 3.0 eq of TFA salt. LCMS (ESI, m/z): [M+H]⁺=630.2; ¹H NMR (400 MHz, methanol-d₄, ppm): δ 7.89-7.84 (m, 2H), 7.34-7.31 (m, 2H), 7.29-7.24 (m, 1H), 5.67-5.62 (m, 1H), 4.71-4.70 (m, 1H), 4.24-4.20 (m, 2H), 4.05-3.90 (m, 2H), 3.76-3.67 (m, 2H), 3.51-3.49 (m, 1H), 3.31-3.28 (m, 1H), 3.23-3.17 (m, 1H), 2.99-2.93 (m, 6H), 2.38-2.15 (m, 4H), 1.46-1.43 (m, 6H), 1.37-1.35 (m, 3H). ¹⁹F NMR (376 MHz, methanol-d₄, ppm): δ −111.9 (1F), −148.5 (1F).

### Example 81 Synthesis of Compound 242

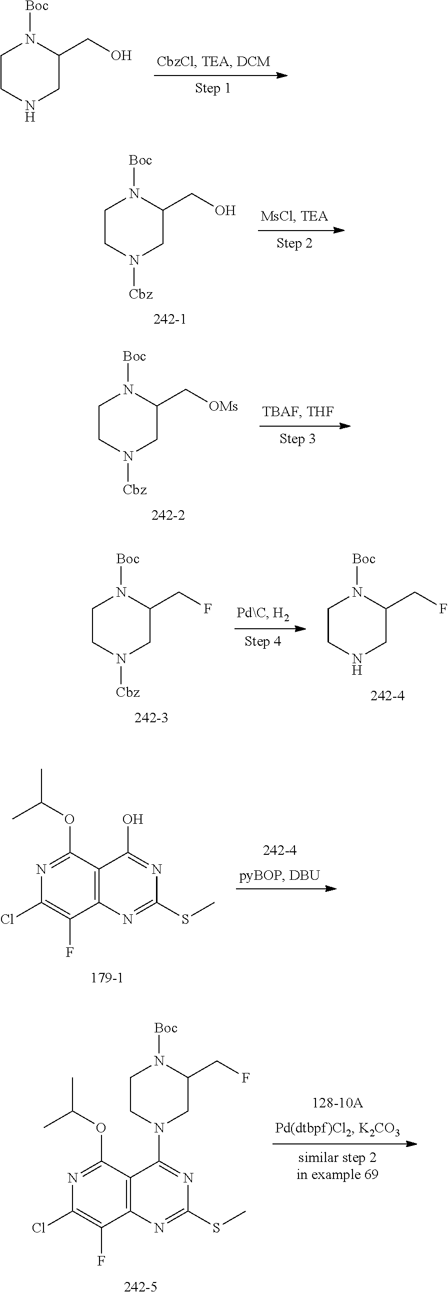

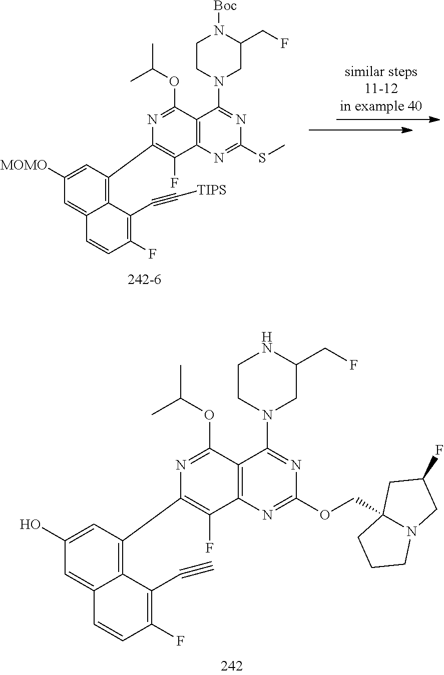

Step 1: To the solution of tert-butyl 2-(hydroxymethyl)piperazine-1-carboxylate (2.16 g, 9.98 mmol) in DCM (50 mL) was added TEA (2.02 g, 19.97 mmol), benzyl chloroformate (1.70 g, 9.98 mmol) was added at 0° C. under N₂, then stirred at room temperature for 3 hours. Then the mixture was diluted with 1 N HCl. The organic layer was then washed with aq NaHCO₃, dried over sodium sulfate and concentrated. The residue was purified by column chromatography on silica gel (petroleum ether to petroleum ether/ethyl acetate=1/1) to give 242-1.

Step 2: To the solution of 242-1 (2.1 g, 5.99 mmol) in DCM (50 mL) was added TEA (1.2 g, 11.98 mmol) and MsCl (1.02 g, 8.98 mmol) at 0° C. under N₂, then stirred at 0° C. for 2 hours. Then the mixture was diluted with H₂O. The organic layer was washed with 1N HCl, aq NaHCO₃, dried over sodium sulfate and concentrated to give 242-2, which was used in the next step directly.

Step 3: To the solution of 242-2 (2.4 g, 5.6 mmol) in THF (30 mL) was added TBAF (28 mL, 28 mmol, 1M in THF) at room temperature under N₂, then stirred at 70° C. for 3 hours. Then the mixture was diluted with ethyl acetate. The organic layer was then washed with H₂O and brine, dried over sodium sulfate and concentrated. The residue was purified by column chromatography on silica gel (petroleum ether to petroleum ether/ethyl acetate=1/4) to give 242-3.

Step 4: To the solution of 242-3 (390 mg, 1.1 mmol) in methanol (30 mL) was added 10% Pd/C (70.7 mg), then stirred at room temperature for 3 hours under H₂. Then the reaction mixture was filtered through celite. The filtrate was concentrated to give 242-4, which was used in the next step directly.

Step 5: To the solution of 179-1 (270 mg, 0.889 mmol), 242-4 (194 mg, 0.889 mmol) in DMF (20 mL) was added PyBOP (693.9 mg, 1.333 mmol) and DBU (406 mg, 2.667 mmol) at room temperature under N₂, then stirred at room temperature for 16 hours. Then the resulting mixture was diluted with ethyl acetate. The organic layer was then washed with H₂O and brine, dried over sodium sulfate and concentrated, the residue was purified by column chromatography on silica gel (petroleum ether to petroleum ether/ethyl acetate=1/4) to give 242-5.

Compound 242-6 was prepared from 242-5 and 128-10A following the procedure for the synthesis of compound 217-1 in example 69.

Compound 242 was prepared from 240-6 following the procedure for the synthesis of compound 128 in example 40 as a 2.0 eq of TFA salt. LCMS (ESI, m/z): [M+H]⁺=665.4; ¹H NMR (400 MHz, methanol-d₄, ppm): δ 7.90-7.84 (m, 1H), 7.37-7.32 (m, 2H), 7.22-7.21 (m, 1H), 5.62-5.53 (m, 2H), 4.89-4.40 (m, 5H), 4.08-3.70 (m, 4H), 3.66-3.51 (m, 3H), 3.50-3.38 (m, 3H), 2.72-2.50 (m, 2H), 2.48-2.32 (m, 3H), 2.25-2.10 (m, 1H), 1.44 (d, J=5.2 Hz, 6H). ¹⁹F NMR (376 MHz, methanol-d₄, ppm): −111.80 (1F), −151.12 (1F), −174.31 (2F).

### Example 82 Synthesis of Compound 244

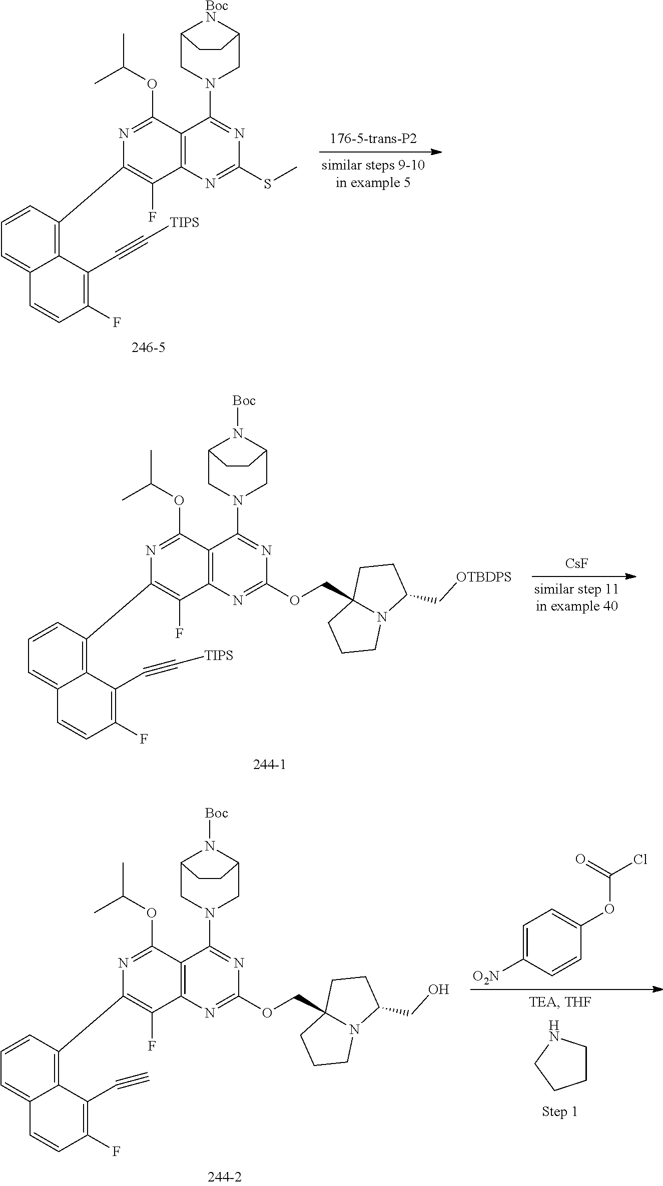

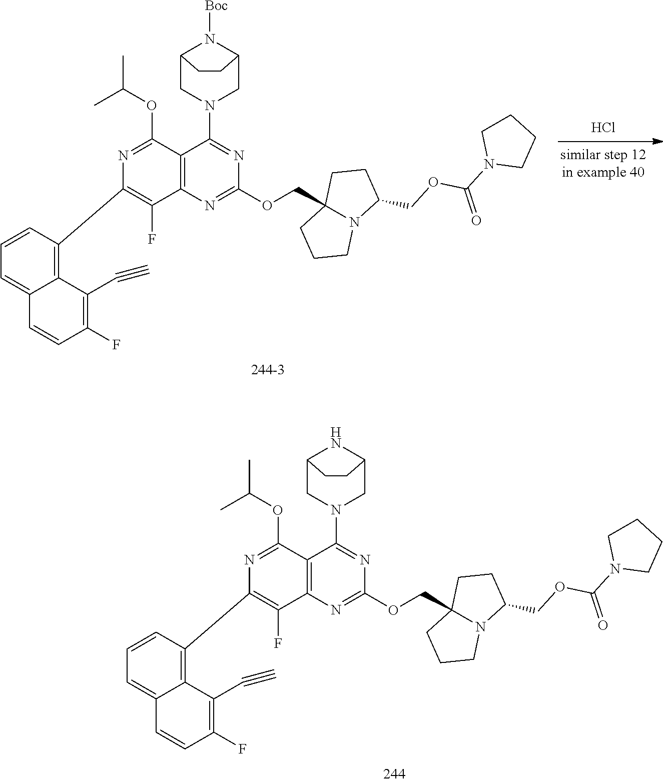 [SMILES: C#Cc1c(F)ccc2cccc(-c3nc(OC(C)C)c4c(N5CC6CCC(C5)N6C(=O)OC(C)(C)C)nc(OC[C@]56CCCN5[C@@H](COC(=O)N5CCCC5)CC6)nc4c3F)c12 ; Cl ; C#Cc1c(F)ccc2cccc(-c3nc(OC(C)C)c4c(N5CC6CCC(C5)N6)nc(OC[C@]56CCCN5[C@@H](COC(=O)N5CCCC5)CC6)nc4c3F)c12]

Compound 244-1 was prepared from 246-5 and 176-5-trans-P2 following the procedure for the synthesis of compound 60-10 in example 5.

Compound 244-2 was prepared from 244-1 following the procedure for the synthesis of compound 128-15 in example 40.

Step 1: To a solution of 244-2 (150 mg, 1.0 mmol) in tetrahydrofuran (10 mL) was added triethylamine (0.5 mL, 7.5 mmol) and 4-nitrophenyl chloroformate (103.0 mg, 3.5 mmol) at room temperature. The mixture was stirred at room temperature overnight under N₂ atmosphere. Then diethylamine (100 mg, 10.0 mmol) in tetrahydrofuran (2.0 mL) was added to the resulting mixture at room temperature. The mixture was stirred for 20 minutes, diluted with ethyl acetate and washed with water, separated. The organic layer was dried over sodium sulfate, filtered and concentrated. The residue was purified by column chromatography on silica gel (dichloromethane to dichloromethane/methanol=0-10%) to give 244-3.

Compound 244 was prepared from 244-3 following the procedure for the synthesis of compound 128 in example 40 as a 3.0 eq of TFA salt. LCMS (ESI, m/z): [M+H]⁺=754.4; ¹H NMR (400 MHz, methanol-d₄, ppm): δ 8.19-8.06 (m, 2H), 7.72-7.60 (m, 2H), 7.51-7.41 (m, 1H), 5.64-5.48 (m, 1H), 4.77-4.69 (m, 3H), 4.68-4.62 (m, 2H), 4.61-4.47 (m, 3H), 4.46-4.36 (m, 1H), 4.34-4.17 (m, 3H), 3.97-3.83 (m, 2H), 3.67-3.57 (m, 1H), 3.51-3.39 (m, 2H), 3.30-3.24 (m, 1H), 2.51-2.33 (m, 2H), 2.32-2.22 (m, 2H), 2.22-1.95 (m, 8H), 1.50-1.37 (m, 6H), 1.23-1.04 (m, 6H). ¹⁹F NMR (376 MHz, methanol-d₄, ppm): δ −107.01 (1F), −151.21 (1F).

Compounds of the present disclosure can be generally prepared by those skilled in the art in view of this disclosure. The compounds in Table 1 were prepared by using similar procedures/methods as shown in the Examples 1-82 herein. Table 1 below also shows some exemplary characterization of representative compounds of the present disclosure prepared herein.

TABLE 1
Exemplary Characterization of representative compounds of the present disclosure

| Compound | | | |
|---|---|---|---|
| No. | Structure | [M + H]⁺ | ¹H-NMR and ¹⁹F-NMR |
| 1 | 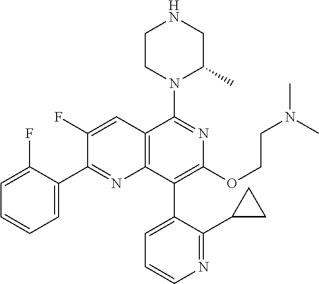 [SMILES: C[C@H]1CNCCN1c1nc(OCCN(C)C)c(-c2cccnc2C2CC2)c2nc(-c3ccccc3F)c(F)cc12] | 545.3 | 3 TFA salt, ¹H NMR (400 MHz, methanol-d₄, ppm): δ 8.49 (d, J = 5.2 Hz, 1H), 8.37 (dd, J = 9.6, 4.0 Hz, 1H), 8.25-8.16 (m, 1H), 7.70-7.62 (m, 1H), 7.55-7.46 (m, 2H), 7.28-7.18 (m, 2H), 4.97-4.90 (m, 1H), 4.80-4.72 (m, 1H), 4.41-4.34 (m, 1H), 3.81-3.70 (m, 2H), 3.67-3.44 (m, 5H), 3.34-3.31 (m, 1H), 2.83 (s, 6H), 2.02-1.91 (m, 1H), 1.40-1.35 (m, 3H), 1.13-0.89 (m, 4H). ¹⁹F NMR (376 MHz, methanol-d₄, ppm): −116.13 (1F), −125.40 (1F). |
| 3 | 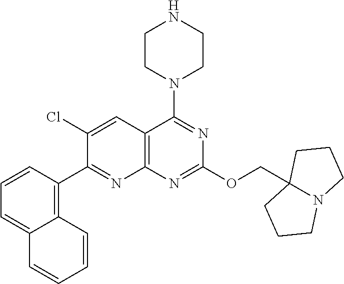 [SMILES: Clc1cc2c(N3CCNCC3)nc(OCC34CCCN3CCC4)nc2nc1-c1cccc2ccccc12] | 515.3 | 3 TFA salt, ¹H NMR (400 MHz, methanol-d₄, ppm): δ 8.69 (s, 1H), 8.04 (d, J = 8.4 Hz, 1H), 7.98 (d, J = 8.4 Hz, 1H), 7.65-7.61 (m, 1H), 7.57-7.54 (m, 2H), 7.54-7.41 (m, 2H), 4.64 (s, 2H), 4.22-4.19 (m, 4H), 3.69-3.59 (m, 2H), 3.50- 3.47 (m, 4H), 3.26-3.23 (m, 2H), 2.32-2.06 (m, 8H). |
| 4 | 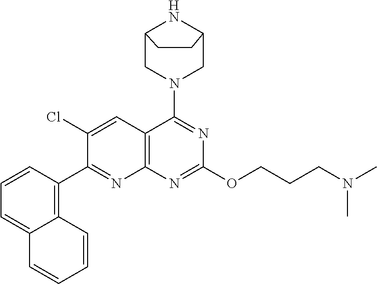 [SMILES: CN(C)CCCOc1nc(N2CC3CCC(C2)N3)c2cc(Cl)c(-c3cccc4ccccc34)nc2n1] | 503.2 | 3 TFA salt, ¹H NMR (400 MHz, methanol-d₄, ppm): δ 8.60 (s, 1H), 8.04 (d, J = 8.4 Hz, 1H), 7.98 (d, J = 8.4 Hz, 1H), 7.65-7.61 (m, 1H), 7.56-7.52 (m, 2H), 7.49-7.43 (m, 2H), 4.77-4.74 (m, 2H), 4.57-4.54 (m, 2H), 4.23 (s, 2H), 3.90 (d, J = 14.0 Hz, 2H), 3.34-3.31 (m, 2H), 2.89 (s, 6H), 2.29-2.21 (m, 2H), 2.12 (m, 4H). |
| 5 | 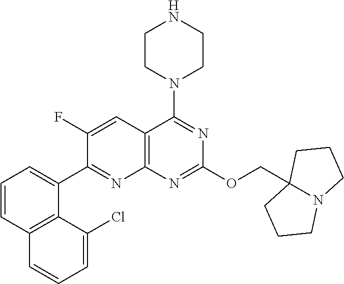 [SMILES: Fc1cc2c(N3CCNCC3)nc(OCC34CCCN3CCC4)nc2nc1-c1cccc2cccc(Cl)c12] | 533.3 | 3 TFA salt, ¹H NMR (400 MHz, methanol-d₄, ppm): δ 8.29 (d, J = 8.4 Hz, 1H), 8.17 (d, J = 8.0 Hz, 1H), 8.03 (d, J = 8.0 Hz, 1H), 7.70 (t, J = 8.0 Hz, 1H), 7.63-7.59 (m, 2H), 7.53 (t, J = 8.0 Hz, 1H), 4.64 (s, 2H), 4.12-4.11 (m, 4H), 3.66-3.63 (m, 2H), 3.50-3.48 (m, 4H), 3.33-3.28 (m, 2H), 2.33-2.28 (m, 2H), 2.24-2.07 (m, 6H). |
| 7 | 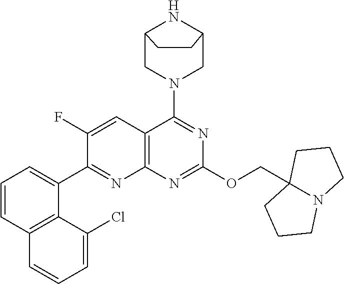 [SMILES: Fc1cc2c(N3CC4CCC(C3)N4)nc(OCC34CCCN3CCC4)nc2nc1-c1cccc2cccc(Cl)c12] | 559.3 | 3 TFA salt, ¹H NMR (400 MHz, methanol-d₄, ppm): δ 8.24 (d, J = 8.8 Hz, 1H), 8.17-8.15 (m, 1H), 8.03-8.01 (m, 1H), 7.71-7.67 (m, 1H), 7.63-7.58 (m, 2H), 7.55-7.51 (m, 1H), 4.73-4.65 (m, 2H), 4.64 (s, 2H), 4.23 (s, 2H), 3.91-3.84 (m, 2H), 3.68-3.62 (m, 2H), 3.32-3.30 (m, 1H), 3.26-3.24 (m, 1H), 2.33-2.28 (m, 2H), 2.24-2.05 (m, 10H). ¹⁹F NMR (376 MHz, methanol-d₄, ppm): δ −124.82 (1F). |
| 8 | 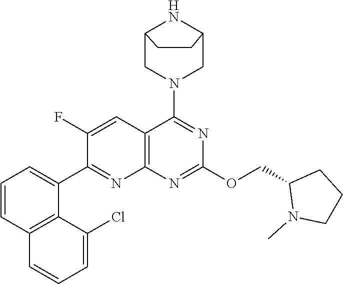 [SMILES: CN1CCC[C@H]1COc1nc(N2CC3CCC(C2)N3)c2cc(F)c(-c3cccc4cccc(Cl)c34)nc2n1] | 533.3 | 3 TFA salt, ¹H NMR (400 MHz, methanol-d₄, ppm): δ 8.23 (d, J = 8.8 Hz, 1H), 8.17-8.15 (m, 1H), 8.02 (d, J = 8.0 Hz, 1H), 7.71-7.67 (m, 1H), 7.63-7.58 (m, 2H), 7.55-7.51 (m, 1H), 4.82-4.81 (m, 1H), 4.70-4.62 (m, 3H), 4.23 (s, 2H), 3.90-3.83 (m, 3H), 3.71 (s, 1H), 3.24-3.21 (m, 1H), 3.05 (s, 3H), 2.40-2.35 (m, 1H), 2.20-2.01 (m, 7H). ¹⁹F NMR (376 MHz, methanol-d₄, ppm): δ −124.93 (1F). |
| 9 | 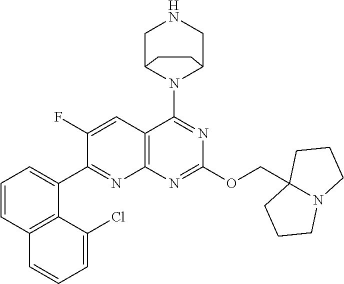 [SMILES: Fc1cc2c(N3C4CCC3CNC4)nc(OCC34CCCN3CCC4)nc2nc1-c1cccc2cccc(Cl)c12] | 559.3 | 3 TFA salt, ¹H NMR (400 MHz, methanol-d₄, ppm): δ 8.33 (d, J = 8.4 Hz, 1H), 8.16 (dd, J = 8.4, 0.8 Hz, 1H), 8.03 (dd, J = 8.4, 0.8 Hz, 1H), 7.70 (t, J = 7.6 Hz, 1H), 7.63-7.59 (m, 2H), 7.53 (t, J = 8.0 Hz, 1H), 5.22-5.19 (m, 2H), 4.64 (s, 2H), 3.71-3.62 (m, 4H), 3.44-3.41 (m, 2H), 3.26-3.24 (m, 2H), 2.35-2.07 (m, 12H). |
| 11 | 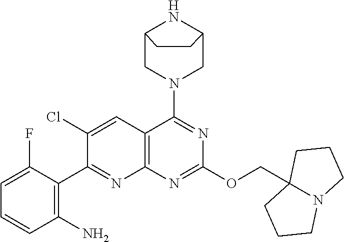 [SMILES: Nc1cccc(F)c1-c1nc2nc(OCC34CCCN3CCC4)nc(N3CC4CCC(C3)N4)c2cc1Cl] | 524.3 | 3 TFA salt, ¹H NMR (400 MHz, methanol-d₄, ppm): δ 8.57 (s, 1H), 7.22-7.16 (m, 1H), 6.65 (d, J = 8.0 Hz, 1H), 6.48- 6.44 (m, 1H), 4.82-4.78 (m, 1H), 4.67-4.59 (m, 3H), 4.19 (d, J = 13.2 Hz, 2H), 3.96 (d, J = 13.6 Hz, 1H), 3.82 (d, J = 14.0 Hz, 1H), 3.68-3.64 (m, 2H), 3.28-3.25 (m, 2H), 2.31-2.06 (m, 12H). |
| 12 | 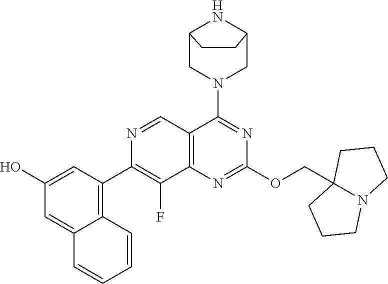 [SMILES: Oc1cc(-c2ncc3c(N4CC5CCC(C4)N5)nc(OCC45CCCN4CCC5)nc3c2F)c2ccccc2c1] | 541.3 | 2 TFA salt, ¹H NMR (400 MHz, DMSO-d₆, ppm): δ 10.38 (brs, 1H), 10.04 (brs, 1H), 9.46-9.44 (m, 1H), 9.28-9.19 (m, 2H), 7.78 (d, J = 8.4 Hz, 1H), 7.50 (d, J = 8.4 Hz, 1H), 7.42 (t, J = 7.2 Hz, 1H), 7.28-7.27 (m, 1H), 7.24-7.20 (m, 2H), 4.70-4.67 (m, 2H), 4.56 (s, 2H), 4.21-4.16 (m, 2H), 3.88- 3.85 (m, 2H), 3.55-3.45 (m, 2H), 3.25-3.15 (m, 2H), 2.19- 1.88 (m, 12H). ¹⁹F NMR (376 MHz, DMSO-d₆, ppm): δ −139.21 (1F). |
| 13 | 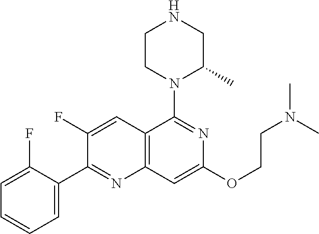 [SMILES: C[C@H]1CNCCN1c1nc(OCCN(C)C)cc2nc(-c3ccccc3F)c(F)cc12] | 428.2 | 3 TFA salt, ¹H NMR (400 MHz, methanol-d₄, ppm): δ 8.35 (d, J = 0.80 Hz, 1H), 7.70-7.65 (m, 1H), 7.60-7.58 (m, 1H), 7.38 (t, J = 7.4 Hz, 1H), 7.28 (t, J = 9.6 Hz, 1H), 7.13 (s, 1H), 4.78-4.72 (m, 2H), 4.18-4.17 (brs, 1H), 3.67-3.63 (m, 3H), 3.60-3.44 (m, 4H), 3.30-3.22 (m, 1H), 3.01 (s, 6H), 1.23 (d, J = 6.4 Hz, 3H). |
| 14 | 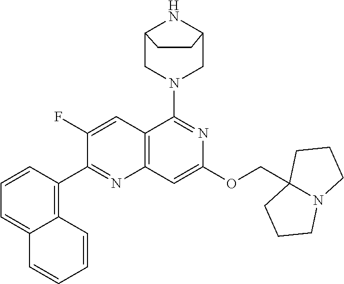 [SMILES: Fc1cc2c(N3CC4CCC(C3)N4)nc(OCC34CCCN3CCC4)cc2nc1-c1cccc2ccccc12] | 524.3 | 3 TFA salt, ¹H NMR (400 MHz, methanol-d₄, ppm): δ 8.28 (d, J = 9.6 Hz, 1H), 8.09-8.06 (m, 1H), 8.00 (d, J = 8.0 Hz, 1H), 7.68-7.62 (m, 3H), 7.57-7.53 (m, 1H), 7.49-7.45 (m, 1H), 6.98 (s, 1H), 4.58 (s, 2H), 4.23 (brs, 2H), 4.06-4.09 (m, 2H), 3.67-3.62 (m, 4H), 3.33-3.27 (m, 2H), 2.43-2.07 (m, 12H). |
| 15 | 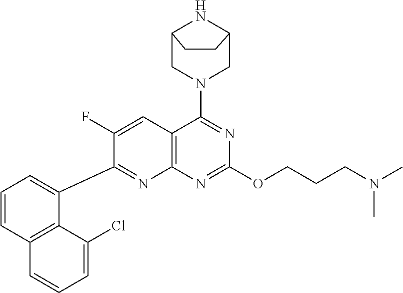 [SMILES: CN(C)CCCOc1nc(N2CC3CCC(C2)N3)c2cc(F)c(-c3cccc4cccc(Cl)c34)nc2n1] | 521.3 | 3 TFA salt, ¹H NMR (400 MHz, methanol-d₄, ppm): δ 8.21- 8.15 (m, 2H), 8.02 (d, J = 7.6 Hz, 1H), 7.69 (t, J = 7.6 Hz, 1H), 7.62-7.58 (m, 2H), 7.52 (t, J = 8.0 Hz, 1H), 4.67-4.54 (m, 4H), 4.23 (s, 2H), 3.84 (t, J = 13.2 Hz, 2H), 3.37-3.33 (m, 2H), 2.95-2.85 (m, 6H), 2.27-2.15 (m, 6H). |
| 16 | 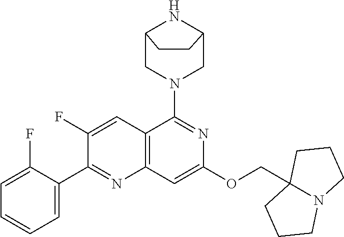 [SMILES: Fc1ccccc1-c1nc2cc(OCC34CCCN3CCC4)nc(N3CC4CCC(C3)N4)c2cc1F] | 492.3 | 3 TFA salt, ¹H NMR (400 MHz, methanol-d₄, ppm): δ 8.18 (d, J = 9.6 Hz, 1H), 7.69-7.65 (m, 1H), 7.61-7.58 (m, 1H), 7.38 (td, J = 7.6 Hz, 0.8 Hz, 1H), 7.31-7.26 (m, 1H), 6.99 (s, 1H), 4.56 (s, 2H), 4.21 (brs, 3H), 4.04-4.01 (m, 2H), 3.70- 3.60 (m, 4H), 3.33-3.27 (m, 1H), 2.39-2.35 (m, 2H), 2.34- 2.30 (m, 2H), 2.29-2.20 (m, 6H), 2.19-2.07 (m, 2H). ¹⁹F NMR (376 MHz, methanol-d₄, ppm): δ −116.37 (1F), −126.12 (1F). |
| 19 | 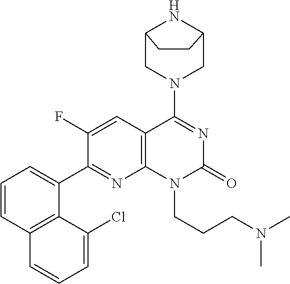 [SMILES: CN(C)CCCn1c(=O)nc(N2CC3CCC(C2)N3)c2cc(F)c(-c3cccc4cccc(Cl)c34)nc21] | 521.3 | 3 TFA salt, ¹H NMR (400 MHz, methanol-d₄, ppm): δ 8.62 (s, 1H), 7.40 (d, J = 8.4 Hz, 1H), 7.43-7.39 (m, 1H), 7.33 (d, J = 8.0 Hz, 1H), 7.28 (d, J = 2.4 Hz, 1H), 7.22-7.13 (m, 2H), 4.79-4.78 (m, 2H), 4.63 (s, 2H), 4.23 (s, 2H), 3.92 (d, J = 14.0 Hz, 2H), 3.25-3.24 (m, 2H), 2.32-2.06 (m, 12H). |
| 21 | 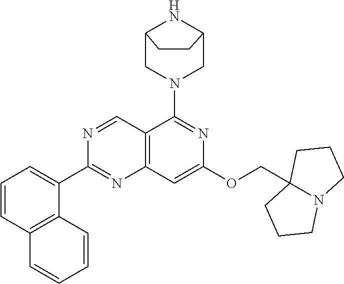 [SMILES: c1ccc2c(-c3ncc4c(N5CC6CCC(C5)N6)nc(OCC56CCCN5CCC6)cc4n3)cccc2c1] | 507.4 | 3 TFA salt, ¹H NMR (400 MHz, DMSO-d₆, ppm): δ 10.36 (s, 1H), 9.56 (s, 1H), 9.37-9.29 (m, 1H), 9.21-9.12 (m, 1H), 8.82-8.79 (m, 1H), 8.17 (dd, J = 7.2, 1.2 Hz, 1H), 8.10 (d, J = 8.0 Hz, 1H), 8.03-8.01 (m, 1H), 7.66 (t, J = 8.0 Hz, 1H), 7.57-7.55 (m, 2H), 6.67 (s, 1H), 4.48 (s, 2H), 4.42 (d, J = 13.2 Hz, 2H), 4.14 (s, 2H), 3.77 (d, J = 13.6 Hz, 2H), 3.53- 3.49 (m, 2H), 3.22-3.19 (m, 2H), 2.16-1.97 (m, 12H). |
| 22 | 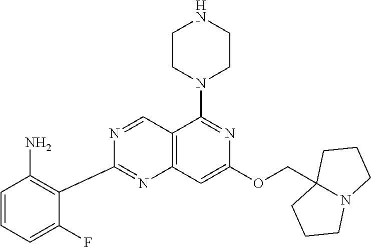 [SMILES: Nc1cccc(F)c1-c1ncc2c(N3CCNCC3)nc(OCC34CCCN3CCC4)cc2n1] | 464.4 | 3 TFA salt, ¹H NMR (400 MHz, DMSO-d₆, ppm): δ 10.34 (s, 1H), 9.53 (s, 1H), 9.08 (s, 2H), 7.16-7.10 (m, 1H), 6.69 (s, 1H), 6.61 (d, J = 8.4 Hz, 1H), 6.40-6.36 (m, 1H), 4.48 (S, 2H), 3.84-3.80 (m, 4H), 3.52-3.47 (m, 2H), 3.34-3.30 (m, 4H), 3.21-3.18 (m, 2H), 2.14-1.94 (m, 8H). |
| 23 | 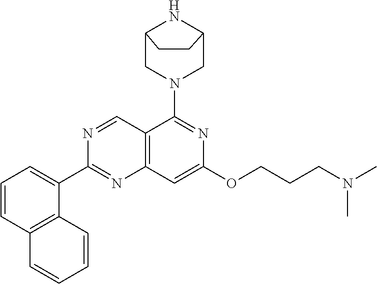 [SMILES: CN(C)CCCOc1cc2nc(-c3cccc4ccccc34)ncc2c(N2CC3CCC(C2)N3)n1] | 469.4 | 3 TFA salt, ¹H NMR (400 MHz, DMSO-d₆, ppm): δ 9.60 (s, 1H), 9.54 (s, 1H), 9.25-9.23 (m, 1H), 9.09 (s, 1H), 8.76-8.73 (m, 1H), 8.15-8.08 (m, 2H), 8.03-8.00 (m, 1H), 7.65 (t, J = 8.0 Hz, 1H), 7.58-7.52 (m, 2H), 6.57 (s, 1H), 4.39-4.36 (m, 4H), 4.14 (s, 2H), 3.74 (d, J = 13.6 Hz, 2H), 3.24-3.19 (m, 2H), 2.80 (s, 3H), 2.79 (s, 3H), 2.15-2.08 (m, 2H), 2.02-1.93 (m, 4H). |
| 24 | 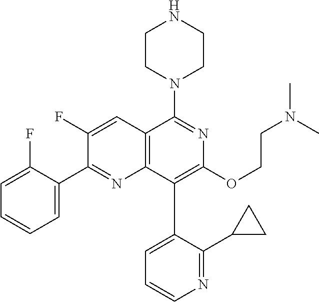 [SMILES: CN(C)CCOc1nc(N2CCNCC2)c2cc(F)c(-c3ccccc3F)nc2c1-c1cccnc1C1CC1] | 531.3 | 3 TFA salt, ¹H NMR (400 MHz, methanol-d₄, ppm): δ 8.54 (d, J = 6.0 Hz, 1H), 8.37-8.37 (m, 2H), 7.79-7.75 (m, 1H), 7.53-7.46 (m, 2H), 7.27-7.18 (m, 2H), 4.94-4.91 (m, 1H), 4.80-4.78 (m, 1H), 3.88-3.86 (m, 4H), 3.60-3.55 (m, 6H), 2.85 (s, 6H), 2.10-2.00 (m, 1H), 1.13-0.99 (m, 4H). |
| 25 | 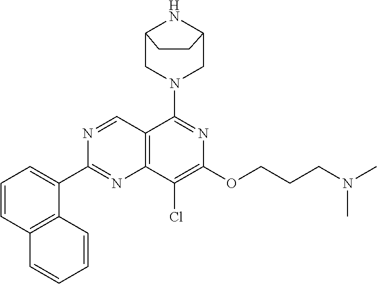 [SMILES: CN(C)CCCOc1nc(N2CC3CCC(C2)N3)c2cnc(-c3cccc4ccccc34)nc2c1Cl] | 503.3 | 3 TFA salt, ¹H NMR (400 MHz, DMSO-d₆, ppm): δ 9.57- 9.47 (m, 2H), 9.24 (s, 1H), 9.08-9.06 (m, 2H), 8.33-8.31 (m, 1H), 8.13 (d, J = 8.0 Hz, 1H), 8.05-8.02 (m, 1H), 7.70-7.66 (m, 1H), 7.60-7.55 (m, 2H), 4.51 (t, J = 5.6 Hz, 2H), 4.43 (d, J = 13.6 Hz, 2H), 4.13 (s, 2H), 3.81 (d, J = 13.6 Hz, 2H), 3.25-3.20 (m, 2H), 2.82 (s, 3H), 2.81 (s, 3H), 2.17-2.13 (m, 2H), 1.99-1.91 (m, 4H). |
| 26 | 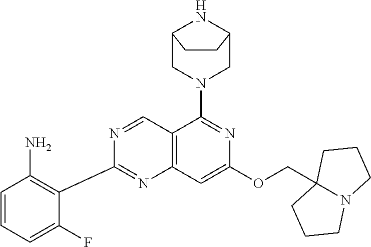 [SMILES: Nc1cccc(F)c1-c1ncc2c(N3CC4CCC(C3)N4)nc(OCC34CCCN3CCC4)cc2n1] | 490.4 | 3 TFA salt, ¹H NMR (400 MHz, DMSO-d₆, ppm): δ 10.42 (s, 1H), 9.47 (s, 1H), 9.38-9.32 (m, 1H), 9.25-9.15 (m, 1H), 7.19-7.13 (m, 1H), 6.64-6.63 (m, 2H), 6.41 (t, J = 8.8 Hz, 1H), 4.49 (s, 2H), 4.39 (d, J = 13.6 Hz, 2H), 4.16 (s, 2H), 3.76 (d, J = 13.6 Hz, 2H), 3.57-3.49 (m, 2H), 3.26-3.20 (m, 2H), 2.18-1.98 (m, 12H). ¹⁹F NMR (376 MHz, DMSO-d₆, ppm): δ −113.28 (1F). |
| 27 | 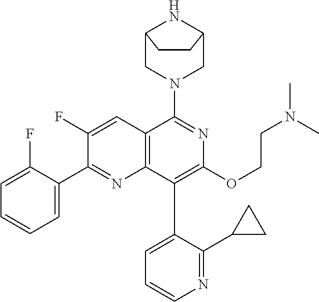 [SMILES: CN(C)CCOc1nc(N2CC3CCC(C2)N3)c2cc(F)c(-c3ccccc3F)nc2c1-c1cccnc1C1CC1] | 557.4 | 4 TFA salt, ¹H NMR (400 MHz, methanol-d₄, ppm): δ 8.43 (brs, 1H), 8.24 (d, J = 10.0 Hz, 1H), 7.96 (d, J = 7.6 Hz, 1H), 7.52-7.47 (m, 3H), 7.26-7.17 (m, 2H), 4.25 (brs, 2H), 4.17- 4.13 (m, 2H), 3.72-3.68 (m, 2H), 3.56-3.54 (m, 2H), 2.81 (s, 6H), 2.43-2.40 (m, 2H), 2.24-2.15 (m, 2H), 2.03-1.98 (m, 1H), 1.84-1.80 (m, 1H), 1.60-1.56 (m, 1H), 1.10-0.97 (m, 2H), 0.93-0.80 (m, 2H). ¹⁹F NMR (376 MHz, methanol-d₄, ppm): δ −116.11 (1F), −126.05 (1F). |
| 28 | 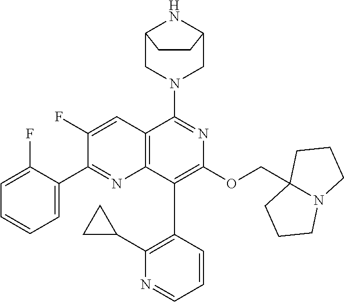 [SMILES: Fc1ccccc1-c1nc2c(-c3cccnc3C3CC3)c(OCC34CCCN3CCC4)nc(N3CC4CCC(C3)N4)c2cc1F] | 609.5 | 4 TFA salt, ¹H NMR (400 MHz, methanol-d₄, ppm): δ 8.46- 8.44 (m, 1H), 8.24 (d, J = 10 Hz, 1H), 7.98 (d, J = 6.4 Hz, 1H), 7.53-7.47 (m, 3H), 7.26-7.18 (m, 2H), 4.60 (s, 2H), 4.25-4.15 (m, 4H), 3.70 (dd, J = 13.6, 2.8 Hz, 2H), 3.40-3.30 (m, 1H), 3.13-3.02 (m, 3H), 2.41-2.39 (m, 2H), 2.24-1.86 (m, 10H), 1.75-1.70 (m, 1H), 1.12-1.05 (m, 1H), 1.00-0.80 (m, 3H). ¹⁹F NMR (376 MHz, methanol-d₄, ppm): δ −116.11 (1F), −126.22 (1F). |
| 29 | 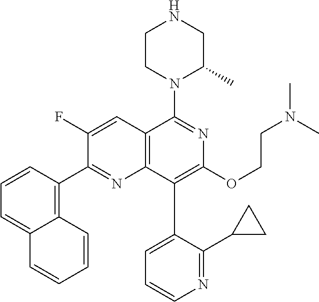 [SMILES: C[C@H]1CNCCN1c1nc(OCCN(C)C)c(-c2cccnc2C2CC2)c2nc(-c3cccc4ccccc34)c(F)cc12] | 577.4 | 3 TFA salt, ¹H NMR (400 MHz, DMSO-d₆, ppm): δ 9.82 (s, 1H), 9.21 (s, 1H), 8.91 (s, 1H), 8.50 (d, J = 10 Hz, 1H), 8.29- 8.27 (m, 1H), 8.07-8.04 (m, 1H), 7.99 (d, J = 8.0 Hz, 1H), 7.74-7.70 (m, 1H), 7.61-7.51 (m, 4H), 7.47-7.42 (m, 1H), 7.13-7.09 (m, 1H), 4.68-4.64 (m, 2H), 4.31-4.28 (m, 1H), 3.50-3.41 (m, 4H), 3.39-3.32 (m, 2H), 3.30-3.10 (m, 2H), 2.68-2.63 (m, 6H), 1.68-1.61 (m, 1H), 1.31-1.29 (m, 3H), 0.92-0.84 (m, 2H), 0.77-0.68 (m, 2H). |
| 30 | 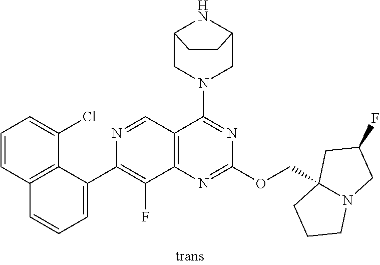 [SMILES: Fc1c(-c2cccc3cccc(Cl)c23)ncc2c(N3CC4CCC(C3)N4)nc(OC[C@@]34CCCN3C[C@H](F)C4)nc12] | 577.3 | 3 TFA salt, ¹H NMR (400 MHz, methanol-d₄, ppm): δ 9.12 (s, 1H), 8.15-8.13 (m, 1H), 8.02-8.00 (m, 1H), 7.68 (t, J = 7.2 Hz, 1H), 7.60-7.57 (m, 2H), 7.51 (t, J = 8.0 Hz, 1H), 5.65-5.47 (m, 1H), 4.92-4.86 (m, 2H), 4.71-4.67 (m, 2H), 4.26 (s, 2H), 4.04-3.82 (m, 5H), 3.50-3.40 (m, 1H), 2.77- 2.51 (m, 2H), 2.46-2.28 (m, 3H), 2.22-2.04 (m, 5H). ¹⁹F NMR (376 MHz, methanol-d₄, ppm): δ −139.36 (m, 1F), −174.23 (m, 1F). |
| 31 | 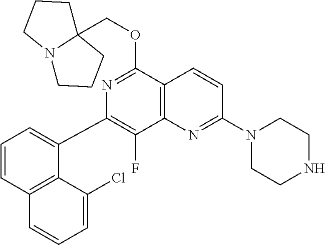 [SMILES: Fc1c(-c2cccc3cccc(Cl)c23)nc(OCC23CCCN2CCC3)c2ccc(N3CCNCC3)nc12] | 532.3 | 3 TFA salt, ¹H NMR (400 MHz, DMSO-d₆, ppm): δ 10.55 (brs, 1H), 9.19 (brs, 2H), 8.38-8.29 (m, 1H), 8.15-8.10 (m, 1H), 8.08-7.98 (m, 1H), 7.71-7.65 (m, 1H), 7.63-7.58 (m, 2H), 7.56-7.50 (m, 1H), 7.44-7.36 (m, 1H), 4.53-4.44 (m, 1H), 4.43-4.38 (m, 1H), 4.04-3.98 (m, 4H), 3.28-3.18 (m, 6H), 2.05 (m, 10H). ¹⁹F NMR (376 MHz, DMSO-d₆, ppm): δ −148.61 (1F). |
| 32 | 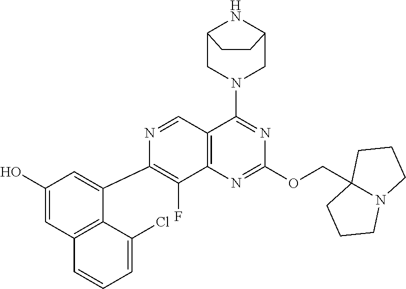 [SMILES: Oc1cc(-c2ncc3c(N4CC5CCC(C4)N5)nc(OCC45CCCN4CCC5)nc3c2F)c2c(Cl)cccc2c1] | 575.3 | 3 TFA salt, ¹H NMR (400 MHz, methanol-d₄, ppm): δ 9.10 (s, 1H), 7.75 (dd, J = 8.0, 1.2 Hz, 1H), 7.38-7.31 (m, 3H), 7.14-7.13 (m, 1H), 4.87-4.83 (m, 2H), 4.67 (s, 2H), 4.28- 4.22 (m, 2H), 3.99-3.92 (m, 2H), 3.71-3.65 (m, 2H), 3.31- 3.25 (m, 2H), 2.36-2.29 (m, 2H), 2.25-2.06 (m, 10H). ¹⁹F NMR (376 MHz, methanol-d₄, ppm): δ −139.24 (1F). |
| 33 | 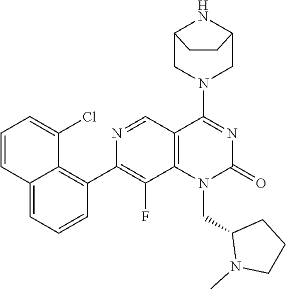 [SMILES: CN1CCC[C@H]1Cn1c(=O)nc(N2CC3CCC(C2)N3)c2cnc(-c3cccc4cccc(Cl)c34)c(F)c21] | 533.3 | 3 TFA salt, ¹H NMR (400 MHz, methanol-d₄, ppm): δ 8.88 (s, 1H), 8.15 (d, J = 7.6 Hz, 1H), 8.02 (d, J = 8.0 Hz, 1H), 7.69 (t, J = 7.6 Hz, 1H), 7.64-7.59 (m, 2H), 7.52 (t, J = 8.0 Hz, 1H), 4.86-4.82 (m, 1H), 4.67-4.64 (m, 2H), 4.59-4.45 (m, 1H), 4.23 (s, 2H), 4.01-3.70 (m, 4H), 3.24-3.14 (m, 1H), 2.96-2.94 (m, 3H), 2.35-1.83 (m, 8H). ¹⁹F NMR (376 MHz, methanol-d₄, ppm): δ −134.76 (1F). |
| 34 | 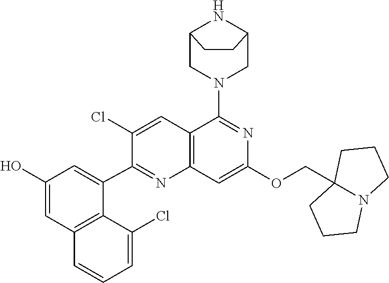 [SMILES: Oc1cc(-c2nc3cc(OCC45CCCN4CCC5)nc(N4CC5CCC(C4)N5)c3cc2Cl)c2c(Cl)cccc2c1] | 590.3 | 3 TFA salt, ¹H NMR (400 MHz, methanol-d₄, ppm): δ 8.42 (s, 1H), 7.75 (d, J = 7.2 Hz, 1H), 7.38-7.29 (m, 3H), 7.03 (d, J = 2.4 Hz, 1H), 6.87 (s, 1H), 4.61-4.52 (m, 2H), 4.22 (s, 2H), 4.11-4.03 (m, 2H), 3.71-3.61 (m, 4H), 3.35-3.25 (m, 2H), 2.41-2.06 (m, 12H). |
| 36 | 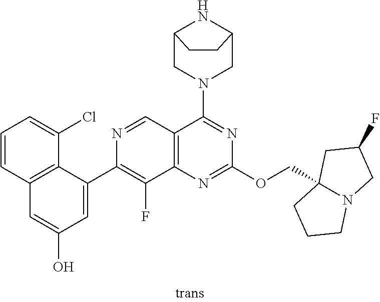 [SMILES: Oc1cc(-c2ncc3c(N4CC5CCC(C4)N5)nc(OC[C@@]45CCCN4C[C@H](F)C5)nc3c2F)c2c(Cl)cccc2c1] | 593.3 | 3 TFA salt, ¹H NMR (400 MHz, methanol-d₄, ppm): δ 9.10 (s, 1H), 7.75 (dd, J = 8.0, 1.2 Hz, 1H), 7.39-7.31 (m, 3H), 7.13-7.12 (m, 1H), 5.65-5.48 (m, 1H), 4.92-4.83 (m, 2H), 4.73-4.66 (m, 2H), 4.29-4.22 (m, 2H), 4.04-3.83 (m, 5H), 3.49-3.42 (m, 1H), 2.76-2.53 (m, 2H), 2.45-2.29 (m, 3H), 2.23-2.04 (m, 5H). ¹⁹F NMR (376 MHz, methanol-d₄, ppm): δ −139.34 (1F), −174.21 (1F). |
| 37 | 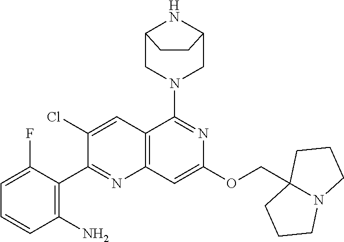 [SMILES: Nc1cccc(F)c1-c1nc2cc(OCC34CCCN3CCC4)nc(N3CC4CCC(C3)N4)c2cc1Cl] | 523.3 | 2.4 TFA salt, ¹H NMR (400 MHz, methanol-d₄, ppm): δ 8.50 (s, 1H), 7.22-7.16 (m, 1H), 6.89 (s, 1H), 6.66 (d, J = 8.4 Hz, 1H), 6.47 (t, J = 8.8 Hz, 1H), 4.56 (s, 2H), 4.20-4.05 (m, 4H), 3.72-3.61 (m, 4H), 3.33-3.26 (m, 2H), 2.38-2.06 (m, 12H). ¹⁹F NMR (376 MHz, methanol-d₄, ppm): δ −117.10 (1F). |
| 39 | 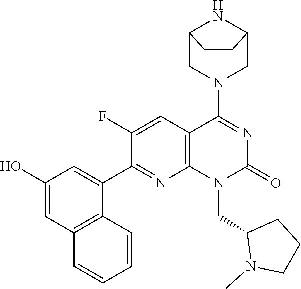 [SMILES: CN1CCC[C@H]1Cn1c(=O)nc(N2CC3CCC(C2)N3)c2cc(F)c(-c3cc(O)cc4ccccc34)nc21] | 515.3 | 2 TFA salt, ¹H NMR (400 MHz, methanol-d₄, ppm): δ 8.33- 8.30 (m, 1H), 7.77-7.75 (m, 1H), 7.68-7.66 (m, 1H), 7.46- 7.43 (m, 1H), 7.30-7.28 (m, 3H), 4.73-4.67 (m, 4H), 4.25- 4.20 (m, 2H), 3.89-3.85 (m, 2H), 3.80-3.70 (m, 1H), 3.69- 3.58 (m, 1H), 3.11-3.04 (m, 1H), 2.86 (s, 3H), 2.24-1.92 (m, 8H). |
| 40 | 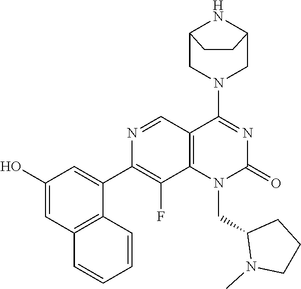 [SMILES: CN1CCC[C@H]1Cn1c(=O)nc(N2CC3CCC(C2)N3)c2cnc(-c3cc(O)cc4ccccc34)c(F)c21] | 515.3 | 2 TFA salt, ¹H NMR (400 MHz, methanol-d₄, ppm): δ 8.92 (s, 1H), 7.74 (d, J = 8.4 Hz, 1H), 7.57 (d, J = 8.4 Hz, 1H), 7.44-7.40 (m, 1H), 7.29-7.23 (m, 3H), 4.98-4.92 (m, 1H), 4.71-4.63 (m, 2H), 4.55-4.51 (m, 1H), 4.23-4.19 (m, 2H), 4.03-3.99 (m, 1H), 3.85-3.73 (m, 3H), 3.23-3.16 (m, 1H), 2.94 (s, 3H), 2.38-2.25 (m, 1H), 2.20-1.99 (m, 6H), 1.97- 1.85 (m, 1H). ¹⁹F NMR (376 MHz, methanol-d₄, ppm): δ −135.41 (1F). |
| 41 | 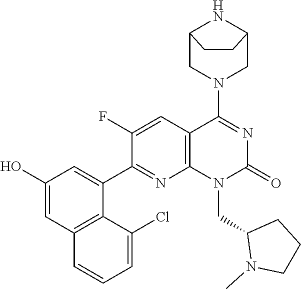 [SMILES: CN1CCC[C@H]1Cn1c(=O)nc(N2CC3CCC(C2)N3)c2cc(F)c(-c3cc(O)cc4cccc(Cl)c34)nc21] | 549.3 | 2 TFA salt, ¹H NMR (400 MHz, methanol-d₄, ppm): δ 8.20- 8.18 (m, 1H), 7.78-7.76 (m, 1H), 7.40-7.35 (m, 3H), 7.18- 7.17 (m, 1H), 4.73-4.67 (m, 4H), 4.25-4.20 (m, 2H), 3.90- 3.60 (m, 4H), 3.10-3.07 (m, 1H), 2.91 (d, J = 22.8 Hz, 3H), 2.19-1.92(m, 8H). |
| 42 | 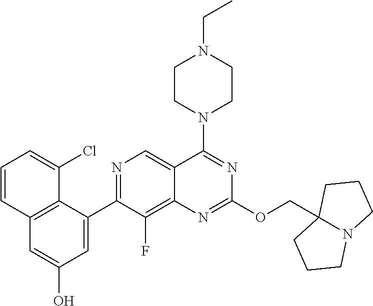 [SMILES: CCN1CCN(c2nc(OCC34CCCN3CCC4)nc3c(F)c(-c4cc(O)cc5cccc(Cl)c45)ncc23)CC1] | 577.3 | 3 TFA salt, ¹H NMR (400 MHz, methanol-d₄, ppm): δ 9.13 (s, 1H), 7.75 (dd, J = 8.0, 1.2 Hz, 1H), 7.38-7.30 (m, 3H), 7.14-7.13 (m, 1H), 4.68 (s, 2H), 4.32-3.29 (m, 10H), 3.28- 3.27 (m, 4H), 2.40-2.05 (m, 8H), 1.40 (t, J = 7.6 Hz, 3H). |
| 46 | 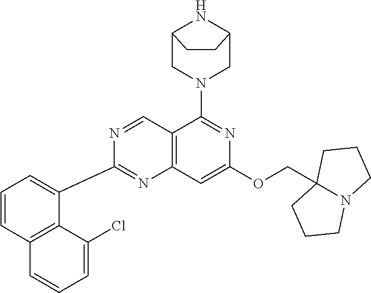 [SMILES: Clc1cccc2cccc(-c3ncc4c(N5CC6CCC(C5)N6)nc(OCC56CCCN5CCC6)cc4n3)c12] | 541.3 | 3 TFA salt, ¹H NMR (400 MHz, methanol-d₄, ppm): δ 9.43 (s, 1H), 8.15-8.12 (m, 1H), 8.02-8.00 (m, 1H), 7.70-7.65 (m, 2H), 7.61-7.58 (m, 1H), 7.53-7.50 (m, 1H), 6.68 (s, 1H), 4.61-4.53 (m, 3H), 4.45-4.42 (m, 1H), 4.23 (s, 2H), 3.88- 3.80 (m, 2H), 3.68-3.62 (m, 2H), 3.37-3.34 (m, 2H), 2.33- 2.08 (m, 12H). |
| 48 | 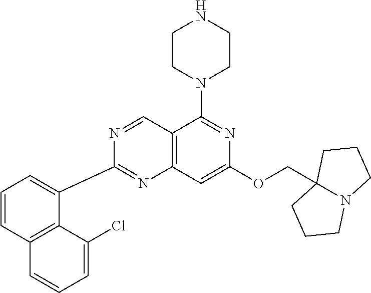 [SMILES: Clc1cccc2cccc(-c3ncc4c(N5CCNCC5)nc(OCC56CCCN5CCC6)cc4n3)c12] | 515.3 | 3 TFA salt, ¹H NMR (400 MHz, methanol-d₄, ppm): δ 9.49 (s, 1H), 8.13 (dd, J = 7.2, 2.4 Hz, 1H), 8.01 (dd, J = 8.0,0.8 Hz, 1H), 7.70-7.65 (m, 2H), 7.60-7.58 (m, 1H), 7.53-7.49 (m, 1H), 6.74 (s, 1H), 4.64 (d, J = 4.4 Hz, 2H), 4.02-3.99 (m, 4H),3.69-3.63 (m, 2H), 3.53-3.49 (m, 4H), 3.33-3.31 (m, 2H), 2.34-2.08 (m, 8H). |
| 49 | 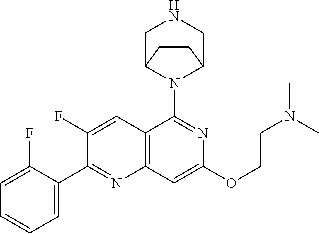 [SMILES: CN(C)CCOc1cc2nc(-c3ccccc3F)c(F)cc2c(N2C3CCC2CNC3)n1] | 440.3 | 3 TFA salt, ¹H NMR (400 MHz, methanol-d₄, ppm): δ 8.30 (d, J = 10.0 Hz, 1H), 7.69-7.65 (m, 1H), 7.63-7.57 (m, 1H), 7.40-7.36 (m, 1H), 7.31-7.26 (m, 1H), 6.98 (s, 1H), 4.74- 4.71 (m, 4H), 3.72-3.69 (m, 2H), 3.64-3.61 (m, 2H), 3.40- 3.37 (m, 2H), 3.00 (s, 6H), 2.28-2.26 (m, 2H), 2.07-2.05 (m, 2H). |
| 51 | 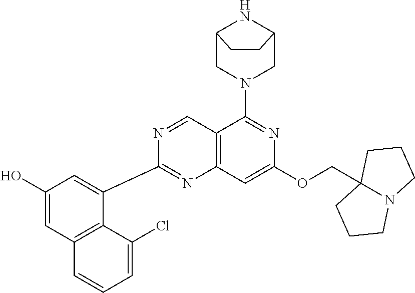 [SMILES: Oc1cc(-c2ncc3c(N4CC5CCC(C4)N5)nc(OCC45CCCN4CCC5)cc3n2)c2c(Cl)cccc2c1] | 557.3 | 3 TFA salt, ¹H NMR (400 MHz, methanol-d₄, ppm): δ 9.42 (s, 1H), 7.74 (d, J = 7.2 Hz, 1H), 7.39-7.32 (m, 3H), 7.24 (d, J = 2.4 Hz, 1H), 6.67 (s, 1H), 4.62 (s, 2H), 4.55-4.52 (m, 1H), 4.44-4.40 (m, 1H), 4.23 (s, 2H), 3.89-3.81 (m, 2H), 3.68-3.62 (m, 2H), 3.37-3.34 (m, 2H), 2.34-2.09 (m, 12H). |
| 52 | 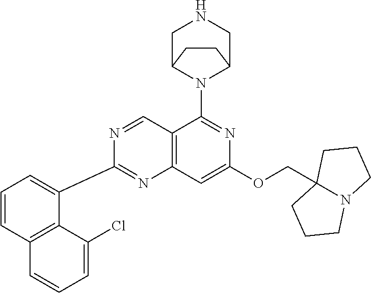 [SMILES: Clc1cccc2cccc(-c3ncc4c(N5C6CCC5CNC6)nc(OCC56CCCN5CCC6)cc4n3)c12] | 541.3 | 3 TFA salt, ¹H NMR (400 MHz, methanol-d₄, ppm): δ 9.53 (d, J = 0.8 Hz,1H), 8.14-8.12 (m, 1H),7.99 (d, J = 0.8 Hz, 1H), 7.69-7.67 (m, 2H), 7.58 (d, J = 1.2 Hz, 1H), 7.52 (d, J = 8.4 Hz, 1H), 6.71 (d, J = 0.8 Hz, 1H), 5.02-4.94 (m, 2H), 4.64-4.50 (m, 2H), 3.70-3.57 (m, 4H), 3.46-3.39 (m, 2H), 3.39-3.30 (m, 2H), 2.35-2.09 (m, 12H). |
| 54 | 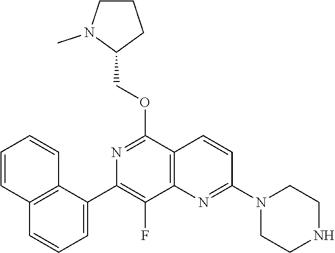 [SMILES: CN1CCC[C@@H]1COc1nc(-c2cccc3ccccc23)c(F)c2nc(N3CCNCC3)ccc12] | 472.3 | 3 TFA salt, ¹H NMR (400 MHz, methanol-d₄, ppm): δ 8.41 (dd, J = 9.6, 1.6 Hz, 1H), 7.99-7.94 (m, 2H), 7.76 (d, J = 8.8 Hz, 1H), 7.64-7.56 (m, 2H), 7.52-7.43 (m, 2H), 7.36 (d, J = 9.2 Hz, 1H), 4.83-4.82 (m, 1H), 4.66-4.61 (m, 1H), 4.16-4.13 (m, 4H), 3.87-3.86 (m, 1H), 3.61-3.59 (m, 1H), 3.37-3.34 (m, 4H), 3.15-3.12 (m, 1H), 2.94 (s, 3H), 2.36-2.30 (m, 1H), 2.15-1.98 (m, 3H). ¹⁹F NMR (376 MHz, methanol-d₄, ppm): δ −148.94 (1F). |
| 55 | 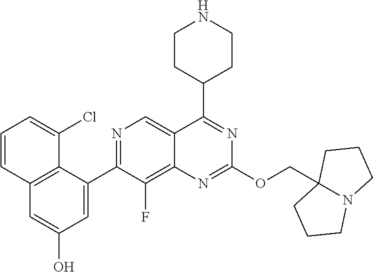 [SMILES: Oc1cc(-c2ncc3c(C4CCNCC4)nc(OCC45CCCN4CCC5)nc3c2F)c2c(Cl)cccc2c1] | 548.3 | 3 TFA salt, ¹H NMR (400 MHz, methanol-d₄, ppm): δ 9.51 (s, 1H), 7.78-7.76 (m, 1H), 7.40-7.31 (m, 3H), 7.15 (d, J = 2.4 Hz, 1H), 4.77 (s, 2H), 4.28-4.20 (m, 1H), 3.71-3.58 (m, 4H), 3.35-3.31 (m, 4H), 2.40-2.10 (m, 12H). |
| 57 | 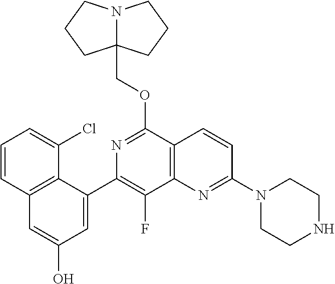 [SMILES: Oc1cc(-c2nc(OCC34CCCN3CCC4)c3ccc(N4CCNCC4)nc3c2F)c2c(Cl)cccc2c1] | 548.3 | 3 TFA salt, ¹H NMR (400 MHz, DMSO-d₆, ppm): δ 10.25 (s, 1H), 10.08 (brs, 1H), 8.89-8.72 (m, 2H), 8.30 (d, J = 9.6 Hz, 1H), 7.80 (d, J = 8.4 Hz 1H), 7.39-7.30 (m, 4H), 7.09 (d, J = 2.4 Hz, 1H), 4.48-4.36 (m, 2H), 4.10-3.90 (m, 4H), 3.53- 3.23 (m, 8H), 2.19-1.93 (m, 8H). ¹⁹F NMR (376 MHz, DMSO-d₆, ppm): δ −148.62 (1F). |
| 58 | 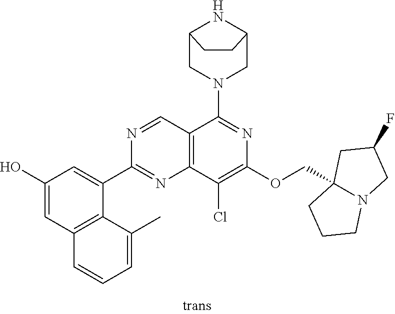 [SMILES: Cc1cccc2cc(O)cc(-c3ncc4c(N5CC6CCC(C5)N6)nc(OC[C@@]56CCCN5C[C@H](F)C6)c(Cl)c4n3)c12] | 589.3 | 3 TFA salt, ¹H NMR (400 MHz, methanol-d₄, ppm): δ 9.47 (s, 1H), 7.62 (d, J = 8.0 Hz, 1H), 7.33-7.28 (m, 2H), 7.15 (d, J = 2.8 Hz, 1H), 7.09 (d, J = 6.8 Hz, 1H), 5.71-5.58 (m, 1H), 4.77-4.65 (m, 2H), 4.54-4.52 (m, 2H), 4.25-4.20 (m, 2H), 3.98-3.84 (m, 5H), 3.53-3.50 (m, 1H), 2.77-2.21 (m, 10H), 1.95 (s, 3H). |
| 62 | 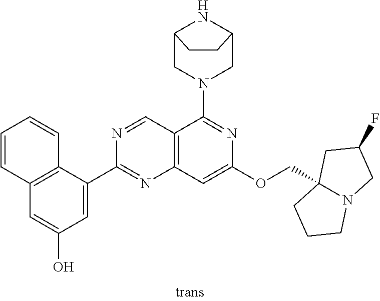 [SMILES: Oc1cc(-c2ncc3c(N4CC5CCC(C4)N5)nc(OC[C@@]45CCCN4C[C@H](F)C5)cc3n2)c2ccccc2c1] | 541.3 | 3 TFA salt, ¹H NMR (400 MHz, methanol-d₄, ppm): δ 9.53 (s, 1H), 8.36 (d, J = 8.4 Hz, 1H), 7.75 (d, J = 8.4 Hz, 1H), 7.65 (d, J = 2.8 Hz, 1H), 7.43 (t, J = 8.0 Hz, 1H), 7.31-7.29 (m, 2H), 6.77 (s, 1H), 5.65-5.53 (m, 1H), 4.70-4.58 (m, 4H), 4.22 (s, 2H), 3.94-3.82 (m, 5H), 3.53-3.48 (m, 1H), 2.65- 2.15 (m, 10H). |
| 63 | 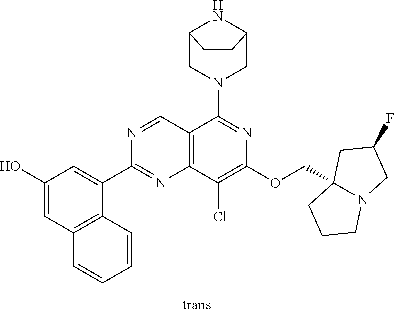 [SMILES: Oc1cc(-c2ncc3c(N4CC5CCC(C4)N5)nc(OC[C@@]45CCCN4C[C@H](F)C5)c(Cl)c3n2)c2ccccc2c1] | 575.3 | 3 TFA salt, ¹H NMR (400 MHz, methanol-d₄, ppm): δ 9.55 (s, 1H), 8.93 (d, J = 8.8 Hz, 1H), 7.96 (d, J = 2.8 Hz, 1H), 7.75 (d, J = 8.0 Hz, 1H), 7.44 (t, J = 7.2 Hz, 1H), 7.35-7.30 (m, 2H), 5.72-5.59 (m, 1H), 4.77-4.66 (m, 2H), 4.55-4.50 (m, 2H), 4.26-4.22 (m, 2H), 3.98-3.84 (m, 5H), 3.52-3.50 (m, 1H), 2.79-2.63 (m, 3H), 2.48-2.15 (m, 7H). |
| 64 | 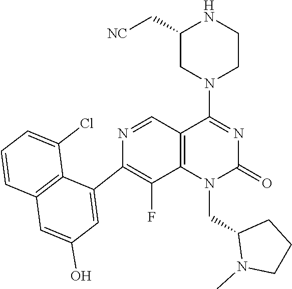 [SMILES: CN1CCC[C@H]1Cn1c(=O)nc(N2CCN[C@@H](CC#N)C2)c2cnc(-c3cc(O)cc4cccc(Cl)c34)c(F)c21] | 562.3 | 2 TFA salt, ¹H NMR (400 MHz, methanol-d₄, ppm): δ 8.93 (s, 1H), 7.76 (dd, J = 7.2, 2.0 Hz, 1H), 7.40-7.35 (m, 3H), 7.19-7.17 (m, 1H), 4.76-4.47 (m, 4H), 3.97-3.40 (m, 7H), 3.27-3.16 (m, 1H), 3.14-3.02 (m, 2H), 3.00-2.93 (m, 3H), 2.40-1.83 (m, 4H). ¹⁹F NMR (376 MHz, methanol-d₄, ppm): δ −134.90 (1F). |
| 65 | 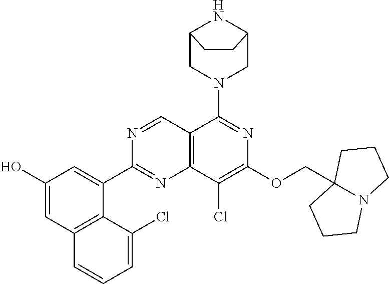 [SMILES: Oc1cc(-c2ncc3c(N4CC5CCC(C4)N5)nc(OCC45CCCN4CCC5)c(Cl)c3n2)c2c(Cl)cccc2c1] | 591.3 | 3 TFA salt, ¹H NMR (400 MHz, methanol-d₄, ppm): δ 9.42 (s, 1H), 7.74 (d, J = 7.2 Hz, 1H), 7.39-7.27 (m, 4H), 4.69 (d, J = 4.4 Hz, 2H), 4.53 (d, J = 13.6 Hz, 1H), 4.37 (d, J = 14.4 Hz, 1H), 4.23 (s, 2H), 3.92-3.82 (m, 2H), 3.75-3.69 (m, 2H), 3.35-3.32 (m, 2H), 2.41-2.35 (m, 2H), 2.30-2.11 (m, 10H). |
| 68 | 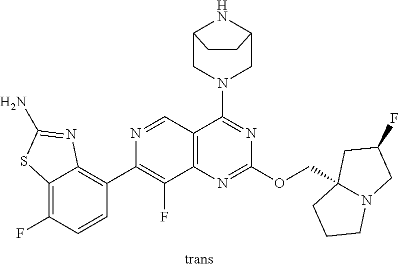 [SMILES: Nc1nc2c(-c3ncc4c(N5CC6CCC(C5)N6)nc(OC[C@@]56CCCN5C[C@H](F)C6)nc4c3F)ccc(F)c2s1] | 583.3 | 3 TFA salt, ¹H NMR (400 MHz, methanol-d₄, ppm): δ 9.17 (s, 1H), 7.71 (dd, J = 8.4, 5.6 Hz, 1H), 7.12 (t, J = 8.8 Hz, 1H), 5.65-5.47 (m, 1H), 4.94-4.87 (m, 2H), 4.74-4.65 (m, 2H), 4.30-4.18 (m, 2H), 4.06-3.83 (m, 5H), 3.50-3.43 (m, 1H), 2.80-2.53 (m, 2H), 2.46-2.30 (m, 3H), 2.23-2.02 (m, 5H). ¹⁹F NMR (376 MHz, methanol-d₄, ppm): δ −113.56 (1F), −138.30 (1F), −174.22 (1F). |
| 70 | 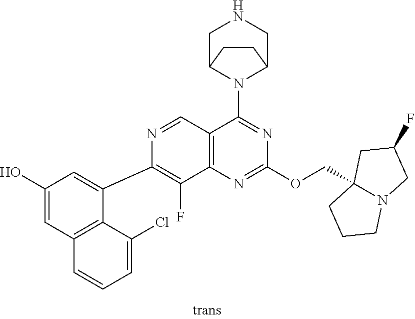 [SMILES: Oc1cc(-c2ncc3c(N4C5CCC4CNC5)nc(OC[C@@]45CCCN4C[C@H](F)C5)nc3c2F)c2c(Cl)cccc2c1] | 593.2 | 3 TFA salt, ¹H NMR (400 MHz, methanol-d₄, ppm): δ 9.16 (s, 1H), 7.76 (dd, J = 8.0, 1.2 Hz, 1H), 7.39-7.31 (m, 3H), 7.15-7.13 (m, 1H), 5.63-5.49 (m, 1H), 5.41-5.32 (m, 2H), 4.73-4.66 (m, 2H), 4.04-3.82 (m, 3H), 3.69-3.66 (m, 2H), 3.47-3.42 (m, 3H), 2.75-2.53 (m, 2H), 2.44-2.29 (m, 5H), 2.24-2.14 (m, 3H). ¹⁹F NMR (376 MHz, methanol-d₄, ppm): δ −139.41 (1F), −174.25 (1F). |
| 72 | 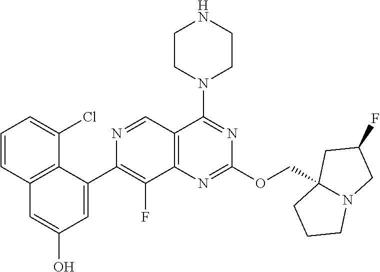 [SMILES: Oc1cc(-c2ncc3c(N4CCNCC4)nc(OC[C@@]45CCCN4C[C@H](F)C5)nc3c2F)c2c(Cl)cccc2c1] | 567.2 | 4 TFA salt, ¹H NMR (400 MHz, methanol-d₄, ppm): δ 9.12 (s, 1H), 7.75 (dd, J = 8.0, 1.2 Hz, 1H), 7.38-7.31 (m, 3H), 7.15-7.13 (m, 1H), 5.63-5.49 (m, 1H), 4.78-4.65 (m, 2H), 4.30-4.23 (m, 4H), 4.05-3.82 (m, 3H), 3.51-3.42 (m, 5H), 2.75-2.53 (m, 2H), 2.44-2.29 (m, 3H), 2.22-2.16 (m, 1H). ¹⁹F NMR (376 MHz, methanol-d₄, ppm): δ −139.42 (1F), −174.22 (1F). |
| 74 | 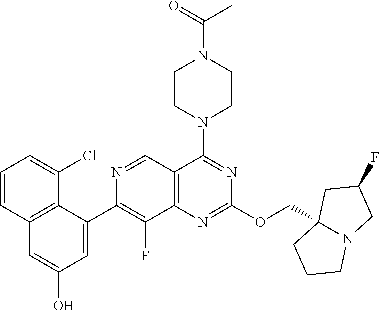 [SMILES: CC(=O)N1CCN(c2nc(OC[C@@]34CCCN3C[C@H](F)C4)nc3c(F)c(-c4cc(O)cc5cccc(Cl)c45)ncc23)CC1] | 609.3 | 2 TFA salt, ¹H NMR (400 MHz, methanol-d₄, ppm): δ 9.16 (s, 1H), 7.75 (dd, J = 8.0, 1.2 Hz, 1H), 7.38-7.31 (m, 3H), 7.15-7.13 (m, 1H), 5.63-5.49 (m, 1H), 4.75-4.62 (m, 2H), 4.26-4.18 (m, 4H), 4.05-3.83 (m, 7H), 3.45-3.42 (m, 1H), 2.74-2.53 (m, 2H), 2.44-2.29 (m, 3H), 2.19-2.14 (m, 4H). ¹⁹F NMR (376 MHz, methanol-d₄, ppm): δ −139.98 (1F), −174.23 (1F). |
| 77 | 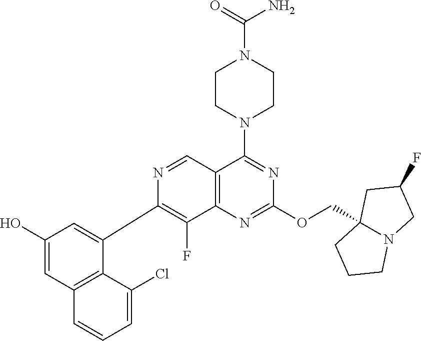 [SMILES: NC(=O)N1CCN(c2nc(OC[C@@]34CCCN3C[C@H](F)C4)nc3c(F)c(-c4cc(O)cc5cccc(Cl)c45)ncc23)CC1] | 610.3 | FA salt, ¹H NMR (400 MHz, methanol-d₄, ppm): δ 9.12 (s, 1H), 8.42 (brs, 1H), 7.74 (dd, J = 8.0, 1.2 Hz, 1H), 7.37-7.31 (m, 3H), 7.15-7.13 (m, 1H), 5.53-5.40 (m, 1H), 4.60-4.49 (m, 5H), 4.19-4.17 (m, 4H), 3.81-3.59 (m, 5H), 2.62-2.40 (m, 2H), 2.34-2.15 (m, 3H), 2.10-2.00 (m, 1H). |
| 78 | 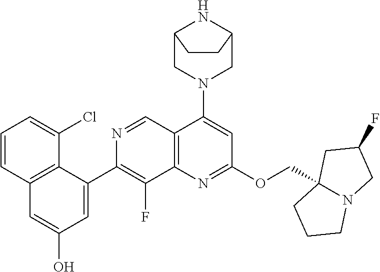 [SMILES: Oc1cc(-c2ncc3c(N4CC5CCC(C4)N5)cc(OC[C@@]45CCCN4C[C@H](F)C5)nc3c2F)c2c(Cl)cccc2c1] | 592.3 | 1.6 FA salt, ¹H NMR (400 MHz, methanol-d₄, ppm): δ 9.09 (s, 1H), 7.75 (d, J = 8.0 Hz, 1H), 7.37-7.29 (m, 3H), 7.14 (s, 1H), 6.73 (s, 1H), 5.48-5.34 (m, 1H), 4.60-4.49 (m, 2H), 4.17 (s, 2H), 3.74-3.68 (m, 2H), 3.62-3.52 (m, 3H), 3.43- 3.37 (m, 2H), 3.28-3.22 (m, 1H), 2.48-2.38 (m, 4H), 2.28- 2.12 (m, 5H), 2.05-1.95 (m, 1H). ¹⁹F NMR (376 MHz, methanol-d₄, ppm): δ −138.24 (1F), −174.16 (1F). |
| 79 | 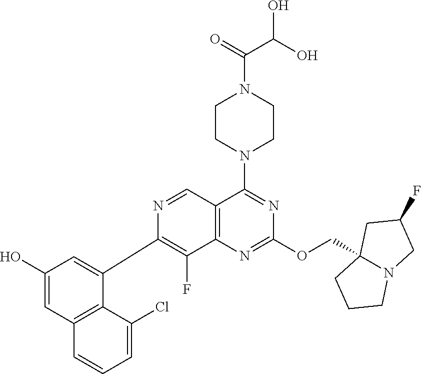 [SMILES: O=C(C(O)O)N1CCN(c2nc(OC[C@@]34CCCN3C[C@H](F)C4)nc3c(F)c(-c4cc(O)cc5cccc(Cl)c45)ncc23)CC1] | 641.2 | 2 FA salt, ¹H NMR (400 MHz, DMSO-d₆, ppm): δ 9.10 (s, 1H), 8.30 (brs, 2H), 7.80 (d, J = 8.0 Hz, 1H), 7.39-7.31 (m, 3H), 7.13-7.07 (m, 1H), 6.81-5.92 (m, 2H), 5.40-5.13 (m, 2H), 4.16-3.96 (m, 6H), 3.85-3.70 (m, 3H), 3.10-2.95 (m, 4H), 2.82-2.74 (m, 1H), 2.12-1.88 (m, 3H), 1.87-1.66 (m, 3H). |
| 80 | 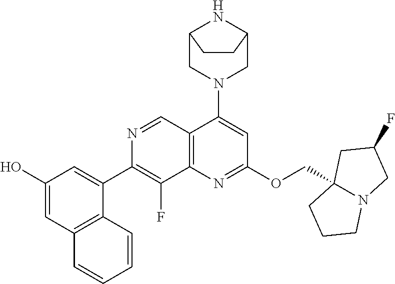 [SMILES: Oc1cc(-c2ncc3c(N4CC5CCC(C4)N5)cc(OC[C@@]45CCCN4C[C@H](F)C5)nc3c2F)c2ccccc2c1] | 558.3 | FA salt, ¹H NMR (400 MHz, methanol-d₄, ppm): δ 9.17 (s, 1H), 7.74 (d, J = 8.4 Hz, 1H), 7.47-7.39 (m, 3H), 7.28-7.19 (m, 3H), 6.69 (s, 1H), 5.41-5.27 (m, 1H), 4.49-4.39 (m, 2H), 4.05 (s, 2H), 3.69-3.66 (m, 2H), 3.42-3.30 (m, 5H), 3.15- 3.10 (m, 1H), 2.39-2.27 (m, 4H), 2.21-2.14 (m, 3H), 2.10- 1.93 (m, 3H). ¹⁹F NMR (376 MHz, methanol-d₄, ppm): δ −138.30 (1F), −173.97 (1F). |
| 82 | 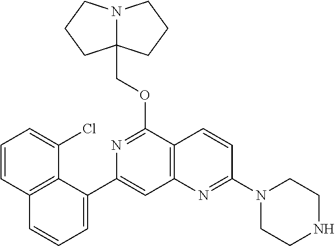 [SMILES: Clc1cccc2cccc(-c3cc4nc(N5CCNCC5)ccc4c(OCC45CCCN4CCC5)n3)c12] | 514.3 | ¹H NMR (400 MHz, methanol-d₄, ppm): δ 8.43-8.39 (m, 1H), 8.14-8.05 (m, 1H), 8.01-7.96 (m, 1H), 7.69-7.57 (m, 3H), 7.54-7.46 (m, 2H), 7.34-7.30 (m, 1H), 4.73-4.60 (m, 2H), 4.19-4.07 (m, 4H), 3.41-3.33 (m, 5H), 3.24-3.18 (m, 1H), 3.15-3.06 (m, 1H), 3.04-2.94 (m, 1H), 2.32-2.18 (m, 2H), 2.17-1.93 (m, 6H). |
| 83 | 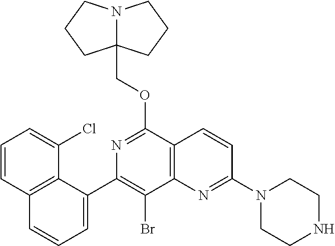 [SMILES: Clc1cccc2cccc(-c3nc(OCC45CCCN4CCC5)c4ccc(N5CCNCC5)nc4c3Br)c12] | 592.2 | ¹H NMR (400 MHz, methanol-d₄, ppm): δ 8.43-8.40 (m, 1H), 8.15-8.03 (m, 1H), 8.02-7.96 (m, 1H), 7.67-7.61 (m, 1H), 7.58-7.43 (m, 3H), 7.37-7.33 (m, 1H), 4.63-4.55 (m, 2H), 4.23-4.11 (m, 4H), 3.46-3.33 (m, 6H), 3.15-3.10 (m, 2H), 2.28-2.00 (m, 8H). |
| 85 | 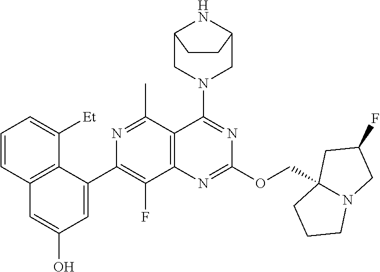 [SMILES: CCc1cccc2cc(O)cc(-c3nc(C)c4c(N5CC6CCC(C5)N6)nc(OC[C@@]56CCCN5C[C@H](F)C6)nc4c3F)c12] | 601.3 | 3 TFA salt, ¹H NMR (400 MHz, methanol-d₄, ppm): δ 7.61 (d, J = 8.4 Hz, 1H), 7.35 (t, J = 7.6 Hz, 1H), 7.29-7.25 (m, 1H), 7.14 (d, J = 7.2 Hz, 1H), 7.03-6.97 (m, 1H), 5.63-5.50 (m, 1H), 4.83-4.35 (m, 4H), 4.25-3.80 (m, 7H), 3.50-3.40 (m, 1H), 2.80-1.70 (m, 15H), 0.87 (t, J = 7.2 Hz, 3H). ¹⁹F NMR (376 MHz, methanol-d₄, ppm): δ −144.62 (1F), −174.17 (1F). |
| 87 | 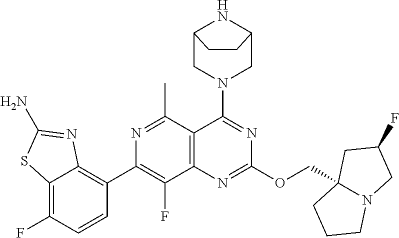 [SMILES: Cc1nc(-c2ccc(F)c3sc(N)nc23)c(F)c2nc(OC[C@@]34CCCN3C[C@H](F)C4)nc(N3CC4CCC(C3)N4)c12] | 597.3 | 4 TFA salt, ¹H NMR (400 MHz, methanol-d₄, ppm): δ 7.68- 7.64 (m, 1H), 7.08 (t, J = 8.8 Hz, 1H), 5.63-5.50 (m, 1H), 5.06-4.88 (m, 1H), 4.74-4.68 (m, 2H), 4.56-4.41 (m, 1H), 4.20-4.10 (m, 2H), 4.05-3.82 (m, 5H), 3.49-3.42 (m, 1H), 2.78-2.70 (m, 3H), 2.66-2.54 (m, 2H), 2.45-2.30 (m, 3H), 2.22-1.83 (m, 5H). ¹⁹F NMR (376 MHz, methanol-d₄, ppm): δ −113.25 (1F), −143.71 (1F), 174.16 (1F). |
| 88 | 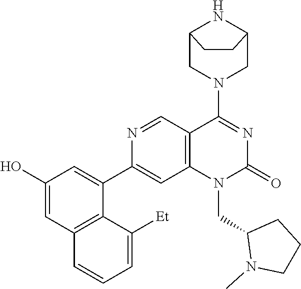 [SMILES: CCc1cccc2cc(O)cc(-c3cc4c(cn3)c(N3CC5CCC(C3)N5)nc(=O)n4C[C@@H]3CCCN3C)c12] | 525.4 | ¹H NMR (400 MHz, methanol-d₄, ppm): δ 9.02 (s, 1H), 7.75- 7.67 (m, 1H), 7.60 (d, J = 8.4 Hz, 1H), 7.36 (t, J = 7.6 Hz, 1H), 7.27-7.23 (m, 1H), 7.19 (d, J = 6.8 Hz, 1H), 7.01 (s, 1H), 4.80-4.50 (m, 4H), 4.28-4.13 (m, 2H), 4.00-3.66 (m, 4H), 3.25-3.12 (m, 1H), 3.04 (s, 3H), 2.41-1.72 (m, 10H), 0.93 (t, J = 6.8 Hz, 3H). |
| 89 | 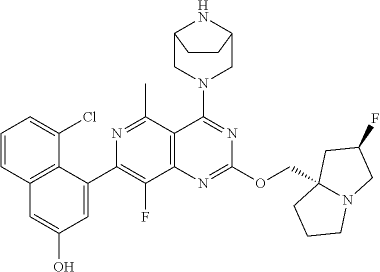 [SMILES: Cc1nc(-c2cc(O)cc3cccc(Cl)c23)c(F)c2nc(OC[C@@]34CCCN3C[C@H](F)C4)nc(N3CC4CCC(C3)N4)c12] | 607.3 | 3 TFA salt, ¹H NMR (400 MHz, methanol-d₄, ppm): δ 7.75 (d, J = 7.6 Hz, 1H), 7.40-7.27 (m, 3H), 7.16 (s, 1H), 5.67- 5.45 (m, 1H), 4.78-4.27 (m, 4H), 4.26-4.10 (m, 2H), 4.09- 3.80 (m, 5H), 3.52-3.40 (m, 1H), 2.81-2.50 (m, 5H), 2.48- 2.27 (m, 3H), 2.25-1.67 (m, 5H). ¹⁹F NMR (376 MHz, methanol-d₄, ppm): δ −145.1 (1F), −174.2 (1F). |
| 90 | 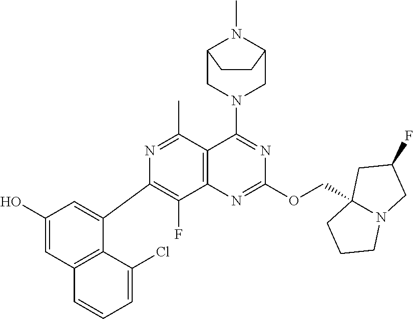 [SMILES: Cc1nc(-c2cc(O)cc3cccc(Cl)c23)c(F)c2nc(OC[C@@]34CCCN3C[C@H](F)C4)nc(N3CC4CCC(C3)N4C)c12] | 621.3 | 4TFA salt, ¹H NMR (400 MHz, methanol-d₄, ppm): δ 7.74 (d, J = 8.0 Hz, 1H), 7.40-7.27 (m, 3H), 7.16 (s, 1H), 5.67- 5.45 (m, 1H), 4.75-4.39 (m, 4H), 4.14-3.80 (m, 7H), 3.51- 3.39 (m, 1H), 2.88 (s, 3H), 2.78-2.54 (m, 5H), 2.46-2.14 (m, 6H), 1.97-1.74 (m, 2H). ¹⁹F NMR (376 MHz, methanol-d₄, ppm): δ −145.0 (1F), −174.2 (1F). |
| 93 | 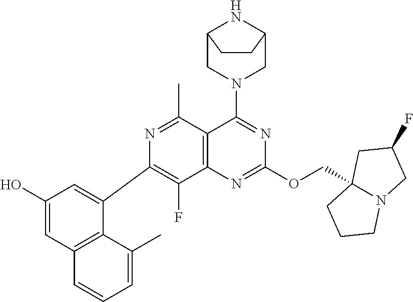 [SMILES: Cc1cccc2cc(O)cc(-c3nc(C)c4c(N5CC6CCC(C5)N6)nc(OC[C@@]56CCCN5C[C@H](F)C6)nc4c3F)c12] | 587.3 | 3 TFA salt, ¹H NMR (400 MHz, methanol-d₄, ppm): δ 7.62- 7.60 (m, 1H), 7.32-7.27 (m, 2H), 7.07-7.01 (m, 2H), 5.63- 5.50 (m, 1H), 5.08-4.95 (m, 1H), 4.73-4.66 (m, 2H), 4.56- 4.51 (m, 1H), 4.19-4.13 (m, 2H), 4.05-3.82 (m, 5H), 3.49- 3.42 (m, 1H), 2.77-2.53 (m, 5H), 2.45-2.30 (m, 3H), 2.22- 1.79 (m, 8H). ¹⁹F NMR (376 MHz, methanol-d₄, ppm): δ −144.95 (1F), −174.15(1F). |
| 94 | 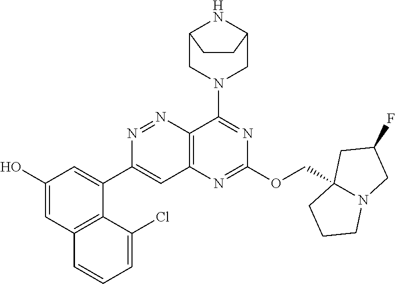 [SMILES: Oc1cc(-c2cc3nc(OC[C@@]45CCCN4C[C@H](F)C5)nc(N4CC5CCC(C4)N5)c3nn2)c2c(Cl)cccc2c1] | 576.3 | ¹H NMR (400 MHz, methanol-d₄, ppm): δ 7.79-7.76 (m, 2H), 7.41-7.32 (m, 3H), 7.18 (d, J = 2.4 Hz, 1 H), 6.81-6.11 (m, 1H), 5.64-5.03 (m, 2H), 4.75-4.63 (m, 2H), 4.35-4.30 (m, 2H), 4.09-3.58 (m, 5H), 3.52-3.41 (m, 1H), 2.76-2.54 (m, 2H), 2.46-2.30 (m, 3H), 2.27-2.03 (m, 5H). |
| 95 | 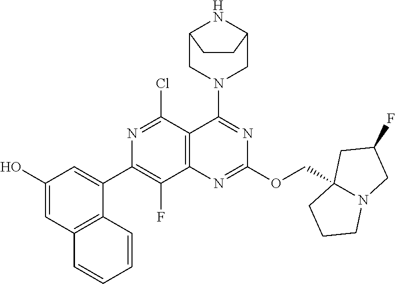 [SMILES: Oc1cc(-c2nc(Cl)c3c(N4CC5CCC(C4)N5)nc(OC[C@@]45CCCN4C[C@H](F)C5)nc3c2F)c2ccccc2c1] | 593.3 | 6 TFA salt, ¹H NMR (400 MHz, methanol-d₄, ppm): δ 7.75 (d, J = 8.4 Hz, 1H), 7.65 (d, J = 8.0 Hz,1H), 7.42 (t, J = 7.2 Hz, 1H), 7.32-7.23 (m, 3H), 5.64-5.50 (m, 1H), 4.74-4.51 (m, 4H), 4.17-4.11 (m, 2H), 4.10-3.99 (m, 2H), 3.95-3.83 (m, 3H), 3.49-3.43 (m, 1H), 2.77-2.54 (m, 2H), 2.51-2.30 (m, 3H), 2.28-1.78 (m, 5H). ¹⁹F NMR (376 MHz, methanol- d₄, ppm): δ −141.54 (1F), −174.20 (1F). |
| 97 | 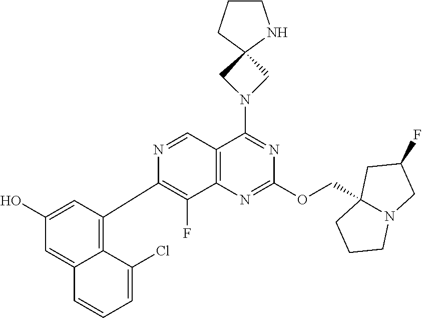 | 593.2 | 3 TFA salt, ¹H NMR (400 MHz, methanol-d₄, ppm): δ 8.92 (s, 1H), 7.75 (d, J = 8.0 Hz, 1H), 7.39-7.30 (m, 3H), 7.12 (d, J = 2.4 Hz, 1H), 5.62-5.48 (m, 1H), 5.22-4.95 (m, 2H), 4.78- 4.63 (m, 4H), 4.04-3.82 (m, 3H), 3.50-3.42 (m, 3H), 2.72- 2.52 (m, 2H), 2.45-2.29 (m, 5H), 2.20-2.12 (m, 3H). ¹⁹F NMR (376 MHz, methanol-d₄, ppm): δ −139.34 (1F), −174.26 (1F). |
| 99 | 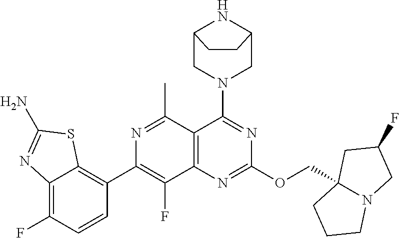 [SMILES: Cc1nc(-c2ccc(F)c3nc(N)sc23)c(F)c2nc(OC[C@@]34CCCN3C[C@H](F)C4)nc(N3CC4CCC(C3)N4)c12] | 597.3 | 4 TFA salt, ¹H NMR (400 MHz, methanol-d₄, ppm): δ 8.06 (dd, J = 8.8, 4.4 Hz, 1H), 7.34-7.30 (m, 1H), 5.68-5.48 (m, 1H), 4.75-4.36 (m, 4H), 4.20-4.10 (m, 2H), 4.09-3.82 (m, 5H), 3.52-3.42 (m, 1H), 2.89-2.50 (m, 5H), 2.48-2.29 (m, 3H), 2.26-1.68 (m, 5H). ¹⁹F NMR (376 MHz, methanol-d₄, ppm): δ −126.70 (1F), −145.80 (1F), −174.20 (1F). |
| 100 | 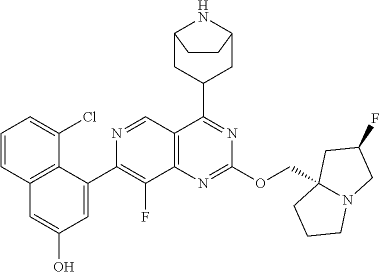 [SMILES: Oc1cc(-c2ncc3c(C4CC5CCC(C4)N5)nc(OC[C@@]45CCCN4C[C@H](F)C5)nc3c2F)c2c(Cl)cccc2c1] | 592.2 | 3 TFA salt, ¹H NMR (400 MHz, methanol-d₄, ppm): δ 9.52 (s, 1H), 7.77-7.75 (m, 1H), 7.39-7.30 (m, 3H), 7.15 (d, J = 2.8 Hz, 1H), 5.65-5.52 (m, 1H), 4.94-4.72 (m, 2H), 4.47-4.40 (m, 1H), 4.25-4.18 (m, 2H), 3.99-3.88 (m, 3H), 3.51-3.45 (m, 1H), 2.78-2.14 (m, 14H). ¹⁹F NMR (376 MHz, methanol-d₄, ppm): δ −139.29 (1F), −174.32 (1F). |
| 101 | 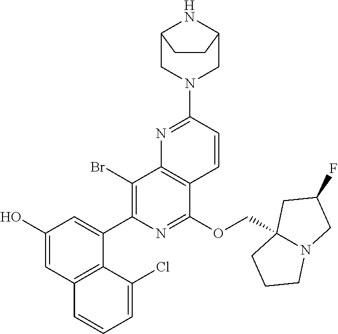 [SMILES: Oc1cc(-c2nc(OC[C@@]34CCCN3C[C@H](F)C4)c3ccc(N4CC5CCC(C4)N5)nc3c2Br)c2c(Cl)cccc2c1] | 652.2 | 4 TFA salt, ¹H NMR (400 MHz, methanol-d₄, ppm): δ 8.40 (d, J = 9.2 Hz, 1H), 7.74-7.71 (m, 1H), 7.36-7.27 (m, 4H), 7.12-7.04 (m, 1H), 5.58-5.38 (m, 1H), 4.73-4.62 (m, 4H), 4.26-4.22 (m, 2H), 3.75-3.42 (m, 5H), 3.12-3.10 (m, 1H), 2.68-2.52 (m, 2H), 2.37-2.21 (m, 2H), 2.19-2.02 (m, 6H). |
| 104 | 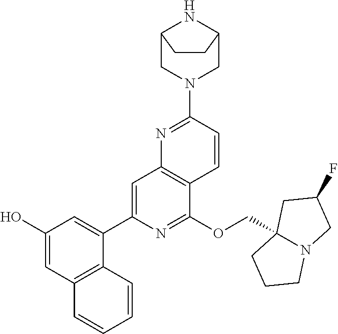 [SMILES: Oc1cc(-c2cc3nc(N4CC5CCC(C4)N5)ccc3c(OC[C@@]34CCCN3C[C@H](F)C4)n2)c2ccccc2c1] | 540.3 | 3 TFA salt, ¹H NMR (400 MHz, methanol-d₄, ppm): δ 8.40 (d, J = 9.2 Hz, 1H), 7.93 (d, J = 8.4 Hz, 1H), 7.74 (d, J = 8.3 Hz, 1H), 7.47-7.37 (m, 2H), 7.31-7.18 (m, 4H), 5.60-5.40 (m, 1H), 4.67-4.61 (m, 4H), 4.28-4.20 (m, 2H), 3.71-3.42 (m, 5H), 3.04-2.92 (m, 1H), 2.63-2.50 (m, 2H), 2.30-2.20 (m, 2H), 2.20-1.99 (m, 6H). |
| 105 | 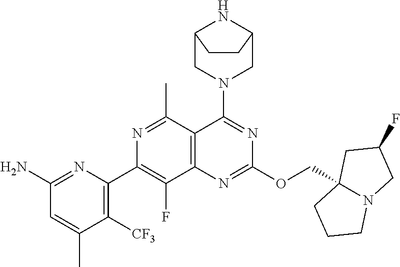 [SMILES: Cc1cc(N)nc(-c2nc(C)c3c(N4CC5CCC(C4)N5)nc(OC[C@@]45CCCN4C[C@H](F)C5)nc3c2F)c1C(F)(F)F] | 605.3 | ¹H NMR (400 MHz, methanol-d₄, ppm): δ 6.67 (s, 1H), 5.68- 5.47 (m, 1H), 4.68 (s, 2H), 4.60-4.29 (m, 1H), 4.15 (brs, 2H), 4.05-3.81 (m, 5H), 3.48-3.41 (m, 1H), 2.77-2.52 (m, 5H), 2.45-2.40 (m, 4H), 2.39-2.12 (m, 4H), 2.08-1.65 (m, 4H). ¹⁹F NMR (376 MHz, methanol-d₄, ppm): δ −56.13 (3F), −147.72 (1F), −173.12 (1F). |
| 110 | 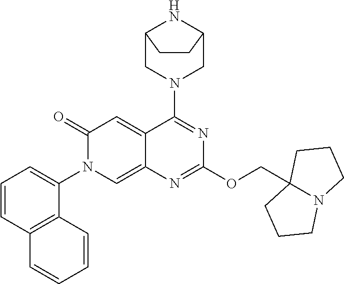 [SMILES: O=c1cc2c(N3CC4CCC(C3)N4)nc(OCC34CCCN3CCC4)nc2cn1-c1cccc2ccccc12] | 523.3 | ¹H NMR (400 MHz, methanol-d₄, ppm): δ 8.74 (s, 1H), 7.99 (d, J = 8.4 Hz, 1H), 7.95 (d, J = 8 Hz, 1H), 7.78 (d, J = 8.4 Hz, 1H), 7.54-7.47 (m, 3H), 7.31 (s, 1H), 7.18 (d, J = 7.6 Hz, 1H), 4.60 (s, 2H), 4.50 (d, J = 13.6 Hz, 2H), 4.13 (s, 2H), 3.68-3.64 (m, 4H), 3.29-3.26 (m, 2H), 2.33-1.95 (m, 12H). |
| 111 | 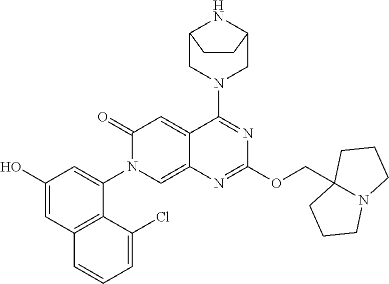 [SMILES: O=c1cc2c(N3CC4CCC(C3)N4)nc(OCC34CCCN3CCC4)nc2cn1-c1cc(O)cc2cccc(Cl)c12] | 573.3 | ¹H NMR (400 MHz, methanol-d₄, ppm): δ 8.70 (s, 1H), 7.69- 7.67 (m, 1H), 7.31-7.27 (m, 2H), 7.21 (s, 1H), 7.14 (d, J = 2.4 Hz, 1H), 6.88 (d, J = 2.4 Hz, 1H), 4.60 (s, 2H), 4.48 (d, J = 13.6 Hz, 2H), 4.16 (s, 2H), 3.71-3.64 (m, 4H), 3.29-3.26 (m, 2H), 2.32-1.99 (m, 12H). |
| 112 | 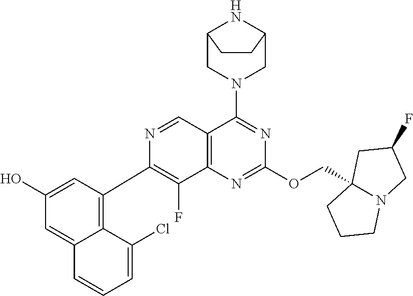 [SMILES: Oc1cc(-c2ncc3c(N4CC5CCC(C4)N5)nc(OC[C@@]45CCCN4C[C@H](F)C5)nc3c2F)c2c(Cl)cccc2c1] | 593.3 | 3 TFA salt, ¹H NMR (400 MHz, methanol-d₄, ppm): δ 9.10 (s, 1H), 7.76 (dd, J = 8.0, 1.2 Hz, 1H), 7.38-7.31 (m, 3H), 7.14-7.13 (m, 1H), 5.63-5.48 (m, 1H), 4.88-4.83 (m, 2H), 4.69-4.69 (m, 2H), 4.25 (brs, 2H), 4.04-3.83 (m, 5H), 3.49- 3.42 (m, 1H), 2.76-2.53 (m, 2H), 2.45-2.29 (m, 3H), 2.23- 2.07 (m, 5H). ¹⁹F NMR (376 MHz methanol-d₄, ppm): δ −139.31 (1F), −174.21 (1F). |
| 113 | 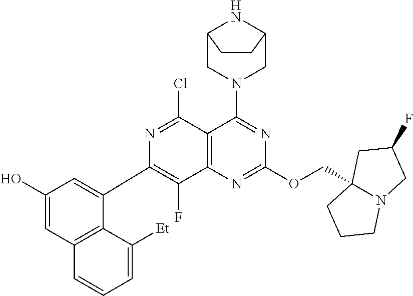 [SMILES: CCc1cccc2cc(O)cc(-c3nc(Cl)c4c(N5CC6CCC(C5)N6)nc(OC[C@@]56CCCN5C[C@H](F)C6)nc4c3F)c12] | 621.3 | 4 TFA salt, ¹H NMR (400 MHz, methanol-d₄, ppm): δ 7.62 (d, J = 8.2 Hz, 1H), 7.39-7.33 (m, 1H), 7.28 (d, J = 2.8 Hz, 1H), 7.17 (d, J = 7.2 Hz, 1H), 7.02 (d, J = 2 Hz, 1H), 5.69- 5.47 (m, 1H), 4.70 (s, 2H), 4.47 (br, 1H), 4.30-4.13 (m, 2H), 4.11-3.78 (m, 5H), 3.54-3.41 (m, 1H), 2.83-2.53 (m, 2H), 2.52-2.14 (m, 7H), 2.13-1.62 (m, 4H), 0.92 (t, J = 7.4 Hz, 3H). ¹⁹F NMR (376 MHz, methanol-d₄, ppm): δ −141.76 (1F), −174.21 (1F). |
| 114 | 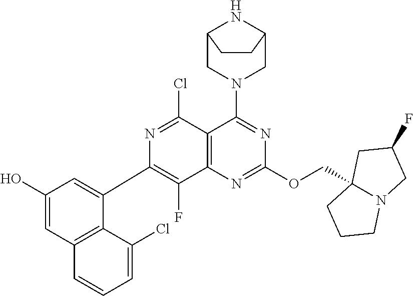 [SMILES: Oc1cc(-c2nc(Cl)c3c(N4CC5CCC(C4)N5)nc(OC[C@@]45CCCN4C[C@H](F)C5)nc3c2F)c2c(Cl)cccc2c1] | 627.3 | ¹H NMR (400 MHz, methanol-d₄, ppm): δ 7.62 (d, J = 1.6 Hz, 1H), 7.42-7.33 (m, 3H), 7.18 (t, J = 2.0 Hz, 1H), 5.64- 5.50 (m, 1H), 4.74-4.65 (m, 4H), 4.20-4.18 (m, 2H), 4.03- 3.83 (m, 5H), 3.50-3.43 (m, 1H), 2.76-2.57 (m, 3H), 2.45- 2.32 (m, 3H), 2.31-2.17 (m, 4H). |
| 115 | 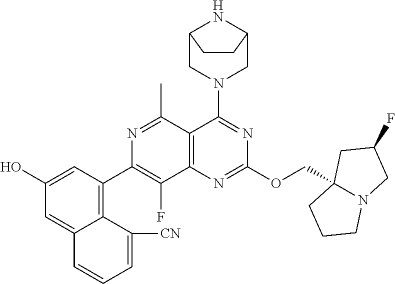 [SMILES: Cc1nc(-c2cc(O)cc3cccc(C#N)c23)c(F)c2nc(OC[C@@]34CCCN3C[C@H](F)C4)nc(N3CC4CCC(C3)N4)c12] | 598.3 | ¹H NMR (400 MHz, methanol-d₄, ppm): δ 8.09 (d, J = 8.4 Hz, 1H), 7.71 (d, J = 7.2 Hz, 1H), 7.52 (t, J = 7.6 Hz, 1H), 7.43-7.40 (m, 2H), 5.63-5.50 (m, 1H), 5.28-4.94 (m, 1H), 4.72-4.66 (m, 2H), 4.28-3.71 (m, 8H), 3.49-3.42 (m, 1H), 2.78-2.53 (m, 5H), 2.49-2.30 (m, 3H), 2.23-1.87 (m, 5H). |
| 117 | 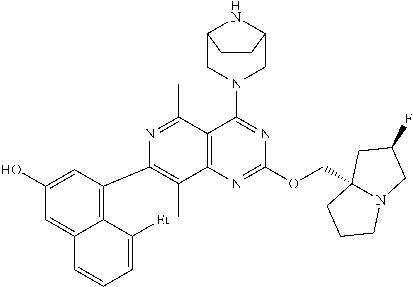 [SMILES: CCc1cccc2cc(O)cc(-c3nc(C)c4c(N5CC6CCC(C5)N6)nc(OC[C@@]56CCCN5C[C@H](F)C6)nc4c3C)c12] | 597.4 | ¹H NMR (400 MHz, methanol-d₄, ppm): δ 7.66 (d, J = 8.4 Hz, 1H), 7.39 (t, J = 7.6 Hz, 1H), 7.33 (d, J = 2.4 Hz, 1H), 7.20 (d, J = 7.2 Hz, 1H), 7.01 (d, J = 2.8 Hz, 1H), 5.66-5.52 (m, 1H), 4.79-4.71 (m, 2H), 4.59-4.30 (m, 2H), 4.26-4.11 (m, 2H), 4.08-3.83 (m, 5H) 3.50-3.43 (m, 1H), 2.81-2.55 (m, 5H), 2.47-2.31 (m, 3H), 2.27-2.18 (m, 6H), 2.03-1.85 (m, 4H), 0.92 (t, J = 7.2 Hz, 3H). ¹⁹F NMR (376 MHz, methanol-d₄, ppm): δ −174.27 (1F). |
| 118 | 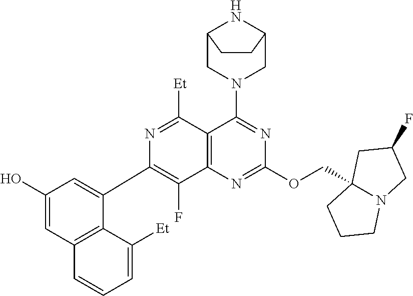 [SMILES: CCc1cccc2cc(O)cc(-c3nc(CC)c4c(N5CC6CCC(C5)N6)nc(OC[C@@]56CCCN5C[C@H](F)C6)nc4c3F)c12] | 615.3 | 4 TFA salt, ¹H NMR (400 MHz, methanol-d₄, ppm): δ 7.62 (d, J = 8.0 Hz, 1H), 7.35 (t, J = 8.0 Hz, 1H), 7.27 (d, J = 2.4 Hz, 1H), 7.14 (d, J =7.2 Hz, 1H), 7.00 (s, 1H), 5.63-5.50 (m, 1H), 4.72-4.66 (m, 2H), 4.20-3.86 (m, 7H), 3.49-3.42 (m, 1H), 3.35-3.25 (m, 1H), 3.04-3.01 (m, 2H), 2.73-2.53 (m, 2H), 2.42-1.76 (m, 10H), 1.35-1.26 (m, 1H), 1.17 (t, J = 7.2 Hz, 3H), 0.86 (t, J = 7.2 Hz, 3H). ¹⁹F NMR (376 MHz, methanol-d₄, ppm): δ −147.80 (1F), −174.17 (1F). |
| 120 | 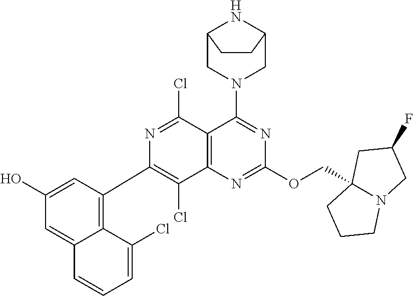 [SMILES: Oc1cc(-c2nc(Cl)c3c(N4CC5CCC(C4)N5)nc(OC[C@@]45CCCN4C[C@H](F)C5)nc3c2Cl)c2c(Cl)cccc2c1] | 643.2 | ¹H NMR (400 MHz, methanol-d₄, ppm): δ 7.74-7.71 (m, 1H), 7.39-7.29 (m, 3H), 7.12 (s, 1H), 5.52-5.29 (m, 1H), 4.60-4.41 (m, 3H), 4.10-3.90 (m, 2H), 3.85-3.70 (m, 2H), 3.65-3.41 (m, 4H), 3.23-3.18 (m, 1H), 2.54-2.20 (m, 4H), 2.20-1.94 (m, 4H), 1.94-1.75 (m, 2H). |
| 121 | 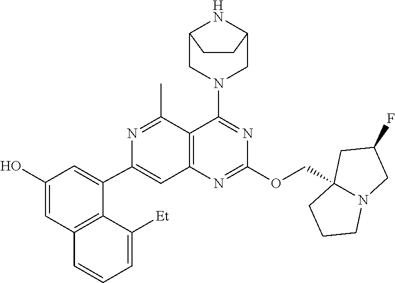 [SMILES: CCc1cccc2cc(O)cc(-c3cc4nc(OC[C@@]56CCCN5C[C@H](F)C6)nc(N5CC6CCC(C5)N6)c4c(C)n3)c12] | 583.4 | ¹H NMR (400 MHz, methanol-d₄, ppm): δ 7.62 (d, J = 7.6 Hz, 1H), 7.49 (s, 1H), 7.37 (t, J = 7.6 Hz, 1H), 7.28 (d, J = 2.8 Hz, 1H), 7.19 (d, J = 7.2 Hz, 1H), 7.05 (d, J = 2.4 Hz, 1H), 5.65-5.52 (m, 1H), 4.74-4.67 (m, 2H), 4.62-4.29 (m, 2H), 4.22-4.11 (m, 2H), 4.08-3.87 (m, 5H), 3.50-3.43 (m, 1H), 2.81-2.71 (m, 3H), 2.67-2.51 (m, 2H), 2.43-2.31 (m, 4H), 2.29-2.14 (m, 2H), 2.13-1.98 (m, 2H), 1.94-1.74 (m, 2H), 0.90 (t, J = 7.2 Hz, 3H). |
| 126 | 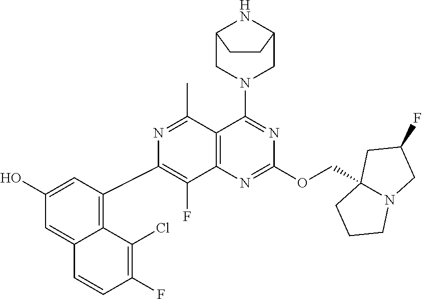 [SMILES: Cc1nc(-c2cc(O)cc3ccc(F)c(Cl)c23)c(F)c2nc(OC[C@@]34CCCN3C[C@H](F)C4)nc(N3CC4CCC(C3)N4)c12] | 625.3 | ¹H NMR (400 MHz, methanol-d₄, ppm): δ 7.82 (dd, J = 9.2, 5.6 Hz, 1H), 7.43-7.38 (m, 2H), 7.27-7.23 (m, 1H), 5.66-5.52 (m, 1H), 4.72-4.70 (m, 2H), 4.65-4.52 (m, 1H), 4.27-3.84 (m, 8H), 3.50-3.43 (m, 1H), 2.79-2.55 (m, 5H), 2.47-2.31 (m, 3H), 2.28-1.76 (m, 5H). ¹⁹F NMR (376 MHz, methanol- d₄, ppm): δ −116.52 (1F), −144.28 (1F), −174.20 (1F). |
| 129 | 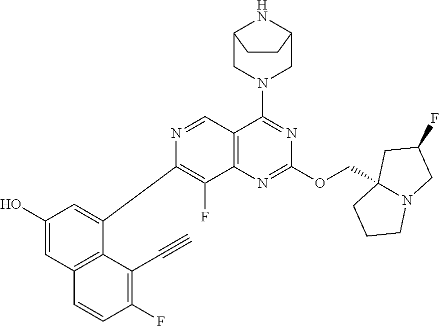 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] | 601.3 | 3 TFA salt, ¹H NMR (400 MHz, methanol-d₄, ppm): δ 9.11 (s, 1H), 7.93-7.84 (m, 1H), 7.41-7.29 (m, 2H), 7.21 (s, 1H), 5.69-5.49 (m, 1H), 4.87-4.84 (m, 2H), 4.78-4.65 (m, 2H), 4.35-4.24 (m, 2H), 4.09-3.83 (m, 5H), 3.53-3.43 (m, 1H), 3.38-3.32 (m, 1H), 2.79-2.53 (m, 2H), 2.50-2.28 (m, 3H), 2.24-2.05 (m, 5H). ¹⁹F NMR (376 MHz, methanol-d₄, ppm): δ −111.6(1F), −139.2 (1F), −174.2 (1F). |
| 130 | 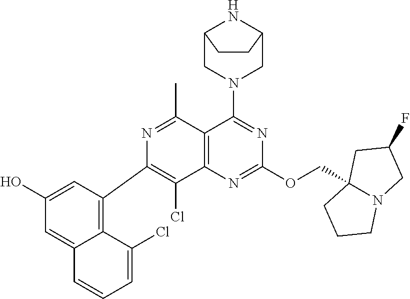 [SMILES: Cc1nc(-c2cc(O)cc3cccc(Cl)c23)c(Cl)c2nc(OC[C@@]34CCCN3C[C@H](F)C4)nc(N3CC4CCC(C3)N4)c12] | 623.2 | ¹H NMR (400 MHz, methanol-d₄, ppm):7.73 (d, J = 7.2 Hz, 1H), 7.36-7.28 (m, 3H), 7.12 (s, 1H), 5.52-5.38 (m, 1H), 4.59-4.48 (m, 2H), 3.94-3.72 (m, 5H), 3.68-3.56 (m, 3H), 3.31-3.23 (m, 1H), 2.65-2.54 (m, 4H), 2.48-2.25 (m, 3H), 2.23-2.15 (m, 2H), 2.10-1.95 (m, 1H), 1.93-1.70 (m, 3H), 1.65-1.40 (m, 1H). |
| 131 | 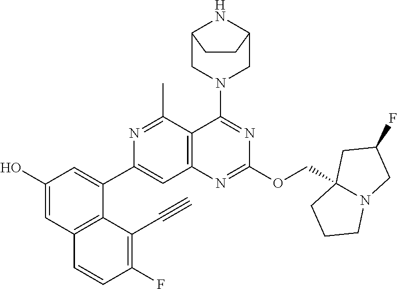 [SMILES: C#Cc1c(F)ccc2cc(O)cc(-c3cc4nc(OC[C@@]56CCCN5C[C@H](F)C6)nc(N5CC6CCC(C5)N6)c4c(C)n3)c12] | 597.3 | 3 TFA salt, ¹H NMR (400 MHz, methanol-d₄, ppm): δ 7.93- 7.89 (m, 1H), 7.59 (s, 1H), 7.41-7.36 (m, 2H), 7.29-7.28 (m, 1H), 5.66-5.53 (m, 1H), 4.79-4.68 (m, 2H), 4.58-4.40 (m, 1H), 4.35-3.75 (m, 8H), 3.53-3.44 (m, 2H), 2.81-2.57 (m, 5H), 2.47-2.32 (m, 3H), 2.28-1.72 (m, 5H). ¹⁹F NMR (376 MHz, methanol-d₄, ppm): δ −110.69 (1F), −174.17 (1F). |
| 132 | 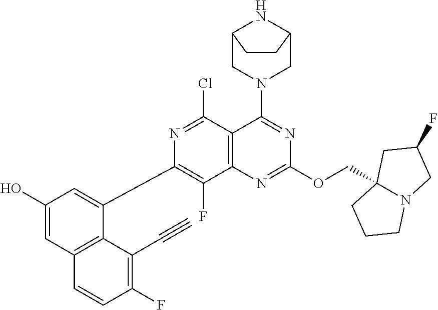 [SMILES: C#Cc1c(F)ccc2cc(O)cc(-c3nc(Cl)c4c(N5CC6CCC(C5)N6)nc(OC[C@@]56CCCN5C[C@H](F)C6)nc4c3F)c12] | 635.3 | 3 TFA salt, ¹H NMR (400 MHz, methanol-d₄, ppm): δ 7.90- 7.86 (m, 1H), 7.37-7.32 (m, 2H), 7.23 (s, 1H), 5.65-5.52 (m, 1H), 4.74-4.71 (m, 2H), 4.64-4.41 (m, 1H), 4.24-3.84 (m, 8H), 3.50-3.40 (m, 2H), 2.78-2.55 (m, 2H), 2.47-2.31 (m, 3H), 2.24-1.71 (m, 5H). ¹⁹F NMR (376 MHz, methanol-d₄, ppm): δ −111.39 (1F), −141.57 (1F), −174.20 (1F). |
| 133 | 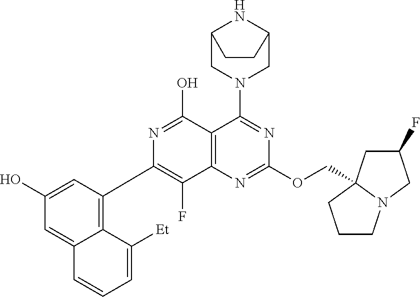 [SMILES: CCc1cccc2cc(O)cc(-c3nc(O)c4c(N5CC6CCC(C5)N6)nc(OC[C@@]56CCCN5C[C@H](F)C6)nc4c3F)c12] | 603.3 | 3TFA salt, ¹H NMR (400 MHz, methanol-d₄, ppm): δ 7.64 (d, J = 8.0 Hz, 1H), 7.40 (t, J = 7.6 Hz, 1H), 7.33 (d, J = 2.4 Hz, 1H), 7.24 (d, J = 7.2 Hz, 1H), 7.12 (d, J = 2.4 Hz, 1H), 5.64-5.51 (m, 1H), 4.69-4.39 (m, 4H), 4.23-4.14 (m, 2H), 4.09-3.83 (m, 3H), 3.67-3.58 (m, 2H), 3.51-3.41 (m, 1H), 2.78-2.54 (m, 4H), 2.42-2.09 (m, 8H), 1.08 (t, J = 7.2 Hz, 3H). ¹⁹F NMR (376 MHz, methanol-d₄, ppm): δ −161.72 (1F), −174.14 (1F). |
| 134 | 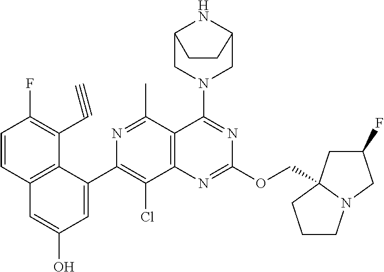 [SMILES: C#Cc1c(F)ccc2cc(O)cc(-c3nc(C)c4c(N5CC6CCC(C5)N6)nc(OC[C@@]56CCCN5C[C@H](F)C6)nc4c3Cl)c12] | 631.3 | 3 TFA salt, ¹H NMR (400 MHz, methanol-d₄, ppm): δ 7.89- 7.85 (m, 1H), 7.35-7.30 (m, 2H), 7.14 (s, 1H), 5.63-5.50 (m, 1H), 4.82-4.69 (m, 2H), 4.67-4.27 (m, 2H), 4.25-4.13 (m, 2H), 4.09-3.83 (m, 5H), 3.51-3.44 (m, 1H), 3.24-3.19 (m, 1H), 2.79-2.53 (m, 5H), 2.51-2.29 (m, 3H), 2.27-1.68 (m, 5H). ¹⁹F NMR (376 MHz, methanol-d₄, ppm): δ −111.68 (1F), −174.20 (1F). |
| 135 | 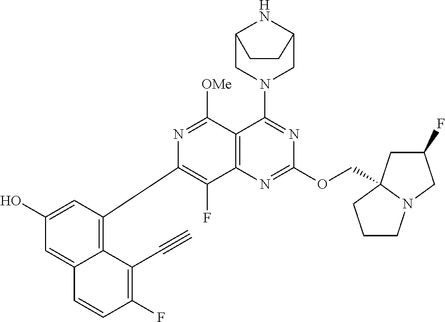 [SMILES: C#Cc1c(F)ccc2cc(O)cc(-c3nc(OC)c4c(N5CC6CCC(C5)N6)nc(OC[C@@]56CCCN5C[C@H](F)C6)nc4c3F)c12] | 631.3 | 3 TFA salt, ¹H NMR (400 MHz, methanol-d₄, ppm): δ 7.87- 7.82 (m, 1H), 7.33-7.28 (m, 2H), 7.22 (s, 1H), 5.67-5.46 (m, 1H), 4.73-4.63 (m, 2H), 4.53-4.38 (m, 2H), 4.27-4.18 (m, 2H), 4.11-3.72 (m, 8H), 3.49-3.34 (m, 2H), 2.69-2.51 (m, 2H), 2.48-2.27 (m, 3H), 2.25-2.02 (m, 5H). ¹⁹F NMR (376 MHz, methanol-d₄, ppm): δ −111.78 (1F), −150.61 (1F), −174.28 (1F). |
| 137 | 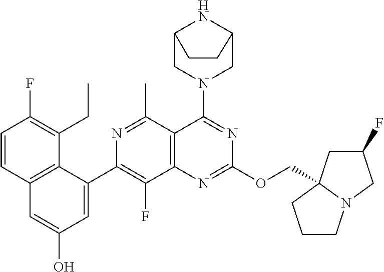 [SMILES: CCc1c(F)ccc2cc(O)cc(-c3nc(C)c4c(N5CC6CCC(C5)N6)nc(OC[C@@]56CCCN5C[C@H](F)C6)nc4c3F)c12] | 619.3 | 3 TFA salt, ¹H NMR (400 MHz, methanol-d₄, ppm): δ 7.68 (dd, J = 8.8, 6.0 Hz, 1H), 7.31 (d, J = 2.8 Hz, 1H), 7.25 (t, J = 9.6 Hz, 1H), 7.06 (s, 1H), 5.65-5.52 (m, 1H), 4.79-4.31 (m, 4H), 4.25-3.84 (m, 7H), 3.50-3.41 (m, 1H), 2.81-2.55 (m, 5H), 2.53-2.29 (m, 4H), 2.25-1.69 (6H), 1.35-1.25 (m, 3H). ¹⁹F NMR (376 MHz, methanol-d₄, ppm): δ 121.04 (1F), −144.31 (1F), −174.14 (1F). |
| 138 | 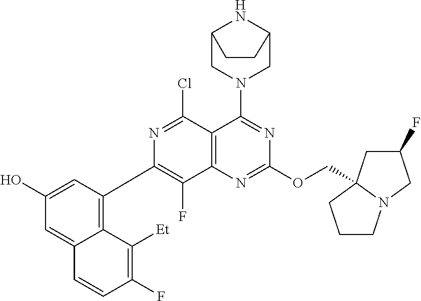 [SMILES: CCc1c(F)ccc2cc(O)cc(-c3nc(Cl)c4c(N5CC6CCC(C5)N6)nc(OC[C@@]56CCCN5C[C@H](F)C6)nc4c3F)c12] | 639.3 | 4 TFA salt, ¹H NMR (400 MHz, methanol-d₄, ppm): 7.68 (dd, J = 9.2, 6.0 Hz, 1H), 7.31 (d, J =2.4 Hz, 1H), 7.26 (t, J = 9.2 Hz, 1H), 7.08 (s, 1H), 5.66-5.52 (m, 1H), 4.82-4.33 (m, 4H), 4.26-3.81 (m, 7H), 3.50-3.43 (m, 1H), 2.82-2.54 (m, 2H), 2.52-2.29 (m, 5H), 2.26-1.78 (m, 5H), 0.90-0.80 (m, 3H). ¹⁹F NMR (376 MHz, methanol-d₄, ppm): δ −120.70 (1F), −141.53 (1F), −174.14 (1F). |
| 140 | 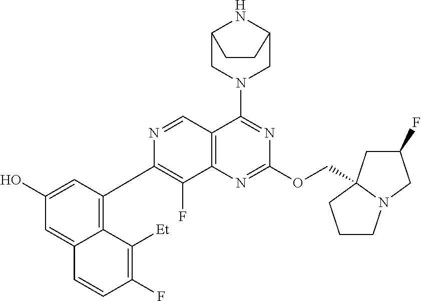 [SMILES: CCc1c(F)ccc2cc(O)cc(-c3ncc4c(N5CC6CCC(C5)N6)nc(OC[C@@]56CCCN5C[C@H](F)C6)nc4c3F)c12] | 605.3 | 3 TFA salt, ¹H NMR (400 MHz, methanol-d₄, ppm): δ 9.15 (s, 1H), 7.68 (dd, J = 9.2, 6.0 Hz, 1H), 7.31 (d, J = 2.8 Hz, 1H), 7.26 (t, J = 9.2 Hz, 1H), 7.03 (d, J = 2.4 Hz, 1H), 5.67- 5.51 (m, 1H), 4.98-4.88 (m, 2H), 4.77-4.70 (m, 2H), 4.32- 4.24 (m, 2H), 4.08-3.82 (m, 5H), 3.52-3.43 (m, 1H), 2.81- 2.55 (m, 2H), 2.51-2.30 (m, 4H), 2.28-2.05 (m, 6H), 0.82- 0.74 (m, 3H). ¹⁹F NMR (376 MHz, methanol-d₄, ppm): δ −121.09 (1F), −138.60 (1F), −174.10 (1F). |
| 141 | 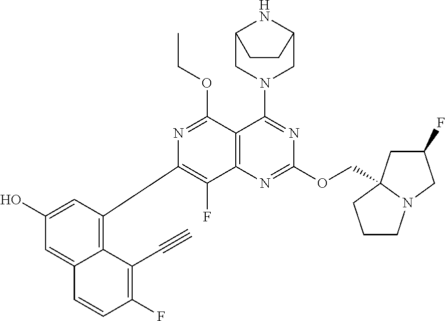 [SMILES: C#Cc1c(F)ccc2cc(O)cc(-c3nc(OCC)c4c(N5CC6CCC(C5)N6)nc(OC[C@@]56CCCN5C[C@H](F)C6)nc4c3F)c12] | 645.3 | ¹H NMR (400 MHz, methanol-d₄, ppm): δ 7.88-7.83 (m, 1H), 7.34-7.29 (m, 2H), 7.21-7.19 (m, 1H), 5.64-5.50 (m, 1H), 4.73-4.47 (m, 6H), 4.23-4.20 (m, 2H), 4.06-3.78 (m, 5H), 3.50-3.43 (m, 1H), 3.37-3.35 (m, 1H), 2.74-2.54 (m, 2H), 2.45-2.31 (m, 3H), 2.17-2.04 (m, 5H), 1.43 (t, J = 7.2 Hz, 3H). |
| 143 | 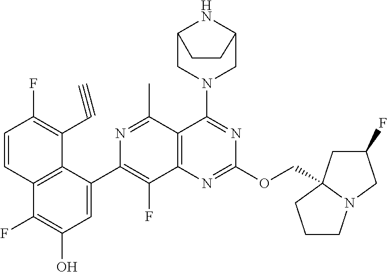 [SMILES: C#Cc1c(F)ccc2c(F)c(O)cc(-c3nc(C)c4c(N5CC6CCC(C5)N6)nc(OC[C@@]56CCCN5C[C@H](F)C6)nc4c3F)c12] | 633.3 | 3 TFA salt, ¹H NMR (400 MHz, methanol-d₄, ppm): δ 8.17 (dd, J = 9.2, 5.6 Hz, 1H), 7.47 (t, J = 9.2 Hz, 1H), 7.34 (d, J = 8.0 Hz, 1H), 5.64-5.51 (m, 1H), 4.72-4.48 (m, 4H), 4.22- 4.16 (m, 2H), 4.06-3.88 (m, 5H), 3.50-3.43 (m, 1H), 3.39- 3.38 (m, 1H), 2.78-2.55 (m, 5H), 2.47-2.31 (m, 3H), 2.23- 1.86 (m, 5H). ¹⁹F NMR (376 MHz, methanol-d₄, ppm): δ −110.24 (1F), −144.24 (1F), −147.98 (1F), −174.17 (1F). |
| 146 | 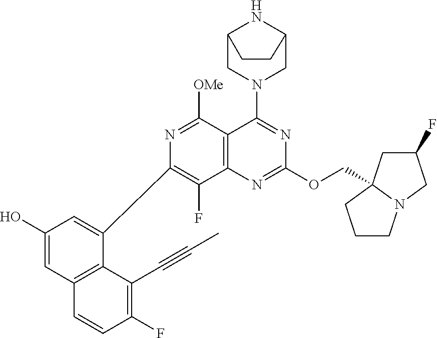 [SMILES: CC#Cc1c(F)ccc2cc(O)cc(-c3nc(OC)c4c(N5CC6CCC(C5)N6)nc(OC[C@@]56CCCN5C[C@H](F)C6)nc4c3F)c12] | 645.3 | 3 TFA salt, ¹H NMR (400 MHz, methanol-d₄, ppm): δ 7.79- 7.74 (m, 1H), 7.31-7.25 (m, 2H), 7.16 (s, 1H), 5.67-5.48 (m, 1H), 4.73-4.56 (m, 3H), 4.44-4.35 (m, 1H), 4.27-4.18 (m, 2H), 4.11 (s, 3H), 4.09-3.70 (m, 5H), 3.52-3.41 (m, 1H), 2.78-2.54 (m, 2H), 2.46-2.27 (m, 3H), 2.25-2.02 (m, 5H), 1.37 (s, 3H). ¹⁹F NMR (376 MHz, methanol-d₄, ppm): δ −113.41 (1F), −149.71 (1F), −174.26 (1F). |
| 147 | 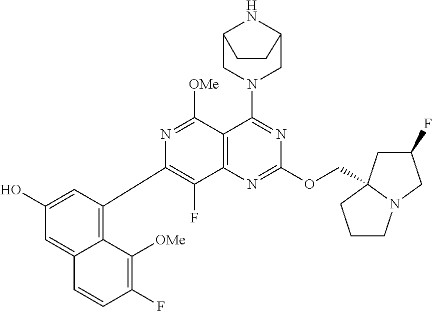 [SMILES: COc1c(F)ccc2cc(O)cc(-c3nc(OC)c4c(N5CC6CCC(C5)N6)nc(OC[C@@]56CCCN5C[C@H](F)C6)nc4c3F)c12] | 637.3 | 4 TFA salt, ¹H NMR (400 MHz, methanol-d₄, ppm): δ 7.52 (dd, J = 9.2, 5.2 Hz, 1H), 7.33-7.28 (m, 1H), 7.26 (d, J = 2.4 Hz, 1H), 7.10-7.06 (m, 1H), 5.68-5.43 (m, 1H), 4.74-4.62 (m, 2H), 4.57-4.41 (m, 2H), 4.27-4.16 (m, 2H), 4.08 (s, 3H), 4.05-3.82 (m, 3H), 3.81-3.70 (m, 2H), 3.53-3.44 (m, 1H), 3.43 (s, 3H), 2.82-2.51 (m, 2H), 2.48-2.26 (m, 3H), 2.25- 2.00 (m, 5H). ¹⁹F NMR (376 MHz, methanol-d₄, ppm): δ −137.90 (1F), −151.49 (1F), −174.24 (1F). |
| 148 | 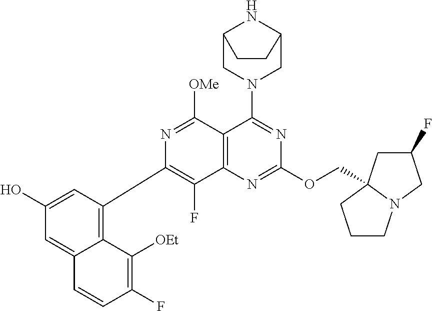 [SMILES: CCOc1c(F)ccc2cc(O)cc(-c3nc(OC)c4c(N5CC6CCC(C5)N6)nc(OC[C@@]56CCCN5C[C@H](F)C6)nc4c3F)c12] | 651.3 | 4 TFA salt, ¹H NMR (400 MHz, methanol-d₄, ppm): δ 7.52 (dd, J = 8.8, 4.8 Hz, 1H), 7.38-7.25 (m, 2H), 7.11-7.09 (m, 1H), 5.69-5.45 (m, 1H), 4.76-4.62 (m, 2H), 4.61-4.34 (m, 2H), 4.28-4.17 (m, 2H), 4.09 (s, 3H), 4.04-3.86 (m, 3H), 3.85-3.62 (m, 4H), 3.53-3.40 (m, 1H), 2.78-2.51 (m, 2H), 2.49-2.25 (m, 3H), 2.24-1.91 (m, 5H), 0.65-0.57 (m, 3H). ¹⁹F NMR (376 MHz, methanol-d₄, ppm): −137.19 (1F), −150.91 (1F), −174.18 (1F). |
| 149 | 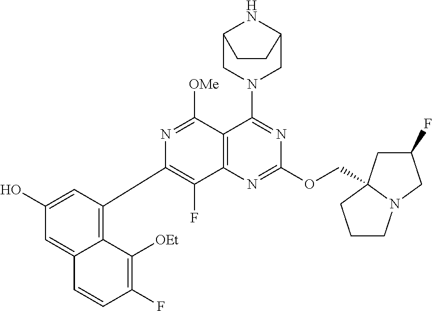 [SMILES: CCOc1c(F)ccc2cc(O)cc(-c3nc(OC)c4c(N5CC6CCC(C5)N6)nc(OC[C@@]56CCCN5C[C@H](F)C6)nc4c3F)c12] | 637.3 | 3 TFA salt, ¹H NMR (400 MHz, methanol-d₄, ppm): δ 7.52 (dd, J = 9.2, 4.8 Hz, 1H), 7.38-7.31 (m, 2H), 7.15 (d, J = 2.0 Hz, 1H), 5.66-5.50 (m, 1H), 4.73-4.60 (m, 2H), 4.59-4.35 (m, 2H), 4.26-4.13 (m, 2H), 4.10-3.85 (m, 5H), 3.71-3.56 (m, 2H), 3.53-3.40 (m, 1H), 2.80-2.49 (m, 2H), 2.47-2.29 (m, 3H), 2.28-2.00 (m, 5H), 0.92-0.84 (m, 3H). ¹⁹F NMR (376 MHz, methanol-d₄, ppm): −136.13 (1F), −164.36 (1F), −174.11 (1F). |
| 150 | 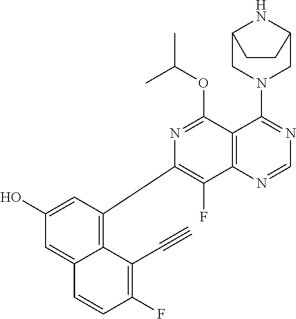 [SMILES: C#Cc1c(F)ccc2cc(O)cc(-c3nc(OC(C)C)c4c(N5CC6CCC(C5)N6)ncnc4c3F)c12] | 502.4 | ¹H NMR (400 MHz, methanol-d₄, ppm): δ 8.51 (s, 1H), 7.84 (dd, J = 9.2, 6.0 Hz, 1H), 7.45-7.27 (m, 2H), 7.22 (d, J = 2.4 Hz, 1H), 5.53-5.45 (m, 1H), 4.49-4.20 (m, 2H), 3.75-3.54 (m, 4H), 3.26 (s, 1H), 1.86-1.58 (m, 4H), 1.41-1.37 (m, 6H). ¹⁹F NMR (376 MHz, methanol-d₄, ppm): δ −111.84 (1F), −150.94 (1F). |
| 151 | 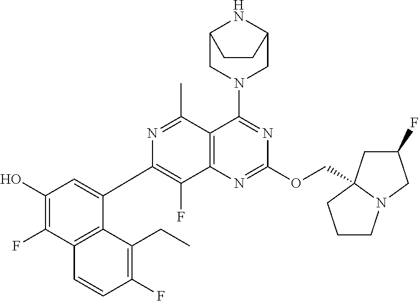 [SMILES: CCc1c(F)ccc2c(F)c(O)cc(-c3nc(C)c4c(N5CC6CCC(C5)N6)nc(OC[C@@]56CCCN5C[C@H](F)C6)nc4c3F)c12] | 637.4 | 3.0 TFA salt, ¹H NMR (400 MHz, methanol-d₄, ppm): δ 8.03-7.99 (m, 1H), 7.38 (t, J = 9.6 Hz, 1H), 7.21-7.18 (m, 1H), 5.66-5.52 (m, 1H), 4.74-4.17 (m, 6H), 4.07-3.84 (m, 5H), 3.50-3.44 (m, 1H), 2.78-1.79 (m, 15H), 0.80 (t, J = 7.2 Hz, 3H). ¹⁹F NMR (376 MHz, methanol-d₄, ppm): δ −119.68 (1F), −147.97 (1F), −144.21 (1F), −174.11(1F). |
| 154 | 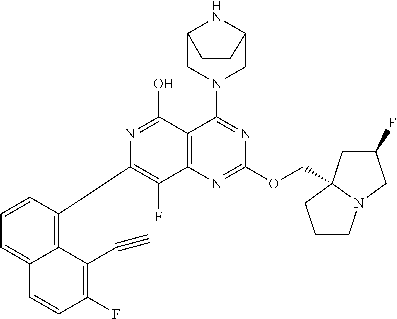 [SMILES: C#Cc1c(F)ccc2cccc(-c3nc(O)c4c(N5CC6CCC(C5)N6)nc(OC[C@@]56CCCN5C[C@H](F)C6)nc4c3F)c12] | 601.3 | 3.0 TFA salt, ¹H NMR (400 MHz, methanol-d₄, ppm): δ 8.19-8.12 (m, 2H), 7.72-7.66 (m, 2H), 7.49 (t, J = 8.8 Hz, 1H), 5.64-5.50 (m, 1H), 4.65-4.54 (m, 3H), 4.43-4.37 (m, 1H), 4.21-4.18 (m, 2H), 4.08-3.77 (m, 4H), 3.69-3.57 (m, 2H), 3.48-3.43 (m, 1H), 2.77-2.53 (m, 2H), 2.44-2.09 (m, 8H). ¹⁹F NMR (376 MHz, methanol-d₄, ppm): δ −105.82 (1F), −162.21 (1F), −174.10 (1F). |
| 157 | 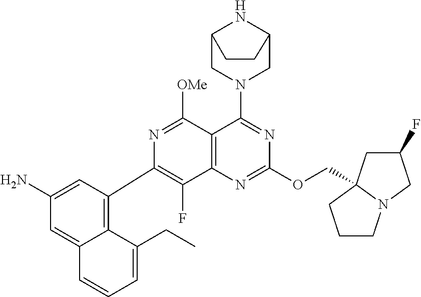 [SMILES: CCc1cccc2cc(N)cc(-c3nc(OC)c4c(N5CC6CCC(C5)N6)nc(OC[C@@]56CCCN5C[C@H](F)C6)nc4c3F)c12] | 616.4 | 3.0 TFA salt, ¹H NMR (400 MHz, methanol-d₄, ppm): δ 7.76 (d, J = 8.0 Hz, 1H), 7.67 (d, J = 2.4 Hz, 1H), 7.48 (t, J = 7.6 Hz, 1H), 7.32 (d, J = 6.8 Hz, 1H), 7.28-7.25 (m, 1H), 5.64- 5.51 (m, 1H), 4.72-4.65 (m, 2H), 4.52-4.43 (m, 2H), 4.23- 4.21 (m, 2H), 4.08-3.75 (m, 8H), 3.49-3.42 (m, 1H), 2.78- 2.32 (m, 7H), 2.28-2.04 (m, 5H), 0.95-0.91 (m, 3H). ¹⁹F NMR (376 MHz, methanol-d₄, ppm): δ −149.75 (1F), −174.09 (1F). |
| 158 | 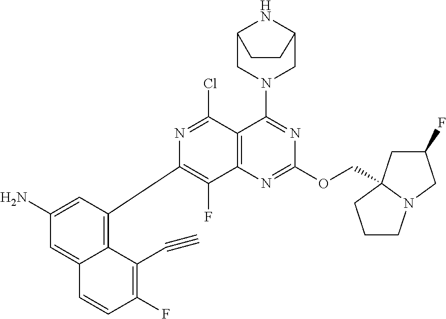 [SMILES: C#Cc1c(F)ccc2cc(N)cc(-c3nc(Cl)c4c(N5CC6CCC(C5)N6)nc(OC[C@@]56CCCN5C[C@H](F)C6)nc4c3F)c12] | 634.3 | 3.0 TFA salt, ¹H NMR (400 MHz, methanol-d₄, ppm): δ 7.80-7.76 (m, 1H), 7.29-7.24 (m, 2H), 7.19-7.17 (m, 1H), 5.65-5.52 (m, 1H), 4.96-4.93 (m, 1H), 4.76-4.38 (m, 4H), 4.27-4.18 (m, 2H), 4.13-3.84 (m, 4H), 3.52-3.44 (m, 1H), 3.35-3.34 (m, 1H), 2.80-2.54 (m, 2H), 2.50-1.71 (m, 8H). ¹⁹F NMR (376 MHz, methanol-d₄, ppm): δ −112.80 (1F), −141.45 (1F), −174.17 (1F). |
| 159 | 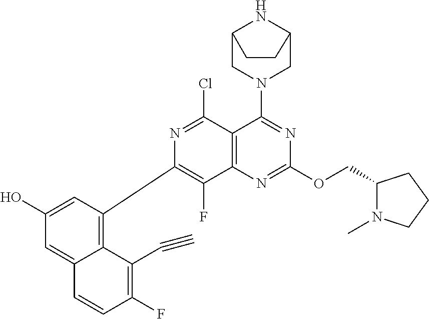 [SMILES: C#Cc1c(F)ccc2cc(O)cc(-c3nc(Cl)c4c(N5CC6CCC(C5)N6)nc(OC[C@@H]5CCCN5C)nc4c3F)c12] | 591.2 | 3.0 TFA salt, ¹H NMR (400 MHz, methanol-d₄, ppm): δ 7.90-7.86 (m, 1H), 7.37-7.32 (m, 2H), 7.24-7.23 (m, 1H), 4.95-4.87 (m, 1H), 4.75-4.41 (m, 3H), 4.24-4.19 (m, 2H), 4.15-3.70 (m, 4H), 3.44-3.42 (m, 1H), 3.31-3.28 (m, 1H), 3.14-3.08 (m, 3H), 2.46-2.36 (m, 1H), 2.32-1.75 (m, 7H). ¹⁹F NMR (376 MHz, methanol-d₄, ppm): δ −111.40 (1F), −141.45 (1F). |
| 160 | 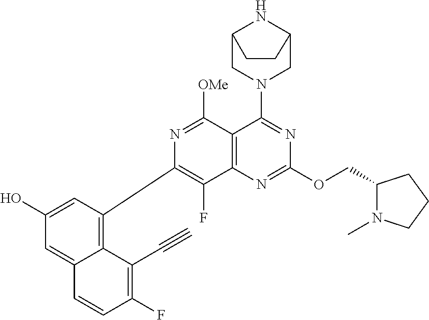 [SMILES: C#Cc1c(F)ccc2cc(O)cc(-c3nc(OC)c4c(N5CC6CCC(C5)N6)nc(OC[C@@H]5CCCN5C)nc4c3F)c12] | 587.3 | 3.0 TFA salt, ¹H NMR (400 MHz, methanol-d₄, ppm): δ 7.88-7.84 (m, 1H), 7.35-7.30 (m, 2H), 7.22 (s, 1H), 4.91- 4.87 (m, 1H), 4.70-4.65 (m, 1H), 4.47-4.44 (m, 2H), 4.23- 4.21 (m, 2H), 4.06 (s, 3H), 3.90-3.88 (m, 1H), 3.75-3.70 (m, 3H), 3.39-3.37 (m, 1H), 3.25-3.23 (m, 1H), 3.09 (s, 3H), 2.41-2.37 (m, 1H), 2.21-2.05 (m, 7H). ¹⁹F NMR (376 MHz, methanol-d₄, ppm): δ −111.82 (1F), −150.53 (1F). |
| 161 | 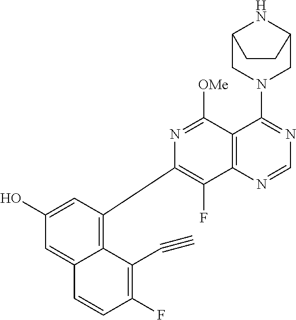 [SMILES: C#Cc1c(F)ccc2cc(O)cc(-c3nc(OC)c4c(N5CC6CCC(C5)N6)ncnc4c3F)c12] | 474.1 | 3.0 TFA salt, ¹H NMR (400 MHz, methanol-d₄, ppm): δ 8.72 (s, 1H), 7.88-7.85 (m, 1H), 7.35-7.31 (m, 2H), 7.25 (d, J = 2.4 Hz, 1H), 4.58-4.54 (m, 2H), 4.24-4.20 (m, 2H), 4.09 (s, 3H), 3.81-3.75 (m, 2H), 3.37 (s, 1H), 2.09-2.07 (m, 4H). ¹⁹F NMR (376 MHz, methanol-d₄, ppm): δ −111.74(1F), −149.42 (1F). |
| 162 | 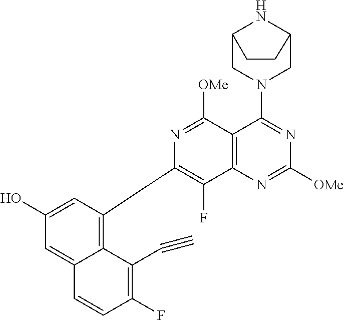 [SMILES: C#Cc1c(F)ccc2cc(O)cc(-c3nc(OC)c4c(N5CC6CCC(C5)N6)nc(OC)nc4c3F)c12] | 504.3 | 2.0 TFA salt, ¹H NMR (400 MHz, methanol-d₄, ppm): δ 7.85 (dd, J = 9.2, 6.0 Hz, 1H), 7.33-7.29 (m, 2H), 7.21 (d, J = 2.4 Hz, 1H), 4.48-4.39 (m, 2H), 4.24-4.18 (m, 2H), 4.07 (s, 3H), 4.04 (s, 3H), 3.71-3.65 (m, 2H), 3.37 (s, 1H), 2.19-2.09 (m, 4H). ¹⁹F NMR (376 MHz, methanol-d₄, ppm): δ −111.91 (1F), −150.13 (1F). |
| 163 | 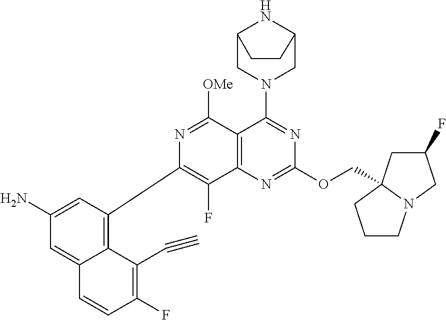 [SMILES: C#Cc1c(F)ccc2cc(N)cc(-c3nc(OC)c4c(N5CC6CCC(C5)N6)nc(OC[C@@]56CCCN5C[C@H](F)C6)nc4c3F)c12] | 630.2 | 3.0 TFA salt, ¹H NMR (400 MHz, methanol-d₄, ppm): δ 7.95-7.91 (m, 1H), 7.59 (s, 1H), 7.41-7.36 (m, 2H), 5.64- 5.51 (m, 1H), 4.74-4.64 (m, 2H), 4.51-4.42 (m, 2H), 4.26- 4.19 (m, 2H), 4.15-3.72 (m, 8H), 3.51-3.40 (m, 2H), 2.76- 2.54 (m, 2H), 2.45-2.30 (m, 3H), 2.26-2.04 (m, 5H). ¹⁹F NMR (376 MHz, methanol-d₄, ppm): δ −110.56 (1F), −150.47 (1F), −174.13 (1F). |
| 164 | 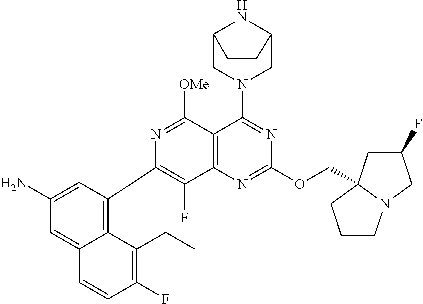 [SMILES: CCc1c(F)ccc2cc(N)cc(-c3nc(OC)c4c(N5CC6CCC(C5)N6)nc(OC[C@@]56CCCN5C[C@H](F)C6)nc4c3F)c12] | 634.4 | 4.0 TFA salt, ¹H NMR (400 MHz, methanol-d₄, ppm): δ 7.82-7.78 (m, 1H), 7.67-7.63 (m, 1H), 7.35 (t, J = 9.2 Hz, 1H), 7.28-7.26 (m, 1H), 5.64-5.51 (m, 1H), 4.71-4.67 (m, 2H), 4.54-4.41 (m, 2H), 4.26-4.19 (m, 2H), 4.09-3.72 (m, 8H), 3.49-3.42 (m, 1H), 2.79-2.52 (m, 3H), 2.49-2.29 (m, 4H), 2.27-2.01 (m, 5H), 0.86-0.82 (m, 3H). ¹⁹F NMR (376 MHz, methanol-d₄, ppm): δ −118.22 (1F), −149.40 (1F), −174.12 (1F). |
| 166 | 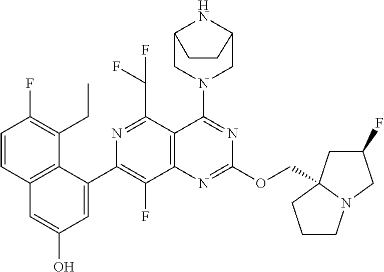 [SMILES: CCc1c(F)ccc2cc(O)cc(-c3nc(C(F)F)c4c(N5CC6CCC(C5)N6)nc(OC[C@@]56CCCN5C[C@H](F)C6)nc4c3F)c12] | 655.3 | 3.0 TFA salt, ¹H NMR (400 MHz, methanol-d₄, ppm): δ 7.69 (dd, J = 9.2, 6.0 Hz, 1H), 7.33 (d, J = 2.4 Hz, 1H), 7.31-6.89 (m, 3H), 5.70-5.44 (m, 1H), 4.83-4.58 (m, 4H), 4.33-4.11 (m, 2H), 4.09-3.82 (m, 5H), 3.54-3.40 (m, 1H), 2.86-2.52 (m, 2H), 2.51-2.15 (m, 6H), 2.13-1.73 (m, 4H), 0.82-0.72 (m, 3H). ¹⁹F NMR (376 MHz, methanol-d₄, ppm): δ −82.28 (2 F), −120.85 (1 F), −136.76 (1F), −174.17 (1F). |
| 167 | 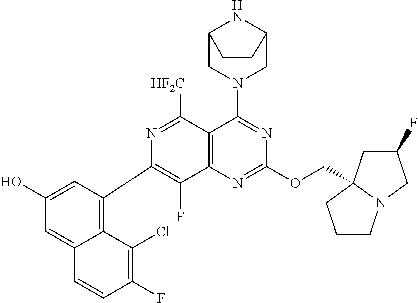 | 661.3 | 3.0 TFA salt, ¹H NMR (400 MHz, methanol-d₄, ppm): δ 7.85-7.81 (m, 1H), 7.43-7.38 (m, 2H), 7.29-6.97 (m, 2H), 5.65-5.52 (m, 1H), 4.81-4.49 (m, 4H), 4.22-4.18 (m, 2H), 4.05-3.84 (m, 5H), 3.50-3.43 (m, 1H), 2.79-2.55 (m, 2H), 2.47-2.31 (m, 3H), 2.25-1.72 (m, 5H). ¹⁹F NMR (376 MHz, methanol-d₄, ppm): δ −82.51 (2F), −116.55 (1F), −137.54 (1F), −174.16 (1F). |
| 168 | 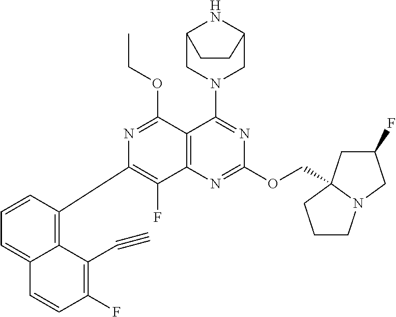 [SMILES: C#Cc1c(F)ccc2cccc(-c3nc(OCC)c4c(N5CC6CCC(C5)N6)nc(OC[C@@]56CCCN5C[C@H](F)C6)nc4c3F)c12] | 629.3 | 3.0 TFA salt, ¹H NMR (400 MHz, methanol-d₄, ppm): δ 8.12-8.07 (m, 2H), 7.66-7.63 (m, 2H), 7.44 (t, J = 9.2 Hz, 1H), 5.64-5.50 (m, 1H), 4.72-4.65 (m, 2H), 4.61-4.46 (m, 4H), 4.23-4.20 (m, 2H), 4.07-3.79 (m, 5H), 3.49-3.42 (m, 2H), 2.76-2.54 (m, 2H), 2.46-2.30 (m, 3H), 2.25-2.00 (m, 5H), 1.43 (t, J = 7.2 Hz, 3H). ¹⁹F NMR (376 MHz, methanol-d₄, ppm): δ −107.01 (1F), −151.07 (1F), −174.17 (1F). |
| 169 | 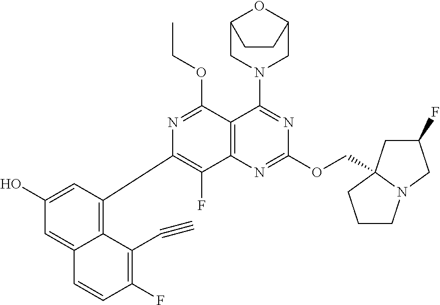 [SMILES: C#Cc1c(F)ccc2cc(O)cc(-c3nc(OCC)c4c(N5CC6CCC(C5)O6)nc(OC[C@@]56CCCN5C[C@H](F)C6)nc4c3F)c12] | 646.2 | 4.0 TFA salt, ¹H NMR (400 MHz, methanol-d₄, ppm): δ 7.87-7.83 (m, 1H), 7.34-7.29 (m, 2H), 7.21 (d, J = 2.4 Hz, 1H), 5.63-5.50 (m, 1H), 4.71-4.42 (m, 6H), 4.38-4.12 (m, 2H), 4.10-3.79 (m, 3H), 3.77-3.59 (m, 2H), 3.50-3.40 (m, 1H), 3.36-3.34 (m, 1H), 2.78-2.51 (m, 2H), 2.50-2.27 (m, 3H), 2.23-2.07 (m, 1H), 1.98-1.65 (m, 4H), 1.42 (t, J = 7.2 Hz, 3H). ¹⁹F NMR (376 MHz, methanol-d₄, ppm): δ −111.77 (1F), −151.91 (1F), −174.18 (1F). |
| 172 | 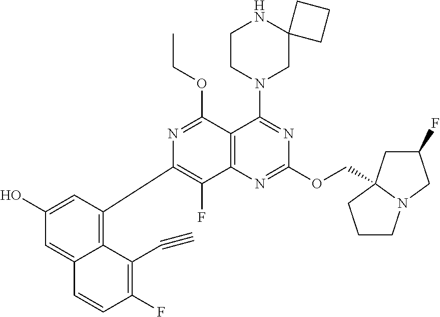 [SMILES: C#Cc1c(F)ccc2cc(O)cc(-c3nc(OCC)c4c(N5CCNC6(CCC6)C5)nc(OC[C@@]56CCCN5C[C@H](F)C6)nc4c3F)c12] | 659.4 | ¹H NMR (400 MHz, methanol-d₄, ppm): δ7.83 (dd, J = 9.2, 6.0 Hz, 1H), 7.32-7.27 (m, 2H), 7.21 (s, 1H), 5.36-5.23 (m, 1H), 4.56-4.43 (m, 2H), 4.29-4.19 (m, 2H), 3.92-3.82 (m, 3H), 3.63-3.59 (m, 1H), 3.25-3.17 (m, 3H), 3.01-2.88 (m, 3H), 2.33-1.79 (m, 13H), 1.41 (t, J = 7.2 Hz, 3H). |
| 173 | 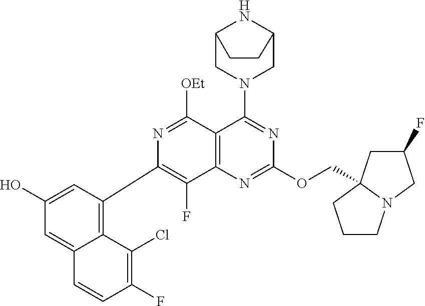 [SMILES: CCOc1nc(-c2cc(O)cc3ccc(F)c(Cl)c23)c(F)c2nc(OC[C@@]34CCCN3C[C@H](F)C4)nc(N3CC4CCC(C3)N4)c12] | 655.3 | 3.0 TFA salt, ¹H NMR (400 MHz, methanol-d₄, ppm): δ 7.80 (dd, J = 9.2, 5.2 Hz, 1H), 7.39 (t, J = 8.8 Hz, 1H), 7.34 (d, J = 2.4 Hz, 1H), 7.21-7.20 (m, 1H), 5.64-5.51 (m, 1H), 4.71- 4.64 (m, 2H), 4.58-4.48 (m, 4H), 4.22-4.19 (m, 2H), 4.16- 3.80 (m, 5H), 3.50-3.43 (m, 1H), 2.77-2.54 (m, 2H), 2.45- 2.30 (m, 3H), 2.21-2.02 (m, 5H), 1.43 (t, J = 6.8 Hz, 3H). ¹⁹F NMR (376 MHz, methanol-d₄, ppm): δ −116.88 (1F), −151.17 (1F), −174.17 (1F). |
| 174 | 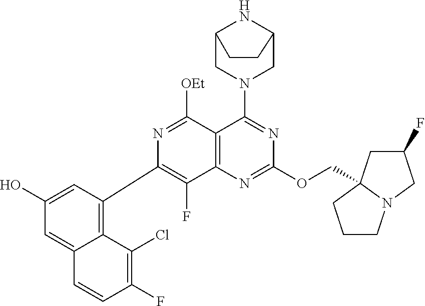 [SMILES: CCOc1nc(-c2cc(O)cc3ccc(F)c(Cl)c23)c(F)c2nc(OC[C@@]34CCCN3C[C@H](F)C4)nc(N3CC4CCC(C3)N4)c12] | 627.3 | 3.0 TFA salt, ¹H NMR (400 MHz, methanol-d₄, ppm): δ 7.83 (dd, J = 9.2, 5.6 Hz, 1H), 7.46-7.42 (m, 2H), 7.30 (d, J = 2.4 Hz, 1H), 5.64-5.51 (m, 1H), 4.69-4.53 (m, 3H), 4.44-4.37 (m, 1H), 4.23-4.15 (m, 2H), 4.09-3.83 (m, 3H), 3.69-3.57 (m, 2H), 3.49-3.43 (m, 1H), 2.77-2.53 (m, 2H), 2.44-2.03 (m, 8H). ¹⁹F NMR (376 MHz, methanol-d₄, ppm): δ −115.88 (1F), −162.48 (1F), −174.17 (1F). |
| 175 | 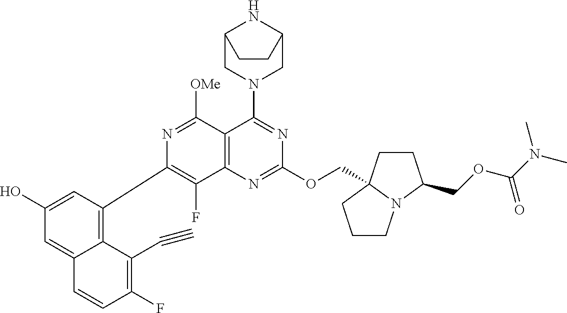 [SMILES: C#Cc1c(F)ccc2cc(O)cc(-c3nc(OC)c4c(N5CC6CCC(C5)N6)nc(OC[C@@]56CCCN5[C@H](COC(=O)N(C)C)CC6)nc4c3F)c12] | 714.4 | 3.0 TFA salt, ¹H NMR (400 MHz, methanol-d₄, ppm): δ 7.88-7.83 (m, 1H), 7.35-7.29 (m, 2H), 7.24-7.21 (m, 1H), 4.75-4.69 (m, 1H), 4.67-4.59 (m, 1H), 4.54-4.41 (m, 3H), 4.39-4.31 (m, 1H), 4.30-4.19 (m, 3H), 4.08-4.04 (m, 3H), 3.78-3.69 (m, 2H), 3.64-3.56 (m, 1H), 3.48-3.37 (m, 1H), 3.35-3.33 (m, 1H), 2.94-2.83 (m, 6H), 2.46-2.30 (m, 2H), 2.28-2.19 (m, 2H), 2.18-2.05 (m, 8H). ¹⁹F NMR (376 MHz, methanol-d₄, ppm): δ −111.77 (1F), −150.29 (1F). |
| 177 | 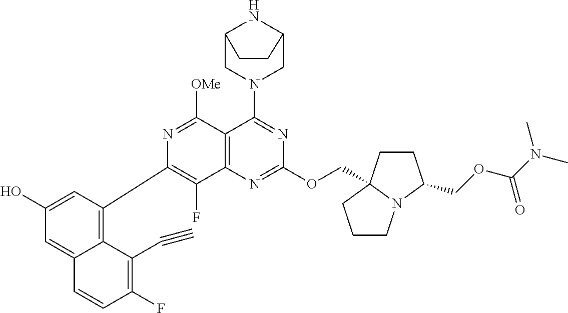 [SMILES: C#Cc1c(F)ccc2cc(O)cc(-c3nc(OC)c4c(N5CC6CCC(C5)N6)nc(OC[C@@]56CCCN5[C@@H](COC(=O)N(C)C)CC6)nc4c3F)c12] | 714.4 | 3.0 TFA salt, ¹H NMR (400 MHz, methanol-d₄, ppm): δ 7.88-7.83 (m, 1H), 7.35-7.29 (m, 2H), 7.24-7.21 (m, 1H), 4.75-4.64 (m, 2H), 4.54-4.41 (m, 3H), 4.34-4.21 (m, 3H), 4.08-4.04 (m, 3H), 3.91-3.84 (m, 1H), 3.78-3.60 (m, 3H), 3.59-3.45 (m, 1H), 3.39-3.34 (m, 1H), 2.89-2.75 (m, 6H), 2.54-2.44 (m, 1H), 2.32-2.08 (m, 11H). ¹⁹F NMR (376 MHz, methanol-d₄, ppm): δ −111.74 (1F), −150.31 (1F). |
| 180 | 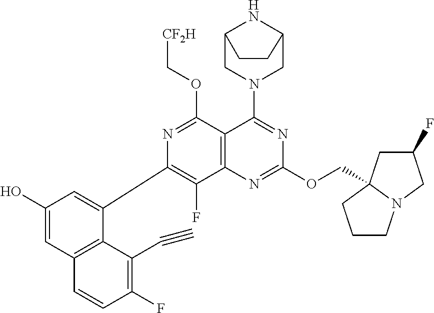 [SMILES: C#Cc1c(F)ccc2cc(O)cc(-c3nc(OCC(F)F)c4c(N5CC6CCC(C5)N6)nc(OC[C@@]56CCCN5C[C@H](F)C6)nc4c3F)c12] | 681.4 | 3.0 TFA salt, ¹H NMR (400 MHz, methanol-d₄, ppm): δ 7.87 (dd, J = 9.2, 6.0 Hz, 1H), 7.38-7.28 (m, 2H), 7.22 (s, 1H), 6.42-6.11 (m, 1H), 5.70-5.46 (m, 1H), 4.81-4.40 (m, 6H), 4.26-4.15 (m, 2H), 4.09-3.73 (m, 5H), 3.56-3.43 (m, 1H), 3.42-3.38 (m, 1H), 2.79-2.51 (m, 2H), 2.49-2.29 (m, 3H), 2.26-1.94 (m, 5H). ¹⁹F NMR (376 MHz, methanol-d₄, ppm): δ −111.59 (1 F), −127.15 (2 F), −148.99 (1 F), −174.23 (1 F). |
| 181 | 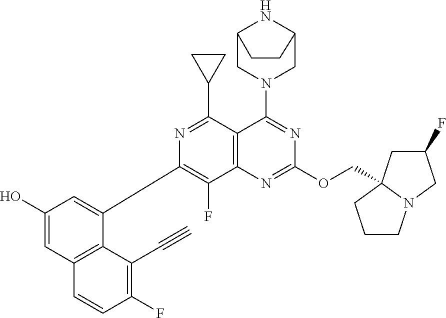 [SMILES: C#Cc1c(F)ccc2cc(O)cc(-c3nc(C4CC4)c4c(N5CC6CCC(C5)N6)nc(OC[C@@]56CCCN5C[C@H](F)C6)nc4c3F)c12] | 641.3 | 3.0 TFA salt, ¹H NMR (400 MHz, methanol-d₄, ppm): δ 7.87-7.83 (m, 1H), 7.33-7.29 (m, 2H), 7.16 (s, 1H), 5.64- 5.51 (m, 1H), 4.85-4.75 (m, 2H), 4.75-4.65 (m, 2H), 4.41- 3.83 (m, 7H), 3.50-3.43 (m, 1H), 3.23-3.22 (m, 1H), 2.77- 2.55 (m, 2H), 2.51-1.52 (m, 9H), 1.49-1.12 (m, 4H). ¹⁹F NMR (376 MHz, methanol-d₄, ppm): δ −111.87 (1F), −146.59 (1F), −174.17 (1F). |
| 182 | 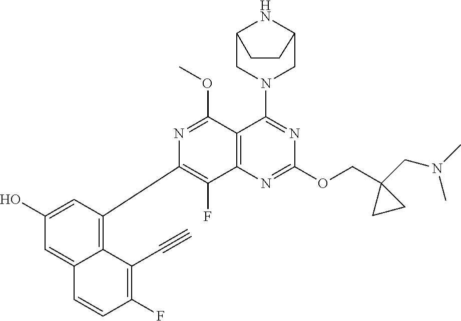 [SMILES: C#Cc1c(F)ccc2cc(O)cc(-c3nc(OC)c4c(N5CC6CCC(C5)N6)nc(OCC5(CN(C)C)CC5)nc4c3F)c12] | 601.3 | 4.0 TFA salt, ¹H NMR (400 MHz, methanol-d₄, ppm): δ 7.89-7.82 (m, 1H), 7.37-7.29 (m, 2H), 7.23 (d, J = 2.4 Hz, 1H), 4.54-4.36 (m, 4H), 4.26-4.17 (m, 2H), 4.06 (s, 3H), 3.77-3.68 (m, 2H), 3.41-3.34 (m, 2H), 3.29-3.22 (m, 1H), 2.98 (s, 6H), 2.22-2.04 (m, 4H), 1.02-0.83 (m, 4H). ¹⁹F NMR (376 MHz, methanol-d₄, ppm): δ −111.74 (1F), −151.17 (1F). |
| 184 | 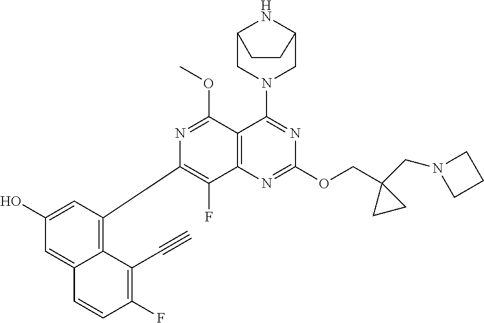 [SMILES: C#Cc1c(F)ccc2cc(O)cc(-c3nc(OC)c4c(N5CC6CCC(C5)N6)nc(OCC5(CN6CCC6)CC5)nc4c3F)c12] | 613.4 | 4.0 TFA salt, ¹H NMR (400 MHz, methanol-d₄, ppm): δ 7.90-7.82 (m, 1H), 7.40-7.27 (m, 2H), 7.27-7.13 (m, 1H), 4.49-4.28 (m, 6H), 4.27-4.12 (m, 4H), 4.06 (s, 3H), 3.81- 3.64 (m, 2H), 3.41-3.32 (m, 3H), 2.70-2.54 (m, 1H), 2.42- 2.28 (m, 1H), 2.21-2.03 (m, 4H), 0.95-0.78 (m, 4H). ¹⁹F NMR (376 MHz, methanol-d₄, ppm): δ −111.8 (1F), −150.5 (1F). |
| 186 | 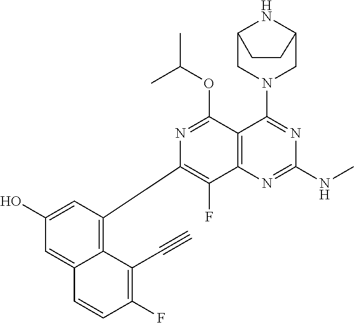 [SMILES: C#Cc1c(F)ccc2cc(O)cc(-c3nc(OC(C)C)c4c(N5CC6CCC(C5)N6)nc(NC)nc4c3F)c12] | 531.3 | 3.0 TFA salt, ¹H NMR (400 MHz, methanol-d₄, ppm): δ 7.89-7.82 (m, 1H), 7.36-7.30 (m, 2H), 7.23-7.20 (m, 1H), 5.60-5.48 (m, 1H), 4.82-4.40 (m, 2H), 4.35-4.20 (m, 2H), 4.04-3.86 (m, 2H), 3.65 (s, 1H), 3.12 (s, 3H), 2.20-1.97 (m, 4H), 1.48-1.36 (m, 6H). ¹⁹F NMR (376 MHz, methanol-d₄, ppm): δ −111.51 (1F), −153.65 (1F). |
| 187 | 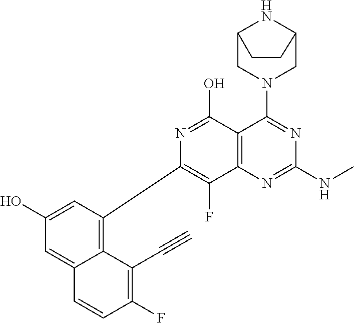 [SMILES: C#Cc1c(F)ccc2cc(O)cc(-c3nc(O)c4c(N5CC6CCC(C5)N6)nc(NC)nc4c3F)c12] | 489.3 | 3.0 TFA salt, ¹H NMR (400 MHz, methanol-d₄, ppm): δ 7.93-7.84 (m, 1H), 7.44-7.33 (m, 2H), 7.31-7.25 (m, 1H), 4.66-4.12 (m, 4H), 3.96-3.84 (m, 1H), 3.75-3.56 (m, 2H), 3.07 (s, 3H), 2.47-2.24 (m, 2H), 2.17-2.01 (m, 2H). ¹⁹F NMR (376 MHz, methanol-d₄, ppm): δ −110.57 (1F), −164.48 (1F) |
| 188 | 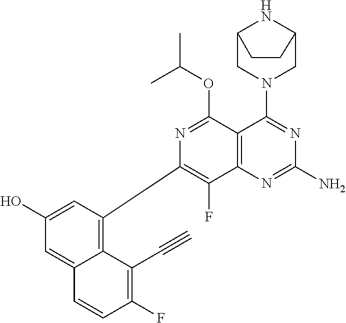 [SMILES: C#Cc1c(F)ccc2cc(O)cc(-c3nc(OC(C)C)c4c(N5CC6CCC(C5)N6)nc(N)nc4c3F)c12] | 517.3 | 3.0 TFA salt, ¹H NMR (400 MHz, methanol-d₄, ppm): δ 7.90-7.84 (m, 1H), 7.39-7.32 (m, 2H), 7.23-7.21 (m, 1H), 5.60-5.48 (m, 1H), 4.85-4.35 (m, 2H), 4.32-4.21 (m, 2H), 4.00-3.78 (m, 2H), 3.66 (s, 1H), 2.13-1.91 (m, 4H), 1.47- 1.36 (m, 6H). ¹⁹F NMR (376 MHz, methanol-d₄, ppm): δ −111.51 (1F), −153.65 (1F). |
| 189 | 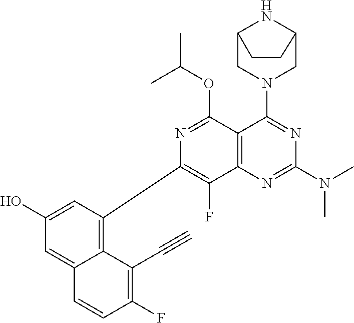 [SMILES: C#Cc1c(F)ccc2cc(O)cc(-c3nc(OC(C)C)c4c(N5CC6CCC(C5)N6)nc(N(C)C)nc4c3F)c12] | 545.3 | 3.0 TFA salt, ¹H NMR (400 MHz, methanol-d₄, ppm): δ 7.88-7.82 (m, 1H), 7.36-7.28 (m, 2H), 7.24-7.17 (m, 1H), 5.58-5.47 (m, 1H), 4.66-4.32 (m, 2H), 4.27-4.17 (m, 2H), 3.92-3.75 (m, 2H), 3.53 (s, 1H), 3.33 (s, 6H), 2.17-1.90 (m, 4H), 1.43-1.36 (m, 6H). ¹⁹F NMR (376 MHz, methanol-d₄, ppm): δ −111.86 (1F), −151.84 (1F). |
| 191 | 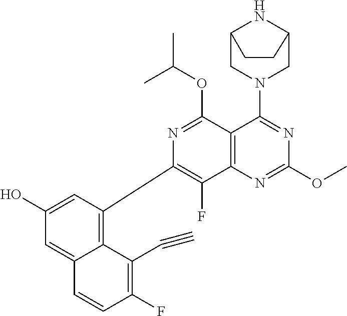 [SMILES: C#Cc1c(F)ccc2cc(O)cc(-c3nc(OC(C)C)c4c(N5CC6CCC(C5)N6)nc(OC)nc4c3F)c12] | 532.3 | 2.0 TFA salt, ¹H NMR (400 MHz, methanol-d₄, ppm): δ 7.86-7.82 (m, 1H), 7.33-7.28 (m, 2H), 7.19-7.18 (m, 1H), 5.56-5.50 (m, 1H), 4.58-4.49 (m, 2H), 4.22-4.19 (m, 2H), 4.09 (s, 3H), 3.89-3.83 (m, 2H), 3.40 (s, 1H), 2.04-2.01 (m, 4H), 1.41 (d, J = 6.0 Hz, 6H). ¹⁹F NMR (376 MHz, methanol-d₄, ppm): δ −111.92 (1F), 151.05 (1F). |
| 192 | 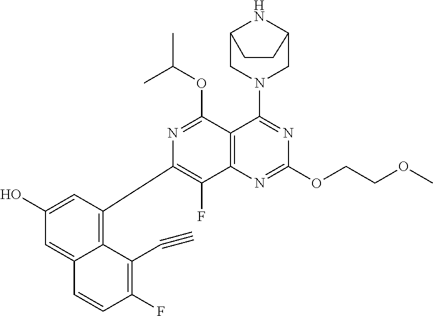 [SMILES: C#Cc1c(F)ccc2cc(O)cc(-c3nc(OC(C)C)c4c(N5CC6CCC(C5)N6)nc(OCCOC)nc4c3F)c12] | 576.3 | 2.0 TFA salt, ¹H NMR (400 MHz, methanol-d₄, ppm): δ 7.84 (dd, J = 9.2, 6.0 Hz, 1H), 7.33-7.28 (m, 2H), 7.18 (d, J = 2.4 Hz, 1H), 5.54-5.51 (m, 1H), 4.63-4.60 (m, 2H), 4.57-4.46 (m, 2H), 4.20-4.17 (m, 2H), 3.85-3.77 (m, 4H), 3.42 (s, 3H), 3.39 (s, 1H), 2.06-2.03 (m, 4H), 1.41 (d, J = 6.4 Hz, 6H). ¹⁹F NMR (376 MHz, methanol-d₄, ppm): δ −111.97 (1F), −150.92 (1F). |
| 193 | 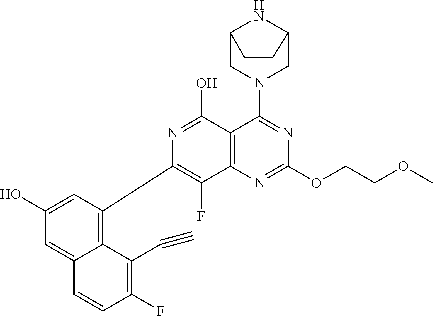 [SMILES: C#Cc1c(F)ccc2cc(O)cc(-c3nc(O)c4c(N5CC6CCC(C5)N6)nc(OCCOC)nc4c3F)c12] | 534.3 | 3.0 TFA salt, ¹H NMR (400 MHz, methanol-d₄, ppm): δ7.87 (dd, J = 9.2, 5.6 Hz, 1H), 7.38-7.33 (m, 2H), 7.27 (d, J = 2.4 Hz, 1H), 4.60-4.58 (m, 2H), 4.50-4.38 (m, 2H), 4.20-4.16 (m, 2H), 3.77 (t, J = 4.8 Hz, 2H), 3.72 (s, 1H), 3.62-3.53 (m, 2H), 3.41 (s, 3H), 2.34-2.31 (m, 2H), 2.08-2.06 (m, 2H). ¹⁹F NMR (376 MHz, methanol-d₄, ppm): δ −110.85 (1F), −161.49 (1F). |
| 194 | 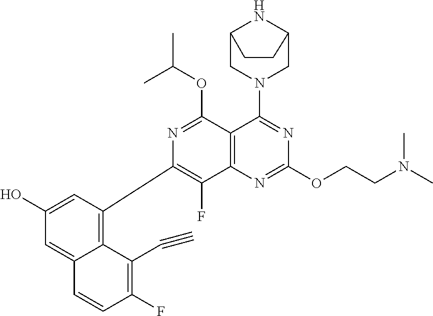 [SMILES: C#Cc1c(F)ccc2cc(O)cc(-c3nc(OC(C)C)c4c(N5CC6CCC(C5)N6)nc(OCCN(C)C)nc4c3F)c12] | 589.4 | 3.0 TF A salt, ¹H NMR (400 MHz, methanol-d₄, ppm): δ 7.85 (dd, J = 9.2, 6.0 Hz, 1H), 7.34-7.29 (m, 2H), 7.18 (d, J = 2.4 Hz, 1H), 5.56-5.26 (m, 1H), 4.82-4.79 (m, 2H), 4.57-4.48 (m, 2H), 4.22-4.18 (m, 2H), 3.87-3.82 (m, 2H), 3.64-3.61 (m, 2H), 3.38 (s, 1H), 3.01 (s, 6H), 2.06-1.99 (m, 4H), 1.41 (d, J = 6.0 Hz, 6H). ¹⁹F NMR (376 MHz, methanol-d₄, ppm): δ −111.91 (1F), −151.37 (1F). |
| 195 | 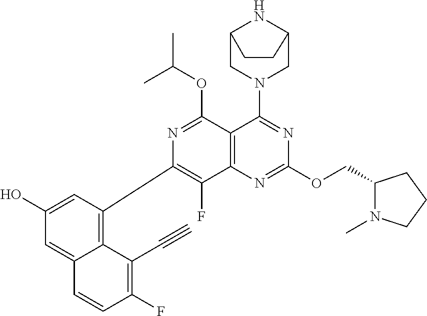 [SMILES: C#Cc1c(F)ccc2cc(O)cc(-c3nc(OC(C)C)c4c(N5CC6CCC(C5)N6)nc(OC[C@@H]5CCCN5C)nc4c3F)c12] | 615.3 | 3.0 TFA salt, ¹H NMR (400 MHz, methanol-d₄, ppm): δ 7.89-7.82 (m, 1H), 7.36-7.28 (m, 2H), 7.22-7.16 (m, 1H), 5.60-5.49 (m, 1H), 4.86-4.81 (m, 1H), 4.72-4.62 (m, 1H), 4.61-4.45 (m, 2H), 4.26-4.16 (m, 2H), 3.95-3.82 (m, 3H), 3.79-3.65 (m, 1H), 3.42-3.36 (m, 1H), 3.28-3.18 (m, 1H), 3.13-3.02 (m, 3H), 2.45-2.33 (m, 1H), 2.26-2.16 (m, 1H), 2.13-1.93 (m, 6H), 1.44-1.37 (m, 6H). ¹⁹F NMR (376 MHz, methanol-d₄, ppm): δ −111.88 (1F), −151.35 (1F). |
| 196 | 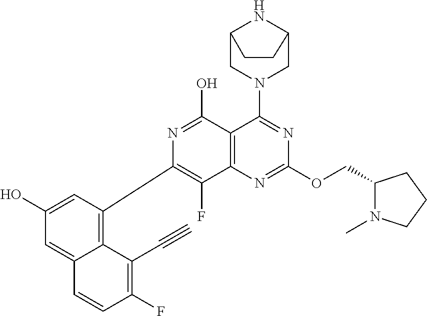 [SMILES: C#Cc1c(F)ccc2cc(O)cc(-c3nc(O)c4c(N5CC6CCC(C5)N6)nc(OC[C@@H]5CCCN5C)nc4c3F)c12] | 573.3 | 3.0 TFA salt, ¹H NMR (400 MHz, methanol-d₄, ppm): δ 7.93-7.85 (m, 1H), 7.42-7.33 (m, 2H), 7.32-7.23 (m, 1H), 4.85-4.81 (m, 1H), 4.70-4.61 (m, 1H), 4.58-4.48 (m, 1H), 4.46-4.35 (m, 1H), 4.25-4.13 (m, 2H), 3.93-3.83 (m, 1H), 3.78-3.68 (m, 2H), 3.68-3.54 (m, 2H), 3.27-3.19 (m, 1H), 3.11-3.00 (m, 3H), 2.45-2.00 (m, 8H). ¹⁹F NMR (376 MHz, methanol-d₄, ppm): δ −111.77 (1F), −162.15 (1F). |
| 197 | 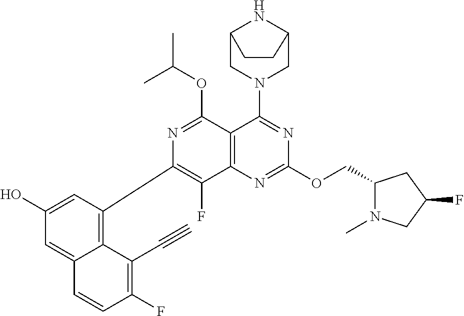 [SMILES: C#Cc1c(F)ccc2cc(O)cc(-c3nc(OC(C)C)c4c(N5CC6CCC(C5)N6)nc(OC[C@@H]5C[C@@H](F)CN5C)nc4c3F)c12] | 633.3 | 4.0 TFA salt, ¹H NMR (400 MHz, methanol-d₄, ppm): δ 7.89-7.82 (m, 1H), 7.36-7.27 (m, 2H), 7.24-7.15 (m, 1H), 5.59-5.38 (m, 2H), 5.00-4.93 (m, 1H), 4.76-4.69 (m, 1H), 4.60-4.44 (m, 2H), 4.29-4.16 (m, 3H), 4.14-3.95 (m, 1H), 3.94-3.79 (m, 2H), 3.75-3.56 (m, 1H), 3.43-3.35 (m, 1H), 3.20-3.12 (m, 3H), 2.73-2.61 (m, 1H), 2.50-2.30 (m, 1H), 2.12-1.96 (m, 4H),1.48-1.36 (m, 6H). ¹⁹F NMR (376 MHz, methanol-d₄, ppm): δ −111.86 (1F), −151.48 (1F), −174.23 (1F). |
| 200 | 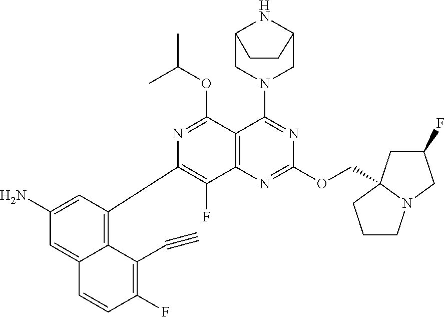 [SMILES: C#Cc1c(F)ccc2cc(N)cc(-c3nc(OC(C)C)c4c(N5CC6CCC(C5)N6)nc(OC[C@@]56CCCN5C[C@H](F)C6)nc4c3F)c12] | 658.4 | 3.0 TFA salt, ¹H NMR (400 MHz, methanol-d₄, ppm): δ 7.86 (dd, J = 9.2, 6.0 Hz, 1H), 7.42 (s, 1H), 7.33 (t, J = 8.8 Hz, 1H), 7.27-7.25 (m, 1H), 5.64-5.50 (m, 2H), 4.72-4.64 (m, 2H), 4.58-4.48 (m, 2H), 4.23-4.19 (m, 2H), 4.07-3.83 (m, 5H), 3.49-3.43 (m, 1H), 3.39-3.37 (m, 1H) 2.75-2.54 (m, 2H), 2.45-2.30 (m, 3H), 2.17-1.99 (m, 5H), 1.42-1.40 (m, 6H). ¹⁹F NMR (376 MHz, methanol-d₄, ppm): δ −111.70 (1F), −151.41 (1F), −174.14 (1F). |
| 201 | 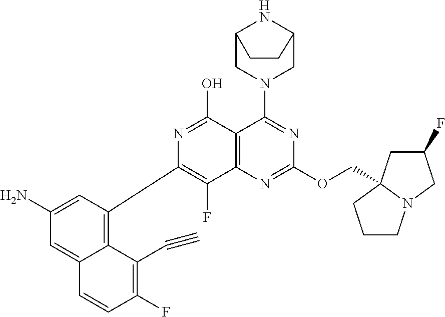 [SMILES: C#Cc1c(F)ccc2cc(N)cc(-c3nc(O)c4c(N5CC6CCC(C5)N6)nc(OC[C@@]56CCCN5C[C@H](F)C6)nc4c3F)c12] | 616.4 | 3.0 TF A salt, ¹H NMR (400 MHz, methanol-d₄, ppm): δ 7.78 (dd, J = 9.2, 5.6 Hz, 1H), 7.322-7.20 (m, 3H), 5.64-5.50 (m, 1H), 4.70-4.38 (m, 4H), 4.24-4.16 (m, 2H), 4.08-3.83 (m, 3H), 3.67-3.57 (m, 3H), 3.49-3.43 (m, 1H), 2.76-2.53 (m, 2H), 2.45-2.26 (m, 5H), 2.21-2.03 (m, 3H). ¹⁹F NMR (376 MHz, methanol-d₄, ppm): δ −112.35 (1F), −162.30 (1F), −174.14 (1F). |
| 206 | 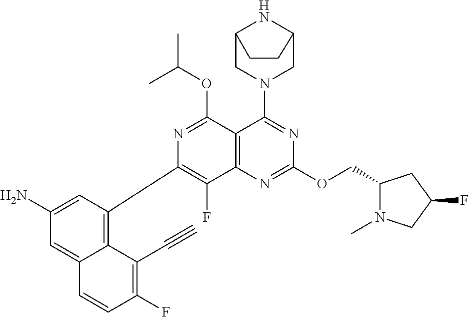 [SMILES: C#Cc1c(F)ccc2cc(N)cc(-c3nc(OC(C)C)c4c(N5CC6CCC(C5)N6)nc(OC[C@@H]5C[C@@H](F)CN5C)nc4c3F)c12] | 632.4 | 4.0 TFA salt, ¹H NMR (400 MHz, methanol-d₄, ppm): δ 7.91-7.86 (m, 1H), 7.53-7.49 (m, 1H), 7.37-7.31 (m, 2H), 5.58-5.39 (m, 2H), 4.99-4.93 (m, 1H), 4.76-4.72 (m, 1H), 4.58-4.45 (m, 2H), 4.31-3.98 (m, 4H), 3.93-3.84 (m, 2H), 3.73-3.56 (m, 1H), 3.44-3.41 (m, 1H), 3.18 (s, 3H), 2.73- 2.60 (m, 1H), 2.52-2.30 (m, 1H), 2.12-1.96 (m, 4H), 1.42- 1.38 (m, 6H). ¹⁹F NMR (376 MHz, methanol-d₄, ppm): δ −111.44 (1F), −151.33 (1F), −173.47 (1F). |
| 207 | 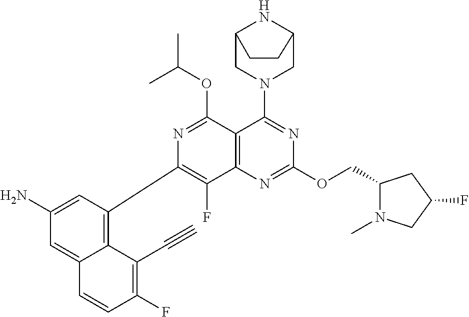 [SMILES: C#Cc1c(F)ccc2cc(N)cc(-c3nc(OC(C)C)c4c(N5CC6CCC(C5)N6)nc(OC[C@@H]5C[C@H](F)CN5C)nc4c3F)c12] | 632.4 | 4.0 TFA salt, ¹H NMR (400 MHz, methanol-d₄, ppm): δ 7.85-7.77 (m, 1H), 7.36-7.25 (m, 2H), 7.23-7.17 (m, 1H), 5.61-5.38 (m, 2H), 5.03-4.91 (m, 1H), 4.73-4.40 (m, 3H), 4.27-4.16 (m, 2H), 4.14-3.82 (m, 4H), 3.57-3.42 (m, 1H), 3.38-3.32 (m, 1H), 3.18 (s, 3H), 2.96-2.72 (m, 1H), 2.46- 2.28 (m, 1H), 2.15-1.92 (m, 4H), 1.47-1.36 (m, 6H). ¹⁹F NMR (376 MHz, methanol-d₄, ppm): δ −112.3 (1F), −151.4 (1F), −173.7 (1F). |
| 208 | 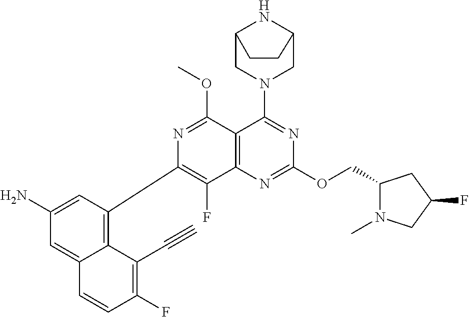 [SMILES: C#Cc1c(F)ccc2cc(N)cc(-c3nc(OC)c4c(N5CC6CCC(C5)N6)nc(OC[C@@H]5C[C@@H](F)CN5C)nc4c3F)c12] | 604.3 | 3.0 TFA salt, ¹H NMR (400 MHz, methanol-d₄, ppm): δ 7.85-7.77 (m, 1H), 7.34-7.23 (m, 3H), 5.57-5.38 (m, 1H), 5.03-4.92 (m, 1H), 4.76-4.71 (m, 1H), 4.52-4.36 (m, 2H), 4.32-4.17 (m, 3H), 4.16-3.95 (m, 4H), 3.81-3.54 (m, 3H), 3.39-3.34 (m, 1H), 3.18 (s, 3H), 2.74-2.59 (m, 1H), 2.52- 2.39 (m, 1H), 2.24-2.03 (m, 4H). ¹⁹F NMR (376 MHz, methanol-d₄, ppm): δ −112.26 (1F), −150.43 (1F), −174.16 (1F). |
| 209 | 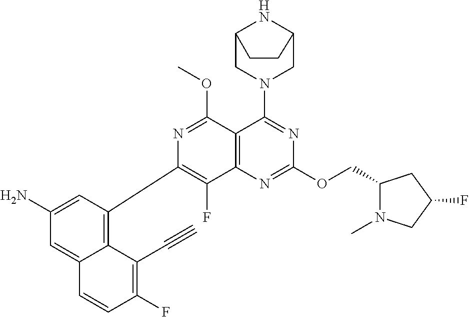 [SMILES: C#Cc1c(F)ccc2cc(N)cc(-c3nc(OC)c4c(N5CC6CCC(C5)N6)nc(OC[C@@H]5C[C@H](F)CN5C)nc4c3F)c12] | 604.4 | 4.0 TFA salt, ¹H NMR (400 MHz, methanol-d₄, ppm): δ 7.84-7.77 (m, 1H), 7.36-7.20 (m, 3H), 5.55-5.38 (m, 1H), 4.98-4.91 (m, 1H), 4.74-4.64 (m, 1H), 4.55-4.36 (m, 2H), 4.26-4.18 (m, 2H), 4.16-4.08 (m, 1H), 4.06 (s, 3H), 4.02- 3.87 (m, 1H), 3.80-3.67 (m, 2H), 3.58-3.42 (m, 1H), 3.36- 3.31 (m, 1H), 3.18 (s, 3H), 2.94-2.74 (m, 1H), 2.45-2.28 (m, 1H), 2.20-2.04 (m, 4H). ¹⁹F NMR (376 MHz, methanol-d₄, ppm): δ −112.3 (1F), −150.4 (1F), −173.6 (1F). |
| 210 | 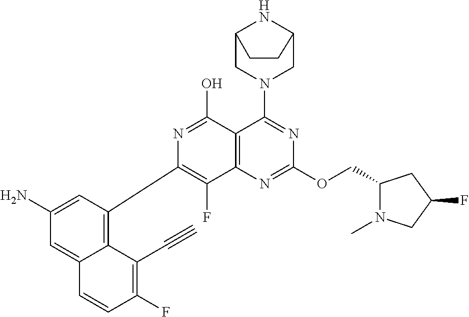 [SMILES: C#Cc1c(F)ccc2cc(N)cc(-c3nc(O)c4c(N5CC6CCC(C5)N6)nc(OC[C@@H]5C[C@@H](F)CN5C)nc4c3F)c12] | 590.4 | 4.0 TFA salt, ¹H NMR (400 MHz, methanol-d₄, ppm): δ 7.81-7.74 (m, 1H), 7.32-7.20 (m, 3H), 5.57-5.40 (m, 1H), 4.95-4.90 (m, 1H), 4.75-4.69 (m, 1H), 4.56-4.37 (m, 2H), 4.31-3.94 (m, 4H), 3.73-3.55 (m, 4H), 3.17 (s, 3H), 2.72- 2.62 (m, 1H), 2.52-2.24 (m, 3H), 2.17-2.03 (m, 2H). ¹⁹F NMR (376 MHz, methanol-d₄, ppm): δ −112.27 (1F), −162.21 (1F), −174.20 (1F). |
| 214 | 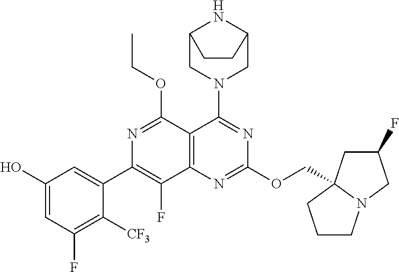 [SMILES: CCOc1nc(-c2cc(O)cc(F)c2C(F)(F)F)c(F)c2nc(OC[C@@]34CCCN3C[C@H](F)C4)nc(N3CC4CCC(C3)N4)c12] | 639.4 | 2.0 TFA salt, ¹H NMR (400 MHz, methanol-d₄, ppm): δ 6.80 (dd, J = 12.8, 1.6 Hz, 1H), 6.71-6.69 (m, 1H), 5.66-5.49 (m, 1H), 4.70-4.64 (m, 2H), 4.60-4.42 (m, 4H), 4.21-4.15 (m, 2H), 4.09-3.77 (m, 5H), 3.51-3.42 (m, 1H), 2.78-2.51 (m, 2H), 2.47-2.29 (m, 3H), 2.25-1.95 (m, 5H), 1.44 (t, J = 6.8 Hz, 3H). ¹⁹F NMR (376 MHz, methanol-d₄, ppm): δ −56.61 (3F), −112.28 (1F), −151.91 (1F), −174.09 (1F). |
| 215 | 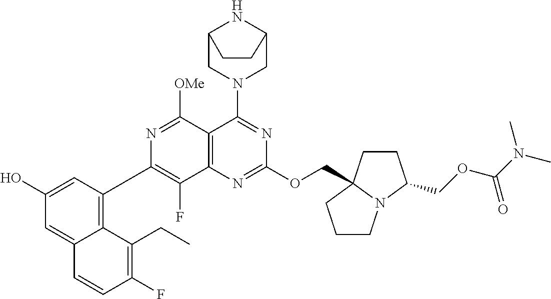 [SMILES: CCc1c(F)ccc2cc(O)cc(-c3nc(OC)c4c(N5CC6CCC(C5)N6)nc(OC[C@]56CCCN5[C@@H](COC(=O)N(C)C)CC6)nc4c3F)c12] | 718.5 | 2.0 TFA salt, ¹H NMR (400 MHz, methanol-d₄, ppm): δ 7.72-7.61 (m, 1H), 7.33-7.19 (m, 2H), 7.10-7.02 (m, 1H), 4.75-4.59 (m, 2H), 4.56-4.41 (m, 3H), 4.39-4.17 (m, 4H), 4.05 (s, 3H), 3.82-3.68 (m, 2H), 3.66-3.54 (m, 1H), 3.48- 3.38 (m, 1H), 2.99-2.79 (m, 6H), 2.62-2.29 (m, 4H), 2.28- 2.01 (m, 10H), 0.91-0.73 (m, 3H). ¹⁹F NMR (376 MHz, methanol-d₄, ppm): δ −121.38 (1F), −149.34 (1F). |
| 216 | 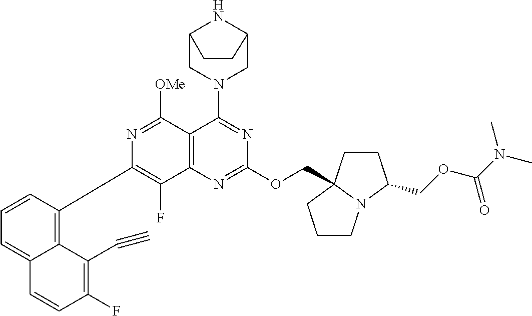 [SMILES: C#Cc1c(F)ccc2cccc(-c3nc(OC)c4c(N5CC6CCC(C5)N6)nc(OC[C@]56CCCN5[C@@H](COC(=O)N(C)C)CC6)nc4c3F)c12] | 698.5 | 3.0 TFA salt, ¹H NMR (400 MHz, methanol-d₄, ppm): δ 8.14-8.05 (m, 2H), 7.70-7.61 (m, 2H), 7.49-7.40 (m, 1H), 4.75-4.59 (m, 2H), 4.55-4.40 (m, 3H), 4.40-4.31 (m, 1H), 4.30-4.17 (m, 3H), 4.06 (s, 3H), 3.82-3.69 (m, 2H), 3.66- 3.54 (m, 1H), 3.50-3.37 (m, 2H), 2.94-2.84 (m, 6H), 2.47- 2.03 (m, 12H). ¹⁹F NMR (376 MHz, methanol-d₄, ppm): δ −106.9(1F), −150.3 (1F). |
| 221 | 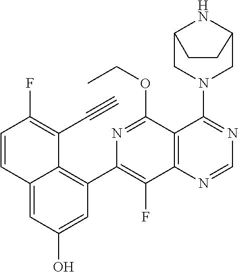 [SMILES: C#Cc1c(F)ccc2cc(O)cc(-c3nc(OCC)c4c(N5CC6CCC(C5)N6)ncnc4c3F)c12] | 488.2 | ¹H NMR (400 MHz, DMSO-d₆, ppm): δ 10.14 (s, 1H), 8.55 (s, 1H), 7.98 (dd, J = 9.2, 6.0 Hz, 1H), 7.47 (t, J = 9.0 Hz, 1H), 7.39 (d, J = 2.4 Hz, 1H), 7.23 (d, J = 2.4 Hz, 1H), 4.48- 4.28 (m, 2H), 4.26-3.98 (m, 2H), 3.81 (s, 1H), 3.55-3.41 (m, 4H), 1.61-1.46 (m, 4H), 1.34 (t, J = 7.0 Hz, 3H). ¹⁹F NMR (376 MHz, methanol-d₄, ppm): δ −110.44 (1F), −149.25 (1F). |
| 222 | 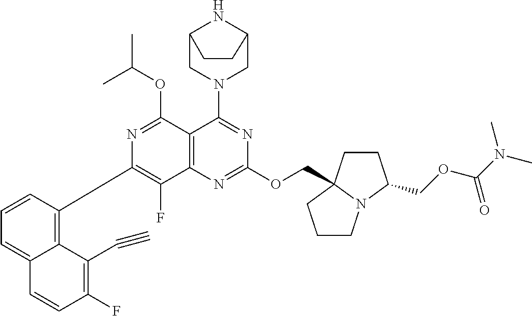 [SMILES: C#Cc1c(F)ccc2cccc(-c3nc(OC(C)C)c4c(N5CC6CCC(C5)N6)nc(OC[C@]56CCCN5[C@@H](COC(=O)N(C)C)CC6)nc4c3F)c12] | 726.6 | 3.0 TFA salt, ¹H NMR (400 MHz, methanol-d₄, ppm): δ 8.12-8.08 (m, 2H), 7.66-7.61 (m, 2H), 7.42 (t, J = 8.8 Hz, 1H), 5.56-5.49 (m, 1H), 4.73-4.47 (m, 5H), 4.38-4.19 (m, 4H), 3.92-3.83 (m, 2H), 3.62-3.58 (m, 1H), 3.46-3.40 (m, 2H), 2.91-2.85 (m, 6H), 2.43-2.36 (m, 2H), 2.26-1.99 (m, 10H), 1.42-1.39 (m, 6H). ¹⁹F NMR (376 MHz, methanol-d₄, ppm): δ −107.02 (1F), −151.18 (1F). |
| 223 | 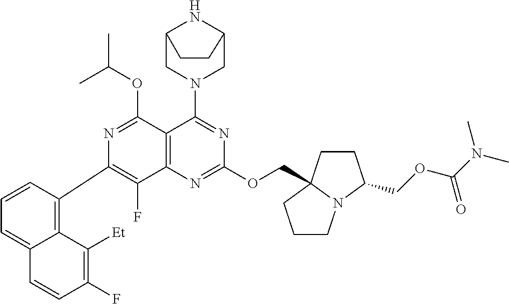 [SMILES: CCc1c(F)ccc2cccc(-c3nc(OC(C)C)c4c(N5CC6CCC(C5)N6)nc(OC[C@]56CCCN5[C@@H](COC(=O)N(C)C)CC6)nc4c3F)c12] | 730.5 | 3.0 TFA salt, ¹H NMR (400 MHz, methanol-d₄, ppm): δ 8.04-8.02 (m, 1H), 7.93-7.89 (m, 1H), 7.55-7.51 (m, 1H), 7.46-7.45 (m, 1H), 7.38-7.33 (m, 1H), 5.56-5.50 (m, 1H), 4.73-4.68 (m, 1H), 4.64-4.47 (m, 4H), 4.38-4.33 (m, 1H), 4.33-4.19 (m, 3H), 3.90-3.85 (m, 2H), 3.60-3.58 (m, 1H), 3.46-3.39 (m, 1H), 2.93-2.87 (m, 6H), 2.69-2.66 (m, 1H), 2.43-2.36 (m, 3H), 2.25-2.22 (m, 2H), 2.15-1.97 (m, 8H), 1.41-1.39 (m, 6H), 0.87-0.82 (m, 3H). ¹⁹F NMR (376 MHz, methanol-d₄, ppm): δ −116.74 (1F), −150.02 (1F). |
| 225 | 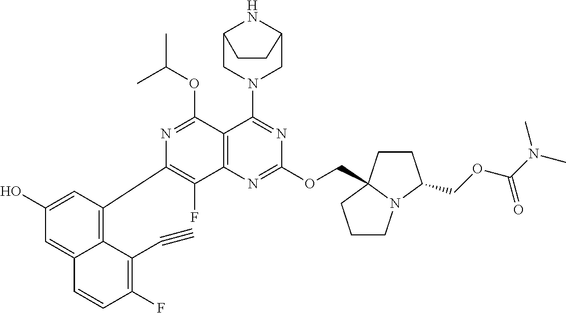 [SMILES: C#Cc1c(F)ccc2cc(O)cc(-c3nc(OC(C)C)c4c(N5CC6CCC(C5)N6)nc(OC[C@]56CCCN5[C@@H](COC(=O)N(C)C)CC6)nc4c3F)c12] | 742.5 | 3.0 TFA salt, ¹H NMR (400 MHz, methanol-d₄, ppm): δ 7.87-7.83 (m, 1H), 7.34-7.29 (m, 2H), 7.19-7.18 (m, 1H), 5.58-5.51 (m, 1H), 4.74-4.69 (m, 1H), 4.63-4.46 (m, 4H), 4.39-4.32 (m, 1H), 4.25-4.19 (m, 3H), 3.90-3.82 (m, 2H), 3.62-3.58 (m, 1H), 3.48-3.42 (m, 1H), 3.35-3.33 (m, 1H), 2.90-2.84 (m, 6H), 2.43-1.99 (m, 12H), 1.43-1.39 (m, 6H). ¹⁹F NMR (376 MHz, methanol-d₄, ppm): δ −111.82 (1F), −151.15(1F). |
| 226 | 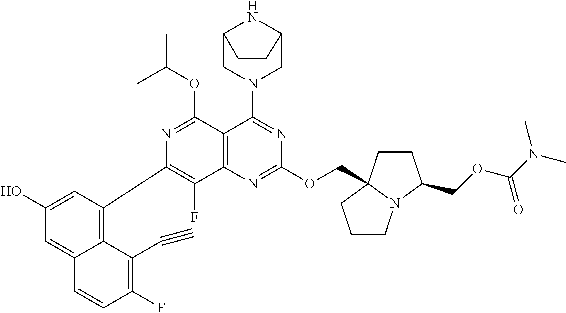 [SMILES: C#Cc1c(F)ccc2cc(O)cc(-c3nc(OC(C)C)c4c(N5CC6CCC(C5)N6)nc(OC[C@]56CCCN5[C@H](COC(=O)N(C)C)CC6)nc4c3F)c12] | 742.2 | 4.0 TFA salt, ¹H NMR (400 MHz, methanol-d₄, ppm): δ 7.86 (dd, J = 9.2, 5.6 Hz, 1H), 7.37-7.27 (m, 2H), 7.19 (dd, J = 7.2, 2.4 Hz, 1H), 5.62-5.49 (m, 1H), 4.72-4.65 (m, 2H), 4.61- 4.39 (m, 3H), 4.35-4.16 (m, 3H), 3.94-3.80 (m, 3H), 3.74- 3.61 (m, 1H), 3.58-3.49 (m, 1H), 3.42-3.34 (m, 1H), 2.91- 2.73 (m, 6H), 2.58-2.44 (m, 1H), 2.36-1.95 (m, 11H), 1.49- 1.36 (m, 6H). ¹⁹F NMR (376 MHz, methanol-d₄, ppm): −111.80 (1 F), −151.13(1 F). |
| 227 | 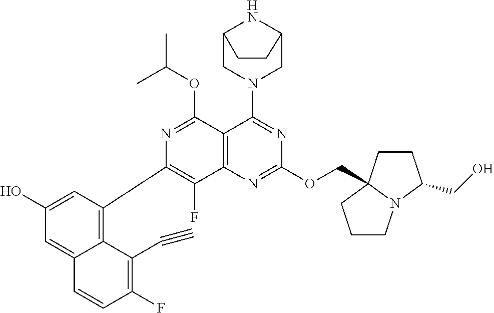 [SMILES: C#Cc1c(F)ccc2cc(O)cc(-c3nc(OC(C)C)c4c(N5CC6CCC(C5)N6)nc(OC[C@]56CCCN5[C@@H](CO)CC6)nc4c3F)c12] | 671.4 | 4.0 TFA salt, ¹H NMR (400 MHz, methanol-d₄, ppm): δ 7.89-7.83 (m, 1H), 7.39-7.29 (m, 2H), 7.18 (d, J = 2.4 Hz, 1H), 5.56-5.53 (m, 1H), 4.71-4.48 (m, 4H), 4.22-4.19 (m, 2H), 4.08-3.75 (m, 5H), 3.63-3.59 (m, 1H), 3.48-3.41 (m, 1H), 3. 38-3.32 (m, 1H), 2.55-2.30 (m, 2H), 2.28-1.99 (m, 10H), 1.42-1.40 (m, 6H). ¹⁹F NMR (376 MHz, methanol-d₄, ppm): δ −111.87 (1F), −151.13 (1F). |
| 228 | 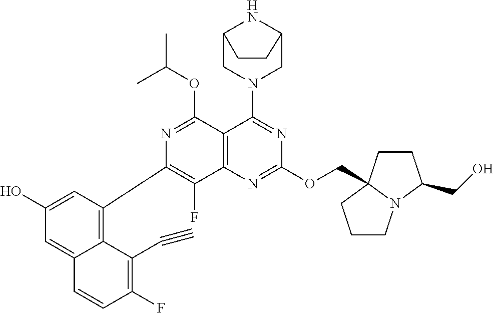 [SMILES: C#Cc1c(F)ccc2cc(O)cc(-c3nc(OC(C)C)c4c(N5CC6CCC(C5)N6)nc(OC[C@]56CCCN5[C@H](CO)CC6)nc4c3F)c12] | 671.4 | 4.0 TFA salt, ¹H NMR (400 MHz, methanol-d₄, ppm): δ 7.89-7.82 (m, 1H), 7.37-7.27 (m, 2H), 7.19 (d, J = 2.0 Hz, 1H), 5.61-5.45 (m, 1H), 4.71-4.41 (m, 4H), 4.26-4.10 (m, 2H), 3.95-3.72 (m, 4H), 3.67-3.57 (m, 2H), 3.57-3.48 (m, 1H), 3.41-3.34 (m, 1H), 2.53-2.43 (m, 1H), 2.29-1.97 (m, 11H), 1.46-1.37 (m, 6H). ¹⁹F NMR (376 MHz, methanol-d₄, ppm): −111.87 (1F), −151.22 (1F). |
| 229 | 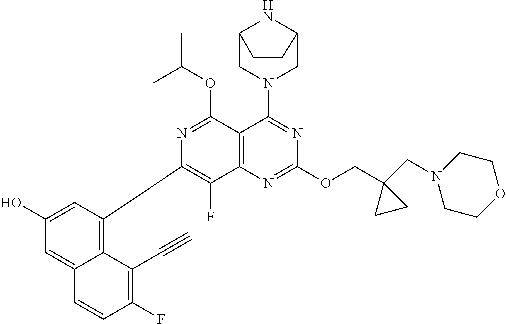 [SMILES: C#Cc1c(F)ccc2cc(O)cc(-c3nc(OC(C)C)c4c(N5CC6CCC(C5)N6)nc(OCC5(CN6CCOCC6)CC5)nc4c3F)c12] | 671.5 | 3.0 TFA salt, ¹H NMR (400 MHz, methanol-d₄, ppm): δ 7.90-7.80 (m, 1H), 7.36-7.28 (m, 2H), 7.22-7.16 (m, 1H), 5.60-5.49 (m, 1H), 4.57-4.42 (m, 4H), 4.25-4.15 (m, 2H), 4.09-3.67 (m, 8H), 3.44-3.37 (m, 2H), 3.35-3.32 (m, 1H), 3.25-3.06 (m, 2H), 2.10-1.99 (m, 4H), 1.43-1.38 (m, 6H), 1.01-0.84 (m, 4H). ¹⁹F NMR (376 MHz, methanol-d₄, ppm): δ −111.8 (1F), −150.9 (1F). |
| 233 | 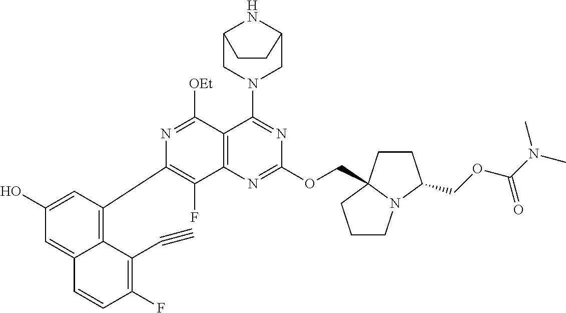 [SMILES: C#Cc1c(F)ccc2cc(O)cc(-c3nc(OCC)c4c(N5CC6CCC(C5)N6)nc(OC[C@]56CCCN5[C@@H](COC(=O)N(C)C)CC6)nc4c3F)c12] | 728.5 | 3.0 TFA salt, ¹H NMR (400 MHz, methanol-d₄, ppm): δ 7.87-7.83 (m, 1H), 7.34-7.29 (m, 2H), 7.24-7.16 (m, 1H), 4.75-4.45 (m, 7H), 4.39-4.14 (m, 4H), 3.89-3.73 (m, 2H), 3.65-3.55 (m, 1H), 3.49-3.39 (m, 1H), 3.37-3.33 (m, 1H), 3.0-2.78 (m, 6H), 2.48-2.30 (m, 2H), 2.28-2.18 (m, 2H), 2.17-2.01 (m, 8H), 1.43 (t, J = 7.2 Hz, 3H). ¹⁹F NMR (376 MHz, methanol-d₄, ppm): δ −111.82 (1F), −150.71 (1F). |
| 235 | 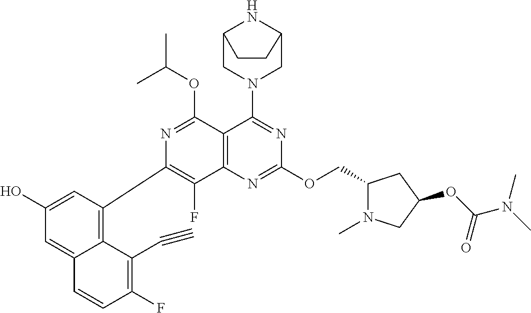 [SMILES: C#Cc1c(F)ccc2cc(O)cc(-c3nc(OC(C)C)c4c(N5CC6CCC(C5)N6)nc(OC[C@@H]5C[C@@H](OC(=O)N(C)C)CN5C)nc4c3F)c12] | 702.0 | 3.0 TFA salt, ¹H NMR (400 MHz, methanol-d₄, ppm): δ 7.87-7.84 (m, 1H), 7.34-7.30 (m, 2H), 7.19 (d, J = 2.4 Hz, 1H), 5.58-5.52 (m, 1H), 5.34-5.28 (m, 1H), 4.98-4.93 (m, 1H), 4.78-4.69 (m, 1H), 4.55-4.52 (m, 2H), 4.22-4.19 (m, 3H), 4.10-4.00 (m, 1H), 3.88-3.84 (m, 2H), 3.55-3.45 (m, 1H), 3.42-3.36 (m, 1H), 3.16 (s, 3H), 2.97-2.92 (m, 6H), 2.55-2.42 (m, 2H), 2.09-2.00 (m, 4H), 1.43-1.40 (m, 6H). ¹⁹F NMR (376 MHz, methanol-d₄, ppm): δ −111.83 (1F), −151.42 (1F). |
| 236 | 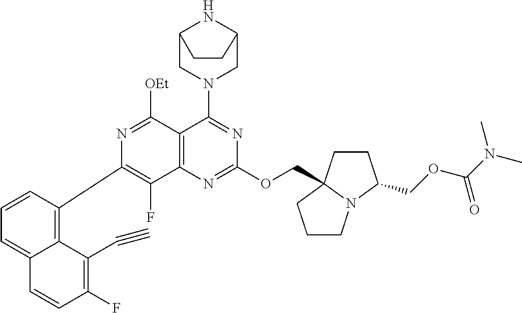 [SMILES: C#Cc1c(F)ccc2cccc(-c3nc(OCC)c4c(N5CC6CCC(C5)N6)nc(OC[C@]56CCCN5[C@@H](COC(=O)N(C)C)CC6)nc4c3F)c12] | 712.4 | 3.0 TFA salt, ¹H NMR (400 MHz, methanol-d₄, ppm): δ 8.16-8.05 (m, 2H), 7.69-7.60 (m, 2H), 7.48-7.40 (m, 1H), 4.75-4.68 (m, 1H), 4.67-4.42 (m, 6H), 4.40-4.31 (m, 1H), 4.30-4.15 (m, 3H), 3.89-3.75 (m, 2H), 3.65-3.54 (m, 1H), 3.50-3.37 (m, 2H), 2.95-2.81 (m, 6H), 2.47-2.30 (m, 2H), 2.28-2.00 (m, 10H), 1.47-1.38 (m, 3H). ¹⁹F NMR (376 MHz, methanol-d₄, ppm): δ −107.0 (1F), −150.7 (1F). |
| 237 | 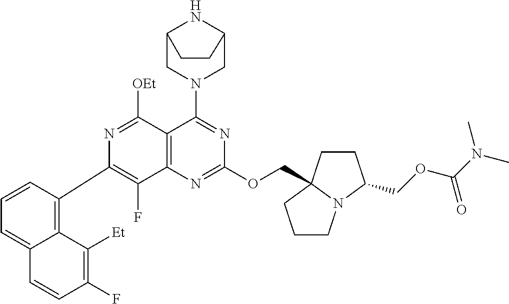 [SMILES: CCOc1nc(-c2cccc3ccc(F)c(CC)c23)c(F)c2nc(OC[C@]34CCCN3[C@@H](COC(=O)N(C)C)CC4)nc(N3CC4CCC(C3)N4)c12] | 716.5 | 3.0 TFA salt, ¹H NMR (400 MHz, methanol-d₄, ppm): δ 8.07-7.99 (m, 1H), 7.95-7.87 (m, 1H), 7.57-7.49 (m, 1H), 7.48-7.42 (m, 1H), 7.40-7.31 (m, 1H), 4.74-4.46 (m, 7H), 4.40-4.18 (m, 4H), 3.90-3.78 (m, 2H), 3.66-3.56 (m, 1H), 3.49-3.38 (m, 1H), 2.99-2.85 (m, 6H), 2.70-2.58 (m, 1H), 2.45-2.32 (m, 3H), 2.29-1.98 (m, 10H), 1.47-1.39 (m, 3H), 0.88-0.80 (m, 3H). ¹⁹F NMR (376 MHz, methanol-d₄, ppm): δ −111.67 (1F), −149.70 (1F). |
| 238 | 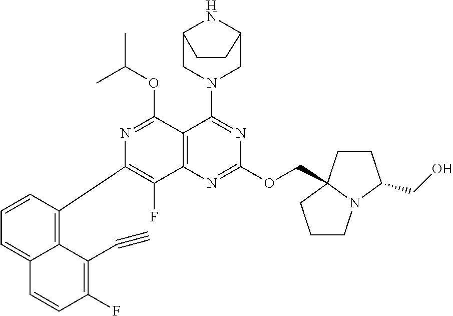 [SMILES: C#Cc1c(F)ccc2cccc(-c3nc(OC(C)C)c4c(N5CC6CCC(C5)N6)nc(OC[C@]56CCCN5[C@@H](CO)CC6)nc4c3F)c12] | 655.4 | 3.0 TFA salt, ¹H NMR (400 MHz, methanol-d₄, ppm): δ 8.18-8.04 (m, 2H), 7.68-7.58 (m, 2H), 7.50-7.36 (m, 1H), 5.60-5.45 (m, 1H), 4.69-4.41 (m, 4H), 4.25-4.16 (m, 2H), 4.11-3.95 (m, 1H), 3.94-3.83 (m, 3H), 3.82-3.74 (m, 1H), 3.65-3.57 (m, 1H), 3.51-3.37 (m, 2H), 2.45-2.31 (m, 2H), 2.26-2.18 (m, 1H), 2.15-1.95 (m, 9H), 1.41 (d, J = 6.4 Hz, 6H). ¹⁹F NMR (376 MHz, methanol-d₄, ppm): δ −107.09 (1F), −151.23 (1F). |
| 239 | 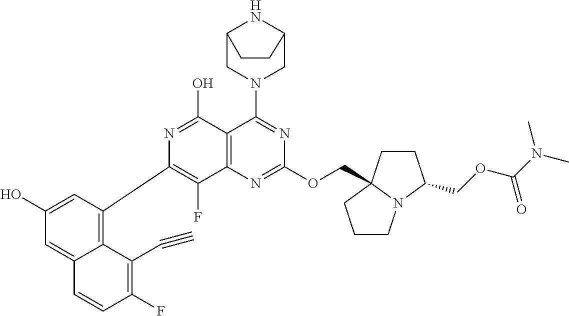 [SMILES: C#Cc1c(F)ccc2cc(O)cc(-c3nc(O)c4c(N5CC6CCC(C5)N6)nc(OC[C@]56CCCN5[C@@H](COC(=O)N(C)C)CC6)nc4c3F)c12] | 700.5 | 3.0 TFA salt, ¹H NMR (400 MHz, methanol-d₄, ppm): δ 7.90-7.87 (m, 1H), 7.40-7.35 (m, 2H), 7.28-7.27 (m, 1H), 4.72-4.67 (m, 1H), 4.61-4.32 (m, 5H), 4.25-4.18 (m, 3H), 3.69-3.57 (m, 4H), 3.48-3.43 (m, 1H), 2.93-2.89 (m, 6H), 2.42-2.22 (m, 6H), 2.15-2.09 (m, 6H). ¹⁹F NMR (376 MHz, methanol-d₄, ppm): δ −110.71 (1F), −162.08 (1F). |
| 241 | 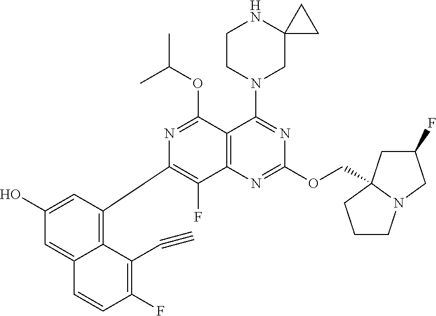 [SMILES: C#Cc1c(F)ccc2cc(O)cc(-c3nc(OC(C)C)c4c(N5CCNC6(CC6)C5)nc(OC[C@@]56CCCN5C[C@H](F)C6)nc4c3F)c12] | 659.4 | 4.0 TFA salt, ¹H NMR (400 MHz, methanol-d₄, ppm): δ7.85 (dd, J = 9.2, 5.6 Hz, 1H), 7.34-7.29 (m, 2H), 7.19 (d, J = 2.4 Hz, 1H), 5.62-5.49 (m, 2H), 4.73-4.64 (m, 2H), 4.18-3.82 (m, 7H), 3.60-3.58 (m, 2H), 3.49-3.43 (m,1H), 3.38 (d, J = 3.2 Hz, 1H), 2.69-2.14 (m, 6H), 1.43-1.40 (m, 6H), 1.17-1.05 (m, 4H). ¹⁹F NMR (376 MHz, methanol-d₄, ppm): δ −111.84 (1F), −151.22 (1F), −174.27 (1F). |
| 243 | 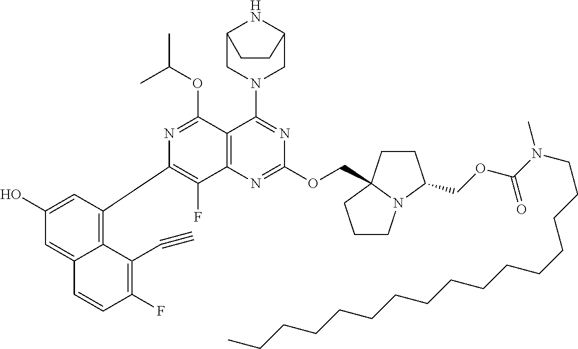 [SMILES: C#Cc1c(F)ccc2cc(O)cc(-c3nc(OC(C)C)c4c(N5CC6CCC(C5)N6)nc(OC[C@]56CCCN5[C@@H](COC(=O)N(C)CCCCCCCCCCCCCCCC)CC6)nc4c3F)c12] | [M + 2H] ²⁺/2 = 462.9 | ¹H NMR (400 MHz, methanol-d₄, ppm): δ 7.87-7.84 (m, 1H), 7.34-7.30 (m, 2H), 7.22-7.21 (m, 1H), 4.74-4.70 (m, 1H), 4.64-4.60 (m, 1H), 4.51-4.37 (m, 4H), 4.24-4.21 (m, 3H), 4.06 (d, J = 1.2 Hz, 3H), 3.75-3.70 (m, 2H), 3.63-3.55 (m, 1H), 3.48-3.42 (m, 1H), 3.35-3.33 (m, 1H), 3.25-3.15 (m, 2H), 2.88-2.82 (m, 3H), 2.43-2.30 (m, 2H), 2.27-2.20 (m, 2H), 2.17-2.05 (m, 8H), 1.54-1.45 (m, 2H), 1.30-1.20 (m, 26H), 0.90-0.86 (m, 3H). ¹⁹F NMR (376 MHz, methanol- d₄, ppm): δ −111.73 (1F), −150.26 (1F). |
| 245 | 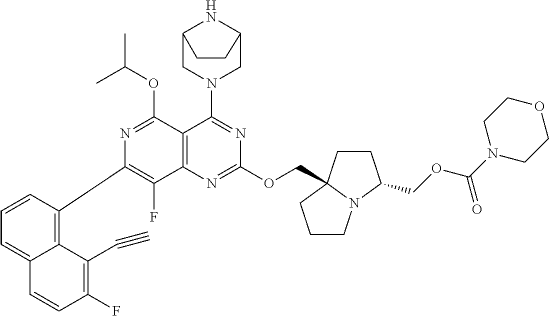 [SMILES: C#Cc1c(F)ccc2cccc(-c3nc(OC(C)C)c4c(N5CC6CCC(C5)N6)nc(OC[C@]56CCCN5[C@@H](COC(=O)N5CCOCC5)CC6)nc4c3F)c12] | 768.6 | 3.0 TFA salt, ¹H NMR (400 MHz, methanol-d₄, ppm): δ 8.15-8.11 (m, 2H), 7.72-7.60 (m, 2H), 7.52-7.42 (m, 1H), 5.62-5.48 (m, 1H), 4.73-4.64 (m, 2H), 4.59-4.49 (m, 3H), 4.42-4.35 (m, 1H), 4.35-4.21 (m, 3H), 3.93-3.85 (m, 2H), 3.70-3.58 (m, 5H), 3.56-3.38 (m, 6H), 2.50-2.34 (m, 2H), 2.34-2.22 (m, 2H), 2.20-1.98 (m, 8H), 1.50-1.37 (m, 6H). ¹⁹F NMR (376 MHz, methanol-d₄, ppm): δ −107.01(1F), −151.21(1F). |
| 247 | 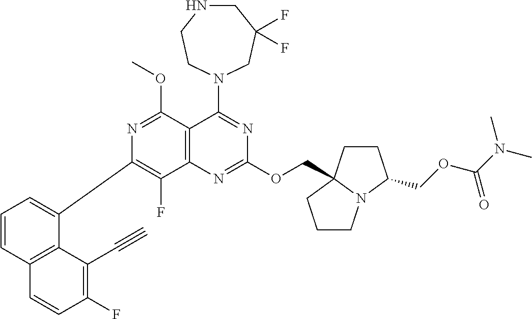 [SMILES: C#Cc1c(F)ccc2cccc(-c3nc(OC)c4c(N5CCNCC(F)(F)C5)nc(OC[C@]56CCCN5[C@@H](COC(=O)N(C)C)CC6)nc4c3F)c12] | 722.6 | 2.0 TFA salt, ¹H NMR (400 MHz, methanol-d₄, ppm): δ 8.14- 8.09 (m, 2H), 7.71-7.63 (m, 2H), 7.45 (t, J = 8.8 Hz, 1H), 4.74 (d, J = 12.0 Hz, 1H), 4.65 (dd, J = 12.0, 4.0 Hz, 1H), 4.59-4.32 (m, 4H), 4.30-4.15 (m, 3H), 4.07 (d, J = 0.8 Hz, 3H), 3.96-3.87 (m, 2H), 3.84-3.72 (m, 2H), 3.64-3.57 (m, 1H), 3.48-3.38 (m, 2H), 2.93-2.88 (m, 6H), 2.46-2.32 (m, 2H), 2.28-2.20 (m, 2H), 2.19-2.02 (m, 4H). ¹⁹F NMR (376 MHz, methanol-d₄, ppm): δ −96.83(2F), −106.87 (1F), −150.22 (1F). |

### Biological Example 1. Cell Assay

Ba/F3_KRAS^{G12D} cells (KYinno, China) were generated by transducing Ba/F3 parental cells with the recombinant KRAS^{G12D} lentivirus and followed by 1 ug/mL of puromycin selection and IL3 depletion. Cells were grown in RPMI 1640 medium supplemented with 10% fetal bovine serum, 100 U/mL penicillin and 100 μg/mL streptomycin at 37° C. in an atmosphere of 500 CO₂ in air. Cells were seeded at a density of 5×10³ per well into 96-well plate and incubated overnight. Serial diluted compounds were added to each well. Cells were were treated with the compounds for 3 days, after which cell-titer Glo reagent (Promega #G7572) was used to assess cell proliferation. The luminescence signal was then collected on Tecan Spark plate reader. Inhibition rate is calculated with the formula of % inhibition=100*(Control−well)/(Control−Blank). Cell growth inhibition of IC₅₀ is calculated with the equation of Y=Bottom+(Top−Bottom)/(1+10\hat{ }((LogIC₅₀−X)*HillSlope))

In Table 2 below, the IC₅₀ levels are described as I, II, or III, wherein I represents IC₅₀ value less than or equal to 500 nM; II represents IC₅₀ value between 500 nM to 5000 nM; and III represents IC₅₀ value more than 5000 nM.

TABLE 2
Inhibition of Ba/F3 KRAS^{G12D} Cell Proliferation
by Representative Compounds

| | BaF3_KRAS^{G12D} |
|---|---|
| Compound | IC₅₀ (nM) |
| 1 | II |
| 2 | III |
| 3 | III |
| 4 | III |
| 5 | III |
| 6 | I |
| 7 | III |
| 8 | II |
| 9 | III |
| 10 | III |
| 11 | III |
| 12 | II |
| 13 | II |
| 14 | II |
| 15 | III |
| 16 | II |
| 17 | III |
| 18 | II |
| 19 | III |
| 20 | II |
| 21 | II |
| 22 | III |
| 23 | II |
| 24 | II |
| 25 | II |
| 26 | III |
| 27 | II |
| 28 | II |
| 29 | II |
| 30 | I |
| 31 | II |
| 32 | I |
| 33 | III |
| 34 | III |
| 35 | III |
| 36 | I |
| 37 | III |
| 38 | III |
| 39 | III |
| 40 | III |
| 41 | III |
| 42 | II |
| 43 | II |
| 44 | III |
| 45 | III |
| 46 | II |
| 47 | III |
| 48 | II |
| 49 | II |
| 50 | II |
| 51 | III |
| 52 | II |
| 53 | I |
| 54 | II |
| 55 | III |
| 56 | II |
| 57 | III |
| 58 | II |
| 59 | III |
| 60 | III |
| 61 | II |
| 62 | II |
| 63 | II |
| 64 | III |
| 65 | III |
| 66 | II |
| 67 | II |
| 68 | III |
| 69 | II |
| 70 | I |
| 71 | III |
| 72 | II |
| 73 | III |
| 74 | III |
| 75 | III |
| 76 | I |
| 77 | III |
| 78 | II |
| 79 | III |
| 80 | II |
| 81 | I |
| 82 | I |
| 83 | II |
| 84 | II |
| 85 | I |
| 86 | II |
| 87 | III |
| 88 | III |
| 89 | I |
| 90 | II |
| 91 | II |
| 92 | III |
| 93 | I |
| 94 | II |
| 95 | I |
| 96 | III |
| 97 | III |
| 98 | II |
| 99 | II |
| 100 | III |
| 101 | II |
| 102 | III |
| 103 | I |
| 104 | III |
| 105 | III |
| 106 | I |
| 107 | II |
| 108 | III |
| 109 | III |
| 110 | II |
| 111 | III |
| 112 | I |
| 113 | I |
| 114 | I |
| 115 | III |
| 116 | II |
| 117 | I |
| 118 | I |
| 119 | III |
| 120 | I |
| 121 | II |
| 122 | II |
| 123 | II |
| 124 | I |
| 125 | I |
| 126 | I |
| 127 | I |
| 128 | I |
| 129 | I |
| 130 | I |
| 131 | I |
| 132 | I |
| 133 | II |
| 134 | I |
| 135 | I |
| 136 | I |
| 137 | I |
| 138 | I |
| 139 | I |
| 140 | I |
| 141 | I |
| 142 | I |
| 143 | I |
| 144 | I |
| 145 | II |
| 146 | I |
| 147 | I |
| 148 | I |
| 149 | II |
| 150 | I |
| 151 | III |
| 152 | II |
| 153 | II |
| 154 | II |
| 155 | I |
| 156 | I |
| 157 | I |
| 158 | I |
| 159 | I |
| 160 | I |
| 161 | I |
| 162 | I |
| 163 | I |
| 164 | I |
| 165 | I |
| 166 | I |
| 167 | I |
| 168 | I |
| 169 | I |
| 170 | I |
| 171 | I |
| 172 | I |
| 173 | I |
| 174 | II |
| 175 | I |
| 176 | I |
| 177 | I |
| 178 | I |
| 179 | I |
| 180 | I |
| 181 | I |
| 182 | I |
| 183 | I |
| 184 | I |
| 185 | I |
| 186 | II |
| 187 | II |
| 188 | II |
| 189 | II |
| 190 | I |
| 191 | I |
| 192 | I |
| 193 | I |
| 194 | I |
| 195 | I |
| 196 | II |
| 197 | I |
| 198 | I |
| 199 | I |
| 200 | I |
| 201 | II |
| 202 | I |
| 203 | I |
| 204 | I |
| 205 | I |
| 206 | I |
| 207 | I |
| 208 | I |
| 209 | I |
| 210 | II |
| 211 | II |
| 212 | I |
| 213 | I |
| 214 | II |
| 215 | I |
| 216 | I |
| 217 | I |
| 218 | I |
| 219 | I |
| 220 | I |
| 221 | I |
| 222 | I |
| 223 | I |
| 224 | I |
| 225 | I |
| 226 | I |
| 227 | I |
| 228 | I |
| 229 | I |
| 230 | I |
| 231 | I |
| 232 | I |
| 233 | I |
| 234 | I |
| 235 | I |
| 236 | I |
| 237 | I |
| 238 | I |
| 239 | II |
| 240 | I |
| 241 | I |
| 242 | I |
| 243 | I |
| 244 | I |
| 245 | I |
| 246 | II |
| 247 | II |

### Biological Example 2. KRAS^{G12D} Protein Binding Assay

The Temperature-dependent Fluorescence (TdF) assay was used to analyze binding affinity of compound to recombinant human KRAS^{G12D} protein. The TdF assay was conducted in the 96-well-based real-time fluorescence plate reader (ABI 7500 or Roche LightCycler 480). Fluorescent dye Sypro Orange (Sigma) was used to monitor the protein folding-unfolding transition. Protein-compound binding was gauged by the shift in the unfolding transition temperature (ΔTm) acquired with and without compound. Each reaction sample consists of 6 μM KRAS^{G12D} Protein, 10 μM compound, and Sypro Orange dye (in 1% DMSO) in 20 μL reaction buffer (25 mM HEPES pH 7.5, 150 mM NaCl, 10 mM MgCl₂). The sample plate was heated from 30° C. to 95° C. with a thermal ramping rate of 0.5%, taking a fluorescence reading every 0.4° C. using a CY3 channel matching the excitation and emission wavelengths of Sypro Orange (λ ex 470 nm; λ em 570 nm). Binding affinity (K_{d} value) was calculated based on the degree of fluorescent shift of the protein with and without compound.

In Table 3 below, the K_{d} levels are described as I, II, or III, wherein I represents K_{d} value less than or equal to 500 nM; II represents K_{d} value in the range of 500 nM to 5000 nM; and III represents K_{d} value more than 5000 nM.

TABLE 3
Binding Affinity of Representative Compounds

| | TdF |
|---|---|
| Compound | K_{d} (nM) |
| 1 | III |
| 2 | III |
| 3 | III |
| 4 | III |
| 5 | III |
| 6 | I |
| 7 | III |
| 8 | III |
| 9 | III |
| 10 | III |
| 11 | III |
| 12 | I |
| 13 | III |
| 14 | III |
| 15 | III |
| 16 | III |
| 17 | III |
| 18 | III |
| 19 | III |
| 20 | III |
| 21 | III |
| 22 | III |
| 23 | III |
| 24 | III |
| 25 | III |
| 26 | III |
| 27 | III |
| 28 | III |
| 29 | III |
| 30 | I |
| 31 | III |
| 32 | I |
| 33 | III |
| 34 | III |
| 35 | III |
| 36 | I |
| 37 | III |
| 38 | III |
| 39 | III |
| 40 | III |
| 41 | III |
| 42 | III |
| 43 | III |
| 44 | III |
| 45 | III |
| 46 | III |
| 47 | I |
| 48 | III |
| 49 | III |
| 50 | III |
| 51 | III |
| 52 | III |
| 53 | III |
| 54 | III |
| 55 | III |
| 56 | III |
| 57 | III |
| 58 | II |
| 59 | III |
| 60 | III |
| 61 | I |
| 62 | III |
| 63 | III |
| 64 | III |
| 65 | II |
| 66 | III |
| 67 | III |
| 68 | I |
| 69 | III |
| 70 | I |
| 71 | II |
| 72 | I |
| 73 | I |
| 74 | I |
| 75 | I |
| 76 | I |
| 77 | III |
| 78 | I |
| 79 | III |
| 80 | II |
| 81 | I |
| 82 | III |
| 83 | III |
| 84 | II |
| 85 | I |
| 86 | III |
| 87 | II |
| 88 | III |
| 89 | I |
| 90 | II |
| 91 | II |
| 92 | II |
| 93 | I |
| 94 | I |
| 95 | I |
| 96 | I |
| 97 | III |
| 98 | III |
| 99 | III |
| 100 | III |
| 101 | III |
| 102 | III |
| 103 | I |
| 104 | III |
| 105 | II |
| 106 | I |
| 107 | III |
| 108 | III |
| 109 | III |
| 110 | III |
| 111 | III |
| 112 | I |
| 113 | I |
| 114 | I |
| 115 | I |
| 116 | III |
| 117 | I |
| 118 | I |
| 119 | II |
| 120 | I |
| 121 | I |
| 122 | III |
| 123 | III |
| 124 | I |
| 125 | I |
| 126 | I |
| 127 | I |
| 128 | I |
| 129 | I |
| 130 | I |
| 131 | I |
| 132 | I |
| 133 | I |
| 134 | I |
| 135 | I |
| 136 | I |
| 137 | I |
| 138 | I |
| 139 | I |
| 140 | I |
| 141 | I |
| 147 | I |
| 148 | I |
| 159 | I |
| 160 | I |
| 163 | I |
| 165 | I |
| 166 | I |
| 171 | I |
| 173 | I |
| 176 | I |
| 195 | I |
| 197 | I |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or Formula A, or a pharmaceutically acceptable salt thereof: 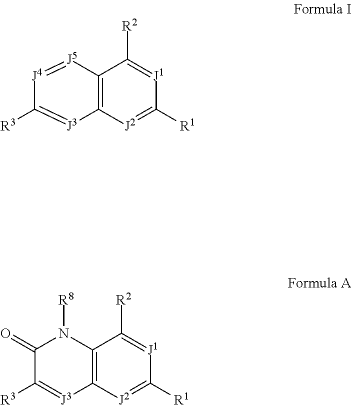
wherein:
J¹ is CR⁹ or N;
J² is CR¹⁰ or N;
J³ is CR¹¹ or N;
J⁴ is CR¹² or N;
J⁵ is CR^{12A} or N;
provided that in Formula I, at least one of J¹ and J² is N, and when both J¹ and J² are N, then at least one of J³, J⁴ and J⁵ is N;
R¹ is hydrogen, -(L¹)ₘ₁-OR²⁰, halogen, -(L¹)ₘ₁-NR³⁰R³¹, or an optionally substituted heterocyclic or heteroaryl ring;
R² is a ring or ring-chain structure, which has a basic functional group with a pKa of the conjugated acid of about 5 or higher, or an acylated derivative thereof (i.e., the basic functional group, such as a basic NH, is bonded with an acyl group);
R³ is an optionally substituted aryl or an optionally substituted heteroaryl,
R⁸ is hydrogen, optionally substituted C₁₋₆ alkyl (e.g., methyl), or optionally substituted C₃₋₁₀ cycloalkyl,
R⁹ and R¹⁰ at each occurrence is independently hydrogen, halogen, cyano, optionally substituted C₁₋₄ alkyl (e.g., methyl, ethyl, CF₃, etc.), optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹, R¹² and R^{12A} at each occurrence is independently hydrogen, F, Cl, Br, I, CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, or cyclobutoxy; and
wherein:
m1 is 0 or 1, and when m1 is 1, L¹ is an optionally substituted alkylene, an optionally substituted carbocyclylene, an optionally substituted heterocyclylene;
R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring or one of R³⁰ and R³¹ together with a suitable atom of L¹ and any intervening atoms form an optionally substituted heterocyclic or heteroaryl ring.

2. The compound of claim 1, or a pharmaceutically acceptable salt thereof, characterized as having one of the following formulae:

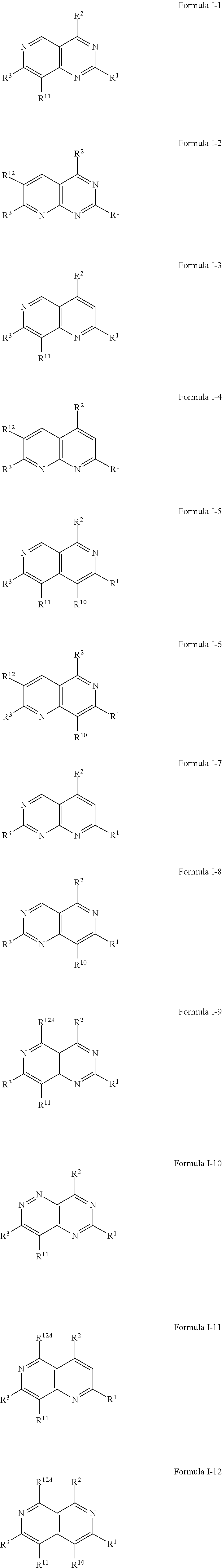

3. The compound of claim 1 or 2, or a pharmaceutically acceptable salt thereof, wherein when present, R¹⁰ in Formula I or A is hydrogen, halogen (e.g., Cl), C₁₋₄ alkyl optionally substituted with 1-3 F, e.g., methyl, ethyl, CF₃, etc., cyclopropyl, cyclobutyl, 5- or 6-membered heteroaryl having 1-4 heteroatoms independently selected from N, O, and S, such as pyrrazolyl, oxazolyl, pyridyl, pyrimidinyl, pyridazinyl, pyrazinyl, etc., wherein the heteroaryl is optionally substituted with 1-3 substituents independently selected from halogen, CN, C₁₋₄ alkyl optionally substituted with 1-3 F, e.g., methyl, ethyl, CF₃, etc., C₃₋₆ cycloalkyl (e.g., cyclopropyl, cyclobutyl) optionally substituted with one or more substituents independently selected from methyl, F, OH, and methoxy, and C₁₋₄ alkoxy optionally substituted with 1-3 F, e.g., methoxy, ethoxy, —OCF₃, etc.

4. The compound of claim 1 or 2, or a pharmaceutically acceptable salt thereof, wherein when present, R¹⁰ in Formula I or A is hydrogen, F, Cl, methyl, ethyl, isopropyl, CF₃, cyclopropyl, or cyclobutyl.

5. The compound of claim 1 or 2, or a pharmaceutically acceptable salt thereof, wherein when present, R¹⁰ in Formula I or A is 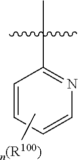
wherein R¹⁰⁰ at each occurrence is independently halogen, CN, C₁₋₄ alkyl optionally substituted with 1-3 F, e.g., methyl, ethyl, CF₃, etc., C₃₋₆ cycloalkyl (e.g., cyclopropyl, cyclobutyl) optionally substituted with one or more substituents independently selected from methyl, F, OH, and methoxy, and C₁₋₄ alkoxy optionally substituted with 1-3 F, e.g., methoxy, ethoxy, —OCF₃, etc.; and n is 0, 1, 2, or 3, preferably, n is 0, 1, or 2.

6. The compound of any one of claims 1-5, or a pharmaceutically acceptable salt thereof, wherein when present, R¹¹ in Formula I or A is hydrogen, F, Cl, —CN, —OH, methoxy, ethoxy, —O—CH₂-cyclopropyl, —C(O)NHMe, CF₃, methyl, ethyl, isopropyl, or cyclopropyl.

7. The compound of any one of claims 1-5, or a pharmaceutically acceptable salt thereof, wherein when present, R¹¹ in Formula I or A is hydrogen, F or Cl, or R¹¹ in Formula I or A is methyl.

8. The compound of any one of claims 1-7, or a pharmaceutically acceptable salt thereof, wherein when present, R¹² in Formula I is hydrogen, F, Cl, —CN, —OH, methoxy, ethoxy, —O—CH₂-cyclopropyl, —C(O)NHMe, CF₃, methyl, ethyl, isopropyl, or cyclopropyl, and wherein when present, R^{12A} in Formula I is hydrogen, F, Cl, —CN, —OH, methoxy, ethoxy, —O—CH₂-cyclopropyl, —C(O)NHMe, CF₃, methyl, ethyl, isopropyl, or cyclopropyl, preferably, when present, R^{12A} in Formula I is hydrogen, methyl, Cl, or methoxy or R^{12A} in Formula I is Cl, —OH, methoxy, difluoromethoxy, ethoxy, isopropoxy, —O—CH₂-cyclopropyl, —O—CH₂—CH₂-cyclopropyl, —C(O)NHMe, —O—CH₂—C(O)NHMe, —O—CH₂—CF₃, —O—CH₂—CHF₂, methyl, CHF₂, CF₃, ethyl, isopropyl, or cyclopropyl.

9. The compound of any one of claims 1-7, or a pharmaceutically acceptable salt thereof, wherein when present, R¹² in Formula I is hydrogen, F or Cl, and wherein when present, R^{12A} in Formula I is hydrogen or C₁₋₄ alkyl optionally substituted with F, such as methyl, or R^{12A} in Formula I is Cl or methoxy, or in the case of Formula A, R⁸ is hydrogen or C₁₋₄ alkyl optionally substituted with F, such as methyl.

10. The compound of any one of claims 1-9, or a pharmaceutically acceptable salt thereof, wherein R¹ in Formula I or A is —OR²⁰, wherein R²⁰ is a —C₁₋₆ alkylene-R¹⁰¹, wherein R¹⁰¹ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring;
R³² and R³³ are independently hydrogen, a nitrogen protecting group, an optionally substituted C₁₋₆ alkyl, an optionally substituted carbocyclic ring, or an optionally substituted heterocyclic ring; or R³² and R³³ are joined to form an optionally substituted heterocyclic or heteroaryl ring; and
R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring, for example, R¹⁰¹ is NH₂, NH(C₁₋₃₀ alkyl), N(CH₃)(C₁₋₃₀ alkyl),

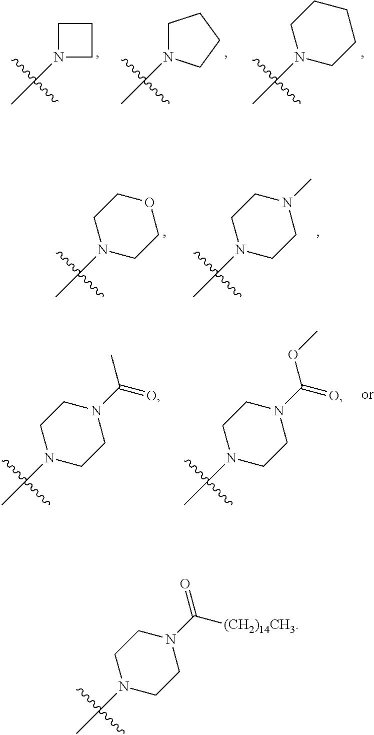

11. The compound of claim 10, or a pharmaceutically acceptable salt thereof, wherein R¹⁰¹ is NR³²R³³, wherein R³² and R³³ are independently hydrogen, a C₁₋₄ alkyl, or R³² and R³³ together with the N they are both attached to are joined to form an optionally substituted 4-8 membered monocyclic heterocyclic ring having one or two ring heteroatoms.

12. The compound of claim 10 or 11, or a pharmaceutically acceptable salt thereof, wherein R¹⁰¹ is NR³²R³³, wherein R³² and R³³ together with the N they are both attached to are joined to form a ring selected from 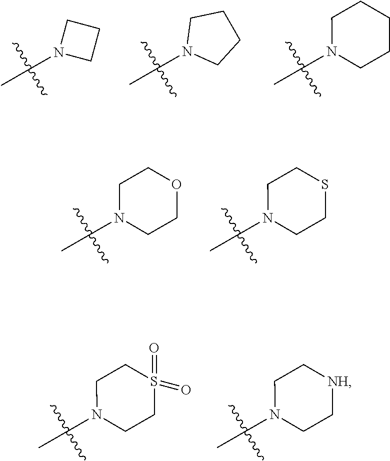
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

13. The compound of claim 10, or a pharmaceutically acceptable salt thereof, wherein R¹⁰¹ is a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted, e.g., with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

14. The compound of claim 10, or a pharmaceutically acceptable salt thereof, wherein R¹⁰¹ is a monocyclic ring selected from the following: 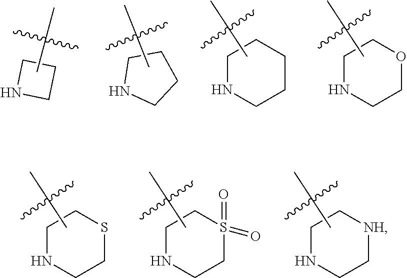
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

15. The compound of claim 10, or a pharmaceutically acceptable salt thereof, wherein R¹⁰¹ is a bicyclic ring selected from the following: 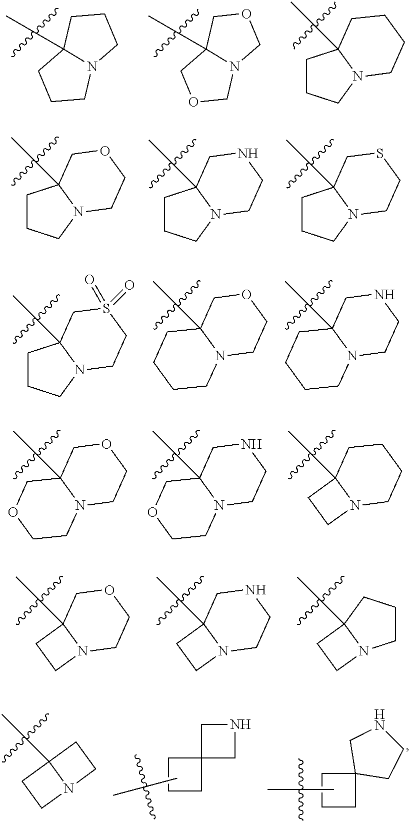
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

16. The compound of any one of claims 10-15, or a pharmaceutically acceptable salt thereof, wherein the —C₁₋₆ alkylene-unit in R²⁰ is selected from —CH₂—, —CH₂—CH₂—, —CH₂—CH₂—CH₂—, 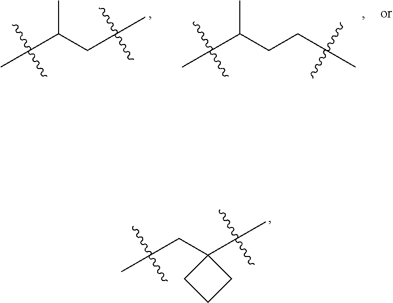
or the —C₁₋₆ alkylene-unit in R²⁰ is

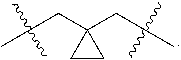 [SMILES: *CC1(C*)CC1]

17. The compound of any one of claims 1-9, or a pharmaceutically acceptable salt thereof, wherein R¹ in Formula I or A is 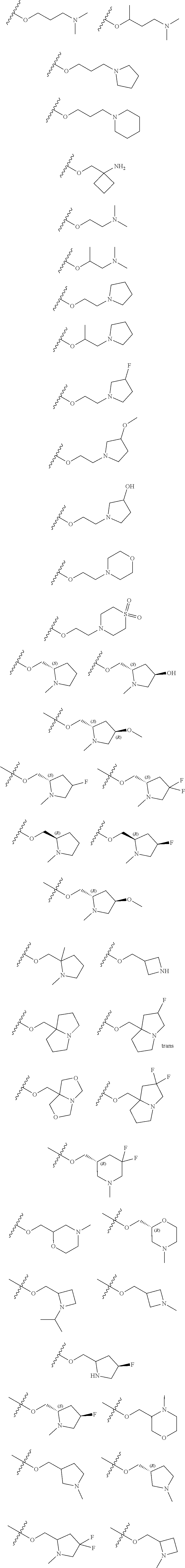
or R¹ in Formula I or A is 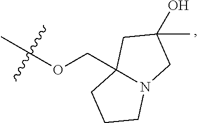 [SMILES: *OCC12CCCN1CC(C)(O)C2]
or R¹ in Formula I or A is methoxy, 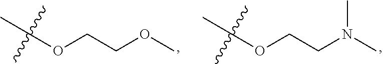
NH₂, NH(CH₃), or N (CH₃)₂,
or R¹ in Formula I or A is 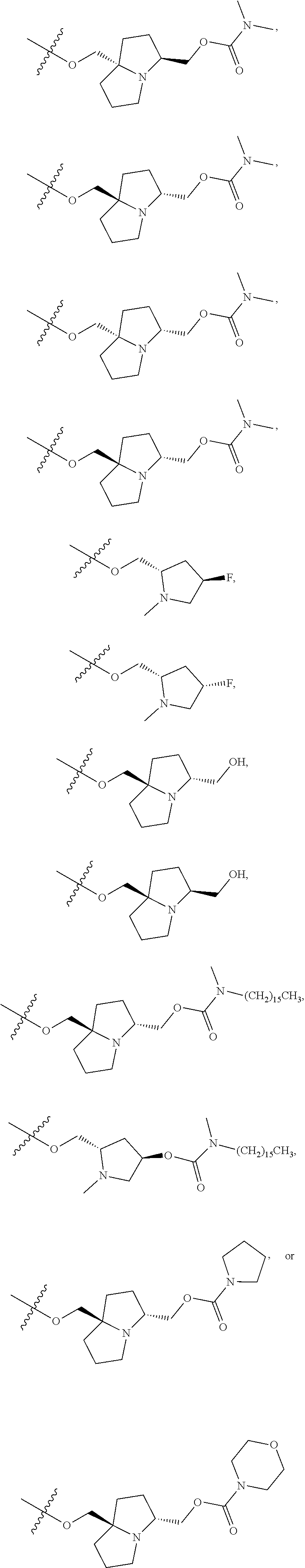
or R¹ in Formula I or A is 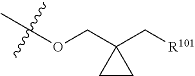
and R¹⁰¹ is NH₂, NH(C₁₋₃₀ alkyl), N(CH₃)(C₁₋₃₀ alkyl)

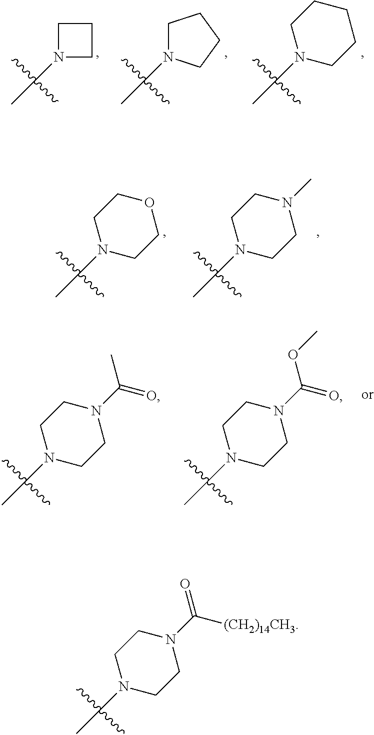

18. The compound of any one of claims 1-9, or a pharmaceutically acceptable salt thereof, wherein R in Formula I or A is OR²⁰, wherein R²⁰ is an optionally substituted C₃₋₆ carbocyclic ring or 4-10 membered heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted, e.g., with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

19. The compound of claim 18, or a pharmaceutically acceptable salt thereof, wherein R²⁰ is a monocyclic ring selected from the following: 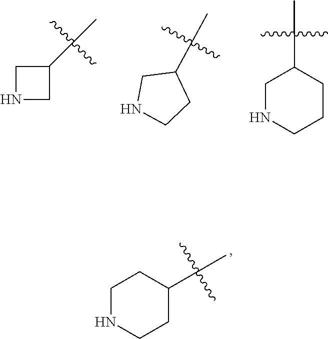 [SMILES: *C1CNC1 ; *C1CCNC1 ; *C1CCCNC1 ; *C1CCNCC1]
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, tetrahydropyranyl, —N(CH₃)₂, —OH, and —OCH₃.

20. The compound of any one of claims 1-9, or a pharmaceutically acceptable salt thereof, wherein R¹ in Formula I or A is selected from

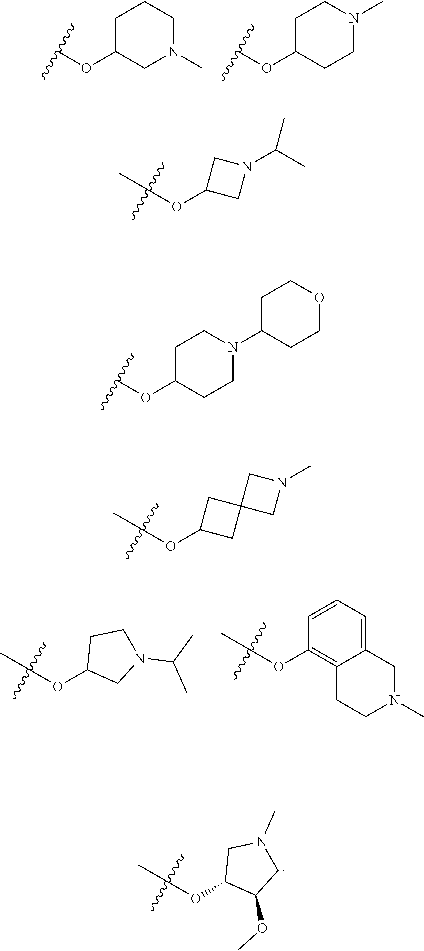

21. The compound of any one of claims 1-9, or a pharmaceutically acceptable salt thereof, wherein R¹ in Formula I or A is NR³⁰R³¹ or —C₁₋₆ alkylene-NR³⁰R³¹,
wherein R³⁰ and R³¹ are independently hydrogen, an optionally substituted C₁₋₆ alkyl, or an optionally substituted heterocyclic ring; or R³⁰ and R³¹ together with the N they are both attached to are joined to form an optionally substituted heterocyclic ring having one or two ring heteroatoms.

22. The compound of claim 21, or a pharmaceutically acceptable salt thereof, wherein R³⁰ and R³¹ together with the N they are both attached to are joined to form a ring selected from 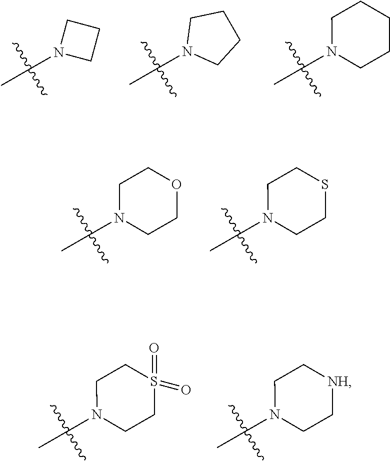
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

23. The compound of any one of claims 1-9, or a pharmaceutically acceptable salt thereof, wherein R¹ in Formula I or A is selected from

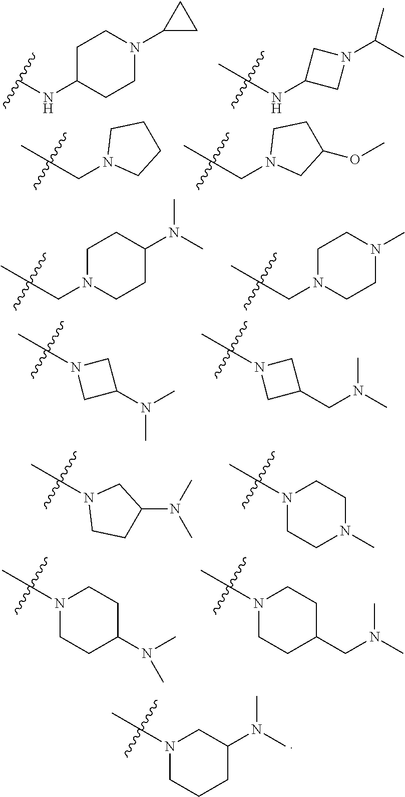

24. The compound of any one of claims 1-9, or a pharmaceutically acceptable salt thereof, wherein R¹ in Formula I or A is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted, e.g., with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

25. The compound of claim 24, or a pharmaceutically acceptable salt thereof, wherein R¹ in Formula I or A is selected from 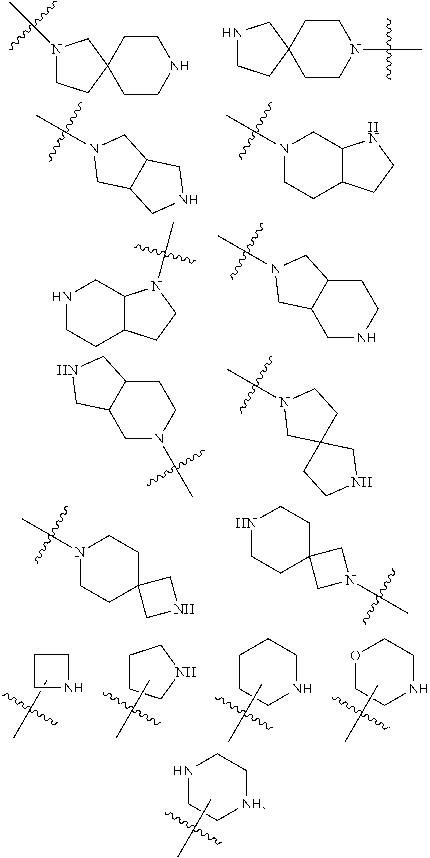
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

26. The compound of claim 24, or a pharmaceutically acceptable salt thereof, wherein R¹ in Formula I or A is selected from

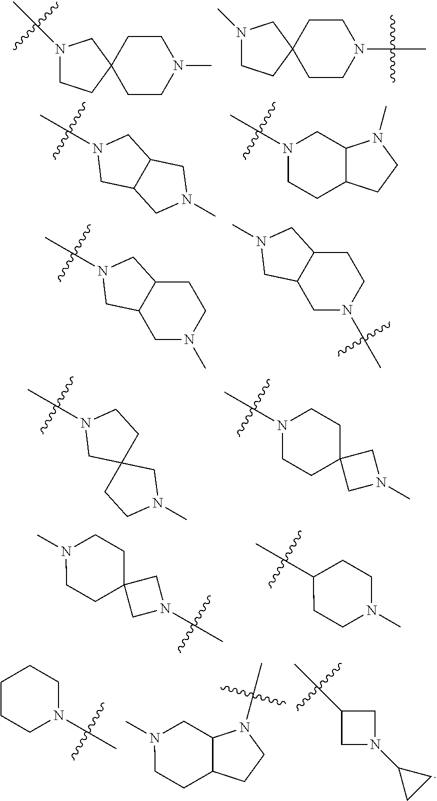

27. The compound of any one of claims 1-9, or a pharmaceutically acceptable salt thereof, wherein R¹ in Formula I or A has a structure of F-1: 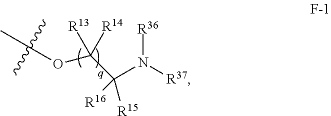
wherein:
R¹³ and R¹⁴ at each occurrence are independently hydrogen or a C₁₋₄ alkyl,
q is an integer of 0-6,
R¹⁵, R¹⁶, R³⁶, and R³⁷, together with the intervening carbon and nitrogen atoms, form an optionally substituted 6-10 membered fused bicyclic ring.

28. The compound of claim 27, or a pharmaceutically acceptable salt thereof, wherein q is 1.

29. The compound of claim 27, or a pharmaceutically acceptable salt thereof, wherein q is 2.

30. The compound of any one of claims 27-29, or a pharmaceutically acceptable salt thereof, wherein R¹³ and R¹⁴ at each occurrence are independently hydrogen or methyl.

31. The compound of any one of claims 27-30, or a pharmaceutically acceptable salt thereof, wherein R¹⁵, R¹⁶, R³⁶, and R³⁷, together with the intervening carbon and nitrogen atoms, form an optionally substituted 6-10 membered fused bicyclic ring 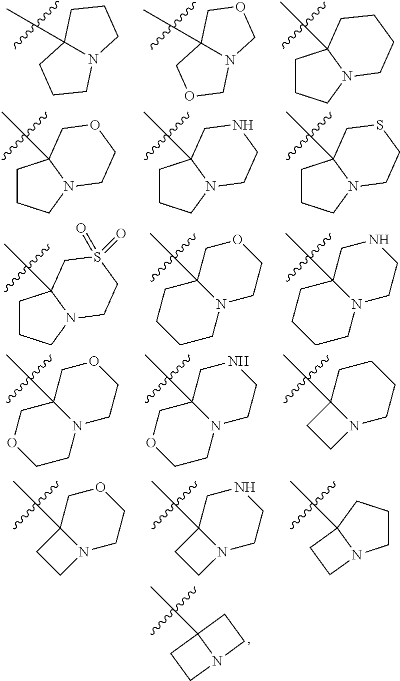
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

32. The compound of any one of claims 27-30, or a pharmaceutically acceptable salt thereof, wherein R¹⁵, R¹⁶, R³⁶, and R³⁷, together with the intervening carbon and nitrogen atoms, form 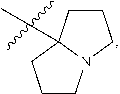 [SMILES: *C12CCCN1CCC2]
which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

33. The compound of any one of claims 27-30, or a pharmaceutically acceptable salt thereof, wherein R¹ in Formula I or A is selected from

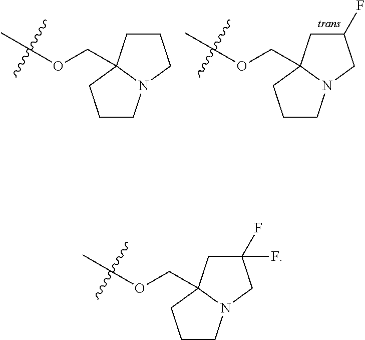

34. The compound of any one of claims 1-33, or a pharmaceutically acceptable salt thereof, wherein R² in Formula I or A is -(L²)ₘ₂-R¹⁰², wherein
m2 is 0 or 1, and when m2 is 1, L² is CH₂, O, NH, or NCH₃,
R¹⁰² is an optionally substituted 4-10 membered heterocyclic or heteroaryl ring having one or two ring nitrogen atoms.

35. The compound of claim 34, or a pharmaceutically acceptable salt thereof, wherein m2 is 0 or 1, and R¹⁰² is an optionally substituted 4-10 membered heterocyclic ring having one or two ring nitrogen atoms, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted.

36. The compound of claim 35, or a pharmaceutically acceptable salt thereof, wherein R¹⁰² is selected from the following ring structures: 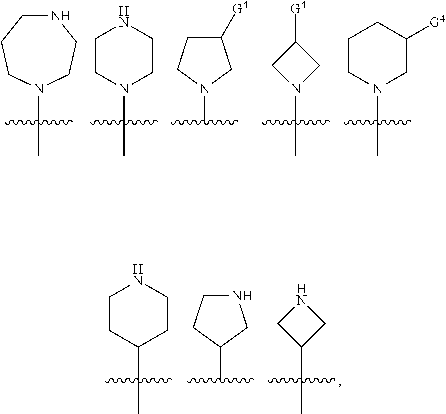
wherein G⁴ is -(L³)ₘ₃-NH₂, -(L³)ₘ₃-NH(C₁₋₄ alkyl), wherein m3 is 0 or 1, and when m3 is 1, L³ is C₁₋₄ alkylene, or G4 and one substituent on the ring are joined together to form a 4-6 membered heterocyclic ring having one or two ring nitrogen atoms;
and wherein each of the ring structures is optionally substituted with 1-3 (typically 1 or 2) substituents independently selected from C₁₋₄ alkyl, fluorine substituted C₁₋₄ alkyl, hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure.

37. The compound of claim 35, or a pharmaceutically acceptable salt thereof, wherein R¹⁰² is selected from: 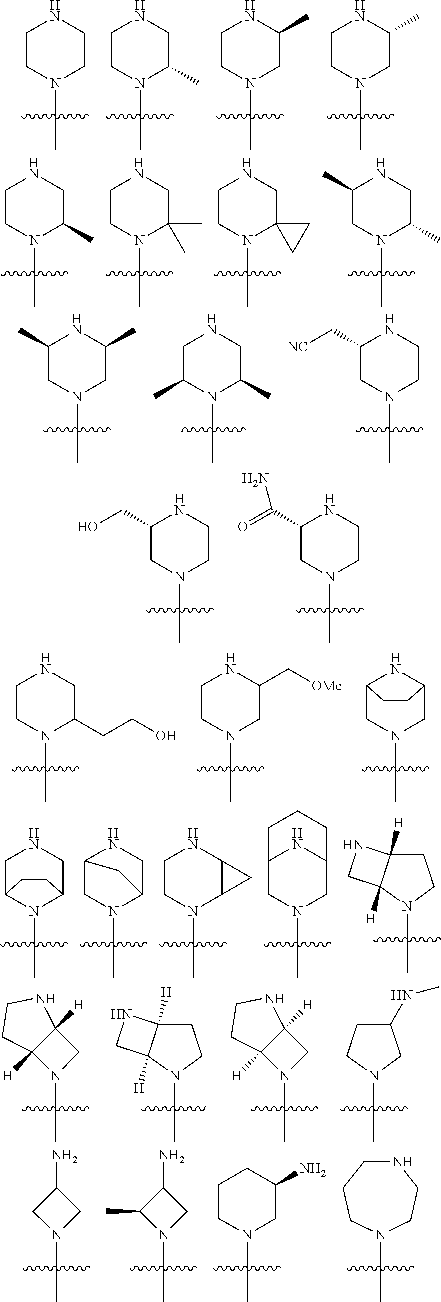 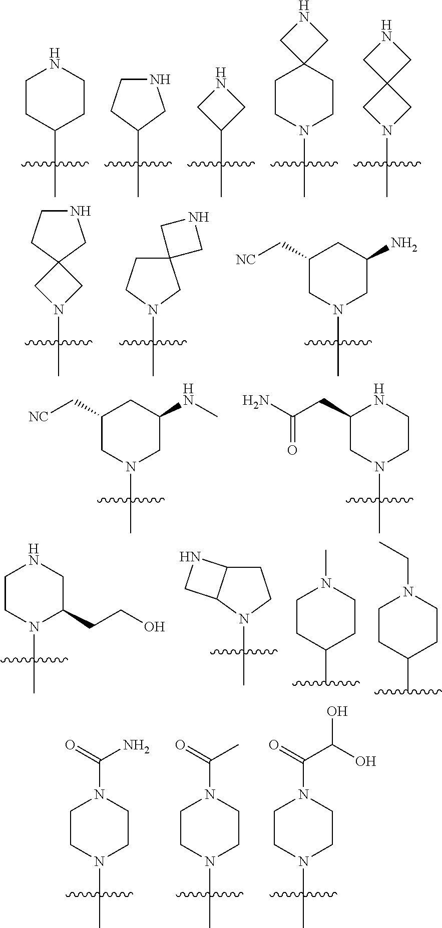
or R¹⁰² is 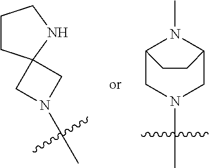
or R¹⁰² is

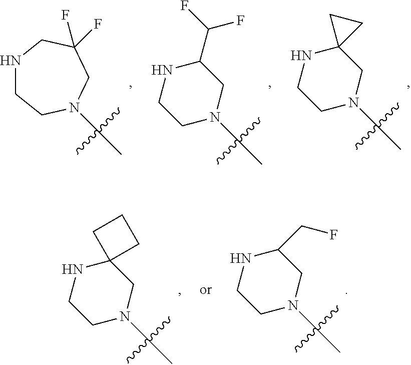 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

38. The compound of claim 34, or a pharmaceutically acceptable salt thereof, wherein m2 is 1, L² is CH₂ or NH, and R¹⁰² is an optionally substituted 4-8 membered heterocyclic ring.

39. The compound of any one of claims 1-33, or a pharmaceutically acceptable salt thereof, wherein R² in Formula I or A is selected from

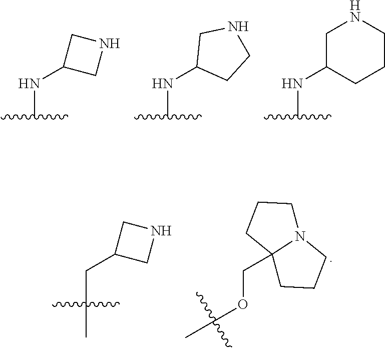 [SMILES: *NC1CNC1 ; *NC1CCNC1 ; *NC1CCCNC1 ; *CC1CNC1 ; *OCC12CCCN1CCC2]

40. The compound of any one of claims 1-33, or a pharmaceutically acceptable salt thereof, wherein R² in Formula I or A is -(L²)ₘ₂-R¹⁰², wherein m2 is 0 or 1, and when m2 is 1, L² is CH₂, O, NH, or NCH₃, wherein R¹⁰² is an optionally substituted C₃₋₇ carbocyclic, optionally substituted phenyl, or optionally substituted 5 or 6 membered heteroaryl ring, each of which has at least one nitrogen containing substituent, e.g., NH₂, NH(C₁₋₄ alkyl) or N(C₁₋₄ alkyl)(C₁₋₄ alkyl).

41. The compound of claim 40, or a pharmaceutically acceptable salt thereof, wherein R² in Formula I or A is selected from

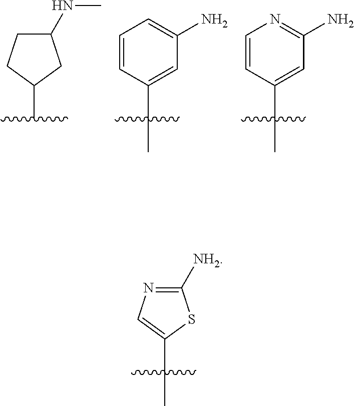

42. The compound of any one of claims 1-33, or a pharmaceutically acceptable salt thereof, wherein R² in Formula I or A is 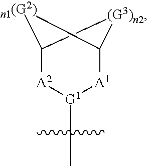
wherein:
G¹ is CR¹⁷ or N;
each occurrence of G² and G³ is independently CR¹⁸R¹⁹, O, or NR³⁸, provided that at least one instance of G² and G³ is NR³⁸;
n1 and n2 are each independently an integer of 1, 2, 3, or 4;
A¹ and A² are each independently a bond, CR¹⁸R¹⁹, O, or NR³⁸, provided that at least one of A¹ and A² is not O or NR³⁸,
wherein: each occurrence of R¹⁷, R¹⁸, or R¹⁹ is independently hydrogen, F, —OH, or an optionally substituted C₁₋₆ alkyl, or R¹⁸ and R¹⁹ together with the carbon they are both attached to are joined to form an oxo or imino group or a ring; and
R³⁸ at each occurrence is independently hydrogen, a nitrogen protecting group, or an optionally substituted C₁₋₆ alkyl.

43. The compound of claim 42, or a pharmaceutically acceptable salt thereof, wherein: G¹ is CH or N.

44. The compound of claim 42 or 43, or a pharmaceutically acceptable salt thereof, wherein A¹ and A² are each independently a bond or CH₂.

45. The compound of claim 42 or 43, or a pharmaceutically acceptable salt thereof, wherein A¹ and A² are both a bond or both CH₂.

46. The compound of any one of claims 42-45, or a pharmaceutically acceptable salt thereof, wherein each occurrence of G² is independently CR¹⁸R¹⁹.

47. The compound of any one of claims 42-46, or a pharmaceutically acceptable salt thereof, wherein n1 is 1, 2, or 3.

48. The compound of any one of claims 42-47, or a pharmaceutically acceptable salt thereof, wherein one instance of G³ is NH or N—(C₁₋₄ alkyl).

49. The compound of any one of claims 42-48, or a pharmaceutically acceptable salt thereof, wherein n2 is 1, 2, or 3.

50. The compound of claim 42, or a pharmaceutically acceptable salt thereof, wherein R² in Formula I or A is selected from the following: 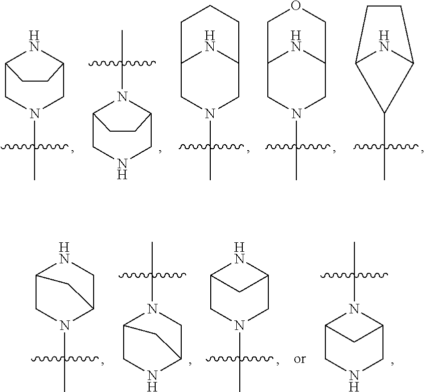
or R² in Formula I or A is

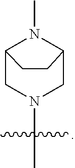 [SMILES: *N1CC2CCC(C1)N2C]

51. The compound of any one of claims 1-50, wherein R³ in Formula I or A is (1) a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂; or (2) a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂.

52. The compound of any one of claims 1-50, wherein R³ in Formula I or A is selected from: 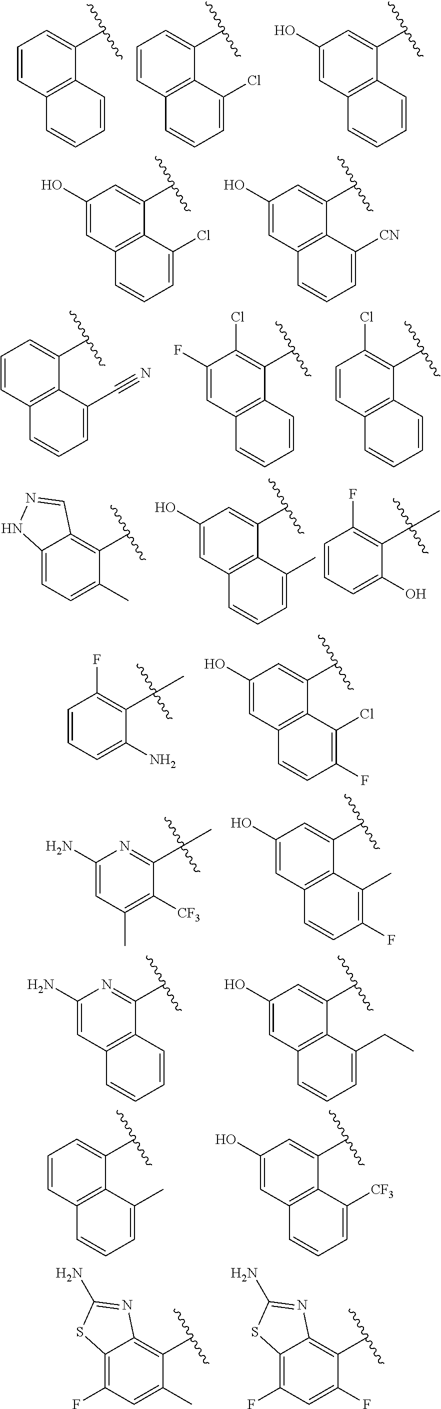 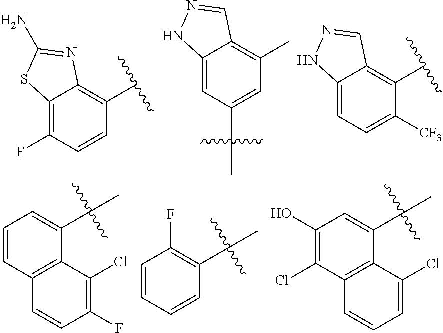
or R³ in Formula I or A is selected from 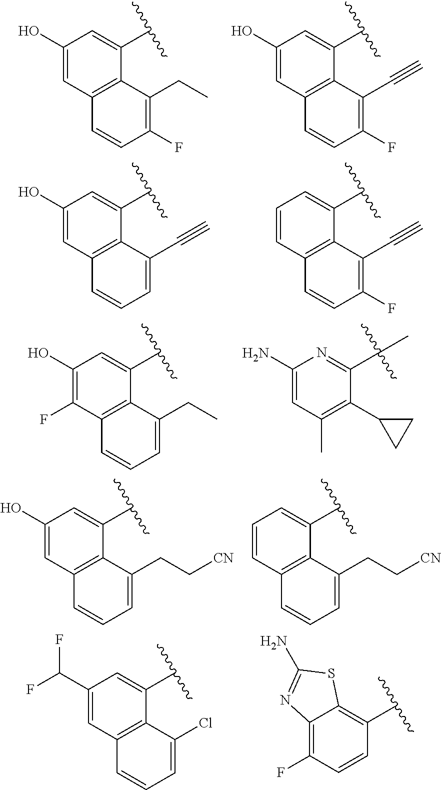
or R³ in Formula I or A is: 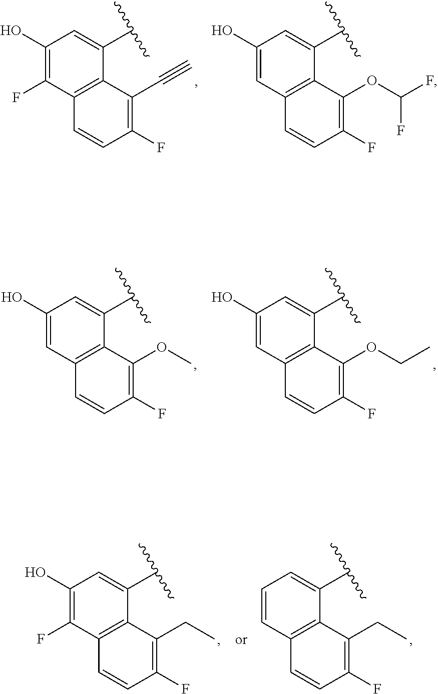 [SMILES: *c1cc(O)c(F)c2ccc(F)c(C#C)c12 ; *c1cc(O)cc2ccc(F)c(OC(F)F)c12 ; *c1cc(O)cc2ccc(F)c(OC)c12 ; *c1cc(O)cc2ccc(F)c(OCC)c12 ; *c1cc(O)c(F)c2ccc(F)c(CC)c12 ; *c1cccc2ccc(F)c(CC)c12]
or R³ in Formula I or A is: 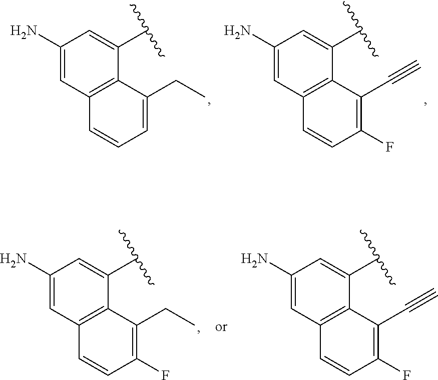 [SMILES: *c1cc(N)cc2cccc(CC)c12 ; *c1cc(N)cc2ccc(F)c(C#C)c12 ; *c1cc(N)cc2ccc(F)c(CC)c12 ; *c1cc(N)cc2ccc(F)c(C#C)c12]
or R³ in Formula I or A is:

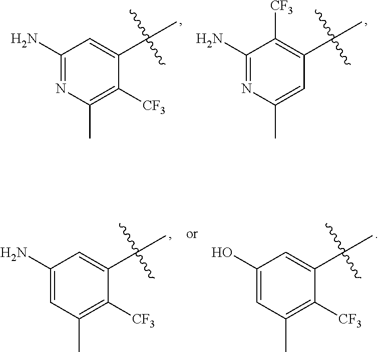 [SMILES: *c1cc(N)nc(C)c1C(F)(F)F ; *c1cc(C)nc(N)c1C(F)(F)F ; *c1cc(N)cc(C)c1C(F)(F)F ; *c1cc(O)cc(C)c1C(F)(F)F]

53. A compound of Formula II, or a pharmaceutically acceptable salt thereof: 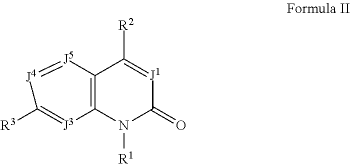
wherein:
J¹ is CR⁹ or N;
J³ is CR¹¹ or N;
J⁴ is CR¹² or N;
J⁵ is CR^{12A} or N;
R¹ is hydrogen, an optionally substituted C₁₋₆ alkyl, an optionally substituted carbocyclic ring, an optionally substituted aryl, -(L¹)ₘ₁-OR²⁰, -(L¹)ₘ₁-NR³⁰R³¹, or an optionally substituted heterocyclic or heteroaryl ring;
R² is a ring or ring-chain structure which has a basic functional group with a pKa of the conjugated acid of about 6 or higher, or an acylated derivative thereof (i.e., the basic functional group, such as a basic NH, is bonded with an acyl group),
R³ is an optionally substituted aryl or an optionally substituted heteroaryl,
R⁹ is hydrogen, halogen, cyano, optionally substituted C₁₋₄ alkyl (e.g., methyl, ethyl, CF₃, etc.), optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S, R¹¹, R¹² and R^{12A} at each occurrence is independently hydrogen, F, Cl, Br, I, CN, —OH, —C(O)NH₂,
—C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, or cyclobutoxy; and
wherein:
m1 is 0 or 1, and when m1 is 1, L¹ is an optionally substituted alkylene, an optionally substituted carbocyclylene, an optionally substituted heterocyclylene;
R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring; or one of R³⁰ and R³¹ together with a suitable atom of L¹ and any intervening atoms form an optionally substituted heterocyclic or heteroaryl ring.

54. The compound of claim 53, or a pharmaceutically acceptable salt thereof, wherein when present, R¹¹ in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl.

55. The compound of claim 53 or 54, or a pharmaceutically acceptable salt thereof, wherein when present, R¹² in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl, and wherein when present, R^{12A} in Formula II is hydrogen, F, Cl, —CN, —OH, methoxy, ethoxy, —O—CH₂-cyclopropyl, —C(O)NHMe, CF₃, methyl, ethyl, isopropyl, or cyclopropyl, preferably, when present, R^{12A} in Formula II is hydrogen, methyl, Cl, or methoxy.

56. The compound of any one of claims 53-55, or a pharmaceutically acceptable salt thereof, wherein R¹ in Formula II is —C₁₋₆ alkylene-NR³⁰R³¹,
wherein R³⁰ and R³¹ are independently hydrogen, an optionally substituted C₁₋₆ alkyl, or an optionally substituted heterocyclic ring; or R³⁰ and R³¹ together with the N they are both attached to are joined to form an optionally substituted heterocyclic ring having one or two ring heteroatoms, or one of R³⁰ and R³¹ together with a CH₂ unit of the C₁₋₆ alkylene and any intervening atoms form an optionally substituted heterocyclic or heteroaryl ring having one or two ring heteroatoms.

57. The compound of any one of claims 53-55, or a pharmaceutically acceptable salt thereof, wherein R¹ is —C₁₋₆ alkylene-NR³⁰R³¹,
wherein R³⁰ together with a CH₂ unit of the C₁₋₆ alkylene and any intervening atoms form a ring selected from (R³¹ is shown): 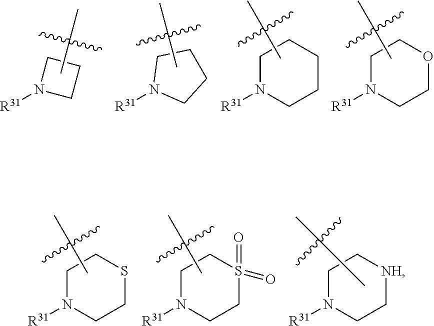
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

58. The compound of claim 57, wherein R³¹ is —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₚ-cyclopropyl, —(CH₂)ₚ-cyclobutyl, or —(CH₂)ₚ-(4-6 membered heterocyclic ring having 1 or 2 ring heteroatoms independently selected from O, N, and S), wherein x is 1, 2, or 3, and p is 0, 1, 2, or 3.

59. The compound of any one of claims 53-55, or a pharmaceutically acceptable salt thereof, wherein R¹ is —C₁₋₆ alkylene-NR³⁰R³¹,
wherein R³⁰ and R³¹ together with the N they are both attached to are joined to form a ring selected from 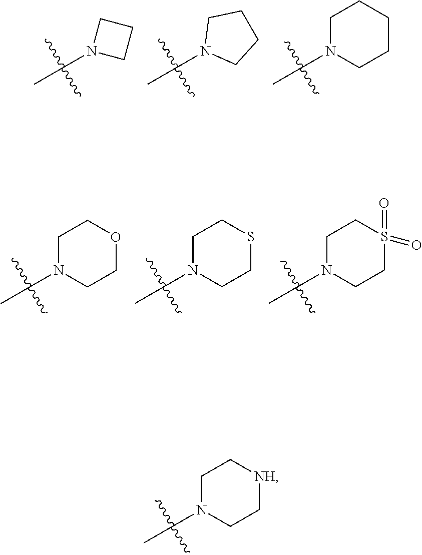
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

60. The compound of any one of claims 53-55, or a pharmaceutically acceptable salt thereof, wherein R¹ is

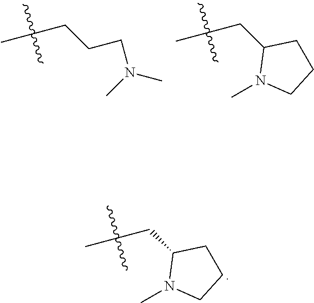

61. The compound of any one of claims 53-60, or a pharmaceutically acceptable salt thereof, wherein R² is selected from 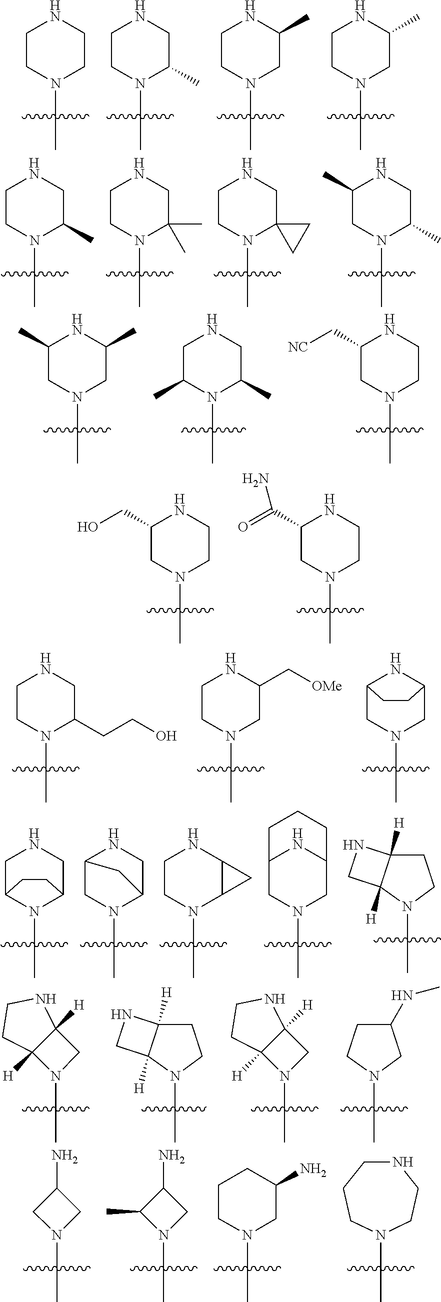 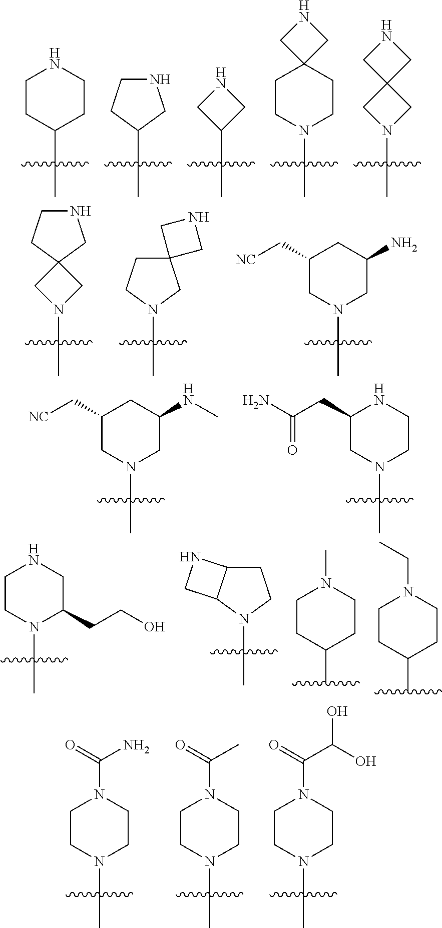
or R² is 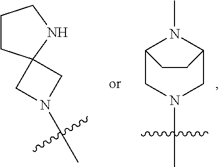
or R² is

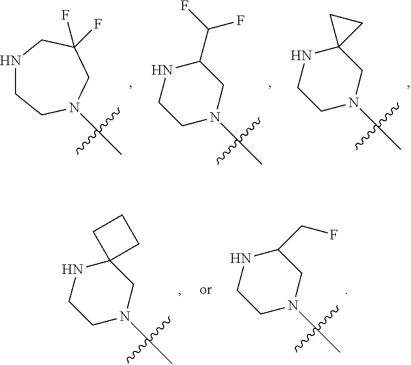 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

62. The compound of any one of claims 53-61, or a pharmaceutically acceptable salt thereof, wherein R³ is (1) a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂ or (2) a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂.

63. The compound of any one of claims 53-61, or a pharmaceutically acceptable salt thereof, wherein R³ is selected from: 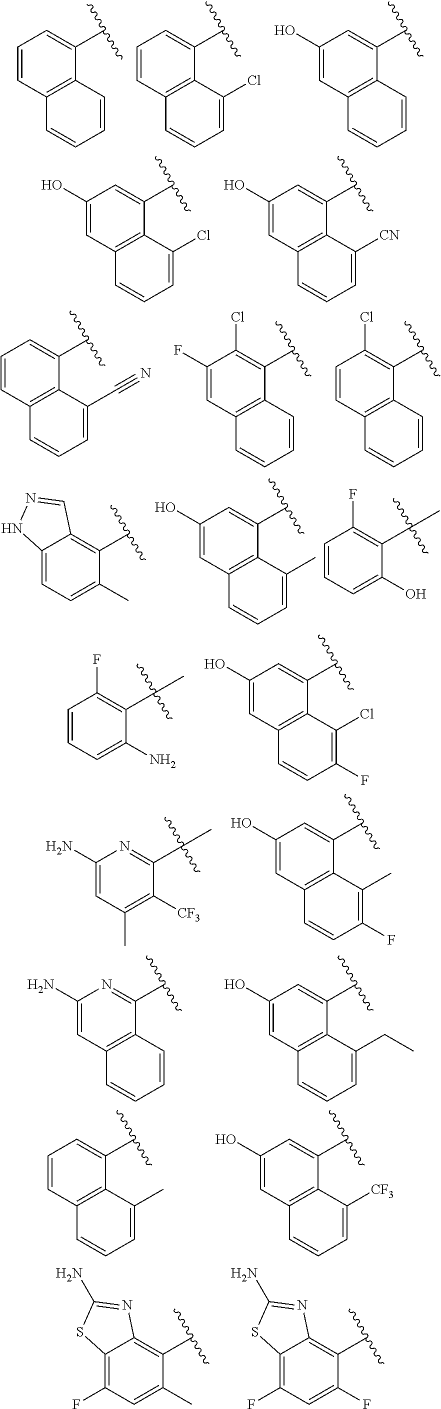 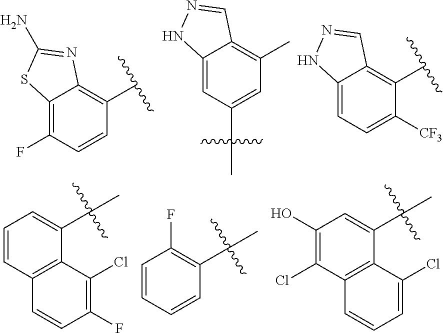
or R³ is selected from: 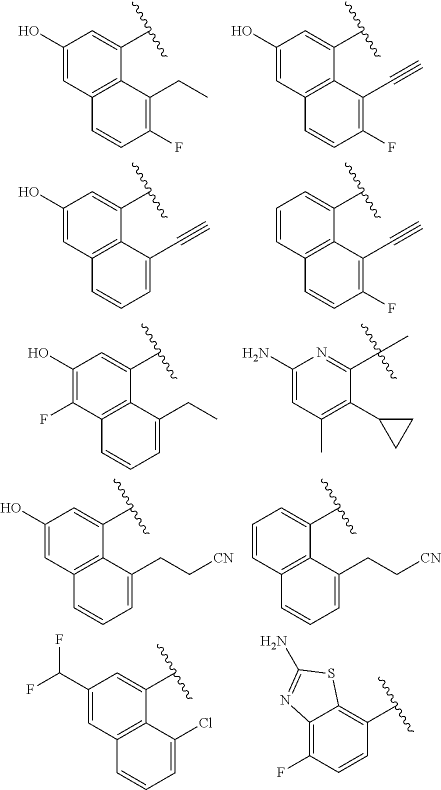
or R³ is: 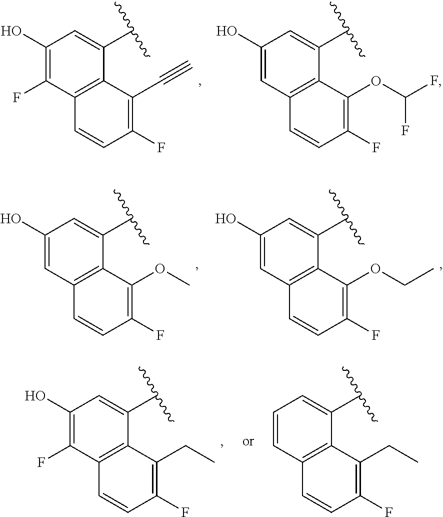 [SMILES: *c1cc(O)c(F)c2ccc(F)c(C#C)c12 ; *c1cc(O)cc2ccc(F)c(OC(F)F)c12 ; *c1cc(O)cc2ccc(F)c(OC)c12 ; *c1cc(O)cc2ccc(F)c(OCC)c12 ; *c1cc(O)c(F)c2ccc(F)c(CC)c12 ; *c1cccc2ccc(F)c(CC)c12]
or R³ is: 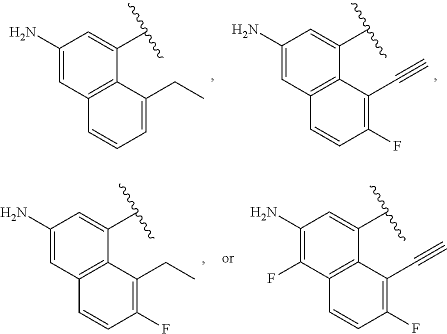 [SMILES: *c1cc(N)cc2cccc(CC)c12 ; *c1cc(N)cc2ccc(F)c(C#C)c12 ; *c1cc(N)cc2ccc(F)c(CC)c12 ; *c1cc(N)c(F)c2ccc(F)c(C#C)c12]
or R³ is:

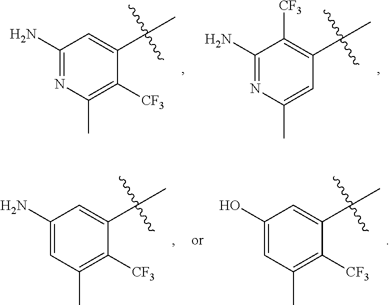 [SMILES: *c1cc(N)nc(C)c1C(F)(F)F ; *c1cc(C)nc(N)c1C(F)(F)F ; *c1cc(N)cc(C)c1C(F)(F)F ; *c1cc(O)cc(C)c1C(F)(F)F]

64. A compound of Formula III, or a pharmaceutically acceptable salt thereof: 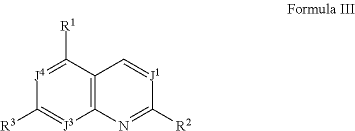
wherein:
J¹ is CR⁹ or N;
J³ is CR¹¹ or N;
J⁴ is CR¹² or N;
R¹ is hydrogen, -(L¹)ₘ₁-OR²⁰, halogen, -(L¹)ₘ₁-NR³⁰R³¹, or an optionally substituted heterocyclic or heteroaryl ring;
R² is a ring or ring-chain structure which has a basic functional group with a pKa of the conjugated acid of about 6 or higher, or an acylated derivative thereof (i.e., the basic functional group, such as a basic NH, is bonded with an acyl group),
R³ is an optionally substituted aryl or an optionally substituted heteroaryl,
R⁹ is hydrogen, halogen, cyano, optionally substituted C₁₋₄ alkyl (e.g., methyl, ethyl, CF₃, etc.), optionally substituted C₁₋₄ alkoxy, optionally substituted C₃₋₆ cycloalkyl, optionally substituted aryl, optionally substituted 4-8 membered heterocyclyl having 1-4 heteroatoms independently selected from N, O, and S, or optionally substituted 5-10 membered heteroaryl having 1-4 heteroatoms independently selected from N, O, and S,
R¹¹ and R¹² at each occurrence is independently hydrogen, F, Cl, Br, I, CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, or cyclobutoxy; and
wherein:
m1 is 0 or 1, and when m1 is 1, L¹ is an optionally substituted alkylene, an optionally substituted carbocyclylene, an optionally substituted heterocyclylene;
R²⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring;
R³⁰ and R³¹ are independently hydrogen, a nitrogen protecting group, an optionally substituted C₁₋₆ alkyl, an optionally substituted carbocyclic ring, or an optionally substituted heterocyclic ring; or R³⁰ and R³¹ are joined to form an optionally substituted heterocyclic or heteroaryl ring; or one of R³⁰ and R³¹ together with a suitable atom of L¹ and any intervening atoms form an optionally substituted heterocyclic or heteroaryl ring.

65. The compound of claim 64, or a pharmaceutically acceptable salt thereof, wherein when present, R¹¹ in Formula III is hydrogen, F, Cl, Br, —CN, —OH, methoxy, ethoxy, —O—CH₂-cyclopropyl, —C(O)NHMe, CF₃, methyl, ethyl, isopropyl, or cyclopropyl, and when present, R¹² in Formula III is hydrogen, F, Cl, —CN, —OH, methoxy, ethoxy, —O—CH₂-cyclopropyl, —C(O)NHMe, CF₃, methyl, ethyl, isopropyl, or cyclopropyl.

66. The compound of claim 64 or 65, or a pharmaceutically acceptable salt thereof, wherein R¹ is an optionally substituted heterocyclic ring, preferably, a monocyclic 4-8 membered heterocyclic ring having 1 or 2 ring heteroatoms independently selected from N, O, and S, or a fused, bridged or spiro bicyclic 6-10 membered heterocyclic ring having one to three ring heteroatoms independently selected from N, O, and S, wherein the monocyclic or bicyclic ring is optionally substituted.

67. The compound of claim 66, or a pharmaceutically acceptable salt thereof, wherein R¹ is selected from 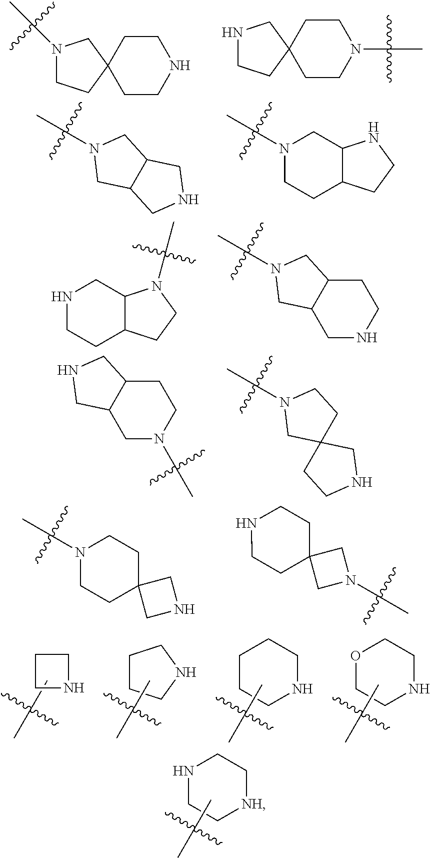
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

68. The compound of claim 64 or 65, or a pharmaceutically acceptable salt thereof, wherein R¹ is —OR²⁰, wherein R²⁰ is a —C₁₋₆ alkylene-R¹⁰¹, wherein R¹⁰ is NR³²R³³ or an optionally substituted 4-10 membered heterocyclic ring,
wherein the C₁₋₆ alkylene is optionally substituted, e.g., with one or more substituents independently selected from F, OH, NR³⁴R³⁵, and C₁₋₄ alkyl optionally substituted with 1-3 fluorine, or two substituents of the alkylene group are joined to form a ring;
R³² and R³³ are independently hydrogen, a nitrogen protecting group, an optionally substituted C₁₋₆ alkyl, an optionally substituted carbocyclic ring, or an optionally substituted heterocyclic ring; or R³² and R³³ are joined to form an optionally substituted heterocyclic or heteroaryl ring; and
R³⁴ and R³⁵ are independently hydrogen, a nitrogen protecting group, an optionally substituted C₁₋₆ alkyl, an optionally substituted carbocyclic ring, or an optionally substituted heterocyclic ring; or R³⁴ and R³⁵ are joined to form an optionally substituted heterocyclic or heteroaryl ring.

69. The compound of claim 68, or a pharmaceutically acceptable salt thereof, wherein R¹⁰¹ is a monocyclic ring selected from the following: 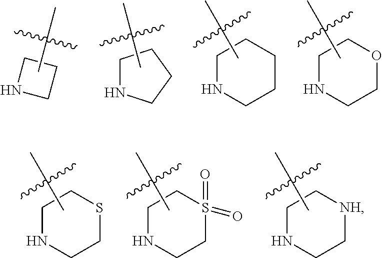
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

70. The compound of claim 68, or a pharmaceutically acceptable salt thereof, wherein R¹⁰¹ is a bicyclic ring selected from the following: 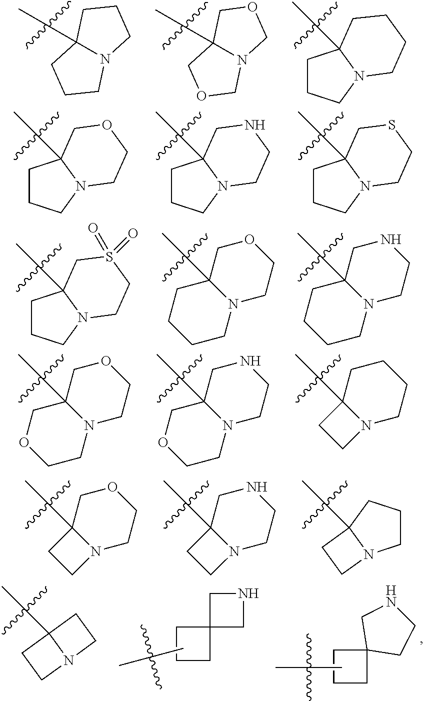
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

71. The compound of claim 64 or 65, or a pharmaceutically acceptable salt thereof, wherein R¹ is selected from:

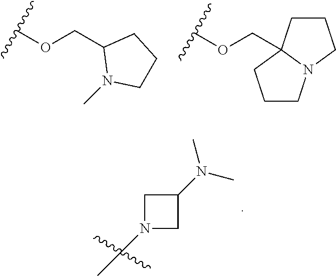

72. The compound of any one of claims 64-71, or a pharmaceutically acceptable salt thereof, wherein R² is -(L²)ₘ₂-R¹⁰², wherein
m2 is 0 or 1, and when m2 is 1, L² is CH₂, O, NH, or NCH₃,
R¹⁰² is an optionally substituted 4-10 membered heterocyclic or heteroaryl ring having one or two ring nitrogen atoms.

73. The compound of claim 72, or a pharmaceutically acceptable salt thereof, wherein m2 is 0, and R¹⁰² is an optionally substituted 4-10 membered heterocyclic ring having one or two ring nitrogen atoms.

74. The compound of claim 72 or 73, or a pharmaceutically acceptable salt thereof, wherein R¹⁰² is selected from: 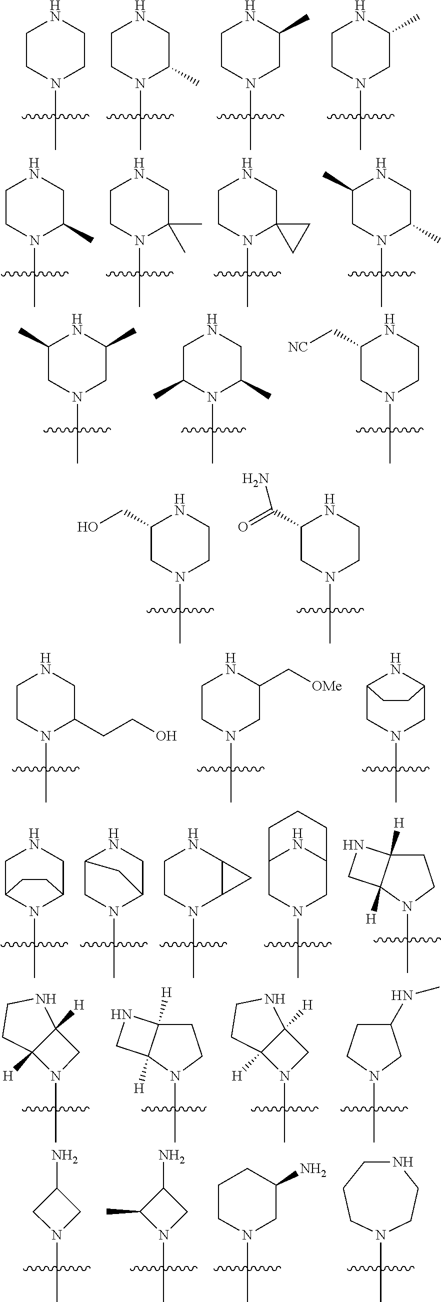 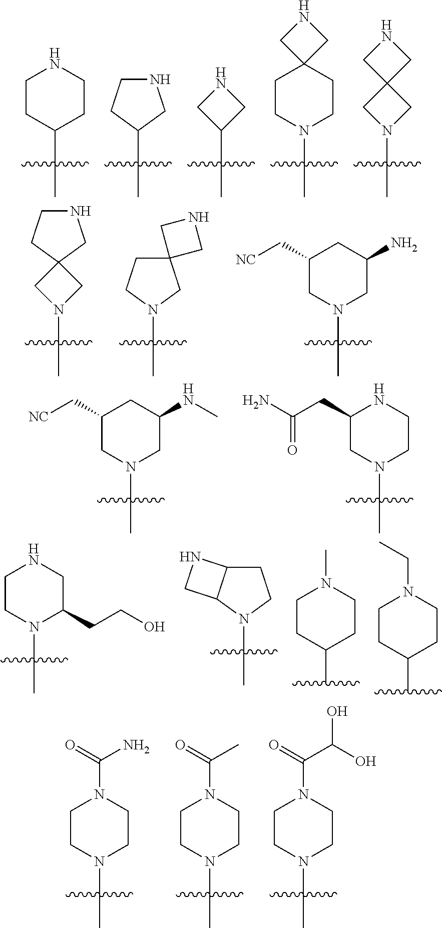
or R¹⁰² is 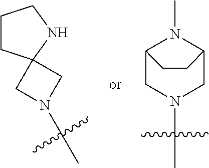
or R¹⁰² is

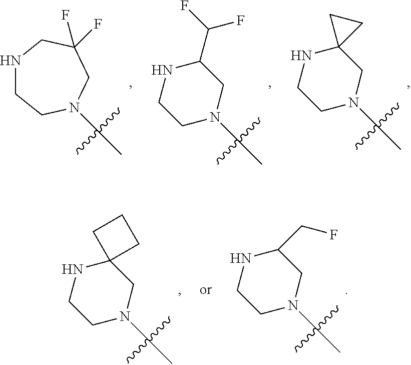 [SMILES: *N1CCNCC(F)(F)C1 ; *N1CCNC(C(F)F)C1 ; *N1CCNC2(CC2)C1 ; *N1CCNC2(CCC2)C1 ; *N1CCNC(CF)C1]

75. The compound of any one of claims 64-74, or a pharmaceutically acceptable salt thereof, wherein R³ is (1) a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂; or (2) a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl or propargyl), cyclopropyl, —NH₂, —CN, protected —OH, and a protected —NH₂.

76. The compound of any one of claims 64-74, or a pharmaceutically acceptable salt thereof, wherein R³ is selected from: 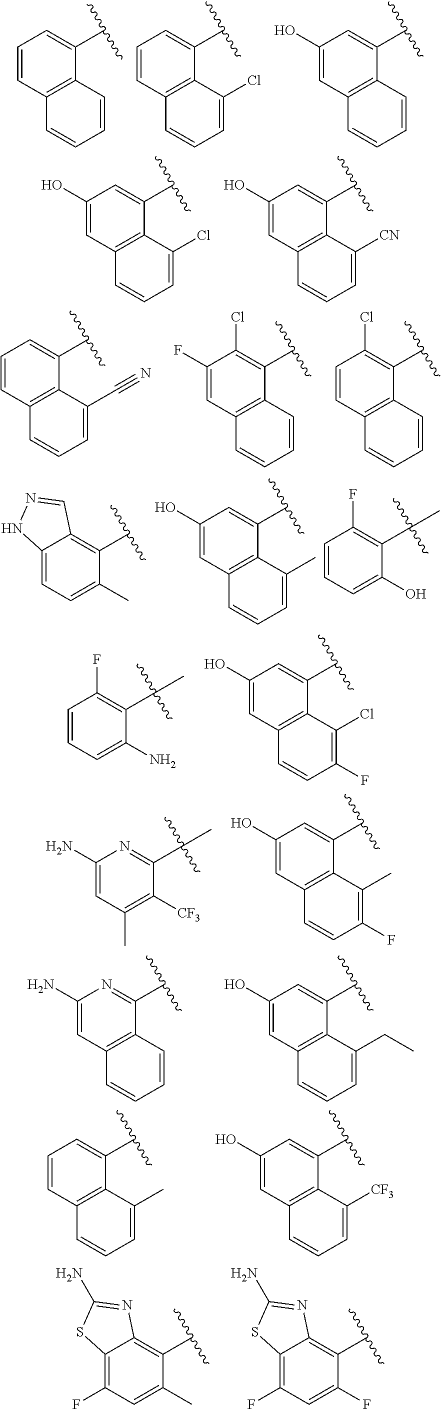 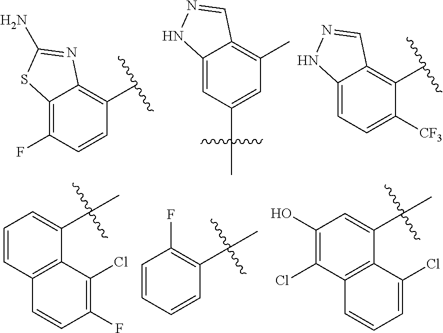
or R³ is selected from: 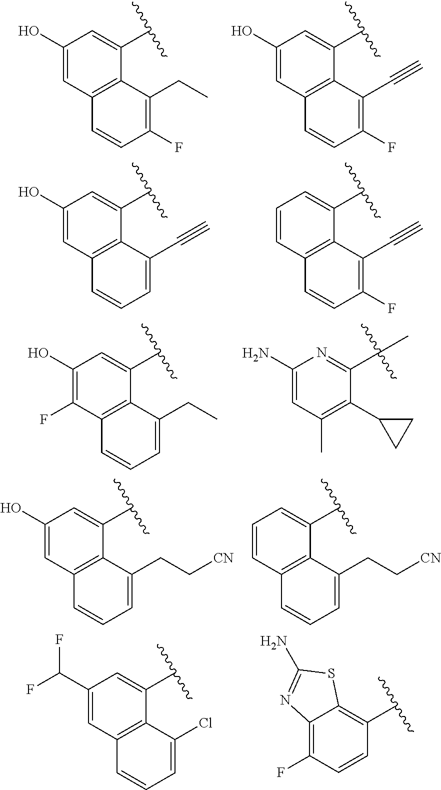
or R³ is: 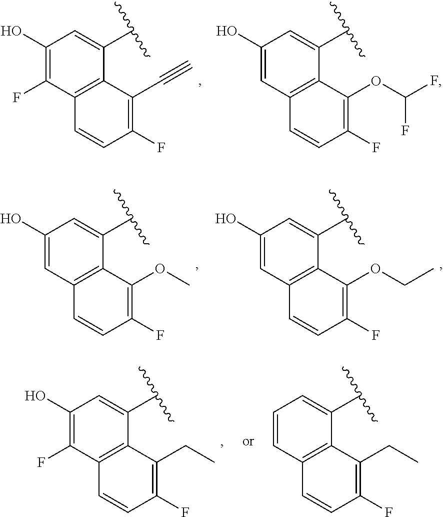 [SMILES: *c1cc(O)c(F)c2ccc(F)c(C#C)c12 ; *c1cc(O)cc2ccc(F)c(OC(F)F)c12 ; *c1cc(O)cc2ccc(F)c(OC)c12 ; *c1cc(O)cc2ccc(F)c(OCC)c12 ; *c1cc(O)c(F)c2ccc(F)c(CC)c12 ; *c1cccc2ccc(F)c(CC)c12]
or R³ is: 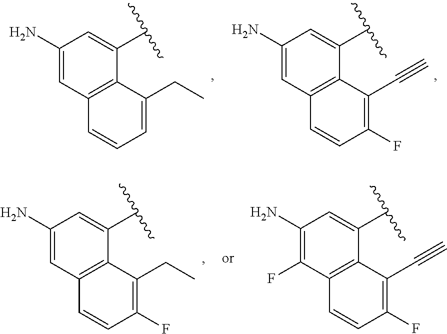 [SMILES: *c1cc(N)cc2cccc(CC)c12 ; *c1cc(N)cc2ccc(F)c(C#C)c12 ; *c1cc(N)cc2ccc(F)c(CC)c12 ; *c1cc(N)c(F)c2ccc(F)c(C#C)c12]
or R³ is:

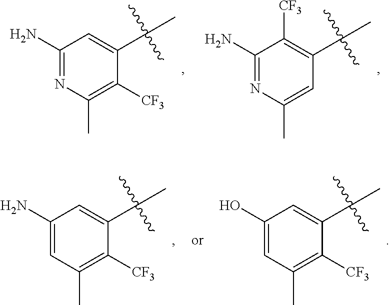 [SMILES: *c1cc(N)nc(C)c1C(F)(F)F ; *c1cc(C)nc(N)c1C(F)(F)F ; *c1cc(N)cc(C)c1C(F)(F)F ; *c1cc(O)cc(C)c1C(F)(F)F]

77. A compound selected from compound numbers 1-247, or a pharmaceutically acceptable salt thereof.

78. A pharmaceutical composition comprising the compound of any one of claims 1-77 or a pharmaceutically acceptable salt thereof and a pharmaceutically acceptable excipient.

79. A method of inhibiting KRAS mutant protein in a cancer cell, the method comprising contacting the cancer cell with the compound of any one of claims 1-77 or a pharmaceutically acceptable salt thereof.

80. A method of treating cancer in a subject, the method comprising administering to the subject a therapeutically effective amount of the compound of any one of claims 1-77 or a pharmaceutically acceptable salt thereof, or the pharmaceutical composition of claim 78.

81. The method of claim 80, wherein the cancer is pancreatic cancer, colorectal cancer, lung cancer, endometrial cancer, appendix cancer, cholangiocarcinoma, bladder urothelial cancer, ovarian cancer, gastric cancer, breast cancer, bile duct cancer and/or a hematologic malignancy.

82. The method of claim 80 or 81, further comprising treating the subject with an additional therapy (combination therapy).

83. The method of claim 82, wherein the additional therapy (combination therapy) is a targeted therapeutic agent, chemotherapeutic agent, therapeutic antibody, radiation, cell therapy, gene therapy, and/or immunotherapy.

84. The method of any one of claims 80-83, wherein the subject has a mutation of KRAS, HRAS and/or NRAS.

85. A method for inhibiting proliferation of a cell population, the method comprising contacting the cell population with the compound of any one of claims 1-77 or a pharmaceutically acceptable salt thereof.

86. The method of claim 85, wherein inhibition of proliferation is measured as a decrease in cell viability of the cancer cell population.

87. A method for treating a disease or disorder mediated by a Ras (KRAS, HRAS and/or NRAS) mutant protein in a subject in need thereof, the method comprising:
determining if the subject has a KRAS, HRAS and/or NRAS mutation; and if the subject is determined to have the KRAS, HRAS and/or NRAS mutation, then administering to the subject a therapeutically effective amount of the compound of any one of claims 1-77 or a pharmaceutically acceptable salt thereof, or the pharmaceutical composition of claim 78.

88. The method of claim 87, wherein the disease or disorder is cancer, for example pancreatic cancer, colorectal cancer, lung cancer (e.g., non-small cell lung cancer), endometrial cancer, appendix cancer, cholangiocarcinoma, bladder urothelial cancer, ovarian cancer, gastric cancer, breast cancer, bile duct cancer and/or a hematologic malignancy.

89. A method for inhibiting cancer metastasis or tumor metastasis, the method comprising administering an effective amount of the compound of any one of claims 1-77 or a pharmaceutically acceptable salt thereof, or the pharmaceutical composition of claim 78 to a subject in need thereof.

90. The method of claim 88 or 89, further comprising treating the subject with an additional therapy (combination therapy), wherein the additional therapy is a targeted therapeutic agent, chemotherapeutic agent, therapeutic antibody, radiation, cell therapy, gene therapy, and/or immunotherapy.
